(19) United States
(12) Patent Application Publication
(10) Pub. No.: US 2023/0346805 A1
(43) Pub. Date: Nov. 2, 2023

(54) COMPOSITIONS AND METHODS AND USES THEREOF

(71) Applicant: Journey Medical Corporation, Scottsdale, AZ (US)

(72) Inventors: Iain Stuart, Flanders, NJ (US); Flor De Maria Alvarez Mitre, San Luis Potosi (MX); Russell Elliott, Basking Ridge, NJ (US); Gary Lawrence, Raleigh, NC (US); Meital Cohen-Asis, Rishon-Le-Zion (IL); Anat London-Drori, Beer Yakov (IL); Yohan Hazot, Rehovot (IL); Ariel Margulis, Jerusalem (IL)

(21) Appl. No.: 17/597,892

(22) PCT Filed: Jul. 30, 2020
(86) PCT No.: PCT/US2020/044287

§ 371 Date: Jan. 28, 2022

Related U.S. Application Data
(60) Provisional application No. 63/033,085, filed on Jun. 1, 2020.
(60) Provisional application No. 62/881,341, filed on Jul. 31, 2019.

(51) Int. Cl. A61K 31/65; A61K 9/12; A61K 31/192; A61K 47/44; A61K 47/10; A61P 17/10; A61K 9/00; A61K 47/24; A61K 47/46; A61Q 19/00; A61K 8/02; A61K 8/04; A61K 8/92; A61K 8/34; A61K 8/36; A61K 8/42

(52) U.S. Cl. CPC A61K 31/65; A61K 9/122; A61K 31/192; A61K 47/44; A61K 47/10; A61P 17/10; A61K 9/0014; A61K 47/24; A61K 47/46; A61Q 19/00; A61K 8/0245; A61K 8/046; A61K 8/922; A61K 8/92; A61K 8/927; A61K 8/342; A61K 8/361; A61K 8/42; A61K 2800/651; A61K 2800/412

(57) ABSTRACT

The present disclosure relates to compositions, including foamable compositions with improved properties, such as those comprising waxes, emollients, foam adjuvants and/or active agents, and methods for preparing and using them.

This application claims the benefit of priority to U.S. Provisional Patent Application No. 62/881,341, filed Jul. 31, 2019, and 63/033,085, filed Jun. 1, 2020. The contents of both applications are hereby incorporated by reference in their entireties.

The present disclosure relates to compositions and foamable compositions, such as those comprising waxes, emollients, foam adjuvants and/or active agents, and methods for preparing and using them.

Acne is a common category of skin disorders which afflicts many people. The prevalence of adult acne is about 3% in men and between about 11% and 12% in women. Moderate to severe acne is observed in about 14% of acne patients. There are various types of acne recognized in the field, including, for example: acne vulgaris and acne conglobata. Acne vulgaris (cystic acne or simply acne) is generally characterized by areas of skin with seborrhea (scaly red skin), comedones (blackheads and whiteheads), papules (pinheads), pustules (pimples), nodules (large papules) and/or possibly scarring. Acne vulgaris may affect the face, the upper part of the chest, and the back, among other topical areas. Severe acne vulgaris is inflammatory, but acne vulgaris can also manifest in non-inflammatory forms. Acne conglobata is a severe form of acne and may involve many inflamed nodules that are connected under the skin to other nodules. Acne conglobata often affects the neck, chest, arms, and buttocks.

There are typically three levels of acne vulgaris: mild, moderate, and severe. Mild acne vulgaris is characterized by the presence of few to several papules and pustules, but no nodules. Patients with moderate acne typically have several to many papules and pustules, along with a few to several nodules. With severe acne vulgaris, patients typically have numerous or extensive papules and pustules, as well as many nodules.

While mild to moderate acne is often treated topically, using, e.g., retinoids, benzoyl peroxide and some antibiotics, there remains a need for improved topical treatment even for these milder forms of the condition. Treatment for moderate or severe acne typically requires systemic antibiotics like tetracycline and its derivatives (e.g., minocycline and doxycycline) that are given orally or by injection. However, the systemic delivery of tetracycline antibiotics is associated with many adverse side effects, including diarrhea, abdominal cramps, and dizziness. For example, oral tetracycline therapy may induce hyperpigmentation in many organs, including nails, bone, skin, eyes, thyroid, visceral tissue, oral cavity (teeth, mucosa, alveolar bone), sclerae and heart valves. Skin and oral hyperpigmentation have been reported to occur regardless of the amount of time or drug administered, whereas other tissue hyperpigmentation have been reported to occur upon prolonged administration. Skin hyperpigmentation includes diffuse hyperpigmentation as well as over sites of scars or injury. Oral treatments may not be effective against all forms of acne, such as non-inflammatory acne.

Oral tetracyclines are also not indicated for pregnant women or nursing mothers due to teratogenic effects. An example of a commercially available oral tetracycline-based treatment for acne is SOLODYN®. It is indicated to treat only inflammatory lesions of non-nodular moderate to severe acne vulgaris in patients 12 years of age or older. Adverse side effects from the use of SOLODYN® include, inter alia, diarrhea, dizziness, lightheadedness, and nausea, in addition to allergic reactions, bloody stool, blurred vision, rectal or genital irritation, and red, swollen, blistered, or peeling skin. Because of these side effects, the Food and Drug Administration added oral minocycline to its Adverse Event Reporting System (AERS), a list of medications under investigation by the FDA for potential safety issues. Accordingly, there exists a need for topical formulations comprising tetracyclines for the treatment of acne, that do not have the same side effects observed with oral applications.

Developing topical formulations of tetracycline-based antibiotics, e.g., for use in treating acne or other indications, has been challenging for a number of reasons. Firstly, tetracycline-based antibiotics are sensitive to moisture, temperature, and light. Secondly, they are highly susceptible to degradation by a wide range of pharmaceutical carriers and excipients that are typically used as solvents and carriers. Lastly, they are often unstable when suspended in emulsion or dissolved in solution. Thus, formulating useful tetracycline antibiotics, particularly when combined with other active agents such as retinoids, for topical administration requires the identification of a carrier system in which a tetracycline antibiotic remains stable for a sufficiently long period of time for product distribution, storage at a pharmacy, and subsequent therapeutic use by a patient. Such a formulation or carrier system should also allow an active agent, e.g., a tetracycline antibiotic, to penetrate into the skin or mucosa whilst avoiding degradation and preventing the active agent from reaching the blood system in significant or substantial amounts to avoid or minimize potential systemic side effects. In addition, such a formulation or carrier system should also allow an active agent or combination of agents or the carrier itself to remain physically stable without aggregating, clumping, precipitating or separating, and remain homogeneously distributed. Once such a carrier system has been created for tetracycline antibiotics, it may have application for other stable and unstable active agents.

Topical composition comprising for e.g. tetracycline antibiotics may be formulated in any form, e.g., as a liquid, gel, ointment, or a foam. Topical composition comprising for e.g. tetracycline antibiotics may also be foamed. Foams are an increasingly popular delivery system for topical drugs, and may be a platform whereby tetracycline-based antibiotics remain stable for topical administration. Foamable formulations or compositions are typically packaged as a liquid or gel in a pressurized aerosol container together with a propellant, and upon actuation of a valve, the composition is released from the container and forms a foam lattice. During use of an aerosol container, it is sometimes desirable to shake the container to homogenize its contents prior to actuation of the valve and formation of the foam. Shaking an aerosol can may enable the user to gauge the presence of liquid contents within the can. Shaking may also improve the formation of foam by aiding its incorporation into the composition e.g. facilitating emulsifying or dissolving the propellant in the composition. Foamable compositions comprising tetracycline-based antibiotics may therefore benefit from being shakable and flowable such that they can be expelled from a canister or tube without blocking the valve or tube. There remains a need in the art for improved foamable formulations that further enhance some or all of these properties.

### SUMMARY

Described herein are compositions, their applications and uses and methods of making them. The compositions may be carriers and may further comprise one or more therapeutic active agents e.g., tetracycline-based antibiotics (alone or in combination with other active ingredients and/or excipients). The compositions may be formulated in different ways e.g., as a liquid, a foamed liquid, a gel, a foamed gel, an ointment, a foamed ointment, or a foam. The compositions disclosed herein may also be foamable. The compositions described herein may provide for improved stability and/or shakeability and/or usability. Compositions with new crystal structures and ratios are disclosed. Compositions comprising Tmh crystals are provided. Compositions comprising Tmh crystals and plates are provided. Compositions comprising Tmh crystals and plates and spherulites are provided. Compositions comprising plates are provided. Compositions comprising Tmh crystals, for example having higher order crystal structures, may contribute to improved stability and/or also provide for improved shakability. Such compositions are, for example, capable of lowering the melting temperature of the sebum and thereby may also provide for improved ability to liquify or dissolve sebum aiding and allowing for opening of pores and facilitating access of the composition into the pores. Such compositions can also facilitate skin penetration of active agents into the dermis and epidermis. Without being bound to any theory, such compositions when including tetracycline antibiotics may surprisingly help reduce, minimize or not generate antibiotic resistance. Such compositions may facilitate targeting of drug in the sebaceous gland tetracycline, e.g., helping opening pores and directing drug, e.g., a tetracycline antibiotic into the pilosebaceous unit where P. acnes can reside, which in turn may enhance efficacy and safety and moreover may help prevent resistance arising.

In one or more embodiments the technology described herein can have broad application to create formulations with novel Tmh crystals comprising a unique high order wax crystal structure generated through the processes described herein. Crystallization may be undesirable and often a reflection on a poor solvent system. In contrast, for the formulations provided herein, crystallization may provide beneficial properties. Typically, crystallization is more likely to occur in a solvent that is different from the solute to be crystalized. Yet, crystallization in the formulations provided herein is achieved from a solvent (emollient) that is similar or closely similar to the substance to be crystalized. Such similarity can result in crystals with novel crystal fingerprints that provide advantages and improved properties to the formulation. In one or more embodiments, without being bound by any theory, a holding process may contribute to the generation of Tmh crystals and the appearance of a novel crystal fingerprint and in one or more embodiments so does its application to formulating wax in emollient and the process conditions.

In one or more embodiments, crystallization in the formulations provided herein does not result in a new polymorphic structure but, rather, retains the same polymorphic form, e.g. beta (β) remains beta (β) or beta′ (β′) remains beta′ (β′). In one or more embodiments crystallization by a holding process may result in a minor polymorphic structure change. In one or more embodiments, crystallization in the formulations provided herein results in a new microstructure and/or a new crystal fingerprint. In some embodiments the Tmh crystal structure formed by a holding process may on average be less dense e.g., with a less concentrated packing arrangement, than the crystal structure comprising spherulites formed by a continuous heating-cooling process. In some embodiments the Tmh crystal structure formed by a holding process may on average occupy a higher percentage of the area measured than the crystal structure comprising spherulites formed by a continuous heating-cooling process. In some embodiments the Tmh crystal structure of the formulations disclosed herein may have a similar or the same density to that of the spherulites. In some embodiments the Tmh crystals on average occupy more space and/or are larger than those of the spherulites. In some embodiments the Tmh crystal structure has stronger and or more interactions (inter-crystal and/or intra-crystal), resulting in a stronger and/or more stable crystal structure. In some embodiments there are more Van Der Vaals interactions in the crystal structure. In some embodiments there are more hydrogen bonds/interactions in the crystal structure. In some embodiments there are more hydrophobic interactions leading to a more stable crystal structure which allows for improved fluidity (or flowability).

In some embodiments the Tmh crystal structure in a formulation disclosed herein displays an increase in enthalpy. In some embodiments the Tmh crystal structure results in a significant upward shift in the highest melting point (as measured by DSC) in the carrier and/or in a pharmaceutical composition disclosed herein (e.g., one with minocycline hydrochloride (MCH) and adapalene (ADP) as active pharmaceutical agents in addition to carrier excipients). In some embodiments the formulation is a carrier without an active agent. In some embodiments the formulation is a composition with one or more therapeutic agents. In some embodiments the therapeutic agent is an active pharmaceutical agent, a cosmeceutical, a cosmetic agent, and/or combinations of one or more of active pharmaceutical agents, cosmeceutical and cosmetic agents. In one or more embodiments the compositions can, when applied topically to the skin, facilitate the breakdown and or dissolution of sebum, e.g., by lowering the melting temperature of the sebum. In one or more embodiments the compositions can, when applied topically to the skin, facilitate improving penetration and delivery into rather than through the skin or mucosa or body cavity wall.

In one or more embodiments, Tmh crystals disclosed herein may be characterized and/or identified by a particular phase transition TM4 temperature, alone or in combination with other properties such as an SRS (i.e., raman) spectra peak with shoulders, and/or a particular FITR wavelength.

In one or more embodiments there is provided composition comprising a tetracycline antibiotic and/or a retinoid, and a wax e.g. hydrogenated castor oil, wherein the wax, such as hydrogenated castor oil, is present in the composition in an amount and form effective to produce a formulation with crystals of a higher melting temperature as compared to those present in formulations prepared by a continuous heating-cooling process. In one or more embodiments, the formulation is characterized by an improved fluidity and/or shakability that is maintained for 6 months or more at 25° C.. In some embodiments, the formulation comprises a wax other than hydrogenated castor oil. In one or more embodiments, the formulation is characterized by an improved fluidity and/or shakability that is maintained for about 3 months, or about, 4 months, or about 5 months, or about 6 months, or about 7 months, or about 8 months, or about 9 months about 10 months, or about, 11 months, or about 12 months, or about 15 months, or about 18 months, or about 21 months, or about 24 months or more at 25° C.. In one or more embodiments, the formulation is characterized by an improved fluidity and/or shakability that is maintained for any one or more of the aforesaid time periods at 5° C.. In one or more embodiments, the formulation is characterized by an improved fluidity and/or shakability that is maintained for 3 months or more at 40° C..

### BRIEF DESCRIPTION OF THE FIGURES

The disclosure will be better understood from a reading of the following detailed description taken in conjunction with the drawings below:

FIG. 1. Differential scanning calorimetry (DSC) thermograms for model systems created through stepwise addition of the different formulation components.

FIG. 2A. Different structures in formulations prepared by a continuous heating-cooling process and a holding process.

FIG. 2B. Photomicrographs for placebo formulations with 1.2% hydrogenated castor oil (HCO) prepared by a continuous heating-cooling process or a holding process and kept at 25° C. on day 0.

FIG. 2C. Crystal count, sizes and amounts (%) of different crystal structures in formulations prepared by a continuous heating-cooling process and a holding process.

FIG. 2D. Photomicrographs for placebo formulations with 1.2% HCO prepared by a continuous heating-cooling process or a holding process and heated from 25° C. to 80° C. on day 0.

FIG. 2E. Photomicrographs for placebo formulations with 2% HCO prepared by a continuous heating-cooling process or a holding process and kept at 25° C. on day 0.

FIG. 2F. Photomicrographs for formulations with 1.2% HCO comprising MCH+ADP prepared by a holding process followed by the addition of heptane instead of a propellant and kept at 25° C. on day 0.

FIG. 2G. Photomicrographs for collapsed foams from formulations with 1.2% HCO comprising MCH+ADP prepared by a continuous heating-cooling process or a holding process.

FIG. 2H. DSC thermograms for formulations with 1.2% HCO comprising MCH+ADP prepared by a continuous heating-cooling process or a holding process and kept at 5° C. for 15 and 30 days.

FIG. 2I. DSC thermograms for formulations with 1.2% HCO comprising MCH+ADP prepared by a continuous heating-cooling process or a holding process and kept at 25° C. for 0, 15 and 30 days.

FIG. 2J. DSC thermograms for formulations with 1.2% HCO comprising MCH+ADP prepared by a continuous heating-cooling process or a holding process and kept at 40° C. for 0, 15 and 30 days.

FIG. 2K. DSC thermograms for placebo formulations with 1.2% HCO prepared by a continuous heating-cooling process or a holding process and kept at 5° C., 25° C. or 40° C. for 0 and 15 days.

FIG. 2L. Formulations comprising MCH+ADP with 1.2% HCO prepared by a continuous heating-cooling process (yellow and grey lines representing two replicates) and a holding process (blue and orange lines representing two replicates), kept at 40° C. and analyzed on day 0 by small angle X-ray scattering.

FIG. 2M. Formulations comprising MCH+ADP with 1.2% HCO prepared by a continuous heating-cooling process (Blue line) and a holding process (Orange line), kept at 25° C. and analyzed on Day 0 by wide angle X-ray scattering.

FIG. 2N. FIG. 2N. Flow point for foam formulations comprising MCH+ADP with 1.2% HCO manufactured by a continuous heating-cooling process and a holding process, and kept at 40° C. for 0, 15 and 30 days.

FIG. 3A. DSC for Sebum Experiments.

FIG. 3B. Photomicrographs of sebum side by side with MAH, a placebo formulation (PCO) and a formulation comprising MCH prepared by a continuous heating-cooling process (MCO) and Oil-in-water emulsion (OIWE) at 25° C., heated to 35° C., and held at to 35° C. for 1 minute.

FIG. 4A. Formulation with 1.2% or 2% HCO prepared by a continuous heating-cooling process and a holding process and kept at 5° C., 25° C. and 40° C. for 15 days.

FIG. 4B. Formulation with 1.2% or 2% HCO prepared by a continuous heating-cooling process and 1.2% HCO prepared by a holding process and kept at 5° C., 25° C. and 40° C. for 30 days.

FIG. 5. DSC thermograms for formulations with 1.2% or 2% HCO comprising MCH+ADP, kept at 40° C. for 15 days and prepared by three different processes: (1) a holding process, (b) holding at 52° C. and heating to 65° C., or (c) adding HCO at 22° C.

FIG. 6A. Formulations prepared at different holding temperatures and kept at 25° C. (upper panel) or 40° C. (lower panel) on Day 0.

FIG. 6B. T_{M4} for formulations prepared at different holding temperatures and kept at 25° C. or 40° C. on Day 0.

FIG. 6C. T_{M4} for placebo formulations prepared at different holding temperatures, and a formulation prepared using a continuous heating-cooling process, and kept at 25° C. on Day 0.

FIG. 6D. Size (μm²) of Tmh crystals and spherulites prepared at different holding temperatures.

FIG. 6E. Relative percentages (by area) of Tmh crystals and spherulites prepared at different holding temperatures.

FIG. 6F. Crystal counts of Tmh crystals and spherulites prepared at different holding temperatures.

FIGS. 6G to 6L. Photomicrographs of crystals taken at 50° C. in samples manufactured at different holding temperatures.

FIG. 7A. Flow point temperatures for samples stored at 25° C. with different active agent(s) (API) and HCO levels through storage time.

FIG. 7B. Flow point temperatures for samples stored at 40° C. with different active agent(s) (API) and HCO levels through storage time.

FIGS. 8A to 8C. G′ values for samples stored at 25° C. containing MCH+ADP with 2% HCO vs. 1.2% HCO through storage time.

FIG. 9. G′ values for samples stored at 40° C. with different active agent(s) (API) through storage time.

FIG. 10. Viscosity values for samples stored at 25° C. containing MCH and ADP with 2% HCO and with 1.2% HCO through different storage times.

FIG. 11. Enthalpy change of samples with 2% HCO and 1.2% HCO with and without API prepared by holding process and a formulation prepared by a continuous heating-cooling process.

FIG. 12A. Photomicrograph of Tmh crystals imaged by 50× objective.

FIG. 12B. Photomicrograph of Tmh crystals imaged by 20× objective.

FIG. 12C. Photomicrograph of Tmh crystals imaged by 50× objective at 45° C., following mixture of a sample formulation with acetone.

FIG. 12D. Photomicrograph of Tmh crystals imaged by 50× objective under slow heating (1° C./min), following mixture of a sample formulation with hexane.

FIG. 13A. Images of glass vials of formulations with different wax ratios stored at 25° C. and 40° C.

FIG. 13B. DSC thermograms for upper and lower fractions of a formulation with 1.2% HCO and 0.1% beeswax stored at 40° C. for 30 days.

FIG. 13C. DSC thermograms for a formulation with 2% HCO and 0.1% beeswax.

FIG. 13D. DSC thermograms for a formulation with 0.6% HCO and 2% beeswax.

FIG. 13E. DSC thermograms for a formulation with 2% HCO and 2% beeswax.

FIG. 13F. DSC thermograms for a formulation with 2% HCO and 1.2% beeswax.

FIG. 13G. DSC thermograms for a formulation with 1.2% HCO and 2% beeswax.

FIG. 13H. Images of glass vials of formulations with alternative waxes stored at 25° C. and 40° C.

FIG. 13I. DSC thermograms for upper and lower fractions of a formulation with emulsifying wax stored at 40° C. for 30 days.

FIG. 13J. Photomicrograph of upper and lower fractions of a formulation with emulsifying wax.

FIG. 14A. DSC thermograms for a formulation with 23.6% soybean oil and 50% coconut oil.

FIG. 14B. DSC thermograms for a formulation with 50% soybean oil and 23.6% isopropyl myristate oil.

FIG. 14C. DSC thermograms for a formulation with 70% soybean oil and 23.6% coconut oil.

FIG. 14D. DSC thermograms for a formulation with 50% soybean oil and 23.6% MCT oil.

FIG. 15A. DSC thermograms for mixtures of different oils and HCO.

FIG. 15B. Photomicrographs of a mixture of HCO and coconut oil.

FIG. 15C. Photomicrographs of a mixture of HCO and octyldodecanol.

FIG. 16A. Images of glass vials of formulations with 1.2% HCO and 2% beeswax prepared by a holding step of 4 hours or about 16 hours (overnight holding) at 25° C. and at 40° C.

FIG. 16B. DSC thermograms for upper and lower fractions of a formulation with 1.2% HCO and 2% beeswax prepared by an overnight holding step stored at 40° C. for 15 days.

FIG. 16C. Photomicrograph of upper and lower fractions of a formulation 1.2% HCO and 2% beeswax prepared by an overnight holding step stored at 40° C. for 15 days and taken at 50° C.

FIG. 17A. Full FTIR spectra for formulation oils (Coconut oil+soybean oil+mineral oil+cyclomethicone), neat HCO, oils+1.2% HCO, and formulations with 1.2% HCO prepared in a holding process.

FIG. 17B. FTIR spectra for formulation's oils (Coconut oil+soybean oil+mineral oil+cyclomethicone), neat HCO and oils+1.2% HCO.

FIG. 17C. FTIR spectra measured through increasing temperatures for formulations with 1.2% HCO prepared in a holding process vs. a continuous heating-cooling process.

FIG. 17D. FTIR spectra measured at 25° C. and 50° C. for formulations with 1.2% HCO prepared in a holding process vs. a continuous heating-cooling process.

FIG. 17E. FTIR spectra measured at 25° C. and 50° C. for formulations with 2% HCO prepared in a holding process vs. a continuous heating-cooling process.

FIG. 17F. Frequency of band 1 for formulations with 1.2% or 2% HCO prepared by a holding process or a continuous heating-cooling process.

FIG. 18. 20× magnification image of minocycline and adapalene with 1.2% HCO and 2% beeswax prepared by a holding process while heating at 5° C./min.

FIG. 19. Average release rate results for minocycline and adapalene.

FIG. 20A. SRS (raman) spectra of vehicle batch.

FIG. 20B. SRS spectra of vehicle batch.

FIG. 21. DSC and shear for batches prepared by different processes.

FIG. 22A. Images observed at 25° C. for batch manufactured with holding and shear.

FIG. 22B. 50×image of batch manufactured with shear.

FIG. 22C. Few Tmh Crystals observed under 50× objective at 45-50° C. when manufacture was with shear.

### DETAILED DESCRIPTION

Non-limiting, exemplary embodiments are discussed herein.

Provided herein in one or more embodiments are compositions (carriers and/or therapeutic compositions), methods of making them, their applications and uses, e.g., for treating acne. In some embodiments the compositions are foamable. In one or more embodiments the therapeutic compositions may comprise one or more active agents e.g., comprising tetracycline-based antibiotics. In one or more embodiments the compositions may provide superior stability and/or fluidity and/or shakability and/or ability to dissolve sebum. In some embodiments the compositions provide superior penetration into the skin. In some embodiments the compositions provide improved targeting and or delivery into the pilosebaceous unit. When formulated as a foamable composition and packaged in an aerosol container, characteristics like superior fluidity and/or shakability are additionally advantageous.

### Definitions

Unless defined otherwise, all technical and scientific terms used herein have the same meaning as is commonly understood by one of ordinary skill in the art to which this disclosure belongs. All ranges disclosed herein include the endpoints. The use of the term “or” shall be construed to mean “and/or” unless the specific context indicates otherwise. All patents, applications, published applications, and other publications are incorporated by reference in their entirety. In the event that there is a plurality of definitions for a term herein, those in this section prevail unless stated otherwise.

All % values are provided on a weight per weight (w/w) basis.

Various carriers and compositions or formulations are described herein. They are often described for use in a method. A reference to or example of a carrier, composition or formulation for use in one method does not in any way limit the carrier, composition or formulation for use just in that method, but it can be for use in any other method or embodiment described herein. The carriers, compositions or formulations described herein are in one or more embodiments provided as carriers, compositions or formulations and are in one or more embodiments provided as a product even where they are described only in relation to their use in a method.

As used herein, the term “about” has its usual meaning in the context of pharmaceutical and cosmetic formulations to allow for reasonable variations in amounts that can achieve the same effect, typically plus or minus up to 30%. For example, if an amount of “about 1” is provided, then the amount can be up to 1.3 or from 0.7. In cases where “about X” will lead to a figure of above 100%, the term in one or more embodiments can be read as reflecting up to 100% by weight less the total of the minimum amount of the other ingredients. Likewise, it will be appreciated by one skilled in the art to the extent X is reduced from that upper level the amounts of the other ingredients are increased appropriately. As will be appreciated by one of skill in the art, there is some reasonable flexibility in formulating compositions such that where one or more ingredients are varied, successful formulations can still be made even if an amount falls slightly outside the range. Therefore, to allow for this possibility, amounts are qualified by about. In one or more other embodiments, the figures can be read without the term “about.”

As used herein, the terms “composition(s)” and “formulation(s)” can be used interchangeably depending on the context in which they are used as would be appreciated by a person skilled in the art.

The term “room temperature” as used herein, means 20° C. to 25° C. In an embodiment it is 20° C. In an embodiment it is 21° C. In an embodiment it is 22° C. In an embodiment it is 23° C. In an embodiment it is 24° C. In an embodiment it is 25° C. The term “ambient conditions” as used herein means room temperature, pressure and humidity. Ambient temperature and room temperature are used interchangeably herein.

The term “thixotropic,” as used herein, means that the formulation shows a decrease in viscosity upon application of a shear force. The structure of the formulation breaks down, leading to a reduction in viscosity. When the formulation is left standing without shear force, the viscosity is recovered over time.

As used herein, the term “gel” means a jelly-like material. Gels can be in a liquid, a semi-liquid, or a semi-solid state. The gel can be a liquid gel where the amount of gelling agent or gelling effect is lower, such that the gel structure or connections are weaker or looser so that when placed in a tube and tilted from a vertical position to a horizontal position, the gel more readily flows and adapts to the horizontal position. The rheological properties of gels at different surface temperatures can influence the release and bioabsorption of drugs therefrom.

The term “liquid gel”, refers, inter alia, to a formulation where the gel is loose or fluid or such that when subjected to gravity, it will pour or become liquid.

As used herein, “foam” has its ordinary meaning to one of skill in the art, e.g., it may refer to an object or substance formed by trapping gas pockets within a solid or liquid. The gas pockets may comprise a gas, e.g., oxygen, nitrogen, or a mixture of gases, e.g., helium and xeon, or atmospheric air or alkanes gases. The gas pockets within the foam may be connected to each other, e.g., closed-cell foams or discrete, e.g., open-cell foams. As used herein, “foamable compositions” refers to any composition that has the ability to form a foam. In some embodiments, foamable compositions comprise a carrier with or without a liquefied or compressed gas propellant, that forms a foam when the carrier is brought in contact with the propellant or by mechanical means, such as an air pump. In some embodiments, a foamable composition is packaged in an aerosol container together with a pressurized propellant. In some embodiments the foamable composition is separate from the propellant such as in a bag in can system. In some embodiments, a valve on the aerosol container is actuated to release the foamable composition to form a foam.

In some embodiments, a formulation disclosed herein comprises water. In some embodiments, a formulation disclosed herein is water free. As used herein, the terms “waterless” or “water-free,” refer to compositions that contains no free or unassociated or absorbed water. In some embodiments, a waterless or water-free composition comprises 0.0% added water by weight. Such a composition may contain trapped, bound, associated or otherwise unfree water, e.g., within its higher order crystal structure. The terms “essentially waterless” or “essentially water-free” refer to compositions that comprise less than 0.05% of water by weight. In some embodiments, an essentially water-free composition comprises 0.04%, 0.03%, 0.02%, or 0.01% water by weight. The terms “substantially water-free” or “substantially waterless” compositions that comprise less than 0.5% of water by weight. In some embodiments, a substantially water-free composition comprises 0.4%, 0.3%, 0.2%, or 0.1% water by weight. As used herein, “low water” refers to a composition that contains about or less than 1% of water by weight. In some embodiments, a composition with low water comprises 0.9%, 0.8%, 0.7%, 0.6% or 0.5% of water by weight.

The term “single phase” as used herein means that, after preparation the liquid components of the composition or carrier are fully miscible, and the solid components, if any, are either dissolved or homogeneously suspended in the composition so that only one phase is visible. In the context of a foamable composition “single phase” means that, after addition of propellant to the composition or carrier, the liquid components of the foamable composition or carrier are fully miscible, and the solid components, if any, are either dissolved or homogeneously suspended in the composition so that only one phase is visible. In some embodiments a composition has a single phase before addition of propellant. In some embodiments, a composition has a single phase after addition of propellant.

By the term “substantially a single phase” it is meant that the composition or carrier, after preparation, is primarily or essentially a single phase as explained above, but can also have present a small amount of material which is capable of forming a separate phase amounting to less than about 5% by weight of the composition or carrier after the addition of propellant, e.g., less than about 3% by weight, or less than about 1% by weight of the composition. In the context of a foamable composition by the term “substantially a single phase” it is meant that the composition or carrier, after addition of propellant, is primarily or essentially a single phase as explained above, but can also have present a small amount of material which is capable of forming a separate phase amounting to less than about 5% by weight of the composition or carrier after the addition of propellant, e.g., less than about 3% by weight, or less than about 1% by weight of the composition. In some embodiments a composition may be substantially a single phase before addition of propellant and a substantially single phase after addition of propellant. In some embodiments a composition may be substantially a single phase before addition of propellant and a single phase after addition of propellant. In some embodiments a composition may be a single phase before addition of propellant and substantially a single phase after addition propellant.

The term “unstable” or “chemically unstable” as used herein, refers to a compound, e.g., an agent, which is oxidized, degraded, and/or reacts within a day, upon exposure to air, light, skin, water, any pharmaceutical excipient, or any active agent under ambient conditions. In some embodiments, an unstable compound is partially (e.g., 5%, 10%, 50%, 75%) or fully degraded in less than 24 hours, e.g., less than 16 hours, less than 12 hours, less than 6 hours, less than 5 hours, less than 4 hours, less than 3 hours, less than 2 hours, or less than 1 hour, upon exposure to air, light, skin, water, or any pharmaceutical excipients under ambient conditions or any active agent. The term “physically unstable” as used herein refers to a substance or compound e.g., an agent which within a day aggregates, clumps, sediments, separates out or otherwise changes its physical state under ambient conditions within a day. It can also refer to a carrier which within a day changes its physical state under ambient conditions. In some embodiments, a physically unstable compound or composition changes its physical state in less than 24 hours, e.g., less than 16 hours, less than 12 hours, less than 6 hours, less than 5 hours, less than 4 hours, less than 3 hours, less than 2 hours, or less than 1 hour.

The term “unstable active agent” or “chemically unstable active agent” as used herein, refers to an active agent (e.g., minocycline HCl, or adapalene), or a part thereof which is oxidized, degraded, and/or reacts (“undergoes change”) within a day upon exposure to air, light, skin, water, or a pharmaceutical excipient under ambient conditions. In some embodiments, an unstable active agent or a part thereof undergoes change in less than 24 hours, e.g., less than 16 hours, less than 12 hours, less than 6 hours, less than 5 hours, less than 4 hours, less than 3 hours, less than 2 hours, or less than 1 hour, upon exposure to air, light, skin, water, or any pharmaceutical excipients under ambient conditions. The term “physically unstable active agent” as used herein refers to a substance or compound e.g., an active agent, or a part thereof which aggregates, clumps, sediments, separates out or otherwise changes its physical state under ambient conditions within a day. It can also refer to a composition comprising one or more active agents which within a day changes its physical state under ambient conditions. In some embodiments, a physically unstable active agent changes its physical state in less than 24 hours, e.g., less than 16 hours, less than 12 hours, less than 6 hours, less than 5 hours, less than 4 hours, less than 3 hours, less than 2 hours, or less than 1.

It should be noted that the terms “surfactant,” “surface active agent,” and “emulsifier” in the context used herein, refer to compounds that on their own, can both reduce surface tension between two substances or phases, and also stabilize an emulsion of water and oil. Reduction of surface tension in foam technology changes a material's ability to create small stable bubbles. Surfactants include non-ionic, ionic, anionic, cationic, zwitterionic, amphoteric and amphiphilic surfactants. Surfactants may be derivatives of fatty alcohols or fatty acids, such as ethers or esters formed from such fatty alcohols or fatty acids with hydrophilic moieties, such as polyethylene glycol (PEG).

“Surfactant,” “emulsifier,” and “surface active agent,” as used herein, do not include compounds which do not function effectively on their own to reduce surface tension between two substances or phases and stabilize an emulsion of water and oil. For instance, a native (non-derivatized) fatty alcohol or fatty acid, as well as a wax, generally does not reduce surface tension between two substances or phases or stabilize an emulsion of water and oil on its own, and therefore is not considered a surfactant in the context used herein. Likewise, for example, propoxylated lanolin oil derivatives are not themselves surfactants or emulsifiers. These excipients may be used in combination with or in lieu of a surfactant in some embodiments of the formulations disclosed herein. In some embodiments, foam adjuvants in formulations disclosed herein comprise fatty acids and/or fatty alcohols. In some embodiments, formulations disclosed herein comprise emollients comprising propoxylated lanolin oil derivatives.

As used herein, the term “emollient” refers to a material or agent that, when placed in contact with the human skin, is able to soften, smoothen, reduce scaling and itching, reduce inflammation, improve skin barrier function, and/or act as a carrier for active agents. An emollient can comprise one or more oils. Examples of emollients include but are not limited to avocado oil, isopropyl myristate, mineral oil, capric triglycerides, capryllic triglyceride, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohols, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, olive oil, corn oil, soybean oil, canola oil, cottonseed oil, coconut oil, sesame oil, safflower oil, sunflower oil, borage seed oil, syzigium aromaticum oil, hempseed oil, herring oil, cod-liver oil, salmon oil, flaxseed oil, wheat germ oil, evening primrose oil, an essential oil, a silicone oil, dimethicone, cyclomethicone, polyalkyl siloxane, polyaryl siloxane, polyalkylaryl siloxane, a polyether siloxane copolymer, and poly(dimethylsiloxane)-(diphenyl-siloxane).

The term “co-surfactant” as used herein refers to a molecule which on its own is not able to form and stabilize an oil-in-water emulsion, but when used in combination with a surfactant as defined herein, the co-surfactant can help a surfactant create an emulsion and can boost the stabilizing power or effect of the surfactant. Examples of co-surfactants include fatty alcohols, such as cetyl alcohol, or fatty acids, such as stearic acid. Cetyl alcohol is a waxy hydrophobic substance that can be emulsified with water in combination with a surfactant. Some substances can have more than one function and for example, fatty alcohols can in some formulations act as a co-solvent. In some embodiments, a co-surfactant can itself be converted into a surfactant or soap by, for example, adding a base, such as, triethanolamine to a fatty acid like stearic acid.

The term “viscosity-modifying agent” in the context of the present disclosure is an agent which, when added to a hydrophobic oil, facilitates the creation of a hydrophobic breakable vehicle in the form of a breakable gel or breakable foam. As used herein, the viscosity-modifying agent, in relation to a foamable composition, is also referred to as a “foamer complex,” a “foam stabilizer” or a “foam adjuvant”, comprising, e.g., a fatty alcohol, a fatty acid and/or a wax. In some embodiments the foam adjuvant is a fatty alcohol and a wax or a fatty acid and a wax. In some embodiments it is a wax. In some embodiments, the foam adjuvant or viscosity modifying agent comprises at least one of a fatty alcohol, a wax or a fatty acid. In some embodiments, the foam adjuvant or viscosity modifying agent is selected from a group consisting of a fatty alcohol, a wax and a fatty acid. In some embodiments, the foam adjuvant is a fatty alcohol. In some embodiments, the foam adjuvant is a fatty acid. In some embodiments, the foam adjuvant is a wax. In some embodiments, a wax has the properties of a foam adjuvant. In some embodiments a fatty alcohol, and/or a fatty acid and/or a wax is an adjuvant. In the context of the present disclosure fatty alcohols, fatty acids and waxes that are compatible with tetracycline antibiotics, and in particular with a minocycline or a doxycycline, are compatible adjuvants. In some embodiments, compatible adjuvants comprise fatty alcohols, fatty acids and waxes compatible with retinoids, and in particular with adapalene.

As used herein, a formulation disclosed herein may include one or a combination of waxes. In some embodiments, a wax may have a melting point temperature of about 36° C. or higher. In some embodiments, a wax may have a melting point temperature of about 40° C. or higher. In some embodiments, a wax may have a melting point temperature of about 49° C. or higher. In some embodiments, a wax may have a melting point temperature of about 81° C. or higher. In some embodiments, a wax may have a melting point temperature of about 83° C. or higher. In some embodiments, a wax may have a melting point temperature of about 88° C. or higher. In some embodiments, a wax may have a melting point temperature of about 61° C. or higher. In some embodiments, a wax may have a melting point temperature of about 65° C. or higher. In some embodiments, a wax may have a melting point temperature of about 50° C. or higher. In some embodiments, a wax may have a melting point temperature of about 54° C. or higher. In some embodiments, a wax may have a melting point temperature of about 57° C. or higher. In some embodiments, a wax may have a melting point temperature of about 60° C. or higher. In one or more embodiments, the formulations provided herein comprise a wax, wherein the wax within the formulation has a melting point of 68-69° C. In one or more embodiments, the formulations provided herein comprise a wax, wherein the wax within the formulation has a melting point of 42-44° C. In some embodiments, a wax may have a melting point temperature of about 83-88° C. In some embodiments, a wax may have a melting point temperature of about 61-65° C. In some embodiments, a wax may have a melting point temperature of about 50-54° C. In some embodiments, a wax may have a melting point temperature of about 57-60° C.

The term “breakable” refers to a property of a gel or foam wherein the gel or foam is stable upon dispensing from a container yet breaks and spreads easily upon application of shear or mechanical force, which can be mild, such as a simple mechanical rub.

The term “water activity” as used herein represents the hygroscopic nature of a substance, or the tendency of a substance to absorb water from its surroundings. Microorganisms require water to grow and reproduce, and such water requirements are best defined in terms of water activity of the substrate. The water activity of a solution is expressed as Aw=P/Po, where P is the water vapor pressure of the solution and Po is the vapor pressure of pure water at the same temperature. Every microorganism has a limiting Aw, below which it will not grow; e.g., for Streptococci, Klebsiella spp, Escherichia coli, Clostridium perfringens, and Pseudomonas spp, the Aw value is 0.95. Staphylococcus aureus is most resistant and can proliferate with an Aw as low as 0.86, and fungi can survive at an Aw of at least 0.7. The identification of a “solvent,” as used herein, is not intended to characterize the solubilization capabilities of the solvent for any specific active agent or any other component of the composition or foamable composition. Rather, such information is provided to aid in the identification of materials suitable for use as a component of the composition or foamable composition described herein.

The terms “hydrophobic gel composition” or “hydrophobic foamable composition” or “hydrophobic foam composition” or “hydrophobic composition” as used herein refer to compositions that have a low solubility in water. In some embodiments, 100 to 1000 parts of water are needed to dissolve or render miscible 1 part of the composition. In some embodiments, 1000 to 10,000 parts of water are needed to dissolve or render miscible 1 part of the composition. In some embodiments, more than 10,000 parts of water are needed to dissolve or render miscible 1 part of the composition.

It should be noted that the term “substantially free of” an ingredient as used herein, is intended to mean that the composition comprises less than about 0.5% by weight of the ingredient unless specifically indicated otherwise.

As used herein, the term “essentially free of” an ingredient as used herein, is intended to mean that the composition comprises less than about 0.05% by weight of the ingredient, unless specifically indicated otherwise.

As used herein, the term “free of” an ingredient used herein, is intended to mean that the composition does not comprise any amount of the ingredient, unless specifically indicated otherwise e.g. where the ingredient is present in a trapped, bound, associated or otherwise unfree state.

The terms “surfactant-free” or “emulsifier-free” or “non-surfactant” refer to compositions which comprise no or negligible levels of surfactants, emulsifiers, or surface-active agents. Where a formulation includes insignificant or de minimis amounts of surfactants, emulsifiers, or surface-active agents it is considered to be essentially surfactant-free. As used herein, “essentially free” indicates less than about 0.05% by weight of a surfactant, e.g., a surfactant selected from the group consisting of non-ionic, ionic, anionic, cationic, zwitterionic, amphoteric and ampholytic surfactants. The term “substantially surfactant-free” relates to a composition that contains a total of about or less than 0.5% by weight a surfactant, e.g., a surfactant selected from the group consisting of non-ionic, ionic, anionic, cationic, zwitterionic, amphoteric and ampholytic surfactants. In some embodiments, the composition comprises about or less than 0.2% by weight of a surfactant; about or less than 0.15% by weight; about or less than 0.1% by weight; about or less than 0.05% by weight; or about or less than 0.01% by weight.

As used herein, the term “preventing” refers to avoiding the onset of a disorder or condition from occurring in a subject that has not yet been diagnosed as having the disorder or condition, but who may be susceptible to it.

As used herein, the term “treatment” or “treating” refers to inhibiting, reversing, ameliorating, or reducing the disorder or condition, e.g., arresting its development; relieving the disorder or condition, e.g., causing regression of the disorder or condition or reversing the progression of the disorder or condition; slowing progression, or relieving or reducing one or more symptoms of the disorder or condition. In some embodiments it can also mean preventing or helping to prevent the disorder or condition or one or more symptoms thereof.

It should be noted that the term “a method of preventing or treating a disease or a disorder” as provided throughout the specification is interchangeable with the term “use of the composition as a medicament for preventing or treating a disease.” It should be noted that the term “disease” is used interchangeably with the term “disorder.”

By “de minimis” it is meant to be so minor that its effect is to be disregarded, e.g., having no functional impact on a formulation or method.

The term “clinical response to treatment”, (“clinical success” or “clinical failure”) in the context of acne conglobate, acne vulgaris or rosacea treatments is derived from efficacy evaluation endpoints. The term “lesion count” relates to the number of inflammatory lesions (e.g., papules and pustules) and/or the number of non-inflammatory lesions (e.g., open and closed comedones, also known as blackheads and whiteheads) present in a designated area of the body (e.g., in case of face, on the forehead, left and right cheeks, nose and chin).

In some embodiments, primary efficacy endpoints are: (1) the proportion of patients achieving treatment success at Week 12 based on an Investigator's Global Assessment (success is defined as a score of “clear” or “minimal” (e.g., IGA score of 0 or 1 respectively), and at least a two-category improvement from baseline), (2) the mean change from baseline in inflammatory lesion counts in each treatment group at Week 12, and (3) the mean change from baseline in non-inflammatory lesion counts in each treatment group at Week 12. Safety evaluations may include reported adverse events, local skin tolerability assessments, physical examinations, and vital signs.

A clinical response or efficacy as used herein may refer, in some embodiments, to a quantifiable improvement in the severity of a disease, e.g., acne conglobate, acne vulgaris or rosacea, after the start of treatment, e.g., 4 weeks, 8 weeks, or 12 weeks after, compared to the baseline before treatment, or wherein according to any of the aforementioned endpoints a statistically significant reduction or improvement is demonstrated as compared to placebo. In some embodiments, treatment efficacy is assessed by the absolute change in inflammatory lesion count after the start of treatment, e.g., 4 weeks, 8 weeks, or 12 weeks after, compared to the baseline before treatment. In some embodiments, treatment efficacy is assessed by a 2-grade decrease on the Investigator Global Assessment (IGA) scale after the start of treatment and a score of 0 or 1 on the IGA scale after the start of treatment, e.g., 4 weeks, 8 weeks, or 12 weeks after, compared to the baseline before treatment. In some embodiments, treatment efficacy is assessed by the absolute change in non-inflammatory lesion count after the start of treatment, e.g., 4 weeks, 8 weeks, or 12 weeks after, compared to the baseline before treatment. In some embodiments, treatment efficacy is assessed by a 2-grade decrease on the Investigator Global Assessment (IGA) scale after the start of treatment, e.g., 4 weeks, 8 weeks, or 12 weeks after, compared to the baseline before treatment. In some embodiments, treatment efficacy is assessed by a score of 0 or 1 on the IGA scale after the start of treatment, e.g., 4 weeks, 8 weeks, or 12 weeks after, compared to the baseline before treatment. In some embodiments, treatment efficacy is assessed by the absolute change in inflammatory lesion count with treatment compared to placebo. In some embodiments, treatment efficacy is assessed by the absolute change in non-inflammatory lesion count with treatment compared to placebo. In some embodiments, treatment efficacy is assessed by a 2-grade decrease on the IGA scale with treatment compared to placebo. In some embodiments, treatment efficacy is assessed by a score of 0 or 1 on the IGA scale with treatment compared to placebo. In some embodiments, treatment efficacy is assessed by a 2-grade decrease on the IGA scale with treatment compared to placebo and a score of 0 or 1 on the IGA scale with treatment compared to placebo.

In one or more embodiments, administration of a combination product disclosed herein (e.g., minocycline and adapalene) is superior to vehicle (i.e. placebo) administration for one or more of the following endpoints: (1) proportion of subjects (%) with Investigator's Global Assessment (IGA) treatment success (IGA score of 0 or 1) and/or a 2-grade decrease on the IGA scale with treatment; and (2) absolute change from baseline in mean inflammatory and non-inflammatory lesion counts at Week 12. In one or more embodiments treatment with a combination product achieves a statistically superior result. For example, greater than 25-45% of patients receiving combination treatment may achieve IGA treatment success, e.g., about 35.9%, as compared to 10-20%, e.g., about 15.7%, of patients in the vehicle treatment group. In some embodiments, numerical superiority may be observed for efficacy endpoints for these comparisons at Week 12.

In one or more embodiments the combination product is superior numerically to vehicle for the endpoint of absolute reduction in non-inflammatory lesion counts at Week 12. For example, a mean lesion count reduction of about 25-30, e.g., about −25.9 (−53.83%) for the combination treatment group may be superior when compared to a reduction of about 15-25, e.g., about −24.1 (−48.09%) for the vehicle treatment group.

In one or more embodiments the absolute reduction in non-inflammatory lesion counts at Week 12 for the combination product is numerically superior compared to the product with 3% minocycline only or to the product with 0.3% adapalene only. For example, the combination product may be superior to each of (1) 3% minocycline alone foam and (2) 0.3% adapalene alone foam.

In some embodiments, a method of treating acne is provided, comprising topically administering a foamable composition at least once daily for at least 12 weeks to a target area on the patient having acne, wherein said administration results in a reduction of lesion count from baseline or a reduction in the percent change of number of lesions from baseline, and wherein the foamable composition comprises a hydrogenated castor oil, one or more foam adjuvants and or one or more waxes, e.g., lacking an active agent such as minocycline and/or adapalene. In some embodiments, the composition lacks or has few Tmh crystals. In some embodiments, the composition comprises wherein the composition comprises, consists of, or consists essentially of:
a) about 50% by weight of soybean oil;
b) about 23.6% by weight of coconut oil;
c) about 3.3% to about 6.6% by weight of light mineral oil;
d) about 3% by weight of stearic acid;
e) about 1.1% by weight of docosanol;
f) about 1.2% by weight of hydrogenated castor oil;
g) about 3.5% by weight of cetostearyl alcohol;
h) about 2% by weight of white wax (such as beeswax);
i) about 1.5% by weight of stearyl alcohol;
j) about 2.5% by weight of myristyl alcohol; and
k) about 5% by weight of cyclomethicone.
   In some embodiments, the reduction in non-inflammatory lesion count from baseline after treatment with the composition free of both minocycline and adapalene is superior to a reduction with the composition free of adapalene, after eight weeks or less than eight weeks of treatment. In some embodiments, the reduction in non-inflammatory lesion count from baseline after treatment with a composition without minocycline is superior to treatment the composition free of both minocycline and adapalene, after eight weeks or less than eight weeks of treatment. In some embodiments, treatment with the composition free of minocycline and adapalene results in more than 45% reduction in non-inflammatory lesions after twelve weeks of treatment.

By “regular basis” it is meant a repeated or repeatable interval of time which can be, by way of illustration, a part of a day, daily, once daily, twice daily, alternative daily, alternate daily, twice weekly, trice weekly, weekly, fortnightly, monthly or some other repeated or repeatable interval for an appropriate period of time wherein a dose is to be applied. The repeated applications can be determined according to the needs of the subject and the disease or disorder. In some embodiments, as few as three repeat doses is required. In some embodiments, between 3 and 14 doses, between 14 and 28 doses, between 28 and 50 doses, between 50 and 75 doses, or between 75 and 100 doses are needed. In some embodiments, where prolonged treatment or a long period of maintenance dosing is needed, at least one hundred, at least two hundred, or at least three hundred repeat doses are needed.

By “daily dose” is intended that the dose is administered during each 24 hour period while allowing for the subject to miss a dose from time to time, and still having a treatment effect.

The term “adverse events” describes any unfavorable or unintended sign, symptom, or disease that appears or worsens in a subject after the subject has commenced using the formulation. Examples of what can be considered an adverse event (AE) include any of the following: A new illness, an exacerbation of a sign or symptom of an underlying condition or of a concomitant illness unrelated to treatment, a sign or symptom as an effect of the study drug or comparator drug. The common term for such problems is “side effects,” and used by patients and physicians.

The term “serious adverse events” describes any adverse effect that: (1) results in death; (2) is life-threatening; (3) results in hospitalization or prolongation of current hospitalization (not including hospitalization for a pre-existing condition that has not increased in severity or frequency from the subject's underlying medical condition prior to treatment); (4) results in persistent or significant disability/incapacity; (5) is a congenital anomaly/birth defect in the offspring of a subject; or (6) is an important medical event. The term “life-threatening” refers to any adverse event that, as it occurs, puts the subject at immediate risk of death, but does not refer to an adverse event that hypothetically might have caused death if it were more severe. An “important medical event” may not be immediately life-threatening or result in death or hospitalization but may be considered serious when, based upon appropriate medical judgment, may jeopardize the subject or require medical or surgical intervention to prevent one of the outcomes listed above. Examples of such medical events include allergic bronchospasm requiring intensive treatment in an emergency room or at home; blood dyscrasias or convulsions that do not result in inpatient hospitalization; or development of drug dependency or drug abuse.

In some embodiments, treatment—emergent adverse events (e.g., upper respiratory tract infection and/or dry skin) from administering a combination product disclosed herein (e.g., comprising minocycline and adapalene) are small, minimal, or absent.

In one or more embodiments, a combination product disclosed herein is well tolerated. For example, the combination product may be assessed as having “none” or “mild” for burning/stinging, itching, dryness, scaling, erythema, and/or hyperpigmentation in a treated subject.

The term “clinical failure,” as used herein, is defined as insufficient improvement or deterioration (i.e., an increase or no change in the number of lesions).

By “on average,” with reference to dosage regimes, it is intended to reflect and/or take into account human nature and that a subject may forget to apply a dose or not strictly adhere to the regime, such that even if a subject forgets a dose or does not strictly adhere to the regime it will still be considered as if the regime has been applied. For example, if a subject misses an occasional dose but does not make it up, or alternatively, if having missed a dose applies a compensatory dose on a different day, it is still counted as having complied with the dosage regime.

By “crystal fingerprint” is meant the type and distribution of crystal structures in a given formulation (whether a therapeutic composition with a therapeutic agent, or a carrier without a therapeutic agent). The crystal fingerprint may be described using one or more parameters known to one skilled in the art. Exemplary parameters include number and type of crystals in a given area when viewed in a light microscope, cross-sectional diameter, cross-sectional area, shape, melting temperature, enthalpy, flow point temperature, certain bands from X-ray crystallography, X-ray diffraction pattern, Raman spectroscopy, space group, and/or certain points of inflection or shift seen from DSC, and/or FTIR (Fourier Transform Infrared Spectroscopy) parameters. FTIR parameters may include wavenumber, band intensity, and band sharpness.

FTIR analysis may be used to identify and characterize functional groups and changes in the interactions (i.e., hydrogen bonds) that occur during molecular self-assembly (i.e., crystallization, polymerization), molecular disassembly (i.e., melting) or compounds' decomposition. Hydrogenated castor oil (HCO) is mainly composed of 12-hydroxyl stearic acid molecules (i.e., trihydroxystearin), capable of molecular self-assembly through hydrogen bonding developed by the hydroxyl groups of different 12-hydroxyl stearic acid molecules.

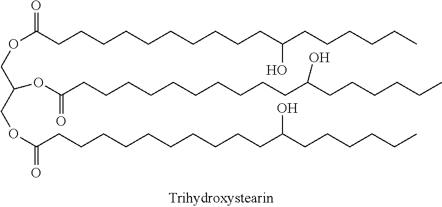 [SMILES: CCCCCCC(O)CCCCCCCCCCC(=O)OCC(COC(=O)CCCCCCCCCCC(O)CCCCCC)OC(=O)CCCCCCCCCCC(O)CCCCCC]

Without being bound by theory, FTIR may measure the vibration of O and H molecules and serves as an indicator of hydrogen bond strength. In some embodiments, a waveband observed in FTIR between 3000-3400 cm⁻¹ indicates hydrogen bonding stretching vibration in a composition. In some embodiments, light is shone at crystals and stronger H-bonds are indicated by a red shift to a lower frequency of waveband. This may be quantified by measuring the absorption of an FTIR waveband, e.g., by comparing wavelengths and/or calculating the area under the curve from the FTIR waveband absorption measurement. A larger area indicates stronger H-bonds. A higher intensity band or a shift to a lower frequency may also indicate stronger H-bonds. In some embodiments, multiple FTIR wavebands may be considered. A lower wavenumber, a higher intensity band, and/or a band of lower frequency (e.g., a band between about 3350-3305 cm⁻¹) may be observed for a composition prepared with a holding process than for a composition prepared without a holding process, indicating stronger H-bonds.

In some embodiments, a comparison of a formulation comprising oils (e.g., coconut oil, soybean oil, mineral oil, and/or cyclomethicone) and HCO (e.g., 1.2% HCO) reveals the presence of hydrogen bonds (e.g., a waveband at the 3350-3305 cm⁻¹ interval) when HCO is present.

In various embodiments, an improvement in H-bond strength is observed with Tmh crystals.

In one or more embodiments, the frequency of the bands within the composition is indicated by the wavenumber, as measured by FTIR. In one or more embodiments, the composition prepared by a holding step has lower frequency, as measured by FTIR, than that of a composition prepared without a holding step. In one or more embodiments, the composition prepared by a holding step has lower frequency, as measured by FTIR, than that of a composition prepared by a continuous heating-cooling process. In one or more embodiments, the composition having Tmh crystals has lower frequency, as measured by FTIR, than that of a composition lacking Tmh crystals or a composition having a smaller percentage by area of Tmh crystals as compared to the percentage of spherulite or plate crystals in the composition.

In one or more embodiments, the absorbance of the hydrogen bond vibrational bands in the composition is indicated by the wavenumber of the composition, as measured by FTIR. In one or more embodiments, the composition prepared by a holding step has multiple wavebands as measured by FTIR, where at least one band absorbs at a lower wavenumber as measured by FTIR, as compared to a composition prepared without a holding step. In one or more embodiments this band is above 3300 cm⁻¹, e.g., when measured at 25° C. or 50° C. In some embodiments this band is about 3000 cm⁻¹. In some embodiments this band is about 3000-3600 cm⁻¹. In some embodiments this band is about 3300 cm⁻¹. In one or more embodiments, the composition prepared by a holding step has a first band which absorbs at a lower wavenumber and a second band which absorbs at the same wavenumber, as measured by FTIR, as compared to a composition prepared without a holding step. In one or more embodiments the second band is below about 3000-3600 cm⁻¹, e.g., below 3300 cm⁻¹. In some embodiments the second band is about 3200 cm⁻¹. In one or more embodiments, the composition prepared by a holding step has a first band which absorbs at lower wavenumber and a second band which absorbs at the same wavenumber, as measured by FTIR, as compared to a composition prepared by a continuous heating-cooling process. In one or more embodiments, the composition has a first band which absorbs at lower wavenumber and a second band which absorbs at the same wavenumber, as measured by FTIR, as that of a composition lacking Tmh crystals or a composition having a smaller percentage by area of Tmh crystals as compared to the percentage of spherulite or plate crystals in the composition. In one or more embodiments, the composition has a first band having a wavenumber of about 3301-3312 cm⁻¹ when measured at 25° C., as measured by FTIR. In one or more embodiments, the composition has a band having a wavenumber of about 3320-3324 cm⁻¹ when measured at 50° C., as measured by FTIR.

In one or more embodiments, the intensity of a band within the composition is indicated by the area under the band's peak, as measured by FTIR. In one or more embodiments, the intensity of the band in a composition prepared by a holding step is higher, as measured by FTIR, than those of a composition prepared without a hold step. In some embodiments the band is band 1, in some embodiments the band is band 2 and in some embodiments the band is band 1 and 2. In one or more embodiments, the intensity of the bands in a composition prepared by a holding step is higher, as measured by FTIR, than those of a composition prepared without a hold step. In one or more embodiments, the intensity of these bands in a composition prepared by a holding step is higher, as measured by FTIR than that of a composition prepared by a continuous heating-cooling process. In one or more embodiments, the intensity of these bands in a composition prepared by a holding step is higher, as measured by FTIR than that of a lacking Tmh crystal or a composition having a smaller percentage by area of Tmh crystals as compared to the percentage of spherulite or plate crystals in the composition.

In one or more embodiments, a composition prepared by a holding step has more hydrogen bonds between crystals, as measured by FTIR, than that of a composition prepared without a holding step. In one or more embodiments, a composition prepared by a holding step has more hydrogen bonds between crystals, as measured by FTIR, than that of a composition prepared by a continuous heating-cooling process. In one or more embodiments, the composition has more hydrogen bonds between crystals, as measured by FTIR, than that of a composition lacking Tmh crystals or a composition having a smaller percentage by area of Tmh crystals as compared to the percentage of spherulite or plate crystals in the composition.

In one or more embodiments, a change in intensity, as measured by small x-ray scattering, indicates a change in crystal structure. In one or more embodiments, a higher intensity, as measured by small x-ray scattering, indicates crystals of higher order and/or crystals that are more closely packed. In one or more embodiments, the composition prepared by a holding step shows higher intensity, as measured by small x-ray scattering, compared to a composition prepared without a holding step. In one or more embodiments, the composition prepared by a holding step shows higher intensity, as measured by small x-ray scattering, compared to a composition prepared by a continuous heating-cooling process. In one or more embodiments, the composition prepared by a holding step has crystals of high order. In one or more embodiments, the composition prepared by a holding step has crystals that are closely packed. In one or more embodiments, the composition prepared by a holding step has crystals of higher order, as measured by small x-ray scattering, compared to crystals of a composition prepared by a continuous heating-cooling process. In one or more embodiments, the composition prepared by a holding step has crystals that are more closely packed, as measured by small x-ray scattering, compared to crystals of a composition prepared by a continuous heating-cooling process.

In one or more embodiments, a change in wide angle x-ray scattering pattern, indicates a change in crystal polymorph. In one or more embodiments, a composition prepared by a holding step shows no change in a wide angle x-ray scattering pattern as compared to a composition prepared by a continuous heating-cooling process. In one or more embodiments, a composition prepared by a holding step has no change in crystal polymorph as compared to a composition prepared by a continuous heating-cooling process.

In one or more embodiments, the composition prepared by a holding step shows a change in a wide angle x-ray scattering pattern as compared to a composition prepared by a continuous heating-cooling process. In one or more embodiments, a composition prepared by a holding step has a change in crystal polymorph as compared to a composition prepared by a continuous heating-cooling process.

In some embodiments, a crystal fingerprint is characterized by evaluating the average cross-sectional diameter in the longest dimension of one or more crystals in a formulation, e.g., to identify Tmh crystals in the formulation, or to identify plate and/or spherulite crystals in the formulation. In some embodiments, a crystal fingerprint is characterized by evaluating the average cross-sectional area of one or more crystals in a formulation, e.g., to identify Tmh crystals in the formulation, or to identify plate and/or spherulite crystals in the formulation. In some embodiments, the number or percentage of Tmh crystals in a sample, or the density of Tmh crystals (e.g., the percentage of Tmh crystals in a sample relative to the percentage of other crystals such as spherulites) are used to measure the fingerprint. In some embodiments, a crystal fingerprint is characterized by the percentage of Tmh crystals as compared to the percentage of spherulite or plate crystals in the composition. In some embodiments, a crystal fingerprint is characterized by the melting temperature of the crystals in the composition. In some embodiments, a crystal fingerprint is characterized by the percentage and/or distribution of different types of crystals in the composition. In some embodiments, a crystal fingerprint is characterized by the X-ray powder diffraction pattern of the crystals in the composition. In some embodiments, a crystal fingerprint is characterized by hydrogen bonding. In some embodiments, a composition prepared using a holding step may exhibit a crystal finger print with stronger hydrogen bonds than seen in a composition prepared without a hold step (e.g., it may exhibit a shift to lower frequency wave bands and/or higher intensity wave bands, as measured by FTIR).

As used herein, the term “spherulites” describes spheroidal crystal structures composed of acicular or fibrous crystals grouped around a central point that have an average area of about 20-30 μm² e.g., 23-25 μm².

As used herein, the terms “plates,” “platelets,” or “plate-like structures” describe crystal units in which the structure is larger in a two-dimensional plane as compared to a one-dimensional plane and has an average area of about 10-20 μm² e.g., 12-19 μm². In some embodiments of the present disclosure, spherulites and plates, or a higher percentage of spherulites and plates, are prepared in a continuous heating-cooling process than when using a hold step. In some embodiments, the average area of a plate prepared in a holding process e.g., about 14-20 μm² is larger than that prepared in a continuous heating-cooling process e.g., about 10-14 μm².

As used herein, the term “nonuniform crystals” and crystals with “non-uniform structures” describe crystals that have a nonuniform crystal structure, i.e., do not have a defined crystal pattern, and have higher melting temperatures. These may also be referred to as “irregular crystals” or “tangled fibers.” These are observed as large structural units of an irregular form, e.g., those with an average largest cross-sectional area of 40-150 μm² on average, 50-150 μm² on average e.g., about 50-130 μm² on average, e.g., about 100-122 μm². e.g., of about 50-80 μm² on average, e.g., about 50-70 μm² on average, e.g., about 55-70 μm² on average e.g., about 61-63 μm² on average. Nonuniform crystals include those that cannot be classified as spherulites or plates. In some embodiments, the nonuniform crystals disclosed herein are tangled fibers. In some embodiments, tangled fibers exhibit a larger widest cross-sectional area and lack the regularity of the cross-sectional area observed in a spherulite or plate crystal.

As used herein, the term “Tmh crystal” describes crystals having a phase transition temperature T_{M4} of about 66-80° C., as measured by DSC, and are formed during a mixing and cooling process employing a hold step. Tmh crystals are observed as having a nonuniform structure. In some embodiments, the T_{M4} of a Tmh crystal may shift upwards when adding active agents such as adapalene and/or minocycline to a formulation. In some embodiments, the Tmh crystals have stronger interactions between unit cells, e.g., the molecular forces (e.g., Van der Waals, hydrogen bonds etc.) participating in the formation of the crystal lattice are stronger. In one or more embodiments the stronger interaction results from an increased number of such interactions or bonds. In one or more embodiments, the Tmh crystals disclosed herein are structures with more Van Der Waals bonds. In one or more embodiments, the Tmh crystals disclosed herein are structures with more hydrogen bonds. In some embodiments, the Tmh crystals have stronger intermolecular interactions. In some embodiments, the Tmh crystals have stronger intra-molecular interactions. In some embodiments the crystal structure discloses a higher intensity when measures by x-ray crystallography. In some embodiments, a formulation comprising Tmh crystals exhibits a DSC pattern comprising a melting temperature above 68° C., e.g., a melting temperature of about 68-73° C., e.g., about 68-72° C., e.g., about 68-69° C.. In some embodiments, a higher percentage and/or density of Tmh crystals are prepared when using a holding process. In one or more embodiments, Tmh crystals provide a formulation comprising aT_{M4} as measured by DSC that is about 3° C. or about 4° C., or about 5° C., or about 6° C. higher than that of a formulation prepared in a continuous heating-cooling process. In some embodiments a formulation comprising Tmh crystals has an SRS (raman) spectra in the range of about 1400-1500 cm⁻¹, with a peak at about 1446 cm⁻¹ and having one or two shoulders at about 1465 cm⁻¹ and/or at about 1425 cm⁻¹.

In some embodiments, the differences observed between the crystals prepared using a continuous cooling process and those of the holding process described herein, e.g., those observed in a placebo formulation and/or a formulation comprising an active such as minocycline and/or adapelen, include:

| Characteristic | Continuous process | Holding process |
|---|---|---|
| Morphology of crystals | Majority of spherulites | some, e.g., a majority of Tmh |
| | | crystals |
| Average size of crystals | Spherulites of 20-35 μm² | Tmh crystals of about 50-150 μm² |
| Average DSC T_{M4} peak | <67° C. | 68-74° C. |
| temperature | | |
| Average FTIR Band 1 | In range 3316-3325 cm⁻¹ | In range 3300-3314 cm⁻¹ |

In some embodiments, similar properties are expected in placebo formulations and those having an API, e.g., minocycline, e.g., minocycline hydrochloride, when prepared using a holding step.

As used herein, “shakability” refers to the degree to which the user is able to feel or hear the presence of the foamable composition when the filled pressurized canister is shaken. Shaking is done with mild to normal force without vigorous or excessive force. When the user cannot sense the motion of the contents during shaking the foamable composition may be considered to be non-shakable. When the user can moderately sense the motion of the contents during the shaking, the foamable composition is considered moderately shakable. When the contents are flowable during shaking, the product is considered shakable.

Unique Crystals and/or Crystal Fingerprint

In one or more embodiments, the composition and/or foamable composition comprises crystals with a crystal fingerprint (including numbers, sizes, types and distribution etc.) that have not been described previously in the literature. In some embodiments the unique crystals have a higher order and or a nonuniform structure. In one or more embodiments the presence of these unique crystals and/or said crystal fingerprint may alter the properties of the composition, and or foamable composition e.g., by improving one or more characteristics including, usability, melting temperature, the ability to facilitate sebum liquification, fluidity, shakability, stability and storability. In one or more embodiments, the crystals in the composition and/or foamable composition helps reduce or eliminate a need for refrigeration or cool storage. In one or more embodiments, the crystals and/or crystal fingerprint in the compositions and/or foamable compositions can improve penetration, delivery and/or distribution into skin, mucosa or wall of a body cavity of one or more active pharmaceutical agents (e.g., MCH and ADP). In some embodiments, the crystals increase the softening, breakdown, and/or dissolution of sebum. In one or more embodiments each of these improvements is achieved without compromising, impacting on, or reducing the chemical stability of the unstable active ingredients in the improved formulations.

In one or more embodiments the Tmh crystals in the formulations disclosed herein exhibit a fingerprint comprising Tmh crystals distributed throughout the formulation. In some embodiments the Tmh crystals are distributed homogeneously throughout the formulation (e.g., as determined by observing a sample of a formulation under a light microscope to observe the presence of crystals throughout the sample). In some embodiments the Tmh crystal forms appear to cluster within the composition, e.g., as determined by observing a sample of a formulation under a light microscope to observe groupings of increased crystal density at locations throughout the sample, as compared to other locations in the sample or as compared to a sample from a formulation prepared without a hold step. In some embodiments, these clusters are themselves homogeneously distributed throughout the formulation (e.g., as determined by observing a sample of a formulation under a light microscope to observe clusters of crystal density dispersed evenly throughout the sample). These Tmh crystals and/or the crystal fingerprints described herein can be obtained from the compositions and/or foamable compositions and/or methods described herein and as described more particularly below.

### Compositions

Gel or foam compositions comprising, e.g., tetracycline antibiotic are described in U.S. Patent Application Publication Nos. 2014/0121188 and 2013/0225536, which are herein incorporated by reference in their entirety.

In one or more embodiments there is provided a hydrophobic gel or foamable composition comprising hydrogenated castor oil, wherein the hydrogenated castor oil is present in an amount effective to form a stable foamable formulation. In one or more embodiments there is provided a hydrophobic gel or foamable composition comprising a tetracycline antibiotic and hydrogenated castor oil, wherein the hydrogenated castor oil is present in an amount effective to form a stable foamable formulation. In one or more embodiments there is provided a hydrophobic gel or foamable composition comprising a tetracycline antibiotic and/or a retinoid, and hydrogenated castor oil, wherein the hydrogenated castor oil is present in an amount effective to form a stable foamable formulation. In some embodiments, such foamable composition are for use in treating a skin disorder, e.g., acne conglobate, acne vulgaris or rosacea, in a human subject suffering therefrom. In some embodiments, treatment methods using the foamable compositions described herein comprise topically administering the composition to the human subject in a sufficient amount and for a sufficient time to achieve efficacy, e.g., decrease the number of inflammatory or non-inflammatory lesions or achieve IGA treatment success.

### Hydrogenated Castor Oil

Without being bound by theory, hydrogenated castor oil is a saturated vegetable oil wax which may allow hydrophobic and hydrophilic components of the foamable compositions or hydrophobic gels described herein to form a homogenous or uniform composition. Foamable compositions comprising hydrogenated castor oil and produced by the methods described herein may provide for surprisingly improved stability, serving as a suitable carrier for active agents that may be easily degraded, e.g., a tetracycline antibiotic such as minocycline HCl and/or a retinoid such as adapalene. The methods and compositions described herein may produce a foamable composition that has superior shakability and microcrystal structures.

In some embodiments, foamable composition are provided, comprising hydrogenated castor oil, wherein the hydrogenated castor oil is present in an amount effective to form a stable foamable formulation. In some embodiments, the foamable composition comprises about 1% to about 3% hydrogenated castor oil. In some embodiments, the foamable composition comprises about 1% to about 2% hydrogenated castor oil. In some embodiments, the foamable composition comprises about 1% to about 1.5% hydrogenated castor oil. In some embodiments, the foamable composition comprises about 1.1% to about 1.3% hydrogenated castor oil. In some embodiments, the foamable composition comprises about 2% hydrogenated castor oil. In some embodiments, the foamable composition comprises about 1.2% hydrogenated castor oil. In some embodiments, the foamable composition comprises about 1.9% to about 2.1% hydrogenated castor oil.

Without being bound by theory, using a cooling hold step as described herein with a formulation comprising hydrogenated castor oil, e.g., to produce Tmh crystals in the formulation, may surprisingly prevent a reduction in Tm when adding hydrogenated castor oil to, e.g., a foamable formulation (compare, e.g., the drop in Tm reported in Example 2 when preparing formulations without a hold step).

### Therapeutic Agents

In some embodiments the carrier has a therapeutic effect. In one or more embodiments the carrier comprises one or more therapeutic agents. In one or more embodiments the therapeutic agent is pharmaceutical active agent. In one or more embodiments it is present in a therapeutically effective amount for topical application to skin, mucosa or body cavity surface as would be appreciated by a physician skilled in such topical application. In one or more embodiments the therapeutic agent is a cosmetic or cosmeceutical active agent. In one or more embodiments it is present in a therapeutically effective amount for topical application to skin, mucosa or body cavity surface as would be appreciated by a physician skilled in such topical application.

As used herein, the term “cosmeceutical agent” refers to a cosmetic agent that has one or more medicinal, therapeutic, and/or drug-like benefits. Non-limiting examples include alpha hydroxy acids (AHAs), beta hydroxy acids (BHAs) and polyhydroxy acids (PHAs), vitamins such as vitamin A, vitamin B, vitamin B3, vitamin C, vitamin D, vitamin D3, vitamin E, vitamin derivatives, lipoic acid, salicylic acid, keratolytic agents, peeling agents, depigmenting (or bleaching) agents such as hydroquinone and kojic acid, botanical and marine extracts, UV filters, UV absorbers, lactic acid, retinol, retinoid and nicotinamide. In some embodiments, a cosmeceutical agent may be a pharmaceutical agent that is used at low doses to provide one or more benefits to skin and mucosa. In one or more embodiments, a cosmeceutical agent can manipulate and/or modulate the biological function of the skin, e.g., improve appearance of the skin, such as skin tone, texture, clarity and/or wrinkles by delivering nutrients essential for healthy skin.

In one or more embodiments, the active agent comprises an antibacterial agent. In certain embodiments, the antibacterial active agent is a tetracycline antibiotic. In one or more embodiments, the tetracycline antibiotic is oxytetracycline, demeclocycline, doxycycline, lymecycline, meclocycline, methacycline, minocycline, rolitetracycline, chlorotetracycline, tigecycline, or a mixture of two or more thereof. In one or more embodiments, the tetracycline is minocycline or a salt thereof. In one or more embodiments, the tetracycline is minocycline hydrochloride.

In some embodiments, a formulation disclosed herein comprises minocycline, e.g., about 1.5-3%, e.g., about 1.5% or about 3%. In some embodiments, the minocycline is present as minocycline hydrochloride. In some embodiments, the amount of minocycline hydrochloride in a formulation is adjusted to correspond to a total amount of minocycline of about 1.5-3%. For instance, about 1.29-3.58% of minocycline hydrochloride may be used to provide about 1.50-3.00% of minocycline.

In one or more embodiments, the tetracycline is doxycycline or a salt thereof. In one or more embodiments, the tetracycline is doxycycline hyclate. In one or more embodiments, the tetracycline is doxycycline monohydrate. In one or more embodiments, the tetracycline antibiotic is present in a free base form, a hydrate form, a salt form, or a complex form. In one or more embodiments, the tetracycline is soluble or is partially soluble in the composition. In one or more embodiments, a part of the tetracycline is suspended in the composition. In one or more embodiments, properties or uses discovered for doxycycline or minocycline compositions can be applicable to other tetracycline antibiotic compositions.

In one or more embodiments, a composition provided herein comprises one or more active agents selected from, but not limited to, one or more of lysine, an active herbal extract, an acaricides, an age spot and keratose removing agent, an allergen, an alpha hydroxyl acid, an analgesic agent, an antiacne agent, an antiallergic agent, an antiaging agent, an antibacterial agent, an antibiotic, an antiburn agent, an anticancer agent, an antidandruff agent, an antidepressant, an antidermatitis agent, an antiedemic anent, an antifungal agent, an antihistamine, an antihelminth agent, an antihyperkeratolyte agent, an anti-infective agent, an antiinflammatory agent, an antiirritant, an antilipemic agent, an antimicrobial agent, an antimycotic agent, an antioxidant, an antiparasitic agent, an antiproliferative agent, an antipruritic agent, an antipsoriatic agent, an antirosacea agent, an antiseborrheic agent, an antiseptic agent, an antiswelling agent, an antiviral agent, an anti-wart agent, an anti-wrinkle agent, an antiyeast agents, an astringent, a beta-hydroxy acid, benzoyl peroxide, a topical cardiovascular agent, a chemotherapeutic agent, a corticosteroid, an immunogenic substance, a dicarboxylic acid, a disinfectant, a fungicide, a hair growth regulator, a haptene, a hormone, a hydroxy acid, an immunosuppressant, an immunoregulating agent, an immunomodulator, an insecticide, an insect repellent, a keratolytic agent, a lactam, a local anesthetic agent, a lubricating agent, a masking agent, a metals, a metal oxide, a mitocide, a neuropeptide, a non-steroidal anti-inflammatory agent, an oxidizing agent, a pediculicide, a peptide, a protein, a photodynamic therapy agent, a radical scavenger, a refatting agent, a retinoid, a sanative, a scabicide, a self-tanning agent, a skin protective agent, a skin whitening agent, a steroid, a steroid hormone, a vasoconstrictor, a vasodilator, a vitamin, a vitamin A, a vitamin A derivative, a vitamin B, a vitamin B derivative, a vitamin C, a vitamin C derivative, a vitamin D, a vitamin D derivative, a vitamin D analog, a vitamin F, a vitamin F derivative, a vitamin K, a vitamin K derivative, a wound healing agent and a wart remover, an androgen, an anti-hyperkeratosis agent, an estrogen, an immunostimulent, a pesticide, a progesterone, an azole, metronidazole, a sedative, a vaso-active agent and mixtures of any two or more active agents.

In some embodiments, a composition provided herein comprises a tetracycline antibiotic and at least one additional active agent, for example, a tertracycline antibiotic and a retinoid, a tetracycline antibiotic and a steroid, or a tetracycline antibiotic and a retinoid and a steroid. In one or more embodiments the retinoid is adapalene or tazarotene.

In one or more embodiments, the composition, and/or foamable composition can be a placebo (i.e., a vehicle or a carrier) composition. In one or more embodiments, the composition, and/or foamable composition can be substantially free of an active agent. In some embodiments, the composition, and/or foamable composition is essentially free of active agent. In some embodiments, the composition, and/or foamable composition can be free of active agent. The composition, and/or foamable composition is suitable for use in the manufacture of a medicament.

In some embodiments, the methods of preparing the compositions described herein surprisingly improve the properties of a placebo composition, e.g., stability, shakability, flowability etc. In some embodiments, a placebo composition prepared by a holding process improves the properties of the composition, e.g., restoration of the enthalpy in the placebo to match that seen in a formulation with active agents.

### Combination of Active Agents

Several disorders involve a combination of more than one etiological factor; and therefore, the use of more than one active agent is advantageous. For example, psoriasis involves excessive cell proliferation and inadequate cell differentiation as well as inflammation. Atopic dermatitis involves keratinocyte growth abnormality, skin dryness and inflammation. Bacterial, fungal and viral infections involve pathogen colonization at the affected site and inflammation. Hence, in many cases, the inclusion of a combination of active agents in the pharmaceutical composition can be desirable. Thus, in one or more embodiments, the composition includes at least two active agents, in a therapeutically effective concentration. In some embodiments, the composition includes at least three active agents, in a therapeutically effective concentration. In some embodiments, the composition includes at least four active agents, in a therapeutically effective concentration. In some embodiments, the composition includes at least five active agents, in a therapeutically effective concentration.

In one or more embodiments, a combination of any two or more of an antibacterial, an anti-inflammatory, an antifungal, an antiviral agent and an immunomodulating agent is contemplated.

In one or more embodiments, there is provided a composition comprising a combination of an antibiotic and at least one additional active agent selected from the group consisting of a vasoconstrictor, an α2 adrenergic agonist, a tyrosine kinase inhibitor, a VEGF inhibitor, a JAK inhibitor, a dicarboxylic acid, a serine protease inhibitor, and an aldosterone receptor inhibitor.

In one or more embodiments, there is provided a composition comprising a combination of a tetracycline antibiotic and at least one additional active agent selected from the group consisting of brimonidine, pazopanib, tofacitinib, azelaic acid, aminocaproic acid, and spironolactone.

In one or more embodiments, there is provided a composition comprising a combination of a minocycline and at least one additional active agent selected from the group consisting of brimonidine, pazopanib, tofacitinib, azelaic acid, aminocaproic acid, and spironolactone.

In one or more embodiments, there is provided a composition comprising a combination of a doxycycline and at least one additional active agent selected from the group consisting of brimonidine, pazopanib, tofacitinib, azelaic acid, aminocaproic acid, and spironolactone.

In one or more embodiments, there is provided a composition in which the composition further comprises at least one additional active agent selected from the group consisting of an antibiotic agent, an anti-inflammatory agent, a steroidal anti-inflammatory agent, an immunosuppressive agent, an immunomodulator, an immunoregulating agent, a hormonal agent, an androgen, an estrogen, a prostaglandin, an antiandrogen agent, a testosterone inhibitor, a dihydrotestosterone inhibitor, antibacterial agent, an antifungal agent, an antiviral agent, an antiparasitic agent, antimicrobial, a retinoid, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, a keratolytic agent, urea, a urea derivative, an alpha hydroxyl acid, lactic acid, glycolic acid, a beta-hydroxy acid, a protein, a peptide, a neuropeptide, an allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, benzoyl chloride, calcium hypochlorite, magnesium hypochlorite, an anti-wrinkle agent, a radical scavenger, a metal, silver, a metal oxide, titanium dioxide, zinc oxide, zirconium oxide, iron oxide, silicone oxide, an organo-metallic compound, and organo-boron compound, an organo-beryllium compound, a tellurium compound, talc, carbon, an anti-wrinkle agent, a skin whitening agent, a skin protective agent, a masking agent, an anti-wart agent, a refatting agent, a lubricating agent, and mixtures thereof. In one or more embodiments where a derivative of an active agent is mentioned it is intended to provide derivatives that are known and/or are used for and/or contemplated for treatment of skin, or mucosa, or a body cavity surface or wall.

In one or more embodiments the addition of at least one additional active agent is optional.

Wherever a specific active agent is used herein, it can be substituted by another form of the same active agent. For example, in one or more embodiments, minocycline hydrochloride can be substituted by another form of minocycline, and likewise in one or more embodiments, doxycycline hyclate can be substituted by another form of doxycycline. The term “form” can include, for example, salts, hydrates, crystals, polymorphs, enantiomers, isomers, ions, complexes, and the like. In one or more embodiments, the active agent can be in the form of a salt, a hydrate, a crystal, one or more polymorphs, one or more enantiomers, an isomer, an ion, a complex, or any other pharmaceutically acceptable form.

In one or more embodiments, a tetracycline antibiotic is the sole active ingredient present in the composition. In one or more embodiments, a minocycline is the sole active ingredient present in the composition. In one or more embodiments, a doxycycline is the sole active ingredient present in the composition. In one or more embodiments, minocycline and doxycycline are used in combination.

In one or more embodiments, a combination of any two or more of a minocycline, doxycycline, tetracycline antibiotic, steroids, corticosteroids, vitamin K, topical anesthetics, antipruritic agents, antihistamines, pramoxine, lidocaine, quaternary lidocaine derivatives, quaternary ammonium derivatives of anesthetic drugs, pimecrolimus, tarcolimus, retinoids, and benzoyl peroxide is contemplated.

In one or more embodiments, quaternary ammonium derivatives of anesthetic drugs include, for example, quaternary lidocaine derivatives, N-methyl lidocaine, N,N-dimethyl prilocaine, N,N,N-trimethyl tocainide, N-methyl etidocaine, N-methyl ropivacaine, N-methyl bupivacaine, N-methyl levobupivacaine, N-methyl mepivacaine, QX314, and Q222, and as are described in US2012/0172429, which is incorporated by reference. In one or more embodiments the effect of these quaternary ammonium derivatives of anesthetic drugs is associated with TRPA1, TRPM8, P2X(2/3) or TRPV1 channels and receptors. In one or more embodiments they bind to receptors on the side of the channels which is internal.

In one or more embodiments, a combination of any two or more of tetracycline antibiotics, a retinoid, azelaic acid and benzoyl peroxide is contemplated.

In one or more embodiments, a combination of a tetracycline antibiotic, and a JAK inhibitor (e.g., baricitinib or tofacitinib) is contemplated.

In one or more embodiments, a combination of any two or more of benzoyl peroxide, antibiotics, tetracycline antibiotic, retinoids, antiseborrheic medications, anti-androgen medications, hormonal treatments, lactic acid, urea, petrolatum, emollients, salicylic acid, alpha hydroxy acid, azelaic acid, nicotinamide, and a keratolytic agent is contemplated.

In one or more embodiments, the tetracycline is in combination with of one or more of an antihistamine, a corticosteroid, doxepin, or adapalene.

In one or more embodiments, the concentration of the additional active agent is in a range between about 0.1% to about 15% by weight (e.g., about 0.1% to about 14% by weight, about 0.1% to about 12% by weight about 0.1% to about 10% by weight about 0.1% to about 8% by weight, or about 0.1% to about 5% by weight, or about 0.1% to about 3% by weight, or about 0.1% to about 2% by weight, or about 0.1% to about 1% by weight, or about 0.1% to about 0.75% by weight, or about 0.1% to about 0.5% by weight, or about 0.1% to about 0.25% by weight, or about 0.25% to about 10% by weight, or about 0.5% to about 10% by weight, or about 1% to about 10% by weight, or about 2% to about 10% by weight, or about 4% to about 10% by weight, or about 6% to about 10% by weight, or about 7% to about 10% by weight, or about 8% to about 10% by weight, or about 0.5% to about 2.0% by weight, or about 0.75% to about 1.5% by weight, or about 1% to about 3% by weight, or about 1% to about 4% by weight, or about 2% to about 6% by weight). In some embodiments, the concentration of the additional active agent is about or at least about 0.05% by weight, is about or is at least about 0.1% by weight, is about or at least about 0.2% by weight, is about or at least about 0.3% by weight, is about or at least about 0.4% by weight, is about or at least about 0.5% by weight, is about or at least about 0.6% by weight, is about or at least about 0.8% by weight or at least about 1% by weight, is about or at least about 1.5% by weight, is about or is at least about 2% by weight, is about or at least about 2.5% by weight, is about or at least about 3% by weight, is about or at least about 3.5% by weight, is about or at least about 4% by weight, is about or at least about 4.5% by weight, is about or at least about 5% by weight or at least about 5.5% by weight, agent is about or at least about 5.5% by weight, is about or is at least about 6% by weight, is about or at least about 6.5% by weight, is about or at least about 7% by weight, is about or at least about 7.5% by weight, is about or at least about 8% by weight, is about or at least about 8.5% by weight, is about or at least about 9% by weight or at least about 10% by weight, is about or at least about 11% by weight, is about or at least about 12% by weight, is about or at least about 13% by weight, is about or at least about 14% by weight, is about or at least about 15% by weight; or can be a range between any two figures listed in this paragraph, such as, 0.05% to about 0.8%.

### Tetracycline Antibiotic

Any unstable topical therapeutic or active agent, e.g. a tetracycline antibiotic, may be used with the formulations disclosed herein. Similarly, any stable topical therapeutic or active agent may be used on their own or in combination with any unstable active agent in the formulations disclosed herein. In some embodiments, any tetracycline antibiotic known to one of skilled in the art can be used with the formulations disclosed herein. Examples of a tetracycline antibiotic include, for example, but not limited to tetracycline, oxytetracycline, demeclocycline, doxycycline, lymecycline, meclocycline, methacycline, minocycline, rolitetracycline, chlorotetracycline, and tigecycline.

In certain embodiments, the tetracycline is a minocycline or doxycycline. According to one or more embodiments, the tetracycline is minocycline. According to one or more embodiments, the minocycline is minocycline hydrochloride (minocycline HCl, or MCH). In some embodiments, MCH is a yellow crystalline powder that is sparingly soluble in water, slightly soluble in alcohol, and/or practically insoluble in chloroform and in ether. MCH may be quickly degraded when dissolved in water. Without being bound by any theory, degradation of small amounts of dissolved MCH can hasten the dissolution of more MCH which in turn degrade, and which may drive a cycle of rapid degradation.

Minocycline and MCH are known to be highly sensitive to moisture, air and light and undergo rapid degradation. The presence of even small amounts of water can cause degradation. Compatible excipients have become incompatible in the presence of water. Addition of antioxidants did not alter this result. Therefore, storage of formulations in airtight/lighttight sealed containers or tubes or foamable formulations in airtight/lighttight sealed containers under pressure with propellant can contribute to preserving stability, subject to selection of compatible canisters and accessories. Likewise, production and/or filing under vacuum in an oxygen free environment and purging with nitrogen can help.

It was unexpectedly demonstrated that foamable compositions comprising hydrogenated castor oil and a tetracycline antibiotic, e.g., minocycline, when applied topically, provide therapeutic benefits for skin disorders, e.g., acne conglobate, acne vulgaris or rosacea. Foamable compositions disclosed herein comprising hydrogenated castor oil, e.g., at about 0.1-3%, e.g., about 1.2%, may provide for surprisingly improved stability, allowing such compositions to be packaged in aerosol containers. The ease of use, with once daily dosing, as well as its broad spectrum of activity, early onset, the low level of adverse events and the rapid reduction in the number of lesions make it an attractive choice and a potentially valuable medication for the treatment of acute bacterial skin infections.

Examples of bacterial infections that can be effectively treated by topical tetracycline antibiotics include, but not limited to, cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma, disorders of hair follicles and sebaceous glands, acne, impetigo, rosacea, perioral dermatitis, hypertrichosis (hirsutism), alopecia, including male pattern baldness, alopecia greata, alopecia universalis and alopecia totalis, pseudofolliculitis barbae, and keratinous cyst. For example, rosacea involves papules and pustules, which can be treated with an antibiotic agent, as well as erythema, telangiectasia, and redness, which partially respond to treatment with an antibiotic agent. Similarly, acne vulgaris involves papules, pustules, open or closed comedones, and nodules, which can be treated with an antibiotic agent.

In one or more embodiments, the tetracycline antibiotic has some hydrophobic/lipophilic properties.

In one or more embodiments, the Log of the distribution constant of the tetracycline antibiotic at pH 7.0 (buffer/chloroform) is equal to or less than about 0.2.

In one or more embodiments, tetracycline antibiotic forms suitable for use according to the methods and compositions of the present disclosure include, but are not limited to, a free base form, a hydrate form, a salt form, a chelate complex form or a coordination complex form.

In one or more embodiments, the tetracycline antibiotic does not comprise a hydroxyl group at carbons 5, 6, and 7.

In one or more embodiments, the tetracycline antibiotic comprises or is selected from the group consisting of a minocycline and a doxycycline. In some embodiments, the tetracycline antibiotic is a minocycline. In some embodiments, the concentration of the tetracycline antibiotic for topical application e.g. minocycline is in a range between about 0.1% to about 12% by weight (e.g., about 0.1% to about 11% by weight, about 0.1% to about 10% by weight, about 0.1% to about 9% by weight, about 0.1% to about 8% by weight, about 0.1% to about 7% by weight, about 0.1% to about 6% by weight, about 0.1% to about 5% by weight, about 0.1% to about 4% by weight about 0.1% to about 3% by weight, about 0.1% to about 2% by weight, about 0.1% to about 1% by weight, about 0.1% to about 0.75% by weight, about 0.1% to about 0.5% by weight, about 0.1% to about 0.25% by weight, about 0.25% to about 10% by weight, about 0.5% to about 10% by weight, about 0.5% to about 5% by weight, about 0.5% to about 4% by weight, about 0.5% to about 3% by weight, about 1% to about 10% by weight, about 2% to about 10% by weight, about 4% to about 10% by weight, about 6% to about 10% by weight, about 7% to about 10% by weight, about 8% to about 10% by weight, about 0.5% to about 2.0% by weight, about 0.75% to about 1.5% by weight, about 1% to about 3% by weight, about 1% to about 4% by weight, and about 2% to about 6% by weight). In some embodiments, the concentration of the tetracycline antibiotic e.g., minocycline is about or at least about 0.05% by weight, is about or at least about 0.1% by weight, is about or at least about 0.5% by weight, is about or at least about 1% by weight, is about or at least about 1.5% by weight, is about or at least about 2% by weight, is about or at least about 2.5% by weight, is about or at least about 3% by weight, or is about or at least about 3.5% by weight, is about or at least about 4% by weight, is about or at least about 4.5% by weight, is about or at least about 5% by weight, is about or at least about 5.5% by weight, is about or at least about 6% by weight, is about or at least about 6.5% by weight or is about or at least about 7% by weight, is about or at least about 7.5% by weight, is about or at least about 8% by weight, is about or at least about 8.5% by weight, is about or at least about 9% by weight, is about or at least about 9.5% by weight, is about or at least about 10% by weight, or is about or at least about 10.5% by weight or is about or at least about 11% by weight. In one or more embodiments reference to by weight means by weight of the topical composition and with reference to foamable compositions without the addition of propellant.

In one or more embodiments, the therapeutic agent (e.g., a tetracycline antibiotic and/or a retinoid) is micronized.

In one or more embodiments, the therapeutic agent is not micronized.

In one or more embodiments, the active agent is micronized so that the diameter of 90% of the particles (d (0.9)), is less than about 30 microns, or less than about 20 microns, or less than about 10 microns. For example, less than about 28 microns, less than about 26 microns, less than about 24 microns, less than about 22 microns, less than about 20 microns, less than about 18 microns, less than about 16 microns, less than about 14 microns, less than about 12 microns, less than about 10 microns, less than about 8 microns, less than about 6 microns, less than about 4 microns, or less than about 2 microns. In some embodiments the average size of the micronized particles is about 30 microns to about 0.5 microns or about 25 microns to about 1 microns or about 20 microns to about 2 microns or about 15 microns to about 3 microns or about 12 microns to about 3.5 microns or about 10 microns to about 4 microns or about 9 microns to about 4.5 microns or about 8 microns to about 5 microns or about 7 microns to about 5.5 microns or a range between any two of the aforesaid amounts, such as about 12 microns to about 5 microns.

In one or more embodiments, the initial dose of the tetracycline antibiotic is about 18%, about 17.5%, about 16.5%, about 15.5%, about 14.5%, about 13.5%, about 12.5%, about 11.5%, about 10.5%, about 9.5%, about 8.5%, about 7.5%, about 6.5%, about 5.5%, about 4.5%, about 3.5%, about 2.5%, about 1.5%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.75%, about 0.5%, about 0.25%, or about 0.2% by weight of the composition. In one or more embodiments, the maintenance dose of the tetracycline antibiotic is about 7.5%, about 6.5%, about 5.5%, about 4.5%, about 3.5%, about 2.5%, about 1.5%, about 7%, about 6%, about 5%, about 4%, about 3%, about 2%, about 1%, about 0.5%, about 1.9%, about 1.8%, about 1.7%, about 1.6%, about 1.55, about 1.4%, about 1.3%, about 1.2%, about 1.1%, about 0.9%, about 0.8%, about 0.7%, about 0.6%, about 0.4%, about 0.35, about 0.25%, about 0.2%, about 0.15%, or about 0.1% by weight of the composition.

According to one or more embodiments, provided are foamable compositions comprising a tetracycline antibiotic, such as a minocycline, for use in treatment of acne conglobate, acne vulgaris or rosacea, and/or acne conglobate, acne vulgaris or rosacea related symptoms, and/or a tetracycline antibiotic responsive rosacea related disorder, and/or a tetracycline antibiotic responsive acne vulgaris related disorder, and/or a tetracycline antibiotic responsive skin disorder, and/or skin disorder caused by a bacteria, and/or a tetracycline antibiotic responsive disorder, and/or a sebaceous gland disorder. In one or more embodiments the tetracycline is used for the treatment of rosacea. In one or more embodiments the tetracycline antibiotic is used for the treatment of impetigo. In one or more embodiments the tetracycline antibiotic is used for the treatment of acne. In one or more embodiments the tetracycline antibiotic is used for the treatment of acne vulgaris. In one or more embodiments the tetracycline antibiotic is used for the treatment of non-inflammatory lesions. In one or more embodiments the tetracycline antibiotic is used for the treatment of inflammatory lesions. In one or more embodiments the tetracycline antibiotic is used for the treatment of comodones. In one or more embodiments the tetracycline antibiotic acts to reduce oxidative stress and/or inflammation in skin pathologies. In one or more embodiments the tetracycline antibiotic is effective where the condition is accompanied by apoptotic cell death.

In one or more embodiments, provided are foamable compositions comprising a minocycline or a doxycycline for use in treating acne conglobate, acne vulgaris or rosacea, and/or acne conglobate, acne vulgaris or rosacea related symptoms, and/or a tetracycline antibiotic responsive rosacea related disorder, and/or a tetracycline antibiotic responsive acne vulgaris related disorder, and/or a tetracycline antibiotic responsive skin disorder, and/or skin disorder caused by a bacteria, and/or a tetracycline antibiotic responsive disorder, and/or a sebaceous gland disorder. In one or more embodiments minocycline or doxycycline is used for the treatment of acne. In one or more embodiments minocycline or doxycycline is used for the treatment of acne conglobata or acne vulgaris. In one or more embodiments the tetracycline antibiotic is used for the treatment of inflammatory and/or non-inflammatory acne. In one or more embodiments the minocycline or doxycycline is used for the treatment of comodones. In one or more embodiments minocycline or doxycycline is used for the treatment of rosacea.

### Retinoid

Also contemplated are compositions, and/or foamable compositions comprising a wax, such as hydrogenated castor oil (e.g., about 0.1% to about 2%, or about 0.2% to about 1.4%, or about 0.3 to about 1.2% hydrogenated castor oil), a tetracycline antibiotic, and/or at least one additional active agent. Examples of active agents include but are not limited to retinoids, benzoyl peroxide, salicylic acid, alpha hydroxy acid, resorcinol, dicarboxylic acids e.g., azelaic acid, immunomodulators, e.g. JAK inhibitors (e.g., Toficitinib), and sulfur. Without being bound by theory, retinoids may be comedolytic, resolve the precursor microcomedone lesion, and provide anti-inflammatory effects. In some embodiments, an active agent in a composition, and/or foamable compositions disclosed herein comprises a retinoid. In some embodiments, the composition, and/or foamable compositions comprises about 0.1% to about 10% by weight of a retinoid, (e.g., about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.1% to about 0.5%, about 0.1% to about 0.4%, or about 0.1% to about 0.3% by weight of the composition. In some embodiments, the composition, and/or foamable composition, comprises about 0.1%, about 0.15%, about 0.2%, about 0.25%, about 0.3%, about 0.35%, about 0.4%, about 0.45%, or about 0.5% by weight of a retinoid (e.g., adapalene). In one or more embodiments, the concentration of the retinoid (e.g. adapalene). In some embodiments the concentration is about 0.01%, about 0.02%, about 0.03%, about 0.04%, about 0.05%, about 0.06%, about 0.08%, about 0.11%, about 0.13%, about 0.15%, about 0.17%, about 0.19%, about 0.275%, about 0.325%, about 0.375%, about 0.425%, about 0.475%, about 0.55%, about 0.6%, about 0.65%, or about 0.7% or can be a range between any two figures listed in this paragraph, such as, 0.05% to about 0.7%. In some embodiments, the retinoid comprises adapalene (also referred to as “ADP”). In one or more embodiments reference to by weight means by weight of the topical composition and with reference to foamable compositions without the addition of propellant.

### Encapsulation

In one or more embodiments, the active agent (e.g., tetracycline antibiotic) is encapsulated. In one or more embodiments, the active agent is encapsulated in particles, microparticles, nanoparticles, microcapsules, microspheres, nanocapsules, nanospheres, liposomes, niosomes, polymer matrices, silica-gels, graphite, nanocrystals, or microsponges. Without being bound by theory, such particles can have various functions, such as (1) protection of the drug from degradation; (2) modification of the drug release rate from the composition; (3) control of skin penetration profile; and (4) mitigation of adverse effects, due to the controlled release of the active agent from the encapsulation particles. Encapsulation is described in U.S. Publication No. 2015/0209296, which is incorporated by reference. In some embodiments, the tetracycline antibiotic is encapsulated. In one or more embodiments related to one or more of the foregoing, the tetracycline active ingredient is associated with solid, porous microcarriers, each having a hydrophobic surface. In one or more additional embodiments, the solid, porous microcarriers comprise a material selected from the group consisting of hydrophobic surface-modified silicon dioxide, porous polystyrene, porous polyamide, porous hydrophobic cellulose, and porous polytetrafluoroethylene. In one or more embodiments, the microcarrier possesses a porous structure for retaining the active ingredient, a hydrophobic surface, and is chemically non-reactive with the active ingredient. In one or more additional embodiments, the hydrophobic encapsulant comprises a material selected from the group consisting of mineral oil, petrolatum jelly, synthetic waxes, natural waxes, and silicone oils. In one or more embodiments, the average encapsulant particle size is less than 95 microns, less than 75 microns, less than 50 microns, less than 25 microns, less than 22 microns, or less than 15 microns. In some embodiments, the average encapsulated particle size of the tetracycline antibiotic is about 5.5 to about 10.5 microns, about 6 microns to about 10.5 microns, about 6.5 microns to about 10 microns, about 7 microns to about 9.5 microns, or about 7.5 microns to about 9 microns.

### Emollient

Also contemplated are compositions, and/or foamable compositions comprising one or more emollients. In some embodiments, the composition comprises a wax, such as hydrogenated castor oil (e.g., about 0.1% to about 2%, about 0.2% to about 1.4%, or about 0.3 to about 1.2% hydrogenated castor oil), and at least one or more emollients. Without being bound by theory, emollients may reduce scaling and itching, reduce inflammation, improve skin barrier function, and act as a carrier for active agents. In some embodiments the emollient comprises one or more vegetable oils. In some embodiments the emollient comprises one or more animal or fish oils. In some embodiments the emollient comprises one or more mineral oils. In some embodiments the emollient comprises combinations of two or more of vegetable, animal, fish and mineral oil. Examples of emollients include but are not limited to an avocado oil, isopropyl myristate, a mineral oil, a MCT oil, capric triglyceride, caprylic triglyceride, isopropyl palmitate, isopropyl isostearate, diisopropyl adipate, diisopropyl dimerate, a maleated soybean oil, octyl palmitate, cetyl lactate, cetyl ricinoleate, tocopheryl acetate, acetylated lanolin alcohols, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, an olive oil, a corn oil, a soybean oil, a canola oil, a cottonseed oil, a coconut oil, a sesame oil, a sunflower oil, a safflower oil, a borage seed oil, a syzigium aromaticum oil, a hempseed oil, a herring oil, a cod-liver oil, a salmon oil, a flaxseed oil, a wheat germ oil, an evening primrose oil, an essential oil, a silicone oil, dimethicone, cyclomethicone, polyalkyl siloxane, polyaryl siloxane, polyalkylaryl siloxane, a polyether siloxane copolymer, and poly(dimethylsiloxane)-(diphenyl-siloxane).

In some embodiments, the foamable composition comprises an emollient, wherein the emollient is coconut oil. In some embodiments, the foamable composition comprises an emollient, wherein the emollient is light mineral oil. In some embodiments, the foamable composition comprises an emollient, wherein the emollient is isopropyl myristate. In some embodiments, the foamable composition comprises an emollient, wherein the emollient is a coconut oil. In some embodiments, the foamable composition comprises an emollient, wherein the emollient is soybean oil. In some embodiments, the foamable composition comprises an emollient, wherein the emollient is cyclomethicone. In some embodiments, the foamable composition comprises at least one or more of an emollient, selected from cyclomethicone, coconut oil, light mineral oil, isopropyl myristate and soybean oil. In some embodiments an emollient is selected having similar or closely equivalent properties thereto.

In one or more embodiments the emollient is or comprises a hydrophobic oil.

In some embodiments, the composition, and/or a foamable composition comprises about 55% to about 95% of at least one emollient by weight. In some embodiments, the composition, and/or a foamable composition comprises about 58% to about 93%, about 60% to about 91%, about 62% to about 89%, about 64% to about 87%, about 66% to about 85%, about 68% to about 84%, about 70% to about 83%, about 72% to about 82% of at least one emollient by weight of the composition. In some embodiments, the composition and/or a foamable composition comprises about 40% to about 60% by weight of soybean oil. In some embodiments, the composition, and/or a foamable composition comprises about 45% to about 55% by weight of soybean oil. In some embodiments, the composition and/or a foamable composition comprises about 45%, about 46%, about 47%, about 48%, about 49%, about 50%, about 51%, about 52%, about 53%, about 54%, or about 55%, by weight of soybean oil. In some embodiments, the composition, and/or a foamable composition comprises about 20% to about 25% by weight of coconut oil. In some embodiments, the composition, and/or a foamable composition comprises about 22% to about 25%, or about 23% to about 24% by weight of coconut oil. In some embodiments, the composition, and/or a foamable composition comprises about 20%, about 21%, about 22%, about 23%, about 24%, or about 25% by weight of coconut oil. In some embodiments, the composition, and/or the foamable composition comprises about 23.6% by weight of coconut oil. In some embodiments, the composition, and/or the foamable composition comprises about 1% to about 8% by weight of mineral oil (e.g., light mineral oil). In some embodiments, the composition, and/or the foamable composition comprises about 2% to about 8%, about 3% to about 7.5%, about 3.5% to about 7%, or about 4% to about 7%, by weight of mineral oil. In some embodiments, the composition and/or the foamable composition comprises about 1%, about 1.2%, about 1.4%, about 1.6%, about 1.8%, about 2%, about 2.2%, about 2.4%, about 2.6%, about 2.8%, about 3%, about 3.2%, about 3.4%, about 3.6%, about 3.8%, about 4%, about 4.2%, about 4.4%, about 4.6%, about 4.8%, about 5%, about 5.2%, about 5.4%, about 5.6%, about 5.8%, about 6%, about 6.2%, about 6.4%, about 6.6%, or about 6.8% by weight of mineral oil. In some embodiments, the foamable composition, and/or the foamable composition comprises about 3.3% to about 6.6% by weight of mineral oil. In some embodiments, the composition, and/or the foamable composition comprises about 3% to about 7% by weight of cyclomethicone. In some embodiments, the composition, and/or the foamable composition comprises about 3.5% to about 6.5%, or about 3.8% to about 6.2%, or about 4% to about 6%, by weight of cyclomethicone. In some embodiments, the composition, and/or the foamable composition comprises about 3.4%, about 3.6%, about 3.8%, about 4%, about 4.2%, about 4.4%, about 4.6%, about 4.8%, about 5%, about 5.2%, about 5.4%, about 5.6%, about 5.8%, about 6%, about 6.2%, about 6.4%, about 6.6%, about 6.8%, or about 7% by weight of cyclomethicone.

### Foam Adjuvant

Also contemplated are compositions, and/or foamable compositions comprising one or more foam adjuvants. In some embodiments, a composition comprises a wax, such as hydrogenated castor oil (e.g., about 0.1% to about 2%, about 0.2% to about 1.4%, or about 0.3 to about 1.2% hydrogenated castor oil), and at least one or more foam adjuvants in combination with one or more active agents. It is postulated, without being bound by any theory, that the use of foam adjuvants contributes to stability, viscosity, and smoothness of the foamable composition, allowing active agents to be delivered efficaciously to a desired target. Foam adjuvants that may be used in compositions, and/or foamable compositions are known to persons with skill in the art, and include but are not limited to fatty acids, and fatty alcohols.

In some embodiments, a foam adjuvant can include a fatty alcohol. Long chain saturated and mono-unsaturated fatty alcohols, e.g., stearyl alcohol, erucyl alcohol, arachidyl alcohol and behenyl alcohol (docosanol) have been reported to possess antiviral, anti-infective, anti-proliferative and anti-inflammatory properties (see, U.S. Pat. No. 4,874,794). Longer chain fatty alcohols, e.g., tetracosanol, hexacosanol, heptacosanol, octacosanol, triacontanol, etc., are also known for their metabolism modifying properties, and tissue energizing properties.

In one or more embodiments, the fatty alcohol comprises or is selected from the group consisting of lauryl alcohol, myristyl alcohol, cetyl alcohol, stearyl alcohol, cetostearyl alcohol, arachidyl alcohol, behenyl alcohol, tetracosanol, hexacosanol, octacosanol, triacontanol, and tetratriacontanol or mixtures of any two or more thereof.

In some embodiments, a foam adjuvant can include a fatty acid. In one or more embodiments, the fatty acid comprises or is selected from the group consisting of dodecanoic acid, tetradecanoic acid, hexadecanoic acid, heptadecanoic acid, octadecanoic acid, eicosanoic acid, docosanoic acid, tetracosanoic acid, hexacosanoic acid, heptacosanoic acid, octacosanoic acid, triacontanoic acid, dotriacontanoic acid, tritriacontanoic acid, tetratriacontanoic acid, and pentatriacontanoic acid or mixtures of any two or more thereof.

In one or more embodiments, the carbon chain of the fatty alcohol or the fatty acid is substituted with a hydroxyl group.

In one or more embodiments, the composition is hydrophobic. In one or more embodiments, the composition comprises one or more hydrophobic oils and waxes. In one or more embodiments, the composition comprises one or more fatty alcohols. In one or more embodiments, the composition comprises one or more waxes. In one or more embodiments, the composition comprises one or more fatty acids.

In one or more embodiments, the foam adjuvant is about 0.1% to about 20% by weight of the composition, and/or foamable compositions. In some embodiments, the compositions, and/or foamable compositions comprise about 0.2% to about 15%, about 0.3% to about 14%, about 0.4% to about 13%, about 0.5% to about 12% of a foam adjuvant (e.g., stearyl alcohol, myristyl alcohol, cetyl alcohol, cetostearyl alcohol, and/or behenyl alcohol). In some embodiments the compositions and/or foamable compositions comprise about 0.6% to about 11%, about 0.7% to about 10%, or about 0.8% to about 9%, of a foam adjuvant. In some embodiments the compositions and/or foamable compositions comprise about 0.8% to about 8%, about 0.8% to about 7%, about 0.7% to about 7%, about 0.5% to about 7%, about 0.6% to about 6%, about 0.7% to about 5%, about 0.8% to about 3%, about 1% to about 2%, or about 1.2% to about 1.8% by weight of a foam adjuvant. In some embodiments, the compositions, and/or foamable compositions comprise about 0.1%, about 0.25%, about 0.5%, about 0.75%, about 1%, about 1.1%, about 1.2%, about 1.4%, about 1.5%, about 1.6%, about 1.8%, about 2%, about 2.2%, about 2.4%, about 2.5%, about 2.6%, about 2.8%, about 3%, about 3.2%, about 3.4%, about 3.5%, about 3.6%, about 3.8%, about 4%, about 4.2%, about 4.4%, about 4.6%, about 4.8%, about 5%, about 5.5% by weight, about 6%, about 6.5%, about 7%, about 7.5%, about 8%, or about 8.5% by weight individually of a foam adjuvant).

In one or more embodiments, the foam adjuvant is stearic acid. In one or more embodiments, the foam adjuvant is docosanol. In one or more embodiments, the foam adjuvant is stearyl alcohol. In some embodiments, the compositions, and/or foamable compositions comprise about 1% to about 2% by weight of stearyl alcohol. In some embodiments, the compositions, and/or foamable compositions comprise about 1.2% to about 1.8% by weight of stearyl alcohol. In some embodiments, the compositions and/or foamable compositions comprise about 1.5% by weight of stearyl alcohol. In one or more embodiments, the foam adjuvant is cetostearyl alcohol. In some embodiments, the compositions and/or foamable compositions comprise about 2% to about 5% by weight of cetostearyl alcohol. In some embodiments, the compositions and/or foamable compositions comprise about 3% to about 4% by weight of cetostearyl alcohol. In some embodiments, the compositions and/or foamable compositions comprise about 3.5% by weight of cetostearyl alcohol. In one or more embodiments, the foam adjuvant is myristyl alcohol. In some embodiments, the compositions and/or foamable compositions comprise about 1.8% to about 3.3% by weight of myristyl alcohol. In some embodiments, the compositions, and/or foamable compositions comprise about 2% to about 3% by weight of myristyl alcohol. In some embodiments, the compositions and/or foamable compositions comprise about 2.5% by weight of myristyl alcohol.

### Wax

In one or more embodiments, the compositions, and/or foamable compositions comprise at least one or more of a wax. In some embodiments the wax can assist in foaming. Without being bound by theory, waxes are lipophilic and soluble in hydrophobic solvents, and may provide a suitable carrier for active agents that are otherwise easily degraded when exposed to water or other pharmaceutical excipients. When the compositions described herein, and/or foamable compositions are prepared by the methods described herein, waxes may aid the compositions, and/or foamable compositions in forming crystals with nonuniform shapes, plates, or spherulites and may contribute substantially to the crystal fingerprint of such compositions. Waxes, or mixtures of waxes may also control the viscosity of the foamable compositions, allowing them to flow or be shakable.

In some embodiments, a wax used in a composition disclosed herein has a melting temperature of about 36° C. or higher. In some embodiments, a wax has a melting temperature of about 49° C. or higher, or about 81° C. or higher. In one or more embodiments the wax is solid at 36° C. or more. In one or more embodiments the wax is solid at 49° C. or more. In one or more embodiments the wax is solid at 81° C. or more. In one or more embodiments, the formulations provided herein comprise a wax, wherein within the formulation said wax has a melting point of 68-69° C. In one or more embodiments, the formulations provided herein comprise a wax, wherein within the formulation said wax has a melting point of 42-44° C. In some embodiments, foamable compositions comprise at least one or more of a wax selected from the group consisting of a plant wax, a white wax, an emulsifying wax, a caranuba wax, a candelilla wax, a cerasine/ozokerite wax, a Japan wax, a castor wax, a microcrystalline wax, a montan wax, a peat wax, an ouricury wax, a sugarcane wax, a retamo wax, a jojoba oil, an animal wax (e.g. lanolin, spermaceti wax, and wool fat), beeswax, a shellac wax, a hydrogenated oil (e.g., a hydrogenated castor oil, or a hydrogenated cotton seed oil), a petroleum derived wax, a paraffin wax, polyethylene wax, and derivatives thereof.

Waxes that remain solid at room or body temperature may be used in the formulations disclosed herein and may form microcrystals that are homogenously distributed within the foamable formulations described herein. Without being bound by theory, the type, number, and distribution of crystals within the formulation may change its rheological properties, ultimately having an effect on the stability and usability of the formulation. In some embodiments, larger wax crystals may also be found distributed within the formulation.

In one or more embodiments, compositions and/or foamable compositions comprising a wax, such as hydrogenated castor oil (e.g., about 1.2% hydrogenated castor oil) further comprise a combination of two waxes, or sometimes three waxes, or sometimes four waxes, or sometimes five waxes, or six or more waxes.

In one or more embodiments, compositions, and/or foamable compositions comprise about 0.1% to about 7% by weight of a wax. In one or more embodiments, compositions, and/or foamable compositions comprise about 0.1% to about 6%, about 0.1% to about 5%, about 0.1% to about 4% about 0.15% to about 3.7%, 0.2% to about 3.6%, about 0.3% to about 3.5%, about 0.4% to about 3.4%, about 0.5% to about 3.3%, about 0.6% to about 3.2%, about 0.7% to about 3.1%, about 0.8% to about 3%, about 1% to about 3%, about 0.8% to about 2.75%, 0.8% to about 2.5%, about 0.8% to about 2.25%, about 0.8% to about 2%, about 1% to about 2%, about 1.1% to about 1.9%, or about 1.2% to about 1.8% by weight of a wax. In one or more embodiments, compositions, and/or foamable compositions comprise about 0.1%, about 0.25%, about 0.5%, about 0.75%, about 1%, about 1.2%, about 1.4%, about 1.5%, about 1.6%, about 1.8%, about 2%, about 2.2%, about 2.4%, about 2.6%, about 2.8%, about 3%, about 3.2%, about 3.4%, about 3.5%, about 3.6%, about 3.8%, about 4%, about 4.2%, about 4.4%, about 4.6%, about 4.8%, about 5%, about 5.5% or about 6% by weight individually of each wax (e.g. white wax) or by total weight of all waxes (e.g., a hydrogenated castor oil (HCO), and beeswax (white wax). In one or more embodiments, a foamable composition comprises about 0.1% to about 3% by weight of a HCO. In one or more embodiments, foamable compositions comprise about 0.4% to about 2.2% by weight of HCO. In one or more embodiments, foamable compositions comprise about 0.6% to about 1.8%, or about 0.8% to about 1.6% by weight of HCO. In one or more embodiments, foamable compositions comprise about 1% to about 1.4% by weight of HCO. In one or more embodiments, foamable compositions comprise about 1.2% by weight of HCO. In one or more embodiments, foamable compositions comprise about 0.1% to about 4% by weight of a white wax. In one or more embodiments, foamable compositions comprise about 1% to about 3% by weight of a white wax. In one or more embodiments, foamable compositions comprise about 2% by weight of a white wax.

In one or more embodiments, the ratio of HCO to beeswax in the formulation is between about 1:20 and about 20:1, for example about 1:20, about 1:19, about 1:18, about 1:17, about 1:16, about 1:15, about 1:14, about 1:13, about 1:12, about 1:11, about 1:10, about 1-9, about 1:8, about 1:7, about 1:6, about 1:5, about 1:4, about 1:3, about 1:2, about 1:1, about 2:1, about 3:1, about 4:1, about 5:1, about 6:1, about 7.1, about 8:1, about 9:1, about 10:1, about 11:1, about 12:1, about 13:1, about 14:1, about 15:1, about 16:1, about 17:1, about 18:1, about 19:1, or about 20:1. In some embodiments, the ratio of HCO to beeswax in the formulation is between about 1:12 and about 12:1, for example between about 1:10 and about 10:1, about 1:8 and about 8:1, about 1:6 and about 6:1 about 1:4 and about 4:1, about 3:10 and about 10:3, about 1:2 and about 2:1 about 3:5 and about 5:3, or about 2:3 and about 3:2. In some embodiments the ratio is about 1:1. In one or more embodiments, the ratio between HCO and beeswax in the formulation is between about 0.3:1 and about 1.7:1. For example about 0.4:1, or about 0.5:1, or about 0.6:1, or about 0.7:1, or about 0.8:1, or about 0.9:1, or about 1:1, or about 1.2:1, or about 1.3:1, or about 1.4:1, or about 1.5:1, or about 1.6:1, or about 1.7:1.

### Silica

In one or more embodiments, the composition comprises about 0.1% w/w to about 0.4% w/w of fumed (modified) silica or silica dioxide. In one or more embodiments, the composition comprises about 0.125% w/w to about 0.35%, or about 0.15% to about 3% w/w of fumed (modified) silica or silica dioxide. In some embodiments, the composition is substantially free of fumed (modified) silica or silica dioxide. In some embodiments, the composition is essentially free of fumed (modified) silica or silica dioxide. In some embodiments, the composition is free of fumed (modified) silica or silica dioxide.

### Propellant

In some embodiments, the foamable compositions described herein are packaged in an aerosol container and pressurized with a propellant. In one or more embodiments, the foamable composition further comprises a propellant. Any compatible propellant can be used. In one or more embodiments, the propellant is a gas at room temperature under normal pressure and which can be liquefied at increased pressure at room temperature. Examples of propellants include, without limitation, hydrocarbon propellants such as butane, propane, isobutane, dimethyl ether, fluorocarbons such as 1,1,1,2 tetrafluoroethane (Dymel 134a), and 1,1,1,2,3,3,3 heptafluoropropane (Dymel 227), and mixtures thereof. In one or more embodiments, the foamable compositions described herein are pressurized with a hydrocarbon mixture comprising butane, propane, and isobutene. In some embodiments, the foamable compositions described herein are pressurized with a hydrocarbon mixture comprising AP-70 (a mixture of about 30% w/w butane, 20% w/w isobutane and 50% w/w propane) is used. In some embodiments, the foamable compositions described herein are pressurized with a hydrocarbon mixture comprising AP-46 (about 16% w/w of propane, about 82% w/w of isobutane and about 2% w/w of propane). In some embodiments, the foamable compositions described herein are pressurized with hydro fluorocarbon (HFC) propellants. In one or more embodiments, the foamable compositions described herein are pressurized with compressed gases (e.g., air, carbon dioxide, nitrous oxide, and nitrogen).

In some embodiments, the propellant is a self-foaming propellant, i.e., a volatile liquid having a boiling point of less than the temperature of the target treatment site (such as the skin). An example of a post-foaming propellant is isopentane (bp=26° C.). In some embodiments, the propellant is isopentane.

Any concentration of the propellant, which forms an acceptable foam, is useful in accordance with the present invention. In some embodiments the propellant makes up between about 1% to about 30% of the foamable composition, or about 3% to 30%, about 3% to 25%, about 4% to about 20%, or about 5% to about 18% of the composition. In preparing the formulations the ingredients other than propellant are combined to 100% and the propellant is added thereafter so that the ratio of formulation to propellant can range from about 100:1 to about 100:30, about 100:3 to about 100:30, about 100:3 to about 100:25, about 100:4 to 100:20, or about 100:5 to about 100:18. In some embodiments, the ratio of formulation to propellant is between about 100:20 and about 100:50.

Due to environmental concerns, as well as compatibility considerations, propellants that are not environmentally friendly are to be avoided. In some embodiments, the formulations do not comprise chlorofluorocarbons (CFCs).

It may be possible to reduce the amount of foam adjuvants in the formulation but still provide good expulsion from the canister by mixing some of the propellant in the formulation and keeping some of it separate. By way of example, the propellant could be mixed with the formulation at between about 1% to 3%, between about 2% to 4%, or between about 3% to 5% propellant (ratio of formulation to propellant of 100:1 to 100:3, 100:2 to 100:4, or 100:3 to 100:5 respectively) while keeping a further amount of propellant separate from the formulation. In one or more embodiments, the propellant can also be used to expel the formulation using a bag-in-can system or a can-in-can system as will be appreciated by someone skilled in the art. In some embodiments part of the propellant system is in the formulation and part of it is kept separate from the formulation using a bag-in-can or can-in-can system. In one or more embodiments a pump or other mechanical means is used to provide expulsion force.

### Other Ingredients

In certain embodiments, the composition is free of one or more of a petrolatum, surface active agents, protic solvents, polar aprotic solvents, isopropyl myristate, polyethylene gelling agents, polyethylene homopolymers, polyethylene copolymers, selenium derivatives and silicone thickening agents. In some embodiments, the foamable composition is essentially free of one or more of petrolatum, surface active agents, protic solvents, certain polar aprotic solvents, isopropyl myristate, polyethylene gelling agents, polyethylene homopolymers, polyethylene copolymers, selenium derivatives and silicone thickening agents. In some embodiments, the foamable composition is substantially free of one or more of petrolatum, surface active agents, protic solvents, certain polar aprotic solvents, isopropyl myristate, polyethylene gelling agents, polyethylene homopolymers, polyethylene copolymers, selenium derivatives and silicone thickening agents. In some embodiments, the composition comprises less than about 0.4% by weight of each one or more of petrolatum, surface active agents, protic solvents, certain polar aprotic solvents, isopropyl myristate, polyethylene gelling agents, polyethylene homopolymers, polyethylene copolymers, selenium derivatives and silicone thickening agents. In some embodiments, the composition comprises less than about 0.4% by weight in total of one or more of petrolatum, surface active agents, protic solvents, certain polar aprotic solvents, isopropyl myristate, polyethylene gelling agents, polyethylene homopolymers, polyethylene copolymers, selenium derivatives and silicone thickening agents. In some embodiments, the composition comprises less than about 0.2% by weight of each one or more of petrolatum, surface active agents, protic solvents, certain polar aprotic solvents, isopropyl myristate, polyethylene gelling agents, polyethylene homopolymers, polyethylene copolymers, selenium derivatives and silicone thickening agents. In some embodiments, the composition comprises less than about 0.2% by weight in total of one or more of petrolatum, surface active agents, protic solvents, polar aprotic solvents, isopropyl myristate, polyethylene gelling agents, polyethylene homopolymers, polyethylene copolymers, selenium derivatives and silicone thickening agents. In some embodiments, the composition comprises less than about 0.1% by weight of each one or more of petrolatum, surface active agents, protic solvents, certain polar aprotic solvents, isopropyl myristate, polyethylene gelling agents, polyethylene homopolymers, polyethylene copolymers, selenium derivatives and silicone thickening agents. In some embodiments, the composition comprises less than about 0.1% by weight in total of one or more of petrolatum, surface active agents, protic solvents, certain polar aprotic solvents, isopropyl myristate, polyethylene gelling agents, polyethylene homopolymers, polyethylene copolymers, selenium derivatives and silicone thickening agents.

In one or more embodiments, the composition, and/or foamble composition is substantially alcohol-free, i.e., substantially free of short chain alcohols having up to 5 carbon atoms in their carbon chain skeleton. In other embodiments, the composition comprises less than about 5% by weight final concentration of short chain alcohols, for example, less than 2% by weight, or less than 1% by weight. In certain embodiments, the composition is free, or essentially free of ethanol, propanol, butanol and pentanol.

### Surface Active Agents

In some embodiments, the composition and/or foamable composition disclosed herein further comprises a surfactant. In some embodiments the surfactant is non-ionic. In some embodiments the surfactant is ionic. In some embodiments the surfactant is zwitterionic. In some embodiments, the foamable composition comprises less than about 10% of a surfactant, e.g., less than about 5%, less than about 3%, less than about 2%, less than about 1%, or less than about 0.1% of a surfactant. In some embodiments, the formulations disclosed herein are substantially surfactant-free. In some embodiments, the formulations disclosed herein are essentially surfactant-free. In some embodiments, the formulations disclosed herein are surfactant-free.

In some embodiments, a formulation disclosed herein lacks a non-ionic surfactant. Non-limiting examples of excluded classes of non-ionic surfactants include: (i) polyoxyethylene sorbitan esters (polysorbates), such as polysorbate 20, polysorbate 40, polysorbate 60 and polysorbate 80; (ii) sorbitan esters, such as sorbitan monolaurate and sorbitan monooleate; (iii) polyoxyethylene fatty acid esters, such as, PEG-8 stearate, PEG-20 stearate, PEG-40 stearate, PEG-100 stearate, PEG-150 distearate, PEG-8 laurate, PEG-10 laurate, PEG-12 laurate, PEG-20 laurate, PEG-8 oleate, PEG-9 oleate, PEG-10 oleate, PEG-12 oleate, PEG-15 oleate and PEG-20 oleate; (iv) PEG-fatty acid diesters; (v) polyethylene glycol (PEG) ethers of fatty alcohols; (vi) glycerol esters, such as glyceryl monostearate, glyceryl monolaurate, glyceryl monopalmitate and glyceryl monooleate; (vii) PEG-fatty acid mono- and di-ester mixtures; (viii) polyethylene glycol glycerol fatty acid esters; (ix) propylene glycol fatty acid esters; (x) mono- and diglycerides; (xi) sugar esters (mono-, di- and tri-esters of sucrose with fatty acids) and (xii) PEG alkyl phenols.

In certain embodiments, the composition can comprise of one or more of a petrolatum, surface active agents, protic solvents, polar aprotic solvents, isopropyl myristate, polyethylene gelling agents, polyethylene homopolymers, polyethylene copolymers, selenium derivatives and silicone thickening agents.

In some embodiments the composition is free or essentially free of an active agent or an unstable active agent and comprises of one or more of a petrolatum, surface active agents, protic solvents, polar aprotic solvents, isopropyl myristate, polyethylene gelling agents, polyethylene homopolymers, polyethylene copolymers, selenium derivatives and silicone thickening agents.

In some embodiments, the composition comprises an unstable active agent and can also comprise of one or more of a petrolatum, surface active agents, protic solvents, polar aprotic solvents, isopropyl myristate, polyethylene gelling agents, polyethylene homopolymers, polyethylene copolymers, selenium derivatives and silicone thickening agents. To the extent the active agent is or may not be compatible with one or more of the aforesaid the unstable agent may be protected by one or more of encapsulation, complexing with other agents such as metal agents or salts, inclusion of anti-oxidants, and other similar methods known to one skilled in the art.

### Protic Solvents

In certain embodiments, the compositions disclosed herein comprise protic solvents, such as short chain alcohols, glycols and glycerin. In some embodiments, the compositions disclosed herein are free, or essentially free, or substantially free of protic solvents.

### Aprotic Polar Solvents

In certain embodiments, the compositions disclosed herein comprise aprotic solvents. In some embodiments, the compositions disclosed herein are free, or essentially free, or substantially free of aprotic solvents.

PCT Publication No. WO11/039637 indicates that certain polar aprotic solvents may be incompatible with tetracycline antibiotics. In some embodiments, formulations disclosed herein are free, or essentially free, or substantially free of aprotic polar solvents, such as dimethyl sulfoxide (DMSO), dimethylformamide (DMF), acetonitrile, acetone, methyl ethyl ketone, 1,4-Dioxane and tetrahydrofuran (THF), N-methylpyrrolidone, pyridine, piperidine, dimethylformamide, N-methyl-2-pyrrolidone and 1-methyl-2-pyrrolidinone) and azone (1-dodecylazacycloheptan-2-one).

### Silicone Thickening Agents

In some embodiments, compositions disclosed herein may comprise one or more silicone thickening agents, such as an elastomer. In some embodiments, a thickening agent is used in place of soybean oil, e.g., cyclopentasiloxane and dimethicone are used. Silicone thickening agents may comprise one or more polysiloxane-derived components. Such polysiloxanes are typically cross-linked, have rubber-like characteristics, and require solubilization in an oil, e.g., a silicone oil. An example of a silicone thickening agent is the ST-Elastomer 10 (Dow Corning), which is a mixture of high molecular weight dimethicone crosspolymer (12%) in cyclopentasiloxane (cyclomethicone, silicone solvent). Further, in the context of forming a breakable foam, cyclomethicone is known as a defoamer and therefore its presence in high concentrations in the foamable composition is undesirable. In one or more embodiments the compositions and/or foamable compositions comprise one or more silicone thickening agents. In some embodiments the composition and/or the foamable composition is substantially free of a silicone thickening agent. In some embodiments the composition and/or the foamable composition is essentially free of a silicone thickening agent. In some embodiments the composition and/or the foamable composition is free of a silicone thickening agent. In some embodiments, the composition is free or substantially free of silicone thickening agents other than cyclomethicone.

In some embodiments, elastomers are provided, e.g., in high amounts, e.g., in the range of about 86% to about 96% by weight of the composition, for example, about or more than 86%, 88%, 90%, 92%, 94%, 95%, or 96%. In some embodiments, elastomers are provided in amounts in the range of about 30% to about 54% by weight of the composition, for example, about or more than 34%, 38%, 42%, 46%, 50% or 54%. In some embodiments, the compositions and/or foamable compositions is substantially free of elastomers. In one or more other specific embodiments, the compositions and/or foamable compositions is essentially free of elastomers. In some embodiments, the compositions and/or foamable compositions comprises less than about 30% silicones, less than about 25% silicones, less than about 20% silicones, less than about 15% silicones, less than about 10% silicones, less than about 7.5% silicones, less than about 5% silicones, less than about 2% silicones, less than about 1% silicones, less than about 0.75% silicones, less than about 0.5% silicones, or less than about 0.25% silicones. In some embodiments, the compositions and/or foamable compositions comprises about 1% to about 5% silicones, or about 0.5% to about 3% silicones. In some embodiments, the compositions and/or foamable compositions does not comprise a silicone other than cyclomethicone. In some embodiments, In some embodiments, the compositions and/or foamable compositions does not comprise one or more volatile silicones. In some embodiments, volatile silicones are present at about 3% or less.

In one or more embodiments, semi-solid hydrophobic oils are a subsidiary component in the composition, for example being present at less than about 45%, at less than about 40%, at less than about 35%, at less than about 30%, at less than about 25%, less than about 20%, less than about 15%, less than about 10%, or less than about 5% by weight of the composition. In one or more alternative embodiments, semi-solid oils are omitted.

### Polyols

In some embodiments the compositions and/or foamable compositions may comprise a polyol. The identification of a “polyol,” as used herein, is an organic substance that contains at least two hydroxyl groups in its molecular structure. In one or more embodiments, the polyol is a diol (a compound that contains two hydroxyl groups in its molecular structure e.g. propylene glycol).

In one or more embodiments, the polyol is a triol (a compound that contains three hydroxyl groups in its molecular structure, such as glycerin).

In one or more embodiments, the polyol is a saccharide. Exemplary saccharides include, but are not limited to, monosaccharides (e.g., ribose, glucose, fructose, and galactose), disaccharides (such as sucrose, maltose, and/or lactose), oligosaccharides, and sugar alcohols (e.g. mannitol, sorbitol, xylitol, maltitol, lactitol).

Mixtures of polyols, including (1) at least one polyol comprises or selected from a diol and a triol; and (2) a saccharide are contemplated within the scope of the present disclosure.

According to some embodiments, the composition is polyol free, i.e., free of polyols. In some embodiments the composition and/or the foamable composition is essentially free of polyols. In some embodiments, the composition and/or foamable composition is substantially free or essentially free or free of a diol. In some embodiments, the composition and/or foamable composition is substantially free or essentially free or free of a triol. In some embodiments, the composition and/or foamable composition is substantially free or essentially free or free of a saccharide. In some embodiments, the composition and/or foamable composition is substantially free or essentially free or free of a sugar alcohol.

In some embodiments, the composition and/or foamable composition comprises less than about 5%, less than about 2%, less than about 1%, or less than about 0.5% by weight of polyols. In some embodiments, the composition and/or foamable composition comprises about 1% to about 5%, or about 0.5% to about 3% by weight of polyols.

In one or more embodiment, a composition can include one or more additional components. Such additional components include but are not limited to anti-static agents, buffering agents, bulking agents, chelating agents, cleansers, colorants, conditioners, deodorants, diluents, dyes, emollients, fragrances, humectants, perfuming agents, permeation enhancers, pH-adjusting agents, preservatives, protectants, skin penetration enhancers, softeners, solubilizers, sunscreens, sun blocking agents, viscosity modifiers and vitamins. As is known to one skilled in the art, in some instances a specific additional component may have more than one activity, function or effect.

In one or more embodiments there is provided a composition and/or a foamable composition comprising: an unstable active agent, such as a tetracycline antibiotic alone or in combination with one or more other active ingredients such as a retinoid; and a wax, such as a hydrogenated castor oil, wherein the wax is present in the composition in an amount effective to form a stable foamable formulation. In some embodiments the wax comprises a hydrogenated oil. In some embodiments the wax comprises hydrogenated castor oil. In some embodiments the tetracycline antibiotic is tetracycline, oxytetracycline, demeclocycline, doxycycline hyclate, lymecycline, meclocycline, methacycline, minocycline hydrochloride, rolitetracycline, chlorotetracycline, or tigecycline. In some embodiments the tetracycline antibiotic is present in the composition at a concentration of about 0.5% to about 10% by weight; about 1% to about 4% by weight; about 1.5% by weight; or about 3% by weight. In some embodiments the tetracycline antibiotic is a minocycline. In some embodiments the minocycline is minocycline hydrochloride. In some embodiments the minocycline hydrochloride is present in the composition at a concentration of about 1.5% by weight; about 3.0% by weight, or between about 1.5% and about 3% by weight. In one or more embodiments there is provided a composition and/or a foamable composition comprising a tetracycline antibiotic and an additional active agent. In some embodiments, the additional active agent comprises a retinoid. In some embodiments the retinoid is adapalene or tazarotene. In some embodiments the retinoid is adapalene. In some embodiments the retinoid is present in the composition at a concentration of about 0.1% to about 1% by weight, about 0.1% to about 0.5% by weight; about 0.2% by weight, about 0.3% by weight, or about 0.4% by weight. In one or more embodiments the retinoid is adapalene and the adapalene is present in the composition at a concentration of about 0.1% to about 0.5% by weight, or between about 0.1% to about 0.3% by weight. In some embodiments the composition and/or foamable composition comprises about 1-3%, or about 1-2%, or about 1.2%, hydrogenated castor oil. In some embodiments the composition and/or foamable composition comprises about 1.2% hydrogenated castor oil.

In one or more embodiments the composition and/or foamable composition further comprises (a) about 60% to about 95% by weight of at least one emollient and (b) about 5% to about 25% by weight at least one foam adjuvant, or a combination thereof. In one or more embodiments the composition comprises: a) about 40% to about 60% by weight of soybean oil; b) about 20% to about 25% by weight of coconut oil; c) about 2% to about 8% by weight of light mineral oil; d) about 2% to about 4% by weight of stearic acid; e) about 0.6% to about 1.6% by weight of docosanol; f) about 1% to about 2% by weight of hydrogenated castor oil; g) about 1% to about 3% by weight of white wax (such as beeswax); h) about 1% to about 2% by weight of stearyl alcohol; i) about 2.0% to about 5.0% by weight of cetostearyl alcohol; j) about 1.8% to about 3.3% by weight of myristyl alcohol; k) about 3.0% to about 7.0% by weight of cyclomethicone; I) about 1% to about 5% (such as 1%, 1.5%, 2%, 2.5%, 3%, 3.5%, 4%, 4.5%, or 5%) by weight of minocycline hydrochloride and m) about 0.1% to about 0.5% (such as 0.1%, 0.2%, 0.3%, 0.4%, or 0.5%) by weight of adapalene.

In some embodiments the composition and/or the foamable composition comprises: a) about 40% to about 60% by weight of soybean oil; b) about 20% to about 25% by weight of coconut oil; c) about 2% to about 8% by weight of light mineral oil; d) about 2% to about 4% by weight of stearic acid; e) about 0.6% to about 1.6% by weight of docosanol; f) about 0.1% to about 2% by weight of hydrogenated castor oil; g) about 0.1% to about 3% by weight of a white wax (such as beeswax); h) about 1% to about 2% by weight of stearyl alcohol; i) about 2.0% to about 5.0% by weight of cetostearyl alcohol; j) about 1.8% to about 3.3% by weight of myristyl alcohol; k) about 3.0% to about 7.0% by weight of cyclomethicone; I) about 1% to about 5% (such as 1%, 1.5%, 2%, 2.5%, 3%, 3.5%, 4%, 4.5%, or 5%) by weight of minocycline hydrochloride and m) about 0.1% to about 0.5% (such as 0.1%, 0.2%, 0.3%, 0.4%, or 0.5%) by weight of adapalene.

In some embodiments the composition and/or foamable composition comprises:
i) about 50% by weight of soybean oil;
ii) about 23.6% by weight of coconut oil;
iii) about 3.3% to about 6.6% by weight of light mineral oil;
iv) about 3% by weight of stearic acid;
v) about 1.1% by weight of docosanol;
vi) about 1.2% by weight of hydrogenated castor oil;
vii) about 3.5% by weight of cetostearyl alcohol;
viii) about 2% by weight of white wax (such as beeswax);
ix) about 1.5% by weight of stearyl alcohol;
x) about 2.5% by weight of myristyl alcohol;
xi) about 5% by weight of cyclomethicone;
xii) about 3% by weight of minocycline hydrochloride; and
xiii) about 0.3% by weight of adapalene.

In one or more embodiments the above composition and/or foamable composition is characterized e.g. by comprising Tmh crystals.

In one or more embodiments the compositions and/or foamable compositions comprising novel Tmh crystals are characterized by one or more of the following markers as illustrated in the examples: a higher final melting temperature; a higher enthalpy, e.g., as measured by DSC (calculated, e.g., as the area by integration under the endothermic curve); a higher intensity of x ray diffraction when measured by small angle x-ray crystallography; a higher number or intensity of Van Der Waals forces e.g., as measured by Infrared Spectroscopy; more hydrogen bonds e.g., as measured by Infrared Spectroscopy; a change in the number of hydrophobic interactions, which may lead to a more stable and/or more fluid or flowable) formulation; a raman spectra in the range of about 1400-1500 cm⁻¹ with a peak at about 1446 cm⁻¹ having one or two shoulders at about 1465 cm⁻¹ and/or at about 1425 cm⁻¹; and/or a band of lower frequency and or of higher intensity as measured by FTIR and/or lower flow point temperature e.g. the temperature which G″ becomes higher than G′. Without being bound by theory, a change in small angle x-ray crystallography may be observed between Tmh crystals and spherulites or plates, e.g., crystals obtained when using a hold step, due to the fact that the Tmh crystals are more closely packed, which may correlate with a higher Tm4.

In one or more embodiments, the composition and/or foamable composition of the present disclosure contains a fragrance. In one or more embodiments, the fragrance is at a concentration of about 0.1% by weight to about 1%, or about 0.2% by weight to about 0.8% by weight.

In one or more embodiments, the composition is applied as a gel, paste, lotion, spray, mask, patch, pomade, ointment, oil, foam or mousse.

### Methods of Preparing Foamable Compositions

The present disclosure also provides methods of preparing foamable compositions. In some embodiments, the method comprises a continuous heating-cooling process. In some embodiments, the method comprises a holding process. In one or more embodiments the formulation is mixed during each process. In one or more embodiments mixing may optionally be suspended for addition of ingredients.

Provided herein are methods of preparing a foamable composition comprising a wax, such as hydrogenated castor oil (such as about 1-3% hydrogenated castor oil, e.g., about 1.2%). In some embodiments, the foamable composition further comprises at least one emollient and/or at least one foam adjuvant. In some embodiments, the foamable composition further comprises one or more active agents, e.g., a tetracycline antibiotic (such as a minocycline e.g., MCH) and/or a retinoid (such as adapalene). In some embodiments, the formulation does not comprise an active agent.

In some embodiments, the method of preparing a foamable composition does not comprise a holding process as described herein and instead the melt is cooled in a continuous process. In some embodiments sensitive components are added during the cooling process whilst mixing to homogeneity within certain temperature ranges such as about 35° C. to about 40° C. or about 24° C. to about 28° C.. In one or more embodiments mixing to homogeneity is for up to an hour or about an hour. In one or more embodiments mixing to homogeneity is more than an hour (e.g., about 1-2 hours). In one or more embodiments cooling to a lower temperature continues after the formulation is homogenous. In some embodiments, the method of preparing a foamable composition comprises a holding process. In some embodiments, the holding process comprises (a) providing a wax, such as hydrogenated castor oil (e.g., about 1.2%) and mixing it with any other excipients (such as at least one emollient and/or at least one foam adjuvant), and heating the mixture to a temperature sufficient to completely melt the mixture or to a temperature at which a homogenous mixture can be observed; (b) cooling the mixture to a temperature of about 54° C., and holding at that temperature for about 1-10 hours, e.g., about 1-8 hours, or about 1-5 hours, or about 1-4 hours, or about 1-3 hours; e.g., or about 8 hours, or about 6 hours, or about 5 hours, or about 4 hours, or about 3 hours, or about 2 hours; (c) cooling the mixture to a temperature of about 35° C. to about 40° C.; (d) optionally adding an active agent such as a tetracycline antibiotic and/or an emollient, such as cyclomethicone, to the mixture; (e) cooling the mixture to a temperature of about 24° C. to about 28° C.; (f) optionally adding an additional active agent, such as a retinoid to the mixture; (g) cooling the mixture to room temperature (e.g., a temperature of about 22° C. to about 28° C.); and (h) stirring the mixture for up to 24 hours at room temperature (e.g., a temperature of about 20° C. to about 24° C.).

In some embodiments, one or more process modifications are contemplated which may affect shakability. In some embodiments, a holding process comprising holding at about 52° C. for 4 hours and then heating back to about 65° C. results in formulations with improved shakability as compared to those prepared a continuous heating-cooling process, but less shakable than those prepared at holding at about 54° C. for 4 hours (compare Table 17 Example 7, compared to Table 11A in Example 3). In some embodiments, a continuous process wherein the 1.2% hydrogenated castor is added at 22° C., followed by a continuous heating-cooling procedure results in moderately-shakable or non-shakable formulations. In some embodiments, both formulations show DSC thermograms with faded T_{M4} and are different from those measured for formulations prepared by a holding process at about 54° C. 4 hr. It may be, without being bound by theory, that warming to a higher temperature after a holding period may reduce or eliminate the Tmh crystals which in turn reduces shakability.

In some embodiments, a formulation prepared by holding for 30 min at 55-58° C. is moderately shakable on Day 30 at 25° C..

In some embodiments the holding temperature and the holding period may impact the prevention of reduction in Tm4 when adding a wax e.g. hydrogenated castor oil (compare, e.g., the drop in Tm4 reported in Example 8 to when preparing formulations without a hold step or the drop in Tm, density, number and size of Tmh crystals at 66° C.).

In some embodiments, formulations containing 1.2% hydrogenated castor oil with or without a combination of minocycline and adapalene are prepared by a holding process, where the holding step is performed at different temperatures. In some embodiments the holding temperature have a significant impact on the DSC pattern of the tested formulations and TM4 transition. In some embodiments formulations prepared at holding temperatures of about 54° C. and about 58° C. may increase TM4 (e.g., to about 69-70° C.). In some embodiments with holding temperatures of about 48° C. and about 66° C. does not produce significant changes in TM4.

Without being bound by theory, at lower holding temperatures (e.g., about 48° C.), the crystallization process may start prior to the holding. As a result, crystal nuclei may already have formed, allowing further crystallization seeded by those nuclei to proceed (whereas at a higher holding temperature a different crystallization process may take place). At higher holding temperatures (e.g., about 66° C.) which are closer to the melting temperature of hydrogenated castor oil in such formulations, the thermodynamic drive for a structural order of the molecules may be too low or the holding time of 4 hours at this temperature may be too short.

In some embodiments, the holding temperature is about 52° C., about 53° C., about 54° C., about 55° C., or about 56° C. and where TM4 is high. In some embodiments, holding temperature of about 54° C. and 56° C. may result in the presence of more plates and Tmh crystals, whereas holding temperatures of below 54° C. and above 56° C. may result in the presence of more spherulites that are associated with crystals of lower structural order. In one or more embodiments the holding temperature is between about 54-56° C.

In some embodiments, Tmh crystals are present in formulations prepared by holding temperatures of about 48° C. to about 58° C. In some embodiments, the relative percentage of area held by these Tmh crystals in formulations prepared by holding temperatures of about 48° C. to about 58° C. is above 70% compared to the relative percentage area held by the spherulites which is below 30%. In one or more embodiments, the holding temperature is between about 48° C. to about 58° C.

In some embodiments, Tmh crystals are bigger than spherulites independent of the holding temperature. In some embodiments, Tmh crystals are higher in number and cover a higher % of the area of the photomicrograph, except for the sample with a 58° C. holding temperature, where spherulites and Tmh crystals are present approximately in the same amount and % area. In some embodiments, Tmh crystals are not present in samples manufactured under 66° C. holding, while the samples with holding temperature of 54° C. and 56° C. exhibit the highest values for Tmh crystals (as measured by crystal count, size, and % Area). In some embodiments, the effect of holding temperature on Tmh crystals values is the same for both placebo formulations and formulations with active ingredient.

Without being bound by theory, a holding temperature for about 4 hours between 52° to 56° C. may result in high Tm4 values, which can be indicative of the presence of crystals of a higher structural order. This is confirmed by microscopy where more Tmh crystals are observed as compared to holding at above 58° C. or crystallizing below 48° C. which reduces the Tm4 values and reduces or eliminates formation of Tmh crystals. Likewise, localized overheating or overheating and then quick cooling may impact on the formation of Tmh crystals.

In some embodiments a formulation containing 1.2% hydrogenated castor oil, and a combination of minocycline and adapalene, prepared by a holding process with two holding steps first at 54° C. for 3 hours and then at 40° C. for 3 hours and stored at 40° C. for 15 days and 30 days show a slight increase in TM4 compared to formulation prepared with one hold step at 54° C. for 4 hours.

Without being bound by theory, a two-stage holding process may not negatively impact TM4. The Tmh crystals already formed in the first holding step may remain intact or consolidate during the second holding step. A second holding step at a lower temperature may e.g., affect crystallization of beeswax.

In some embodiments a formulation containing 1.2% hydrogenated castor oil, and a combination of minocycline and adapalene, prepared by a holding process with two holding steps at 54° C. for 3 hours and at 40° C. for 3 hours stored at 5° C. and 25° C. are shakable at all timepoints like formulations prepared with one hold step at 54° C. for 4 hours. In some embodiments, with a two-step process with shorter steps but overall longer holding shakability can go down when stored at 40° C.

In some embodiments, a formulation containing 1.2% hydrogenated castor oil and combination of minocycline and adapalene is prepared with a holding step at 54° C. for a longer holding process (about 16 hours) results in phase separation although the Tm4 and shakability profile is similar to that of shorter 4 hour holding step.

In some embodiments, formulations with an active agent, e.g., comprising a combination of minocycline and adapalene, are prepared using different holding temperatures and holding periods. In some embodiments, a formulation prepared with a holding step at 60° C. for 4 hr and shear is applied when additional components are introduced. In some embodiments, formulations are prepared with a holding step at 56° C. for 4 hr and 2 hr respectively. In some embodiments, a formulation is prepared with a shorter holding step at 54° C. (2 hr). In some embodiments, following 30 days of storage at 5° C. and 25° C. all formulations remain shakable, however at 40° C. only formulations generated by holding for 4 hr at 56° C. or 60° C. remain shakable, while the formulations with a shorter holding period of 2 hr either at 56° C. or 54° C. are non-shakable over time. In some embodiments, shakability is improved with a longer holding step at 56° C. (4 hr versus 2 hr) at 40° C. after 30 days and a higher TM4 similar to formulations with holding for 4 hr at 54° C.

In some embodiments, the TM4 value range is lower (e.g. 65.5-66.2° C.) where holding is shorter e.g., for 2 hr at 56° C. In some embodiments the TM4 value range is high (e.g., about 72° C.) where holding is for longer e.g., for 4 hr at 56° C. or for 4 hr at 60° C. (shear-additional components). In some embodiments, the TM4 values for 54C 2h and 56C 4 hr range is e.g., about 68-71° C. In some embodiments, the TM4 value for 2 hr at 56° C. is similar to formulations prepared with holding for 4 hr at 54° C.

In one or more embodiments holding is a single step. In some embodiments it is two or more steps. In some embodiments each holding step is at about the same temperature. In some embodiments each holding is at a successive lower temperature. In some embodiments the one or more of the later steps can be at a higher holding temperature, and in some embodiments such higher temperature steps should be below the melting temperature of the waxes in the emollient. In some embodiments the period of the holding step or combined holding steps is less than 16 hours, or less than 12 hours or less than 10 hours or less than 8 hours. In some embodiments they are between about 2 to about 8 hours. In some embodiments the proportion of Tmh crystals to spherulites and/or to plates can be modified by varying the holding time and/or the holding temperature. See e.g., Tables 18.

In some embodiments, the use of high shear during the holding step of the manufacturing process may be detrimental to the formation of Tmh crystals. In some embodiments, prolonged use of high shear may break or eliminate part or all of the Tmh crystals or enable part all of them to form other crystals. In some embodiments, after completion of the holding step the presence of high shear for a short period such as 10 mins during or after adding active agent in order to facilitate rapid formation of a homogenous mixture does not appear to have a marked or detrimental effect on the Tmh crystals.

In some embodiments, the use of low shear during the holding step may not prevent the formation of Tmh crystals although prolonged use of low shear may break or eliminate some Tmh crystals or enable some to form other crystals

In some embodiments, the holding process comprises the step of cooling the mixture to a set temperature, e.g., about 54° C., and holding at that temperature for fixed time, e.g., about 4 hours, i.e., a holding step at the fixed temperature. In some embodiments, the holding step is a single hold. In some embodiments the holding step involves two or more holds (e.g., a first hold and a second hold). In some embodiments the first hold is at a higher temperature (e.g. about 56° C. for a fixed time with mixing) and at the end of the fixed time (e.g. about 3 hours) cooling with mixing to a second hold at a lower temperature (e.g. about 52° C. for a fixed time with mixing) and at the end of that fixed time (e.g. about 3 hours) continuing with step (c) above. In some embodiments the first hold is at a lower temperature (e.g. about 52° C. for a fixed time with mixing) and at the end of the fixed time (e.g. about 3 hours) warming with mixing to a second hold at a higher temperature (e.g. about 56° C. for a fixed time with mixing) and at the end of that fixed time (e.g. about 3 hours) continuing with step (c) above. In one or more embodiments there are three holds. In some embodiments each hold is at a lower temperature than the previous one. In some embodiments the reverse. In some embodiments the first is at a higher temperature, the second at a lower temperature and the third at a higher temperature, which is higher than the second but is lower or no higher than the first. In some embodiments the first is at a lower temperature, the second at a higher temperature and the third at a lower temperature, which is lower than the second but is lower or no higher than the first.

In some embodiments, the holding step is done at about 48-66° C., e.g., about 52-58° C.. In some embodiments, the holding step is done at about 48° C. to about 60° C., for example, about 48° C. to about 59° C., about 58° C., about 57° C., about 56° C., about 55° C., about 54° C., about 53° C., about 52° C., about 51° C., about 50° C.; or about 49° C. to about 59° C., about 58° C., about 57° C., about 56° C., about 55° C., about 54° C., about 53° C., about 52° C., about 51° C., or about 50° C. or can be a range between any two figures listed in this paragraph, such as 54-58° C..

In one or more embodiments, the holding step is conducted for about 1 to 72 hours, e.g., about 66 hours, about 60 hours, about 54 hours, about 48 hours, about 42 hours, about 1 to 36 hours, about 1 to 30 hours, about 1 to 24 hours, about 1 to 18 hours, about 1 to 16 hours, about 1 to 12 hours, about 1 to 10 hours, about 1 to 8 hours, about 1 to 6 hours, about 1 to 5 hours, about 1 to 4 hours, about 1 to 3 hours, about 1 to 2 hours, about 2 to 4 hours, or about 4 to 6 hours. In one or more embodiments, the holding step is conducted for about 1-12 hours. In some other embodiments the holding step may be longer.

In some embodiments, the holding step may allow for minor temperature fluctuation during the holding period. In some embodiment, the holding step allows the temperature to drop or rise slowly, e.g. a rise or a drop in temperature of about 10° C./10 hours, 10° C./5 hours, or 10° C./2 hours. In some embodiments, during the holding step the temperature is dropped slowly (for e.g. 10° C./10 hours, 10° C./5 hours, or 10° C./2 hours) then raised slowly (for e.g. 10° C./10 hours, or 10° C./5 hours, or 10° C./2 hours) then dropped slowly (for e.g. 10° C./10 hours, 10° C./5 hours, or 10° C./2 hours) again. In some embodiments the holding step comprises a fixed temperature holding step followed by a gradient holding step. In some embodiments the holding step comprises a gradient holding step followed by a fixed temperature holding step.

In some embodiments, during and/or after the cooling and hold steps, mixing using high shear or other methods that can input heat or energy into the crystals e.g., as they pass through a homogenizer mixer, may be avoided or ameliorated or used only for a relatively short period and/or at lower speed so that the Tmh crystals are not substantially reduced or eliminated in the composition.

In some embodiments the method comprises mixing a wax (e.g. hydrogenated castor oil) with one or more oils (e.g. soybean oil) and then performing the holding process only on this mixture. In one or more embodiments, the method comprises performing a holding process for a mixture of oil(s) and each of the multiple waxes separately. In one or more embodiments the method comprises performing a separate holding process for a mixture of oil(s) and foam adjuvants and/or other excipients. In one or more embodiments, the method comprises performing a separate holding process for mixtures of oil(s) and each of multiple waxes and for a mixture of oil(s) and the foam adjuvants and/or other excipients followed by mixing the components and performing holding of the resultant mixture.

Also provided herein are methods of preparing a foamable composition comprising a tetracycline antibiotic, at least one emollient, at least one wax e.g., hydrogenated castor oil; or comprising a tetracycline antibiotic, at least one emollient, at least one foam adjuvant, and a wax e.g., hydrogenated castor oil. In some embodiments, the method comprises (a) mixing at least one emollient, at least one foam adjuvant, and a wax such as hydrogenated castor oil, and heating the mixture to a temperature sufficient to completely melt the mixture or to a temperature at which a homogenous mixture can be observed; (b) cooling the mixture to a temperature of about 54° C., and holding at that temperature for about 4 hours; (c) cooling the mixture to a temperature of about 35° C. to about 40° C.; (d) adding the active agent such as a tetracycline antibiotic and/or an emollient such as cyclomethicone, to the mixture; (e) cooling the mixture to a temperature of about 24° C. to about 28° C.; (f) optionally adding an additional active agent such as a retinoid to the mixture; (g) cooling the mixture to a temperature of about 22° C. to about 28° C.; and (h) stirring the mixture for up to 24 hours at a temperature of about 20° C. to about 24° C.. In some embodiments, the process comprises the step of cooling the mixture to a set temperature, e.g., about 54° C., and holding at that temperature for fixed time, e.g., about 4 hours. In some embodiments, the process comprises the step of cooling the mixture to a set temperature, e.g., about 54° C., and holding at that temperature for fixed time, e.g., about 2 hours. In some embodiments, the process comprises the step of cooling the mixture to a set temperature, e.g., about 56° C., and holding at that temperature for fixed time, e.g., about 6 hours. In some embodiments, the process comprises a holding step (e.g., as described above and herein).

In one or more embodiments the mixture is cooled to room temperature. In one or more embodiments the cooling is with mixing. In some embodiments it is cooled to about 20° C. to about 28° C., or about 22° C. to about 28° C., or about 24° C. to about 28° C., or about 20° C. to about 26° C., or about 20° C. to about 25° C., or about 20° C. to about 24° C., or about 20° C. to about 23° C., or about 20° C. to about 22° C., or about 21° C. to about 26° C. about 22° C. to about 26° C., or about 23° C. to about 26° C., or about 24° C. to about 26° C., about 21° C. to about 25° C., or about 22° C. to about 25° C., or about 23° C. to about 25° C., or about 22° C. to about 24° C., or about 20° C., or about 21° C., or about 22° C., or about 23° C., or about 24° C., or about 25° C., or about 26° C., or about 27° C., or about 28° C.. In one or more embodiments cooling is to about to 35° C. to about 40° C., and then to about 24° C. to about 28° C., at which ranges, if they are to be included, sensitive active agents are added, as appropriate (e.g., according to their sensitivity), and mixed so they are homogenous. In one or more embodiments mixing to homogeneity is for up to an hour or about an hour. In one or more embodiments mixing to homogeneity is more than an hour (e.g., about 1-2 hours). In some embodiments the formulation is then further subjected to cooling to about 22° C. to about 28° C., or about 22° C. to about 26° C., In some further embodiments it is to about 22° C. to about 24° C., or about 20° C. to about 26° C., In some embodiments the formulation is then subjected to a mixing step for about 3 up to 24 hours. In some embodiments the mixing step that follows cooling may start at about 28° C. with cooling continuing to between about 20° C. to about 24° C. and then maintaining the temperature in that range. In some embodiments the mixing step that follows cooling may start at about 27° C., or about 26° C., or about 25° C. with cooling continuing to between about 20° C. to about 24° C. and then maintaining the temperature in that range. In some embodiments the mixing step that follows cooling may start at about 24° C., or at about 23° C., at about 22° C., with the temperature being maintained in the range of about 20° C. to about 24° C.. In some embodiments the temperature is maintained in the range of about 20° C. to about 26° C. In some embodiments the temperature is maintained at about 20° C., or about 21° C., or about 22° C., or about 23° C., or about 24° C..

Also provided herein are methods of preparing a foamable composition comprising a tetracycline antibiotic, a retinoid, at least one emollient, at least one foam adjuvant, and a wax e.g., hydrogenated castor oil. In some embodiments, the method comprises (a) mixing at least one emollient, at least one foam adjuvant, and hydrogenated castor oil, and heating the mixture to a temperature sufficient to completely melt the mixture; (b) cooling the mixture to a temperature of about 54° C., and holding at that temperature for about 4 hours; (c) cooling the mixture to a temperature of about 35° C. to about 40° C.; (d) adding the tetracycline antibiotic and/or cyclomethicone, to the mixture; (e) cooling the mixture to a temperature of about 24° C. to about 28° C.; (f) adding the retinoid to the mixture; (g) cooling the mixture to a temperature of about 22° C. to about 24° C.; and (h) stirring the mixture for up to 24 hours at a temperature of about 20° C. to about 26° C.. In some embodiments, the process comprises the step of cooling the mixture to a set temperature, e.g., about 54° C., and holding at that temperature for fixed time, e.g., about 4 hours. In some embodiments, the process comprises the step of cooling the mixture to a set temperature, e.g., about 54° C., and holding at that temperature for fixed time, e.g., about 2 hours. In some embodiments, the process comprises the step of cooling the mixture to a set temperature, e.g., about 56° C., and holding at that temperature for fixed time, e.g., about 6 hours. In some embodiments, the process comprises a holding step (e.g., as described herein).

In some embodiments, the method further comprises a step of pressurizing the mixture with a propellant, e.g., Propellant AP-70, in an aerosol container.

In some embodiments, foamable compositions are packed in a container with an outlet valve e.g., aerosol canister. Possible containers and valves are likewise described in the literature as known by those skilled in the art.

According to another aspect, both the minocycline and the foamable compositions containing minocycline can be manufactured under current Good Manufacturing Principles (cGMP) conditions. In some embodiments, the foamable composition is provided in aluminum aerosol canisters mounted with valve and actuator. In some embodiments the size of the cannister is determined by the dose to be delivered. In some embodiments the cannister may provide doses for 3 months, or 2 months, or 1 month, or for one or two weeks. In some embodiments the cannister may be a single dose cannister. In some embodiments, a canister is filled with 35 g of product and 4.2 g of propellant. In some embodiments, the canister is filled with 15 gr product and 1.8 gr propellant. In some embodiments, the canister is filled with 6 gr product and 0.72 gr propellant. In some embodiments, the canister is filled under nitrogen atmosphere. Upon actuation of the canister an aliquot of quality foam is released.

### Composition Structure and the Influence of Crystals

In some embodiments, the composition and/or foamable composition comprises crystals. Without being bound by theory, hydrogenated castor oil and/or other excipients such as other waxes (alone or combinations) may form crystals or co-crystals within the composition and/or foamable composition and/or released foam, that have different shapes, e.g., nonuniform, rod, plate, needle, or sphere. Formulation components, e.g., waxes, that are solid at room or body temperatures, may form microcrystals distributed homogenously within the foamable composition. In some embodiments, crystals, e.g., microcrystals or large crystals, are distributed homogenously within the foamable composition. The foamable compositions and the methods to prepare them described herein, may affect the shapes of the crystals within the compositions. The shapes of the crystals in turn may affect the stability, viscosity, rheology, sebum softening/breakdown/liquification, melting properties, and/or shakability of the compositions, foamable compositions, and/or foams. This disclosure is based partly on the surprising discovery that altering the crystal fingerprint of the formulation, e.g., crystal fingerprint of the components that are solid at body temperature, e.g., waxes or hydrogenated castor oil, can improve the stability, usability, flowability, shakability of the foamable compositions and/or foams.

The formulations provided herein may comprise a mixture of components, each with its own melting temperature and crystallization profile. Without being bound by theory, each component of the formulations provided herein can affect the crystallization profiles, melting temperature, and/or crystallization fingerprint of other components within the formulation. In one or more embodiments, the combination of different excipients and/or active agents at different amounts in the formulations provided herein forms crystals with a unique crystallization fingerprint. The crystallization fingerprint may affect the properties of the formulations, e.g. sebum liquification, fluidity, shakability, and stability.

In some embodiments, a formulation provided herein has Tmh crystals. In some embodiments, even a relatively small proportion of such crystals, relative to the overall composition, may surprisingly improve stability (e.g., storage stability for a period of days, months, years) at room temperature. In some embodiments, such a formulation provides surprisingly improved stability even when the formulation comprises an active agent or other excipient which can destabilize the formulation and/or interfere with shockability (e.g., adapalene). Such formulations with Tmh crystals may also improve the ability of the formulation to melt sebum and thereby increase penetration.

In some embodiments, Tmh crystals are formed from hydrogenated castor oil (or other waxes) during a holding step process disclosed herein. In some embodiments, Tmh crystals are co-crystals of hydrogenated castor oil (or other waxes) and other excipients during a holding step process disclosed herein.

In one or more embodiments, the formulations disclosed herein, e.g., foam formulations, comprise crystals with a crystal fingerprint that is associated with preparation of the formulation by a holding process. In one or more embodiments, foamable formulations comprising propellant comprise crystals with a crystal fingerprint that is associated with preparation of the formulation by a holding. In one or more embodiments, foam formulations not comprising propellant (i.e., after foam formation) comprise crystals with a crystal fingerprint that is associated with preparation of the formulation by a holding. In one or more embodiments a crystal fingerprint in a foamable formulation is also present in the resultant foam.

In some embodiments, some or all (e.g., a majority) of the crystals in a composition, foamable composition, and/or foam are Tmh crystals (e.g., crystals having nonuniform shapes formed during a holding process method disclosed herein). In some embodiments, some or all the crystals are spherulites. In some embodiments, some or all the crystals are plate shaped. In some embodiments, some or all the crystals are needle shaped. In some embodiments, some or all the crystals are rod-shaped. In some embodiments, the crystals have a combination of shapes, e.g., more than one of nonuniform, rod, plate, needle, sphere, or spherulite shapes. In some embodiments, a majority of the crystals are nonuniform, e.g., not spheroid. In some embodiments, a majority of the crystals comprise nonuniform shapes. In some embodiments, a majority of the crystals comprise spherulite shapes. In some embodiments, a majority of the crystals comprise plate shapes. In some embodiments, a majority of the crystals comprise needle shapes. In some embodiments, a majority of the crystals comprise rod shapes. In some embodiments, less than a majority of the crystals comprise spherulites. In some embodiments, the crystals in a foamable composition comprise nonuniform shapes to a greater degree than in a comparable composition and/or foamable composition or foam produced by a process that does not comprise a hold step, e.g., a step of cooling the mixture to a temperature of about 50-60° C. and maintaining the mixture at the temperature for about 1-12 hours.

In one or more embodiments, the ratio of Tmh crystals to spherulites and plates is about 100:1, e.g., about 95:1, about 90:1, about 85:1, about 80:1, about 75:1, about 70:1, about 65:1, about 60:1, about 55:1, about 50:1, about 45:1, about 40:1, about 35:1, about 30:1, about 25:1, about 20:1, about 15:1, about 10:1, about 5:1, about 1:1, about 1:5, about 1:10, about 1:15, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, about 1:100. In some embodiments the ratio is more than any of the aforesaid. In some other embodiments the ratio is less than any of the aforesaid, By way of example, in an embodiment more than 100:1, and in another embodiment less than 100:1.

In one or more embodiments, the ratio of Tmh crystals to spherulites is about 100:1, e.g., about 95:1, about 90:1, about 85:1, about 80:1, about 75:1, about 70:1, about 65:1, about 60:1, about 55:1, about 50:1, about 45:1, about 40:1, about 35:1, about 30:1, about 25:1, about 20:1, about 15:1, about 10:1, about 5:1, about 1:1, about 1:5, about 1:10, about 1:15, about 1:20, about 1:25, about 1:30, about 1:35, about 1:40, about 1:45, about 1:50, about 1:55, about 1:60, about 1:65, about 1:70, about 1:75, about 1:80, about 1:85, about 1:90, about 1:95, about 1:100. In some embodiments the ratio is more than any of the aforesaid. In some other embodiments the ratio is less than any of the aforesaid, By way of example, in an embodiment more than 100:1, and in another embodiment less than 100:1.

In some embodiments, the Tmh crystals formed in formulations prepared by a holding process have an average cross-sectional area of about 40 to about 150 μm², or about 50 to about 150 μm².

In some embodiments, the Tmh crystals formed in formulations prepared by a holding process have an average cross-sectional area of about 50-80 μm² on average (e.g., about 55-70 μm² on average, e.g., about 61-63 μm² on average). In some embodiments, the Tmh crystals formed in formulations prepared by a holding process are larger than the spherulites observed in formulations prepared by a continuous heating-cooling process. In some embodiments, cross-sectional area is measured at the largest cross-sectional point in a crystal.

In one or more embodiments, the Tmh crystals prepared by a holding process occupy about or at least about 15% of the area measured in a representative image of a sample of the formulation, taken by observing the sample, e.g., a 1 mm³, 3 mm³, or 5 mm³ volume, under a light microscope, e.g., wherein the image is about 650×450 μm, e.g., 645×452 μm. In some embodiments, the percentage of the area occupied by the Tmh crystals is compared to that for spherulites. In some embodiments the Tmh crystals occupy by area about 16%, about 17%, about 18%, about 19%, about 20%, about 21%, about 22%, about 23% about 24%, about 25%, about 26%, about 27%, about 28%, about 29% %, about 30%, about 31%, about 32% %, about 33%, about 34%, or about 35% of the area measured. In some embodiments the Tmh crystals occupy about e.g., about 15% to about 35%, about 20% to about 34%, about 25% to about 33%, or about 30% to about 33% of the area measured.

In some embodiments, the percentage area occupied by spherulites is measured using one or more of the methods discussed above for measuring the percentage area of Tmh crystals in a formulation sample. In one or more embodiments, spherulites observed in formulations prepared by a continuous heating-cooling process have an average area of about 24-25 μm². In some embodiments, the spherulites observed in formulations prepared by a continuous heating-cooling process are on average smaller than the Tmh crystals formed in formulations prepared by a holding process, e.g., having cross-sectional area of about 50-80 μm² on average (e.g., about 55-70 μm² on average, e.g., about 61-63 μm² on average). In one or more embodiments, the spherulites occupy about 20-25%, e.g., about 22-23% of the area measured in a sample.

In some embodiments, the percentage area occupied by plates is measured using one or more of the methods discussed above for measuring the percentage area of Tmh crystals in a formulation sample. In one or more embodiments, crystals with plate structures are present in a formulation disclosed herein. In some embodiments, plates are observed whether the composition is prepared according to a holding or according to a continuous process. In some embodiments, crystals with plate structures are smaller on average in formulations prepared by a continuous heating-cooling process (about 12-13 μm² on average) compared to those prepared by a holding process (about 15-19 μm² on average). In one or more embodiments, crystals with plate structures have an average size of about 10-20 μm², e.g., about 12-13 μm², in formulations prepared by a continuous heating-cooling process. In one or more embodiments, crystals with plate structures have an average size of about 15-19 μm² in formulations prepared by a holding process.

In one or more embodiments, the holding process does not substantially change the crystal polymorph of a formulation disclosed herein. In one or more embodiments, the holding process forms a new polymorph that is very similar, e.g., having subtle differences, to the polymorph formed by a process other than a holding process. In one or more embodiments the crystals in a formulation prepared with a hold step have a different microstructure as compared to crystals formed by a process other than a holding process. In some embodiments there is no observed change in polymorph by x-ray powder diffraction but there is in crystal structure. In some embodiments there is a change in polymorph and in crystal structure. In one or more embodiments, the holding process changes crystal morphology, e.g., changes in crystal microstructure to alter the shape and/or concentration of Tmh crystals, but does not change crystal polymorph, e.g., crystal lattice.

In some embodiments, the method described herein of preparing a foamable composition produces a foam that comprises more crystals with nonuniform shapes as compared to other structures, e.g., fibers, needles, plates, rods, or a combination thereof, particularly crystals or microcrystals with regular shapes, e.g., spherulites, as measured by transmission electron microscopy. In some embodiments, the method described herein of preparing a foamable composition produces a foam that comprises more crystals with nonuniform shapes, as measured by light microscopy, transmission electron microscopy (TEM), Differential Scanning Calorimetry (DSC), small angle X-Ray scattering, wide angle X-Ray scattering, Field Emission Scanning Electron Microscopy and Energy Dispersive X-ray Spectroscopy (FE-SEM/EDS) and/or infrared spectrometry. In some embodiments, DSC is used. In some embodiments, X-ray powder diffraction is used.

Differential scanning calorimetry, or (DSC) is a thermoanalytical technique in which the difference in the amount of heat required to increase or decrease the temperature of a sample and reference is measured as a function of temperature. Without being bound by theory, by observing the difference in heat flow between a sample and reference, differential scanning calorimeters are able to measure the amount of heat absorbed or released at a specific temperature during phase transitions (Tm). In one or more embodiments, the compositions provided herein comprise Tmh crystals with a DSC pattern comprising a phase transition temperature T_{M4} of about 50-80° C., e.g., above 60° C., or above 64° C., e.g., 68-69° C. or e.g., about 64-72° C., e.g., about 68-72° C.. In one or more embodiments, the compositions provided herein comprise Tmh crystals with a DSC pattern comprising a phase transition temperature T_{M4} that is about 3° C. or about 4° C., or about 5° C., or about 6° C. higher than that of a formulation prepared in a continuous heating-cooling process. In some embodiments, the use of HCO contributes to the higher T_{M4}, e.g., alone or in combination with a holding process step.

In one or more embodiments, Tmh crystals in a formulation disclosed herein are measured by small angle X-ray scattering. In some embodiments, the average intensity of the crystals in a formulation prepared by a hold process, as measured by small angle X-ray scattering, is about 0.024 to 0.05 cm⁻¹ at 2θ=0.7-1.2. In some embodiments, the average intensity as measured by small angle X-ray scattering for a formulation prepared by a holding process at 20=0.7-1.2 is about 0.024 cm⁻¹, about 0.025 cm⁻¹, about 0.026 cm⁻¹, about 0.027 cm⁻¹, about 0.028 cm⁻¹ about 0.029 cm⁻¹, about 0.030 cm⁻¹, about 0.031 cm⁻¹, about 0.032 cm⁻¹, about 0.033 cm¹, about 0.034 cm⁻¹, about 0.035 cm⁻¹, about 0.036 cm⁻¹, about 0.037 cm⁻¹, about 0.038 cm⁻¹, about 0.039 cm⁻¹, about 0.040 cm⁻¹, about 0.041 cm⁻¹, about 0.042 cm⁻¹, about 0.043 cm⁻¹, about 0.044 cm⁻¹, about 0.0405 cm⁻¹, about 0.046 cm⁻¹, about 0.047 cm⁻¹, about 0.048 cm⁻¹, about 0.049 cm⁻¹, or about 0.050 cm⁻¹.

In one or more embodiments, the average intensity as measured by small angle X-ray scattering for a formulation prepared by a continuous heating-cooling process is about 0.005 to about 0.02 cm⁻¹ at 2θ=0.7-1.2. In some embodiments, the average intensity as measured by small angle X-ray scattering for a formulation prepared by a continuous heating-cooling process at 2θ=0.7-1.2 is about 0.005 cm⁻¹, about 0.006 cm⁻¹, about 0.007 cm⁻¹, about 0.008 cm⁻¹, about 0.009 cm⁻¹, about 0.010 cm⁻¹, about 0.011 cm⁻¹, about 0.012 cm⁻¹, about 0.013 cm⁻¹, about 0.014 cm⁻¹, about 0.015 cm⁻¹, about 0.016 cm⁻¹, about 0.017 cm⁻¹, about 0.019 cm⁻¹, or about 0.020 cm⁻¹.

In one or more embodiments, the average intensity as measured by small angle X-ray scattering for a formulation prepared by a holding process is about 0.031 cm⁻¹ versus about 0.014 cm⁻¹ for a formulation prepared by a continuous heating-cooling process.

In one or more embodiments, the average intensity as measured by small angle X-ray scattering of a formulation prepared by a holding process is more than 2 times higher, more than 2.5 times higher, more than 3 times higher, more than 3.5 times higher, more than 4 times higher, more than 4.5 times higher, more than 5 times higher, more than 5.5 times higher, more than 6 times higher, more than 6.5 times higher, more than 7 times higher, more than 7.5 times higher, more than 8 times higher, more than 8.5 times higher, more than 9 times higher, more than 9.5 times higher, or more than 10 times higher than that of a formulation prepared by a continuous heating-cooling process.

As used herein, a “unit cell” refers to the smallest repeating unit in a crystal. In some embodiments, Tmh crystals have stronger interactions between unit cells than regular-shaped crystals, e.g., rods, plates, needles. In some embodiments, Tmh crystals have unit cells that are tangled fibers.

Also contemplated is a method of increasing the stability of wax crystals in a foam composition. In some embodiments, the method comprises: (a) formulating a mixture comprising at least one emollient, at least one foam adjuvant, and a wax, such as hydrogenated castor oil; (b) heating the mixture to a first temperature sufficient to completely melt the at least one emollient, the at least one foam adjuvant, and wax; (c) cooling the mixture in a nonlinear fashion that comprises holding at a second temperature; and (d) stirring the mixture at the second temperature; wherein the first temperature is higher than the second temperature. In some embodiments, the first temperature is about 10-90° C. higher than the second temperature. In some embodiments, the first temperature is about 10-60° C. higher than the second temperature. In some embodiments, the first temperature is about 10-40° C. higher than the second temperature. In some embodiments, the second temperature is about 45-70° C., e.g., about 49° C., about 50° C., about 51° C., about 52° C., 53° C., about 54° C., about 55° C., about 56° C., about 57° C., 58° C., about 59° C., about 60° C., about 61, about 62° C., about 63° C., about 64° C., or about 65° C.. In some embodiments, holding at a second temperature is for at least about 20 minutes to about 24 hours, e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 16 hours, about 18 hours, about 20 hours, or about 22 hours.

In some embodiments, Tmh wax crystals obtained from a holding process are present in the foamable formulations provided herein before addition of propellant. In some embodiments, the Tmh wax crystal are present in the foamable formulations comprising propellant provided herein. In some embodiments the Tmh crystals are present in the resultant foam obtained from the foamable formulations provided herein. In one or more embodiments the Tmh crystals are present in the foamable formulations before and after addition of propellant. In one or more embodiments the Tmh crystals are present in the foamable formulations and in the resultant foam.

### Sebum

Sebum is a light yellow, oily substance that is secreted by the sebaceous glands that help keep the skin and hair moisturized. The ability of the active agents in the foam or foamable compositions to be absorbed by the skin may depend on their ability to mix with sebum and/or penetrate into the pilosebaceous unit. In one or more embodiments compositions, and or foamable compositions and or foams described herein surprisingly reduce the melting temperature of sebum and or aid its dissolution, liquefaction or break-up. Without being bound by theory, dissolution or liquefaction of the sebum may decrease the viscosity of sebum at skin temperature, increase the diffusion coefficient and provide for better ability of the active ingredient to penetrate into pilosebaceous ducts. Again, without being bound by theory, by aiding in the break-up of sebum, the formulations disclosed herein may help to open up the pores and provide deliver therapeutic agents, e.g., minocycline and adapalene, into pilosebaceous ducts for treatment of inflammatory and non-inflammatory lesions. Without being bound by theory, the crystal fingerprint present in formulations prepared by a holding process comprises Tmh crystals that are unique and may have reduced crystal interaction with other components of the formulation, resulting in a higher formulation flowability and different formulation melting temperatures and enthalpy, that may improve the composition's ability to dissolve or break down sebum.

In some embodiments, the foamable composition is capable of softening sebum, which in turn may help increase skin penetration. In some embodiments, a foamable formulation prepared by a holding process reduces sebum melting point to a greater extent than an oil in water emulsion. In one or more embodiments, the foamable composition provided herein reduces sebum melting point to a greater extent than a formulation prepared in a continuous heating-cooling process. In one or more embodiments, a foamable composition prepared in a holding process reduces sebum melting point to a greater extent than a formulation prepared in a continuous heating-cooling process. In one or more embodiments, the foamable composition provided herein reduces sebum melting point temperature by about 2° C., about 3° C., about 4° C., about 5° C., about 6° C., about 7° C., or about 8° C.

In some embodiments, the melting temperature of a 1:1 mixture of sebum and a foamable composition disclosed herein is 10% lower than the melting temperature of sebum alone. In some embodiments, the melting temperature of a 1:1 mixture of sebum and the foamable composition is 20% lower than the melting temperature of sebum alone. In some embodiments, the melting temperature of a 1:1 mixture of sebum and the foamable composition is 30% lower than the melting temperature of sebum alone. In some embodiments, the melting temperature of a 1:1 mixture of sebum and the foamable composition is 40% lower than the melting temperature of sebum alone. In some embodiments, the melting temperature of a 1:1 mixture of sebum and the foamable composition is 50% lower than the melting temperature of sebum alone. In some embodiments, the melting temperature of a 1:1 mixture of sebum and the foamable composition is 60% lower than the melting temperature of sebum alone. In some embodiments, the melting temperature of a 1:1 mixture of sebum and the foamable composition is 70% lower than the melting temperature of sebum alone. In some embodiments, the melting temperature of a 1:1 mixture of sebum and the foamable composition is 80% lower than the melting temperature of sebum alone. In some embodiments, the melting temperature of a 1:1 mixture of sebum and the foamable composition is 90% lower than the melting temperature of sebum alone. In some embodiments, the melting temperature of a 1:1 mixture of sebum and the foamable composition is 100% lower than the melting temperature of sebum alone.

In some embodiments, the melting temperature of a 1:1 mixture of sebum and a foamable composition disclosed herein prepared by a holding process is lower than that of the same composition prepared by a continuous heating-cooling process. In some embodiments, the melting temperature of a 1:1 mixture of sebum and a foamable composition disclosed herein prepared by a holding process is about 10% lower, about 15% lower, about 20% lower, about 25% lower, about 30% lower, about 35% lower, about 40% lower, about 45% lower, about 50% lower, about 55% lower, about 60% lower, about 65% lower, about 70% lower, about 75% lower, about 80% lower, about 85% lower, about 90% lower, about 95% lower, or about 100% lower than the melting temperature of a 1:1 mixture of sebum an a foamable composition prepared by a continuous heating-cooling process.

In some embodiments, wherein the melting point of the 1:1 mixture of sebum and the foamable composition is between about 30.0° C. and 31.0° C. In some embodiments, wherein the melting point of the 1:1 mixture of sebum and the foamable composition is between about 31.0° C. and 32.0° C. In some embodiments, wherein the melting point of the 1:1 mixture of sebum and the foamable composition is between about 32.0° C. and 33.0° C. In some embodiments, wherein the melting point of the 1:1 mixture of sebum and the foamable composition is between about 33.0° C. and 34.0° C. In some embodiments, wherein the melting point of the 1:1 mixture of sebum and the foamable composition is between about 34.0° C. and 35.0° C. In some embodiments, wherein the melting point of the 1:1 mixture of sebum and the foamable composition is between about 35.0° C. and 36.0° C. In some embodiments, wherein the melting point of the 1:1 mixture of sebum and the foamable composition is between about 36.0° C. and 37.0° C. In some embodiments, wherein the melting point of the 1:1 mixture of sebum and the foamable composition is between about 37.0° C. and 38.0° C. In some embodiments, wherein the melting point of the 1:1 mixture of sebum and the foamable composition is between about 38.0° C. and 39.0° C. In some embodiments, wherein the melting point of the 1:1 mixture of sebum and the foamable composition is between about 39.0° C. and 40.0° C. In some embodiments, wherein the melting point of the 1:1 mixture of sebum and the foamable composition is between about 40.0° C. and 41.0° C. In some embodiments, wherein the melting point of the 1:1 mixture of sebum and the foamable composition is between about 41.0° C. and 42.0° C. In some embodiments, wherein the melting point of the 1:1 mixture of sebum and the foamable composition is between about 42.0° C. and 43.0° C.

### Foam

Foam is advantageous in the topical treatment of skin diseases since it is light and easy to apply and collapses and spreads with a minor mechanical force like a simple rub. When dispensed, even in small quantities, drug delivery in the form of foam can also cover a larger surface area of application while also facilitating better product application in areas where conventional topical products cannot be as effective. Foam absorbs rapidly—without the need of repeated rubbing—which is helpful and important for treatment of damaged or irritated skin, sores, and lesions. As the composition is absorbed quickly, this can contribute to a positive treatment effect by the vehicle alone, or when in combination with the active agent, a higher percentage effect by the active agent may be observed.

Stable foam which breaks upon application of mild shear force is extremely advantageous in the topical treatment of skin diseases. It can be applied directly onto skin or hands of the patient without collapsing. The foamable compositions described herein facilitate easy application and even distribution of the active agent, thereby improving treatment convenience.

The formulation packaged into an aerosol container is devoid of any contact with air, light, or any other form of contamination (e.g., moisture) as it is a completely sealed system throughout the life of the product. Thus, light and oxidation sensitive active agents can be effectively stabilized in the aerosol system. In some embodiments, the composition and/or foamable compositions described herein are filled into aerosol cans or canisters and pressurized with a propellent. The aerosol can or canister may comprise an outlet valve, that when actuated, releases the foamable composition from the can or canister and converts it into a foam composition. In some embodiment, the aerosol or canister comprises a nozzle for directing a foamable composition from the inside to the outside of the canister.

The foamable compositions disclosed herein can be applied to the target site as a gel or a semi-solid gel or ointment or foam or mousse. In some embodiments, the foamable compositions can be applied as a liquid gel or as a collapsed foam. In one or more embodiments, the composition is thixotropic. In some embodiments, the foamable formulation shows a reduction in viscosity with time when subjected to constant shear force. In some embodiments, after the foamable formulation is allowed to rest for a period of time, the viscosity increases again. In some embodiments, the foamable composition is a solid or semi-solid composition or gel prior to adding a propellant. In some embodiments, the foamable composition is a gel or a liquid. In some embodiments the propellant is miscible with the foamable composition. In some embodiments the propellant dilutes the foamable composition.

Upon packaging of the foamable composition in an aerosol container and adding a propellant, a shakable and homogenous foamable composition is formed. In some embodiments, the foamable composition forms a breakable foam with good to excellent quality when dispensed. Since the propellant evaporates during dispensing, the resulting foam is pharmaceutically equivalent to the foamable composition (prior to adding the propellant). Thus, upon collapse, the foam is compositionally similar or identical to the foamable composition. This is an advantage as the drug development process, including toxicology studies and clinical trials, need only be conducted for either the foamable composition or the foam and not for both.

In one or more embodiments, the foamable composition when packaged in an aerosol container to which is added a liquefied or compressed gas propellant, the foamable composition provides upon release from the container a breakable foam of at least good quality that breaks easily upon application of mechanical force.

In one or more embodiments, the foamable composition is thermally stable at skin temperature (e.g., about 37° C.).

In one or more embodiments, the foamable composition is filled into an aerosol can or canister and pressurized with a propellant.

In one or more embodiments, a foamable composition provided herein comprises: (a) about 60% to about 95% by weight of at least one emollient; (b) about 1% to about 2% by weight of hydrogenated castor oil; (c) about 0.1% to about 5% by weight of a tetracycline antibiotic (e.g., minocycline or minocycline HCl); (d) about 0.1% to about 1% by weight of a retinoid; and (e) about 5% to about 15% by weight at least one foam adjuvant.

In one or more embodiments, a foamable composition provided herein comprises: (a) about 60% to about 95% by weight of at least one emollient; (b) about 1% to about 2% by weight of hydrogenated castor oil; (c) about 0.1% to about 5% by weight of a tetracycline antibiotic (e.g., minocycline or minocycline HCl); (d) about 0.1% to about 1% by weight of a retinoid; (e) about 5% to about 15% by weight at least one foam adjuvant; and (f) about 1% to about 5% by weight at least one wax foam adjuvant.

In one or more embodiments, the compositions provided or described herein comprise a carrier and a propellant. In one or more embodiments, the carrier comprises a foamable composition provided or described herein.

In one or more embodiments, the foamable composition is a gel, paste, lotion, spray, mask, patch, pomade, ointment, oil, foam or mousse. In one or more embodiments, the foamable composition is hydrophobic. In one or more embodiments, the foamable composition comprises hydrophobic oils and waxes. In one or more embodiments, the foamable composition comprises fatty alcohols. In one or more embodiments, the foamable composition comprises hydrophobic oils and fatty alcohols. In one or more embodiments, the foamable composition comprises fatty acids. In one or more embodiments, the foamable composition comprises hydrophobic oils and fatty acids.

In one or more embodiments, the foamable composition comprises a gelled oil. In one or more embodiments, the gelled oil is a gelled mineral oil. In one or more embodiments, the gelled mineral oil is a VERSAGEL®. VERSAGELs® are gelled oils or emollients that can come in different product forms including, for example, the VERSAGEL® m, VERSAGEL® p, VERSAGEL® r, and VERSAGEL® s series, and provide various viscosity grades. There are also VERSAGELs® with isohexadecane, or with isododecane, or with hydrogenated polyisobutene, or with isopropylpalmitate. In an embodiment, it is VERSAGEL® 750 m. In an embodiment, it is VERSAGEL® 200 m. In an embodiment, it is VERSAGEL® 500 m. In an embodiment, it is VERSAGEL® 1600 m. VERSAGEL® m contains a mixture of mineral oil plus one or two or more of e.g., Ethylene/Propylene/Styrene Copolymer plus e.g., Butylene/Ethylene/Styrene Copolymer plus e.g., butylated hydroxyl toluene or similar gelling agents.

Once formed, the stability of a foam composition depends on the formulation of the composition. The stability of a foam may be determined by the time it takes for a foam to collapse, e.g., to 50% of its starting height. In some embodiments the foam composition has a collapse time of at least about 90 seconds. In some embodiments, the foam composition has a collapse time of at least about 120 seconds. In some embodiments, the foam composition has a collapse time of at least about 150 seconds. In some embodiments, the foam composition has a collapse time of at least about 180 seconds. In some embodiments, the foam composition has a collapse time of at least about 240 seconds. In some embodiments, the foam composition has a collapse time of at least about 300 seconds. In some embodiments, the foam composition has a collapse time of at least about 100 seconds, at least about 110 seconds, at least about 130 seconds, at least about 140 seconds, at least about 160 seconds, at least about 170 seconds, at least about 190 seconds, at least about 200 seconds, at least about 210 seconds, at least about 220 seconds, at least about 230 seconds, at least about 250 seconds, at least about 260 seconds, at least about 270 seconds, at least about 280 seconds, or at least about 290 seconds.

Over time, liquids and gas within a foam may separate, and the liquid may flow through the foam as a result of gravity. The “drainage” of the foam thus could be a measure of the stability of the foam. In some embodiments, the drainage time is the time it takes for the foam to degrade in quality, as defined by some measure, e.g., criteria as described herein. In some embodiments, a slow drainage is a drainage of about or greater than 180 sec. In some embodiments, foam drainage is at least about (i.e about or more than) 90 seconds, at least about 120 seconds, at least about 150 seconds, at least about 240 seconds, or at least about 300 seconds.

### Shakability

When the foamable composition has been packaged in an aerosol can or canister, it may be beneficial for a user to shake the canister before actuating the valve and releasing the foam. Shaking the canister before use homogenizes and disperses the propellant and foamable compositions within. To this end, it is also desirable for a user to be able to feel or hear the presence of the contents when the filled pressurized canister is shaken. As used herein, “shakability” refers to the degree to which the user is able to feel or hear the presence of the foamable composition when the filled pressurized canister is shaken. Shaking is done with mild to normal force without vigorous or excessive force. When the user cannot sense the motion of the contents during shaking the foamable composition may be considered to be non-shakable. When the user can moderately sense the motion of the contents during the shaking, the foamable composition is considered moderately shakable. When the contents are flowable during shaking, the product is considered shakable.

In one or more embodiments as described herein there is provided a composition and/or a foamable composition with improved fluidity. This in turn can lead to an improved shakability. In some embodiments, the foamable composition has improved shakability. In some embodiments, the foamable composition comprising hydrogenated castor oil has improved shakability, compared to foamable compositions not comprising hydrogenated castor oil. In some embodiments, the foamable composition comprising hydrogenated castor oil has similar shakability, on Day 0 and Day 15 at all temperatures tested, compared to foamable compositions comprising paraffin wax. In some embodiments, the foamable composition comprising hydrogenated castor oil has improved shakability, compared to foamable compositions comprising a paraffin wax. In some embodiments, the foamable composition comprising a paraffin wax has improved shakability compared to foamable compositions comprising hydrogenated castor oil. In some embodiments, the foamable composition comprising a wax has improved shakability compared to foamable compositions that do not comprise a wax. In some other embodiments, the foamable composition comprising a wax e.g., hydrogenated castor oil has reduced shakability compared to foamable compositions that do not comprise a wax. In some embodiments, the foamable composition is shakable for at least 1 day, at least 14 days, at least 15 days, at least 30 days, at least 60 days, at least 90 days, or at least 180 days when stored at 5° C. In some embodiments, the foamable composition is shakable for at least 12 months, or at least 18 months, or at least 24 months, or at least 30 months, or at least 36 months when stored at 5° C. In some embodiments, the foamable composition is shakable for at least 1 day, at least 14 days, at least 15 days, at least 30 days, at least 60 days, at least 90 days, or at least 180 days when stored at 25° C. In some embodiments, the foamable composition is shakable for at least 10 months, or at least 12 months, or at least 18 months, or at least 24 months when stored at 25° C. In some embodiments, the foamable composition is shakable for at least 1 day, at least 14 days, at least 15 days, at least 30 days, at least 60 days, at least 90 days, or at least 180 days when stored at 40° C. In some embodiments, the foamable composition is moderately shakable for at least 12 months, or at least 18 months, or at least 24 months, or at least 30 months, or at least 36 months when stored at 5° C. In some embodiments, the foamable composition is moderately shakable for at least 12 months, or at least 18 months, or at least 24 months, or at least 30 months, or at least 36 months when stored at 25° C. In some embodiments, the foamable composition is moderately shakable for at least 12 months, or at least 18 months, or at least 24 months, or at least 30 months, or at least 36 months when stored at 40° C.

### Flowability

The ability of the foamable composition to flow may also affect the passage of the composition upon release from the aerosol container. Depending on its flowability, a foamable composition can block a valve or a nozzle of the aerosol container, which is undesirable. In one or more embodiments, the foamable composition is flowable and does not block a valve of the aerosol container. In some embodiments, the foamable composition does not block a valve of the aerosol container for at least 1 day, or at least 10 days, or at least 15 days, or at least 30 days, or at least 60 days, or at least 90 days, or at least 180 days at room temperature. In some embodiments, the formulation is flowable at room temperature for at least about 3 months, at least about 6 months, at least about 9 months, at least about 12 months, at least about 8 months, or at least about 24 months.

In some embodiments, a non-shakable formulation has some flowability and allows formation of a foam of quality (e.g. good quality).

In some embodiment, a foamable composition described herein is provided in an aerosol or canister that comprises a nozzle that directs the composition from inside to outside of the canister. In some embodiment, a foamable composition described herein does not clog the nozzle or valve of the canister. In one or more embodiments, a foamable composition produced by a holding process does not result in clogging of the nozzle of the canister. In some embodiments, a foamable composition produced by a holding process results in substantially smaller degree of clogging of the nozzle of the canister as compared to a formulation prepared by a continuous heating-cooling process. In some embodiments, a foamable composition produced by a holding process results in less frequent clogging of the nozzle of the canister as compared to a formulation prepared by a continuous heating-cooling process. In one or more embodiments, a combination formulation comprising 2% HCO kept at 25° C. for 30 days is non-shakable when prepared by a continuous heating-cooling process and fully shakable when prepared by a holding process. In one or more embodiments, a combination formulation comprising 1.2% HCO kept at 25° C. for 30 days is moderately shakable when prepared by a continuous heating-cooling process and fully shakable when prepared by a holding process. In one or more embodiments, formulations kept at 40° C. for 30 days are non-shakable when prepared by a continuous heating-cooling process and moderately shakable when prepared by a holding process.

Formulations prepared by a holding process that have the property of superior shakability may comprise Tmh crystals, e.g., nonuniform, larger and more stable crystals that are not present in formulations prepared by a continuous heating-cooling process. Without being bound by any theory, the stable structures and crystal fingerprint formed by the holding process may be less available to interact with each other or with other components of the formulation and surprisingly improve the flowability of the formulation, resulting in a fully shakable formulation.

In some embodiments, preparation by a holding process does not change crystal polymorph, crystal configuration, or crystal lattice of the crystal in the formulation, but changes the formulation shakability.

In one or more embodiments, a formulation comprising a combination of MCH and ADP with 2% HCO prepared by a continuous heating-cooling process is not shakable after one to six months. In one or more embodiments, a formulation comprising MCH and ADP with 1.2% HCO prepared by a continuous heating-cooling process shows a moderate improvement in shakability as compared to a formulation with 2% HCO. In one or more embodiments, a formulation containing MCH and ADP with 1.2% HCO prepared by a holding process (e.g. 4 hours at about 54° C.) shows a significant improvement in shakability and is shakable at all timepoints tested at 25° C.

### Rheology

The flow point of a formulation may be measured by evaluated shear storage modulus (G′) or elastic modulus of a material, a measure of its ability to store deformation energy. In some embodiments, the foamable composition prepared using the methods disclosed herein has a G′ value between about 50 Pa and about 100 Pa after 1 day at 25° C. In some embodiments, the foamable composition has a G′ value between about 100 Pa and about 200 Pa after 1 day at 25° C. In some embodiments, the foamable composition has a G′ value between about 100 Pa and about 150 Pa after 1 day at 25° C. In some embodiments, the foamable composition has a G′ value between about 50 Pa and about 100 Pa after 15 days at 25° C. In some embodiments, the foamable composition has a G′ value between about 100 Pa and about 200 Pa after 15 days at 25° C. In some embodiments, the foamable composition has a G′ value between about 100 Pa and about 150 Pa after 15 days at 25° C. In some embodiments, the foamable composition has a G′ value between about 200 Pa and about 300 Pa after 15 days at 25° C. In some embodiments, the foamable composition has a G′ value between about 250 Pa and about 300 Pa after 15 days at 25° C. In some embodiments, the foamable composition has a G′ value between about 200 Pa and about 250 Pa after 15 days at 25° C. In some embodiments, the composition has a G′ value between about 200 Pa and about 300 Pa after 30 days at 25° C. In some embodiments, the composition has a G′ value between about 300 Pa and about 400 Pa after 30 days at 25° C. In some embodiments, the composition has a G′ value between about 400 Pa and about 500 Pa after 30 days at 25° C. In some embodiments, the composition has a G′ value between about 500 Pa and about 600 Pa after 30 days at 25° C. In some embodiments, the composition has a G′ value between about 500 Pa and about 550 Pa after 30 days at 25° C. In some embodiments, the composition has a G′ value between about 550 Pa and about 600 Pa after 30 days at 25° C. In some embodiments, the composition has a G′ value between about 4000 Pa and about 5000 Pa after 1 day at 40° C. In some embodiments, the composition has a G′ value between about 4000 Pa and about 4500 Pa after 1 day at 40° C. In some embodiments, the composition has a G′ value between about 4500 Pa and about 5000 Pa after 1 day at 40° C. In some embodiments, the composition has a G′ value between about 5000 Pa and about 6000 Pa after 1 day at 40° C. In some embodiments, the composition has a G′ value between about 6000 Pa and about 7000 Pa after 1 day at 40° C. In some embodiments, the composition has a G′ value between about 7000 Pa and about 8000 Pa after 1 day at 40° C. In some embodiments, the composition has a G′ value between about 8000 Pa and about 9000 Pa after 1 day at 40° C. In some embodiments, the composition has a G′ value between about 9000 Pa and about 10000 Pa after 1 day at 40° C. In some embodiments, the composition has a G′ value between about 6000 Pa and about 7000 Pa after 15 days at 40° C. In some embodiments, the composition has a G′ value between about 7000 Pa and about 8000 Pa after 15 days at 40° C. In some embodiments, the composition has a G′ value between about 8000 Pa and about 9000 Pa after 15 days at 40° C. In some embodiments, the composition has a G′ value between about 9000 Pa and about 10000 Pa after 15 days at 40° C. In some embodiments, the composition has a G′ value between about 10000 Pa and about 11000 Pa after 15 days at 40° C. In some embodiments, the composition has a G′ value between about 11000 Pa and about 12000 Pa after 15 days at 40° C. In some embodiments, the composition has a G′ value between about 12000 Pa and about 13000 Pa after 15 days at 40° C. In some embodiments, the composition has a G′ value between about 13000 Pa and about 14000 Pa after 15 days at 40° C. In some embodiments, the composition has a G′ value between about 14000 Pa and about 15000 Pa after 15 days at 40° C. In some embodiments, the composition has a G′ value between about 15000 Pa and about 16000 Pa after 15 days at 40° C. In some embodiments, the composition has a G′ value between about 16000 Pa and about 17000 Pa after 15 days at 40° C. In some embodiments, the composition has a G′ value between about 17000 Pa and about 18000 Pa after 15 days at 40° C. In some embodiments, the composition has a G′ value between about 18000 Pa and about 19000 Pa after 15 days at 40° C. In some embodiments, the composition has a G′ value between about 5000 Pa and about 6000 Pa after 30 days at 40° C. In some embodiments, the composition has a G′ value between about 6000 Pa and about 7000 Pa after 30 days at 40° C. In some embodiments, the composition has a G′ value between about 7000 Pa and about 8000 Pa after 30 days at 40° C. In some embodiments, the composition has a G′ value between about 8000 Pa and about 9000 Pa after 30 days at 40° C. In some embodiments, the composition has a G′ value between about 9000 Pa and about 10000 Pa after 30 days at 40° C. In some embodiments, the composition has a G′ value between about 10000 Pa and about 11000 Pa after 30 days at 40° C. In some embodiments, the composition has a G′ value between about 11000 Pa and about 12000 Pa after 30 days at 40° C. In some embodiments, the composition has a G′ value between about 12000 Pa and about 13000 Pa after 30 days at 40° C. In some embodiments, the composition has a G′ value between about 13000 Pa and about 14000 Pa after 30 days at 40° C. In some embodiments, the composition has a G′ value between about 14000 Pa and about 15000 Pa after 30 days at 40° C. In some embodiments, the composition has a G′ value between about 15000 Pa and about 16000 Pa after 30 days at 40° C. In some embodiments, the composition has a G′ value between about 16000 Pa and about 17000 Pa after 30 days at 40° C. In some embodiments, the composition has a G′ value between about 17000 Pa and about 18000 Pa after 30 days at 40° C. In some embodiments, the composition has a G′ value between about 18000 Pa and about 19000 Pa after 30 days at 40° C.

Topical therapeutic breakable gel and foamable compositions comprising tetracycline, including those without surfactants, have been described, for example in U.S. application Ser. Nos. 13/499,501, 13/499,727, 13/499,475, and 13/499,709, U.S. Publication No. 2011/0281827, WO 11/039637, WO 11/039638, WO 11/138678 and WO 2011/064631, all of which are herein incorporated in their entirety by reference. More particularly, any of the active ingredients, carriers, solvents, surfactants, foam adjuvants, fatty acids, fatty alcohols, polymeric agents, penetration enhancers, preservatives, humectants, moisturizers, and other excipients, as well as the propellants and methods listed therein can be applied herein and are incorporated by reference.

Other carriers and compositions are described in: U.S. Publication No. 2005/0232869, published on Oct. 20, 2005, entitled NONSTEROIDAL IMMUNOMODULATING KIT AND COMPOSITION AND USES THEREOF; U.S. Publication No. 2005/0205086, published on Sep. 22, 2005, entitled RETINOID IMMUNOMODULATING KIT AND COMPOSITION AND USES THEREOF; U.S. Publication No. 2006/0018937, published on Jan. 26, 2006, entitled STEROID KIT AND FOAMABLE COMPOSITION AND USES THEREOF; U.S. Publication No. 2005/0271596, published on Dec. 8, 2005, entitled VASOACTIVE KIT AND COMPOSITION AND USES THEREOF; U.S. Publication No. 2006/0269485, published on Nov. 30, 2006, entitled ANTIBIOTIC KIT AND COMPOSITION AND USES THEREOF; U.S. Publication No. 2007/0292355, published on Dec. 20, 2007, entitled ANTI-INFECTION AUGMENTATION OF FOAMABLE COMPOSITIONS AND KIT AND USES THEREOF; U.S. Publication No. 2008/0317679, published on Dec. 25, 2008, entitled FOAMABLE COMPOSITIONS AND KITS COMPRISING ONE OR MORE OF A CHANNEL AGENT, A CHOLINERGIC AGENT, A NITRIC OXIDE DONOR, AND RELATED AGENTS AND THEIR USES; U.S. Publication No. 2008/0044444, published on Feb. 21, 2008, entitled DICARBOXYLIC ACID FOAMABLE VEHICLE AND PHARMACEUTICAL COMPOSITIONS THEREOF; U.S. Publication No. 2008/0069779, published on Mar. 20, 2008, entitled FOAMABLE VEHICLE AND VITAMIN AND FLAVONOID PHARMACEUTICAL COMPOSITIONS THEREOF; U.S. Publication No. 2008/0206159, published on Aug. 28, 2008, entitled COMPOSITIONS WITH MODULATING AGENTS; U.S. Publication No. 2008/0206161, published on Aug. 28, 2008, entitled QUIESCENT FOAMABLE COMPOSITIONS, STEROIDS, KITS AND USES THEREOF; U.S. Publication No. 2008/0260655, published on Oct. 23, 2008, entitled SUBSTANTIALLY NON-AQUEOUS FOAMABLE PETROLATUM BASED PHARMACEUTICAL AND COSMETIC COMPOSITIONS AND THEIR USES; U.S. Publication No. 2011/0268665, published on Nov. 3, 2011, entitled OIL-BASED FOAMABLE CARRIERS AND FORMULATIONS; U.S. Publication No. 2012/0087872, published on Apr. 12, 2012, entitled FOAMABLE VEHICLES AND PHARMACEUTICAL COMPOSITIONS COMPRISING APROTIC POLAR SOLVENTS AND USES THEREOF; U.S. Publication No. 2012/0213709, published on Aug. 23, 2012, entitled NON SURFACTANT HYDRO-ALCOHOLIC FOAMABLE COMPOSITIONS, BREAKABLE FOAMS AND THEIR USES; U.S. Publication No. 2012/0213710, published on Aug. 23, 2012, entitled SURFACE ACTIVE AGENT NON POLYMERIC AGENT HYDRO-ALCOHOLIC FOAMABLE COMPOSITIONS, BREAKABLE FOAMS AND THEIR USES; U.S. Publication No. 2013/0064777, published on Mar. 14, 2013, entitled SURFACTANT-FREE WATER-FREE FOAMABLE COMPOSITIONS, BREAKABLE FOAMS AND GELS AND THEIR USES; U.S. Publication No. 2013/0053353, published on Feb. 28, 2013, entitled COMPOSITIONS, GELS AND FOAMS WITH RHEOLOGY MODULATORS AND USES THEREOF; U.S. Publication No. 2011/0281827, published on Nov. 17, 2011, entitled COMPOSITIONS, GELS AND FOAMS WITH RHEOLOGY MODULATORS AND USES THEREOF; U.S. Publication No. 2013/0028850, published on Jan. 31, 2013, entitled TOPICAL TETRACYCLINE COMPOSITIONS; U.S. Publication No. 2013/0011342, published on Jan. 10, 2013, entitled SURFACTANT-FREE, WATER-FREE, FOAMABLE COMPOSITIONS AND BREAKABLE FOAMS AND THEIR USES; U.S. Publication No. 2013/0225536, published on Aug. 29, 2013, entitled COMPOSITIONS FOR THE IMPROVED TREATMENT OF ACNE AND RELATED DISORDERS; U.S. Publication No. 2014/0121188, published on May 1, 2014, entitled METHODS FOR ACCELERATED RETURN OF SKIN INTEGRITY AND FOR THE TREATMENT OF IMPETIGO; U.S. Publication No. 2015/0164922, published on Jun. 18, 2015, entitled USE OF TETRACYCLINE COMPOSITIONS FOR WOUND TREATMENT AND SKIN RESTORATION, Compositions and Methods for Treating Rosacea and Acne U.S. Publication No. 2018/0064638, oil foamable carriers and formulations, U.S. Publication No. US 2019/0091149 all of which are incorporated herein by reference in their entirety. More particularly, any of the active ingredients, carriers, solvents, surfactants, foam adjuvants, polymeric agents, penetration enhancers, preservatives, humectants, moisturizers, and other excipients, as well as the propellants and methods listed therein can be applied herein and are incorporated by reference.

### Administration

In one or more embodiments there is provided a method of administering a foamable composition to a target area such as skin of a patient comprising releasing foam, applying it to the area, and collapsing the foam. In one or embodiments, the foam is applied by spreading. In the course of spreading, mechanical shear can cause the foam to collapse. In one or more embodiments, the collapsed foam is not washed off. In one or more embodiments it is absorbed onto the area of skin. In one or more embodiments it avoids skin irritation or an ointment sensation.

In one or more embodiments, there is provided a method for reducing the number of acne or rosacea lesions, by applying topically an effective amount of a foam or foamable composition to an afflicted area of a patient in need. In one or more embodiments, the method involves applying a foam or foamable composition topically to a target surface in need of treatment and breaking the foam over the target site. In one or more embodiments the foam or foamable composition is collapsed and spread by application of a mechanical force, which can be mild or slight such as a simple rub and the active agent is then absorbed. In one or more embodiments the foam or foamable composition is absorbed.

In one or more embodiments, a foam or foamable composition is absorbed within 240 seconds, or within 200 seconds, or within 180 seconds, or within 150 seconds, within 120 seconds, or within 100 seconds, or within 80 seconds, or within 60 seconds, or within 50 seconds, or within 40 seconds, or within 30 seconds, or within 20 seconds, or within 10 seconds, or within 5 seconds, or within 2 seconds or less. The term “absorbed” means that the composition enters onto and into an area of skin, mucosa or eye, often forming a thin coating on the surface.

In one or more embodiments, the method uses an additional step of pre cleaning and drying the lesions and surrounding area before applying the foam or foamable composition.

In one or more embodiments, the method uses a sterile applicator or prior to the steps of administering and/or collapsing and/or spreading, the hands of the person spreading are sterilized in order to avoid cross contamination.

In one or more other embodiments, the method comprises an additional step of applying an active agent to the lesions and surrounding area after the foam or foamable composition has been absorbed, wherein the active agent is a hyaluronic acid or a retinoid or benzyl peroxide (“BPO”) or salicylic acid, or an alpha hydroxy acid, or azelaic acid, or nicotinamide, or a keratolytic agent, or clindamycin, or metronidazole, or doxycycline, or erythromycin, or ivermectin, or brimonidine, or sodium sulfacetamide and sulfur, or tretinoin. In some embodiments, the active agent, such as, for example, a hyaluronic acid, a retinoid, BPO, salicylic acid, an alpha hydroxy acid, azelaic acid, a nicotinamide, a keratolytic agent, clindamycin, metronidazole, erythromycin, ivermectin, brimonidine, sodium sulfacetamide and sulfur, tretinoin, or mixtures of two or more thereof, is applied once daily at least 1, 2, 3, 4, 5, 6, 7, 8, 9, 10, 11, or 12 hours after the tetracycline antibiotic formulation has been absorbed. In further embodiments, the active agent, such as, for example, a hyaluronic acid or a retinoid or BPO or salicylic acid, or an alpha hydroxy acid, or azelaic acid, or nicotinamide, or a keratolytic, or clindamycin, or metronidazole, or erythromycin, or ivermectin, or brimonidine, or sodium sulfacetamide and sulfur, or tretinoin, is applied after the third day. In yet additional embodiments, the active agent, such as, for example, a hyaluronic acid or a retinoid or BPO or salicylic acid, or an alpha hydroxy acid, or azelaic acid, or nicotinamide, or a keratolytic agent, or clindamycin, or metronidazole, or erythromycin, or ivermectin, or brimonidine, or sodium sulfacetamide and sulfur, or tretinoin, is applied during the maintenance stage. In an alternative embodiment, the active agent, such as, for example, a hyaluronic acid or a retinoid or BPO or salicylic acid, or an alpha hydroxy acid, or azelaic acid, or nicotinamide, or a keratolytic agent, or clindamycin, or metronidazole, or erythromycin, or ivermectin, or brimonidine, or sodium sulfacetamide and sulfur, or tretinoin, is replaced with or supplemented by a steroid.

In an alternative embodiment, the active agent, such as, for example, a hyaluronic acid or a retinoid or BPO or salicylic acid, or an alpha hydroxy acid, or azelaic acid, or nicotinamide, or a keratolytic agent or steroid, or clindamycin, or metronidazole, or erythromycin, or ivermectin, or brimonidine, or sodium sulfacetamide and sulfur, or tretinoin, is replaced with or supplemented by an antibiotic. In an embodiment, the antibiotic, which is in addition to one or more tetracycline antibiotics, is selected from the group consisting of mupirocin, fusidic acid, a penicillin or penicillin derivative, augmentin, an antistaphylococcal penicillin, amoxicillin/clavulanate, a cephalosporin, cephalexin, a macrolide, erythromycin, clindamycin, trimethoprim-sulfamethoxazole penicillin, retapamulin, and mixtures of any two or more thereof. In some embodiments, the antibiotic is applied topically. In some embodiments, the antibiotic is applied orally, by injection, or by infusion. In some embodiments, more than one antibiotic is applied. For example, an antibiotic is applied topically and another is given orally. This scenario can be appropriate in some instances, e.g., where there is systemic as well as topical bacterial infection.

### Frequency

In some embodiments there is provided a regime or regimen for treating a patient having acne, and/or acne related symptoms, using the compositions and/or foamable compositions described herein. In some embodiments, the regimen comprises administering topically the foams or foamable compositions or compositions described herein once, twice, three times, four times, five times, or six times a day to a surface having acne. In some embodiments, the regimen comprises administering topically the foam, foamable composition, or compositions described herein once every day, every two days, every three days, every four days, every five days, every six days, or every seven days to a surface having acne. In some embodiments, the regimen comprises administering topically the foam, foamable composition, or compositions described herein intermittently, or as needed, to a surface having acne. In some embodiments, the regimen comprises administering the foam, foamable composition, or compositions described herein to a surface having acne, for at least one week, at least two weeks, at least three weeks, at least four weeks, at least five weeks, at least six weeks, at least seven weeks, at least eight weeks, at least three months, at least four months, at least five months, at least six months, at least seven months, at least eight months, at least night months, at least ten months, at least eleven months, at least one year, at least two years, at least three years, at least four years at least five years, at least six years, at least seven years, at least eight years, at least nine years or at least ten years.

### Methods of Treatment

In some embodiments, foam, foamable composition, or compositions e.g., foamable compositions and or compositions comprising tetracycline-based antibiotics disclosed herein, can be administered topically to treat skin disorders, e.g., acne conglobata, acne vulgaris, rosacea. The terms “treat,” “treatment,” and other related forms of the term comprise a step of administering, e.g., topically, an effective dose, or effective multiple doses, of a foamable composition and/or composition comprising a tetracycline-based antibiotic as disclosed herein to an animal (including a human being) in need thereof. If the dose is administered prior to onset of symptoms of a disorder/disease, the administration is prophylactic. If the dose is administered after the development of a disorder/disease, the administration is therapeutic. In embodiments, an effective dose is a dose that partially or fully alleviates (i.e., eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Examples of disease states contemplated for treatment are set out herein.

### Acne

“Acne” is a general term that describes a common skin disorder, which afflicts many people. The prevalence of adult acne is about 3% in men and between about 11% and 12% in women. Moderate to severe acne is observed in 14% of acne patients. There are various types of acne recognized in the field, including, for example: acne vulgaris, acne conglobate, acne fulminans, and nodular papulopustular acne. Acne vulgaris (cystic acne or simply acne) is generally characterized by areas of skin with seborrhea (scaly red skin), comedones (blackheads and whiteheads), papules (pinheads), pustules (pimples), nodules (large papules) and/or possibly scarring. Acne can be inflammatory acne and or non-inflammatory acne. Acne vulgaris may affect the face, the upper part of the chest, and the back. Severe acne vulgaris is inflammatory, but acne vulgaris can also manifest in non-inflammatory forms. Acne conglobata is a severe form of acne, and may involve many inflamed nodules that are connected under the skin to other nodules. Acne conglobata often affects the neck, chest, arms, and buttocks.

In various embodiments, patients treated with the compositions disclosed herein are first diagnosed with acne. For example, patients may be diagnosed according to Eichenfield et al, using a differential diagnosis for pediatric acne which depends on, inter alia, age group, pubertal status, and form of presentation. For instance, for adolescents (˜12-18 y of age), differential diagnosis may turn on, inter alia, detection of one or more of demodex folliculitis, gram-negative folliculitis, keratosis pilaris, malassezia (pityrosporum) folliculitis, popular sarcoidosis, perioral dermatitis, pseudofolliculitis barbae, and tinea faciei. For preadolescent (≥7 to ≤12 y of age), acne may present as acne venenata or pomade acne (from the use of topical oil-based products), and diagnosis may turn on, inter alia, detection of one or more of angiofibromas or adenoma sebaceum, corticosteroid-induced acne, flat warts, keratosis pilaris, milia, molluscum contagiosum, perioral dermatitis, and syringomas. Mid-childhood acne (1-7 y of age) may warrant an endocrinologic workup for causes of hyperandrogenism, and may be associated with adrenal tumors, congenital adrenal hyperplasia, cushing syndrome, gonadal tumors, ovarian tumors, PCOS, premature adrenarche, or true precocious puberty. In addition to differential diagnosis, Eichenfield et al teaches that history and physical examination are also useful for diagnosis in pediatric acne. The physical examination may include type and distribution of acne lesions, height, weight, growth curve, and possible blood pressure abnormalities. The assessment for preadolescent acne may also include history and physical examination, with further workup if there are signs of excess androgens PCOS, or other systemic abnormalities. Preadolescent acne may be characterized by a predominance of comedones on the forehead and central face (the so-called “T-zone”) with relatively few inflammatory lesions. Early presentation may include comedones of the ear. Mid-childhood acne typically presents primarily on the face with a mixture of comedones and inflammatory lesions. See, Eichenfield et al., Pediatrics. (2013); 131 (suppl 3): S163-S186. Zaenglein et al describes other acne diagnostic tools that may be used, e.g., in adults treated with a composition disclosed herein, taking into account various factors, such as type of acne, severity of acne, number of acne lesions, anatomic location/extent of acne, quality of life and other psychosocial metrics, and scarring. Scientific measures, such as ultraviolet-induced red fluorescence, casual sebum level, skin capacitance imaging, skin surface pH, and transepidermal water loss may help to more objectively classify and rate acne. See, Zaenglein J AM ACAD DERMATOL (MAY 2016), 949-950.

In some embodiments, provided herein is a foamable combination product (3% minocycline/0.3% adapalene as active agents) for treating acne. In a further embodiment, the acne is moderate-to-severe acne vulgaris. In one or more embodiments the combination product is more effective than the vehicle without the active agents. In one or more embodiments the combination product is more effective than the product with minocycline alone. In one or more embodiments the combination product is more effective than the product with adapalene alone. In one or more embodiments the product is delivered as a foam.

In some embodiments, provided is a method for treating acne conglobata or acne vulgaris in a patient in need thereof, the method comprising: administering to the patient a composition and/or foamable composition or foam as described herein, wherein the foamable composition or foam comprises a tetracycline antibiotic. In some embodiments, compositions and/or foamable compositions or foams comprising 3% minocycline and 0.3% adapalene are administered topically once daily to the full face and other acne-affected areas of the body for 12 weeks. In some embodiments, compositions and/or foamable compositions or foams comprising 3% minocycline are administered topically once daily to the full face and other acne-affected areas of the body for 12 weeks. In some embodiments, compositions and/or foamable compositions or foams comprising 0.3% adapalene are administered topically once daily to the full face and other acne-affected areas of the body for 12 weeks. In some embodiments, the compositions and/or foamable compositions or foams are applied daily at a fixed time, e.g., about 1 hour before bedtime or about 2 hours before bedtime, to the full face and other acne-affected areas of the body.

In some embodiments, the daily dose is no more than 150 mg of minocycline, e.g., about 140 mg, about 130 mg, about 120 mg, or about 110 mg. In some embodiments, the daily dose is no more than 15 mg adapalene, e.g., about 14 mg, about 13 mg, about 12 mg, about 11 mg, about 10 mg, or about 9 mg. In some embodiments, the daily dose is about 106 mg of minocycline. In some embodiments, the daily dose is about 10.6 mg adapalene. In some embodiments, the cumulative maximal dose is no more than 12 g of minocycline, e.g., 11 g, 10 g, 9 g, 8 g, or 7 g. In some embodiments, the cumulative maximal dose is no more than 8.9 g of minocycline. In some embodiments, the cumulative maximal dose is no more than 1.2 g of adapalene, e.g., 1.1 g, 1.0 g, 0.9 g, 0.8 g, or 0.7 g. In some embodiments, the cumulative maximal dose is no more than 0.89 g of adapalene.

In some embodiments, the patient is 8-100 years old. In some other embodiments, the patient is at least 12 years of age. In some embodiments, the patient is a male patient, e.g., one who is 8-100 years old, or at least 12 years old. In some embodiments, the patient is a female patient, e.g., one who is 8-100 years old, or at least 12 years old.

In some embodiments, the patient has been or concurrently is diagnosed with acne conglobata or acne vulgaris prior to treatment, e.g., by a clinical examination. In some embodiments, acne conglobata or acne vulgaris is diagnosed by a clinical evaluation of symptoms, e.g. Investigator's Global Assessment (IGA) of Acne Severity. In some embodiments, acne conglobata or acne vulgaris is defined as any disorder of the skin whose initial pathology is microscopic microcomedo. In some embodiments, microcomedo evolve into visible open comedones (“blackheads”) or closed comedones (“whiteheads”). In some embodiments, patients are diagnosed with acne conglobata or acne vulgaris based on a physical examination.

The severity of the disease may be determined by lesion counting. In some embodiments, microcomedo evolve into inflammatory papules, pustules, and nodules. In some embodiments, the severity of acne conglobata or acne vulgaris is determined by lesion counting. In some embodiments, the severity of acne conglobata or acne vulgaris is determined by counting the number of open comedones on the affected area. In some embodiments, the severity of acne conglobata or acne vulgaris is determined by counting the number of closed comedones on the affected area. In some embodiments, the severity of acne conglobata or acne vulgaris is determined by counting the number of papules in the affected area. In some embodiments, the severity of acne conglobata or acne vulgaris is determined by counting the number of pustules in the affected area. In some embodiments, the severity of acne conglobata or acne vulgaris is determined by counting the number of nodules in the affected area. In some embodiments, the severity of acne conglobata or acne vulgaris is determined by counting the number of papules and pustules in the affected area. In some embodiments, the severity of acne conglobata or acne vulgaris is determined by counting the number of papules, pustules, and nodules in the affected area. In some embodiments, the patient has 10-100 inflammatory lesions (papules and/or pustules) on the face. In some embodiments, there are 20-50 inflammatory lesions (papules and/or pustules) on the face. In some embodiments, there are 0-200 non-inflammatory lesions (open and closed comedones) on the face. In some embodiments, there are 20-150 non-inflammatory lesions (open and closed comedones) on the face. In some embodiments, there are 25-100 non-inflammatory lesions (open and closed comedones) on the face.

The Investigator's Global Assessment (IGA) of Acne Severity is a classification scheme for the severity of primary acne vulgaris on a scale of 0 to 4. Pochi et al., J. Am. Acad. Dermatol., (1991) 24:495-500. A score of zero indicates that the acne is ‘clear’ and residual hyperpigmentation and erythema may be present. A score of one indicates that the acne is “almost clear” with a few scattered comedones and a few small papules. A score of two indicates that the acne is “mild” with some comedones and some papules and pustules, and less than half the face is involved. A score of three indicates that the acne is ‘moderate’ with more than half the face affected, many comedones, papules, and pustules, a possibly one nodule present. A score of four indicates that the acne is ‘severe’ with involvement of the entire face, numerous papules and pustules, and a few nodules and cysts. In some embodiments, the patient has a IGA score of at least 2, e.g., 2, 3, or 4. In some embodiments, a patient treated with a composition disclosed herein has an IGA score of 3 or 4. In some embodiments, the patient has no more than one active nodule on the face. In some embodiments, the patient has no more than two active nodules on the face.

In some embodiments, the patient is diagnosed with acne vulgaris, with at least one or more of (1) 20 to 50 inflammatory lesions (papules and/or pustules) on the face; (2) 25 to 100 non-inflammatory lesions (open and closed comedones) on the face; and (3) IGA score of moderate (3) to severe (4).

The pre-screening of patients amenable to treatment is also contemplated, e.g., according to the methods of identifying acne vulgaris patients disclosed herein, as well as the administration of treatment to patients identified according to criteria disclosed herein. In some embodiments, a female patient does not have a positive urine pregnancy test. In some embodiments, the patient uses an effective method of contraception while undergoing treatment. In some embodiments, the patient using a hormonal contraceptive uses the same type and strength of contraceptive over the 3 months prior to the start of treatment. In some embodiments, the patient does not use any other acne medication concurrently with the treatment described herein. In some embodiments, the patient does not use a medicated cleanser during the treatment described herein. In some embodiments, the patient does not have excessive sun exposure during the treatment described herein. In some embodiments, the patient does not use a tanning booth during the treatment described herein.

Methods of selecting patients who will benefit from the treatment disclosed here are also contemplated herein. In some embodiments, a patient who is female is not pregnant or lactating. In some embodiments, the patient does not have acne conglobata, acne fulminans, secondary acne (chloracne, drug-induced acne), or any dermatological condition of the face that could interfere with the clinical evaluations. In some embodiments, the patient does not have facial hair, e.g., beard or mustache, that could interfere with clinical evaluations. In some embodiments, the patient does not have a sunburn on the face. In some embodiments, the patient does not have any sever systemic disease, e.g., lupus, multiple schlerosis, that could interfere with clinical evaluations. In some embodiments, the patient does not have a documented history of an allergy to tetracycline-class antibiotics, or to any components in the pharmaceutical compositions described herein. In some embodiments, the patient does not have a documented history of pseudomembranous colitis or antibiotic-associated colitis. In some embodiments, the patient does not have a documented history of hepatitis or clinically significant liver damage or renal impairment. In some embodiments, the patient does not have a documented history of a known or suspected premalignant or malignant disease excluding successfully treated skin cancers.

In some embodiments, the patient has not used medicated facial cleansers within one week prior to the start of treatment. In some embodiments, the patient has not used a topical acne treatment on the face within one week prior to the start of treatment. In some embodiments, the patient has not used topical retinoids on the face within four weeks prior to the start of treatment. In some embodiments, the patient has not used topical anti-inflammatories and/or corticosteroids on the face within four weeks prior to the start of treatment. In some embodiments, the patient has not used topical corticosteroids on body areas other than the face for more than 15 consecutive days and on more than 10% of the body surface area within four weeks prior to the start of treatment. In some embodiments, the patient has not used topical corticosteroids on body folds, such as axillary and inguinal regions, for more than 15 consecutive days within four weeks prior to the start of treatment. In some embodiments, the patient has not used systemic antibiotics within four weeks prior to the start of treatment. In some embodiments, the patient has not used systemic acne treatments within four weeks prior to the start of treatment. In some embodiments, the patient has not used systemic retinoids within twelve weeks prior to the start of treatment. In some embodiments, the patient has not used systemic corticosteroids within twelve weeks prior to the start of treatment. In some embodiments, the patient has not used a sauna within two weeks prior to the start of treatment. In some embodiments, the patient has not undergone epilation of the face within two weeks prior to the start of treatment. In some embodiments, the patient does not have folliculitis on the face. In some embodiments, the patient does not have documented drug addiction or alcohol abuse within two years prior to the start of treatment, with heavy drinking levels defined by. The Substance Abuse and Mental Health Services Administration (SAMHSA) as drinking 5 or more alcoholic drinks on the same occasion on each of 5 or more days in the past 30 days. In some embodiments, the patient does not have a documented history of depression that is not adequately controlled by medication at the time of treatment.

Methods of assessing treatment efficacy and/or clinical improvement are described herein and known in the art. Any suitable method of assessment may be used. In some embodiments, treatment efficacy is assessed by comparing the patient to a control, e.g., a subject who does not have acne, historic data, and/or taking into account the developmental history of acne. In some embodiments, treatment efficacy is assessed by comparing the patient condition before and after receipt of treatment to the known natural history of acne. In some embodiments, treatment efficacy is assessed by comparing the patient's current condition with the patient's condition prior to treatment (baseline). In some embodiments, treatment efficacy is assessed by comparing the condition of one group of patients at different time points after receiving a composition comprising one active agent with that of a second group of patients receiving a composition comprising a second active agent. In some embodiments, treatment efficacy is assessed by comparing the condition of one group of patients at different time points after receiving composition comprising one active agent to that of a second group of patients after receiving a composition comprising a combination of active agents. In some embodiments, treatment efficacy is assessed by comparing the condition of one group of patients' after receiving the composition comprising one or more active agent with patients' condition receiving the vehicle at different time points compared to baseline. For example, treatment efficacy for a patient with acne vulgaris may be assessed by the following parameters (1) reduction in the number of lesions, (2) no increase in the number of lesions, or (3) fewer lesions as compared to a patient who has not undergone treatment. In some embodiments, treatment may be considered efficacious if symptoms stabilize and/or do not worsen, e.g., no further increase in the number of lesions or no further progression of symptoms if observed. In some embodiments, treatment may be considered efficacious if an improvement in symptoms is observed, e.g. decrease in the number of lesions or improvement in IGA score. In some embodiments, treatment may be considered efficacious if IGA Treatment Success, e.g where success is defined as an IGA score of 0 or 1, and at least a 2-grade improvement (decrease) from Baseline.

In some embodiments, treatment efficacy is assessed by the absolute change in inflammatory lesion count after the start of treatment, e.g., 4 weeks, 8 weeks, or 12 weeks after the start of treatment, compared to the baseline before treatment. In some embodiments, treatment efficacy is assessed by the absolute change in inflammatory lesion count 12 weeks after the start of treatment, compared to the baseline before treatment. In some embodiments, treatment efficacy is assessed by the absolute change in non-inflammatory lesion count after the start of treatment, e.g., 4 weeks, 8 weeks, or 12 weeks after the start of treatment, compared to the baseline before treatment. In some embodiments, treatment efficacy is assessed by the absolute change in non-inflammatory lesion count 12 weeks after the start of treatment, compared to the baseline before treatment. In some embodiments, treatment efficacy is assessed by a 2-grade decrease on the IGA scale after the start of treatment, e.g., 4 weeks, 8 weeks, or 12 weeks after, compared to the baseline before treatment. In some embodiments, treatment efficacy is assessed by a 2-grade decrease on the IGA scale 12 weeks after the start of treatment, compared to the baseline before treatment. In some embodiments, treatment efficacy is assessed by a score of 0 or 1 on the IGA scale after the start of treatment, e.g., 4 weeks, 8 weeks, or 12 weeks after, compared to the baseline before treatment. In some embodiments, treatment efficacy is assessed by a score of 0 or 1 on the IGA scale 12 weeks after the start of treatment, compared to the baseline before treatment.

In some embodiments, treatment efficacy is assessed by a score of 0 or 1 on the IGA scale and a 2-grade decrease on the IGA scale 12 weeks after the start of treatment, compared to the baseline before treatment. In some embodiments, treatment efficacy is assessed by a score of 0 or 1 on the IGA scale and a 2-grade decrease on the IGA scale after the start of treatment, e.g., 4 weeks, 8 weeks, or 12 weeks after, compared to the baseline before treatment. In some embodiments, treatment satisfaction is assessed by subject satisfaction questionnaire. In some embodiments, treatment satisfaction is assessed by subject satisfaction questionnaire 12 weeks after the start of treatment, compared to the baseline before treatment. In some embodiments, subject satisfaction is assessed by comparing the percent in condition improvement in one group of patients' after receiving the composition comprising one or more active agent or receiving the vehicle at different time points compared to baseline.

In some embodiment's safety is assessed using measures, including physical examinations, the monitoring of vital signs, TEAEs (volunteered, observed, and elicited by general questioning in a non-suggestive manner), and local skin tolerability assessments. Tolerability to a drug or treatment can be measured by the appearance of adverse side effects or toxicity. In some embodiments, tolerability to the treatment is assessed. In some embodiments, local skin tolerability (face only) is assessed based on subject-rated itching and stinging/burning, and assessments of dryness, scaling, erythema and hyperpigmentation. In some embodiments, treatment-emergent adverse events (TEAE) are assessed after the start of treatment, e.g., 4 weeks, 8 weeks, 12 weeks or 16 weeks after treatment. In some embodiments, TEAE are assessed by patient questionnaires, observations, physical examinations, vital signs and local skin tolerability assessments, which may include itching, stinging, burning, dryness, scaling, erythema, and hyperpigmentation. In some embodiments, TEAE or local skin tolerability are assessed by the same evaluator throughout the study whenever possible.

In one or more embodiments there is provided a method of treating a subject for acne, comprising diagnosing a subject having acne as at risk for tissue damage, wherein tissue damage comprises scarring, post inflammatory hyperpigmentation and/or residual erythema.

In one or more embodiments there is provided a method of treating a subject for acne with combination product (e.g. a composition comprising a combination of minocycline 3% and adapalene 0.3%, e.g., one prepared with or without a holding step) which is significantly superior to vehicle with respect to absolute change from baseline in inflammatory lesion counts and IGA treatment success after 12 weeks of treatment. In one or more embodiments there is provided a method of treating a subject for acne with combination product which is numerically superior to vehicle with respect to absolute change from baseline in non-inflammatory lesion counts. In one or more embodiments these findings are supported by sensitivity analyses and results of secondary efficacy analyses.

In one or more embodiments there is provided a method of treating a subject for acne with combination product wherein secondary efficacy analyses compare the combination product with the two individual active agents, minocycline 3% or adapalene 0.3%. In one or more embodiments there is provided a method of treating a subject for acne with combination product is statistically superior to adapalene 0.3% in absolute change from baseline in inflammatory and non-inflammatory lesion counts at week 12. In one or more embodiments there is provided a method of treating a subject for acne wherein treatment with combination product is statistically superior to minocycline 3% in absolute change from baseline in non-inflammatory lesion counts at week 12. In one or more embodiments there is provided a method of treating a subject for acne wherein treatment with combination product is numerically superior to minocycline 3% in absolute change from baseline in inflammatory lesion counts at week 12. In one or more embodiments there is provided a method of treating a subject for acne wherein treatment with combination product is statistically superior for IGA treatment success, to adapalene 0.3% at week 12. In one or more embodiments there is provided a method of treating a subject for acne, wherein treatment with combination product is numerically superior for IGA treatment success, to minocycline 3% at week 12.

Without being bond by any theory, the combination of minocycline and adapalene in the vehicle is effective against acne and all three components can contribute to successful treatment that is advantageous in treating both inflammatory and non-inflammatory lesions and can provide subjects with new and better treatment options.

In one or more embodiments there is provided a method of treating a subject for acne, wherein treatment with a combination product exhibits a favorable safety profile, with the majority of TEAEs being characterized as mild or moderate. In one or more embodiments, there is provided a method of treating a subject for acne, wherein the primary treatment-related TEAEs include dry skin, rash, acne, and eye irritation. In one or more embodiments, there is provided a method of treating a subject for acne with a combination product, wherein the potential for TEAEs is reduced.

In one or more embodiments there is provided a method of treating a subject for acne, wherein treatment with a combination product is effective and safe with administration of other concomitant medicines (e.g., sex hormones and modulators of the genital system, other gynecologicals, psychoanaleptics, vitamins, analgesics, anti-inflammatory and antirheumatic products, and/or antihistamines for systemic use, emollients and protectives) with no apparent drug interactions. In one or more embodiments there is provided a method of treating a subject for acne, wherein the most commonly used class of concomitant medications is sex hormones and modulators of the genital system.

In one or more embodiments there is provided a method of treating a subject for acne, wherein the number of subjects reporting concomitant medications is highest in the adapalene 0.3% arm. In one or more embodiments there is provided a method of treating a subject for acne, wherein the most commonly used class of concomitant medications in the adapalene 0.3% arm is sex hormones and modulators of the genital system.

### Rosacea

Rosacea is a chronic acneiform disorder affecting skin and potentially the eye. It is a syndrome of undetermined etiology characterized by both vascular and papulopustular components involving the face and occasionally the neck, scalp, ears and upper trunk. Clinical findings include mid facial erythema, telangiectasis, papules and pustules, and sebaceous gland hypertrophy. Rosacea is characterized by episodic flushing of affected areas, which can be triggered by various factors, such as consumption of alcohol, hot drinks, spicy foods or physical exercise. Facial rosacea is classified/graded in multiple clinical forms: (1) erythematotelangiectatic rosacea which is characterized by (semi-) permanent erythema and/or flushing; (2) papulopustular rosacea, characterized by presence of inflammatory lesions such as papules and pustules; (3) phymatous rosacea characterized by circumscribed permanent swelling/thickening of skin areas, typically the nose; and (4) ocular rosacea characterized by the appearance of redness in eyes and eyelids due to telangiectasias and inflammation, feeling of dryness, irritation, or gritty, foreign body sensations, itching, burning, stinging, and sensitivity to light, eyes being susceptible to infection, or blurry vision.

In various embodiments, a patient treated with a composition disclosed herein is first diagnosed with rosacea. The diagnosis of rosacea may be made clinically, e.g., based on visible assessment and patient history, after other causes of facial erythema and/or papulopustular skin lesions have been excluded, including contact dermatitis, seborrheic dermatitis, photodamage, acne vulgaris, cutaneous lupus, and carcinoid syndrome. For example, patients may be diagnosed according to the Standard Management Options for Rosacea: the 2019 Update by the National Rosacea Society Expert Committee. Diagnostic features of rosacea may include, inter alia, fixed centrofacial erythema in a characteristic pattern that may periodically intensify, phymatous changes, flushing, papules and pustules, telangiectasia, and/or ocular manifestations comprising lid margin telangiectasia, interpalpebral conjunctival injection, spade-shaped infiltrates in the cornea and scleritis and sclerokeratitis. Other diagnostic features may include burning or stinging sensations on skin (e.g., centrofacial skin), edema, dryness and ocular manifestations. See, Thiboutot et al., J. Am Acad. Dermatol. (2020) 82: 1501-1510.

Rosacea occurs most commonly in adult life, between the ages of 30 and 60 years. It is very common in skin types 1-11 (according Fitzpatrick) and more common in Caucasians, with a prevalence of up to 5% in the U.S. and in Europe. It is estimated that from 10 to 20 million Americans have the condition.

In some embodiments, a method is provided for treating rosacea in a patient in need thereof. In some embodiments, the method comprises: administering to the patient a composition and/or foamable composition or foam as described herein, wherein the foamable composition or foam comprises a tetracycline antibiotic. In some embodiments, compositions and/or foamable compositions or foams comprising about 1 to about 5%, e.g., about 3% minocycline are administered topically once daily to the full face and other rosacea-affected areas of the body for 12 weeks. In some embodiments, compositions and/or foamable compositions or foams comprising 3% minocycline are administered topically once daily for 12 weeks. In some embodiments, the compositions and/or foamable compositions or foams are applied daily at a fixed time, e.g., about 1 hour before bedtime or about 2 hours before bedtime.

In some embodiments, the daily dose is no more than 150 mg of minocycline, e.g., about 140 mg, about 130 mg, about 120 mg, or about 110 mg. In some embodiments, the daily dose is about 106 mg of minocycline. In some embodiments, the cumulative maximal dose is no more than 12 g of minocycline, e.g., 11 g, 10 g, 9 g, 8 g, or 7 g. In some embodiments, the cumulative maximal dose is no more than 8.9 g of minocycline.

In some embodiments, provided is a method for treating rosacea in a patient in need thereof, the method comprising: administering to the patient a foamable composition or foam as described herein, wherein the foamable composition or foam is comprises a tetracycline-based antibiotic. In some embodiments, foamable compositions or foams comprising 3% minocycline and 0.3% adapalene are administered topically once daily to the full face and other affected areas of the body for 12 weeks. In some embodiments, foamable compositions or foams comprising 3% minocycline are administered topically once daily to the full face and other affected areas of the body for 12 weeks. In some embodiments, foamable compositions or foams comprising 0.3% adapalene are administered topically once daily to the full face and other affected areas of the body for 12 weeks. In some embodiments, the foamable compositions or foams are applied daily at a fixed time, e.g., about 1 hour before bedtime or about 2 hours before bedtime, to the full face and other affected areas of the body.

In one or more embodiments there is provided a method of treating a subject for rosacea comprising diagnosing a subject having rosacea as at risk for tissue damage, wherein the tissue damage comprises circumscribed permanent swelling/thickening of skin areas, typically the nose, and administering a composition disclosed herein.

In one or more embodiments there is provided a method of treating a subject for rosacea comprising diagnosing a subject having rosacea as at risk of developing ocular damage, wherein ocular damage comprises dryness, burning and stinging, light sensitivity, blurred vision, foreign body sensation, lid margin and conjunctival telangiectases, plugging of the meibomian glands, chalazia, chalazion affecting the eyelid, corneal inflammation and scarring and/or corneal perforation or loss of visual acuity, and administering a composition disclosed herein.

In one or more embodiments there is provided a method of treating a subject for rosacea comprising diagnosing a subject having rosacea as at increased risk of a growing number of systemic disorders comprising cardiovascular, gastrointestinal, neurological, autoimmune disease and/or cancer, and administering a composition disclosed herein.

In one or more embodiments there is provided a method of treating a subject for acne or rosacea, comprising diagnosing a subject having acne rosacea as at risk of mental or social damage, wherein said damage comprising social, psychological and/or emotional symptoms, and administering a composition disclosed herein. In one or more embodiments the psychological and emotional symptoms comprise depression and/or anxiety. In one or more embodiments the social symptoms interfere with social or occupational interactions.

### Other Topical Disorders

In one or more embodiments there is provided a method of treating a topical skin disorder.

In one or more embodiments are methods for treating a topical skin disorder in a subject, the method comprising topically administering to a subject in need thereof a therapeutically effective amount of a formulation provided herein.

In some embodiments the disorder includes at least one etiological factor selected from the group consisting of an infection, an inflammation, oxidative stress, neurodegeneration, and apoptosis.

In some embodiments, the disorder is one or more of dermatological pain, dermatological inflammation, dermatitis, bacterial skin infections, fungal skin infections, viral skin infections, impetigo, pruritis, cellulitis, folliculitis, rashes, trauma or injury to the skin, post-operative or post-surgical skin conditions, eczemas, actinic keratosis, psoriasis, dermatitis, contact dermatitis, atopic dermatitis, or skin scarring.

In some embodiments, a method for treating the topical disorder in a patient in need thereof is provided, the method comprising: administering to the patient a composition and/or foamable composition or foam as described herein, wherein the foamable composition or foam comprises a tetracycline antibiotic. In some embodiments, compositions and/or foamable compositions or foams comprising minocycline (e.g., about 3% minocycline) and adapalene (e.g., about 0.3% adapalene) are administered topically once daily (or some other appropriate dosing schedule) to the disorder-affected area(s) of the body as appropriate for about 1 to 12 weeks.

### EXAMPLES

The invention will be described in greater detail by way of specific examples. The following examples are offered for illustrative purposes and are not intended to limit the invention in any manner. Those skilled in the art will readily recognize a variety of non-critical parameters which can be changed or modified to yield essentially the same results. In one or more embodiments, the amounts in the examples should be read with the prefix “about”. When present and unless indicated otherwise, a second figure in the table represents a standard deviation.

### Materials

Exemplary possible ingredients suitable for the production of compositions and foamable compositions are disclosed herein, and in Table 1. Equivalent, and in some cases, similar materials from other manufacturers can also be used.

TABLE 1
Exemplary Ingredients Suitable for the Production of Foamable Compositions

| | | | Melting | |
|---|---|---|---|---|
| | Commercial | | Temperature | |
| Chemical Name | Name | Supplier | ° C | Function |
| Coconut oil | Coconut oil | Henry-Lamotte | 23-26 | Emollient |
| Light mineral oil | Pionier 2076p | Hansen & | <0 | Emollient |
| | | Rosenthal | | |
| Isopropyl myristate | IPM | Merck | 3 | Emollient |
| Soybean oil | Soybean oil | Henry-Lamotte | −10 to −16 | Emollient |
| Stearic acid | Kolliwax S Fine | BASF | 70 | Foam adjuvant |
| Docosanol | Lanette 22 | BASF | 64-67 | Foam adjuvant |
| Stearyl alcohol | Kolliwax SA | BASF | 59 | Foam adjuvant |
| Cetostearyl alcohol | Kolliwax CSA 50 | BASF | 48-56 | Foam adjuvant |
| Myristyl alcohol | Kolliwax MA | BASF | 38 | Foam adjuvant |
| Hydrogenated | Kolliwax HCO | BASF | 83-88 | Wax |
| castor oil | | | | |
| (“HCO”) | | | | |
| White wax | White wax, bees | Strahl & Pitsch | 61-65 | Wax |
| (beeswax) | | | | |
| Emulsifying Wax, | Emulsifying Wax, | Croda/ | 50-54 | Wax 1 |
| NF | NF | Spectrum | | |
| Paraffin 57-60 | Paraffin wax | Merck | 57-60 | Wax |
| Cyclomethicone | ST- | Dow Corning | <−40 | Emollient |
| | cyclomethicone | | | |
| | 5NF | | | |
| Glycerol behenate | Glycerol | BSC | 83 | glyceride |
| | behenate | Distributers/ | | |
| | | GATEFOSSE | | |
| Carnauba wax | Carnauba wax | Kahlwax | 81-86 | Wax |
| Candelilla wax | Kahlwax 2039 | Kahlwax | 68.5-72.5 | Wax |
| (68.5-72.5) | | | | |
| Safflower oil | Linoleic acid | Henry-Lamotte | No | Emollient |
| | (C18:2) | | determined | |
| Corn oil | Linoleic acid | Henry-Lamotte | −18 | Emollient |
| | (C18:2), oleic acid | | | |
| | (C18:1) and | | | |
| | palmitic acid | | | |
| | (C16:0) | | | |
| Minocycline HCl | Minocycline HCl | Amri (Euticals | 205-210 | Active agent |
| (“MCH”) | micronized | SpA) | | |
| Minocycline HCl | Minocycline HCl | Hovione | 205-210 | Active agent |
| (“MCH”) | micronized | | | |
| Adapalene (“ADP”) | Adapalene | Excella | 328 | Active agent |
| Calcitriol | Calcitriol | Solvay | 111-115 | Active agent |
| | | Pharmaceutical | | |
| | | BV | | |
| Mometasone | Mometasone | | 218-220 | Active agent |
| Hydrogenated | Hydrogenated | Parchem | 72-75 | Wax |
| cottonseed oil | cottonseed oil | | | |
| Hydrogenated | Hydrogenated | Anmol | 66-72 | Wax |
| soybean oil | soybean oil | | | |
| Octyldodecanol | Octyldodecanol | | 1 | Emollient |
| Diisopropyl adipate | Diisopropyl | | <0 | Emollient |
| | adipate | | | |
| Propellant AP-70 | Propellant AP-70 | Aeropres | | Propellant |

### Experimental Method: Foam Quality

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam; upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

### Experimental Method: Collapse Time

Collapse Time, which is a measure of thermal stability, is measured by dispensing a given quantity of foam and photographing sequentially its appearance over time while incubating at 36° C.. The collapse time is defined as the time when the foam height reaches 50% of its initial height. However, if the foam takes longer than a threshold time, e.g., 180 seconds (s), to collapse to 50% of its initial height, then the collapse time may be recorded as >180 s. By way of illustration one foam may remain at 100% of its initial height for three minutes, a second foam may collapse to 90% of its initial height after three minutes, a third foam may collapse to 70% of its initial height after three minutes, and a fourth foam may collapse to 51% of its initial height after three minutes. Nevertheless, in each of these four cases the collapse time is recorded as >180 seconds. For practical purposes a foam is more easily applied to a target area if the majority of the foam remains intact for a reasonable period of time at 36° C. e.g., for more than 100 seconds, or more than 180 seconds. If, for example, the foam is reduced to 50% of its original height after 100 s, it would be recorded as having a collapse time of 100 s.

### Experimental Method: Drainage

The rate of drainage can be measured in a similar way to the collapse time. In the collapse time method, the foam is observed and filmed for a set period of time, e.g., 180 seconds. The height of the foam is measured against a marked ruler and any changes are recorded. The foam quality, as described above, is also observed throughout and any change in quality is noted. If a reduction in quality is observed, e.g., from Good to Fairly Good or from Excellent to Good, then significant drainage is considered to have occurred and the approximate time point when this change has been noted is said to be the drainage time.

### Experimental Method: Time to FG

A picture of the foam is taken at time intervals: 10, 30, 60, 90, 120, 150 and 180 seconds. At each time point the foam quality is assessed visually. The time taken by the foam to get to FG quality is recorded.

### Experimental Method: Viscosity/Rheology

Rheology analysis on placebo samples is made using a DHR3 rheometer (which provides similar measurements to the DHR2 rheometer used with API samples) from TA instruments. Rheology analysis on active samples is made using a DHR2 rheometer from TA instruments. The geometry used is a 40 mm parallel steel plate using a 1000 μm gap with temperature controlled by a Peltier bottom plate. Rotational measurements are made to obtain the viscosity at 36 s⁻¹. Oscillatory measurements are made to obtain the viscoelastic parameters. All measurements are made within the linear viscoelastic region. The elastic modulus G′ is obtained from the frequency sweep where the values are independent from the frequency. The flow point is obtained after a temperature sweep from 25° C. to 90° C. is performed. The flow point is the temperature at which G″ becomes higher than G′ and corresponds to the temperature at which the system starts flowing. Unless otherwise stated viscosity/rheology of the pre-foam formulation (PFF) is provided. It is not practical to try and measure the viscosity of the foamable formulation with propellants since they have to be stored in sealed pressurized canisters or bottles.

### Experimental Method: Hardness

An LFRA100 instrument (Brookfield Engineering Laboratories, Inc, USA) is used to characterize hardness. A probe (Aluminum cylinder probe; (dimensions: 25.5 mm, diameter; 6.4 mm height; Agentek)) is inserted into the test material. The resistance of the material to compression is measured by a calibrated load cell and reported in units of grams on the texture analyzer instrument display. Preferably at least three repeat tests are conducted. The textural characteristics of a dispensed foam can affect the degree of dermal penetration, efficacy, spreadability and acceptability to the user. A lower resistance to compression indicates a softer foam. Note: the foam sample is dispensed into an aluminum sample holder (Aluminum sample holder (dimensions: inner diameter: 38 mm, depth: 28.7 mm; Agentek)) and filled to the top of the holder.

### Experimental Method: Adhesion

Adhesion or adhesiveness is measured. Adhesiveness is defined as the force (g) needed to overcome attraction between two surfaces which are in contact. Measurements are made using the LFRA Brookfield Texture Analyzer. The two surfaces can be sections of artificial, actual tissue, or skin, and measure about 2×2 cm. During the measurement, one surface is positioned in the center of a Petri dish and the other surface is attached to the base of texture analyzer probe. A sample of pre-foam or foamable composition is spread uniformly on the surface that is on the Petri dish. The probe is moved down and up, first bringing the two sections into contact, then separating them. The Texture Analyzer measures the force for separating the surfaces, wherein the adhesive force is expressed as a negative force with the force to bring the two sections in contact as a positive force.

### Experimental Method: Differential Scanning Calorimetry (DSC)

Melting thermograms are obtained by differential scanning calorimetry using a DSC 250 or 2500 TA instrument where the melting temperatures (T_{M}) are obtained on the maximum peak of the different endotherms using the TRIOS software (TA instruments). Samples are weighed into hermetic aluminum pans (≈9-11 mg) and equilibrated at 25° C. for 5 min and then heated to 90° C. at 5° C./min.

### Experimental Method: Microscopy

Polarized optical microscopy: Photomicrographs of placebo samples are taken throughout a heating cycle from 25° C. to 80° C. The microscope used is an Olympus BX51, the camera is a Digital Hitachi Camera and the heating stage is a Linkam stage. Photomicrographs of active samples are taken using ZEISS Axioscope 7, equipped with Axiocam 305 digital camera and ZEN software. Heating was performed with Linkam hotstage with Linkpad touchscreen.

### Experimental Method: Small Angle X-ray Scattering (SAXS)

SAXSLAB GANESHA 300-XL (Skovlunde, Denmark), is used to measure the small angle x-ray scattering. Cu Kα radiation is generated by a Genix 3D Cu-source (operated at 47 mV and 0.55 mA) with integrated monochromator, 3 pinholes for collimation and a two-dimensional Pilatus 300K detector. The distance between the sample and detector is measured at two different configurations: 350 mm and 50 mm. The q range is between 0.012 to 0.67 Å⁻¹ and between 0.05 to 1.8 Å⁻¹ respectively.

### Experimental Method: Wide Angle X-ray Scattering (WAXS)

Wide-angle X-ray (WAXS) diffraction patterns of samples are obtained at room temperature with an X-ray diffractometer (Panalytical, X'Pert Pro; Almelo, The Netherlands) with CuKaR X-rays (λ=1.54 A) operating at 35 KV and 30 mA. Angular scans are obtained from 3° to 35° using a step size of 0.016° and a scan speed of 0.046°/s.

### Experimental Method: Isolating Crystals

Formulations prepared by a continuous heating-cooling process or a holding process are heated to a temperature of 50° C. At this temperature most crystals observed correspond to hydrogenated castor oil and display a spherulite structure (for a continuous heating-cooling process) or a nonuniform structure (for a holding process). Analysis of these different structures is performed by light microscopy (Olympus BX51, Camera: Digital Hitachi and heating stage: Linkam stage).

### Propellant Effect:

Unless otherwise stated viscosity/rheology, DSC, small angle X-ray scattering, wide angle X-ray scattering and microscopy are performed on pre-foam formulations (PFF), e.g., foamable compositions without the addition of propellant. In order to simulate the foamable formulations with propellant an equivalent weight of heptane (a low volatile hydrocarbon) is added to and mixed with the pre-foam formulation and left overnight. The tests are then performed as described above.

### Experimental Method: Shakability

For formulations tested (other than samples evaluated for a period of 6 months (Example 6) as described below), samples are loaded onto a shaker and shaken with increasing time (s) and frequencies (Hz) through six different levels until the samples reach optimal shakability (i.e., when one can hear and feel the material movement inside the canister as compared to the control sample). The testing level at which each sample reaches comparable shakability with the control sample is recorded. A result of testing levels 0-3 indicates “shakable (S)”, a result of testing levels 4-6 indicates “moderately shakable (M)”. If the sample completes testing level 6 and is still not shakable it should be assigned a result of testing level “6+” and indicates a “Non-shakable (N)”.

For samples evaluated for a period of 6 months (Example 6) shakability represents the degree to which the user is able to feel and/or hear the presence of the liquid contents when the filled pressurized canister is shaken. Shaking is done with normal mild force without vigorous shaking or excessive force. When the user cannot sense the motion of the contents during shaking the product may be considered to be non-shakable (N), when the user can moderately sense the motion of the contents during the shaking the product may be considered moderately shakable (M). When the contents are flowable during shaking the product may be considered Shakable (S). In one or more embodiments shaking is required for affecting dispersion of the contents. In some embodiments shaking is not required for affecting dispersion of the contents.

### Experimental Method: Sebum Physical Properties

The impact of tested formulations (Table 12b) on the physical properties of human sebum is analyzed.

Differential Scanning Calorimetry and light microscopy are utilized in this study. Artificial sebum formulation manufactured by Pickering Laboratories, Mountain View, CA, (product code 1700-0700, lot #0805019) is used for this study as a model because it closely matches the characteristics of human physiological sebum. The composition of Pickering artificial sebum is presented in Table 12b.

The mixtures of sebum with the test formulations are prepared as follows: the formulation of interest is weighed out and placed together with sebum into a glass test tube at ambient temperature, mixed manually with a stainless-steel spatula and then vortexed for 1 minute. For foam tested formulations, the foam is dispensed from the pressurized canister into a beaker and is manually collapsed by mixing with a glass rod. Then, the collapsed foam is mixed with sebum in the same way as described above. The resulting mixtures are tested by differential scanning calorimetry (DSC). Signal min x measurements are taken for each endotherm peak and represent the melting temperature of the evaluated sample. The DSC thermogram of the model sebum used in this study exhibits main broad melting endotherm with the signal min x at about 37° C. and several other minor endotherms at lower temperatures. It is the endotherm located in the temperature range close to the skin temperature, which is the focus for evaluation of follicular delivery.

The differential scanning calorimeter DSC250 or DSC2500, TA Instruments, New Castle, DE is used for DSC experiments. The sample is placed into a T-zero aluminum pan and crimped with a T-zero hermetic lid. The samples are cooled to 0° C. at 5° C./min, held at 0° C. for 5 min and then heated to 60° C. with a heating rate of 5° C./min.

The light microscopy experiments are performed on a Nikon Eclipse 50 microscope equipped with an s-polarizer and hot-stage accessory. A small amount of sebum and the formulation of interest are placed on a microscope slide side-by-side, in contact with each other but not mixed. A cover glass is placed on top. The slide is mounted on a hot stage and the temperature is raised from 25° C. to 35° C. at 2° C./min and then held at 35° C. A video is recorded, and photomicrographs are taken.

### In-Vitro Release Testing (IVRT)

Release test is performed using a Franz-cell apparatus. The tested formulation is placed on a suitable membrane, and a suitable receptor fluid is placed in the receptor chamber. The concentration of the active agent in the receptor fluid is measure over time, and the release rate is calculated.

### Experimental Method: Skin Penetration (Flow-Through Diffusion Cell System)

Skin penetration studies are performed e.g. with freshly excised abdominal human skin, utilizing a MedFlux-HTTM flow-through diffusion cell system. The exposed dosing area of about 1 cm² is dosed with approximately 10 mg of the formulation tested. The receptor solution (35 mM EDTA in 130 mM Sodium Acetate pH 5.0+0.01% Brij O20) continuous flow is set at 10 μL/min directly under the skin. The receptor solution is collected into 96-well plates at 2-hour intervals for 24 hours and then analyzed by Liquid chromatography-mass spectrometry (including two mass spectrometry detectors; LC-MS/MS) to determine the concentration of active agent (e.g. minocycline and/or adapalene) in the receptor solution. Following 24-hour exposure, the surface of the skin samples is cleaned and then tape-stripped 5 times to remove the excess formulation and the upper layers of stratum corneum. The skin is then separated into dermis and epidermis by heat treatment. The skin samples are extracted and the amount of active agent present in the skin layers is determined by LC-MS/MS. In some experiments, the skin samples are treated with SurgiSeal to extract sebaceous appendages, and the sebaceous appendages, dermis and remaining epidermis are analyzed for the active agent.

### Experimental Method: Skin Penetration (Vertical Franz Diffusion Cells)

Transdermal penetration of active agent(s) is tested using the Franz cell in-vitro diffusion system. This system is commonly used to test the delivery of drugs through the skin from semisolid topical dosage forms. Pig skin is used according to the OECD Draft New Guideline 428, since pig skin shows similar permeation characteristics to human skin.

About 5-6 Vertical Franz diffusion cells are used (PermeGear, 1.77 cm² area, 14 ml receptor fluid) to test the formulation while one cell is used as a “negative control” (without any applied sample). Approximately 500 mg of product is placed in each cell. (Note: The amount or formulation per surface area of skin used here is about three times more than the amount that might be applied clinically).

The receiving compartments are sampled at baseline and at 3-, 6-, 9-, and 24-hours following application. At the 24-hour time point the skin is processed as follows:

Residues of materials are removed from the skin using filter paper. The skin is then stripped successively using 20 pieces of adhesive tape “Scotch Magic® Tape”, 3M.

The first piece of adhesive tape is discarded. The second through tenth pieces of adhesive tape are transferred into a vial with 3 mL of extraction solution and labeled “Stratum Corneum 1.” The eleventh through twentieth pieces of adhesive tape are transferred into a different vial with 3 mL extraction solution and labeled “Stratum Corneum 2.”

The circular skin area (1.77 cm²) is cut and transferred to a 3 mL extraction solution (Viable skin—VS samples) vial.

The amount of active agent present is determined chromatographically.

### Experimental Method: Susceptibility Studies

Assessment for the formulation tested and comparator antibiotics is performed and interpreted according to Clinical & Laboratory Standards Institute (CLSI) guidance for agar and broth methodologies. C. acnes isolates are tested at International Health Management Associates, Inc. (IHMA). A subset is characterized by whole genome sequencing to determine multi-locus sequence type (MLST) and resistance mutations. Initial minimal inhibitory concentration (MIC) range for quality control strain Bacteroides fragilis ATCC 25285 is established. Minimal bactericidal concentration (MBC) is determined for clinical strains of C. acnes and B. fragilis.

### Experimental Method: Resistance Development Studies

Spontaneous resistance frequency to the formulation tested is evaluated by direct plating on antibiotic containing medium after single exposure. The development of further resistance in C. acnes strains with reduced susceptibility to minocycline (second-step mutants) is also assessed. Resistance development of C. acnes to minocycline is also assessed upon serial passage at sub-minimal inhibitory concentration (MIC) levels over a 30-day period.

### Experimental Method: Infrared Spectroscopy

Infrared spectroscopy model Bruker Vertex 70 spectrometer (Bruker Optics, Billerica, MA, USA) coupled to a microscope Hyperion 20001R (Bruker Optics, Billerica, MA, USA) with heating/cooling stage (Linkam, Surrey, UK Linkam T95) connected to a temperature control system (LTS 350; Linkam Scientific Instruments, Ltd.) and a tank of liquid nitrogen is used to measure the infrared spectra. Spectra are detected in transmission mode using a 15× objective and 32 scans in the interval of λ from 400 cm⁻¹ to 4000 cm⁻¹ with a resolution of 4 cm⁻¹. The temperature control system is used to maintain the heating and cooling rates of samples. The measurements are obtained using the Opus 7.2 software (Bruker Optics, Billerica, MA, USA).

A drop of sample is carefully placed on to a barium chloride slide and a cover slide is gently placed on top. Afterwards a heating ramp is set at 5° C./min from 25° C.-90° C. The infrared measurements are performed during the heating step (at 25° C., 35° C., 50° C., 60° C., 70° C., 80° C. and 90° C.).

To determine the frequency of different FTIR bands, an infrared spectroscopy model Bruker Vertex 70 spectrometer (Bruker Optics, Billerica, MA, USA) is coupled to a microscope Hyperion 20001R (Bruker Optics, Billerica, MA, USA) with heating/cooling stages (Linkam, Surrey, UK Linkam T95) connected to a temperature control system (LTS 350; Linkam Scientific Instruments, Ltd.) and a tank of liquid nitrogen. Spectra are detected in transmission mode using a 15×objective and performing 32 scans in the interval of λ from 400 cm⁻¹ to 4000 cm⁻¹ 1 with a resolution of 4 cm⁻¹. Measurements are obtained using the Opus 7.2 software (Bruker Optics, Billerica, MA, USA). An ATR (Attenuated Total Reflectance) measurement was used for the HCO neat (powder) instead of the microscope, since the ATR is more suitable for powders. A drop of sample is placed onto a Barium fluoride slide then a cover slide from the same material is placed carefully on top.

### Experimental Method: Concentrating Tmh Crystals

Samples containing Tmh crystals are prepared and placed into a 2-ml Eppendorf tubes equipped with 0.45 μm PTFE filters and centrifuged at 12000 rpm for 10 minutes at 55° C. The upper layer (oil fraction) is removed and the precipitant which contains the concentrated Tmh crystals is collected and analyzed.

### Experimental Method: Breaking of Tmh Crystals

Samples containing Tmh crystals are sonicated at 50° C. for 15 minutes, then a small aliquot was placed between glass slides and pressed with spatula to attempt to break up any aggregates. The samples are observed by polarized light microscopy at 50° C. with a 20× and a 50× objective.

### Experimental Method: Measurement of Percentage Area

Aliquots of formulation samples containing crystals are randomly picked out. Aliquots (e.g., two) are placed on a slide and imaged by light microscopy. The image is captured by a microscope with an image of 645 μm×452 μm. A software program calculates the area of the total picture area occupied by each crystal. The percentage area occupied by the crystals is determined with the average of 3 different areas of the image.

### Experimental Method: Local and Systemic Toxicity and Toxicokinetics in Gottingen Minipigs

Objective: The objective of this study is to characterize local and systemic toxicity, and toxicokinetics of minocycline and adapalene foam, along with adapalene as a comparator, when administered via dermal application to Gottingen Minipigs once daily for up to 13 weeks (91 days), and to assess the delayed onset or recovery of any findings following a 28-day non-dosing observation period. The study design is provided in table 2 below:

TABLE 2
Experimental Design

| | | | | Minocycline | Adapalene | | | |
|---|---|---|---|---|---|---|---|---|
| | | Minocycline | Adapalene | Dose | Dose | Dose | | |
| Group | | Dose Level | Dose Level | Concentration | Concentration | Volume | No. of Animals | |
| No. | Treatment | (mg/kg/day) | (mg/kg/day) | (mg/g) | (mg/g) | (g/kg/day) | Males | Females |
| 1ª | Untreated | NA | NA | NA | NA | NA | 4 | 4 |
| | (naïve) | | | | | | | |
| 2^{b} | Placebo | 0 | 0 | 0 | 0 | 0.25 | 6^{c} | 6^{c} |
| | foam | | | | | | | |
| 3 | Adapalene | 0 | 0.75 | 0 | 3 | 0.25 | 6^{c} | 6^{c} |
| | 0.3% foam | | | | | | | |
| 4 | Minocycline | 7.5 | 0.25 | 30 | 1 | 0.25 | 4 | 4 |
| | Hydrochloride | | | | | | | |
| | 3%, | | | | | | | |
| | Adapalene | | | | | | | |
| | 0.1% foam | | | | | | | |
| 5 | Minocycline | 7.5 | 0.75 | 30 | 3 | 0.25 | 6^{c} | 6^{c} |
| | Hydrochloride | | | | | | | |
| | 3%, | | | | | | | |
| | Adapalene | | | | | | | |
| | 0.3% foam | | | | | | | |
| 6 | Minocycline | 7.5 | 1.50 | 30 | 6 | 0.25 | 6^{c} | 6^{c} |
| | Hydrochloride | | | | | | | |
| | 3%, | | | | | | | |
| | Adapalene | | | | | | | |
| | 0.6% foam | | | | | | | |

^{a}Animals in Group 1 are untreated control animals and are treated in the same manner as the treated animals except no test article, vehicle, or control article was administered.
^{b}Animals in Group 2 are administered the vehicle, FCD105 vehicle foam (0% minocycline and 0% adapalene).
^{c}Two animals/sex are maintained on study for a 28-day recovery period.
NA—Not Applicable

The following parameters and endpoints are evaluated in this study: mortality, clinical observations, evaluation of skin reaction, and body weight, ophthalmoscopic, electrocardiographic examinations, clinical pathology parameters (hematology, coagulation, clinical chemistry, and urinalysis), toxicokinetic parameters, gross necropsy findings, organ weights, and histopathologic examinations.

Cage Side Observations

All animals are observed for morbidity, mortality, injury, and the availability of food and water twice daily, once in the morning and once in the afternoon. Animals are not removed from the cage during observation, unless necessary for identification or confirmation of possible findings.

Detailed Clinical Observations

The animals are removed from the cage, and a detailed clinical examination of each surviving animal is performed weekly during the study. On occasion, clinical observations are recorded at unscheduled intervals. The unscheduled examinations performed during the acclimation period are not reported but are maintained in the study file. The observations include, but are not limited to, evaluation of the skin, fur, eyes, ears, nose, oral cavity, thorax, abdomen, external genitalia, limbs and feet, respiratory and circulatory effects, autonomic effects such as salivation, and nervous system effects including tremors, convulsions, reactivity to handling, and unusual behavior.

Detailed Cageside Clinical Observations

A detailed cageside clinical examination is performed once daily at 4 hours (±30 minutes) post-dose during the study. Each surviving animal is examined visually while still in the cage for clinical signs of disease, toxicity, and injury.

Evaluation of Skin Reaction

The test site is scored for erythema/eschar and edema once daily prior to dosing during Week 1 and once weekly (at 1 hour post-dose on days of dosing) thereafter.

The scores presented in Text Tables 3 and 4 are based upon the Draize scale for scoring skin irritation.

TABLE 3
Erythema and Eschar Formation

| Score | Observation |
|---|---|
| 0 | No erythema |
| 1 | Very slight erythema (barely perceptible) |
| 2 | Well-defined erythema |
| 3 | Moderate to severe erythema |
| 4 | Severe erythema (beet redness) to slight eschar formation |
| | (injuries in depth) |

Maximum possible score = 4

TABLE 4
Edema Formation

| Score | Observation |
|---|---|
| 0 | No edema |
| 1 | Very slight edema (barely perceptible) |
| 2 | Slight edema (edges or area well-defined by definite raising) |
| 3 | Moderate edema (raised approximately 1 mm) |
| 4 | Severe edema (raised more than 1 mm and extending beyond |
| | area of exposure) |

Maximum possible score = 4

Body Weights

Body weights for all surviving animals are measured and recorded on the day of receipt, Day-5 (female animals only), prior to randomization (Day-1), and weekly during the study. The body weights recorded on the day of receipt and Day-5 are not reported but are maintained in the study file.

Body weight changes are calculated for animals between each weighing interval.

Food Consumption

A daily qualitative assessment of food intake/appetite is performed for all surviving animals as part of the twice daily cage side observations. Quantitative food consumption measurements are not conducted.

Ophthalmic Examinations

Ophthalmic examinations are conducted pretest and prior to the terminal necropsy by an ophthalmologist.

Electrocardiography Examinations

Electrocardiographic examinations are performed on all surviving animals pretest, pre-dose and 1 to 2 hours post-dose during the last week of dosing, and once at the end of the recovery period. Insofar as possible, care is taken to avoid causing undue excitement of the animals before the recording of electrocardiograms (ECGs) in order to minimize extreme fluctuations or artifacts in these measurements. Standard ECGs (6 Lead) are recorded at 50 mm/sec. Using an appropriate lead, the RR, PR, and QT intervals, and QRS duration are measured and heart rate is determined. Corrected QT (QTc) interval is calculated using a procedure based on the method described by Fridericia. All tracings are evaluated and reported by a consulting veterinary cardiologist.

Laboratory Evaluations

Clinical Pathology

Clinical pathology evaluations are conducted on all surviving animals pretest and prior to the scheduled terminal and recovery necropsies. Bone marrow smears are collected and preserved.

Bioanalysis and Toxicokinetic Evaluation

Bioanalytical Sample Collection

Blood samples (approximately 2 mL) are collected from all surviving animals via the abdominal vena cava through the thoracic inlet for determination of the plasma concentrations of adapalene and minocycline (see Tables 5 and 6). The animals were not fasted prior to blood collection, with the exception of the intervals that coincided with fasting for clinical pathology collections.

TABLE 5
Bioanalysis Sample Collection Schedule

Sample Collection Time Points
(Time Post-dose) on Days 1 and 91

| Group No. | 0^{a} hr | 1 hr | 2 hr | 4 hr | 8 hr | 24 hr |
|---|---|---|---|---|---|---|
| 1^{b} | X | X | X | X | X | X |
| 2^{b} | X | X | X | X | X | X |
| 3 | X | X | X | X | X | X |
| 4 | X | X | X | X | X | X |
| 5 | X | X | X | X | X | X |
| 6 | X | X | X | X | X | X |

X = Sample is collected
^{a}Sample is collected before dosing.
^{b}Samples are collected from animals in Groups 1 and 2 at all time points for consistency. However, only the 1 hour samples are analyzed

TABLE 6
Bioanalysis Sample Collection Schedule

| | Recovery Sample Collection Time Points | | | |
|---|---|---|---|---|
| | (Time Post-dose from Day 91 Dose) on Days 99, 105, | | | |
| | 112, and 119 | | | |
| Group No. | 192 hr | 336 hr | 504 hr | 672 hr |
| 2 | X | X | X | X |
| 3 | X | X | X | X |
| 5 | X | X | X | X |
| 6 | X | X | X | X |

X = Sample is collected

Blood samples are collected in tubes containing K₂EDTA and placed on wet ice and centrifuged under refrigerated conditions. The resulting plasma is divided into 3 aliquots (100 μL in Aliquot 1 [minocycline], 500 μL in Aliquot 2 [adapalene], and any remaining plasma in Aliquot 3) in pre-labeled cryovials. All aliquots are stored frozen at −60° C. to −90° C.

Bioanalytical Sample Analysis

Samples are shipped on dry ice to Nuvisan GmbH Bioanalysis, Neu-Ulm, Germany, for analysis. All analytical work is conducted by Nuvisan GmbH Bioanalysis using an analytical method developed by that laboratory.

Toxicokinetic (TK) Evaluation

The TK parameters are determined for adapalene and minocycline from individual concentration-time data by the Testing Facility.

Terminal Procedures

Post-mortem study evaluations are performed on animals found dead, euthanized in extremis, or euthanized at the scheduled terminal (Day 92) and recovery necropsies (Day 120).

Method of Euthanasia

Euthanasia will be by euthanasia solution administration, under sedation if necessary (e.g. acepromazine and/or Telazol®), followed by a Testing Facility SOP approved method to ensure death, e.g. exsanguination.

Macroscopic

Necropsy examinations are performed under procedures approved by a veterinary pathologist. The animals are examined carefully for external abnormalities including palpable masses. The skin is reflected from a ventral midline incision and subcutaneous masses are identified and correlated with antemortem findings. The abdominal, thoracic, and cranial cavities are examined for abnormalities. The organs are removed, examined, and, where required, placed in fixative. All designated tissues are fixed in neutral buffered formalin, except for the eyes (including the optic nerve) and testes, which are fixed using a modified Davidson's fixative prior to placement in formalin. Formalin is infused into the lung via the trachea. A full complement of tissues and organs is collected from all animals.

Organ Weights

Body weights and protocol-designated organ weights are recorded for all surviving animals at the scheduled necropsies and appropriate organ weight ratios are calculated (relative to body and brain weights). Paired organs are weighed together.

Microscopic Evaluation

Fixed hematoxylin and eosin-stained paraffin sections from protocol-designated sections of tissues are processed to slide and all required slides are shipped under ambient condition to Charles River Laboratories, Inc., Frederick, Maryland for microscopic evaluation.

### Statistics

Table 7 defines the set of comparisons used in the statistical analyses described in this section.

TABLE 7
Statistical Comparisons

| Control Groop | Treatment Group |
|---|---|
| 1 | 2, 3, 4, 5, 6 |
| 2 | 3, 4, 5, 6 |
| 3 | 4, 5, 6 |

The raw data are tabulated within each time interval, and the mean and standard deviation are calculated for each endpoint by sex and group. For each endpoint, treatment groups are compared to the control group using the analysis outlined in Table 8.

However, because of the limited number of animals, statistical evaluations of recovery animal endpoints are not conducted.

TABLE 8
Statistical Analysis

| Endpoints | Type of Analysis |
|---|---|
| Body Weights | Analysis of Variance |
| Body Weight Change | with Adjustment |
| Hematology | for Multiple |
| Coagulation | Comparisons |
| Clinical Chemistry | |
| Urinalysis (Urine Volume, Specific Gravity, | |
| and pH) | |
| Organ Weights (Absolute Weights and Relative | |
| to Body and Brain Weights) | |
| ECG Intervals | |
| Creatine kinase | |

### Experimental Method: Isolation of Tmh Crystals

Formulation are prepared as described above. The crystals may optionally be concentrated by centrifugation. The crystals are isolated by manual breaking of Tmh crystals. Samples containing Tmh crystals are sonicated at 50° C. for 15 minutes, then a small aliquot is placed between glass slides and pressed with spatula to attempt to break up any aggregates. The samples are observed by polarized light microscopy at 50° C. with a 20× and a 50× objective.

### Experimental Method: Imaging of Crystals in the Present of a Solvent

A sample is mixed with a solvent in which the crystals are not soluble in approximately a 1:1: ratio. For example, 0.5 g of sample formulation that is stored at 5° C. is mixed with 0.5 ml of solvent (acetone/hexane; to separate between crystals), gently stirred and a drop is placed onto a glass slide and a cover slide is gently placed on top. Microscopic observation and analysis is made at different temperatures and during slow heating, e.g., from 20 to 75° C. at 1° C./min.

### Experimental Method: Compatibility Test

Mixtures of solid Minocycline HCl and Adapalene are added to neat soybean oil, neat corn oil or neat safflower oil. The exact amount of Minocycline HCl and Adapalene are determined for each sample by weighing the active ingredients with analytical balance. The mixtures are placed into glass screw cap vials, tightly closed and exposed for 3 weeks to 50° C. temperatures, protected from light. Following 3 weeks of exposure, the mixtures are equilibrated with ambient conditions and Minocycline, Adapalene and their degradation products are determined by HPLC, analyzing the complete samples. The extent of degradation of Minocycline and Adapalene is determined for each sample by comparison of amounts of Minocycline HCl and Adapalene recovered in each sample with the weights of Minocycline HCl and Adapalene used in preparation of corresponding samples. The content of degradation products is determined by an area percent ratio for each degradation product corresponding to the main peak of the corresponding active ingredient. The samples are evaluated for color alterations, as well.

TABLE 9

| Ingredient | Ingredient content, % w/w | | |
|---|---|---|---|
| Adapalene | 0.3 | 0.3 | 0.3 |
| Minocycline HCl * | 3.58 | 3.58 | 3.58 |
| Soybean oil | qs to 100 | | |
| Corn oil | | qs to 100 | |
| Safflower oil | | | qs to 100 |
| Bulk Total | 100 | 100 | 100 |

* 3.58% of Minocycline HCl corresponds to 3.0% of Minocycline

### Example 1

### General Manufacturing Procedures for a Gel or a Foam

The following procedures are used to produce gel or foam samples, in which only the steps relevant to each formulation are performed depending on the type and nature of ingredients used. Alternative processes are also described in other Examples and in the specification.

### a) Manufacturing by a Continuous Heating-Cooling Process:

Step 1: Hydrophobic solvents and solid compounds such as fatty alcohols, fatty acids and waxes are heated with mixing, to a temperature sufficient for a homogenous mixture to be visually observed.

Step 2: The formulation is cooled down to 35-40° C., then temperature sensitive components such as cyclomethicone and temperature sensitive active agents such as tetracyclines are added while mixing, until formulation homogeneity is visually observed. If they are to be included, sensitive active agents such as retinoids are added at 24-28° C. while mixing until formulation homogeneity is visually observed.

Step 3: The formulation is cooled down to 22-26° C..

Step 4: The formulation is mixed (for about 3 up to 24 hours) at 20-24° C.

Step 5: For gel compositions, the formulation is packaged in suitable containers. For foamable compositions, the formulation is packaged in aerosol canisters which are filled, crimped with a valve, and pressurized and mixed with a propellant (e.g., a hydrocarbon gas or gas mixture) as described below. Each cannister is equipped with an actuator suitable for foam dispensing. The canisters or containers are labeled.

b) Manufacturing with a Single Step Holding Process:

Step 1: Hydrophobic solvents and solid compounds such as fatty alcohols, fatty acids and waxes are heated with mixing, to a temperature sufficient for a homogenous mixture to be visually observed.

Step 2: The formulation is cooled down to a holding temperature, for example, about 54° C. and is mixed at this temperature for a holding period, for example, of 4 hours, unless indicated otherwise for a specific example.

Step 3: The formulation is cooled down to 35-40° C., then sensitive components such as cyclomethicone and sensitive active agents such as tetracyclines are added while mixing until formulation homogeneity is visually observed.

Step 4: The formulation is cooled down to 24-28° C.. Sensitive active agents such as retinoids are added (as dispersion in a hydrophobic solvent) under mixing until formulation homogeneity is visually observed.

Step 5: The formulation is cooled down to 22-28° C..

Step 6: The formulation is mixed (for about 3 up to 24 hours) at 20-24° C.

Step 7: For gel compositions (and pre-foam), the formulation is packaged in suitable containers. For a non-foam gel, for example, it can be packaged in a coated metal tube, which is sealed and capped. For foamable compositions, the formulation is packaged in aerosol canisters which is filled, crimped with a valve and pressurized and mixed with a propellant (e.g., a hydrocarbon gas or gas mixture) as described below. Each cannister is equipped with an actuator suitable for foam dispensing. Lastly, the canisters or containers are labeled.

### Mixing

The formulation is mixed during one or more or all steps as is needed to ensure the formulation is homogenous. Mixing may be a mixing means such as a propeller or stirrer or a homogenizer.

### Canisters Filling and Crimping:

Each aerosol canister is filled with the pre-foam formulation (“PFF”, i.e., foamable carrier) and crimped with valve, optionally using a vacuum crimping machine. The process of applying a vacuum will cause part of the oxygen present to be eliminated.

### Pressurizing:

Pressurizing is carried out using a hydrocarbon gas or gas mixture and shaken immediately thereafter. Such a process prepares the foamable composition as provided herein.

### Nitrogen and Vacuum

Each manufacturing process is carried out in a closed system with purging with nitrogen and mixing under vacuum.

### High Shear or Energy Input

During and following the holding step mixing using high shear or other methods that can input heat or energy into the crystals (e.g., as they pass through a homogenizer mixer) may be avoided or ameliorated or used only for a relatively short period so that the Tmh crystals are not substantially reduced or eliminated in the composition. Without being bound by any theory, mixing devices that generate localized heat in the manufacturing system may result in the Tmh crystals melting and or being converted into other crystalline forms such as spherulites.

### Example 1A—General Manufacturing Process for an Emulsion

The following procedures are used to produce an emulsion formulation and an emulsion foamable formulation.

Step 1: hydroxypropyl methylcellulose, xanthan gum and citric acid are added to water at ambient temperature and mixed until fully dissolved. The solution is heated to 60° C. while being mixed.

Step 2: To generate the oil phase caprylic/capric triglyceride is heated to 60°-70° C., stearic acid, glyceryl monostearate and ceteareth 20 are added and mixed until excipients fully dissolved.

Step 3: The oil phase is slowly added to the water phase and aggressively mixed to achieve homogeneous emulsion.

Step 4: The mixture is cooled down, while being mixed to 35°-40° C., then glycerin is added. pH is adjusted to 5.0-5.5 by either sodium hydroxide or by hydrochloric acid, if needed. The final blend is cooled down to 25° C. while being mixed.

Step 5: For foamable compositions, the formulation is packaged in aerosol canisters which is filled, crimped with a valve and pressurized and mixed with a propellant (e.g., a hydrocarbon gas or gas mixture) as described above.

### Example 2

### Mixtures Prepared by Stepwise Addition of Formulation Components

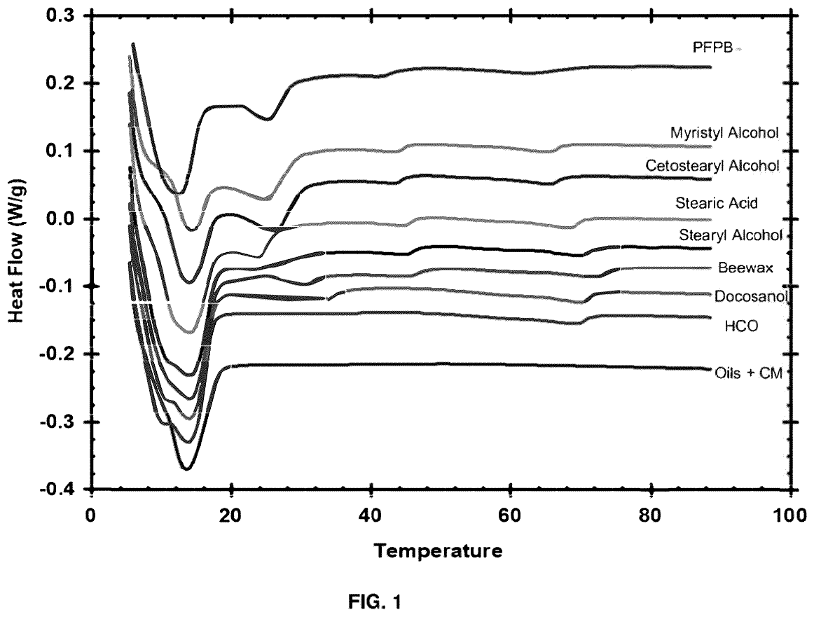

Different mixtures were created by stepwise addition of individual components in the order presented in Table 10A and in the amounts shown in Table 10B. Upon addition of each component, the mixture was first melted at 90° C. for 20 minutes, then crystallized by cooling (at 10° C./min) to 5° C. After 2 minutes at 5° C., the mixture was heated from 5° C. up to 90° C. (at 5° C./min). In this way, the melting profile of each of the components can be characterized (Table 10A and FIG. 1) and the complexity of the interactions of the components can be appreciated.

The results of such a stepwise process and its final composition can be seen in Tables 10A and 10B respectively.

For example, addition in step 2 of hydrogenated castor oil (HCO) to the oils in the formulations resulted in a major transition (TM4) appearing at about 69° C. However, DSC for the full formulation (step 9) showed HCO transition (TM4) at about 63° C., indicating a complex system of different crystal forms, with the presence of each crystal form affecting the crystallization profile of the other crystal forms. Without being bound by any theory, this difference in TM4 may also be related to the solubilization of HCO by other components of the formulation leading to the lower TM.

TABLE 10A

| STEP | SAMPLE | TM0 | TM1 | TM2 | TM2.1 | TM3 | TM4 |
|---|---|---|---|---|---|---|---|
| 1 | Oils + | | 14.17 | | | | |
| | cyclomethicone | | 0.33 | | | | |
| | (CM) | | | | | | |
| 2 | +Hydrogenated | 10.02 | 14.00 | | 33.40 | | 69.35 |
| | castor oil (HCO) | 0.20 | 0.24 | | 0.27 | | 0.45 |
| 3 | +Docosanol | 10.46 | 13.68 | 29.57 | 33.32 | | 69.58 |
| | | 0.13 | 0.27 | 0.17 | 0.07 | | 0.62 |
| 4 | +Beeswax | 10.75 | 14.03 | 22.39 | 31.31 | 45.97 | 72.06 |
| | | 0.13 | 0.24 | 0.19 | 0.27 | 0.16 | 0.07 |
| 5 | +Stearyl alcohol | 10.75 | 14.02 | 24.26 | 28.23 | 44.58 | 69.40 |
| | | 0.30 | 0.08 | 0.73 | 0.73 | 0.38 | 0.65 |
| 6 | +Stearic acid | | 13.78 | 23.79 | | 44.36 | 67.77 |
| | | | 0.11 | 0.08 | | 0.49 | 1.07 |
| 7 | +Cetostearyl | | 13.68 | 26.83 | | 43.41 | 65.72 |
| | alcohol | | 0.16 | 0.63 | | 0.07 | 0.06 |
| 8 | +Myristyl | | 14.45 | 24.68 | | 43.38 | 65.29 |
| | alcohol | | 0.04 | 0.40 | | 0.55 | 0.01 |
| 9 | Full placebo | | 12.36 | 24.78 | | 41.15 | 62.54 |
| | formulation | | 0.07 | 0.70 | | 0.54 | 0.77 |
| | (PFPB) | | | | | | |

TABLE 10B

| Component | % w/w |
|---|---|
| Soybean oil | 50 |
| coconut oil | 23.6 |
| Light mineral oil | 5.8 |
| Stearic acid | 3 |
| Docosanol | 1.1 |
| Hydrogenated castor oil | 2 |
| White wax (beeswax) | 2 |
| Stearyl alcohol | 1.5 |
| Cetostearyl alcohol | 3.5 |
| Myristyl alcohol | 2.5 |
| Cyclomethicone | 5 |

### Example 3

Formulations Manufactured with and without a Holding Process

Formulations containing either 2% or 1.2% hydrogenated castor oil (HCO) and active agents comprising a combination of minocycline HCl and adapalene (MCH+ADP) were prepared either by a continuous heating-cooling process or a holding process. Corresponding placebos without active agents were also prepared either by a continuous heating-cooling process or a holding process.

The microstructures and crystal fingerprint of the different formulations described in Tables 11 A-C were visualized by a microscope. As the presence of active agents masks the microstructures, images of placebo samples are presented instead, unless indicated otherwise.

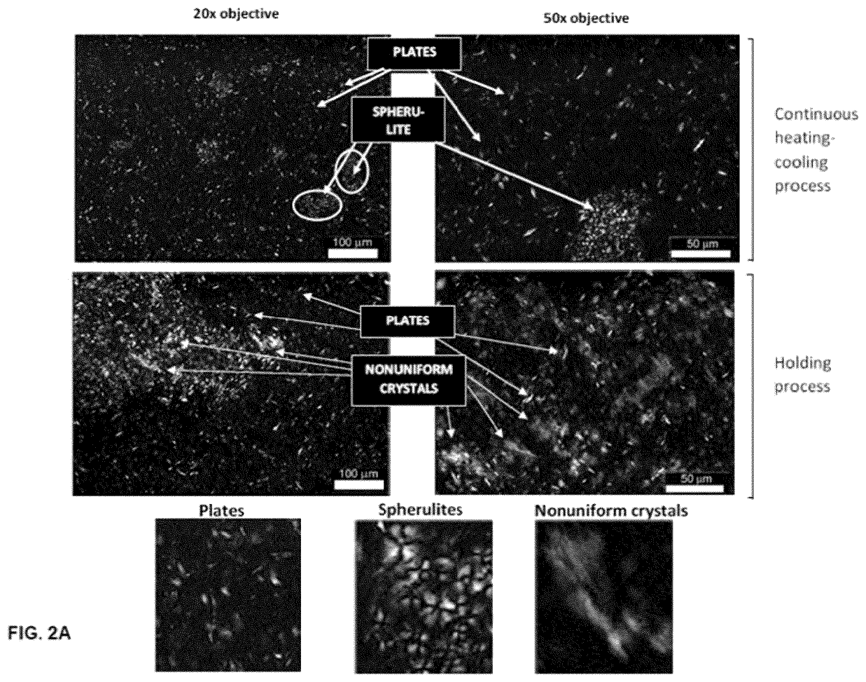

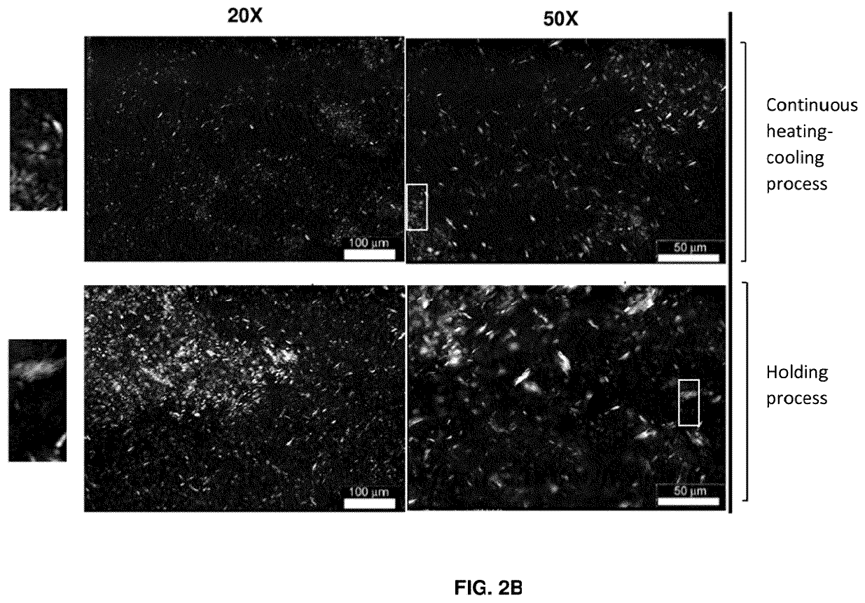

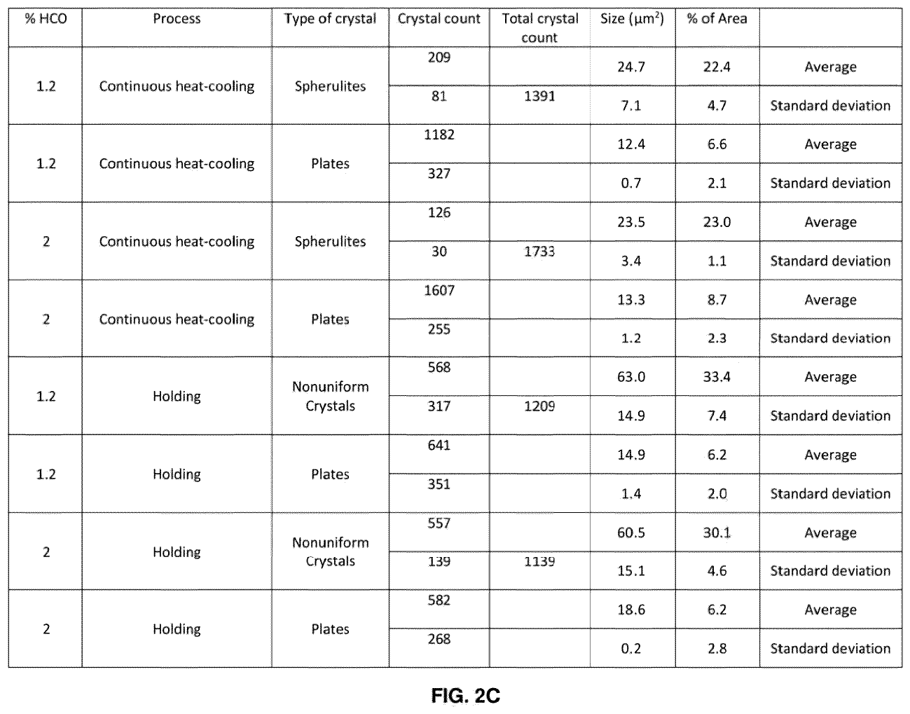

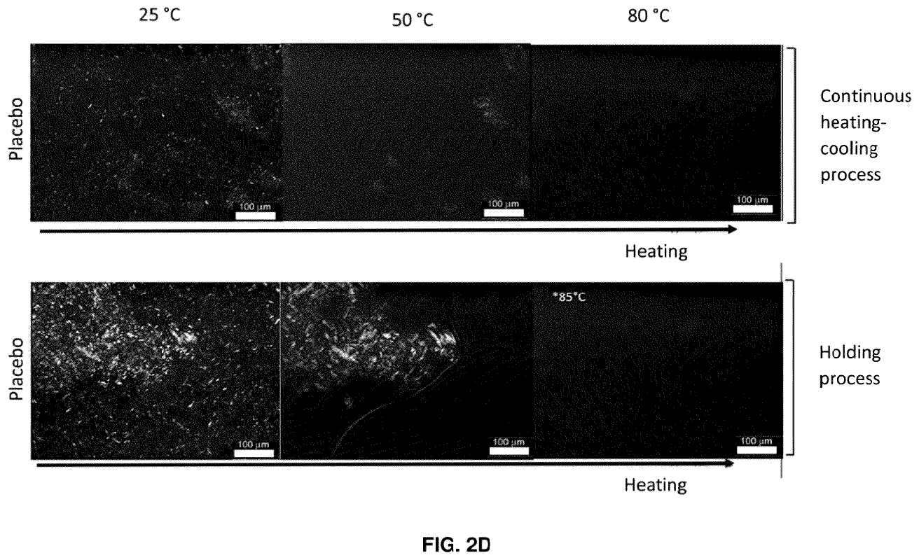

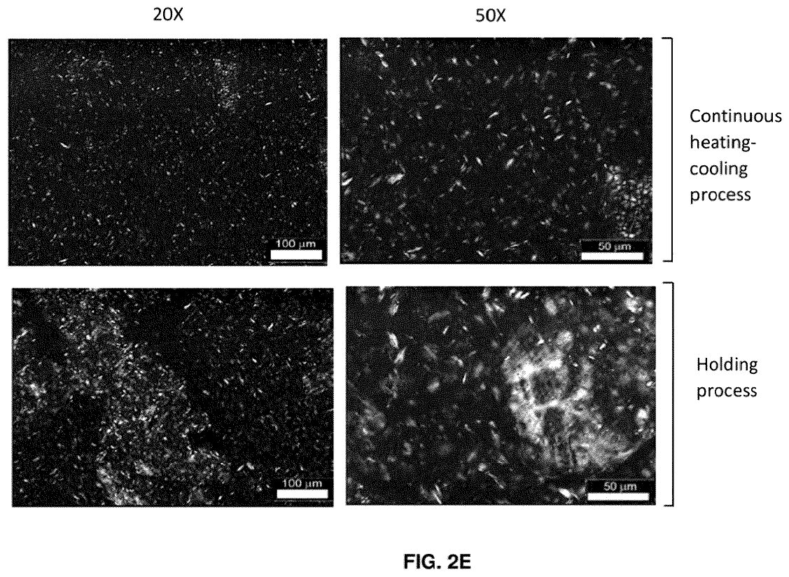

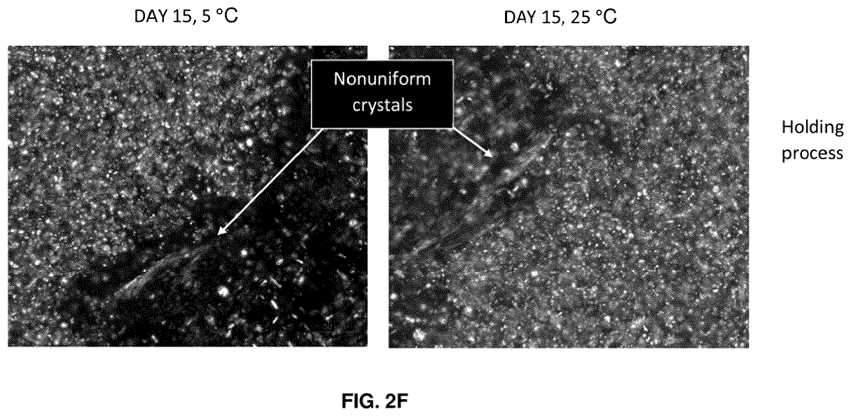

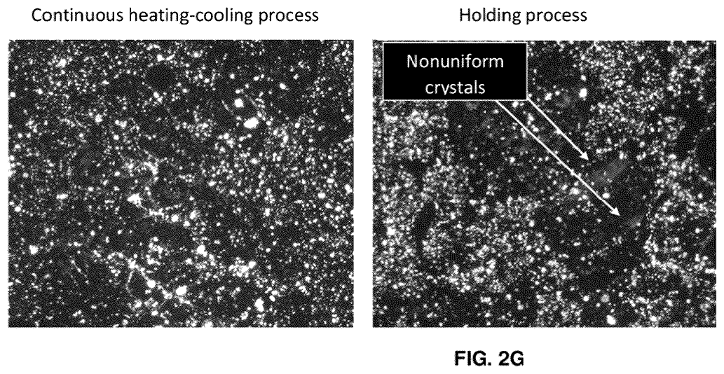

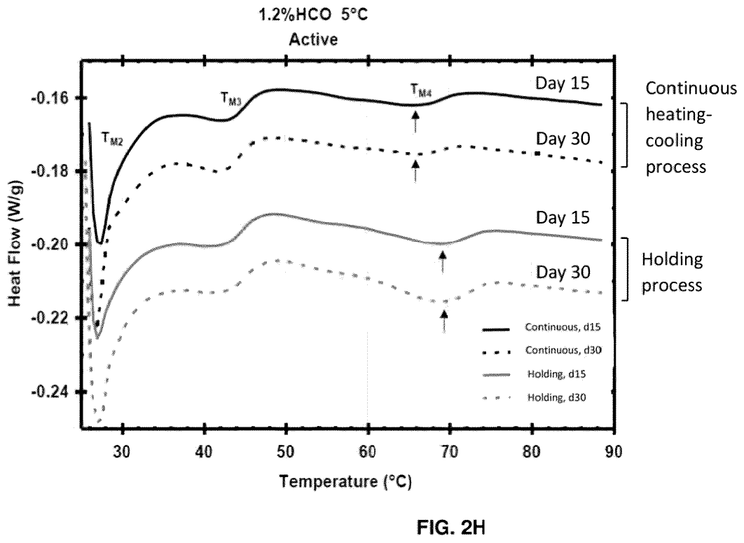

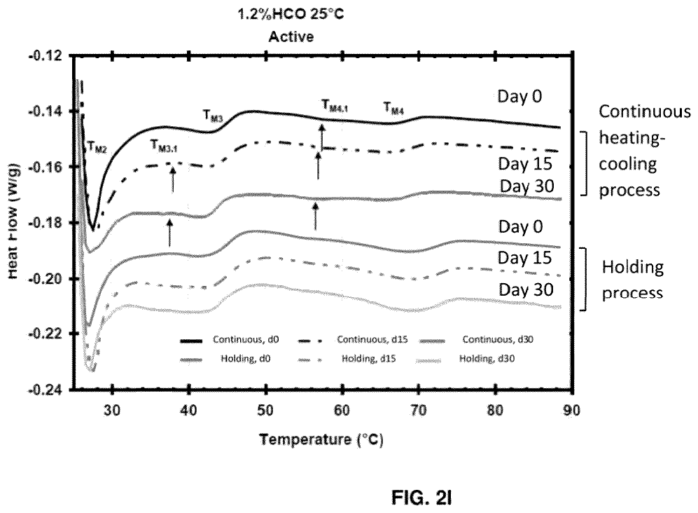

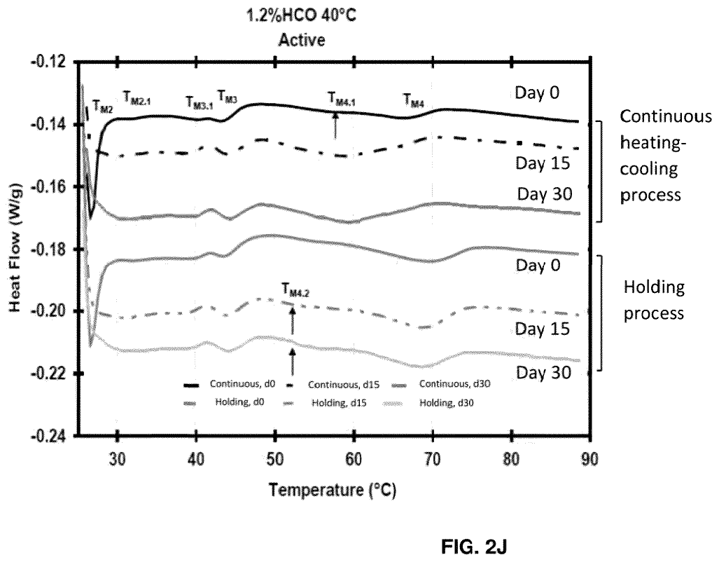

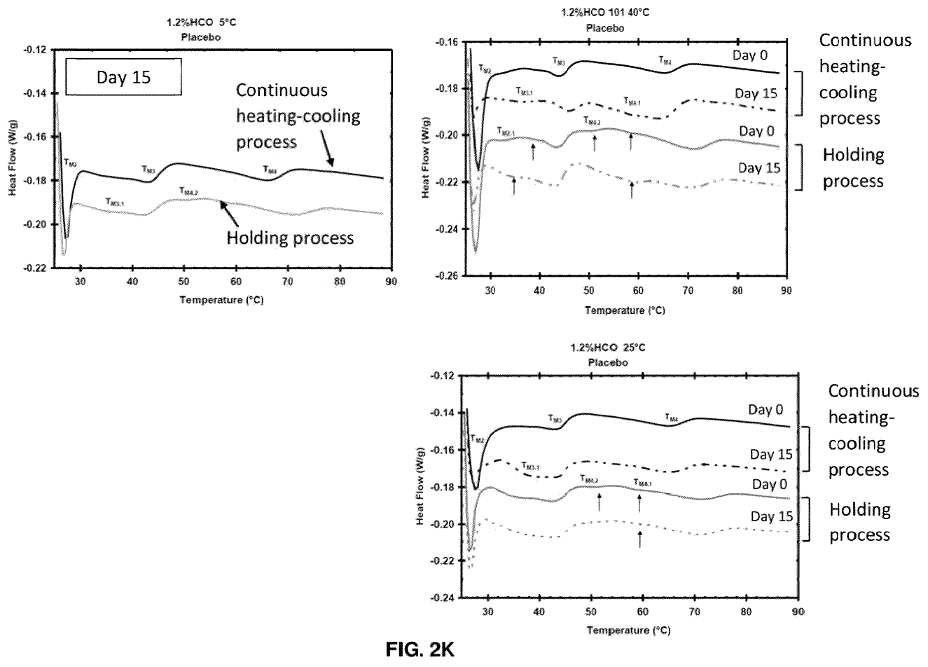

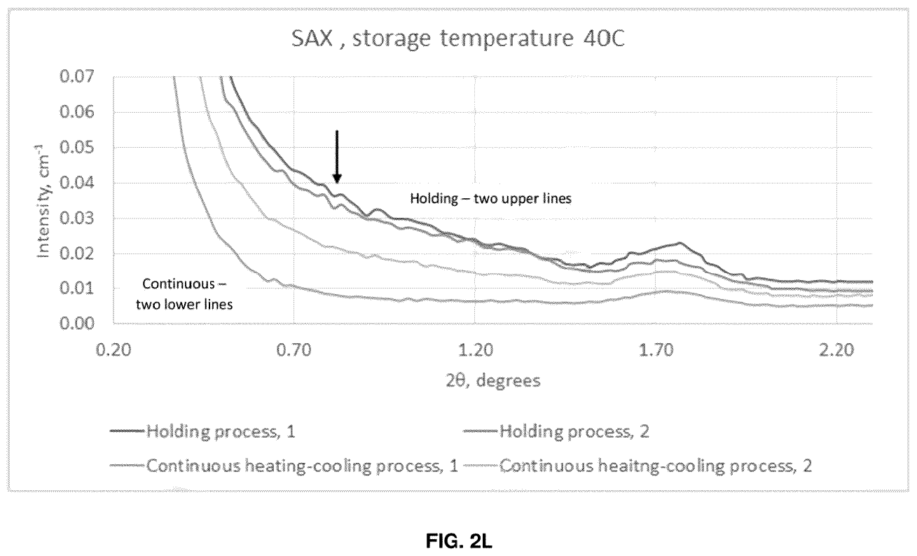

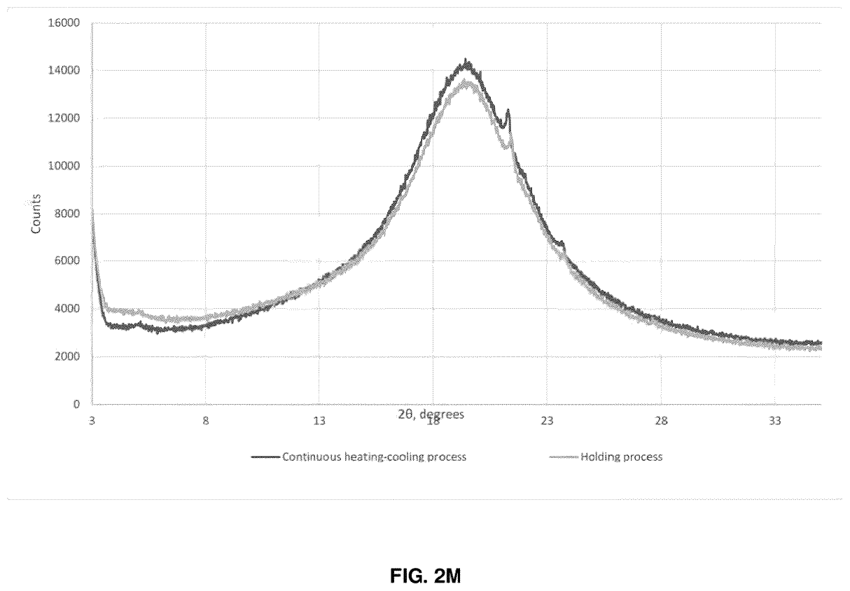

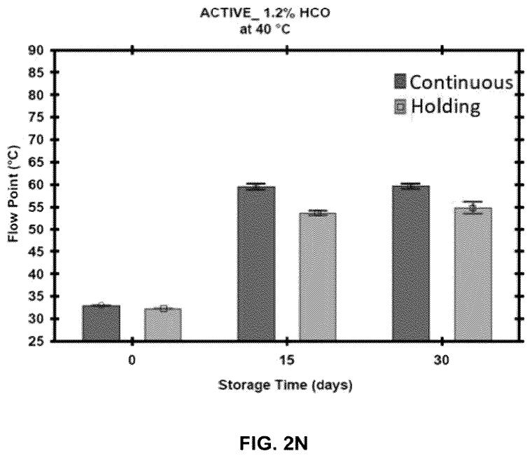

As can be seen in FIG. 2A, formulations prepared by a continuous heating-cooling process had plates and spherulites structures whereas those prepared by a holding process resulted in the appearance of plates and new nonuniform structures. Larger magnification of the different structures can be seen in FIG. 2A lower panel.

For example, on Day 0, placebo formulations with 1.2% HCO kept at 25° C. (FIG. 2B) showed clear differences in the microstructures and crystal fingerprint between formulations prepared by a continuous heating-cooling process and that prepared by a holding process. The continuous heating-cooling process resulted in plates and spherulites structures whereas the holding process resulted in plates of a bigger size, as well as nonuniform structures that were not observed in formulations prepared by a continuous heating-cooling process.

Photographs taken on Day 0 of placebo formulations with 1.2% HCO or 2% HCO kept at 25° C. and prepared by either a continuous heating-cooling process or a holding process were analyzed by image analysis software.

Analysis was performed on selected areas in which the majority of the crystals were either nonuniform, spherulites or plates. The area of each crystal was measured. The average area of each crystal type is presented. As shown in FIG. 2C, the Tmh crystals observed in formulations prepared by a holding process were the largest crystals (about 61-63 μm² on average) and were significantly larger than the spherulites (about 24-25 μm² on average) observed in formulations prepared by a continuous heating-cooling process.

In addition, crystals with plate structures were smaller in formulations prepared by a continuous heating-cooling process (about 12-13 μm² on average) compared to those prepared by a holding process (about 15-19 μm² on average). Among the crystals observed, spherulites (22-23%) and Tmh crystals (30-33%) occupied higher percentages of the area tested for formulations prepared by a continuous heating-cooling process and a holding process, respectively.

When formulations prepared by a continuous heating-cooling process were heated from 25° C. to 80° C., the structures that melted at the higher temperature (between 50-80° C.) were spherulites (FIG. 2D upper panel). Without being bound by any theory, as hydrogenated castor oil has the highest melting temperature amongst the excipients within the composition (83-88° C. in its pure form and about 60-70° C. in the formulation mixture, see Example 2), it is logical that these structures correspond to the hydrogenated castor oil. When formulations prepared by a holding process were heated from 25° C. to 80° C., the structures that melted at the higher temperatures (between 50-80° C.) were Tmh crystals (FIG. 2D lower panel). Without being bound by any theory, as hydrogenated castor oil has the highest melting temperature amongst the excipients within the composition and as all the other foam adjuvants and wax should have melted at about or lower than 70° C. based on their melting temperature (see Table 1), it is likely that these structures correspond to the hydrogenated castor oil. Analysis of the melting temperatures of each of the different components of the formulation (Example 2) showed that HCO was the only component with a melting point of above 50° C. (about 60-70° C.). Other components of the formulation (e.g. Docosanol, stearic acid, stearyl alcohol, beeswax), although having a melting point of above 50° C. in their pure state (Table 1), had a melting temperature below 50° C. when added to the formulation. Therefore, it follows that the structures observed when the formulation is heated to 50° C., i.e. spherulites in formulations prepared by a continuous heating-cooling process and Tmh crystals in formulations prepared by a holding process, correspond to HCO.

Formulations with 2% HCO showed a similar effect. When prepared by a continuous heating-cooling process, spherulites and small plates were formed and when prepared by a holding process, large plates and Tmh crystals were formed (FIG. 2E).

In order to simulate the effect of including a propellant in the foamable formulations, an equivalent weight of heptane was added to formulations with 1.2% HCO comprising MCH+ADP prepared by a holding process. Formulations were kept at 5° C. or 25° C. and tested after 15 days. Tmh crystals were observed in these formulations (FIG. 2F), indicating that the crystal fingerprint is retained even after dilution by heptane (simulating propellant dilution).

To test whether Tmh crystals were present in a foam expelled from a canister, foams kept at 40° C. comprising formulations with MCH+ADP and 1.2% HCO prepared by a continuous heating-cooling process or a holding process were visualized by a microscope. A small amount of foam was expelled from the canister and collapsed by storing for 10 minutes at 36° C. The collapsed foam was immediately cooled to 25° C. As can be seen in FIG. 2G, Tmh crystals could be observed in microphotographs of a foam formulation prepared by a holding process. These structures were not observed in a foam formulation prepared by a continuous heating-cooling process. These results indicate that the formulations prepared by a holding process with or without propellant, before or after release from the canister to form a foam, all have similar crystal fingerprints. Thus, the crystal fingerprint is maintained despite dilution as a result of the addition of propellant. The crystal fingerprint is also similarly maintained unchanged upon release of the foamable composition to form a foam, despite concentration and expansion of the formulation as the propellant dissipates.

As shown in FIG. 2H and Tables 11A-C, DSC analysis of formulations with 1.2% HCO comprising MCH+ADP kept at 5° C. showed different thermograms when the formulation was prepared by a continuous heating-cooling process (190319S) compared to a holding process (190505S). For example, TM4 was about 2.5° C. higher on Day 15 (68.5° C. vs. 66.0° C.) and Day 30 (68.6° C. vs. 66.1° C.) for a formulation prepared by a holding process.

When the same formulations were kept at 25° C. (FIG. 2I and Tables 11A-C), the formulation prepared by a continuous heating-cooling process changed over time: T_{M4} enthalpy was reduced and new transitions were observed such as T_{M3.1} and T_{M4.1}. On the other hand, a formulation prepared by a holding process remained stable over time; T_{M4} was about 3° C. higher from Day 0 through Day 30 for a formulation prepared by the holding process compared to that prepared by a continuous heating-cooling process (69.3° C. vs. 66.2 on Day 0; 69.2° C. vs. 66° C. on Day 15; 68.8° C. vs. 65.7° C. on Day 30).

When stored at 40° C. (FIG. 2J and Tables 11A-C), T_{M4} faded over time in a formulation prepared by a continuous heating-cooling process. On the contrary, T_{M4} remained stable from Day 0 through Day 30 in a formulation prepared by a holding process. The increase in temperature to 40° C. also resulted in the appearance of new transitions in the formulation prepared by a holding process. Thus, it may be possible that the changes observed in the continuous heating-cooling process are delayed in a formulation prepared by a holding process. In other words, new transitions observed in the formulation prepared by a continuous heating-cooling process at 25° C., were seen in a formulation prepared by a holding process only at 40° C. Without being bound by any theory it may be possible that the holding process “rescues” or holds back the formulation from forming the less stable structures corresponding to transitions T_{M3.1} and T_{M4.1} at the lower temperature (25° C.). Keeping formulations at high temperatures is often used to test formulation aging. Thus, these changes that appeared in the DSC only at a higher temperature for a formulation prepared by a holding process may indicate that structural changes may be delayed by the holding process.

The differences between the continuous heating-cooling process and the holding process were also evident in the placebo formulations with 1.2% HCO kept at 5° C., 25° C. and 40° C. where T_{M4} was 5.5-6.1° C. higher in formulations prepared by a holding process (FIG. 2K and Tables 11A-C). For example, on Day 15 T_{M4} was 71.1° C. for a formulation prepared by a holding process and kept at 25° C., compared to 65.4° C. for a formulation prepared by a continuous heating-cooling process. Thus, the unexpected structural changes and/or crystal fingerprint arising from the holding process are also applicable to carrier formulations without a therapeutic agent as well as to therapeutic compositions. Without being bound by theory, using a holding step to produce Tmh crystals in a formulation comprising HCO may prevent a reduction in Tₘ (compare the drop in Tₘ reported in Example 2 when the formulations are prepared without a holding step.)

Active and Placebo formulations with 1.2% or 2% HCO were prepared by either a holding process or a continuous heating-cooling process.

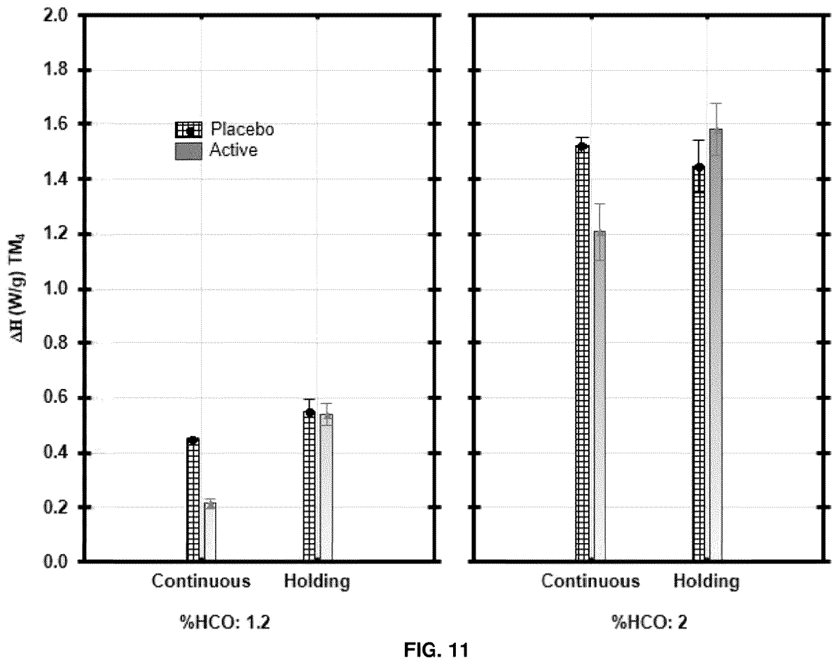

DSC analysis of these formulations showed that T_{M4} enthalpy was higher in all formulations prepared by a holding process compared to formulations prepared by a continuous heating-cooling process (except for placebo formulations with 2% HCO that had similar T_{M4} enthalpy) (FIG. 11). This increased enthalpy indicates the structures created in the holding process have stronger bonds, as the energy required to break these bonds is higher than that for the structures created in a continuous heating-cooling process.

Formulations comprising MCH+ADP with 1.2% HCO were prepared by a continuous heating-cooling process and a holding process, kept at 40° C. and analyzed on Day 0 by small angle X-ray scattering. As shown in FIG. 2L, the formulation prepared by a holding process had a higher intensity compared to the same formulation prepared by a continuous heating-cooling process. For example, at 2θ=0.85, the average intensity for the formulation prepared by a holding process (two replicates, upper lines) was 0.033 cm⁻¹ versus 0.014 cm⁻¹ for the formulation prepared by a continuous heating-cooling process (two replicates, lower lines). For measurements performed at 2θ=0.7-1.2 the average intensity for a formulation prepared by a holding process was 0.031 cm⁻¹ versus 0.014 cm⁻¹ for a formulation prepared by a continuous heating-cooling process. In other words, the average intensity of a formulation prepared by a holding process was more than 2 times higher than that of a formulation prepared by a continuous heating-cooling process. The higher intensity represents crystals of larger domains, i.e. having a larger crystalline body, and higher order, i.e. the molecular forces participating in the formation of the crystalline form are stronger.

Formulations comprising MCH+ADP with 1.2% HCO were prepared by a continuous heating-cooling process and a holding process, kept at 25° C. and analyzed on Day 0 by wide angle X-ray scattering. As can be seen in FIG. 2M, no differences were observed between the formulations prepared by the different processes. This may indicate that there was no change in the crystalline form, or that any change that did occur was subtle but undetectable by wide-angle X-ray crystallography.

Although there was no observed change in crystalline form, configuration, or lattice, as seen by wide angle X-ray crystallography, the holding process resulted in a significant change in crystal morphology and/or microstructure which provided a novel crystal fingerprint, as seen by small angle X-ray crystallography.

The results presented above indicate that changing to a holding process resulted in a change of microstructures within the formulation; spherulites were mainly formed by a continuous heating-cooling process whereas Tmh crystals and larger plates were formed by a holding process. The Tmh crystals had higher thermodynamic stability and a higher melting point compared to the spherulites formed by a continuous heating-cooling process.

The results indicate that the novel holding process described herein forms a unique Tmh crystal structure previously not described in the literature. Wax crystals are distributed throughout the composition. While larger crystal forms can be observed to cluster in certain areas of the composition, these clusters appear to be distributed uniformly throughout the formulation. The crystal forms, including their numbers, sizes and distribution etc., result in a unique crystal fingerprint. See e.g., Tables 11A to 11C below.

TABLE 11A

| Study | | | 3.7 | | | 3.9 | | | 3.10 | | |
|---|---|---|---|---|---|---|---|---|---|---|---|
| Formulation # | | | 190319S | | | 190505S | | | 190430S | | |
| HCO amount | | | 1.2% | | | 1.2% | | | 1.2% | | |
| API | | | MCH + ADP | | | MCH + ADP | | | ADP | | |
| Process | | | Continuous heating- | | | Holding process | | | Holding process | | |
| | | | cooling | | | | | | | | |
| Component | | | | | | | | | | | |
| Soybean oil | | | 50 | | | 50 | | | 50 | | |
| coconut oil | | | 23.6 | | | 23.6 | | | 23.6 | | |
| Light mineral oil* | | | 3.3 | | | 3.3 | | | 6.3 | | |
| Stearic acid | | | 3 | | | 3 | | | 3 | | |
| Docosanol | | | 1.1 | | | 1.1 | | | 1.1 | | |
| Hydrogenated | | | 1.2 | | | 1.2 | | | 1.2 | | |
| castor oil | | | | | | | | | | | |
| White wax | | | 2 | | | 2 | | | 2 | | |
| (beeswax) | | | | | | | | | | | |
| Stearyl alcohol | | | 1.5 | | | 1.5 | | | 1.5 | | |
| Cetostearyl | | | 3.5 | | | 3.5 | | | 3.5 | | |
| alcohol | | | | | | | | | | | |
| Myristyl alcohol | | | 2.5 | | | 2.5 | | | 2.5 | | |
| Cyclomethicone | | | 5 | | | 5 | | | 5 | | |
| Minocycline | | | 3 | | | 3 | | | 0 | | |
| HCl** | | | | | | | | | | | |
| Adapalene | | | 0.3 | | | 0.3 | | | 0.3 | | |
| TOTAL | | | 100 | | | 100 | | | 100 | | |
| | Tm/ | | | | | | | | | | |
| Test | Time | | T0 | T15 | T30 | T0 | T15 | T30 | T0 | T15 | T30 |
| Shakability | 5° | C. | NA | NA | S | S | S | S | S | S | S |
| | 25° | C. | NA | NA | M | S | S | S | S | S | S |
| | 40° | C. | NA | NA | N | S | M | M | S | M | M |
| DSC | 5° | C. | NA | 27.1 | 26.6 | NA | 27.1 | 26.8 | NA | 27.3 | 26.9 |
| TM2 | | | | 0.3 | 0.3 | | 0.1 | 0.1 | | 0.0 | 0.1 |
| | 25° | C. | 27.2 | 27.4 | 26.9 | 26.9 | 27.1 | 27.1 | 26.8 | 27.3 | 27.9 |
| | | | 0.1 | 0.1 | 0.1 | 0.1 | 0.1 | 0.1 | 0.2 | 0.2 | 0.1 |
| | 40° | C. | 26.6 | | | 26.5 | | | 26.6 | 27.3** | |
| | | | 0.01 | | | 0.1 | | | 0.2 | | |
| DSC | 5° | C. | NA | | | NA | | | NA | | |
| TM2.1 | 25° | C. | | | | | | | | | |
| | 40° | C. | 31.8 | 30.1 | 30.1 | 31.9 | 29.2 | 30.3 | 32.3 | 30.6** | |
| | | | 0.2 | 1.0 | 0.3 | 0.3 | 2.0 | 0.3 | 0.1 | | |
| DSC | 5° | C. | NA | | | NA | | | NA | | |
| TM3.1 | | | | | | | | | | | |
| | 25° | C. | | 35.8 | 35.6 | | | | | | |
| | | | | 0.1 | 0.4 | | | | | | |
| | 40° | C. | 40.0 | 39.5 | 39.7 | 39.9 | 38.3 | 38.8 | 39.3 | 39.0 | 38.4 |
| | | | 0.2 | 0.5 | 0.1 | 0.3 | 0.1 | 0.3 | 0.2 | 0.1 | 0.3 |
| DSC | 5° | C. | NA | 41.9 | 41.5 | NA | 42.4 | 42.1 | NA | 41.0 | 40.8 |
| TM3 | | | | 0.4 | 0.1 | | 0.1 | 0.1 | | 0.6 | 0.1 |
| | 25° | C. | 42.3 | 42.4 | 41.7 | 42.1 | 42.3 | 41.9 | 41.0 | 41.5 | 40.8 |
| | | | 0.3 | 0.4 | 0.0 | 0.2 | 0.7 | 0.2 | 0.2 | 0.1 | 0.1 |
| | 40° | C. | 43.2 | 44.1 | 43.9 | 43.5 | 43.8 | 44.1 | 42.8 | 44.1 | 43.9 |
| | | | 0.0 | 0.1 | 0.1 | 0.1 | 0.1 | 0.2 | 0.0 | 0.2 | 0.1 |
| DSC | 5° | C. | NA | | | NA | | | NA | | |
| TM4.2 | | | | | | | | | | | |
| | 25° | C. | | | | | | | | | |
| | 40° | C. | | | | | 53.7 | 56.1 | | 52.07** | 53.97* |
| | | | | | | | 0.1 | 0.5 | | | |
| DSC | 5° | C. | NA | | | NA | | | NA | 56.3 | 55.7 |
| TM4.1 | | | | | | | | | | 0.5 | 0.1 |
| | 25° | C. | 58.1 | 58.0 | 57.2 | | | | 55.9 | 56.8 | 56.2 |
| | | | 0.2 | 0.1 | 0.7 | | | | 0.5 | 0.0 | 0.0 |
| | 40° | C. | 56.6 | 59.3 | 59.7 | | | | 55.8 | 57.0 | 57.5 |
| | | | 0.3 | 0.7 | 0.3 | | | | 0.1 | 0.1 | 0.1 |
| DSC | 5° | C. | NA | 66.0 | 66.1 | NA | 68.5 | 68.6 | NA | 69.1 | 69.9 |
| TM4 | | | | 0.4 | 0.1 | | 0.4 | 0.2 | | 0.0 | 0.7 |
| | 25° | C. | 66.2 | 66.0 | 65.7 | 69.3 | 69.2 | 68.8 | 69.7 | 70.5 | 68.8 |
| | | | 0.1 | 0.1 | 0.0 | 0.9 | 0.2 | 0.5 | 0.8 | 0.4 | 1.7 |
| | 40° | C. | 66.1 | | | 69.4 | 68.4 | 68.6 | 69.9 | 69.3 | 69.2 |
| | | | 0.4 | | | 0.3 | 0.2 | 0.4 | 0.2 | 0.8 | 0.1 |
| Rheology | 5° | C. | NA | 124.1 | 325.3 | NA | 122.1 | 128.9 | NA | 58.5 | 52.5 |
| G′ Pa | | | | 38.6 | 21.5 | | 11.1 | 27.3 | | 7.7 | 6.1 |
| | 25° | C. | 87.4 | 134.6 | 237.2 | 58.9 | 346.4 | 434.2 | 57.9 | 99.8 | 253.4 |
| | | | 13.3 | 3.4 | 17.4 | 1.4 | 29.5 | 10.9 | 8.0 | 24.4 | 43.9 |
| | 40° | C. | 2879.1 | 11136.2 | 5664.8 | 12372.5 | 7884.6 | 5933.6 | 8018.4 | 8435.0 | 6926.2 |
| | | | 1300.3 | 1674.0 | 387.5 | 482.2 | 1891.3 | 584.7 | 291.8 | 1493.8 | 317.3 |
| Rheology | 5° | C. | NA | 26.5 | 26.8 | NA | 26.4 | 26.7 | NA | 25.9 | 26.2 |
| Flow | | | | 0.1 | 0.3 | | 0.8 | 1.1 | | 0.4 | 0.5 |
| Point, ° C. | 25° | C. | 27.3 | 32.7 | 33.2 | 24.9 | 34.6 | 36.3 | 27.35 | 30.6 | 34.6 |
| | | | 0.6 | 2.1 | 0.2 | 0.5 | 0.5 | 0.2 | 0.0 | 0.9 | 0.8 |
| | 40° | C. | 33.0 | 59.6 | 59.7 | 32.2 | 53.65 | 54.85 | 33.3 | 56.5 | 56.3 |
| | | | 0.0 | 0.6 | 0.6 | 0.0 | 0.5 | 1.3 | 0.4 | 0.1 | 0.4 |
| Rheology | 5° | C. | NA | 287.3 | 364.4 | NA | 244.5* | 291.6* | NA | 249.1* | 256.1* |
| Viscosity, | | | | 11.2 | 4.3 | | | | | | |
| CP | 25° | C. | 258.4 | 331.9 | 362.7 | 251.1* | NA | 368.9* | 191.9* | 271.2* | 256.1* |
| | | | 20.8 | 20.2 | 8.0 | | | | | | |
| | 40° | C. | NA | NA | NA | NA | NA | NA | NA | NA | NA |

*Only one replicate was measured
**Two replicates were measured but parameter was present in only one replicate

TABLE 11B

| Study | | | 3.8 | | | 3.11 | | | 3.2 | | |
|---|---|---|---|---|---|---|---|---|---|---|---|
| Formulation # | | | PC1.2 | | | PH1.2 | | | 190313N | | |
| HCO amount | | | 1.2% | | | 1.2% | | | 2% | | |
| API | | | Placebo | | | Placebo | | | MCH + ADP | | |
| Process | | | Continuous heating- | | | Holding | | | Continuous heating- | | |
| | | | cooling | | | | | | cooling | | |
| Component | | | | | | | | | | | |
| Soybean oil | | | 50 | | | 50 | | | 50 | | |
| coconut oil | | | 23.6 | | | 23.6 | | | 23.60 | | |
| Light mineral oil* | | | 6.6 | | | 6.6 | | | 2.5 | | |
| Stearic acid | | | 3 | | | 3 | | | 3.00 | | |
| Docosanol | | | 1.1 | | | 1.1 | | | 1.1 | | |
| Hydrogenated | | | 1.2 | | | 1.2 | | | 2 | | |
| castor oil | | | | | | | | | | | |
| White wax | | | 2 | | | 2 | | | 2 | | |
| (beeswax) | | | | | | | | | | | |
| Stearyl alcohol | | | 1.5 | | | 1.5 | | | 1.5 | | |
| Cetostearyl | | | 3.5 | | | 3.5 | | | 3.5 | | |
| alcohol | | | | | | | | | | | |
| Myristyl alcohol | | | 2.5 | | | 2.5 | | | 2.5 | | |
| Cyclomethicone | | | 5 | | | 5 | | | 5 | | |
| Minocycline | | | 0 | | | 0 | | | 3 | | |
| HCl** | | | | | | | | | | | |
| Adapalene | | | 0 | | | 0 | | | 0.30 | | |
| TOTAL | | | 100 | | | 100 | | | 100 | | |
| | Tm/ | | | | | | | | | | |
| Test | Time | | T0 | T15 | T30 | T0 | T15 | T30 | T0 | T15 | T30 |
| Shakability | 5° | C. | NA | NA | NA | NA | NA | NA | NA | NA | S |
| | 25° | C. | NA | NA | NA | NA | NA | NA | NA | NA | N |
| | 40° | C. | NA | NA | NA | NA | NA | NA | NA | NA | N |
| DSC | 5° | C. | NA | 27.1 | 27.4 | NA | 26.7 | 27.0 | NA | 27.6 | 26.7 |
| TM2 | | | | 0.2 | 0.0 | | 0.1 | 0.3 | | 0.5 | 0.2 |
| | 25° | C. | 27.7 | 26.8 | 26.7 | 26.7 | 26.7 | 26.5 | 27.5 | 27.5 | 28.3 |
| | | | 0.0 | 0.1 | 0.2 | 0.2 | 0.1 | 0.0 | 0.4 | 0.2 | 0.2 |
| | 40° | C. | 27.5 | 26.5 | | 26.9 | 26.5 | | 26.6 | | |
| | | | 0.1 | 0.1 | | 0.0 | 0.1 | | 0.2 | | |
| DSC | 5° | C. | NA | | | NA | | | NA | | |
| TM2.1 | 25° | C. | | | | | | | | | |
| | 40° | C. | | | 31.6 | 33.0 | 34.0 | 34.5 | | 27.9 | 29.6 |
| | | | | | 0.5 | 0.2 | 0.3 | 0.2 | | 0.7 | 0.1 |
| DSC | 5° | C. | NA | | | NA | 35.2 | 35.0 | NA | | |
| TM3.1 | | | | | | | 0.3 | 0.1 | | | |
| | 25° | C. | | 37.9 | 40.6 | 35.4 | 36.8 | | | | |
| | | | | 0.3 | 0.6 | 0.0 | 0.1 | | | | |
| | 40° | C. | | 36.2 | | | | | 39.8 | 39.5 | 39.3 |
| | | | | 0.0 | | | | | 0.1 | 0.3 | 0.1 |
| DSC | 5° | C. | NA | 43.0 | 43.8 | NA | 42.4 | 43.0 | NA | 41.5 | 43.1 |
| TM3 | | | | 0.2 | 0.3 | | 0.2 | 0.1 | | 0.1 | 0.7 |
| | 25° | C. | 43.5 | 43.8 | 43.1 | 42.8 | 43.1 | 41.1 | 42.2 | 42.6 | 42.1 |
| | | | 0.1 | 0.3 | 0.1 | 0.2 | 0.1 | 1.2 | 0.2 | 0.3 | 0.3 |
| | 40° | C. | 43.9 | 46.1 | 44.1 | 43.4 | 43.1 | 43.7 | 43.4 | 44.1 | 44.1 |
| | | | 0.1 | 0.1 | 0.5 | 0.1 | 0.2 | 0.0 | 0.0 | 0.0 | 0.1 |
| DSC | 5° | C. | NA | | | NA | 51.2 | | NA | | |
| TM4.2 | | | | | | | 0.1 | | | | |
| | 25° | C. | | | | 50.7 | | | | | |
| | | | | | | 0.5 | | | | | |
| | 40° | C. | | | | 51.1 | | | | | |
| | | | | | | 0.0 | | | | | |
| DSC | 5° | C. | NA | | | NA | | 58.7 | NA | | |
| TM4.1 | | | | | | | | 0.3 | | | |
| | 25° | C. | | | | 58.4 | 59.0 | 58.9 | | | |
| | | | | | | 0.3 | 0.5 | 0.4 | | | |
| | 40° | C. | | 59.0 | | 58.5 | 58.7 | 58.6 | | 58.6 | 62.0 |
| | | | | 0.3 | | 0.0 | 0.8 | 0.1 | | 0.2 | 0.0 |
| DSC | 5° | C. | NA | 65.8 | 65.9 | NA | 71.5 | 70.6 | NA | 67.6 | 68.0 |
| TM4 | | | | 0.2 | 0.3 | | 0.2 | 0.3 | | 0.1 | 0.1 |
| | 25° | C. | 65.6 | 65.4 | 66.0 | 71.1 | 71.0 | 70.0 | 68.4 | 68.4 | 68.5 |
| | | | 0.2 | 0.0 | 0.0 | 0.1 | 0.3 | 1.0 | 0.1 | 0.3 | 0.3 |
| | 40° | C. | 65.4 | 64.7 | 65.6 | 71.5 | 70.8 | 70.6 | 68.1 | 68.0 | 67.2 |
| | | | 0.1 | 0.1 | 0.2 | 0.1 | 0.0 | 0.0 | 0.4 | 0.3 | 0.1 |
| Rheology | 5° | C. | NA | 146.3 | 302.1 | NA | 293.6 | 225.3 | NA | 89.1 | 450.1 |
| G′ Pa | | | | 7.6 | 18.2 | | 102.5 | 39.2 | | 3.8 | 27.0 |
| | 25° | C. | 159.3 | 902.2 | 1585.2 | 237.3 | 389.5 | 397.1 | 127.5 | 285.7 | 560.1 |
| | | | 28.9 | 19.7 | 405.1 | 5.2 | 101.6 | 5.9 | 3.8 | 61.7 | 28.7 |
| | 40° | C. | 8179.0 | 6300.4 | 7268.7 | 7864.1 | 9132.0 | 7060.3 | 8416.8 | 14139.6 | 19584.3 |
| | | | 691.0 | 2090.0 | 3120.47 | 554.8 | 2226.9 | 554.8 | 2835.5 | 2336.3 | 4209.0 |
| Rheology | 5° | C. | NA | 28.5 | 31.6 | NA | 30.0 | 29.0 | NA | 26.9 | 27.4 |
| Flow | | | | 0.2 | 1.9 | | 0.0 | 0.1 | | 1.1 | 0.2 |
| Point, ° C. | 25° | C. | 28.6 | 41.4 | 48.0 | 38.0 | 35.15 | 36.7 | 27.6 | 32.2 | 37.0 |
| | | | 0.3 | 0.1 | 0.7 | 3.5 | 1.6 | 0.5 | 0.7 | 1.6 | 0.8 |
| | 40° | C. | 35.55 | 62.25 | 62.95 | 38.5 | 57.5 | 58.8 | 34.2 | 61.9 | 64.0 |
| | | | 3.0 | 0.6 | 1.5 | 7.6 | 0.3 | 0.3 | 1.7 | 1.1 | 0.8 |
| Rheology | 5° | C. | NA | 259.6 | 172.6 | NA | 193.8 | 241.6 | NA | 388.5 | 503.3 |
| Viscosity, | | | | 76.4 | 94.8 | | 45.3 | 152.5 | | 42.9 | 26.9 |
| cP | 25° | C. | 174.7 | 306.6 | 308.9 | 261.0 | 311.6 | 409.6 | 412.0 | 451.4 | 506.7 |
| | | | 17.3 | 11.3 | 89.7 | 12.9 | 18.2 | 1.0 | 108.1 | 17.7 | 12.0 |
| | 40° | C. | NA | NA | NA | NA | NA | NA | NA | NA | NA |

TABLE 11C

| Study | | | 3.4 | | | 3.3 | | | 3.5 | | |
|---|---|---|---|---|---|---|---|---|---|---|---|
| Formulation # | | | 190430R | | | PC2 | | | PH2 | | |
| HCO amount | | | 2% | | | 2% | | | 2% | | |
| API | | | MCH + ADP | | | Placebo | | | Placebo | | |
| Process | | | Holding | | | Continuous heating- | | | Holding | | |
| | | | | | | cooling | | | | | |
| Component | | | | | | | | | | | |
| Soybean oil | | | 50 | | | 50 | | | 50 | | |
| coconut oil | | | 23.6 | | | 23.6 | | | 23.6 | | |
| Light mineral | | | 2.5 | | | 5.8 | | | 5.8 | | |
| oil* | | | | | | | | | | | |
| Stearic acid | | | 3 | | | 3 | | | 3 | | |
| Docosanol | | | 1.1 | | | 1.1 | | | 1.1 | | |
| Hydrogenated | | | 2 | | | 2 | | | 2 | | |
| castor oil | | | | | | | | | | | |
| White wax | | | 2 | | | 2 | | | 2 | | |
| (beeswax) | | | | | | | | | | | |
| Stearyl alcohol | | | 1.5 | | | 1.5 | | | 1.5 | | |
| Cetostearyl | | | 3.5 | | | 3.5 | | | 3.5 | | |
| alcohol | | | | | | | | | | | |
| Myristyl alcohol | | | 2.5 | | | 2.5 | | | 2.5 | | |
| Cyclomethicone | | | 5 | | | 5 | | | 5 | | |
| Minocycline | | | 3 | | | 0 | | | 0 | | |
| HCl** | | | | | | | | | | | |
| Adapalene | | | 0.3 | | | 0 | | | 0 | | |
| TOTAL | | | 100 | | | 100 | | | 100 | | |
| | Tm/ | | | | | | | | | | |
| Test | Time | | T0 | T15 | T30 | T0 | T15 | T30 | T0 | T15 | T30 |
| Shakability | 5° | C. | S | S | S | NA | NA | NA | NA | NA | NA |
| | 25° | C. | S | S | S | NA | NA | NA | NA | NA | NA |
| | 40° | C. | S | M | N | NA | NA | NA | NA | NA | NA |
| DSC | 5° | C. | NA | 27.4 | 27.2 | NA | 26.9 | 27.1 | NA | 26.8 | 27.0 |
| TM2 | | | | 0.0 | 0.6 | | 0.3 | 0.1 | | 0.7 | 0.1 |
| | 25° | C. | 27.2 | 27.3 | 27.4 | 27.1 | 26.7 | 27.9 | 26.9 | 26.6 | 26.5 |
| | | | 0.7 | 0.3 | 0.4 | 1.0 | 0.1 | 0.8 | 0.1 | 0.2 | 0.2 |
| | 40° | C. | 26.7 | 27.3 | | 27.0 | 26.7 | | 27.4 | 26.6 | |
| | | | 0.1 | 0.2 | | 0.7 | 0.1 | | 0.2 | 0.2 | |
| DSC | 5° | C. | NA | | | NA | | | NA | 29.7 | 31.3 |
| TM2.1 | | | | | | | | | | 0.0 | 0.0 |
| | 25° | C. | | | | | | | 30.8 | | |
| | | | | | | | | | 0.6 | | |
| | 40° | C. | | | 29.7 | | | | | | |
| | | | | | 0.6 | | | | | | |
| DSC | 5° | C. | NA | | | NA | | | NA | | |
| TM3.1 | | | | | | | | | | | |
| | 25° | C. | | | | | | 38.5 | | | |
| | | | | | | | | 0.6 | | | |
| | 40° | C. | 39.7 | 38.0 | 37.7 | | 41.5 | NA | 39.4 | 34.3 | |
| | | | 0.3 | 0.6 | 0.4 | | 0.4 | | 0.1 | 0.2 | |
| DSC | 5° | C. | NA | 42.6 | 41.4 | NA | 43.7 | 43.4 | NA | 43.1 | 42.0 |
| TM3 | | | | 0.0 | 0.2 | | 0.1 | 0.1 | | 0.1 | 0.1 |
| | 25° | C. | 41.9 | 41.4 | 41.6 | 43.3 | 43.7 | 43.6 | 43.2 | 42.4 | 41.8 |
| | | | 0.3 | 0.5 | 0.1 | 0.1 | 0.1 | 0.1 | 0.2 | 0.0 | 0.0 |
| | 40° | C. | 43.5 | 43.7 | 43.5 | 43.4 | 45.7 | 43.9 | 43.4 | 44.1 | 44.1 |
| | | | 0.0 | 0.1 | 0.2 | 0.0 | 0.1 | 0.1 | 0.1 | 0.4 | 0.0 |
| DSC | 5° | C. | NA | | | NA | 56.0 | 55.2 | NA | | |
| TM4.2 | | | | | | | 0.4 | 0.4 | | | |
| | 25° | C. | | | | 55.3 | 56.0 | 56.0 | | | |
| | | | | | | 0.4 | 0.2 | 0.2 | | | |
| | 40° | C. | | | | 55.6 | | | | | |
| | | | | | | 0.6 | | | | | |
| DSC | 5° | C. | NA | | | NA | | | NA | | |
| TM4.1 | | | | | | | | | | | |
| | 25° | C. | | | | | | | | | |
| | 40° | C. | | 60.6 | 62.7 | | 60.2 | | | 60.7 | 61.2 |
| | | | | 0.1 | 0.3 | | 0.5 | | | 0.3 | 1.7 |
| DSC | 5° | C. | NA | 68.6 | 67.7 | NA | 68.1 | 68.1 | NA | 72.5 | 71.7 |
| TM4 | | | | 0.14 | 0.35 | | 0.5 | 0.1 | | 0.3 | 0.0 |
| | 25° | C. | 68.2 | 68.4 | 68.2 | 68.3 | 68.5 | 68.2 | 72.4 | 72.5 | 72.5 |
| | | | 0.28 | 0.21 | 0.49 | 0.2 | 0.4 | 0.1 | 0.3 | 0.3 | 0.0 |
| | 40° | C. | 67.9 | 67.3 | 66.3 | 68.0 | 66.4 | 67.1 | 71.8 | 71.8 | 71.7 |
| | | | 0.21 | 0.28 | 0.14 | 0.2 | 0.2 | 0.3 | 1.3 | 0.1 | 0.0 |
| Rheology | 5° | C. | NA | 134.0 | 247.3 | NA | 291.6 | 114.5 | NA | 425.0 | 318.2 |
| G′ Pa | | | | 21.8 | 17.8 | | 5.9 | 4.8 | | 61.4 | 78.3 |
| | 25° | C. | 280.8 | 268.5 | 366.7 | 100.9 | 693.8 | 1582.0 | 310.5 | 1132.7 | 1199.9 |
| | | | 75.8 | 20.3 | 20.6 | 34.0 | 131.8 | 492.7 | 56.9 | 247.2 | 81.3 |
| | 40° | C. | 16381.0 | 5918.5 | 6294.7 | 11191.0 | 6910.1 | 8417.7 | 19619.7 | 15667.7 | 21996.3 |
| | | | 204.1 | 1857.2 | 580.8 | 623.2 | 360.9 | 3033.5 | 2171.5 | 3803.2 | 153.2 |
| Rheology | 5° | C. | NA | 26.1 | 27.5 | NA | 30.8 | 29.1 | NA | 28.9 | 29.0 |
| Flow | | | | 0.1 | 0.1 | | 1.1 | 0.0 | | 0.9 | 0.3 |
| Point, ° C. | 25° | C. | 27.1 | 32.6 | 35.7 | 28.8 | 40.1 | 46.5 | 29.7 | 38.6 | 39.9 |
| | | | 0.9 | 0.3 | 2.6 | 0.8 | 0.1 | 3.3 | 0.8 | 0.8 | 0.5 |
| | 40° | C. | 33.3 | 43.7 | 46.8 | 38.7 | 63.5 | 66.3 | 37.8 | 65.0 | 65.1 |
| | | | 0.4 | 1.2 | 2.4 | 0.4 | 0.3 | 1.4 | 1.6 | 0.1 | 0.1 |
| Rheology | 5° | C. | NA | 380.0 | 401.8 | NA | 374.6 | 213.0 | NA | 234.3 | 440.7 |
| Viscosity, | | | | 0.0 | 0.0 | | 25.1 | 44.8 | | 35.9 | 1.4 |
| cP | 25° | C. | 303.6 | 416.3 | 472.5 | 268.3 | 298.6 | 304.6 | 309.4 | 487.0 | 529.9 |
| | | | 0.0 | 0.0 | 0.0 | 12.5 | 4.8 | 33.7 | 37.7 | 95.9 | 32.1 |
| | 40° | C. | NA | NA | NA | NA | NA | NA | NA | NA | NA |

*The amount of light mineral oil in the formulation is adjusted based on the amount of minocycline hydrochloride and/or adapalene.
**The amount of minocycline hydrochloride in the formulation is adjusted on the potency of the minocycline hydrochloride.

### Formulation Flow Point Study

Flow point was measured in a formulation comprising 3% minocycline and 0.3% adapalene, manufactured either using a continuous process, or using a holding process of 4 hours at 54° C. The measurements were performed at TO, and after 15 days and 30 days of storage at 40° C. (FIG. 2N).

The flow point of samples measured at TO was about 33° C. for formulation made with continuous or holding process. For samples stored at 15 days or 30 days at 40° C., an increase in the flow point was noticed. See Table 11A. Without being bound by theory, the tridimensional network responsible for the product structure may undergo changes at 40° C., with an increase in wax crystal-crystal interactions making a more resistant network. For samples stored at 15 days or 30 days at 40° C., the samples manufactured using a holding process had a lower flow point than with a continuous process. Without being bound by theory, the high-melting TMH crystals generated in the holding process may be more resistant to temperature changes, and less prone to form wax crystal-crystal interactions, resulting in a more flowable/shakable and less resistant network, and a lower flow point than that observed with the spherulite crystals generated in the continuous process.

### Example 4

### Effect on Sebum Softening

Mixtures of sebum with the tested formulations (Tables 12a, 12b): A. Formulation comprising MCH+ADP prepared by a holding process (MAH), B. Formulation comprising MCH prepared by a continuous heating-cooling process (MCO) and C. Oil-in-water emulsion (OIWE) were prepared by the process described in Example 1A and analyzed by DSC.

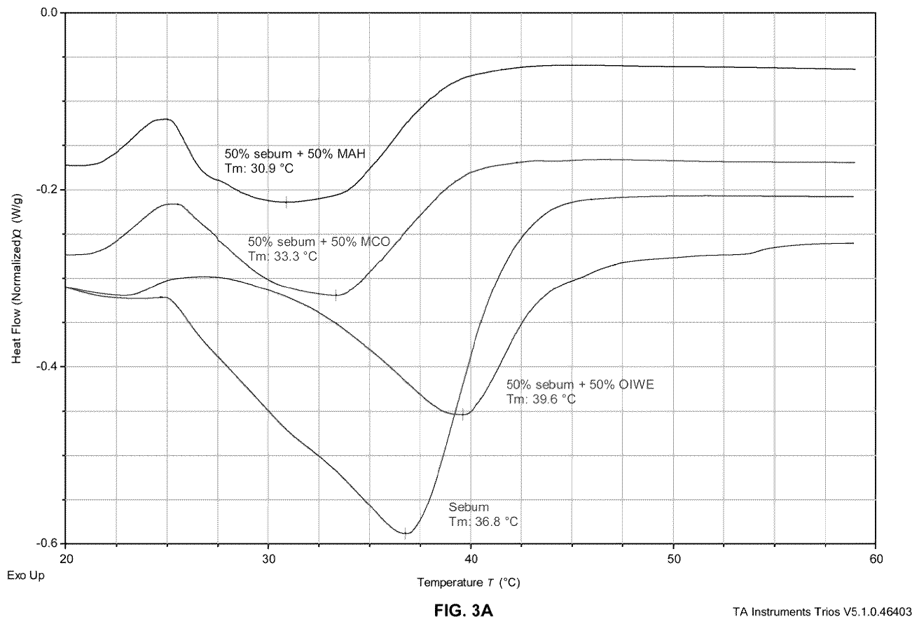

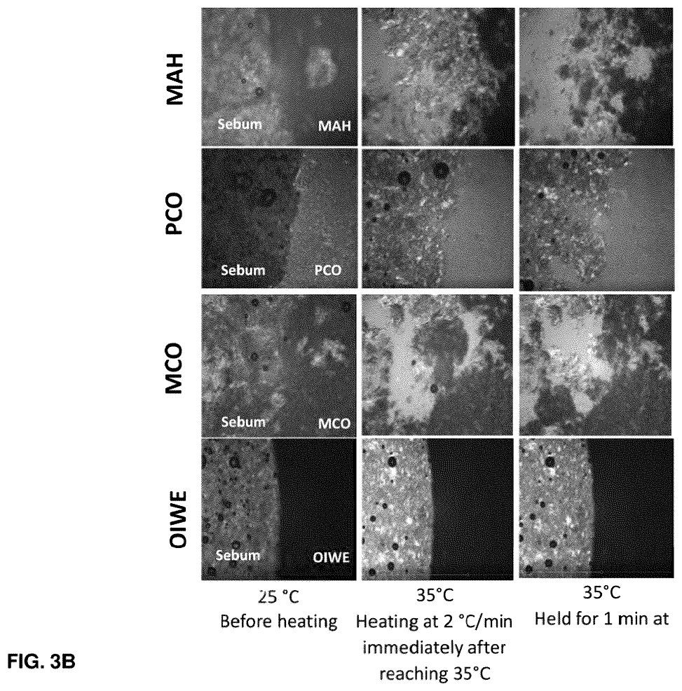

Table 12a and FIG. 3A provide the melting temperature of the sebum in the evaluated samples (determined here as minimum temperature of the melting endotherm and shown as Tm in Tables 12a, and 12b and in FIG. 3A). As can be seen, the Tm value of pure sebum was different from that of mixtures of sebum with the tested formulations (MAH, MCO, and OIWE).

A. The Tm value (FIG. 3A) of the endotherm for the sebum mixture with formulation MAH was 30.9° C., which was approximately 6° C. lower than that for the pure sebum (36.8° C.).

B. The Tm value of the endotherm for the sebum mixture with formulation MCO was 33.3° C., which was 3.5° C. lower than that for the pure sebum (36.8° C.).

C. On the contrary, for the mixture of sebum with an oil-in-water-emulsion (OIWE), the Tm value of the endotherm was at 39.6° C., which was approximately 3° C. higher than that for pure sebum.

A placebo formulation (PCO) with a composition equivalent to MCO, but with Minocycline HCl replaced by an equivalent weight of light mineral oil was also tested (Tables 12A-B).

Light microscopy was used to visualize the miscibility of the tested formulations (MAH, MCO, PCO (i.e. a placebo formulation), and OIWE) with sebum.

As shown in FIG. 3B, for all samples kept at 25° C., sebum (left) and the examined formulation (right) were separated and the border between them was clearly visible.

Before heating to 35° C., MAH and sebum showed extensive penetration into each other. The border between the sebum and formulation MAH was not visible and the sebum appears to flow.

After the PCO sample was heated at 2° C./min to 35° C., the low melting components of both sebum and PCO melted (became transparent), became intermixed and the border between sebum and PCO became blurred. After the mixture was held at 35° C. for 1 minute, the sebum and PCO appeared to be fully mixed with each other. A similar phenomenon was observed when MCH was present (i.e. in a sample of sebum and MCO).

In contrast, the border between sebum and OIWE remained intact even after heating to 35° C.. The melting of low-melting components of sebum could be observed, but even then, they remained in the sebum part of the sample, indicating a lack of miscibility between sebum and OIWE.

Therefore, microscopic evaluation of the mixtures of the tested formulations with sebum confirmed observations made by DSC that sebum, when mixed with MAH, prepared by a holding process, significantly reduced the melting temperature of the sample. When MCO, prepared by continuous heating-cooling process, was mixed with sebum it also resulted in a reduced melting temperature of the mixture, but to a lesser extent than that observed for formulation MAH prepared by a holding process.

TABLE 12a

| | | |
|---|---|---|
| Formulation | Minocycline | Minocycline formulation; |
| | and adapalene formulation; | Continuous heating- |
| | holding process (MAH) | cooling process (MCO) |
| HCO amount | 1.2% | 2% |
| API | MCH+ADP | MCH |
| Process | Holding | Continuous heating- |
| Component | | cooling |
| Soybean oil | 50 | 50 |
| Coconut oil | 23.6 | 23.6 |
| Light mineral | 3.3 | 1.8 |
| oil* | | |
| Stearic acid | 3 | 3 |
| Docosanol | 1.1 | 1.1 |
| Hydrogenated | 1.2 | 2 |
| castor oil | | |
| White wax | 2 | 2 |
| (beeswax) | | |
| Stearyl alcohol | 1.5 | 1.5 |
| Cetostearyl | 3.5 | 3.5 |
| alcohol | | |
| Myristyl alcohol | 2.5 | 2.5 |
| Cyclomethicone | 5 | 5 |
| Minocycline | 3 | 4 |
| HCl** | | |
| Adapalene | 0.3 | 0 |
| TOTAL | 100 | 100 |
| Propellant AP- | 0 | 12 |
| 70 | | |
| Test | | |
| Sebum melting | 30.9 | 33.3 |
| temperature | | |
| (Tm) | | |

TABLE 12b

| Model oil-in-water emulsion | | Artificial Sebum (Pickering Labs, | |
|---|---|---|---|
| formulation (OIWE) | | Product code 1700-0700) | |
| | Quantitative | | Quantitative |
| | Composition | | Composition |
| Component | (% w/w) | Component | (% w/w) |
| Caprylic/Capric | 8.00 | Palmitic acid | 10 |
| Triglyceride | | | |
| Stearic acid | 1.00 | Stearic acid | 5 |
| Ceteareth-20 | 3.30 | Coconut oil | 15 |
| Glyceryl monostearate | 1.70 | Paraffin wax | 10 |
| Hydroxypropyl | 0.35 | Synthetic | 15 |
| methylcellulose | | spermacetti | |
| Xanthan gum | 0.35 | Olive oil | 20 |
| Water | 82.25 | Squalene | 5 |
| Citric acid | 0.05 | Cholesterol | 5 |
| Glycerin | 3.00 | Oleic acid | 10 |
| Total | 100.00 | Linoleic acid | 5 |
| | | Total | 100 |
| Sebum melting | 39.6 | Sebum melting | 36.8 |
| temperature (Tm) | | temperature (Tm) | |

*The amount of light mineral oil in the formulation is adjusted based on the amount of minocycline hydrochloride and/or adapalene.
**The amount of minocycline hydrochloride in the formulation is adjusted by the potency of the minocycline hydrochloride.

### Example 5

Effect on Fluidity: Formulations with 2% or 1.2% HCO Prepared by a Continuous Heating-Cooling Process or a Holding Process Visualized in Glass Bottles.

This example compares fluidity/blockage of formulations prepared by different processes.

Different formulations as described in Table 5 were prepared and evaluated in transparent glass bottles. Formulation were kept at 5° C., 25° C. or 40° C. and pictures were taken at Day 15 and Day 30. Vials were tilted to test the fluidity of the formulations.

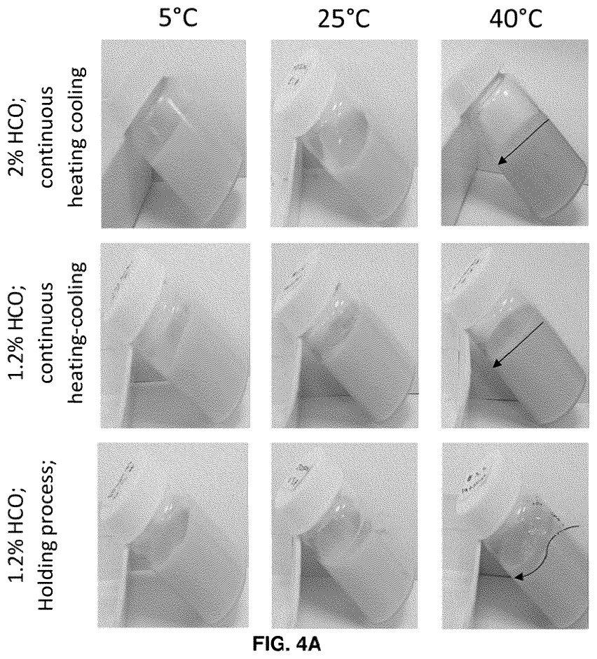

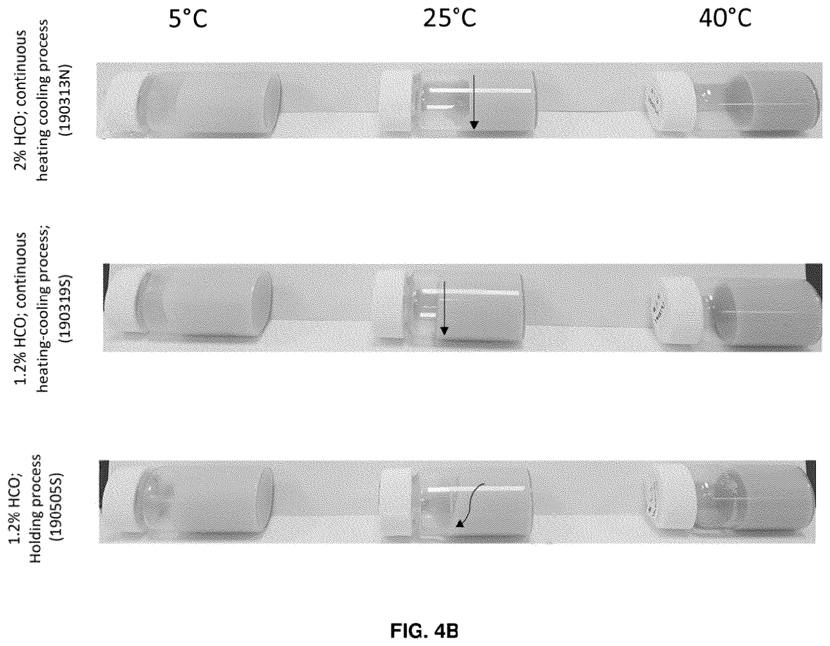

As shown in FIG. 4A, formulations prepared by a continuous heating-cooling process (190313N, 190319S) were more rigid and less fluid at 25° C. or 40° C. on Day 15 compared to a formulation prepared by a holding process (190505S). A similar phenomenon was observed on Day 30 (FIG. 4B). Without being bound by theory, the improved fluidity of the formulation prepared by a holding process may contribute to the improved shakability of this formulation compared to formulations prepared by a continuous heating-cooling process that were less fluid and less shakable.

TABLE 13

| | | | |
|---|---|---|---|
| Study | 1.2 | 1.5 | 3.9 |
| Formulation # | 190313N | 190319S | 190505S |
| HCO amount | 2% | 1.2% | 1.2% |
| API | MCH + ADP | MCH + ADP | MCH + ADP |
| Process | Continuous | Continuous | Holding |
| | heating- | heating- | |
| | cooling | cooling | |
| Component | | | |
| Soybean oil | 50 | 50 | 50 |
| coconut oil | 23.60 | 23.6 | 23.6 |
| Light mineral oil* | 2.5 | 3.3 | 3.3 |
| Stearic acid | 3.00 | 3 | 3 |
| Docosanol | 1.1 | 1.1 | 1.1 |
| Hydrogenated | 2 | 1.2 | 1.2 |
| castor oil | | | |
| White wax | 2 | 2 | 2 |
| (beeswax) | | | |
| Stearyl alcohol | 1.5 | 1.5 | 1.5 |
| Cetostearyl | 3.5 | 3.5 | 3.5 |
| alcohol | | | |
| Myristyl alcohol | 2.5 | 2.5 | 2.5 |
| Cyclomethicone | 5 | 5 | 5 |
| Minocycline | 3 | 3 | 3 |
| HCl** | | | |
| Adapalene | 0.30 | 0.3 | 0.3 |
| TOTAL | 100 | 100 | 100 |

*The amount of light mineral oil in the formulation is adjusted based on the amount of minocycline hydrochloride and/or adapalene.
**The amount of minocycline hydrochloride in the formulation is adjusted by the potency of the minocycline hydrochloride.

### Effect on Shakability

As shown in Tables 11A-C, shakability was improved in combination formulations (MCH+ADP) prepared by a holding process compared to formulations prepared by a continuous heating-cooling process. For example, a combination formulation comprising 2% HCO kept at 25° C. for 30 days was observed to be non-shakable when prepared by a continuous heating-cooling process (190313N) and fully shakable when prepared by a holding process (190430R). Similarly, a combination formulation comprising 1.2% HCO kept at 25° C. for 30 days was moderately-shakable when prepared by a continuous heating-cooling process (190319S) and fully shakable when prepared by a holding process (190505S). The same formulations, when kept at 40° C. for 30 days were non-shakable when prepared by a continuous heating-cooling process and moderately shakable when prepared by a holding process. Without being bound by any theory it is possible that the presence of Tmh crystals produced by the holding process and the reduced amount of HCO (from 2% to 1.2%) each contributed to the improved shakability.

Formulations prepared by a holding process that had superior shakability (See Tables 11A-C) surprisingly comprised nonuniform, large and stable crystals that were not observed in formulations prepared by a continuous heating-cooling process. This was unexpected as wide-angle X-ray data showed no apparent change in polymorph (Example 3). Without being bound by any theory, the stable structures and crystal fingerprint formed by the holding process may be less available to interact with each other and/or with other components of the formulation. This may improve the flowability of the formulation, resulting in a fully shakable formulation.

### Example 6

Shakability and Collapse Time Data Through 6 Months Period in a Continuous Heating-Cooling Process Vs. Holding Process.

This example compares shakability of formulations prepared by different processes.

Shakability and collapse time of formulations containing MCH and ADP with 1.2% or 2% HCO prepared by a continuous heating-cooling process, a holding process or an alternative holding process (Table 14) were measured for six months.

TABLE 14

| Formulation # | 171018N | 180225S | 180806S | 180304S |
|---|---|---|---|---|
| HCO amount | 2% | 1.2% | 1.2% | 1.2% |
| API | MCH + ADP | MCH + ADP | MCH + ADP | MCH + ADP |
| Process | Continuous | Continuous | Holding | Alternative |
| | heating- | heating- | | holding |
| | cooling # | cooling # | | process |
| | | | | (30 min at |
| | | | | 55-58° |
| | | | | C.) ## |
| Component | 171018N | 180225S | 180806S | 180304S |
| Soybean oil | 50 | 50 | 50 | 50 |
| coconut oil | 23.60 | 23.6 | 23.6 | 23.6 |
| Light mineral oil* | 3.3 | 2.5 | 2.5 | 2.5 |
| Stearic acid | 3 | 3 | 3 | 3 |
| Docosanol | 1.1 | 1.1 | 1.1 | 1.1 |
| Hydrogenated castor oil | 2 | 1.2 | 1.2 | 1.2 |
| White wax (beeswax) | 2 | 2 | 2 | 2 |
| Stearyl alcohol | 1.5 | 1.5 | 1.5 | 1.5 |
| Cetostearyl alcohol | 3.5 | 3.5 | 3.5 | 3.5 |
| Myristyl alcohol | 2.5 | 2.5 | 2.5 | 2.5 |
| Cyclomethicone | 5 | 5 | 5 | 5 |
| Minocycline HCl** | 3 | 3 | 3 | 3 |
| Adapalene | 0.30 | 0.3 | 0.3 | 0.3 |
| TOTAL | 100 | 100 | 100 | 100 |

*The amount of light mineral oil in the formulation is adjusted based on the amount of minocycline hydrochloride and/or adapalene.
**The amount of minocycline hydrochloride in the formulation is adjusted by the potency of the minocycline hydrochloride.
# For formulations 171018N and 180225S, continuous heating and cooling process was performed as described in Example 1A. Addition of retinoids was performed at about 35-40° C. and there was no overnight mixing step for up to 24 hours.
## Alternative holding process for 180304S formulation was performed as follows:
Step 1: Some of the hydrophobic solvents and solid compounds such as fatty alcohols, fatty acids and waxes (for e.g. soybean oil, HCO, beeswax) were mixed and heated to a temperature sufficient to achieve complete melting.
Step 2: The formulation was cooled down to about 55-58° C. and was mixed under this temperature for a period of 30 min.
Step 3: The formulation was heated to about 65° C. and the rest of the hydrophobic solvents and solid compounds was added and mixed until complete melting. The process then continued as described in Example 1b Step 3 onwards.

Shakability of the formulations tested is presented in Table 15 and foam quality, collapse time, and time to FG of the formulations are shown in Table 15.

TABLE 15

| | Formulation # | | | |
|---|---|---|---|---|
| | 171018N | 180225S | 180806S | 180304S |
| | Process | | | |
| | | | | Alternative |
| | | | | holding |
| | | | | process |
| | Continuous | Continuous | | (30 min at |
| Time and | heating- | heating- | | 55-58° |
| Temperature | cooling # | cooling # | Holding | C.) ## |
| T0 5° C. | S | S | S | S |
| T0 25° C. | S | S | S | S |
| T0 30° C. | S | NA | NA | NA |
| T0 40° C. | S | S | S | S |
| 2 Weeks 5° C. | NA | S | S | S |
| 2 Weeks 25° C. | NA | S | S | M |
| 2 Weeks 40° C. | NA | M | M | M |
| 1 Month 5° C. | S | S | S | S |
| 1 Month 25° C. | N | M | S | M |
| 1 Month 30° C. | N | NA | NA | NA |
| 1 Month 40° C. | N | M | M | M |
| 2 Month 5° C. | NA | S | S | S |
| 2 Month 25° C. | N | M | S | M |
| 2 Month 30° C. | N | NA | NA | NA |
| 2 Month 40° C. | N | M | M | N |
| 3 Month 5° C. | M | S | S | S |
| 3 Month 25° C. | N | N | S | N |
| 3 Month 30° C. | N | NA | NA | NA |
| 3 Month 40° C. | N | S | M | NA |
| 5 Month 5° C. | NA | S | NA | NA |
| 5 Month 25° C. | NA | M | NA | NA |
| 5 Month 40° C. | NA | N | NA | NA |
| 6 Month 5° C. | M | S | S | S |
| 6 Month 25° C. | N | M | S | M |
| 6 Month 30° C. | NA | NA | NA | NA |
| 6 Month 40° C. | N | N | NA | NA |

“shakable” (S), “moderately shakable” (M), “Non-shakable” (N) “Non-available” (NA), as defined in the experimental method section.
# For formulations 171018N and 180225S, continuous heating and cooling process was performed as described in Example 1A. Addition of retinoids was performed at about 35-40° C. and there was no overnight mixing step.
## Alternative holding process for 180304S formulation was performed as follows:
Step 1: Some of the hydrophobic solvents and solid compounds such as fatty alcohols, fatty acids and waxes (for example soybean oil, HCO, beeswax) were mixed and heated to a temperature sufficient to achieve complete melting.
Step 2: The formulation was cooled down to about 55-58° C. and was mixed under this temperature for a period of 30 min.
Step 3: The formulation was heated to about 65° C. and the rest of the hydrophobic solvents and solid compounds were added and mixed until completely melting. The process then continued as described in Example 1b step 3 onwards.

As shown in Table 15, a formulation containing a combination of MCH and ADP with 2% HCO prepared by a continuous heating-cooling process (171018N) was largely non-shakable from one month to six months. A formulation containing MCH and ADP with 1.2% HCO prepared by a continuous heating-cooling process (180225S) showed a moderate improvement in shakability compared to a formulation with 2% HCO. A formulation containing MCH and ADP with 1.2% HCO prepared by a holding process (4 hours at about 54° C.) showed a significant improvement in shakability and was shakable at all timepoints tested at 25° C. A formulation containing MCH and ADP with 1.2% HCO prepared by a holding process of 30 minutes at about 55-58° C. and then heating to about 65° C., showed a shakability similar to that measured in a formulation prepared by a continuous heating-cooling process. A non-shakable formulation may over time potentially become non-flowable and might lead e.g., to occurrences of blockage of valves and/or nozzles. These results indicate a reduction in HCO may be helpful in improving shakability but may not completely prevent blockage in a valve or nozzle. The combination of HCO reduction and a holding process (e.g., 4 h at about 54° C.) was successful in producing a shakable formulation at 25° C. which should extrapolate into elimination and/or prevention of potential occurrences of valve blockage. Thus, without being bound by theory, it may be that the presence of the Tmh crystals and a reduction in HCO each contributed to improve shakability and help to prevent valve block.

As can be seen in Table 16, a reduction in HCO amount and holding process did not affect foam quality. All formulations tested had a desirable collapse time and foam quality.

TABLE 16

| | | Formulation # | | |
|---|---|---|---|---|
| | | 171018N | 180225S | 180806S |
| | | Process | | |
| | | Continuous | Continuous | |
| | | heating- | heating- | |
| | | cooling # | cooling# | Holding |
| T0 5° C. | Foam | E | E | E |
| | quality | | | |
| | Collapse | 170 | >180 | 170 |
| | time | | | |
| | Time to | 150 | 180 | 180 |
| | FG | | | |
| T0 25° C. | Foam | E | E | E |
| | quality | | | |
| | Collapse | 170 | >180 | 170 |
| | time | | | |
| | Time to | 150 | 180 | 180 |
| | FG | | | |
| T0 30° C. | Foam | E | NA | NA |
| | quality | | | |
| | Collapse | 170 | NA | NA |
| | time | | | |
| | Time to | 150 | NA | NA |
| | FG | | | |
| T0 40° C. | Foam | E | E | E |
| | quality | | | |
| | Collapse | 170 | >180 | 170 |
| | time | | | |
| | Time to | 150 | 180 | 180 |
| | FG | | | |
| 2 W 5° C. | Foam | NA | E | E |
| | quality | | | |
| | Collapse | NA | >180 | >180 |
| | time | | | |
| | Time to | NA | >180 | 180 |
| | FG | | | |
| 2 W 25° C. | Foam | NA | E | E |
| | quality | | | |
| | Collapse | NA | >180 | >180 |
| | time | | | |
| | Time to | NA | >180 | >180 |
| | FG | | | |
| 2 W 40° C. | Foam | NA | E | E |
| | quality | | | |
| | Collapse | NA | >180 | >180 |
| | time | | | |
| | Time to | NA | >180 | >180 |
| | FG | | | |
| 1 M 5° C. | Foam | E | E | E |
| | quality | | | |
| | Collapse | >180 | >180 | >180 |
| | time | | | |
| | Time to | >180 | >180 | 180 |
| | FG | | | |
| 1 M 25° C. | Foam | E | E | E |
| | quality | | | |
| | Collapse | >180 | >180 | >180 |
| | time | | | |
| | Time to | >180 | >180 | >180 |
| | FG | | | |
| 1 M 30° C. | Foam | E | NA | NA |
| | quality | | | |
| | Collapse | >180 | NA | NA |
| | time | | | |
| | Time to | >180 | NA | NA |
| | FG | | | |
| 1 M 40° C. | Foam | E | E | E |
| | quality | | | |
| | Collapse | >180 | >180 | >180 |
| | time | | | |
| | Time to | >180 | >180 | >180 |
| | FG | | | |
| 2 M 5° C. | Foam | NA | E | E |
| | quality | | | |
| | Collapse | NA | >180 | >180 |
| | time | | | |
| | Time to | NA | >180 | 180 |
| | FG | | | |
| 2 M 25° C. | Foam | E | E | E |
| | quality | | | |
| | Collapse | >180 | >180 | >180 |
| | time | | | |
| | Time to | 150 | >180 | >180 |
| | FG | | | |
| 2 M 30° C. | Foam | E | NA | NA |
| | quality | | | |
| | Collapse | >180 | NA | NA |
| | time | | | |
| | Time to | >180 | NA | NA |
| | FG | | | |
| 2 M 40° C. | Foam | E | E | E |
| | quality | | | |
| | Collapse | >180 | >180 | >180 |
| | time | | | |
| | Time to | 150 | >180 | 180 |
| | FG | | | |
| 3 M 5° C. | Foam | E | E | E |
| | quality | | | |
| | Collapse | >180 | >180 | >180 |
| | time | | | |
| | Time to | >180 | >180 | >180 |
| | FG | | | |
| 3 M 25° C. | Foam | E | E | E |
| | quality | | | |
| | Collapse | >180 | >180 | >180 |
| | time | | | |
| | Time to | >180 | >180 | >180 |
| | FG | | | |
| 3 M 30° C. | Foam | E | NA | NA |
| | quality | | | |
| | Collapse | >180 | NA | NA |
| | time | | | |
| | Time to | >180 | NA | NA |
| | FG | | | |
| 3 M 40° C. | Foam | E | E | E |
| | quality | | | |
| | Collapse | >180 | >180 | >180 |
| | time | | | |
| | Time to | >180 | >180 | >180 |
| | FG | | | |
| 4 M 5° C. | Foam | NA | NA | NA |
| | quality | | | |
| | Collapse | NA | NA | NA |
| | time | | | |
| | Time to | NA | NA | NA |
| | FG | | | |
| 4 M 25° C. | Foam | NA | NA | NA |
| | quality | | | |
| | Collapse | NA | NA | NA |
| | time | | | |
| | Time to | NA | NA | NA |
| | FG | | | |
| 4 M 40° C. | Foam | E | NA | NA |
| | quality | | | |
| | Collapse | >180 | NA | NA |
| | time | | | |
| | Time to | >180 | NA | NA |
| | FG | | | |
| 5 M 5° C. | Foam | NA | NA | NA |
| | quality | | | |
| | Collapse | NA | >180 | NA |
| | time | | | |
| | Time to | NA | >180 | NA |
| | FG | | | |
| 5 M 25° C. | Foam | NA | NA | NA |
| | quality | | | |
| | Collapse | NA | >180 | NA |
| | time | | | |
| | Time to | NA | >180 | NA |
| | FG | | | |
| 5 M 40° C. | Foam | NA | NA | NA |
| | quality | | | |
| | Collapse | NA | >180 | NA |
| | time | | | |
| | Time to | NA | >180 | NA |
| | FG | | | |
| 6 M 5° C. | Foam | E | E | E |
| | quality | | | |
| | Collapse | >180 | >180 | 150 |
| | time | | | |
| | Time to | >180 | >180 | 150 |
| | FG | | | |
| 6 M 25° C. | Foam | E | E | E |
| | quality | | | |
| | Collapse | >180 | >180 | 120 |
| | time | | | |
| | Time to | >180 | 180 | 90 |
| | FG | | | |
| 6 M 40° C. | Foam | E | E | NA |
| | quality | | | |
| | Collapse | >180 | >180 | NA |
| | time | | | |
| | Time to | >180 | >180 | NA |
| | FG | | | |

### Example 7

Formulations Prepared by Different Processes and their Shakability

This example presents an alternative continuation process that did not result in crystals with the crystallization fingerprint seen with the holding process discussed above and presents alternative holding processes which produced foamable formulations with less good shakability compared to a formulation prepared by a holding process.

Formulations comprising MCH+ADP with either 1.2% or 2% HCO were prepared by different processes. As shown in Table 17, an alternative holding process (a), comprising holding at about 52° C. for 4 hours and then heating back to about 65° C. (190512N) resulted in improved shakability as compared to a continuous heating-cooling process, but was less effective than holding at about 54° C. for 4 hours (Table 17, compared to Table 11A in Example 3). For example, on Day 15 at 25° C., the alternative holding process resulted in only a moderately-shakable formulation whereas holding at about 54° C. resulted in a fully shakable formulation.

A continuous process (b) wherein the HCO is added at 22° C., followed by a continuous heating-cooling procedure (190512S) resulted in moderately-shakable or non-shakable formulations.

A formulation prepared by another alternative holding process (c), i.e. holding for 30 min at 55-58° C. was moderately shakable on Day 30 at 25° C.

Such formulations prepared by the continuous process (b) could create, with time, a stiff and hard gel eventually leading to blockage of an aerosol valve.

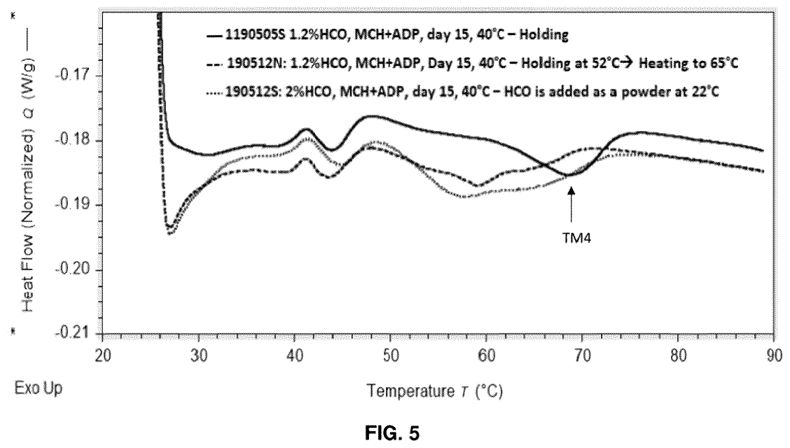

Formulations prepared by an alternative holding process (holding at about 52° C. and heating to about 65° C.; 190512N) or a process where HCO is added at about 22° C. (190512S) showed DSC thermograms where T_{M4} faded and new transitions appeared. Such DSC thermograms were different from those measured for formulations prepared by a holding process at about 54° C. (FIG. 5). It may be, without being bound by theory, that warming to a higher temperature after a holding period may reduce or eliminate the Tmh crystals,

Alternative holding processes were also not as effective in forming (or retaining when reheated) stable structures and crystal fingerprint comprising Tmh crystals, and thus, they did not result in a fully shakable formulation. Shakable formulations may present multiple advantages for a foamable formulation, including awareness of the formulation in the canister, ability to ensure the contents remains homogenous, awareness when the canister is nearly empty and avoidance of a potential for the contents to solidify in the canister or to form a blockage.

TABLE 17

| | | | |
|---|---|---|---|
| Study | 3.12 | 3.13 | |
| Formulation # | 190512N | 190512S | 180304S |
| HCO amount | 1.2% | 2% | 1.2% |
| API | MCH + ADP | MCH + ADP | MCH + ADP |
| Process | Alternative holding | Continuous | Alternative holding |
| | process^{a} | heating-cooling | process^{c} (30 min at |
| | (4 h at 52° C.) | process^{b}; (HCO | 55-58° C.) |
| | | added at 22° C.) | |
| Component | | | |
| Soybean oil | 50 | 50 | 50 |
| Coconut oil | 23.6 | 23.6 | 23.6 |
| Light mineral oil* | 3.3 | 2.5 | 2.5 |
| Stearic acid | 3 | 3 | 3 |
| Docosanol | 1.1 | 1.1 | 1.1 |
| Hydrogenated castor oil | 1.2 | 2 | 1.2 |
| White wax (beeswax) | 2 | 2 | 2 |
| Stearyl alcohol | 1.5 | 1.5 | 1.5 |
| Cetostearyl alcohol | 3.5 | 3.5 | 3.5 |
| Myristyl alcohol | 2.5 | 2.5 | 2.5 |
| Cyclomethicone | 5 | 5 | 5 |
| Minocycline HCl** | 3 | 3 | 3 |
| Adapalene | 0.3 | 0.3 | 0.3 |
| TOTAL | 100 | 100 | 100 |

| Test | Tm/Time | | T0 | T15 | T30 | T0 | T15 | T30 | T0 | T15 | T30 |
|---|---|---|---|---|---|---|---|---|---|---|---|
| Shakability | 5° | C. | S | S | S | S | M | S | S | S | S |
| | 25° | C. | S | M | S | S | M | | S | M | M |
| | 40° | C. | S | M | M | S | N | N | S | M | M |

“shakable” (S), “moderately shakable” (M), “Non-shakable” (N) “Non-available” (NA), as defined in the experimental method section.
^{a}Alternative holding process for 190512N formulation was performed as follows:
Step 1: Some of the hydrophobic solvents and solid compounds such as fatty alcohols, fatty acids and waxes (for e.g. soybean oil, HCO, beeswax and Docosanol) were mixed and heated to a temperature sufficient to achieve complete melting.
Step 2: The formulation was cooled down to 52° C. and was mixed under this temperature for a period of 4 hours.
Step 3: The formulation was heated to 65° C. and the rest of the hydrophobic solvents and solid compounds were added and mixed until complete melting. The process then continued as described in Example 1b step 3 onwards.
^{b}Continuous heating cooling process for 190512S formulation was performed as follows: Continuous heating and cooling process was performed as described in Example 1a. Hydrogenated castor oil was added as a powder at about 22° C.
^{c}Alternative holding process for 180304S formulation was performed as follows:
Step 1: Some of the hydrophobic solvents and solid compounds such as fatty alcohols, fatty acids and waxes (for e.g. soybean oil, HCO, beeswax) were mixed and heated to a temperature sufficient to achieve complete melting.
Step 2: The formulation was cooled down to about 55-58° C. and was mixed under this temperature for a period of 30 min.
Step 3: The formulation was heated to about 65° C. and the rest of the hydrophobic solvents and solid compounds was added and mixed until complete melting. The process then continued as described in Example 1b Step 3 onwards.

### Example 8

### Formulations Manufactured by a Holding Process at Different Temperatures

This example evaluates formulations prepared by a holding process, where the holding was performed at different temperatures.

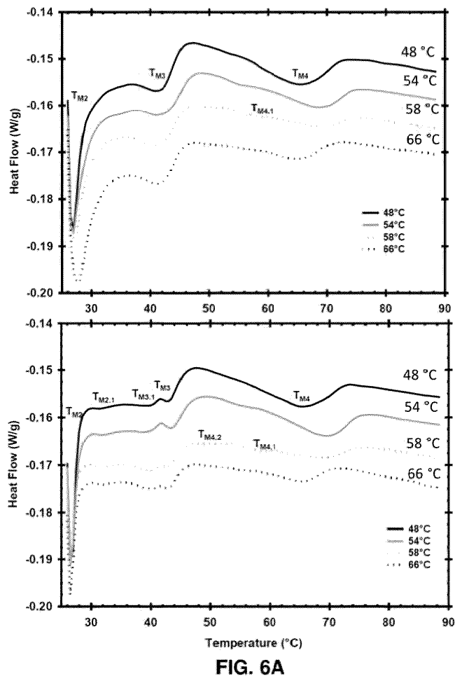

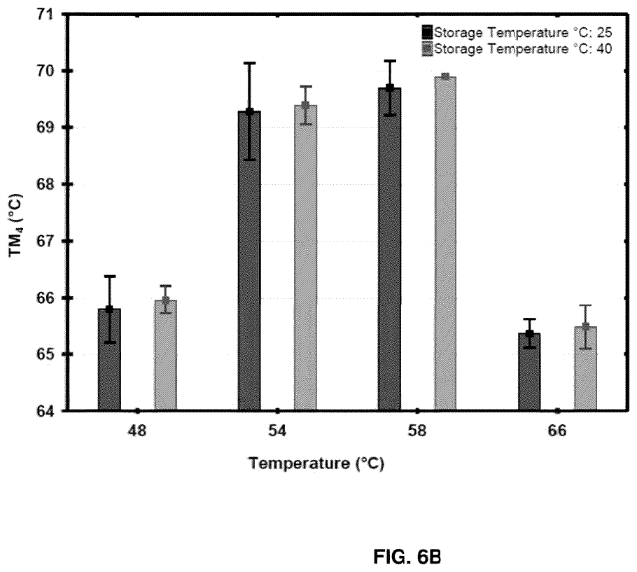

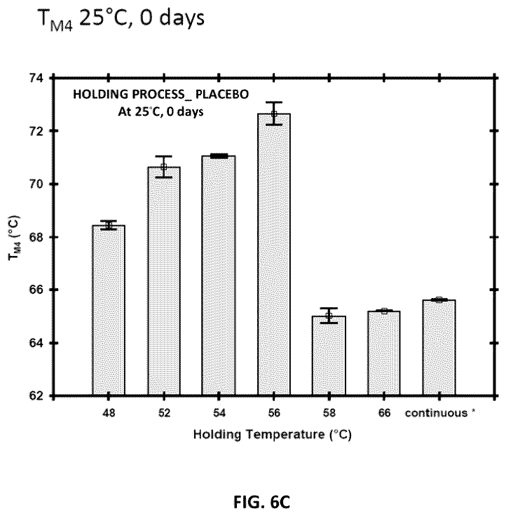

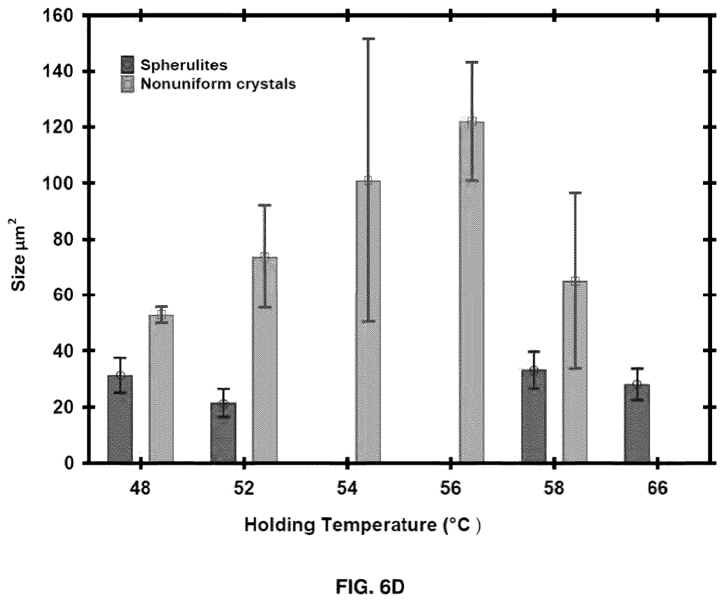

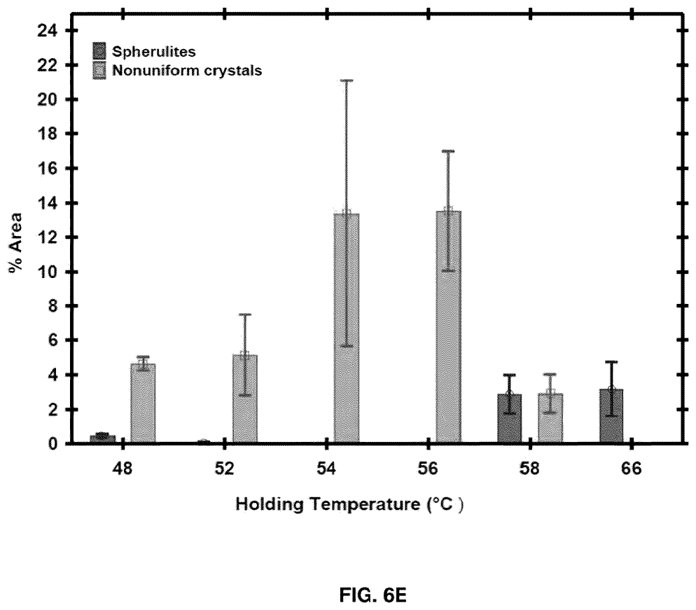

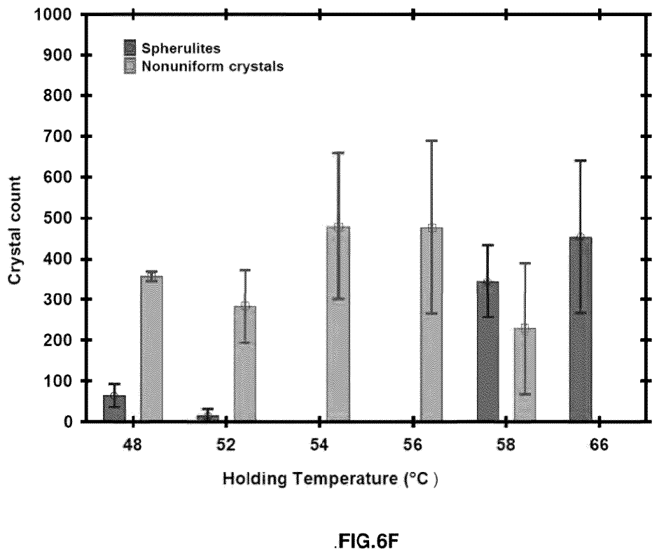

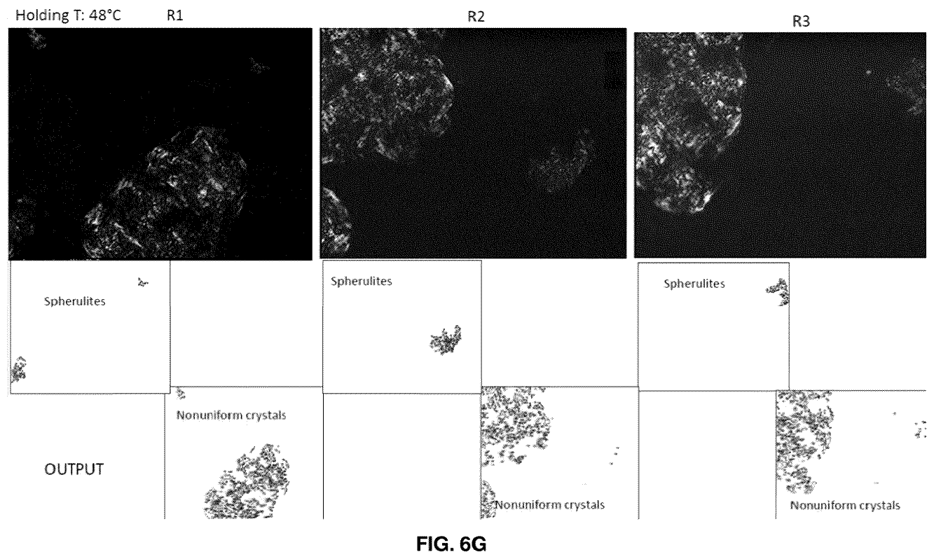

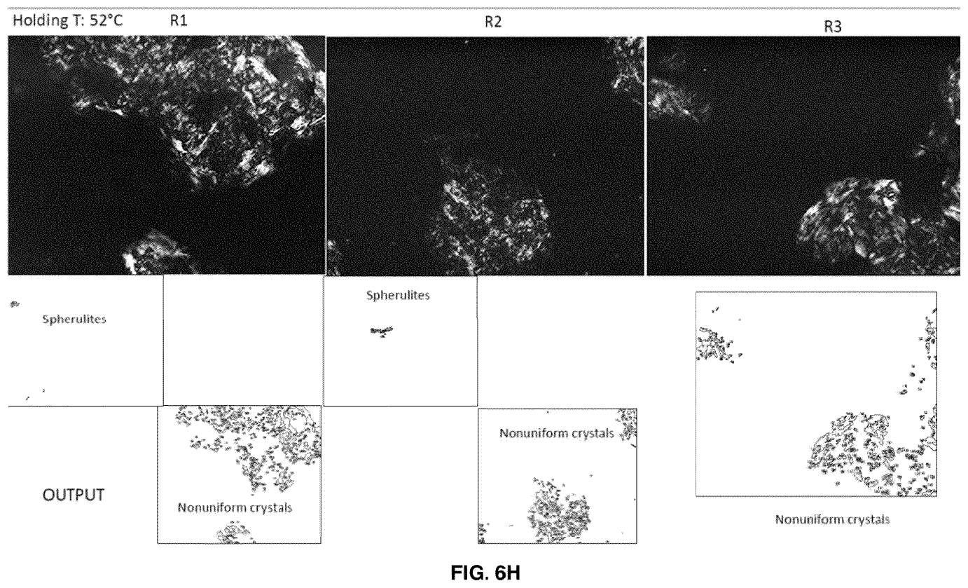

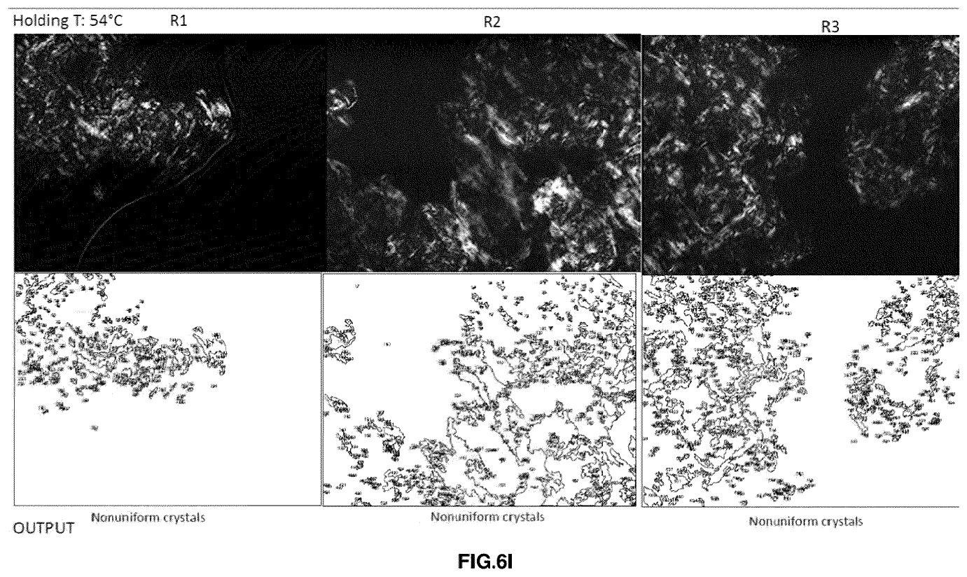

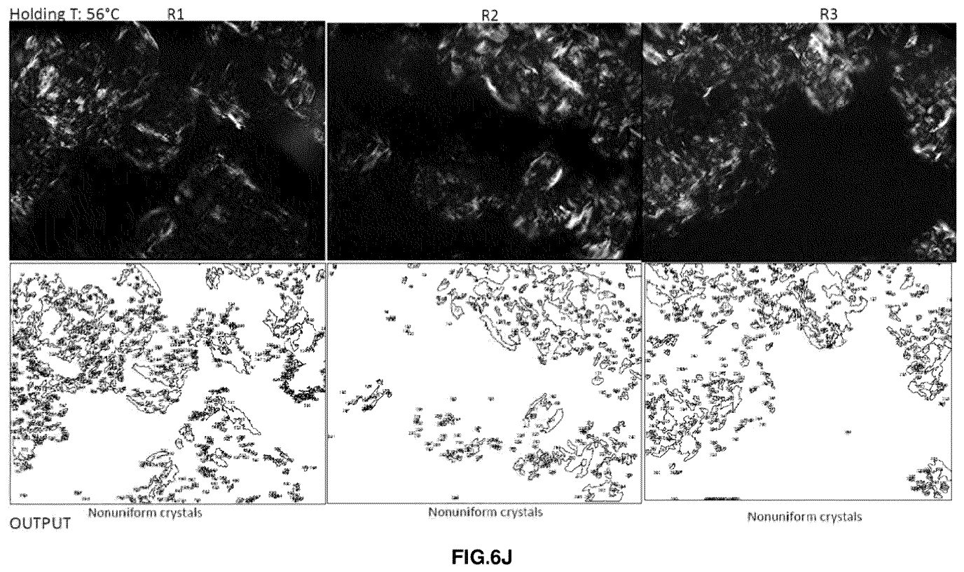

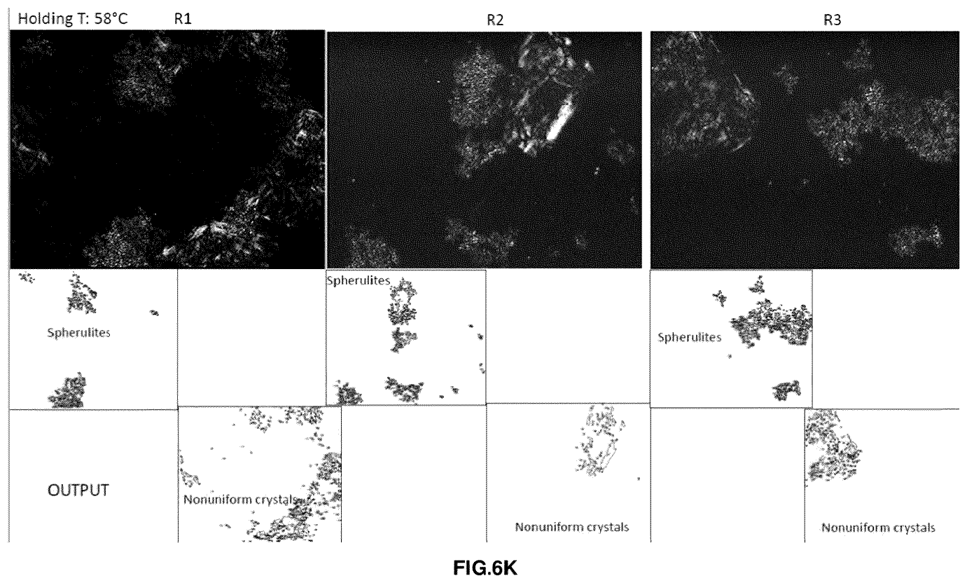

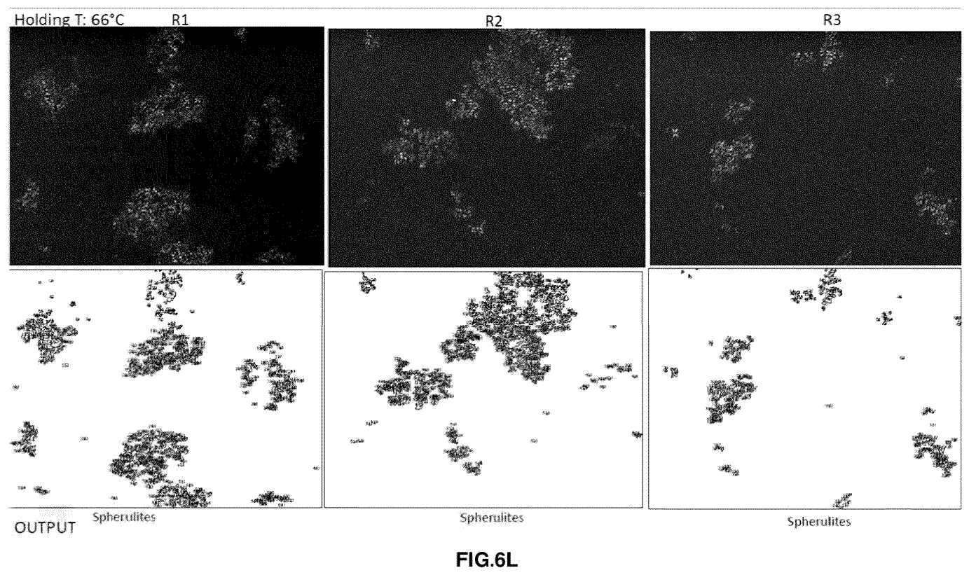

Formulations containing 1.2% hydrogenated castor oil (HCO) and active agents comprising a combination of minocycline HCL and adapalene (MCH+ADP) were prepared by a holding process, where the holding step was performed at different temperatures as described in Table 18. As can be seen in Table 18 and FIG. 6A, the holding temperature had a significant impact on the DSC pattern of the tested formulations. A major difference was observed at the T_{M4} transition. For formulations tested on Day 0, kept at 25° C. and 40° C., holding temperatures of about 54° C. (190505S) and about 58° C. (190618R) successfully increased T_{M4}, (about 69-70° C.), resulting in crystals of higher structural order. On the contrary, holding temperatures of about 48° C. (190616S) and about 66° C. (190623N) did not raise T_{M4}, (about 65-66° C.) resulting in T_{M4} temperatures similar to those found in a continuous heating-cooling process (see Example 3).

Without being bound by theory, at lower holding temperatures (e.g., about 48° C.), the crystallization process may have started prior to the holding. As a result, crystal nuclei may already have formed allowing further crystallization seeded by those nuclei to proceed (whereas at a higher holding temperature a different crystallization process may take place). At higher holding temperatures (e.g., about 66° C.) which are closer to the melting temperature of HCO in such formulations, the thermodynamic drive for a structural order of the molecules may be too low or the holding time of 4 hours at this temperature may be too short. FIG. 6B shows a clear difference between T_{M4} in formulations prepared at holding temperatures of about 54° C. and about 58° C. compared to those prepared at holding temperatures of about 48° C. and about 66° C.

Formulations containing 1.2% hydrogenated castor oil (HCO) without active agents were prepared by a holding process, where the holding step was performed at different temperatures as described in Table 18. As can be seen in FIG. 6C and tables 18D and 18E, for placebo formulations, the holding temperature of about 56° C. seemed to be the optimal temperature where T_{M4} resulted the highest. In addition, as can be seen in table 18D, holding temperature of about 54° C. and 56° C. resulted in plates and Tmh crystals, whereas holding temperatures of below 54° C. and above 56° C. resulted in the presence of spherulites that were associated with crystals of lower structural order. Thus, in one or more embodiments an optimal holding temperature is between about 54-56° C.

Photomicrographs taken at 50° C. from placebo samples at day 0 heated from 25 to 50° C. were analyzed through Image J to obtain the spherulites/Tmh crystals ratio. As can be seen in Tables 18E and 18F, Tmh crystals were present in formulations prepared by holding temperatures of about 48° C. to about 58° C., but not in a formulation prepared by a holding temperature of 66° C. The relative percentage of area held by these Tmh crystals in formulations prepared by holding temperatures of about 48° C. to about 58° C. was above 70% compared to the relative percentage area held by the spherulites which was below 30%. Thus, in one or more embodiments, the holding temperature is between about 48° C. to about 58° C.

Additional replicates from samples stored at 5° C. were conducted. Images were analyzed as was done previously in Image J software at a minimum of 3 μm and for each image the zones of the different structural units were visually selected and then measured for crystal count, size and % of Area. Three independent microphotographs were used for each preparation condition. As can be seen in Tables 18 D-G, and FIGS. 6G to 6L, Tmh crystals are bigger than spherulites independent of the holding temperature. In addition, Tmh crystals are higher in number and cover a higher % of the area of the photomicrograph, except for the sample with a 58° C. holding temperature, where spherulites and Tmh crystals are present approximately in the same amount and % Area as Tmh crystals. The only sample where Tmh crystals are not present is the sample manufactured under 66° C. holding, while the samples with holding temperature of 54° C. and 56° C. exhibit the highest values for Tmh crystals (as measured by crystal count, size, and % Area). Similar improvements are observed in each property in both placebo formulations and formulations with active ingredient.

TABLE 18A

| Study | | | 4.1 | | | 4.2 | | | 3.9 | | | 3.11 | | |
|---|---|---|---|---|---|---|---|---|---|---|---|---|---|---|
| Formulation # | | | 190616S | | | PH48 | | | 190505S | | | PH54 | | |
| HCO amount | | | 1.2% | | | 1.2% | | | 1.2% | | | 1.2% | | |
| API | | | MCH + ADP | | | Placebo | | | MCH + ADP | | | Placebo | | |
| Process | | | Holding at 48° C. | | | Holding at 48° C. | | | Holding at 54° C. | | | Holding at 54° C. | | |
| Component | | | | | | | | | | | | | | |
| Soybean oil | | | 50 | | | 50 | | | 50 | | | 50 | | |
| coconut oil | | | 23.6 | | | 23.6 | | | 23.6 | | | 23.6 | | |
| Light mineral oil* | | | 3.3 | | | 6.6 | | | 3.3 | | | 6.6 | | |
| Stearic acid | | | 3 | | | 3 | | | 3 | | | 3 | | |
| Docosanol | | | 1.1 | | | 1.1 | | | 1.1 | | | 1.1 | | |
| Hydrogenated castor oil | | | 1.2 | | | 1.2 | | | 1.2 | | | 1.2 | | |
| White wax (beeswax) | | | 2 | | | 2 | | | 2 | | | 2 | | |
| Stearyl alcohol | | | 1.5 | | | 1.5 | | | 1.5 | | | 1.5 | | |
| Cetostearyl alcohol | | | 3.5 | | | 3.5 | | | 3.5 | | | 3.5 | | |
| Myristyl alcohol | | | 2.5 | | | 2.5 | | | 2.5 | | | 2.5 | | |
| Cyclomethicone | | | 5 | | | 50 | | | 5 | | | 5 | | |
| Minocycline HCl** | | | 3 | | | 0 | | | 3 | | | 0 | | |
| Adapalene | | | 0.3 | | | 0 | | | 0.3 | | | 0 | | |
| TOTAL | | | 100 | | | 100 | | | 100 | | | 100 | | |
| | Temperature/ | | | | | | | | | | | | | |
| Test | Time | | T0 | T15 | T30 | T0 | T15 | T30 | T0 | T15 | T30 | T0 | T15 | 130 |
| Shakability | 5° | C. | | S | S | NA | NA | NA | S | S | S | NA | NA | NA |
| | 25° | C. | | S | S | NA | NA | NA | S | S | S | NA | NA | NA |
| | 40° | C. | | M | M | NA | NA | NA | S | M | M | NA | NA | NA |
| DSC | 5° | C. | NA | 27.0 | 27.1 | NA | 27.5 | 27.4 | NA | 27.1 | 26.7 | NA | 26.7 | 127.0 |
| TM2 | | | | 0.0 | 0.0 | | 0.7 | 0.3 | | 0.1 | 0.1 | | 0.1 | 0.3 |
| | 25° | C. | 27.0 | 27.2 | 27.3 | 27.5 | 28.3 | | 26.9 | 27.1 | 27.0 | 26.7 | 26.7 | 26.5 |
| | | | 0.3 | 0.1 | 0.1 | 0.1 | 0.1 | | 0.1 | 0.1 | 0.1 | 0.2 | 0.1 | 0.0 |
| | 40° | C. | 26.7 | 27.1 | | 27.1 | 28.3 | | 26.5 | | | 26.9 | 26.5 | |
| | | | 0.2 | 0.0 | | 0.2 | 0.3 | | 0.0 | | | 0.0 | 0.1 | |
| DSC | 5° | C. | NA | | | | | | NA | | | NA | | |
| TM2.1 | 25° | C. | | | | | | 30.7 | | | | | | |
| | | | | | | | | 0.1 | | | | | | |
| | 40° | C. | 31.5 | | 30.4 | | | | 31.9 | 29.2 | 30.3 | 33.0 | 34.0 | 34.5 |
| | | | 0.2 | | 0.0 | | | | 0.1 | 2.0 | 0.3 | 0.2 | 0.3 | 0.2 |
| DSC | 5° | C. | NA | | | | | | NA | | | NA | 35.2 | 35.0 |
| TM3.1 | | | | | | | | | | | | | 0.3 | 0.1 |
| | 25° | C. | | | | | | | | | | 35.4 | 36.8 | |
| | | | | | | | | | | | | 0.0 | 0.1 | |
| | 40° | C. | 39.6 | | 38.3 | | | | 39.9 | 38.3 | 38.8 | | | |
| | | | 0.1 | | 0.0 | | | | 0.3 | 0.1 | 0.3 | | | |
| DSC | 5° | C. | NA | 42.2 | 41.7 | NA | 44.0 | 43.3 | NA | 42.4 | 42.1 | NA | 42.4 | 43.0 |
| TM3 | | | | 0.1 | 0.0 | | 0.01 | 0.3 | | 0.1 | 0.1 | | 0.2 | 0.1 |
| | 25° | C. | 41.6 | 41.8 | 42.1 | 43.3 | 42.5 | 43.0 | 42.1 | 42.3 | 41.9 | 42.8 | 43.1 | 41.2 |
| | | | 0.3 | 0.0 | 0.2 | 0.3 | 0.6 | 0.1 | 0.2 | 0.7 | 0.2 | 0.2 | 0.1 | 1.2 |
| | 40° | C. | 43.0 | 43.2 | 43.4 | 44.4 | 44.3 | 43.2 | 43.5 | 43.8 | 44.1 | 43.4 | 43.1 | 43.7 |
| | | | 0.1 | 0.0 | 0.1 | 0.1 | 0.3 | 0.2 | 0.1 | 0.1 | 0.2 | 0.1 | 0.2 | 0.0 |
| DSC | 5° | C. | NA | | | | | | NA | | | NA | 51.2 | |
| TM4.2 | | | | | | | | | | | | | 0.1 | |
| | 25° | C. | | | | | | | | | | 50.7 | | |
| | | | | | | | | | | | | 0.5 | | |
| | 40° | C. | | | | | | | | 53.7 | 56.1 | 51.1 | | |
| | | | | | | | | | | 0.1 | 0.5 | 0.0 | | |
| DSC | 5° | C. | NA | | | | | | NA | | | NA | | 58.7 |
| TM4.1 | | | | | | | | | | | | | | 0.3 |
| | 25° | C. | | | | | | | | | | 58.4 | 59.0 | 58.9 |
| | | | | | | | | | | | | 0.3 | 0.5 | 0.4 |
| | 40° | C. | | | | | 61.4** | | | | | 58.5 | 58.7 | 58.6 |
| | | | | | | | | | | | | 0.0 | 0.8 | 0.1 |
| DSC | 5° | C. | NA | 64.7 | 65.2 | NA | 68.6 | 68.6 | NA | 68.5 | 68.6 | NA | 71.5 | 70.6 |
| TM4 | | | | 0.0 | 0.5 | | 0.3 | 0.2 | | 0.4 | 0.2 | | 0.2 | 0.3 |
| | 25° | C. | 65.8 | 65.5 | 65.7 | 68.5 | 68.5 | 68.2 | 69.3 | 69.2 | 68.8 | 71.1 | 71.0 | 70.0 |
| | | | 0.6 | 0.2 | 0.3 | 0.2 | 0.2 | 0.3 | 0.8 | 0.2 | 0.5 | 0.1 | 0.3 | 1.0 |
| | 40° | C. | 66.0 | 64.7 | 64.5 | 68.6 | 69.4 | 68.0 | 69.4 | 68.4 | 68.6 | 71.5 | 70.8 | 70.6 |
| | | | 0.2 | 0.0 | 0.3 | 0.2 | 0.2 | 0.0 | 0.3 | 0.2 | 0.4 | 0.1 | 0.0 | 0.0 |

**Parameter present in only one replicate

TABLE 18B

| Study | | | 4.9 | | | 4.10 | | | 4.11 | | | 4.12 | | |
|---|---|---|---|---|---|---|---|---|---|---|---|---|---|---|
| Formulation # | | | 190618R | | | PH58 | | | 190623N | | | PH66 | | |
| HCO amount | | | 1.2% | | | 1.2% | | | 1.2% | | | 1.2% | | |
| API | | | MCH + ADP | | | Placebo | | | MCH + ADP | | | Placebo | | |
| Process | | | Holding at 58° C. | | | Holding at 58° C. | | | Holding at 66° C. | | | Holding at 66° C. | | |
| Component | | | | | | | | | | | | | | |
| Soybean oil | | | 50 | | | 50 | | | 50 | | | 50 | | |
| coconut oil | | | 23.6 | | | 23.6 | | | 23.6 | | | 23.6 | | |
| Light mineral oil** | | | 3.3 | | | 6.6 | | | 3.3 | | | 6.6 | | |
| Stearic acid | | | 3 | | | 3 | | | 3 | | | 3 | | |
| Docosanol | | | 1.1 | | | 1.1 | | | 1.1 | | | 1.1 | | |
| Hydrogenated castor oil | | | 1.2 | | | 1.2 | | | 1.2 | | | 1.2 | | |
| White wax (beeswax) | | | 2 | | | 2 | | | 2 | | | 2 | | |
| Stearyl alcohol | | | 1.5 | | | 1.5 | | | 1.5 | | | 1.5 | | |
| Cetostearyl alcohol | | | 3.5 | | | 3.5 | | | 3.5 | | | 3.5 | | |
| Myristyl alcohol | | | 2.5 | | | 2.5 | | | 2.5 | | | 2.5 | | |
| Cyclomethicone | | | 5 | | | 5 | | | 5 | | | 5 | | |
| Minocycline HCl*** | | | 3 | | | 0 | | | 3 | | | 0 | | |
| Adapalene | | | 0.3 | | | 0 | | | 0.3 | | | 0 | | |
| TOTAL | | | 100 | | | 100 | | | 100 | | | 100 | | |
| | Temperature/ | | | | | | | | | | | | | |
| Test | Time | | T0 | T15 | T30 | T0 | T15 | T30 | T0 | T15 | T30 | T0 | T15 | T30 |
| Shakability | 5° | C. | | S | S | NA | NA | NA | | S | S | NA | NA | NA |
| | 25° | C. | | S | S | NA | NA | NA | | S | S | NA | NA | NA |
| | 40° | C. | | N | M | NA | NA | NA | | N | N | NA | NA | NA |
| DSC TM2 | 5° | C. | NA | 27.5 | 27.3 | NA | 27.1 | 26.9 | NA | 27.4 | 26.9 | NA | 27.5 | 27.4 |
| | | | | 0.2 | 0.1 | | 0.3 | 0.0 | | 0.1 | 0.2 | | 0.3 | 0.5 |
| | 25° | C. | 27.3 | 27.0 | 28.3 | 27.2 | 26.7 | 26.5 | 27.8 | 27.3 | 28.1 | 27.5 | 27.4 | 28.4 |
| | | | 0.2 | 0.1 | 0.2 | 0.2 | 0.6 | 0.0 | 0.2 | 0.3 | 0.2 | 0.1 | 0.0 | 0.7 |
| | 40° | C. | 26.4* | 27.1 | | 27.3 | 126.6 | | 26.7 | 27.6 | | 27.3 | 26.5 | |
| | | | | 0.2 | | 0.2 | 0.0 | | 0.1 | 0.3 | | 0.2 | 0.3 | |
| DSC TM2.1 | 5° | C. | NA | | | | | | NA | | | NA | 30.6 | |
| | | | | | | | | | | | | | 0.4 | |
| | 25° | C. | | | | | | | | | | 29.8 | | |
| | | | | | | | | | | | | 0.7 | | |
| | 40° | C. | 31.6* | | | | | 33.4 | 31.5 | | | 32.2 | 33.0 | |
| | | | | | | | | 1.3 | 0.1 | | | 1.6 | 0.3 | |
| DSC TM3.1 | 5° | C. | NA | | | | | | NA | | | | | |
| | 25° | C. | | | 35.7 | | 37.1 | 36.7 | | 35.4 | 36.1 | | | |
| | | | | | 0.6 | | 0.7 | 0.1 | | 0.4 | 0.1 | | | |
| | 40° | C. | 40.0* | 38.6 | 38.2 | | | | 40.0 | | 36.7 | | | |
| | | | | 0.2 | 0.2 | | | | 0.1 | | 0.1 | | | |
| DSC TM3 | 5° | C. | NA | 41.9 | 41.7 | NA | 43.1 | 43.0 | NA | 42.2 | 42.1 | NA | 43.5 | 43.7 |
| | | | | 0.1 | 0.2 | | 0.2 | 0.1 | | 0.1 | 0.0 | | 0.1 | 0.4 |
| | 25° | C. | 41.4 | 42.2 | 41.7 | 43.0 | 42.8 | 43.1 | 41.4 | 42.3 | 42.6 | 43.4 | 43.3 | 44.2 |
| | | | 0.5 | 0.3 | 0.1 | 0.2 | 0.1 | 0.5 | 0.1 | 0.1 | 0.0 | 0.3 | 0.2 | 0.0 |
| | 40° | C. | 42.9* | 43.6 | 43.8 | 43.5 | 42.8 | 43.4 | 43.0 | 43.3 | 43.5 | 44.4 | 43.3 | 43.9 |
| | | | | 0.0 | 0.1 | 0.0 | 0.1 | 0.6 | 0.1 | 0.1 | 0.0 | 0.0 | 0.1 | 2.5 |
| DSC TM4.2 | 5° | C. | NA | | | | | | NA | | | | | |
| | 25° | C. | | | | | | | | | | | | |
| | 40° | C. | 49.2* | | | | | | | | | | | |
| DSC TM4.1 | 5° | C. | NA | 58.1 | 58.3 | | | | NA | | | | | |
| | | | | 0.2 | 0.1 | | | | | | | | | |
| | 25° | C. | 58.3 | 58.6 | 59.2 | | | | | | | | | |
| | | | 0.1 | 0.5 | 0.3 | | | | | | | | | |
| | 40° | C. | 58.8* | 56.8 | 55.5 | | 59.0 | | | 58.2 | 60.0 | | | |
| | | | | 4.3 | 1.2 | | 0.0 | | | 0.5 | 0.6 | | | |
| DSC TM4 | 5° | C. | NA | 68.7 | 68.0 | NA | 65.2 | 64.6 | NA | 65.8 | 65.7 | NA | 65.1 | 65.0 |
| | | | | 0.3 | 0.5 | | 0.1 | 0.0 | | 0.2 | 0.1 | | 0.4 | 0.1 |
| | 25° | C. | 69.7 | 69.5 | 67.9 | 65.05 | 64.3 | 65.4 | 65.3 | 65.7 | 66.0 | 65.2 | 65.8 | 65.8 |
| | | | 0.5 | 0.1 | 0.4 | 0.3 | 0.3 | 0.4 | 0.2 | 0.1 | 0.2 | 0.0 | 0.9 | 0.2 |
| | 40° | C. | 69.9* | 69.6 | 70.3 | 64.8 | 64.40.1 | 63.3 | 65.5 | 64.4 | 65.40.3 | 65.4 | 65.2 | 66.0 |
| | | | | 0.1 | 0.6 | 0.0 | | 0.7 | 0.4 | 0.1 | | 0.3 | 0.2 | 0.6 |

*Only one replicate was performed.
**The amount of light mineral oil in the formulation is adjusted based on the amount of minocycline hydrochloride and/or adapalene.
***The amount of minocycline hydrochloride in the formulation is adjusted by the potency of the minocycline hydrochloride.

TABLE 18C

| Study | | | | | | | | |
|---|---|---|---|---|---|---|---|---|
| Formulation # | | | PH52 | | | PH56 | | |
| HCO amount | | | 1.2% | | | 1.2% | | |
| API | | | Placebo | | | Placebo | | |
| Process | | | Holding at 52° C. | | | Holding at 56° C. | | |
| Component | | | | | | | | |
| Soybean oil | | | 50 | | | 50 | | |
| coconut oil | | | 23.6 | | | 23.6 | | |
| Light mineral oil** | | | 6.6 | | | 6.6 | | |
| Stearic acid | | | 3 | | | 3 | | |
| Docosanol | | | 1.1 | | | 1.1 | | |
| Hydrogenated castor oil | | | 1.2 | | | 1.2 | | |
| White wax (beeswax) | | | 2 | | | 2 | | |
| Stearyl alcohol | | | 1.5 | | | 1.5 | | |
| Cetostearyl alcohol | | | 3.5 | | | 3.5 | | |
| Myristyl alcohol | | | 2.5 | | | 2.5 | | |
| Cyclomethicone | | | 5 | | | 5 | | |
| Minocycline HCl*** | | | 0 | | | 0 | | |
| Adapalene | | | 0 | | | 0 | | |
| TOTAL | | | 100 | | | 100 | | |
| | Temperature/ | | | | | | | |
| Test | Time | | T0 | T15 | T30 | T0 | T15 | T30 |
| Shakability | 5° | C. | | | | | | |
| | 25° | C. | | | | | | |
| | 40° | C. | | | | | | |
| DSC TM2 | 5° | C. | NA | 27.2 | 27.5 | NA | 27.6 | 27.2 |
| | | | | 0.3 | 0.0 | | 0.2 | 0.0 |
| | 25° | C. | 27.7 | 28.0 | 28.2 | 26.9 | 27.6 | 28.7 |
| | | | 0.4 | 0.3 | 0.7 | 0.1 | 0.0 | 0.0 |
| | 40° | C. | 27.3 | 26.7 | | 26.6 | 26.6 | |
| | | | 0.1 | 0.2 | | 0.1 | 0.3 | |
| DSC TM2.1 | 5° | C. | NA | | | NA | | |
| | 25° | C. | | | | 29.0 | 29.2 | |
| | | | | | | 0.0 | 0.0 | |
| | 40° | C. | | 33.8 | 31.7 | 32.6 | 33.3 | |
| | | | | 0.0 | 0.3 | 0.3 | 0.4 | |
| DSC TM3.1 | 5° | C. | NA | 35.6 | 35.5 | NA | | |
| | | | | 0.0 | 0.1 | | | |
| | 25° | C. | 34.8 | | 38.8 | | | |
| | | | 0.4 | | 0.3 | | | |
| | 40° | C. | 38.7 | | | 39.5 | | |
| | | | 0.3 | | | 0.2 | | |
| DSC TM3 | 5° | C. | NA | 43.3 | 43.4 | NA | 40.1 | 40.0 |
| | | | | 0.1 | 0.0 | | 0.0 | 1.1 |
| | 25° | C. | 43.8 | 43.8 | 43.1 | 140.4 | 40.2 | 40.8 |
| | | | 0.0 | 0.3 | 0.0 | 0.1 | 0.0 | 0.1 |
| | 40° | C. | 44.7 | 44.0 | 44.2 | 43.0 | 41.5 | 40.4 |
| | | | 0.1 | 0.1 | 0.1 | 0.4 | 0.1 | 0.1 |
| DSC TM4.2 | 5° | C. | NA | | | NA | 51.4 | 51.3 |
| | | | | | | | 0.5 | 0.5 |
| | 25° | C. | 55.5 | 55.3 | | 51.1 | 51.3 | |
| | | | 0.1 | 0.1 | | 0.0 | 0.1 | |
| | 40° | C. | 55.7 | 56.8 | | 51.4 | 58.5 | 57.8 |
| | | | 0.2 | 0.1 | | 0.2 | 0.1 | 0.4 |
| DSC TM4.1 | 5° | C. | NA | | | NA | | |
| | 25° | C. | | | | | | |
| | 40° | C. | | | | 58.8 | 62.1 | 64.1 |
| | | | | | | 0.2 | 0.0 | 0.6 |
| DSC TM4 | 5° | C. | NA | 70.4 | 71.4 | NA | 72.9 | 72.7 |
| | | | | 0.2 | 0.1 | | 0.1 | 0.1 |
| | 25° | C. | 70.7 | 71.1 | 71.4 | 72.7 | 72.9 | 72.7 |
| | | | 0.4 | 0.0 | 0.2 | 0.4 | 0.1 | 0.0 |
| | 40° | C. | 70.7 | 70.9 | 70.6 | 73.1 | 71.9 | 72.4 |
| | | | 0.3 | 0.1 | 0.1 | 0.1 | 0.5 | 0.6 |

TABLE 18D

| Holding | |
|---|---|
| T (° C.) | Microstructural units observed 0 Days 25° C. |
| 48 | Plates, spherulites and Tmh crystals |
| 52 | Plates, Tmh crystals and few spherulites |
| 54 | Plates and Tmh crystals |
| 56 | Plates and Tmh crystals |
| 58 | Plates, spherulites and few Tmh crystals |
| 66 | Plates and spherulites |

Note:
This data was generated in placebo formulations.

TABLE 18E
Relative percentage of area and crystal count of spherulites
and Tmh crystals present in placebo formulations
prepared by different holding temperatures.

| | | | | | | |
|---|---|---|---|---|---|---|
| Holding T (° C.) | 48 | 52 | 54 | 56 | 58 | 66 |
| % Tmh crystals area | 92.5 | 98.7 | 100 | 100 | 71.9 | 0 |
| (relative to the area covered | | | | | | |
| by both Tmh crystals and | | | | | | |
| spherulites) | | | | | | |
| % Spherulites area | 7.5 | 1.3 | 0 | 0 | 28.1 | 100 |
| (relative to the area covered | | | | | | |
| by both Tmh crystals and | | | | | | |
| spherulites) | | | | | | |
| % Tmh crystals (crystal | 88.9 | 96.9 | 100 | 100 | 61.1 | 0 |
| count) | | | | | | |
| % Spherulites | 11.1 | 3.1 | 0 | 0 | 38.9 | 100 |
| (crystal count) | | | | | | |

TABLE 18F
Percentage of area, crystal count and average size of spherulites and Tmh
crystals present in formulations prepared by different holding temperatures.
Data in Table 18F was obtained from images taken at 50° C., or images taken
at 50° C. for samples stored at 5° C. (2-8 weeks), which were left for 1 hour at
room temperature prior to heating to 50° C. Results correspond to the image
analysis average from 3 replicates (standard deviation in parenthesis).

| Holding Temperature | 48° C. | 52° C. | 54° C. | 56° C. | 58° C. | 66° C. |
|---|---|---|---|---|---|---|
| % total Area of sample | 4.6 | 5.1 | 13.4 | 13.5 | 2.9 | NP |
| Tmh crystals | (0.4) | (2.3) | (7.7) | (3.5) | (1.1) | |
| Crystal count Tmh | 358.3 | 283.0 | 479.7 | 477.0 | 228.3 | NP |
| crystals | (11.7) | (90.1) | (179.5) | (212.1) | (161.3) | |
| Average size Tmh | 52.8 | 73.8 | 101.0 | 121.9 | 65.2 | NP |
| crystals (μm²) | (2.9) | (18.4) | (50.6) | (21.2) | (31.5) | |
| % total Area | 0.5 | 0.1 | NP | NP | 2.9 | 3.2 |
| spherulites | (0.1) | (0.1) | | | (1.1) | (1.6) |
| Crystal count | 63.7 | 14.7 | NP | NP | 344.7 | 453.3 |
| spherulites | (28.0) | (16.2) | | | (89.1) | (187.8) |
| Average size | 31.2 | 21.4* | NP | NP | 33.2 | 28.1 |
| spherulites (μm²) | (6.3) | (5.1) | | | (6.7) | (5.7) |

Average value of three measurements, with standard deviation in ( ).
*Average from 2 replicates because in 1 replicate, no spherulites were present.

TABLE 18G
Relative percentage by area and crystal count of spherulites
and Tmh crystals (averages of multiple samples)

| | | | | | | |
|---|---|---|---|---|---|---|
| Holding T (° C.) | 48 | 52 | 54 | 56 | 58 | 66 |
| % Tmh crystals area | 90.8 | 98.7 | 100 | 100 | 50.3 | 0 |
| (relative to the area | | | | | | |
| covered by both Tmh | | | | | | |
| crystals and spherulites) | | | | | | |
| % Spherulites (area) | 9.2 | 1.3 | 0 | 0 | 49.7 | 100 |
| (relative to the area | | | | | | |
| covered by both Tmh | | | | | | |
| crystals and spherulites) | | | | | | |
| % Tmh crystals (crystal | 84.9 | 95.1 | 100 | 100 | 39.8 | 0 |
| count) | | | | | | |
| % Spherulites | 15.1 | 4.9 | 0 | 0 | 60.2 | 100 |
| (crystal count) | | | | | | |

### Example 9

Formulations with or without a Retinoid (e.g., Adapalene) and/or a Tetracycline Antibiotic (e.g., Minocycline HCL) and Different Concentrations of Hydrogenated Castor Oil Prepared by a Continuous Heating-Cooling Process

This example shows that reduction in HCO amount in formulations prepared by a continuous heating-cooling process generally improves formulation fluidity but nevertheless does not result in fully shakable formulations.

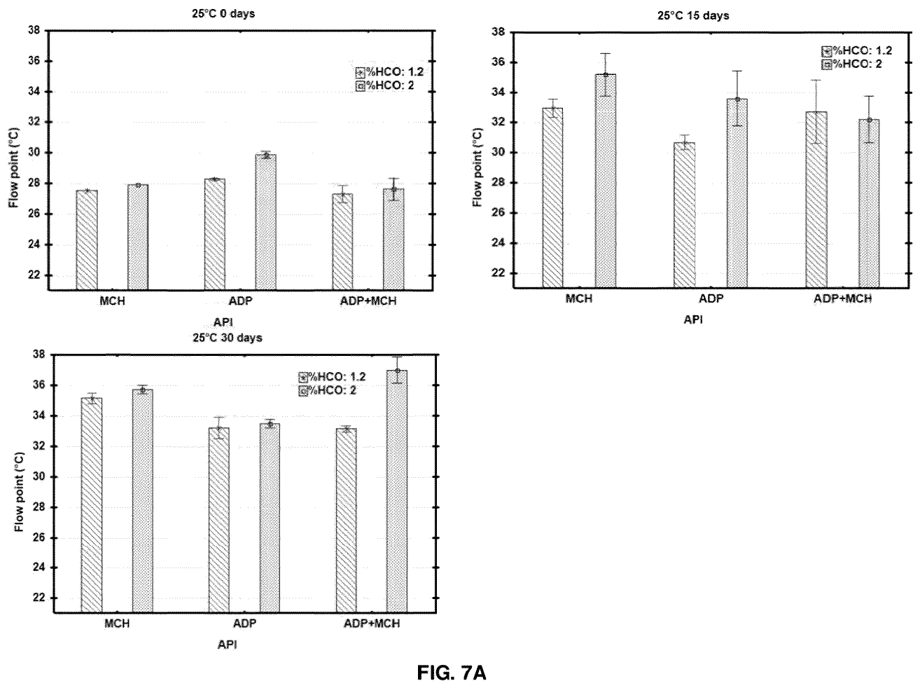

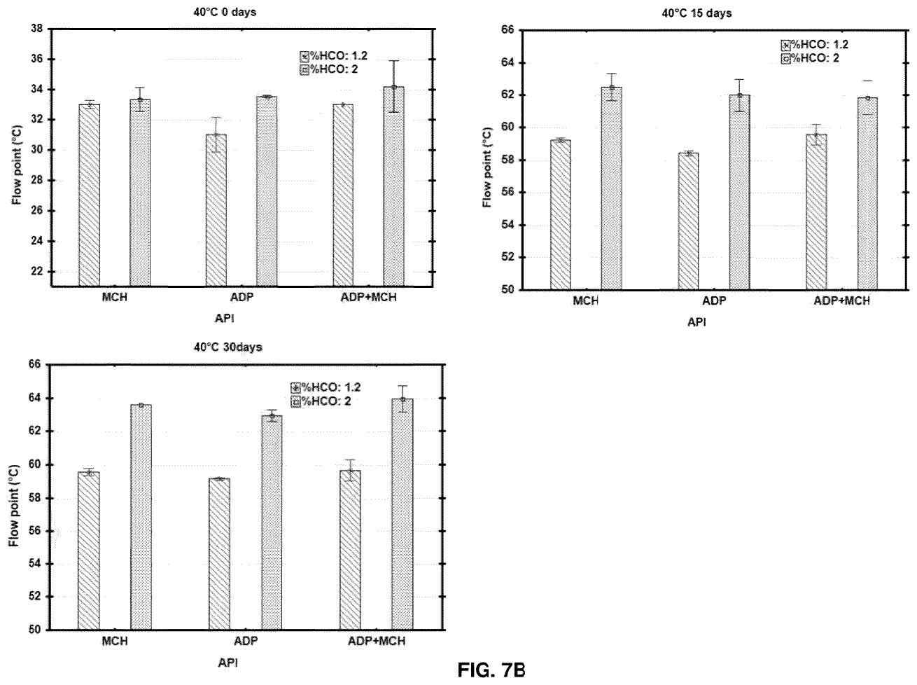

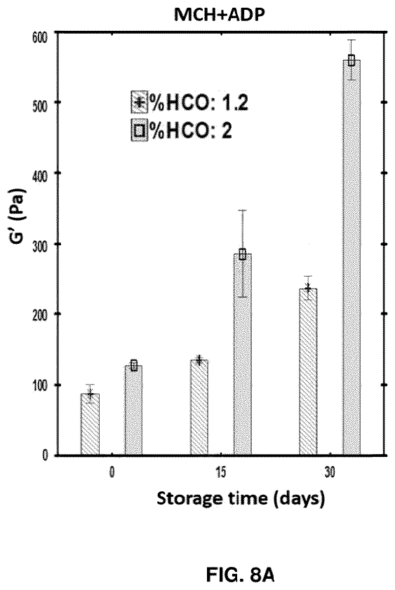

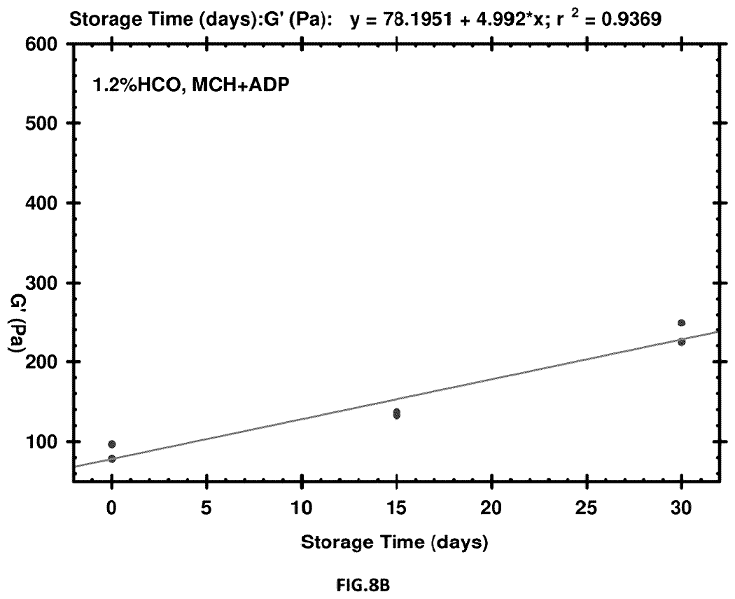

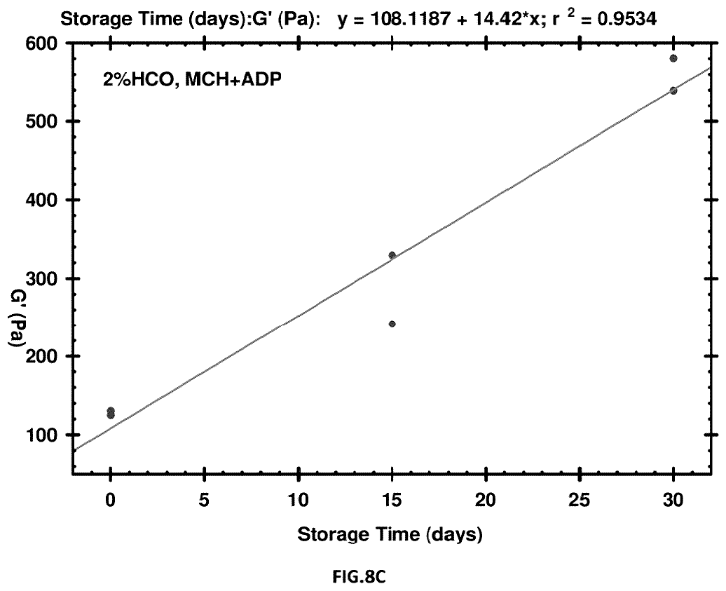

Formulations containing either 2% or 1.2% hydrogenated castor oil (HCO) and an active agent comprising either minocycline HCL alone (MCH), adapalene alone (ADP) or a combination of minocycline HCL and adapalene (MCH+ADP) were prepared by a continuous heating-cooling process as described in Example 1. As shown in Table 19A-C, FIG. 7A and FIG. 7B the flow point temperature, defined as the temperature where the system starts flowing, is generally higher for the samples with 2% HCO compared to samples with 1.2% HCO. Without being bound by any theory, a higher flow point temperature in formulations with 2% HCO may correlate with crystals that tend to interact with each other. These interactions over time can lead to crystal agglomeration and a less flowable formulation, which can lead to a reduction in shakability over time. It may also indicate that the microstructure in formulations comprising 2% HCO maintains a stronger tri-dimensional network that holds the liquid phase and retards the formulation from flowing as compared to formulations with 1.2% HCO, where the formulation starts flowing at a lower temperature. Without being bound by any theory, it could be that the HCO crystals act as nucleation points, based on which the other waxes in the formulation could aggregate. Therefore, higher amounts of HCO could lead to greater aggregation of solids in the formulation thereby reducing formulation flowability. In addition, as shown in Tables 11A-C and FIG. 8, the storage modulus G′, a rheological parameter that represents the resistance to being deformed elastically, was higher in formulations with 2% vs. 1.2% HCO comprising MCH+ADP kept at 25° C. at all time points tested, indicating the 2% HCO formulations were more rigid. The change in G′ over time in these formulations (FIGS. 8B-C) shows that the increase in G′ is steeper in formulations with 2% HCO vs. 1.2% HCO. According to the equations, after fitting, the rate of G′ change are 4.992 Pa/Day for 1.2% HCO, and 14.42 Pa/Day for 2% HCO, respectively.

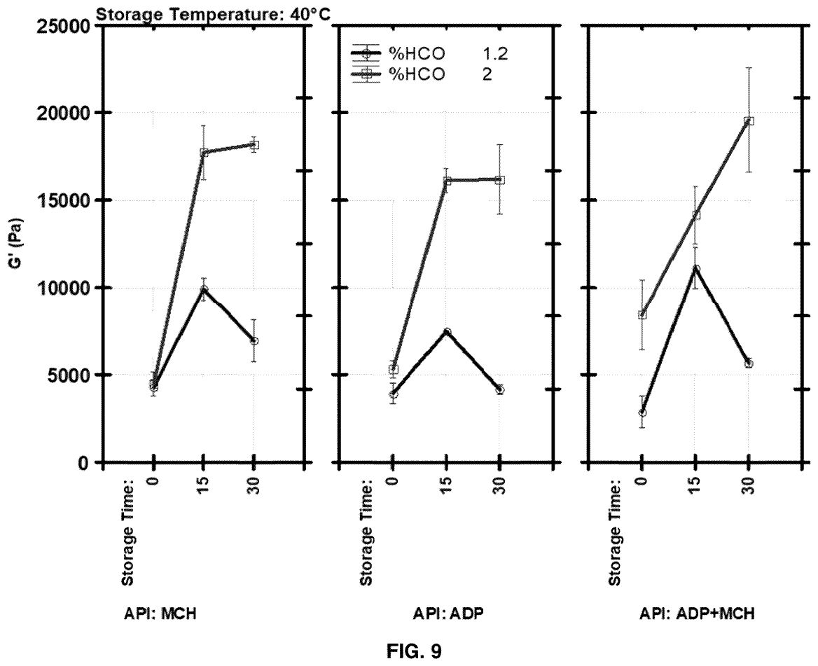

Interestingly, as can be seen in Tables 11 A-C and FIG. 9, G′ reached a plateau after 15 days in samples with 2% HCO kept at 40° C. containing either MCH alone or ADP alone. However, in a formulation with 2% HCO containing a combination of both MCH and ADP, G′ increased continuously at least until day 30 without reaching a plateau. The continuous increase in G′ is not likely to be explained by the increase in mass as the difference in solid mass between the MCH alone formulation and the MCH+ADP combination formulation was only 0.3 g, i.e. a less than 2% increase in solid mass. In other words, the continuous increase of G′ in the combination formulation is not explained solely by the addition of ADP. Instead, the continuous increase in G′ may be related to a synergistic effect created by the combination of MCH and ADP. Surprisingly, such a synergistic increase in G′ was not observed in formulations containing 1.2% HCO. Without being bound by any theory, the lower amount of HCO may, at least partly, “rescue” or hold back the formulation from the continuous increase in G′.

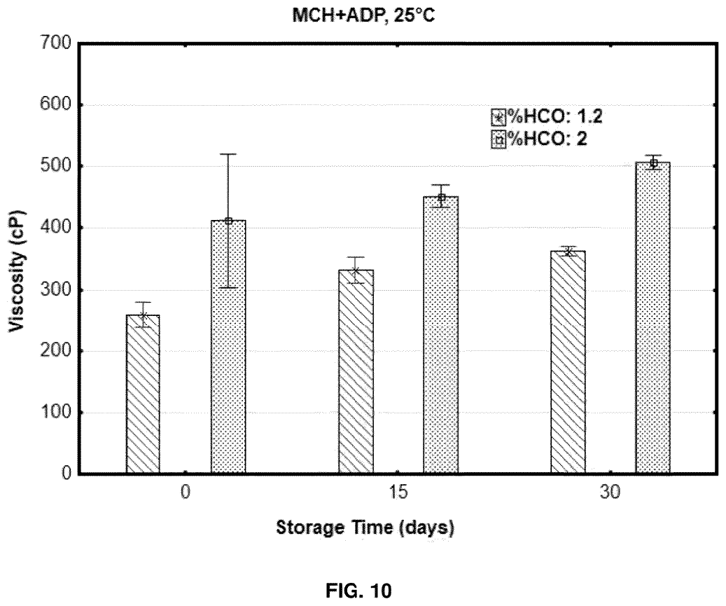

Viscosity is also higher for formulations with 2% HCO vs. 1.2% HCO, as shown in Tables 19A-C and FIG. 10.

As shown in Table 19, at Day 30, the combination formulation (MCH+ADP) was non-shakable when prepared with 2% HCO and moderately shakable when prepared with 1.2% HCO at 25° C. A marginal change in HCO concentration resulted in a change in shakability. The marginal reduction in HCO may contribute to improved shakability but may not allow for a fully shakable formulation. The marginal but consistent change in flow point between 2% HCO and 1.2% HCO may account for the change in shakability. The change in G′ over time in formulations with 2% HCO and 1.2% HCO may also account for the change in shakability. The marginal but consistent reduction in Flow point in formulations containing 1.2% HCO was insufficient to prevent blockage and allow for a fully shakable formulation. The reduction in G′ in formulations containing 1.2% HCO was insufficient in the continuous process to prevent blockage and allow for a fully shakable formulation.

TABLE 19A

| Study | | 1.1 | | | 1.2 | | |
|---|---|---|---|---|---|---|---|
| Formulation # | | 190313R | | | 190313N | | |
| HCO amount | | 2% | | | 2% | | |
| API | | MCH | | | MCH + ADP | | |
| Process | | Continuous heating-cooling | | | Continuous heating-cooling | | |
| | | process | | | process | | |
| Component | | | | | | | |
| Soybean oil | | 50 | | | 50 | | |
| coconut oil | | 23.6 | | | 23.60 | | |
| Light mineral oil* | | 2.8 | | | 2.5 | | |
| Stearic acid | | 3 | | | 3.00 | | |
| Docosanol | | 1.1 | | | 1.1 | | |
| Hydrogenated castor oil | | 2 | | | 2 | | |
| White wax (beeswax) | | 2 | | | 2 | | |
| Stearyl alcohol | | 1.5 | | | 1.5 | | |
| Cetostearyl alcohol | | 3.5 | | | 3.5 | | |
| Myristyl alcohol | | 2.5 | | | 2.5 | | |
| Cyclomethicone | | 5 | | | 5 | | |
| Minocycline HCl** | | 3 | | | 3 | | |
| Adapalene | | 0 | | | 0.30 | | |
| TOTAL | | 100 | | | 100 | | |
| | Temperature/ | | | | | | |
| Test | Time | T₀ | T₁₅ | T₃₀ | T₀ | T₁₅ | T₃₀ |
| Shakability | 5° C. | NA | NA | S | NA | NA | S |
| | 25° C. | NA | NA | N | NA | NA | N |
| | 40° C. | NA | NA | N | NA | NA | N |
| Rheology G′ | 5° C. | NA | 35.8 | 315.9 | NA | 89.1 | 450.1 |
| Pa | | | 0.5 | 17.3 | | 3.8 | 27.0 |
| | 25° C. | 137.9 | 377.9 | 514.2 | 127.5 | 285.7 | 560.1 |
| | | 30.5 | 11.8 | 32.5 | 3.8 | 61.7 | 28.7 |
| | 40° C. | 4461.7 | 17713.8 | 18179.4 | 8416.8 | 14139.6 | 19584.3 |
| | | 969.4 | 2189.8 | 616.0 | 2835.5 | 2336.3 | 4209.0 |
| Rheology | 5° C. | NA | <25 | 27.6 | NA | 26.9 | 27.4 |
| Flow Point, ° C. | | | | 0.1 | | 1.1 | 0.2 |
| | 25° C. | 27.9 | 35.2 | 35.7 | 27.6 | 32.2 | 37.0 |
| | | 0.0 | 1.4 | 0.3 | 0.7 | 1.6 | 0.8 |
| | 40° C. | 33.4 | 62.5 | 63.6 | 34.2 | 61.9 | 64.0 |
| | | 0.8 | 0.8 | 0.0 | 1.7 | 1.1 | 0.8 |
| Rheology | 5° C. | NA | 466.0 | 487.3 | NA | 388.5 | 503.3 |
| Viscosity, cP | | | 99.4 | 24.3 | | 42.9 | 26.9 |
| | 25° C. | 536.6 | 556.8 | 531.5 | 412.0 | 451.4 | 506.7 |
| | | 132.0 | 87.7 | 7.4 | 108.1 | 17.7 | 12.0 |
| | 40° C. | NA | NA | NA | NA | NA | NA |

TABLE 19B

| Study | | 1.3 | | | 1.4 | | |
|---|---|---|---|---|---|---|---|
| Formulation # | | 190317N | | | 190319H | | |
| HCO amount | | 2% | | | 1.2% | | |
| API | | ADP | | | MCH | | |
| Process | | Continuous heating-cooling | | | Continuous heating-cooling | | |
| | | process | | | process | | |
| Component | | | | | | | |
| Soybean oil | | 50 | | | 50 | | |
| coconut oil | | 23.6 | | | 23.6 | | |
| Light mineral oil* | | 5.5 | | | 3.6 | | |
| Stearic acid | | 3 | | | 3 | | |
| Docosanol | | 1.1 | | | 1.1 | | |
| Hydrogenated castor oil | | 2 | | | 1.2 | | |
| White wax (beeswax) | | 2 | | | 2 | | |
| Stearyl alcohol | | 1.5 | | | 1.5 | | |
| Cetostearyl alcohol | | 3.5 | | | 3.5 | | |
| Myristyl alcohol | | 2.5 | | | 2.5 | | |
| Cyclomethicone | | 5 | | | 5 | | |
| Minocycline HCl** | | 0 | | | 3 | | |
| Adapalene | | 0.3 | | | 0 | | |
| TOTAL | | 100 | | | 100 | | |
| | Temperature/ | | | | | | |
| Test | Time | T₀ | T₁₅ | T₃₀ | T₀ | T₁₅ | T₃₀ |
| Shakability | 5° C. | NA | NA | S | NA | NÅ | S |
| | 25° C. | NA | NA | S | NA | NA | S |
| | 40° C. | NA | NA | N | NA | NA | N |
| Rheology G′ | 5° C. | NA | 21.8 | 181.6 | NA | 34.7 | 290.2 |
| Pa | | | 0.6 | 18.2 | | 9.4 | 5.7 |
| | 25° C. | 220.7 | 204.2 | 314.6 | 74.9 | 177.7 | 306.2 |
| | | 155.6 | 22.0 | 116.3 | 36.0 | 14.7 | 35.6 |
| | 40° C. | 5308.3 | 16121.4 | 16191.6 | 4267.1 | 9915.4 | 6936.2 |
| | | 684.8 | 971.8 | 2806.9 | 651.6 | 887.4 | 1719.4 |
| Rheology | 5° C. | NA | <25 | 27.4 | NA | <25 | 26.7 |
| Flow Point, ° C. | | | | 0.1 | | | 0.1 |
| | 25° C. | 29.9 | 33.6 | 33.5 | 27.5 | 33.0 | 35.2 |
| | | 0.2 | 1.8 | 0.3 | 0.0 | 0.6 | 0.4 |
| | 40° C. | 33.6 | 62.0 | 63.0 | 33.0 | 59.2 | 59.6 |
| | | 0.1 | 1.0 | 0.4 | 0.3 | 0.1 | 0.2 |
| Rheology | 5° C. | NA | 299.6 | 363.5 | NA | 274.5 | 364.3 |
| Viscosity, cP | | | 8.6 | 1.0 | | 18.4 | 9.0 |
| | 25° C. | 238.8 | 406.3 | 509.2 | 223.3 | 321.5 | 359.6 |
| | | 27.9 | 6.1 | 49.1 | 5.9 | 7.0 | 8.5 |
| | 40° C. | NA | NA | NA | NA | NA | NA |

TABLE 19C

| Study | | 1.5 | | | 1.6 | | |
|---|---|---|---|---|---|---|---|
| Formulation # | | 190319S | | | 190317H | | |
| HCO amount | | 1.2% | | | 1.2% | | |
| API | | MCH + ADP | | | ADP | | |
| Process | | Continuous heating-cooling | | | Continuous heating-cooling | | |
| | | process | | | process | | |
| Component | | | | | | | |
| Soybean oil | | 50 | | | 50 | | |
| coconut oil | | 23.6 | | | 23.6 | | |
| Light mineral oil* | | 3.3 | | | 6.3 | | |
| Stearic acid | | 3 | | | 3 | | |
| Docosanol | | 1.1 | | | 1.1 | | |
| Hydrogenated castor oil | | 1.2 | | | 1.2 | | |
| White wax (beeswax) | | 2 | | | 2 | | |
| Stearyl alcohol | | 1.5 | | | 1.5 | | |
| Cetostearyl alcohol | | 3.5 | | | 3.5 | | |
| Myristyl alcohol | | 2.5 | | | 2.5 | | |
| Cyclomethicone | | 5 | | | 5 | | |
| Minocycline HCl** | | 3 | | | 0 | | |
| Adapalene | | 0.3 | | | 0.3 | | |
| TOTAL | | 100 | | | 100 | | |
| | Temperature/ | | | | | | |
| Test | Time | T₀ | T₁₅ | T₃₀ | T₀ | T₁₅ | T₃₀ |
| Shakability | 5° C. | NA | NA | S | NA | NA | S |
| | 25° C. | NA | NA | M | NA | NA | M |
| | 40° C. | NA | NA | N | NA | NA | N |
| Rheology G′ | 5° C. | NA | 124.1 | 325.3 | NA | 64.4 | 94.2 |
| Pa | | | 38.6 | 21.5 | | 2.6 | 33.1 |
| | 25° C. | 87.4 | 134.6 | 237.2 | 297.5 | 79.5 | 179.5 |
| | | 13.3 | 3.4 | 17.4 | 260.2 | 22.4 | 0.9 |
| | 40° C. | 2879.1 | 11136.2 | 5664.8 | 3929.2 | 7479.4 | 4148.9 |
| | | 1300.3 | 1674.0 | 387.5 | 821.0 | 41.9 | 377.2 |
| Rheology | 5° C. | NA | 26.5 | 26.8 | NA | 26.9 | 26.3 |
| Flow Point, ° C. | | | 0.1 | 0.3 | | 0.4 | 0.0 |
| | 25° C. | 27.3 | 32.7 | 33.2 | 28.3 | 30.7 | 33.2 |
| | | 0.6 | 2.1 | 0.2 | 0.1 | 0.5 | 0.7 |
| | 40° C. | 33.0 | 59.6 | 59.7 | 31.0 | 58.4 | 59.2 |
| | | 0.0 | 0.6 | 0.6 | 1.1 | 0.1 | 0.1 |
| Rheology | 5° C. | NA | 287.3 | 364.4 | NA | 268.2 | 277.9 |
| Viscosity, cP | | | 11.2 | 4.3 | | 9.0 | 2.6 |
| | 25° C. | 258.4 | 331.9 | 362.7 | 273.3 | 271.5 | 305.9 |
| | | 20.8 | 20.2 | 8.0 | 100.2 | 2.3 | 8.4 |
| | 40° C. | NA | NA | NA | NA | NA | NA |

*The amount of light mineral oil in the formulation is adjusted based on the amount of minocycline hydrochloride and/or adapalene.
**The amount of minocycline hydrochloride in the formulation is adjusted by the potency of the minocycline hydrochloride.

### Example 10. Isolation of Tmh Crystals

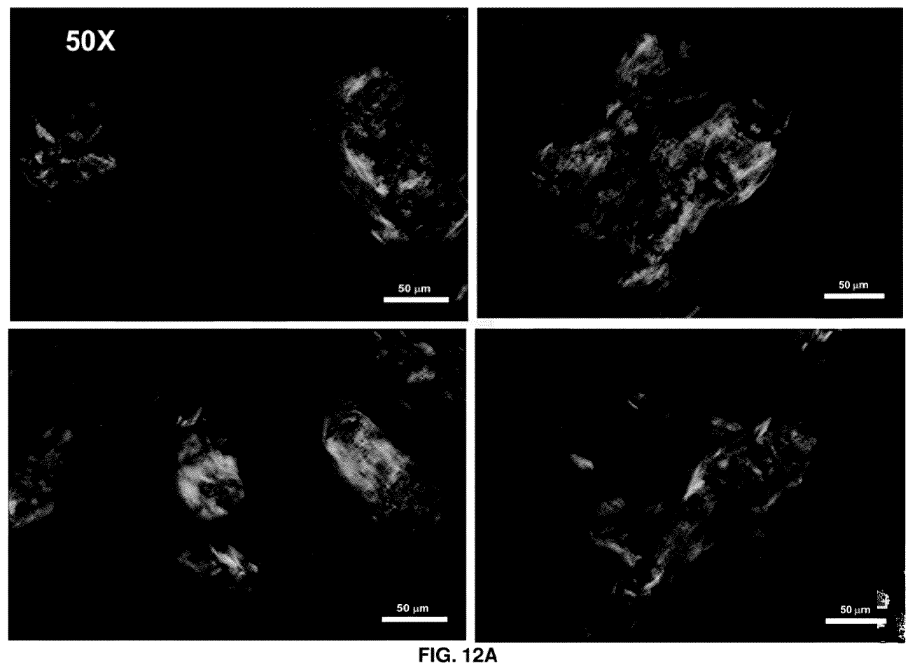

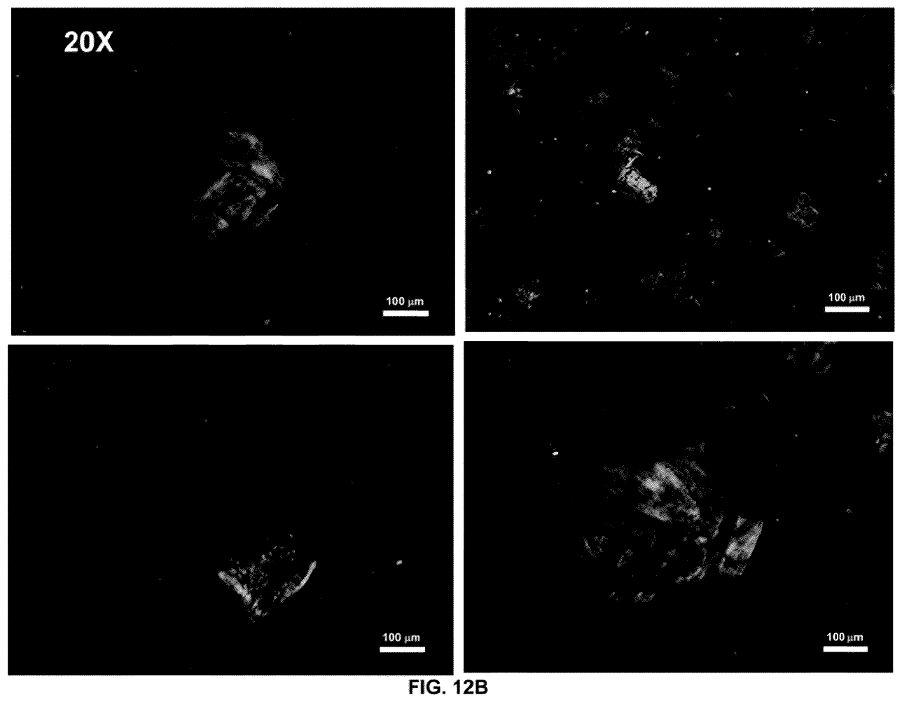

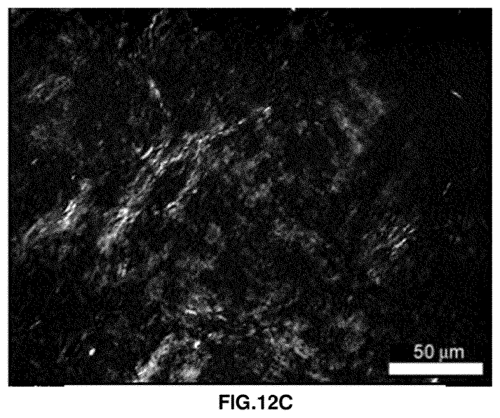

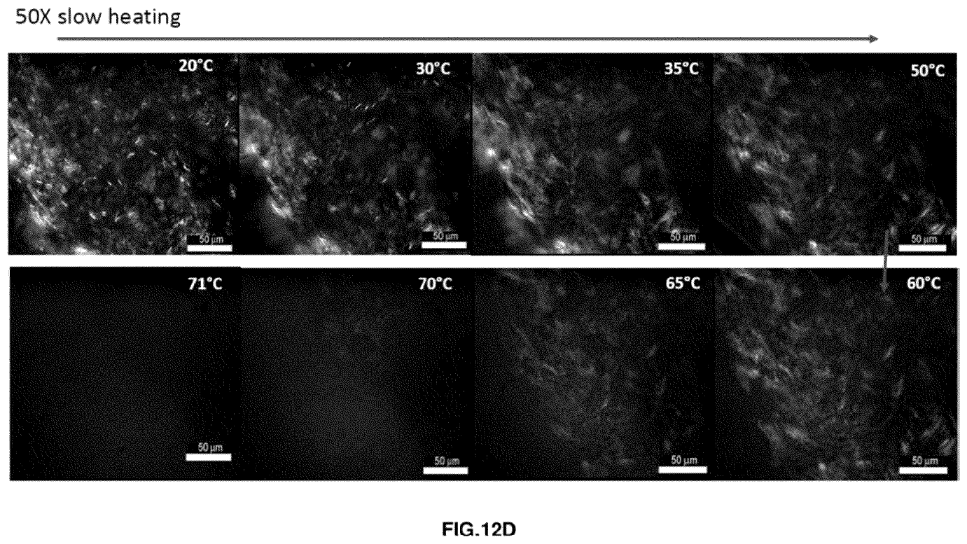

In an experiment to look for an underlying structure of the unit cell in Tmh crystals, the crystals were broken and isolated as described in the Method section. As shown in FIG. 12A-B some of the broken crystals appear approximately or generally tangled fibers.

Formulations comprising 1.2% HCO and 2% beeswax, prepared in a holding process were placed in either acetone or hexane (in order to separate between the crystals) and were microscopically analyzed as described in the Method section. FIG. 12C shows Tmh crystals in a formulation heated to 45° C. FIG. 12D shows Tmh crystals analyzed under slow heating (1° C./min). The non-uniform structures disappeared between 65° C.-70° C., Given the high melting temperature and that elsewhere it is seen that HCO has a significantly higher melting temperature in the formulation than other excipients these structures logically should correspond to HCO crystals.

### Example 11

Formulations with Different Waxes and/or Wax Ratios

This example evaluated the effect of different HCO:beeswax ratios and replacement of HCO by alternative waxes.

Formulations containing an active agent comprising a combination of minocycline HCL and adapalene (MCH+ADP) and different amounts of hydrogenated castor oil (HCO) and beeswax were evaluated (Tables 20a-b). In addition, formulations containing an active agent comprising a combination of minocycline HCl and adapalene (MCH+ADP), and alternative waxes such as paraffin wax or emulsifying wax instead of HCO were evaluated (Table 20c). The formulations were prepared by a holding process as described in Example 1.

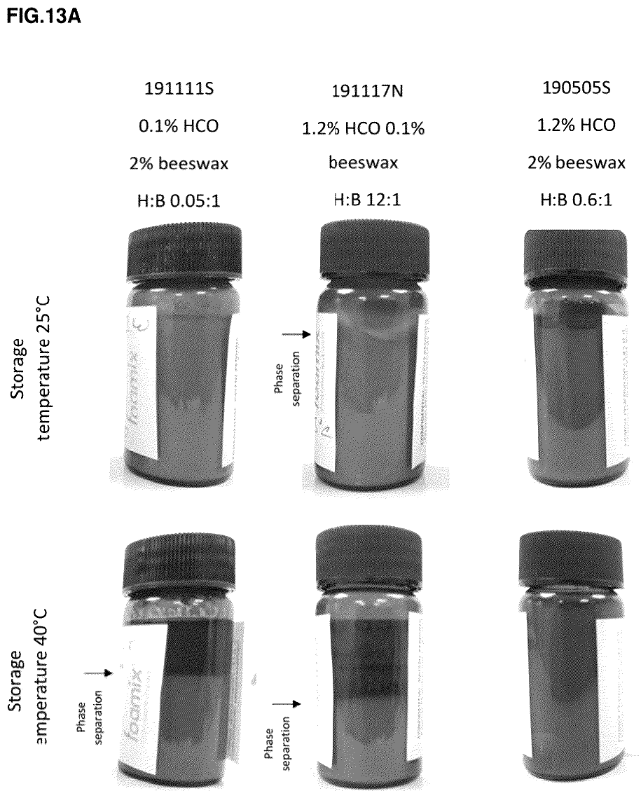

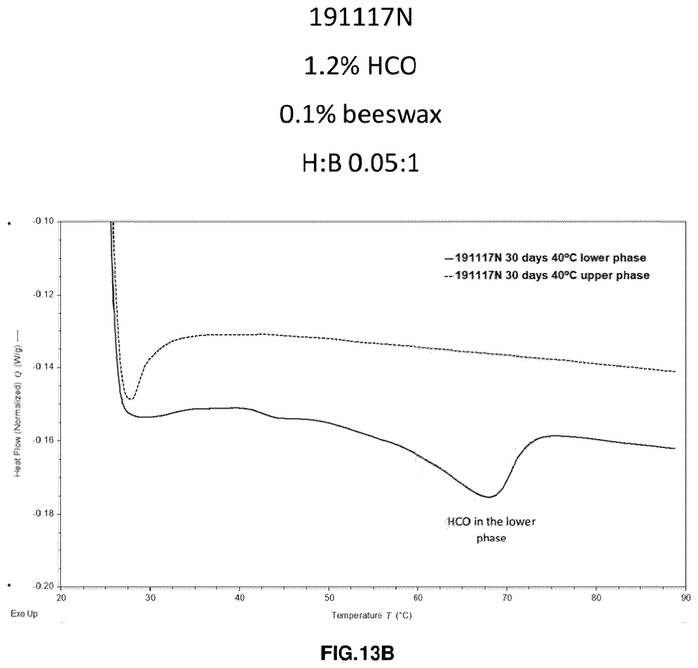

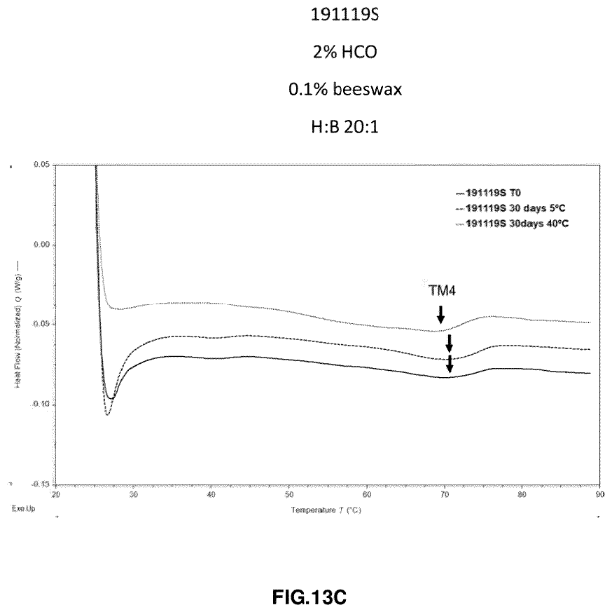

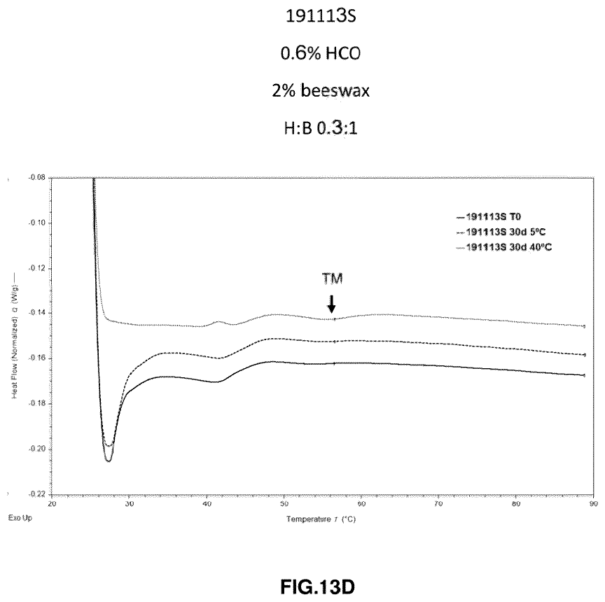

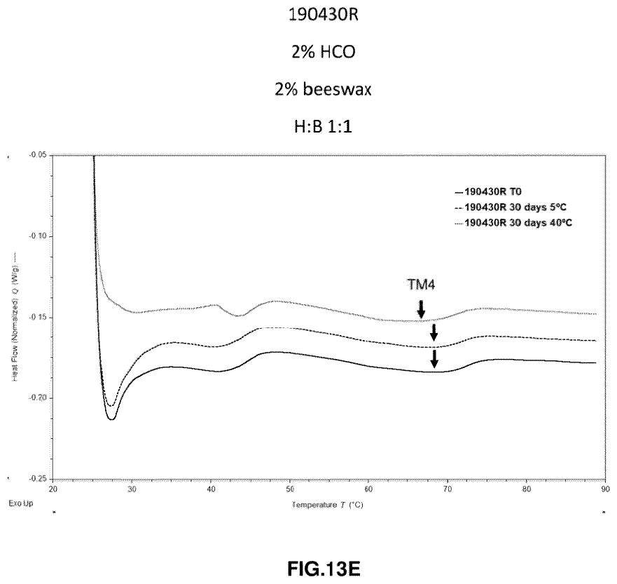

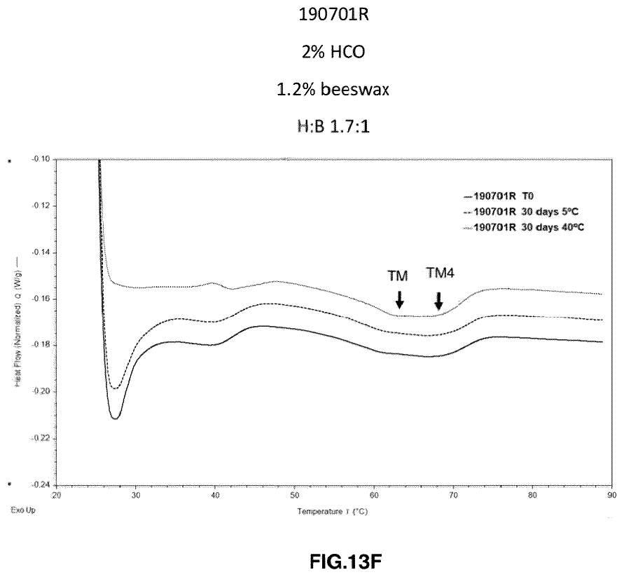

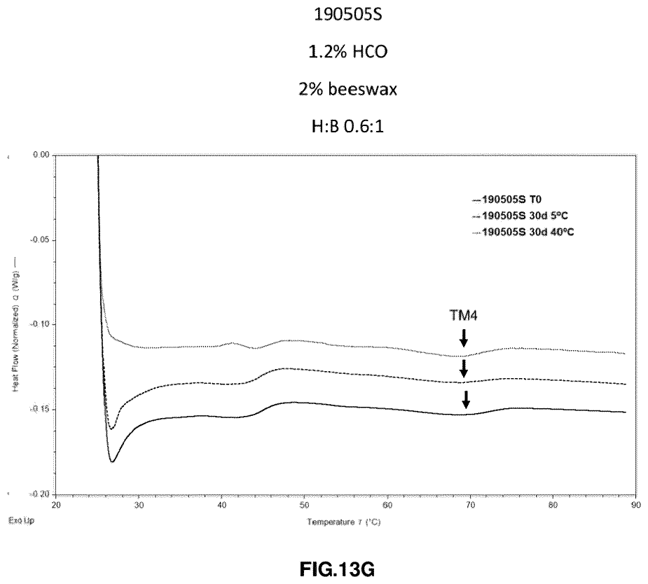

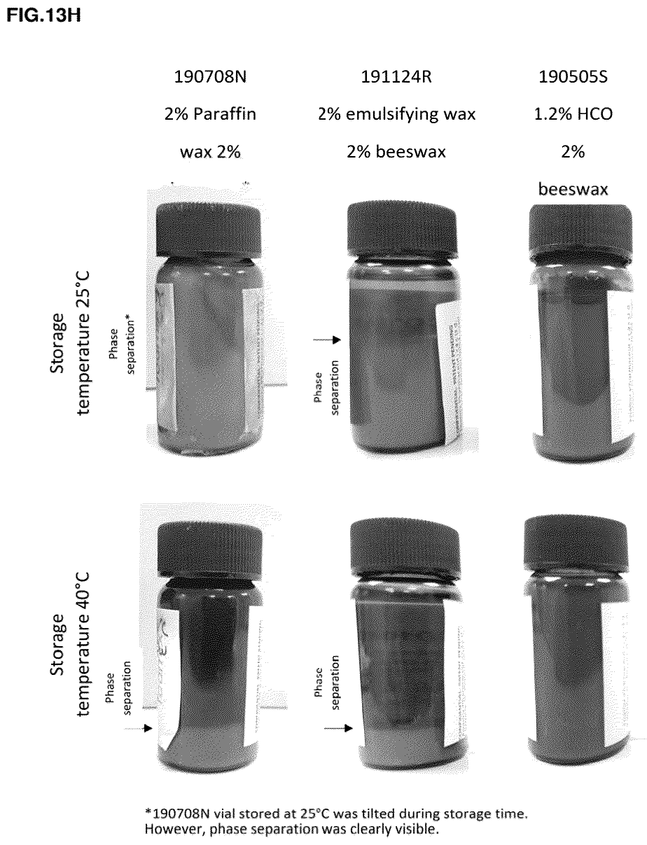

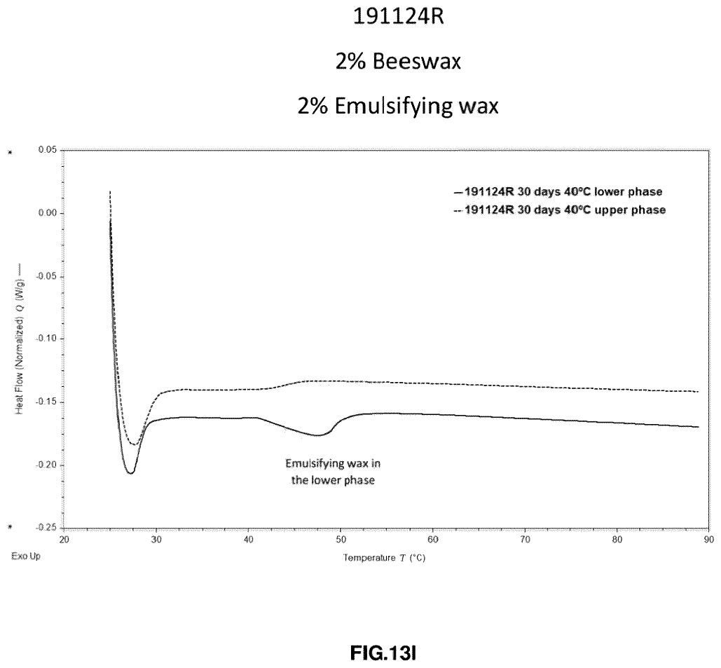

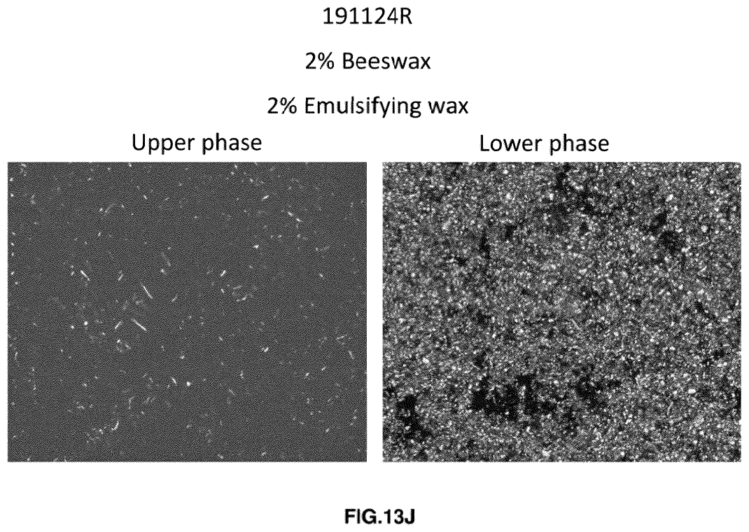

Formulation 191111S (Table 20a) containing 0.1% HCO and 2% beeswax, having a HCO:beeswax ratio of about 0.05:1 (1:20) was shakable on day 56 at all temperatures tested. However, for a formulation stored at 40° C., starting from day 30, phase separation and sedimentation of the active agent(s) was observed (FIG. 13A). This observation was hardly visible at 25° C. Without being bound by any theory, in a formulation stored at 40° C., the low amount of HCO was insufficient to form a matrix that holds the active agent(s) homogenously dispersed within the formulation. It is possible that in a formulation stored at 25° C., phase separation was prevented due to the presence of beeswax that is partially melted at about 40° C. (see Example 2). Without being bound by any theory, this result coupled with the other results shows that HCO in sufficient amounts can play an important role in forming a tri-dimensional matrix that can keep the formulation homogeneous and indicates that even low amounts, such as 0.1% can contribute and further it is possible that the presence of higher amounts of one or more other waxes and or increasing HCO (e.g., to about 0.25% or about 0.5% or more) may help prevent phase separation.

Formulation 191117N (Table 20b) containing 1.2% HCO and 0.1% beeswax, having a HCO:beeswax ratio of about 12:1 was shakable on day 56 at all temperatures tested. However, phase separation and sedimentation of the active agent(s) was observed, starting from day 30, in formulations stored at 25° C. and 40° C. (FIG. 13A). In addition, DSC analysis of the two fractions obtained in this formulation when stored at 40° C. for 30 days showed HCO was present only in the lower fraction of the formulation (FIG. 13B) Without being bound by any theory in formulations stored at both 25° C. and 40° C., the low amount of beeswax was insufficient to form the matrix that holds the active agent(s) homogenously dispersed within the formulation, even in the presence of HCO. The fact that phase separation was observed already in a formulation stored at 25° C., may indicate the important role of beeswax in preserving the matrix at this temperature. These results showed that both HCO and beeswax were involved in forming the matrix that held the active agent(s) in homogenous dispersion. Formulation 191119S (Table 20b) containing 2% HCO and 0.1% beeswax, having a HCO:beeswax ratio of about 20:1 was shakable when stored at 5° C. and 25° C. and moderately shakable at 40° C. on day 49. This formulation showed a high flow point temperature when stored at 40° C. on day 15 (59.7° C.) and day 30 (63.4° C.). Without being bound by any theory, a high flow point temperature represents a more rigid formulation and may indicate a future tendency to form a hard gel that ultimately might/could lead to formulation block. In addition, DSC analysis showed changes in DSC patterns between formulation stored at 5° C. to formulation stored at 40° C. TM4 seemed to be shifted to a lower temperature in a formulation stored at 40° C. for 30 days, Indicating formulation instability (FIG. 13C).

Formulation 191113S (Table 20b) containing 0.6% HCO and 2% beeswax, having a HCO:beeswax ratio of about 0.3:1 was shakable when stored at 5° C. and 25° C. and moderately shakable at 40° C. on day 56. This formulation showed a small increase in flow point temperature when stored at 40° C. on day 15 (56.8° C.) and day 30 (57.6° C.). These values were higher than the flow point for formulation 190505S (that showed optimal shakability and stability) but lower than the flow point for formulation 191119S. In addition, DSC analysis showed a transition around 55-56° C. that corresponded to the low amount of HCO. (FIG. 13D).

Formulation 190430R (Table 20a; 2% HCO, 2% beeswax, HCO:beeswax ratio of about 1:1) presented a relatively low flow point temperature when stored for 30 days at 40° C. (46.8° C.) and did not show phase separation or sedimentation of active agent(s) in any of the conditions tested. DSC analysis showed a slight shift in TM4 to a lower temperature in a formulation stored at 40° C. for 30 days. (FIG. 13E). Formulation was shakable when stored at 5° C. and 25° C., moderately shakable at 40° C. on day 15 and non-shakable at 40° C. on day 30.

Formulation 190701R (Table 20b; 2% HCO, 1.2% beeswax, HCO:beeswax ratio of about 1.7:1) presented a relatively low flow point temperature when stored for 30 days at 40° C. (41° C.) and did not show phase separation or sedimentation of active agent(s) in any of the conditions tested. DSC analysis showed the presence of TM4 and a slightly lower transition that were stable and showed no changes between formulations stored at 5° C. and at 40° C. on day 30 (FIG. 13F). Formulation was shakable when stored at 5° C. and 25° C. at all time points tested. When stored at 40° C., formulation was shakable on day 0 and day 15, and non-shakable on day 30.

A corresponding placebo formulation (Table 20b; 2% HCO, 1.2% beeswax, HCO:beeswax ratio of about 1.7:1) showed plates and very big (agglomerated) Tmh crystals. These crystals agglomerated with time especially when stored at 40° C. Phase separation was detected for samples stored at 40° C. from time 15 days-30 days. Additionally, the flow point for samples stored at 40° C. from 15 days were significantly high (68° C.). Without being bound by any theory, it could be that the active samples didn't present phase separation, high flow point and crystal agglomeration, due to the spatial interference of the actives that may delayed the agglomeration of the Tmh crystals.

Formulation 190505S (Table 20a; 1.2% HCO, 2% beeswax, HCO:beeswax ratio of about 0.6:1), showed optimal shakability. DSC analysis of this formulation showed minor changes between formulation stored at 5° C. and 40° C., indicating formulation stability. TM4 remained stable throughout storage time at all temperatures tested (FIG. 13G). This formulation did not present a high flow point temperature, phase separation or sedimentation of active agent(s) in any of the conditions tested. Altogether, these results indicated a unique combination of HCO and beeswax helped to maintain a matrix that held the active agent(s) in a homogenous suspension but at the same time keep the formulation flowable and shakable. The results provided in the multiple examples set out throughout the Examples section herein also indicate various ranges of concentrations and ratios of these substances at which they can be effective and illustrate their importance at specific concentrations and ratios.

Formulations comprising paraffin wax (Table 20c; 190708N) or emulsifying wax (Table 20c; 191124R) instead of HCO were shakable on all time points at all temperatures tested. However, phase separation and sedimentation of the active agent(s) was observed in both formulations (FIG. 13H). In addition, DSC analysis of the two fractions obtained in the 191124R formulation, showed a peak at about 48° C. that was present only in the lower fraction of the formulation indicating the presence of emulsifying wax in this fraction (FIG. 13I). Microscopic observation showed the presence of active agents only in this lower fraction (FIG. 13J). These results indicated HCO is important in forming the matrix that holds the active agent and was not replaced successfully in the Example (Table 20c), by paraffin wax or emulsifying wax. See Example 21 in which HCO is replaced with other waxes.

A corresponding placebo formulation comprising emulsifying wax instead of HCO (Table 20c) showed phase separation after stored at 25° C. for 5 months. Without being bound by any theory, the earlier phase separation in the active sample might be explained by the physical affinity (surface interaction) of the active ingredients to the emulsifying wax (since both were found in the lower layer) which may cause spatial interference of the emulsifying wax crystal-crystal network. Interference with such viscoelastic network may have resulted in the earlier observed phase separation.

TABLE 20a

| Study | | 7.4 | | | 7.5 | | | 3.9/7.3 | | | 3.4/7.2 | | |
|---|---|---|---|---|---|---|---|---|---|---|---|---|---|
| Formulation # | | 191111S | | | 191113S | | | 190505S | | | 190430R | | |
| HCO amount | | 0.1% | | | 0.6% | | | 1.2% | | | 2% | | |
| API | | MCH + ADP | | | MCH + ADP | | | MCH + ADP | | | MCH + ADP | | |
| Process | | Holding | | | Holding | | | Holding | | | Holding | | |
| Component | | | | | | | | | | | | | |
| Soybean oil | | 50 | | | 50 | | | 50 | | | 50 | | |
| coconut oil | | 23.6 | | | 23.6 | | | 23.6 | | | 23.6 | | |
| Light mineral oil* | | 4.4 | | | 3.9 | | | 3.3 | | | 2.5 | | |
| Stearic acid | | 3 | | | 3 | | | 3 | | | 3 | | |
| Docosanol | | 1.1 | | | 1.1 | | | 1.1 | | | 1.1 | | |
| Hydrogenated castor oil | | 0.1 | | | 0.6 | | | 1.2 | | | 2 | | |
| White wax (beeswax) | | 2 | | | 2 | | | 2 | | | 2 | | |
| Stearyl alcohol | | 1.5 | | | 1.5 | | | 1.5 | | | 1.5 | | |
| Cetostearyl alcohol | | 3.5 | | | 3.5 | | | 3.5 | | | 3.5 | | |
| Myristyl alcohol | | 2.5 | | | 2.5 | | | 2.5 | | | 2.5 | | |
| Cyclomethicone | | 5 | | | 5 | | | 5 | | | 5 | | |
| Minocycline HCl** | | 3 | | | 3 | | | 3 | | | 3 | | |
| Adapalene | | 0.3 | | | 0.3 | | | 0.3 | | | 0.3 | | |
| TOTAL | | 100 | | | 100 | | | 100 | | | 100 | | |
| Ratio HCO:Beeswax | | 0.05:1 | | | 0.3:1 | | | 0.6:1 | | | 1:1 | | |
| | Temperature/ | | | | | | | | | | | | |
| Test | Time | T0 | T15 | T30 | T0 | T15 | T30 | T0 | T15 | T30 | T0 | T15 | T30 |
| Shakability | 5° C. | N/A | N/A | S¥ | N/A | N/A | S¥ | S | S | S | S | S | S |
| | 25° C. | N/A | N/A | S¥ | N/A | N/A | S¥ | S | S | S | S | S | S |
| | 40° C. | N/A | N/A | S¥ | N/A | N/A | M¥ | S | M | M | S | M | N |
| Rheology | 5° C. | NA | 46 | 584 | NA | 67 | 327 | NA | 122.1 | 128.9 | NA | 134.0 | 247.3 |
| G′ Pa | | | 1.4 | 104.7 | | 34.9 | 66.0 | | 11.1 | 27.3 | | 21.8 | 17.8 |
| | 25° C. | 16 | 1860 | 2887 | 40 | 103 | 210 | 58.9 | 346.4 | 434.2 | 280.8 | 268.5 | 366.7 |
| | | 3.4 | 524.0 | 39.6 | 5.6 | 8.6 | 52.0 | 1.4 | 29.5 | 10.9 | 75.8 | 20.3 | 20.6 |
| | 40° C. | 16000 | 1232 | 1293 | 8350 | 4551 | 3841 | 12372.5 | 7884.6 | 5933.6 | 16381.0 | 5918.5 | 6294.7 |
| | | 0.0 | 427.8 | 65.1 | 307 | 574 | 817 | 482.2 | 1891.3 | 584.7 | 204.1 | 1857.2 | 580.8 |
| Rheology | 5° C. | NA | 25.9 | 27.2 | NA | 26.1 | 27.0 | NA | 26.4 | 26.7 | NA | 26.1 | 27.5 |
| Flow Point, | | | 0.71 | 0.28 | | 0.0 | 0.07 | | 0.8 | 1.1 | | 0.1 | 0.1 |
| ° C. | 25° C. | 23.3 | 28.5 | 28.7 | 26.8 | 30.8 | 34.3 | 24.9 | 34.6 | 36.3 | 27.1 | 32.6 | 35.7 |
| | | NA | 0.1 | 0.1 | 0.6 | 1.9 | 1.5 | 0.5 | 0.5 | 0.2 | 0.9 | 0.3 | 2.6 |
| | 40° C. | 33.5 | 43.7 | 47.4 | 32.7 | 56.8 | 57.6 | 32.2 | 53.65 | 54.85 | 33.3 | 43.7 | 46.8 |
| | | 0.5 | 6.2 | 0.0 | 0.0 | 0.1 | 0.3 | 0.0 | 0.5 | 1.3 | 0.4 | 1.2 | 2.4 |
| Rheology | 5° C. | NA | 169.9 | 260.9 | NA | 228.8 | 317.6 | NA | 244.5β | 291.6 β | NA | 380.0 | 401.8 |
| Viscosity, cP | | | NA | NA | | NA | NA | | | | | 0.0 | 0.0 |
| | 25° C. | NA | 418.8$ | 613.0$ | NA | 246.2 | 318.8 | 251.1β | NA | 368.9 β | 303.6 | 416.3 | 472.5 |
| | | | NA | NA | | NA | NA | | | | 0.0 | 0.0 | 0.0 |
| | 40° C. | NA | NA | NA | | | NA | NA | NA | NA | NA | NA | NA |
| DSC TM2 | 5° C. | NA | 27.40 | 27.2 | NA | 27.6 | 27.3 | NA | 27.1 | 26.7 | NA | 27.4 | 27.2 |
| | | | 0.1 | 0.2 | | 0.0 | 0.1 | | 0.1 | 0.1 | | 0.0 | 0.6 |
| | 25° C. | 27.2 | 27.6 | 27.7 | 27.4 | 27.2 | 27.5 | 26.9 | 27.1 | 27.0 | 27.2 | 27.3 | 27.4 |
| | | NA | NA | 0.07 | 0.07 | 0.07 | 0.07 | 0.1 | 0.1 | 0.1 | 0.7 | 0.3 | 0.4 |
| | 40° C. | 27.2 | 27.4 | 28.2 | 27.0 | 27.1 | | 26.5 | | | 26.7 | 27.3 | |
| | | 0.5 | 0.1 | NA | 0.1 | 0.1 | | 0.0 | | | 0.1 | 0.2 | |
| DSC TM2.1 | 5° C. | | | 34.8 | NA | | | NA | | | NA | | |
| | | | | 0.2 | | | | | | | | | |
| | 25° C. | | | | | | | | | | | | |
| | 40° C. | | | | 32.0 | | | 31.9 | 29.2 | 30.3 | | | 29.7 |
| | | | | | | | | 0.1 | 2.0 | 0.3 | | | 0.6 |
| DSC TM3.1 | 5° C. | | | | | | 35.6 | NA | | | NA | | |
| | | | | | | | 0.07 | | | | | | |
| | 25° C. | | 40.1 | | | 35.2 | | | | | | | |
| | | | NA | | | 0.28 | | | | | | | |
| | 40° C. | 40.2 | 39.0 | 39.7 | 40.5 | 39.8 | 40.0 | 39.9 | 38.3 | 38.8 | 39.7 | 38.0 | 37.7 |
| | | 0.0 | 0.4 | 0.1 | 0.0 | 0.1 | 0.07 | 0.3 | 0.1 | 0.3 | 0.3 | 0.6 | 0.4 |
| DSC TM3 | 5° C. | NA | 44.0 | 44.2 | NA | 42.2 | 41.8 | NA | 42.4 | 42.1 | NA | 42.6 | 41.4 |
| | | | 0.5 | 0.4 | | 0.1 | 0.5 | | 0.1 | 0.1 | | 0.0 | 0.2 |
| | 25° C. | 44.3 | 43.5 | 43.0 | 42.0 | 42.3 | 42.2 | 42.1 | 42.3 | 41.9 | 41.9 | 41.4 | 41.6 |
| | | NA | NA | 0.3 | 0.4 | 0.2 | 0.9 | 0.2 | 0.7 | 0.2 | 0.3 | 0.5 | 0.1 |
| | 40° C. | 44.0 | 44.1 | 45.2 | 42.6 | 43.4 | 43.3 | 43.5 | 43.8 | 44.1 | 43.5 | 43.7 | 43.5 |
| | | 0.4 | 0.1 | 0.0 | 0.0 | 0.4 | 0.1 | 0.1 | 0.1 | 0.2 | 0.0 | 0.1 | 0.2 |
| DSC TM4.2 | 5° C. | | | | NA | 55.1 | 54.1 | NA | | | NA | | |
| | | | | | | 0.2 | 0.0 | | | | | | |
| | 25° C. | | | | 55.3 | 55.2 | 55.5 | | | | | | |
| | | | | | NA | 0.4 | 0.3 | | | | | | |
| | 40° C. | | | | 54.4 | 56.2 | 56.7 | | 53.7 | 56.1 | | | |
| | | | | | 0.8 | 0.6 | 0.1 | | 0.1 | 0.5 | | | |
| DSC TM4.1 | 5° C. | | | | | | | NA | | | NA | | |
| | 25° C. | | | | | | | | | | | | |
| | 40° C. | | | | | | | | | | | 60.6 | 62.7 |
| | | | | | | | | | | | | 0.1 | 0.3 |
| DSC TM4 | 5° C. | | | | | | | NA | 68.5 | 68.6 | NA | 68.6 | 67.7 |
| | | | | | | | | | 0.4 | 0.2 | | 0.14 | 0.35 |
| | 25° C. | | | | | | | 69.3 | 69.2 | 68.8 | 68.2 | 68.4 | 68.2 |
| | | | | | | | | 0.8 | 0.2 | 0.5 | 0.28 | 0.21 | 0.49 |
| | 40° C. | | | | | | | 69.4 | 68.4 | 68.6 | 67.9 | 67.3 | 66.3 |
| | | | | | | | | 0.3 | 0.2 | 0.4 | 0.21 | 0.28 | 0.14 |

*The amount of light mineral oil in the formulation is adjusted based on the amount of minocycline hydrochloride and/or adapalene.
**The amount of minocycline hydrochloride in the formulation is adjusted by the potency of the minocycline hydrochloride.
$Separated into two phases. Rheometry presented for lower phase only.
¥Shakability was evaluated on day 56.
βOnly one replicate was performed

TABLE 20b

| Study | | | | | 7.1 | | | 7.6 | | | 7.7 | | |
|---|---|---|---|---|---|---|---|---|---|---|---|---|---|
| Formulation # | | Placebo (2%HCO; | | | 190701R | | | 191117N | | | 191119S | | |
| | | 1.2% beeswax) | | | | | | | | | | | |
| HCO amount | | 2% | | | 2% | | | 1.2% | | | 2% | | |
| API | | Placebo | | | MCH + ADP | | | MCH + ADP | | | MCH + ADP | | |
| Process | | Holding | | | Holding | | | Holding | | | Holding | | |
| Component | | | | | | | | | | | | | |
| Soybean oil | | 50 | | | 50 | | | 50 | | | 50 | | |
| coconut oil | | 23.6 | | | 23.6 | | | 23.6 | | | 23.6 | | |
| Light mineral oil* | | 6.6 | | | 3.3 | | | 5.2 | | | 4.4 | | |
| Stearic acid | | 3 | | | 3 | | | 3 | | | 3 | | |
| Docosanol | | 1.1 | | | 1.1 | | | 1.1 | | | 1.1 | | |
| Hydrogenated castor oil | | 2 | | | 2 | | | 1.2 | | | 2 | | |
| White wax (beeswax) | | 1.2 | | | 1.2 | | | 0.1 | | | 0.1 | | |
| Stearyl alcohol | | 1.5 | | | 1.5 | | | 1.5 | | | 1.5 | | |
| Cetostearyl alcohol | | 3.5 | | | 3.5 | | | 3.5 | | | 3.5 | | |
| Myristyl alcohol | | 2.5 | | | 2.5 | | | 2.5 | | | 2.5 | | |
| Cyclomethicone | | 5 | | | 5 | | | 5 | | | 5 | | |
| Minocycline HCl** | | 0 | | | 3 | | | 3 | | | 3 | | |
| Adapalene | | 0 | | | 0.3 | | | 0.3 | | | 0.3 | | |
| TOTAL | | 100 | | | 100 | | | 100 | | | 100 | | |
| Ratio HCO:Beeswax | | 1.7:1 | | | 1.7:1 | | | 12:1 | | | 20:1 | | |
| | Temperature/ | | | | | | | | | | | | |
| Test | Time | T0 | T15 | T30 | T0 | T15 | T30 | T0 | T15 | T30 | T0 | T15 | T30 |
| Shakability | 5° C. | | | | S | S | S | | | S¥ | N/A | N/A | S# |
| | 25° C. | | | | S | S | S | | | S¥ | N/A | N/A | S# |
| | 40° C. | | | | S | S | N | | | S¥ | N/A | N/A | M# |
| Rheology G′ Pa | 5° C. | NA | 53.3 | 41.1 | NA | 101 | 198 | NA | 27 | 49 | NA | NA | 15089 |
| | | | 15.8 | 24.3 | | | | | 2.8 | 4.9 | | | |
| | 25° C. | 34.7 | 254.4 | 285.6 | 90 | 139.5 | 194 | 9 | 100 | 600 | 25 | 75 | 1902 |
| | | 1.4 | 91.3 | 154.8 | | | | 1.0 | 11.0 | 160.5 | 12.7 | 14.1 | 290.6 |
| | 40° C. | 4897.9 | 18378.2 | 15344.6 | 13470 | 2155 | 2633 | 355 | 133 | 69$$ | 1066 | 4264 | 4385 |
| | | 273.8 | 781.4 | 1962.4 | | | | 146.4 | 22.6 | NA | 304.8 | 699.0 | 103.2 |
| | | | | | | | | | | 900 | | | |
| | | | | | | | | | | NA | | | |
| Rheology | 5° C. | NA | 27.6 | 26.8 | NA | 29.7 | 26.6 | NA | 25.4 | 25.4 | NA | NA | 27.1 |
| Flow Point, ° C. | | | 0.0 | 0.3 | | | | | 0.57 | 0.64 | | | 1.13 |
| | 25° C. | 28.3 | 32.7 | 36.9 | 26.8 | 26.7 | 31.7 | 23.0 | 31.4 | 36.4 | <22 | 25.9 | 33.7 |
| | | 0.2 | 3.2 | 1.4 | | | | 1.41 | 0.78 | 1.01 | NA | NA | 1.77 |
| | 40° C. | 34.5 | 68.8 | 68.8 | 33.7 | 39.5 | 41.0 | 24.7 | 30.1 | 28.5$$ | 29.1 | 59.7 | 63.4 |
| | | 0.4 | 0.2 | 0.7 | | | | 1.77 | 0.57 | NA | 0.64 | 0.28 | 0.14 |
| | | | | | | | | | | 39 | | | |
| | | | | | | | | | | NA | | | |
| Rheology Viscosity, | 5° C. | NA | 245.8 | 352.8 | NA | 315.6 | 366.7 | NA | 172.2 | 189.4 | NA | NA | 325.5 |
| cP | | | 0.5 | 12.8 | | NA | NA | | NA | NA | | | NA |
| | 25° C. | 216.8 | 330.1 | 347.2 | 301.2 | 321.8 | 352.4 | NA | 204.3 | 308.8 | NA | 292.8 | 649.8 |
| | | 0.6 | 1.5 | 11.8 | NA | NA | NA | | NA | NA | | NA | NA |
| | 40° C. | NA | NA | NA | NA | NA | NA | | NA | NA | | | |
| DSC TM2 | 5° C. | NA | 26.6 | 26.7 | NA | 27.7 | 27.4 | NA | 27.4 | 27.0 | NA | NA | 26.7 |
| | | | 0.1 | 0.1 | | 0.0 | 0.0 | | 0.71 | 0.07 | | | 0.0 |
| | 25° C. | 27.7 | 26.6 | 26.5 | 27.6 | 27.4 | 27.2 | 27.3 | 27.7 | 28.1 | 27.3 | 27.6 | 28.2 |
| | | 0.2 | 0.2 | 0.1 | 0.1 | 0.1 | 0.2 | 0.1 | 0.0 | 0.3 | 0.1 | 0.3 | 0.1 |
| | 40° C. | 27.1 | 27.0 | | 27.2 | 27.2 | 27.7 | 26.7 | 27.4 | 27.78 | 26.9 | 27.9 | 28.4 |
| | | 0.2 | 0.1 | | 0.1 | 0.1 | NA | 0.2 | 0.3 | 0.2 | 0.2 | 0.6 | 0.0 |
| DSC TM2.1 | 5° C. | NA | 30.0 | 29.5 | | | | | | | NA | NA | |
| | | | 0.1 | 0.1 | | | | | | | | | |
| | 25° C. | 32.6@ | | 32.0@ | | | | | | | | | |
| | 40° C. | | | 31.7 | | | | | | | | | |
| | | | | 0.6 | | | | | | | | | |
| DSC TM3.1 | 5° C. | NA | | | NA | | | | | | NA | NA | 40.5 |
| | | | | | | | | | | | | | 0.4 |
| | 25° C. | | | | | | | | 39.0 | 39.5 | 41.1 | 40.8 | 40.6 |
| | | | | | | | | | 0.5 | 0.6 | 0.6 | 0.2 | 0.2 |
| | 40° C. | 39.4 | | | 39.8 | 37.1 | 38.0 | | 39.2 | | | | |
| | | 0.2 | | | 0.1 | 0.0 | 0.0 | | 0.42 | | | | |
| DSC TM3 | 5° C. | NA | 39.4 | 39.7 | NA | 42.1 | 40.5 | | | | NA | NA | |
| | | | 0.3 | 0.3 | | NA | 0.1 | | | | | | |
| | 25° C. | 40.0 | 40.1 | 40.9 | 40.1 | 41.7 | 40.5 | | | | | | |
| | | 0.0 | 0.1 | 0.1 | 0.4 | 0.1 | 0.3 | | | | | | |
| | 40° C. | 43.7 | 41.7 | 43.0 | 43.5 | 45.6 | 42.1 | | | 43.0$ | 43.7 | 43.9 | 44.0 |
| | | 0.1 | 0.3 | 1.0 | 0.2 | 0.1 | 0.1 | | | 1.0 | 0.0 | 0.2 | 0.4 |
| DSC TM4.2 | 5° C. | NA | | | | | | | | | NA | NA | NA |
| | 25° C. | | | | | | | | | | | | |
| | 40° C. | 53.9@ | | | | | | | | | | 56.2 | 56.2 |
| | | | | | | | | | | | | 0.3 | 0.2 |
| DSC TM4.1 | 5° C. | NA | | | NA | 60.8 | 60.9 | | | | NA | | |
| | | | | | | 0.4 | 0.2 | | | | | | |
| | 25° C. | | | | 62.9 | 62.8 | 61.5 | | | | | | |
| | | | | | 0.1 | 0.1 | 0.4 | | | | | | |
| | 40° C. | | 60.2 | 60.5 | 60.8 | 62.2 | 62.9 | | | | | | |
| | | | 0.7 | 0.1 | 0.07 | 0.1 | 0.6 | | | | | | |
| DSC TM4 | 5° C. | NA | 71.8 | 71.9 | NA | 67.1 | 67.0 | NA | 66.4 | 66.6 | NA | NA | 70.3 |
| | | | 0.1 | 0.1 | | 0.0 | 0.1 | | 0.1 | 0.2 | | | 0.2 |
| | 25° C. | 71.4 | 72.0 | 72.1 | 67.1 | 67.3 | 67.8 | 67.1 | 66.8 | 67.0 | 69.9 | 69.8 | 70.2 |
| | | 0.8 | 0.3 | 0.0 | 0.0 | 0.4 | NA | 0.2 | 0.1 | 0.0 | 0.4 | 0.3 | 0.0 |
| | 40° C. | 71.7 | 71.4 | 71.6 | 67.3 | 66.0 | 68.5 | 66.3 | 66.4 | 68.0$ | 70.3 | 69.5 | 68.4 |
| | | 0.4 | 0.0 | 0.1 | 0.3 | 0.1 | 0.8 | 0.9 | 0.5 | 0.1 | 0.1 | 0.2 | 0.45 |

*The amount of light mineral oil in the formulation is adjusted based on the amount of minocycline hydrochloride and/or adapalene.
**The amount of minocycline hydrochloride in the formulation is adjusted by the potency of the minocycline hydrochloride.
$Separated into two phases. TM3 and TM4 observed in lower phase only.
$$Separated into two phases. Result obtained for lower phase presented below the result for upper phase.
&TM2 observed in upper phase only
#Shakability was evaluated on day 49.
¥ Shakability was evaluated on day 56.
@Parameter present in only one replicate.

TABLE 20c

| Study | | 6.1 | | | 6.2 | | | | | |
|---|---|---|---|---|---|---|---|---|---|---|
| Formulation # | | 190708N | | | 191124R | | | | | |
| API | | MCH + ADP | | | MCH + ADP | | | Placebo (emulsifying | | |
| | | | | | | | | wax) | | |
| Process | | Holding | | | Holding | | | Holding | | |
| Component | | | | | | | | | | |
| Soybean oil | | 50 | | | 50 | | | 50 | | |
| coconut oil | | 23.6 | | | 23.6 | | | 23.6 | | |
| Light mineral oil* | | 2.5 | | | 2.5 | | | 5.8 | | |
| Stearic acid | | 3 | | | 3 | | | 3 | | |
| Docosanol | | 1.1 | | | 1.1 | | | 1.1 | | |
| Paraffin wax | | 2 | | | 0 | | | 0 | | |
| Emulsifying wax | | 0 | | | 2 | | | 2 | | |
| White wax (beeswax) | | 2 | | | 2 | | | 2 | | |
| Stearyl alcohol | | 1.5 | | | 1.5 | | | 1.5 | | |
| Cetostearyl alcohol | | 3.5 | | | 3.5 | | | 3.5 | | |
| Myristyl alcohol | | 2.5 | | | 2.5 | | | 2.5 | | |
| Cyclomethicone | | 5 | | | 5 | | | 5 | | |
| Minocycline HCl** | | 3 | | | 3 | | | 0 | | |
| Adapalene | | 0.3 | | | 0.3 | | | 0 | | |
| TOTAL | | 100 | | | 100 | | | 100 | | |
| | Temperature/ | | | | | | | | | |
| Test | Time | T0 | T0 | T0 | T0 | T15 | T30 | T0 | T15 | T30 |
| Shakability | 5° C. | S | S | S | S | NA | S | | | |
| | 25° C. | S | S | S | S | NA | S | | | |
| | 40° C. | S | S | S | S | NA | S | | | |
| Rheology G′ Pa | 5° C. | NA | 331 | 87 | NA | 8 | 7 | NA | 2.9 | 7.5 |
| | | | 79.9 | 4.1 | | 4.0 | 2.5 | | 1.0 | 1.4 |
| | 25° C. | 44 | 110 | 71 | 16 | 64 | 53 | 2.8 | 7.3 | 14.7 |
| | | 3.0 | 2.8 | 12.0 | 10.7 | 7.1 | 4.2 | 0.3 | 2.2 | 9.6 |
| | 40° C. | 16928 | 5253 | 2620 | >105 | 1241$ | 933$$ | 11518.2 | 138.7 | 13.0 |
| | | NA | 1339.3 | 619.2 | | 96.2 | NA | 2380.5 | 46.6 | 1.1 |
| | | | | | | | 1537 | | | |
| | | | | | | | NA | | | |
| Rheology | 5° C. | NA | 27.7 | 28,1 | NA | 24.1 | <22 | NA | <25 | <25 |
| Flow Point, ° C. | | | 0.78 | 0.42 | | 0.07 | NA | | | |
| | 25° C. | 27.9 | 28.5 | 27.9 | 22.9 | 24.9 | 23 | <25 | <25 | <25 |
| | | 0.4 | 0.8 | 0.3 | 0.4 | 0.6 | NA | | | |
| | 40° C. | 43.0 | 36.0 | 34.8 | 35.0 | 30.2$ | 25.9$$ | 34.8 | 27.5 | <25 |
| | | NA | 0.6 | 0.7 | 1.6 | NA | NA | 0.5 | 0.0 | |
| | | | | | | | 29.6 | | | |
| | | | | | | | NA | | | |
| Rheology Viscosity, cP | 5° C. | NA | 297.8 | 370.8 | NA | 137.6 | 160.2 | NA | 123.2 | 126.7 |
| | | | NA | NA | | NA | NA | | 0.3 | 6.6 |
| | 25° C. | 166.7 | 205.4 | 233.8 | NA | 185.4 | 278.1 | 108.8 | 117.2 | 140.2 |
| | | NA | NA | NA | | NA | NA | 4.4 | 11.0 | 6.9 |
| | 40° C. | NA | NA | NA | NA | NA | NA | NA | NA | NA |
| DSC TM2 | 5° C. | NA | 27.5 | 27.6 | NA | 29.0 | 28.9 | NA | 28.8 | 29.2 |
| | | | 0.2 | 0.4 | | 0.1 | 0.2 | | 0.2 | 0.0 |
| | 25° C. | 27.4 | 27.2 | 27.7 | 29.0 | 29.6 | 28.7 | 28.5 | 28.4 | 29.2 |
| | | 0.1 | 0.3 | 0.3 | NA | NA | 0.2 | 0.3 | 0.1 | 0.4 |
| | 40° C. | 27.4 | NA | | 28.9 | 28.0# | 27.7 | 28.3 | 27.8 | 27.7 |
| | | 0.3 | | | 0.0 | 0.3 | NA | 0.1 | 0.7 | 0.6 |
| | | | | | | | 27.4$$ | | | |
| | | | | | | | 0.1 | | | |
| DSC TM2.1 | 5° C. | NA | 33.8 | 35.0 | NA | | | NA | | |
| | | | 0.1 | 0.1 | | | | | | |
| | 25° C. | 34.6 | 34.0 | 35.5 | | | | | | |
| | | 0.2 | 0.1 | 0.6 | | | | | | |
| | 40° C. | | NA | 33.3 | | | | | | 30.9@ |
| | | | | NA | | | | | | |
| DSC TM3.1 | 5° C. | | | | NA | | | NA | | |
| | 25° C. | | | | | | | | | |
| | 40° C. | | | | 41.3 | 40.0# | 38.0# | 39.7@ | 35.0 | |
| | | | | | 0.1 | 0.2 | 0.1 | | 0.3 | |
| DSC TM3 | 5° C. | NA | 43.8 | 43.9 | NA | 46.1 | 46.1 | NA | 45.7 | 45.5 |
| | | | 0.1 | NA | | 0.2 | 0.3 | | 0.2 | 0.1 |
| | 25° C. | 43.4 | 44.0 | 44.6 | 45.8 | 46.3 | 46.3 | 45.6 | 44.5 | 44.5 |
| | | 0.0 | 0.0 | 0.6 | NA | NA | 0.3 | 0.0 | 0.2 | 0.4 |
| | 40° C. | | 46.3 | 47.9 | 47.0 | 46.0# | 44.0 | 45.5 | 44.4 | 45.2 |
| | | | 0.6 | NA | 0.5 | 0.3 | 0.1 | 0.0 | 0.3 | 0.3 |
| | | | | | | | 47.7$$ | | | |
| | | | | | | | 0.1 | | | |
| DSC TM4.2 | 5° C. | | | | NA | | | NA | | |
| | 25° C. | | | | | | | | | |
| | 40° C. | | | | | | | | | |
| DSC TM4.1 | 5° C. | | | | NA | | | NA | | |
| | 25° C. | | | | | | | | | |
| | 40° C. | | | | | | | | | |
| DSC TM4 | 5° C. | | | | NA | | | NA | | |
| | 25° C. | | | | | | | | | |
| | 40° C. | | | | | | | | | |

*The amount of light mineral oil in the formulation is adjusted based on the amount of minocycline hydrochloride and/or adapalene.
**The amount of minocycline hydrochloride in the formulation is adjusted by the potency of the minocycline hydrochloride.
$Separated into two phases. Results presented for upper phase.
$$Separated into two phases. Result for lower phase presented below the result for upper phase.
#Separated into two phases. Results presented for lower phase.
@Parameter present in only one replicate.
Formulation 190708N showed phase separation (see FIG. 13H)

### Example 12

Formulations with Different Emollients and Alternative Emollient Ratios

Formulations containing 1.2% hydrogenated castor oil (HCO), 3% minocycline HCl, and 0.3% adapalene (MCH+ADP), were prepared by a holding process with different amounts of soybean oil, coconut oil and/or isopropyl myristate or MCT oil.

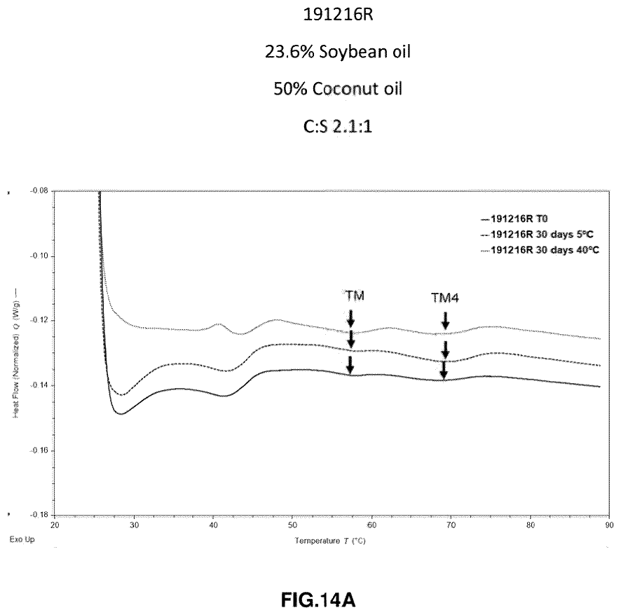

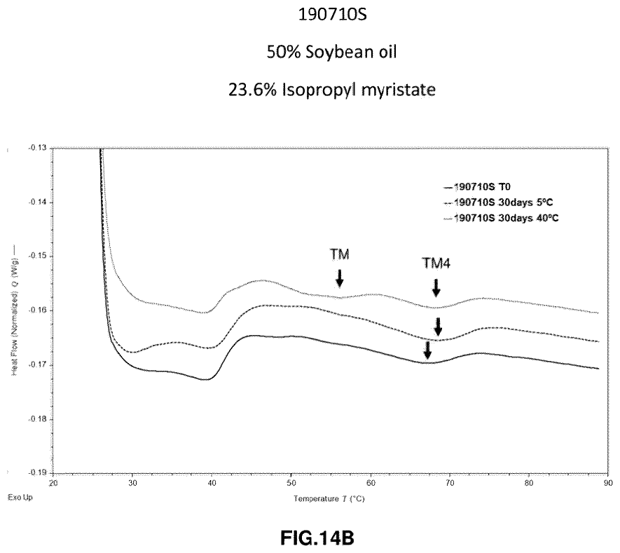

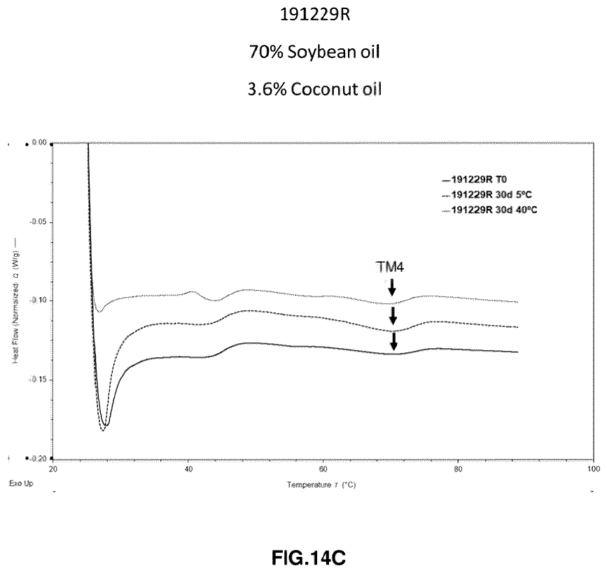

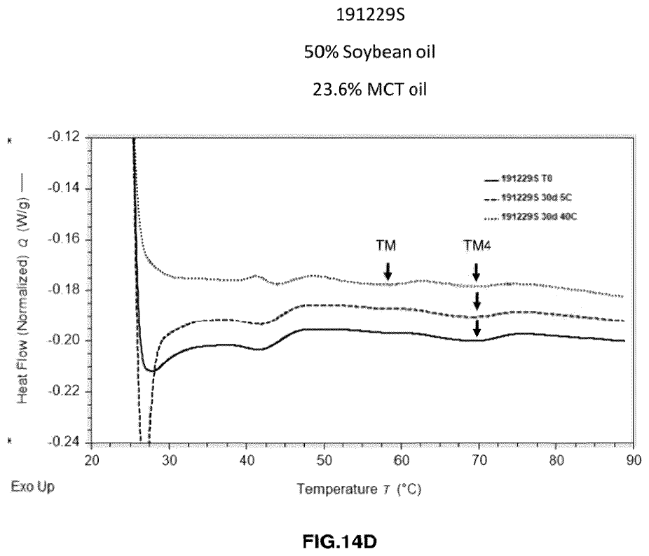

A formulation containing 23.6%/soybean oil and 50% coconut oil (191216R) showed a small increase in flow point temperature when stored at 25° C. and 40° C. For example, flow point temperature for a formulation stored at 40° C. after 15 days was 57° C. This value was higher than the flow point for formulation 190505S (that showed optimal shakability and stability). DSC analysis showed the presence of TM4 and a lower transition that were stable at all time points tested. (FIG. 14A). Formulation was shakable on day 21 at 5° C. and 25° C. and moderately shakable at 40° C. On day 30 at 40° C. formulation was not shakable.

Formulation containing isopropyl myristate instead of coconut oil (190710S) showed on day 30, an increase in flow point temperature in samples stored at 25° C. (39° C.) and 40° C. (58.5° C.) relative to formulation 190505S (36° C. and 55° C., respectively). DSC analysis showed TM4 of reduced enthalpy and a transition of lower temperature in a sample stored at 40° C. for 30 days. TM4 seemed to be fading while the enthalpy of the lower temperature transition increased (FIG. 14B). Formulation was shakable on day 15 at 5° C. and 25° C. and moderately shakable when stored for 15 days at 40° C. On day 30 at 40° C. formulation was not shakable.

A formulation containing 70% soybean oil and 3.6% coconut oil (191229R) showed normal flow point temperatures when stored at 40° C. that were similar to those of formulation 190505S. Moreover, DSC analysis showed stable TM4 peak at all conditions tested indicating stability of the formulation (FIG. 14C). Formulations stored at 5° C. and 25° C. were shakable at all timepoints tested. Formulations stored at 40° C. were moderately shakable on day 15 and non-shakable on day 30.

A formulation containing 50% soybean oil and 23.6% MCT oil (191229S) showed increased flow point temperatures for formulations stored at 40° C. on day 15 (57.2° C.) and day 30 (59° C.) relative to formulation 190505S (53.7° C. and 54.9° C., respectively). DSC analysis showed TM4 was stable but of small enthalpy at all conditions tested. A transition of lower temperature appeared in formulations stored at 40° C. (FIG. 14D). Formulations tested at 25° C. were shakable on day 0 and day 15 and moderately shakable on day 30. Formulations stored at 40° C. were non-shakable on day 15 and day 30.

Formulations without coconut oil (190710S and 191229S) showed either changes in TM4 peak or high flow points that may indicate formulation instability and potential tendency to form blockage of the formulation. Thus, these results indicated the importance of coconut oil in the formulation.

TABLE 21a

| | | 3.9 | | | 8.2 | | | 8.3 | | | 9.1 | | | 9.2 | | |
|---|---|---|---|---|---|---|---|---|---|---|---|---|---|---|---|---|
| Component | | 190505S | | | 191216R | | | 191229R | | | 190710S | | | 191229S | | |
| Soybean oil | | 50 | | | 23.6 | | | 70 | | | 50 | | | 50 | | |
| coconut oil | | 23.6 | | | 50 | | | 3.6 | | | 0 | | | 0 | | |
| Isopropyl | | 0 | | | 0 | | | 0 | | | 23.6 | | | 0 | | |
| myristate | | | | | | | | | | | | | | | | |
| MCT oil | | 0 | | | 0 | | | 0 | | | 0 | | | 23.6 | | |
| Light mineral | | 3.3 | | | 3.3 | | | 3.3 | | | 3.3 | | | 3.3 | | |
| oil* | | | | | | | | | | | | | | | | |
| Stearic acid | | 3 | | | 3 | | | 3 | | | 3 | | | 3 | | |
| Docosanol | | 1.1 | | | 1.1 | | | 1.1 | | | 1.1 | | | 1.1 | | |
| Hydrogenated | | 1.2 | | | 1.2 | | | 1.2 | | | 1.2 | | | 1.2 | | |
| castor oil | | | | | | | | | | | | | | | | |
| White wax | | 2 | | | 2 | | | 2 | | | 2 | | | 2 | | |
| (beeswax) | | | | | | | | | | | | | | | | |
| Stearyl alcohol | | 1.5 | | | 1.5 | | | 1.5 | | | 1.5 | | | 1.5 | | |
| Cetostearyl | | 3.5 | | | 3.5 | | | 3.5 | | | 3.5 | | | 3.5 | | |
| alcohol | | | | | | | | | | | | | | | | |
| Myristyl alcohol | | 2.5 | | | 2.5 | | | 2.5 | | | 2.5 | | | 2.5 | | |
| Cyclomethicone | | 5 | | | 5 | | | 5 | | | 5 | | | 5 | | |
| Minocycline | | 3 | | | 3 | | | 3 | | | 3 | | | 3 | | |
| HCl** | | | | | | | | | | | | | | | | |
| Adapalene | | 0.3 | | | 0.3 | | | 0.3 | | | 0.3 | | | 0.3 | | |
| TOTAL | | 100 | | | 100 | | | 100 | | | 100 | | | 100 | | |
| Ratio Coconut: | | 0.5:1 | | | 2.1:1 | | | 0.05:1 | | | — | | | — | | |
| Soybean oil | | | | | | | | | | | | | | | | |
| | Tem- | | | | | | | | | | | | | | | |
| | per- | | | | | | | | | | | | | | | |
| | ature/ | | | | | | | | | | | | | | | |
| Test | Time | T0 | T15 | T30 | T0 | T15 | T30 | T0 | T15 | T30 | T0 | T15 | T30 | T0 | T15 | T30 |
| Shakability | 5° C. | S | S | S | N/A | S# | S | N/A | S | S | N/A | S | S | N/A | S | S |
| | 25° C. | S | S | S | N/A | S# | S | N/A | S | S | N/A | S | S | N/A | S | M |
| | 40° C. | S | M | M | N/A | M# | N | N/A | M | N | N/A | M | N | N/A | N | N |
| Rheology | 5° C. | NA | 122.1 | 128.9 | NA | 51 | 66 | NA | 180 | 880 | NA | 46 | 62 | NA | 1111 | 1307 |
| G' | | | 11.1 | 27.3 | | 2.1 | 14.8 | | 0.7 | 178.2 | | 21.9 | 16.3 | | 30.4 | 71.4 |
| Pa | 25° C. | 58.9 | 346.4 | 434.2 | 67 | 169 | 280 | 91 | 163 | 341 | 48 | 222 | 555 | 76 | 238 | 1128 |
| | | 1.4 | 29.5 | 10.9 | 8.5 | 5.7 | 3.5 | 11.3 | 12.0 | 4.9 | 1.8 | 30.4 | 72.8 | 9.5 | 25.8 | 43.8 |
| | 40° C. | 12372.5 | 7884.6 | 5933.6 | 203910 | 5376 | 6507 | 16529 | 5347 | 5242 | 3073 | 7271 | 5702 | 7046 | 5731 | 7213 |
| | | 482.2 | 1891.3 | 584.7 | 58 | 974.4 | 213.5 | 585.5 | 587.6 | 2143.9 | NA | 2935 | 1.4 | 1169.2 | 557.9 | 723.7 |
| Rheology | 5° C. | NA | 26.4 | 26.7 | NA | 25.7 | 27.1 | NA | 26.7 | 27.2 | NA | 25.7 | 25.6 | NA | 27.1 | 26.9 |
| Flow | | | 0.8 | 1.1 | | 0.9 | 0.0 | | 0.0 | 0.1 | | 0.4 | 0.2 | | 0.0 | 0.0 |
| Point, | 25° C. | 24.9 | 34.6 | 36.3 | 26.4 | 34.3 | 35.9 | 27.6 | 34.0 | 38.0 | 25.8 | 32.7 | 39.3 | 26.7 | 33.9 | 37.0 |
| ° C. | | 0.5 | 0.5 | 0.2 | NA | 0.9 | 0.2 | 1.9 | 0.3 | 0.0 | 0.1 | 0.6 | 1.5 | 0.3 | 0.3 | 0.1 |
| | 40° C. | 32.2 | 53.65 | 54.85 | 31.3 | 56.8 | 58.4 | 30.6 | 51.1 | 55.0 | 34.5 | 56.8 | 58.5 | 31.9 | 57.2 | 59.0 |
| | | 0.0 | 0.5 | 1.3 | 0.2 | 0.2 | 0.1 | 0.0 | 0.1 | 0.5 | NA | 0.5 | 0.1 | 0.4 | 0.3 | 0.0 |
| Rheology | 5° C. | NA | 244.5β | 291.6 β | NA | | 256.4 | NA | 331.4 | 420.0 | NA | 212.9 | 205.8 | NA | 399.0 | 416.2 |
| Viscosity, | | | | | | | NA | | NA | NA | | | | | | |
| cP | 25° C. | 251.1 β | File | 368.9 β | 252.7 | | 305.5 | 237.4 | 309.4 | 343.8 | 154.6 | 257.2 | 320.5 | 234.9 | 385.0 | 583.7 |
| | | | not | | | | NA | NA | NA | NA | NA | NA | NA | NA | NA | NA |
| | | | found | | | | | | | | | | | | | |
| | 40° C. | NA | NA | NA | NA | NA | NA | NA | NA | NA | NA | NA | NA | NA | NA | NA |
| DSC TM2 | 5° C. | NA | 27.1 | 26.7 | NA | 28.0 | 28.5 | NA | 27.9 | 27.5 | | | | NA | 26.9 | 26.9 |
| | | | 0.1 | 0.1 | | 0.3 | 0.1 | | 0.4 | 0.1 | | | | | 0.1 | 0.0 |
| | 25° C. | 26.9 | 27.1 | 27.0 | 28.3 | 28.3 | 28.5 | 27.8 | 27.9 | 27.6 | | | | 27.7 | 28.1 | 27.6 |
| | | 0.1 | 0.1 | 0.1 | 0.0 | 0.0 | 0.1 | 0.1 | 0.1 | 0.1 | | | | 0.2 | 0.1 | 0.0 |
| | 40° C. | 26.5 | | | | | | 27.3 | 27.2 | 26.9 | | | | 26.5 | | |
| | | 0.0 | | | | | | 0.1 | 0.0 | 0.1 | | | | 0.0 | | |
| DSC | 5° C. | NA | | | | | | | | | | 30.4 | 30.2 | | | |
| TM2.1 | | | | | | | | | | | | 0.1 | 0.1 | | | |
| | 25° C. | | | | | | 35.6 | 37.3 | | | | | | | 35.1 | |
| | | | | | | | 0.1 | 0.6 | | | | | | | 0.4 | |
| | 40° C. | 31.9 | 29.2 | 30.3 | | | | | | | | | | 31.6 | | |
| | | 0.1 | 2.0 | 0.3 | | | | | | | | | | 0.3 | | |
| DSC | 5° C. | NA | | | | | | | | | | | | | | |
| TM3.1 | 25° C. | | | | | | | | | | | | | | | |
| | 40° C. | 39.9 | 38.3 | 38.8 | | 39.3 | 39.0 | 40.5 | | 39.1 | | 39.6 | | 40.4 | 39.9 | 40.0 |
| | | 0.3 | 0.1 | 0.3 | | 0.1 | 0.2 | 0.1 | | 0.3 | | 0.2 | | 0.1 | 0.1 | 0.0 |
| DSC | 5° C. | NA | 42.4 | 42.1 | NA | 42.2 | 42.4 | NA | 44.4 | 44.0 | NA | 41.2 | 41.0 | NA | 42.8 | 42.3 |
| TM3 | | | 0.1 | 0.1 | | 0.1 | 0.1 | | 0.2 | 0.2 | | 0.2 | 0.4 | | 0.3 | 0.1 |
| | 25° C. | 42.1 | 42.3 | 41.9 | 42.1 | 42.9 | 42.7 | 44.1 | 44.0 | 43.8 | 40.7 | 41.2 | 42.2 | 42.2 | 43.2 | 43.1 |
| | | 0.2 | 0.7 | 0.2 | 1.2 | 0.1 | 0.57 | 0.3 | 0.1 | 0.4 | 0.2 | 0.1 | 0.6 | 0.1 | 0.0 | 0.1 |
| | 40° C. | 43.5 | 43.8 | 44.1 | NA | 42.6 | 43.2 | 43.8 | 43.8 | 43.9 | 40.5 | 42.6 | 40.1 | 43.1 | 43.4 | 43.5 |
| | | 0.1 | 0.1 | 0.2 | | 0.1 | 0.1 | 0.1 | 0.0 | 0.0 | 0.1 | 0.2 | 0.4 | 0.1 | 0.1 | 0.1 |
| DSC | 5° C. | NA | | | NA | 57.5 | 58.1 | | | | | | | | | |
| TM4.2 | | | | | | 0.1 | 0.2 | | | | | | | | | |
| | 25° C. | | | | 58.0 | 58.5 | 58.6 | | | | | | | | | |
| | | | | | 0.1 | 0.1 | 0.4 | | | | | | | | | |
| | 40° C. | | 53.7 | 56.1 | NA | 57.5 | 57.7 | | | | | | 56.1 | 57.7 | 56.4 | 58.1 |
| | | | 0.1 | 0.5 | | 0.3 | 0.5 | | | | | | 0.1 | 0.1 | 0.9 | 0.3 |
| DSC | 5° C. | NA | | | | | | | | | | | | | | |
| TM4.1 | 25° C. | | | | | | | | | | | | | | | |
| | 40° C. | | | | | | 66.8 | | | | | | | | | |
| | | | | | | | 0.49 | | | | | | | | | |
| DSC | 5° C. | | | | NA | 69.3 | 69.1 | NA | 70.3 | 70.4 | NA | 68.1 | 68.6 | NA | 68.9 | 69.7 |
| TM4 | | | | | | 0.8 | 0.4 | | 0.1 | 0.1 | | 0.2 | 0.0 | | 0.6 | 0.7 |
| | 25° C. | | | | 68.5 | 69.4 | 68.7 | 70.3 | 70.4 | 70.5 | 67.5 | 68.7 | 68.1 | 69.4 | 69.1 | 69.5 |
| | | | | | 0.4 | 0.5 | 0.1 | 0.2 | 0.1 | 0.3 | 0.0 | 0.5 | 0.3 | 0.4 | 0.6 | 0.6 |
| | 40° C. | | | | | 68.3 | 70.2 | 70.2 | 69.8 | 69.6 | 67.4 | 68.1 | 68.3 | 69.5 | 69.7 | 68.3 |
| | | | | | | 0.0 | 0.2 | 0.1 | 0.4 | 0.3 | 0.9 | 0.4 | 0.1 | 0.0 | 0.3 | 0.2 |

#Shakability was evaluated on day 21.
*The amount of light mineral oil in the formulation is adjusted based on the amount of minocycline hydrochloride and/or adapalene.
**The amount of minocycline hydrochloride in the formulation is adjusted by the potency of the minocycline hydrochloride
βOnly one replicate was performed

TABLE 21b

| | Placebo (70% |
|---|---|
| | soybean oil; |
| | 3.6% coconut |
| | oil) |
| Component | |
| Soybean oil | 70 |
| coconut oil | 3.6 |
| Isopropyl myristate | 0 |
| MCT oil | 0 |
| Light mineral oil* | 6.6 |
| Stearic acid | 3 |
| Docosanol | 1.1 |
| Hydrogenated castor oill | 1.2 |
| White wax (beeswax) | 2 |
| Stearyl alcohol | 1.5 |
| Cetostearyl alcohol | 3.5 |
| Myristyl alcohol | 2.5 |
| Cyclomethicone | 5 |
| Minocycline HCl** | 0 |
| Adapalene | 0 |
| TOTAL | 100 |
| Ratio Coconut:Soybean oil | 0.05:1 |

| | Temperature/ | | | |
|---|---|---|---|---|
| Test | Time | T0 | T15 | T30 |
| Shakability | 5° C. | | | |
| | 25° C. | | | |
| | 40° C. | | | |
| Rheology G′ | 5° C. | NA | 228.6 | 286.4 |
| Pa | | | 22.1 | 59.3 |
| | 25° C. | 111.0 | 241.6 | 214.3 |
| | | 7.6 | 5.8 | 54.3 |
| | 40° C. | 18072.2 | 16355.1 | 5210.5 |
| | | 2277.3 | 2816.1 | 811.9 |
| Rheology | 5° C. | NA | 27.4 | 28.0 |
| Flow Point, | | | 0.2 | 0.9 |
| ° C. | 25° C. | 28.7 | 32.9 | 34.0 |
| | | 0.4 | 1.0 | 0.6 |
| | 40° C. | 32.9 | 57.2 | 57.9 |
| | | 0.3 | 0.5 | 1.2 |
| Rheology | 5° C. | NA | 370.4 | 389.7 |
| Viscosity, cP | | | 17.1 | 4.3 |
| | 25° C. | 239.8 | 286.0 | 348.7 |
| | | 23.6 | 15.0 | 3.5 |
| | 40° C. | NA | NA | NA |
| DSC TM2 | 5° C. | NA | 27.5 | 28.1 |
| | | | 0.1 | 0.0 |
| | 25° C. | 27.2 | 27.5 | |
| | | 0.7 | 0.2 | |
| | 40° C. | 27.6 | 27.0 | 27.5 |
| | | 0.4 | 0.4 | 0.1 |
| DSC TM2.1 | 5° C. | NA | 30.5 | |
| | | | 0.1 | |
| | 25° C. | 30.8 | 30.6 | 29.8 |
| | | 0.9 | 0.4 | 0.1 |
| | 40° C. | | 34.5 | 33.2 |
| | | | 0.2 | 0.9 |
| DSC TM3.1 | 5° C. | NA | 37.2 | |
| | | | @ | |
| | 25° C. | 37.6 | | |
| | | @ | | |
| | 40° C. | 39.6 | | |
| | | 0.5 | | |
| DSC TM3 | 5° C. | NA | 43.7 | 43.7 |
| | | | 0.1 | 0.1 |
| | 25° C. | 43.4 | 44.2 | 43.8 |
| | | 0.3 | 0.5 | 0.0 |
| | 40° C. | 44.1 | 44.0 | 44.1 |
| | | 0.3 | 0.3 | 0.3 |
| DSC TM4.2 | 5° C. | NA | | |
| | 25° C. | | | |
| | 40° C. | | | |
| DSC TM4.1 | 5° C. | NA | 57.9 | 57.9 |
| | | | 0.0 | 0.1 |
| | 25° C. | 57.4 | 57.9 | 57.8 |
| | | 0.1 | 1.1 | 0.1 |
| | 40° C. | 57.5 | 56.6 | 59.9 |
| | | 0.4 | 0.7 | 0.0 |
| DSC TM4 | 5° C. | NA | 71.1 | 71.7 |
| | | | 0.4 | 0.3 |
| | 25° C. | 71.1 | 71.2 | 71.7 |
| | | 0.0 | 0.3 | 0.4 |
| | 40° C. | 71.4 | 71.3 | 71.2 |
| | | 0.5 | 0.1 | 0.4 |

@Parameter present in only one replicate.

### Example 13

### Oil Studies

Mixtures of different oils with hydrogenated castor oil (HCO) with APIs (MCH and ADP) were prepared in a holding process and analyzed by DSC and microscopy as described below and shown in Tables 22A-B.

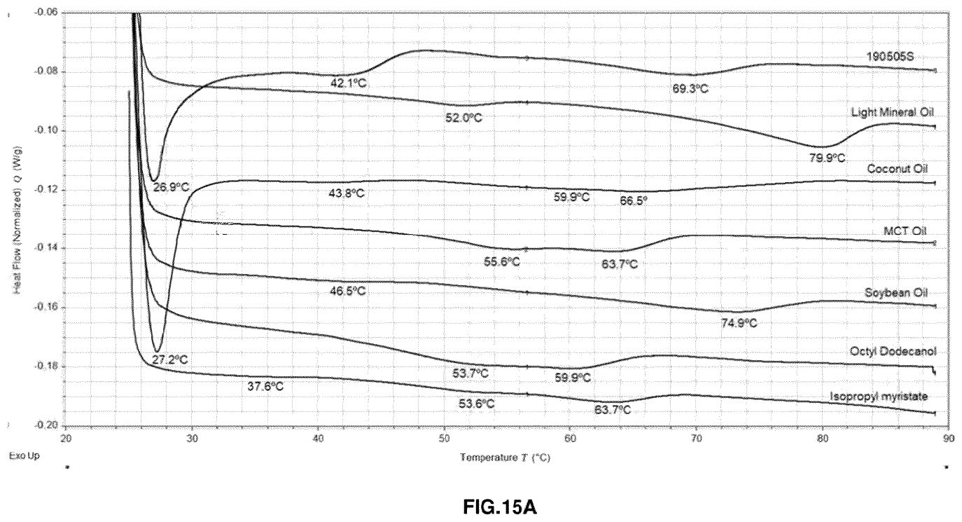

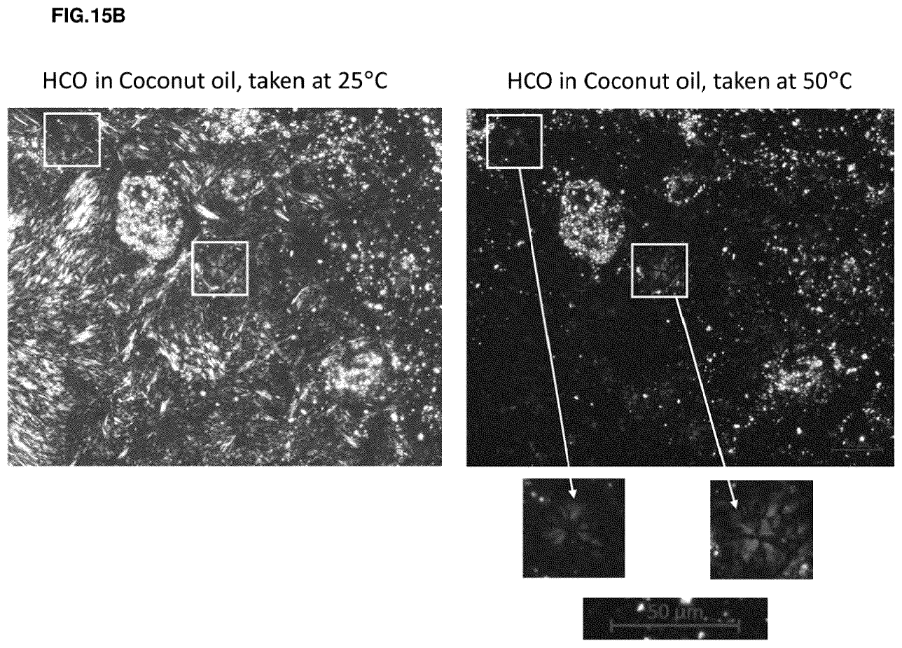

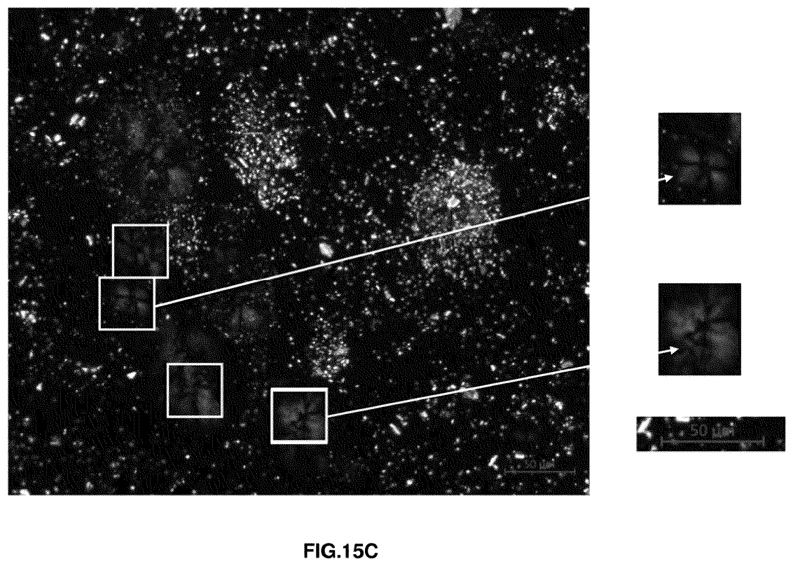

As can be seen in FIG. 15A, only mixtures of HCO in light mineral oil, soybean oil and coconut oil presented TM4 peak. Mixtures of HCO in soybean oil and light mineral oil showed TM4 peak at 74.9° C. and 79.9° C., respectively. Mixture of HCO in coconut oil showed TM4 at 66.5° C.

Microscopic analysis of mixtures of HCO in coconut oil and octyldodecanol showed spherulites that are typical for formulations prepared in a continuous heating-cooling process (FIG. 15B, 15C). It may be, however, that this phenomenon relates to the preparation of these mixtures in the DSC that differ from preparing the full formulation (as DSC is not a closed system with purging with nitrogen and mixing under vacuum).

Similar studies may be conducted evaluating additional mixtures with APIs. A mixture of di-isopropyl adipate with hydrogenated castor oil (HCO) with APIs (MCH and ADP) is prepared in a holding process and analyzed by DSC and microscopy as described below and shown in Table 22C.

Samples of HCO in soybean oil without APIs are prepared in a holding process. Tmh crystals are concentrated by centrifugation according to the methodology described hereinabove. Precipitants which contain the concentrated Tmh crystals are collected and analyzed by DSC and microscopy as described and shown in Table 22D.

Samples of cyclomethicone and/or elastomer with APIs (MCH and ADP) and without APIs are prepared in a holding process. Tmh crystals are concentrated by centrifugation according to the methodology described hereinabove. Precipitants which contain the concentrated Tmh crystals are collected and analyzed by microscopy as described and shown in Table 22E. Due to the volatile nature of cyclomethicone and elastomer, and evaporation of the sample, they are not measured by DSC.

TABLE 22A

| Component | | | |
|---|---|---|---|
| Soybean oil | 95.5 | | |
| Coconut oil | | 95.5 | |
| Isopropyl myristate | | | 95.5 |
| Hydrogenated castor oil | 1.2 | 1.2 | 1.2 |
| Minocycline HCl | 3.0 | 3.0 | 3.0 |
| Adapalene | 0.3 | 0.3 | 0.3 |
| Total | 100 | 100 | 100 |

| | Temperature/ | | | |
|---|---|---|---|---|
| Test | Time | T0 | T0 | T0 |
| DSC TM2 | 25° C. | | 27.2 | |
| DSC TM2.1 | 25° C. | | | |
| DSC TM3.1 | 25° C. | | | 37.6 |
| DSC TM3 | 25° C. | 46.5 | 43.8 | |
| DSC TM4.2 | 25° C. | | | 53.6 |
| DSC TM4.1 | 25° C. | | 59.9 | 63.7 |
| DSC TM4 | 25° C. | 74.9 | 66.5 | |

TABLE 22B

| Component | | | |
|---|---|---|---|
| Light mineral oil | 95.5 | | |
| MCT oil | | 95.5 | |
| Octyldodecanol | | | 95.5 |
| Hydrogenated castor oil | 1.2 | 1.2 | 1.2 |
| MCH | 3 | 3 | 3 |
| ADP | 0.3 | 0.3 | 0.3 |
| Total | 100 | 100 | 100 |

| | Temperature/ | | | |
|---|---|---|---|---|
| Test | Time | T0 | T0 | T0 |
| DSC TM4.2 | 25° C. | 52.0 | 55.6 | 53.7 |
| DSC TM4.1 | 25° C. | | 63.7 | 59.9 |
| DSC TM4 | 25° C. | 79.9 | | 78.6 |

TABLE 22C

| Component | |
|---|---|
| Di-isopropyl adipate | 95.5 |
| Hydrogenated castor oil | 1.2 |
| MCH | 3 |
| ADP | 0.3 |
| Total | 100 |

TABLE 22D

| Component | |
|---|---|
| Soybean oil | 98.8 |
| Hydrogenated castor oil | 1.2 |
| MCH | — |
| ADP | — |
| Total | 100 |

TABLE 226

| Component | | |
|---|---|---|
| Elastomer* | 95.5 | |
| Cyclomethicone* | | 95.5 |
| Hydrogenated castor oil | 1.2 | 1.2 |
| Minocycline HCl | 3.0 | 3.0 |
| Adapalene | 0.3 | 0.3 |
| Total | 100 | 100 |

| | Temperature/ | | |
|---|---|---|---|
| Test | Time | T0 | T0 |
| DSC TM2 | 25° C. | * | * |
| DSC TM2.1 | 25° C. | * | * |
| DSC TM3.1 | 25° C. | * | * |
| DSC TM3 | 25° C. | * | * |
| DSC TM4.2 | 25° C. | * | * |
| DSC TM4.1 | 25° C. | * | * |
| DSC TM4 | 25° C. | * | * |

*Cyclomethicone and Elastomer could not be tested due to their volatile nature and evaporation of the sample.

### Example 14. A Formulation Prepared in a Process Including Two Holding Steps

A formulation containing 1.2% hydrogenated castor oil (HCO), 3% minocycline HCl, and 0.3% adapalene (MCH+ADP), was prepared by a holding process with two holding steps at 54° C. for 3 hours and at 40° C. for 3 hours (200105R).

Formulations stored at 40° C. for 15 days and 30 days showed a small increase in flow point temperature compared to formulation 190505S (56.4° C. and 58° C. vs. 53.7° C. and 54.9° C., respectively). Formulations stored at 5° C. and 25° C. in all timepoints tested were shakable. Formulations stored at 40° C. for 15 days and 30 days were non-shakable.

TABLE 23

| Component | 200105R |
|---|---|
| Soybean oil | 50 |
| coconut oil | 23.6 |
| isopropyl myristate | 0 |
| MCT oil | 0 |
| Light mineral oil* | 3.3 |
| Stearic acid | 3 |
| Docosanol | 1.1 |
| Hydrogenated castor oil | 1.2 |
| White wax (beeswax) | 2 |
| Stearyl alcohol | 1.5 |
| Cetostearyl alcohol | 3.5 |
| Myristyl alcohol | 2.5 |
| Cyclomethicone | 5 |
| Minocycline HCl** | 3 |
| Adapalene | 0.3 |
| TOTAL | 100 |

| | Temperature/ | | | |
|---|---|---|---|---|
| Test | Time | T0 | T15 | T30 |
| Shakability | 5° C. | S | S | S |
| | 25° C. | S | S | S |
| | 40° C. | S | N | N |
| Rheology G′ | 5° C. | NA | 51 | 121 |
| Pa | | | 8.5 | 11.3 |
| | 25° C. | 45 | 120 | 266 |
| | | 1.4 | 6.4 | 24.0 |
| | 40° C. | 7794 | 6251 | 6063 |
| | | 886.0 | 1562.0 | 620.8 |
| Rheology | 5° C. | NA | 27.1 | 26.1 |
| Flow Point, | | | 0.1 | 0.4 |
| ° C. | 25° C. | 25.7 | 31.1 | 35.0 |
| | | 0.6 | 1.1 | 0.2 |
| | 40° C. | 31.6 | 56.4 | 58.0 |
| | | 0.1 | 1.0 | 0.3 |
| Rheology | 5° C. | NA | 2411 | 2684 |
| Viscosity, | 25° C. | 2174 | 2711 | 2958 |
| cP | 40° C. | NA | NA | NA |
| DSC TM2 | 5° C. | | 27.4 | 27.3 |
| | | | 0.1 | 0.1 |
| | 25° C. | 28.1 | 27.6 | 26.9 |
| | | 0.9 | 0.4 | 0.1 |
| | 40° C. | 27.3 | 27.0 | |
| | | 0.1 | 0.1 | |
| DSC TM2.1 | 5° C. | | | |
| | 25° C. | | 36.1 | 35.7 |
| | | | 0.1 | 0.3 |
| | 40° C. | 32.1 | | |
| | | 0.3 | | |
| DSC TM3.1 | 5° C. | | | |
| | 25° C. | | | |
| | 40° C. | 40.9 | 39.5 | 40.2 |
| | | 0.1 | 0.0 | 0.1 |
| DSC TM3 | 5° C. | | 43.2 | 44.1 |
| | | | 0.3 | 0.4 |
| | 25° C. | 44.0 | 43.8 | 43.2 |
| | | 0.7 | 0.1 | 0.1 |
| | 40° C. | 44.1 | 43.9 | 43.8 |
| | | 0.1 | 0.1 | 0.0 |
| DSC TM4.2 | 5° C. | | 58.3 | 58.8 |
| | | | 0.0 | 0.78 |
| | 25° C. | | 59.0 | 58.9 |
| | | | 0.5 | 0.4 |
| | 40° C. | 59.1 | 58.6 | 58.4 |
| | | 0.1 | 0.1 | 0.1 |
| DSC TM4.1 | 5° C. | | | |
| | 25° C. | | | |
| | 40° C. | | | |
| DSC TM4 | 5° C. | | 70.1 | 70.3 |
| | | | 0.1 | 1.2 |
| | 25° C. | 70.2 | 70.5 | 69.9 |
| | | 0.4 | 0.1 | 0.1 |
| | 40° C. | 70.8 | 69.7 | 69.1 |
| | | 0.4 | 0.5 | 0.8 |

*The blanks within the table indicate that no peak was observed.

### Example 15. A Formulation Prepared in a Process Including a Longer Holding step

A formulation containing 1.2% hydrogenated castor oil (HCO), 3% minocycline HCl, and 0.3% adapalene (MCH+ADP), was prepared by a holding process with a holding step at 54° C. that lasted for about 16 hours (See Table 24 re 200107D).

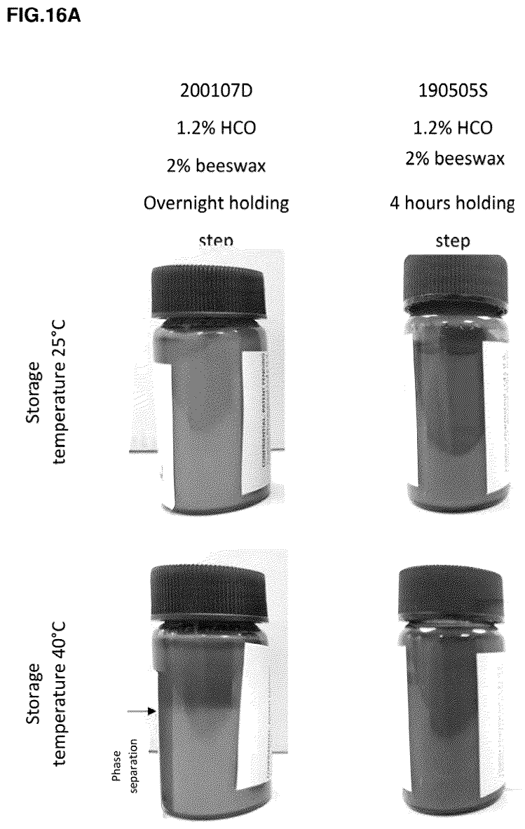

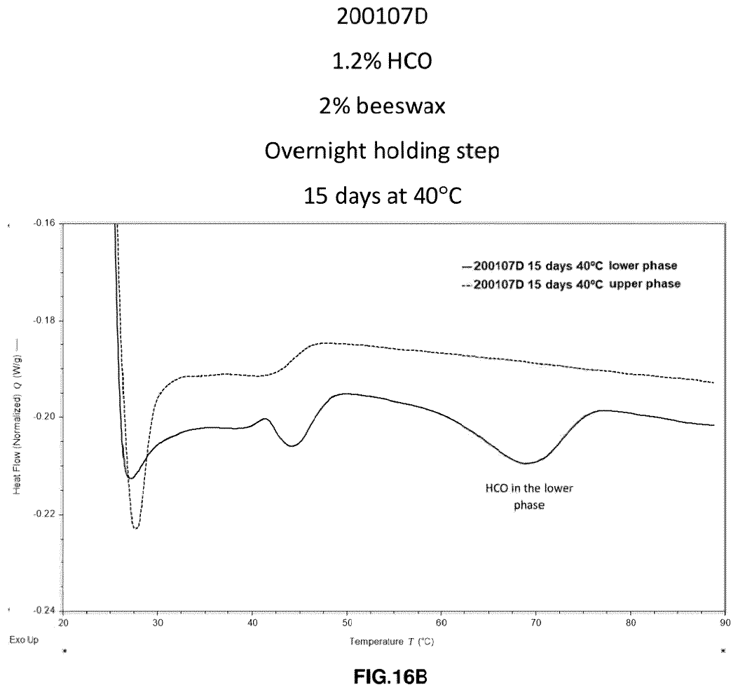

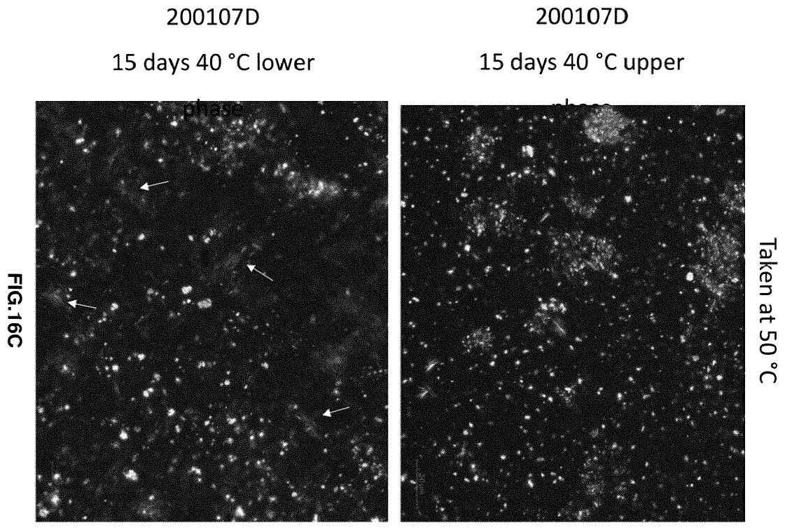

Formulation prepared in the longer holding process (about 16 hours) resulted in phase separation and sedimentation of active agent(s) when stored at 40° C. (FIG. 16A). DSC and microscopy analysis showed the presence of HCO in the lower phase of the formulation. (FIG. 16B, C)

TABLE 24

| Component | 200107D |
|---|---|
| Soybean oil | 50 |
| coconut oil | 23.6 |
| Isopropyl myristate | 0 |
| MCT oil | 0 |
| Light mineral oil* | 3.3 |
| Stearic acid | 3 |
| Docosanol | 1.1 |
| Hydrogenated castor oil | 1.2 |
| White wax (beeswax) | 2 |
| Stearyl alcohol | 1.5 |
| Cetostearyl alcohol | 3.5 |
| Myristyl alcohol | |
| Cyclomethicone | 5 |
| Minocycline HCl** | 3 |
| Adapalene | 0.3 |
| TOTAL | 100 |

| | Temperature/ | | | | |
|---|---|---|---|---|---|
| Test | Time | | T0 | T15 | T30 |
| Shakability | 5° | C. | S | S | S |
| | 25° | C. | S | S | S |
| | 40° | C. | S | S | M |
| Rheology G′ | 5° | C. | NA | 35 | 76.5 |
| Pa | | | | 5.7 | 14.8 |
| | 25° | C. | 31 | 205 | 231 |
| | | | 7.8 | 24.8 | 49.5 |
| | 40° | C. | 13892 | 2387 | 711 |
| | | | 3819.1 | NA | NA |
| | | | | 5676 | 4643 |
| | | | | 784.2# | 1185# |
| Rheology | 5° | C. | NA | 26.4 | 27.4 |
| Flow Point, ° C. | | | | 0.0 | 1.1 |
| | 25° | C. | 24.2 | 33.9 | 33.3 |
| | | | 1.8 | 0.1 | 0.6 |
| | 40° | C. | 32.3 | 41.5 | 31.0 |
| | | | 0.5 | 2.3$ | NA |
| | | | | | 47.6 |
| | | | | | 0.2# |
| Rheology | 5° | C. | NA | 235.0 | 228.8 |
| Viscosity, cP | 25° | C. | 233.2 | 311.4 | 350.2 |
| | 40° | C. | NA | NA | NA |
| DSC TM2 | 5° | C. | | 28.5 | 27.1 |
| | | | | 0.3 | 0.1 |
| | 25° | C. | 27.6 | 27.2 | 27.3 |
| | | | 0.4 | 0.1 | 0.1 |
| | 40° | C. | 27.0 | 27.8 | 27.9 |
| | | | 0.3 | 0.28 | 0.0$$ |
| | | | | 27.2 | |
| | | | | 0.07# | |
| DSC TM2.1 | 5° | C. | | | |
| | 25° | C. | | | |
| | 40° | C. | | | |
| DSC TM3.1 | 5° | C. | | | |
| | 25° | C. | | | |
| | 40° | C. | 40.4 | 39.8 | 39.3 |
| | | | 0.1 | 0.1$ | 0.1$ |
| DSC TM3 | 5° | C. | | 43.7 | 43.6 |
| | | | | 0.4 | 0.2 |
| | 25° | C. | 44.0 | 42.8 | 43.1 |
| | | | 0.0 | 0.2 | 0.2 |
| | 40° | C. | 43.9 | 43.4 | 42.8 |
| | | | 0.1 | 0.0 | 0.57 |
| | | | | 44.3 | 44.4 |
| | | | | 0.4# | 0.1# |
| DSC TM4.2 | 5° | C. | | | |
| | 25° | C. | | | |
| | 40° | C. | | | |
| DSC TM4.1 | | | | | |
| | 25° | C. | | | |
| | 40° | C. | | | |
| DSC TM4 | 5° | C. | | 69.5 | 68.8 |
| | | | | 0.2 | 0.1 |
| | 25° | C. | 68.8 | 68.6 | 69.7 |
| | | | 0.5 | 0.6 | 0.1 |
| | 40° | C. | 68.6 | 69.0 | 69.2 |
| | | | 0.4 | 0.2$ | 0.0$ |

#Separated into two phases. Result for lower phase presented below the result for upper phase.
$Separated into two phases. Result presented for lower phase.
$$Separated into two phases. Result presented for upper phase.
*The blanks within the table indicate that no peak was observed.

### Example 16. Foam Quality Properties for Formulations with Alternative Waxes and Wax Ratios, Alternative Emollients and Emollient Ratios, and Alternative Process of Manufacturing

Foam quality was tested for formulations in Examples 11-12, 14-15. Measurement were performed at different time points as described in the tables below. Foams were of excellent or good qualities.

TABLE 25

| Formu- | | | | | | |
|---|---|---|---|---|---|---|
| lation # | | 191113S | 191119S | 191216R | 191229R | 191229S |
| Measure | | 3M | 3M | 2M | 2M | 2M |
| ment | | | | | | |
| time | | | | | | |
| 5° C. | Foam | E | E | E | E | E |
| | quality | | | | | |
| | Collapse | >180 | >180 | 165 | >180 | >180 |
| | time | | | | | |
| | Time to | >180 | 180 | 90 | 150 | 120 |
| | FG | | | | | |
| 25° C. | Foam | E | E | E | E | E |
| | quality | | | | | |
| | Collapse | >180 | >180 | 160 | >180 | >180 |
| | time | | | | | |
| | Time to | 180 | 150 | 120 | 150 | 150 |
| | FG | | | | | |

TABLE 26

Formulation #

| | | |
|---|---|---|
| Measurement | 200105R | 200107D |
| time | 1.5 M | 1 M |

| | | | | |
|---|---|---|---|---|
| 5° | C. | Foam quality | E | E |
| | | Collapse time | >180 | >180 |
| | | Time to FG | 120 | >180 |
| 25° | C. | Foam quality | E | E |
| | | Collapse time | >180 | >180 |
| | | Time to FG | 90 | >180 |

### Example 17. Hydrogen Bonds

In order to obtain information of the hydrogen bonds formed by the HCO contained in the formulations described herein, infrared spectra of the following formulations were obtained: Formulations containing 1.2% or 2% HCO prepared by a continuous heating-cooling process and a holding process, Oils (soybean oil, coconut oil, mineral oil and cyclomethicone in the same proportion as in the full formulation), 1.2% HCO in oils, and neat HCO.

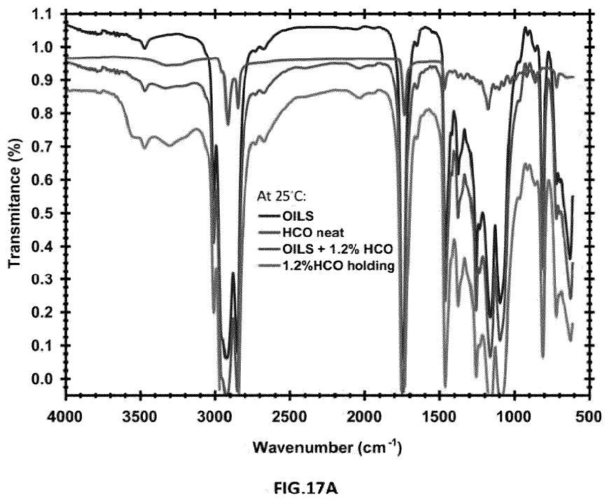

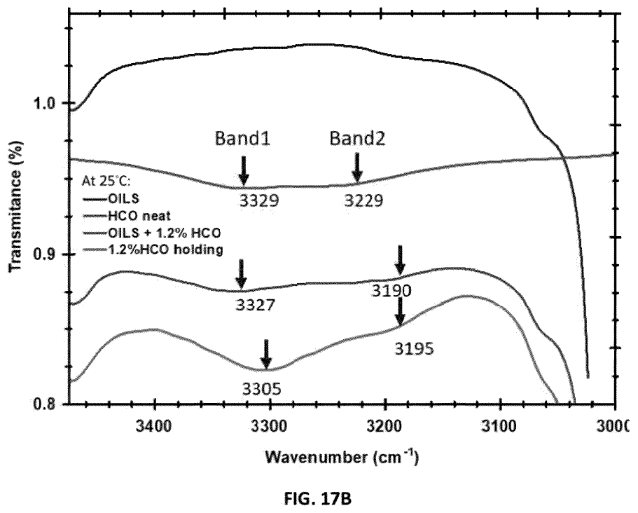

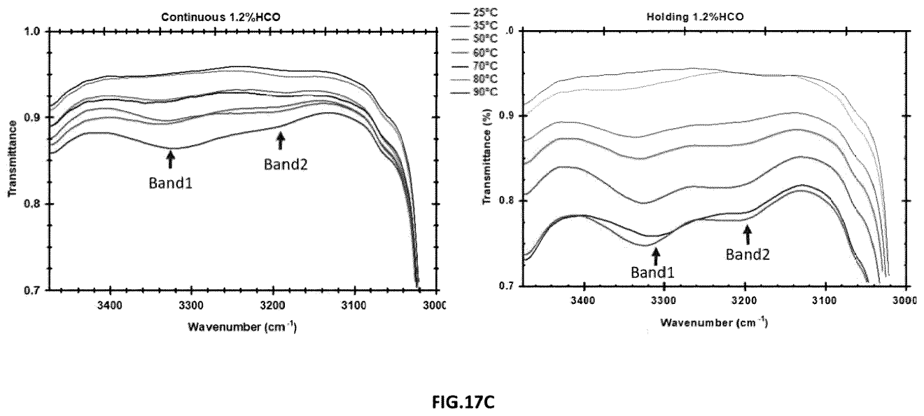

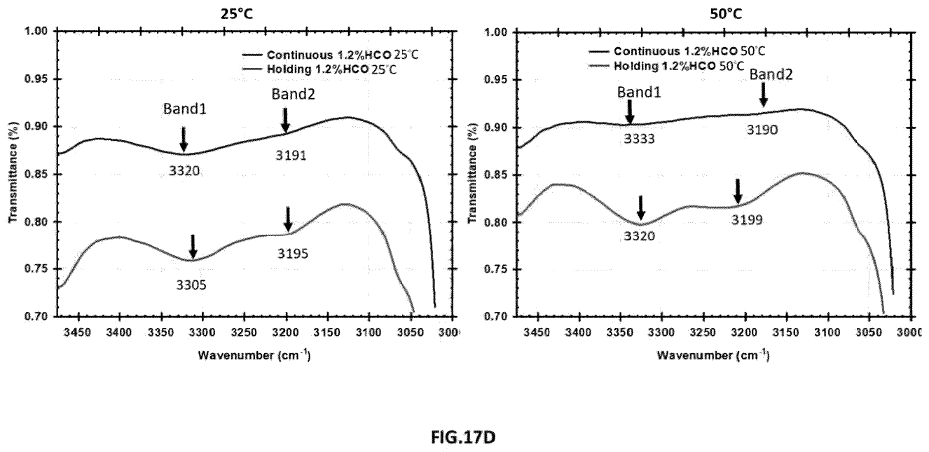

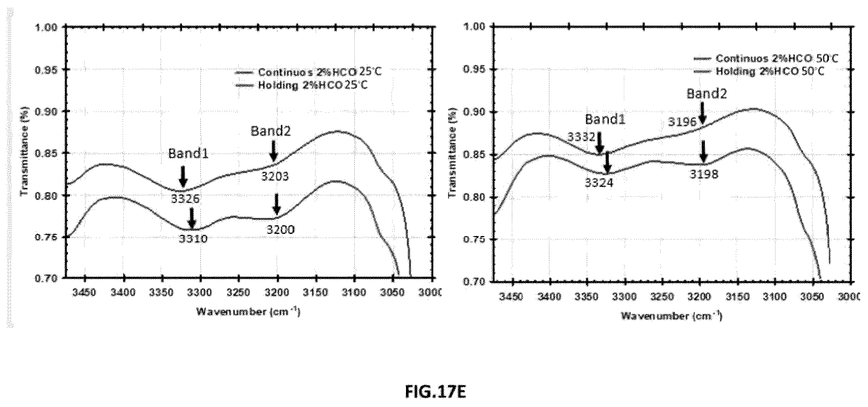

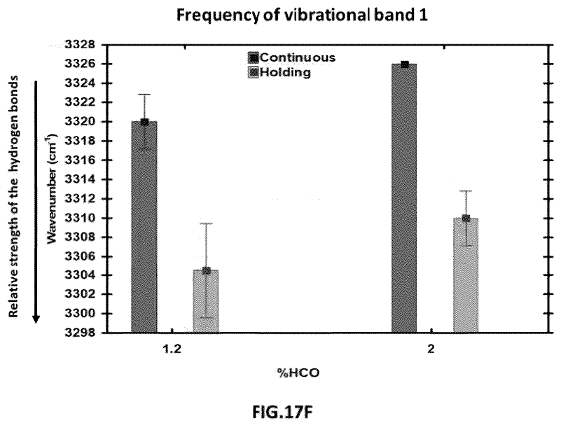

FTIR spectra measured for formulation's oils (Coconut oil+soybean oil+mineral oil+cyclomethicone), neat HCO and oils+1.2% HCO revealed the presence of hydrogen bonds (3350-3305 cm⁻¹ interval) when HCO was present (FIGS. 17A and 17B, green and orange lines). In FIG. 17A, the difference in intensities between the neat HCO and the other samples is due to the different accessory used for measurement (ATR for neat HCO and direct microscope slide observation for the other samples). In FIG. 17B, the wavenumber of the bands in the graph is the average from two replicates.

The area of interest is between 3100-3400 cm⁻¹ which is known to be one of the characteristic frequencies of stretching vibration from hydrogen bonds. Unfortunately, other vibrational bands of interest were overshadowed by the oils spectra. The neat HCO showed the presence of two bands in the 3150-3350 cm⁻¹ interval. These bands (associated with the presence of hydrogen bonding) were also present just in the systems with 1.2% HCO (Oils+1.2% HCO and 1.2% HCO holding). Absorbance peaks within the 3468-3435 cm⁻¹ interval (where oils showed some absorbance but not neat HCO; FIGS. 17A and 17B, blue and green lines), has been assigned to the overtone of the glyceride ester carbonyl vibration. (Sherazi et al., 2009; Guillen and Cabo, 2000).

FTIR spectra was measured through increasing temperatures for formulations with 1.2% HCO prepared in a holding process vs. a continuous heating-cooling process. In both formulations, as temperature increased, the O—H—O vibrational bands decreased as HCO reached its melting temperature where hydrogen bonds became weaker or not existent (FIG. 17C). A formulation prepared by a holding process showed sharper bands and a gradual decrease in bands through temperature increase as compared to a formulation prepared in a continuous heating-cooling process where the bands were shallower and the decrease in bands through temperature increase was steeper (FIG. 17C). These results demonstrated formulation prepared in a holding process had stronger hydrogen bonds that were less sensitive to increased temperature.

Comparison of the FTIR spectra of the two formulations at 25° C. and 50° C. showed the bands remained at 50° C. (FIG. 17D), indicating these bands correspond to HCO (as all other components of the formulation were melted below that temperature). In addition, analysis of the formulation prepared in a continuous heating-cooling process showed flattened bands at 50° C. as compared to the bands observed in a formulation prepared by a holding process that were sharper (FIG. 17D). The O—H vibrational bands were evident even when samples were heated up to 50° C. Since, at this temperature, all other waxes were in an isotropic state (i.e., melted), these bands corresponded to the HCO molecular self-assembly. Independent of the temperature, the O—H vibrational bands showed lower frequencies but with higher intensity for the holding process sample in comparison with the continuous process sample. This result indicated hydrogen bonds were weaker in a formulation prepared by a continuous heating-cooling process as they started to break around 50° C. compared to the bands in a formulation prepared by a holding process that decreased at a higher temperature.

Formulations containing 2% HCO prepared by a holding process or a continuous heating-cooling process showed bands of higher intensity as compared to formulations containing 1.2% HCO (FIG. 17E compared to FIG. 17D). Such an increase in bands intensity with increasing HCO concentrations corresponds to the presence of more inter-molecular bonds. Consequently, the differences in intensity and shape between samples (i.e., from continuous process vs from holding process) were more evident in the systems with 2% HCO. Average wavenumber of vibrations bands (1 and 2) were measured at 25° C. for the 1.2% HCO and 2% HCO systems manufactured with the holding or continuous process at the maximum peak.

| | 1.2% HCO | | 2% HCO | |
|---|---|---|---|---|
| wavenumber | Continuous | Holding | Continuous | Holding |
| Band 1 | 3320 | 3305 | 3326 | 3310 |
| | (±3) | (±5) | (±0) | (±3) |
| Band 2 | 3191 | 3195 | 3203 | 3200 |
| | (±6) | (±3) | (±2) | (±4) |

Note:
Results of two independent measurements. The standard deviation is reported within the brackets. Band 2 for the same % HCO was not significantly different between process at a P = 0.05.

Analysis of wavenumber absorption showed that band 2 remained the same for either process or HCO concentrations. However, for both HCO concentrations, in formulations prepared by a holding process, band 1 was detected at a lower wavenumber than that detected for formulations prepared by a continuous heating-cooling process (FIG. 17F). For band 2 there was no significant difference in the wavenumber vibration between process. However, for the holding process the band 1 occurred consistently at a lower wavenumber in the systems with 1.2% and 2% HCO.

Without being bound by theory, shifting to lower frequencies (wavenumbers) and higher intensities, e.g., for formulations prepared by a holding process, indicates stronger hydrogen bonding formed during this process. This result correlates with the higher melting temperature detected by DSC in formulations prepared by this process, as higher temperatures were required in order to break the association between molecules.

In one or more embodiments, the wavenumber of a formulation prepared by a holding process and stored at 25° C. is about 3301-3312 cm⁻¹. In one or more embodiments, the wavenumber of a formulation prepared by a holding process and stored at 50° C. is about 3320-3324 cm⁻¹.

### Example 18. Polarized Light Microscopy of FCD105 Placebo Sample

An FCD105 sample kept at 5° C. for 1 week was placed into an aluminum pan and observed through a polarized light microscope at room temperature and upwards until no crystals were observed while heating at 5° C./min. The light source used in the microscope was in reflection mode (light power arrangement from the top instead of the bottom, therefore light is being reflected by the DSC aluminum pan and through the sample).

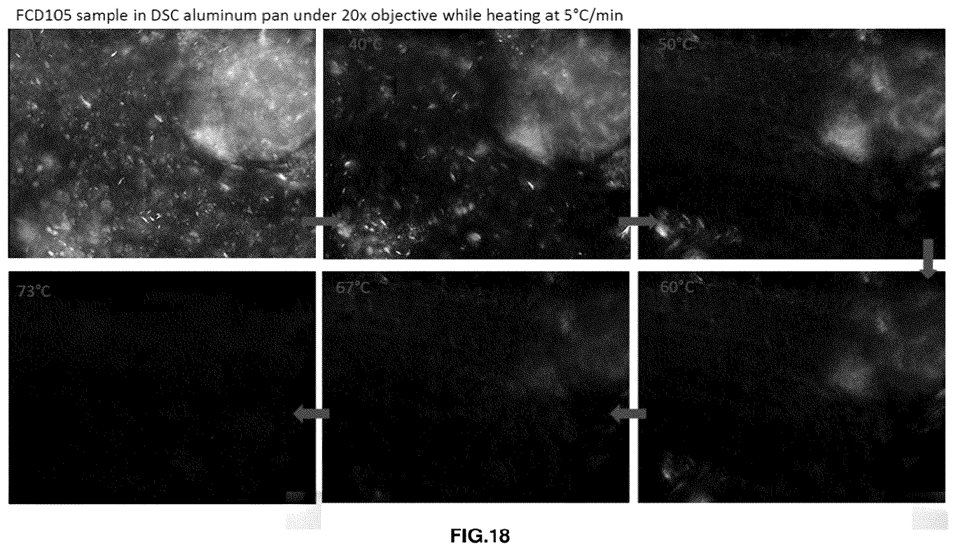

As can be seen in FIG. 18, only Tmh crystals were observed at 50° C. The Tmh crystal melting temperature was 73° C.

In the regular set up for microscopy analysis, where the sample is placed in glass slides, the Tmh crystals from this sample were observed to melt at about 86° C. This difference is likely because at higher temperature the solvents' (i.e. oils) viscosity decreases and flows through the glass slides. In contrast, it has been observed that the Tmh crystals adhere to the glass slide and remain in the same position while the oil flows away, altering the solubilization-melting properties and increasing the melting temperature of the crystals as if they were almost neat (powder without solvent). The does not happen in the DSC pan, since all of the sample remains, and therefore the Tmh crystals melt within the full system, closer to the DSC melting temperature TM=71.1° C.

### Example 19. Local and Systemic Toxicity and Toxicokinetics in Göttingen Minipigs Treated Topically with Either Vehicle Foam, Adapalene Foam or Minocycline and Adapalene Foams Prepared by a Holding Process

A study to characterize the local and systemic toxicity, and toxicokinetics of minocycline and adapalene foam with a fixed minocycline concentration of 3% and varying adapalene concentration of 0.1, 0.3, or 0.6%, along with adapalene as a comparator, topically administered to Göttingen Minipigs was performed as described in the Experimental Method section.

### Results

### Mortality

All animals survived to the scheduled necropsy.

### Detailed and Cageside Clinical Observations

Detailed and cageside clinical observations considered vehicle foam-, adapalene foam-, and/or minocycline and adapalene foam-related were all related to dermal findings, with the incidence for all increasing with time (Table 27). Being that the highest incidence for edema, erythema and scabbed area were in Groups 2 and 3, it is believed this was due to the nature of the vehicle foam, consisting mostly of oils. The reduced incidence for these calls in Groups 4 to 6 is believed to be due to the presence of the minocycline. Skin discolored red had a consistent incident rate across Groups 2 to 6, and in many cases included areas outside of the prescribed dose site. This observation was considered to be procedure-related. Erythema, scabbed area and skin discolored red were all observed by Week 3.

All of these dermal findings were considered to have reversed during the recovery phase and there were no anatomical or clinical pathology correlates.

TABLE 27
Summary of Detailed and Cageside Clinical Observation

| Group | | | | | | |
|---|---|---|---|---|---|---|
| No. | 1 | 2 | 3 | 4 | 5 | 6 |
| mg/kg/ | Un- | Vehicle | 0 Mino | 7.5 Mino | 7.5 Mino | 7.5 Mino |
| day | treated | Control | 0.75 | 0.25 | 0.75 | 1.50 |
| | | | Adap | Adap | Adap | Adap |

Edema

| | | | | | | |
|---|---|---|---|---|---|---|
| Males | 1/1 | 16/4 | 16/4 | 0/0 | 0/0 | 0/0 |
| Females | 0/0 | 14/3 | 22/6 | 0/0 | 2/2 | 0/0 |

Erythema

| | | | | | | |
|---|---|---|---|---|---|---|
| Males | 0/0 | 429/6 | 401/6 | 19/4 | 65/5 | 45/5 |
| Females | 0/0 | 464/6 | 436/6 | 17/2 | 72/6 | 79/6 |

Scabbed Area

| | | | | | | |
|---|---|---|---|---|---|---|
| Males | 34/2 | 248/6 | 107/6 | 9/1 | 67/5 | 44/3 |
| Females | 32/3 | 146/6 | 122/5 | 15/4 | 62/6 | 30/4 |

Skin Discolored Red

| | | | | | | |
|---|---|---|---|---|---|---|
| Males | 4/2 | 477/6 | 261/6 | 344/4 | 494/6 | 204/6 |
| Females | 0/0 | 560/6 | 380/6 | 489/4 | 653/6 | 587/6 |

Adap-Adapalene;
Mino-Minocycline;
Number of times observed/Total number of animals affected

The other clinical observations noted were not considered test article-related since they commonly occur in animals of this strain and age, were present at a low incidence or in controls, and/or were not dose-related.

### Dermal Irritation Scores

The dermal irritation scores were consistent with the detailed clinical observations. Erythema was called sporadically during the recovery period in a few males in Groups 2, 3, and 5.

### Body Weight and Body Weight Change

There were mild to moderate, reversible decreases in group mean body weight gain in both sexes and all groups compared to the untreated group, with the highest percent decreases in the vehicle-treated females, 7.5 minocycline/0.25 adapalene males and the 7.5 minocycline/1.50 adapalene females (see Text Table 28). Despite food enrichment being provided to some animals by Clinical Medicine, the animals were not noted as being thin. Since there were no clinical signs indicative of poor clinical condition or anatomical/clinical pathology correlates, a direct relationship to the vehicle, adapalene or minocycline and adapalene foams could not be determined.

TABLE 28
Percent Difference in Group Mean Body Weight Gain from Day
−1 to the End of the Dose Phase, as Compared to Untreated

| Group No. | 2 | 3 | 4 | 5 | 6 |
|---|---|---|---|---|---|
| mg/kg/day | Vehicle | 0 Mino | 7.5 Mino | 7.5 Mino | 7.5 Mino |
| | Control | 0.75 Adap | 0.25 Adap | 0.75 Adap | 1.50 Adap |
| Males | −19 | −3 | −52 | −25 | −39 |
| Females | −53 | −31 | −35 | −44 | −51 |

Adap - Adapalene;
Mino - Minocycline

### Ophthalmic Examination

There were no findings related to vehicle, minocycline and adapalene, or adapalene foam treatments. The observations noted were representative of pathology that would be expected for this group of animals considering age, sex, and strain; no obvious trends in pathology would suggest that test material-related reactions had occurred.

### Electrocardiography Report

A board-certified veterinary cardiologist conducted a qualitative and quantitative review of the electrocardiograms obtained pre-dose and one to two hours post-dose following dermal administration of adapalene foam, minocycline and adapalene foam (three dose strengths), vehicle foam or no treatment and following a recovery period in designated animals. There was no effect of the dermal administration of adapalene foam or minocycline and adapalene foam on qualitative ECG parameters. There was a mild slowing of the heart rate and lengthening of the RR interval at the terminal post-dose interval following high-dose minocycline and adapalene foam treatment that persisted at the recovery interval. The changes relative to changes following vehicle foam and no treatment were mild and not considered adverse.

### Clinical Pathology

### Summary

There were no minocycline and adapalene or adapalene-related effects among clinical pathology parameters in minipigs of either sex following repeated dermal administration of minocycline and adapalene or adapalene foam, with the exception of non-adverse decrease in alkaline phosphatase (ALP) on Day 92 in minocycline and adapalene high dose females (Group 6).

### Hematology

There were no minocycline and adapalene—or adapalene related effects among hematology parameters. All fluctuations among individual and mean values were considered sporadic, consistent with biologic variation and/or negligible in magnitude, and not related to minocycline and adapalene or adapalene foam administration.

### Coagulation

There were no minocycline and adapalene—or adapalene-related effects among coagulation parameters. All fluctuations among individual and mean values were considered sporadic, consistent with biologic variation and/or negligible in magnitude, and not related to minocycline and adapalene or adapalene foam administration.

### Clinical Chemistry

There were no minocycline and adapalene—or adapalene-related effects, except for the non-adverse decrease in ALP on Day 92 in minocycline and adapalene high-dose females (Group 6). All other fluctuations among individual and mean values were considered sporadic, consistent with biologic variation and/or negligible in magnitude, and not related to minocycline and adapalene or adapalene foam administration.

### Urinalysis

No minocycline and adapalene—or adapalene-related alterations were observed among urinalysis parameters. There were occasional differences found in urine volume and specific gravity that were not considered toxicologically meaningful due to their sporadic nature and the inherent variability of these endpoints. There were some variations between treatment groups among physical (appearance) and biochemical, urinary components; however, all findings were considered within expected range for biological and/or procedure-related variability.

### Bioanalysis and Toxicokinetic Evaluation

Conclusion for both Adapalene and Minocycline Bioanalysis

The accuracy and precision data are comparable to the results obtained during the validation for minipig plasma. The requirements for project acceptance, as defined in the bioanalytical protocol, were fulfilled. Incurred sample reanalysis (ISR) experiments revealed an acceptable reproducibility of the applied assay. It is concluded that the sample results are reliable within the given accuracy and precision range.

### Toxicokinetic Evaluation (see Text Tables 29, 30, and 31)

Systemic exposure to adapalene and minocycline appeared to be independent of sex on Day 91.

0.75 mg/kg Adapalene (Group 3)
Systemic exposure (AUC₀₋₂₄ₕᵣ) to adapalene appeared to increase following repeated dermal administration of adapalene.
   Adapalene—0.25, 7.5 and 1.5 mg/kg adapalene in combination with 7.5 mg/kg minocycline (minocycline and adapalene—Group 4, 5 and 6, respectively)
Following daily dermal administration of minocycline and adapalene (increasing dose of adapalene in combination with fixed minocycline dose), mean Cₘₐₓ and AUC₀₋₂₄ₕᵣ values for adapalene increased with increasing dose in an approximately dose proportional manner from 0.25 to 1.5 mg/kg on Day 91.
Systemic exposure (AUC₀₋₂₄ₕᵣ) to adapalene increased following repeated dermal administration of minocycline and adapalene (increasing dose of adapalene in combination with minocycline) at 0.75 and 1.5 mg/kg.
   Adapalene—Group 3 (0.75 mg/kg adapalene alone) vs Group 5 (0.75 mg/kg adapalene in combination 7.5 mg/kg minocycline)
   Systemic exposure to adapalene appeared similar for Group 5 when compared to Group 3 on Day 91.

Adapalene—0.25, 7.5 and 1.5 mg/kg adapalene in combination with 7.5 mg/kg minocycline (FCD105—Group 4, 5 and 6, respectively)
Systemic exposure (AUC₀₋₂₄ₕᵣ) to adapalene appeared to increase following repeated dermal administration of FCD105 (increasing dose of adapalene in combination with minocycline) for Group 6.
Systemic exposure (AUC₀₋₂₄ₕᵣ) to minocycline appeared similar between Group 4, 5, and 6 on Day 91.

TABLE 29
Mean (±SD) and CV % Adapalene Toxicokinetic Parameters on Days 1 and 91
Following Daily Dermal Administration of 0.75 mg/kg Adapalene to Minipigs (Males
and Females Combined)

| | | | | | Cₘₐₓ | Cₘₐₓ/Dose | | | AUC_{Tlast} | AUC₀₋₂₄ₕᵣ | AUC₀₋₂₄ₕᵣ/Dose | |
|---|---|---|---|---|---|---|---|---|---|---|---|---|
| | | Dose | | | (pg/ | (kg · pg/ | Tₘₐₓª | Tₗₐₛₜª | (hr · pg/ | (hr · pg/ | (hr · kg · pg/ | |
| Analyte | Group | (mg/kg) | Day | Statistic | mL) | mL/mg) | (hr) | (hr) | mL) | mL) | mL/mg) | R^{b} |
| Adapalene | 3 | 0.75 | 1 | N | 12 | 4 | 4 | 4 | 1 | 1 | 1 | NA |
| | | | | Mean | 97.1 | 388 | 24 | 24 | 7730 | 7730 | 10300 | NA |
| | | | | SD | 151 | 121 | (24-24) | (24-24) | NA | NA | NA | NA |
| | | | | CV % | 156 | 31.2 | NA | NA | NA | NA | NA | NA |
| Adapalene | 3 | 0.75 | 91 | N | 12 | 12 | 12 | 12 | 12 | 12 | 12 | 1 |
| | | | | Mean | 4500 | 5990 | NA | 24 | 104000 | 69200 | 92300 | 8.29 |
| | | | | SD | 1940 | 2580 | (0-8) | (24-504) | 56200 | 35200 | 47000 | NA |
| | | | | CV % | 43.1 | 43.1 | NA | NA | 54.1 | 50.9 | 50.9 | NA |

NA-Not applicable.
SD and CV are not calculated or not reported when mean concentration equals zero or N < 3.
^{a}Median (minimum-maximum), median value only reported if actual collection interval.
^{b}R = AUC_{0-24hr Day 91}/AUC_{0-24hr Day 1}.

TABLE 30
Mean (±SD) and CV % Adapalene Toxicokinetic Parameters on Days 1 and 91 Following Daily Dermal
Administration of 0.25, 0.75, and 1.50 mg/kg Adapalene in combination with 7.5 mg/kg Minocycline
FCD105 Foam to Minipigs (Males and Females Combined)

| | | | | | Cₘₐₓ | Cₘₐₓ/Dose | | | AUC_{Tlast} | AUC₀₋₂₄ₕᵣ | AUC₀₋₂₄ₕᵣ/Dose | |
|---|---|---|---|---|---|---|---|---|---|---|---|---|
| | | Dose | | | (pg/ | (kg · pg/ | Tₘₐₓª | Tₗₐₛₜª | (hr · pg/ | (hr · pg/ | (hr · kg · pg/ | |
| Analyte | Group | (mg/kg) | Day | Statistic | mL) | mL/mg) | (hr) | (hr) | mL) | mL) | mL/mg) | R^{b} |
| Adapalene | 4 | 0.25 | 1 | N | 8 | 2 | 2 | 2 | NA | NA | NA | NA |
| | | | | Mean | 31.4 | 502 | NA | NA | NA | NA | NA | NA |
| | | | | SD | 58.6 | NA | (8-24) | (8-24) | NA | NA | NA | NA |
| | | | | CV % | 187 | NA | NA | NA | NA | NA | NA | NA |
| Adapalene | 4 | 0.25 | 91 | N | 8 | 8 | 8 | 8 | 8 | 8 | 8 | NA |
| | | | | Mean | 2800 | 11200 | NA | 24 | 31600 | 31600 | 126000 | NA |
| | | | | SD | 2530 | 10100 | (0-24) | (24-24) | 24200 | 24200 | 96800 | NA |
| | | | | CV % | 90.5 | 90.5 | NA | NA | 76.5 | 76.5 | 76.5 | NA |
| Adapalene | 5 | 0.75 | 1 | N | 12 | 6 | 6 | 6 | 1 | 1 | 1 | NA |
| | | | | Mean | 72.2 | 192 | 24 | 24 | 3180 | 3180 | 4230 | NA |
| | | | | SD | 78.4 | 42.9 | (8-24) | (24-24) | NA | NA | NA | NA |
| | | | | CV % | 109 | 22.3 | NA | NA | NA | NA | NA | NA |
| Adapalene | 5 | 0.75 | 91 | N | 12 | 12 | 12 | 12 | 12 | 12 | 12 | 1 |
| | | | | Mean | 6870 | 9160 | 1 | 24 | 91400 | 91400 | 122000 | 40.7 |
| | | | | SD | 3860 | 5140 | (0-8) | (24-24) | 39000 | 39000 | 52100 | NA |
| | | | | CV % | 56.1 | 56.1 | NA | NA | 42.7 | 42.7 | 42.7 | NA |
| Adapalene | 6 | 1.50 | 1 | N | 12 | 6 | 6 | 6 | 3 | 3 | 3 | NA |
| | | | | Mean | 480 | 640 | 24 | 2 | 28800 | 28800 | 19200 | NA |
| | | | | SD | 1120 | 985 | (8-24) | (24-24) | 34000 | 34000 | 22700 | NA |
| | | | | CV % | 232 | 154 | NA | NA | 118 | 118 | 118 | NA |
| Adapalene | 6 | 1.50 | 91 | N | 12 | 12 | 12 | 12 | 12 | 12 | 12 | 3 |
| | | | | Mean | 10500 | 6970 | 1 | 24 | 118000 | 118000 | 78700 | 11.2 |
| | | | | SD | 9200 | 6130 | (0-8) | (24-24) | 79000 | 79000 | 52700 | 11.0 |
| | | | | CV % | 88.0 | 88.0 | NA | NA | 66.9 | 66.9 | 66.9 | 98.2 |

NA-Not applicable.
SD and CV are not calculated or not reported when mean concentration equals zero or N < 3.
^{a}Median (minimum-maximum), median value only reported if actual collection interval.
^{b}R = AUC_{0-24hr Day 91}/AUC_{0-24hr Day 1}.

TABLE 31
Mean (±SD) and CV % Minocycline Toxicokinetic Parameters on Days 1 and 91 Following Daily Dermal Application of
7.5 mg/kg Minocycline in combination with 0.25, 0.75, and 1.50 mg/kg Adapalene FCD105 Foam to Minipigs (Males and
Females Combined)

| | | | | | | Cₘₐₓ/Dose | | | AUC_{Tlast} | AUC₀₋₂₄ₕᵣ | AUC₀₋₂₄ₕᵣ/Dose | |
|---|---|---|---|---|---|---|---|---|---|---|---|---|
| | | Dose | | | Cₘₐₓ | (kg · ng/mL/ | Tₘₐₓª | Tₗₐₛₜª | (hr · ng/ | (hr · ng/ | (hr · kg · ng/ | |
| Analyte | Group | (mg/kg) | Day | Statistic | (pg/mL) | mg) | (hr) | (hr) | mL) | mL) | mL/mg) | R^{b} |
| Minocycline | 4 | 7.5 | 1 | N | 8 | 3 | 3 | 3 | 1 | NA | NA | NA |
| | | | | Mean | 0.314 | 0.112 | 4 | 4 | 7.69 | NA | NA | NA |
| | | | | SD | 0.492 | 0.0581 | (2-4) | (2-8) | NA | NA | NA | NA |
| | | | | CV % | 157 | 52.1 | NA | NA | NA | NA | NA | NA |
| Minocycline | 4 | 7.5 | 91 | N | 8 | 8 | 8 | 8 | 8 | 8 | 8 | NA |
| | | | | Mean | 8.76 | 1.17 | 2 | 24 | 98.6 | 98.6 | 13.2 | NA |
| | | | | SD | 7.32 | 0.976 | (0-4) | (24-24) | 55.7 | 55.7 | 7.43 | NA |
| | | | | CV % | 83.6 | 83.6 | NA | NA | 56.5 | 56.5 | 56.5 | NA |
| Minocycline | 5 | 7.5 | 1 | N | 12 | 1 | 1 | 1 | NA | NA | NA | NA |
| | | | | Mean | 0.0491 | 0.0785 | 24 | 24 | NA | NA | NA | NA |
| | | | | SD | 0.170 | NA | NA | NA | NA | NA | NA | NA |
| | | | | CV % | 346 | NA | NA | NA | NA | NA | NA | NA |
| Minocycline | 5 | 7.5 | 91 | N | 12 | 12 | 12 | 12 | 12 | 12 | 12 | NA |
| | | | | Mean | 12.4 | 1.65 | 2 | 24 | 152 | 152 | 20.3 | NA |
| | | | | SD | 5.45 | 0.727 | (1-4) | (24-24) | 41.9 | 41.9 | 5.59 | NA |
| | | | | CV % | 44.0 | 44.0 | NA | NA | 27.6 | 27.6 | 27.6 | NA |
| Minocycline | 6 | 7.5 | 1 | N | 12 | 2 | 2 | 2 | 2 | 1 | 1 | NA |
| | | | | Mean | 0.612 | 0.489 | NA | NA | 48.6 | 89.7 | 12.0 | NA |
| | | | | SD | 1.75 | NA | (4-8) | (8-24) | NA | NA | NA | NA |
| | | | | CV % | 287 | NA | NA | NA | NA | NA | NA | NA |
| Minocycline | 6 | 7.5 | 91 | N | 12 | 12 | 12 | 12 | 12 | 12 | 12 | 1 |
| | | | | Mean | 17.4 | 2.32 | NA | 24 | 167 | 167 | 22.3 | 2.75 |
| | | | | SD | 11.3 | 1.51 | (0-8) | (24-24) | 67.0 | 67.0 | 8.93 | NA |
| | | | | CV % | 65.1 | 65.1 | NA | NA | 40.1 | 40.1 | 40.1 | NA |

NA-Not applicable.
SD and CV are not calculated or not reported when mean concentration equals zero or N < 3.
^{a}Median (minimum-maximum), median value only reported if actual collection interval.
^{b}R = AUC_{0-24hr Day 91}/AUC_{0-24hr Day 1}.

Terminal Evaluations

Gross Pathology

Terminal Euthanasia Animals (Day 92)

No minocycline and adapalene- or adapalene-related gross findings were noted. The gross findings observed were considered incidental, of the nature commonly observed in this strain and age of minipig, and/or were of similar incidence in control and treated animals and, therefore, were considered unrelated to administration of minocycline and adapalene or adapalene foam.

Recovery Euthanasia Animals (Day 120)

No minocycline and adapalene—or adapalene-related gross findings were noted. The gross findings observed were considered incidental, of the nature commonly observed in this strain and age of minipig and/or were of similar incidence in control and treated animals and, therefore, were considered unrelated to administration of minocycline and adapalene or adapalene foam.

Organ Weights

Terminal Euthanasia Animals (Day 92)

No minocycline and adapalene—or adapalene-related organ weight changes were noted. There were isolated organ weight values that were statistically different from their respective controls. There were, however, no patterns, trends, or correlating data to suggest these values were toxicologically relevant. Thus, the organ weight differences observed were considered incidental and/or related to difference of sexual maturity and unrelated to administration of minocycline and adapalene or adapalene foam.

Recovery Euthanasia Animals (Day 120)

Due to small sample size (N<3), tests of statistical significance were not performed for this time point. No minocycline and adapalene—or adapalene-related organ weight changes were noted. There were isolated organ weight values that were different from their respective controls. There were, however, no patterns, trends, or correlating data to suggest these values were toxicologically relevant. Thus, the organ weight differences observed were considered incidental and/or related to difference of sexual maturity and unrelated to administration of minocycline and adapalene or adapalene foam.

Histopathology

Terminal Euthanasia (Day 92)

No minocycline and adapalene—or adapalene-related microscopic findings were noted. The microscopic findings observed were considered incidental, of the nature commonly observed in this strain and age of minipig, and/or were of similar incidence and severity in control and treated animals and, therefore, were considered unrelated to administration of minocycline and adapalene or adapalene foam.

Recovery Euthanasia (Day 120)

No minocycline and adapalene—or adapalene-related microscopic findings were noted, and no target tissues were identified at terminal sacrifice, therefore, microscopic evaluation was limited to evaluation of gross lesions. The microscopic findings observed were considered incidental, of the nature commonly observed in this strain and age of minipig, and/or were of similar incidence in control and treated animals and, therefore, were considered unrelated to administration of minocycline and adapalene or adapalene foam.

### CONCLUSIONS

Once daily dermal administration of minocycline and adapalene foam to minipigs for up to 91 days at doses of untreated, 0/0, 0/0.75, 7.5/0.25, 7.5/0.75 or 7.5/1.5 mg/kg/day minocycline/adapalene was well tolerated at all dose levels. There were reversible minimal to moderate treatment-related dermal irritation that was considered to be vehicle foam related. Thus, the no-observed-adverse-effect-level (NOAEL) is considered to be the 7.5/1.5 mg/kg/day minocycline/adapalene dose. On Day 91, combined-sex mean Cmax and AUC0-24 hr values were 17.4 ng/mL and 167 hr·ng/mL for 3% minocycline and 10,500 pg/mL and 118,000 hr·pg/mL for 0.6% adapalene

### Example 20

### Example of a Clinical Study in Patients with Moderate-to-Severe Acne Vulgaris

Study Synopsis:

Title of Study: A Prospective, Multicenter, Randomized, Double-Blind, Vehicle-Controlled Phase 2 Study to Evaluate the Safety and Efficacy of a Combination of 3% Minocycline and 0.3% Adapalene Topical Foam Formulation for the Treatment of Moderate-to-Severe Acne (Study FX2016-40).

Objectives:

To evaluate the safety, tolerability, and efficacy of the combination product MAH in the treatment of moderate-to-severe acne vulgaris with up to 12 weeks of daily treatment, in comparison to the vehicle.

To compare the efficacy and safety of MAH (e.g., 200426D or 190505S) to the individual active drug components: minocycline 3% and adapalene 0.3% topical foam products.

Name of Active Ingredients: Minocycline hydrochloride and adapalene.

Study Design and Methods: This is a randomized, multicenter, double-blind, vehicle-controlled study to evaluate the safety, tolerability, and efficacy over a 12-week treatment period of MAH compared to its active components and matched color vehicle foam in the treatment of subjects with moderate-to-severe acne vulgaris.

Subjects with qualifying lesion counts (inflammatory and non-inflammatory acne lesions) and Investigator's Global Assessment (IGA) of acne severity scores are assigned to 1 of the following 4 treatments according to the randomization schedule:
MAH (minocycline 3%+adapalene 0.3%)
Vehicle (coloring added to match the color of MAH)
Minocycline 3%
Adapalene 0.3% (coloring added to match the color of MAH)

Subjects are instructed to apply the assigned study drug topically once daily approximately 1 hour before bedtime to the full face and other acne-affected areas of the body for 12 weeks as directed. Subjects are advised to apply the study drug at approximately the same time each day. Both the Investigator and subject are blinded to the study drug identity.

Following the Baseline visit, subjects are to return for visits at Weeks 4, 8, and 12 (Table 9A). If a treatment-emergent adverse event (TEAE) is reported at Visit 4 (Week 12/End of Treatment (EOT)), that subject is to return for a follow-up visit 1 month after treatment termination (Visit 5, Week 16) for a safety evaluation. If the subject is unable to return for a visit a follow-up phone call should be conducted.

The co-primary efficacy evaluations (lesion counts and IGA) is performed at Baseline and at Weeks 4, 8, and 12 during the study. Other efficacy assessments are performed as described in the protocol. Safety and tolerability evaluations are performed at every visit.

Number of Subjects (planned): The planned enrollment is approximately 400 subjects, as follows: 125 subjects in the MAH arm; 100 subjects each in the minocycline 3% foam arm and the adapalene 0.3% foam arm; and 75 subjects in the vehicle arm.

Diagnosis and Main Criteria for Inclusion: Healthy male or non-pregnant females, aged ≥12 years, with a clinical diagnosis of moderate-to-severe facial acne vulgaris characterized by:
20 to 50 inflammatory lesions (papules and/or pustules) on the face;
25 to 100 non-inflammatory lesions (open and closed comedones) on the face;
IGA score of moderate (3) to severe (4); and
Subjects are to have no more than two active nodules on the face.

Test Products, Doses and Modes of Administration: MAH (minocycline 3%+adapalene 0.3%) foam, topical application; minocycline 3% foam, topical application; adapalene 0.3% foam, topical application. All study treatments are applied once daily to the full face and other acne-affected areas of the body for 12 weeks.

Reference Therapy: Vehicle foam, topical application, once daily to the full face and other acne-affected areas of the body, for 12 weeks.

Study Duration: Subject participation in the study is up to 22 weeks: up to 6 weeks for Screening and Baseline, 12 weeks of study treatment, and up to 4 weeks of follow-up.

Endpoints and Outcomes:

Efficacy Evaluations

The efficacy assessments include inflammatory and non-inflammatory lesion counts and the IGA at Baseline and at Weeks 4, 8, and 12, as well as other secondary efficacy measures.

The co-Primary efficacy endpoints are:
The absolute change from Baseline in inflammatory lesion count at Week 12.
The absolute change from Baseline in non-inflammatory lesion count at Week 12.
Proportion of subjects (%) with an IGA score of 0/1 (“Clear” or “Minimal”) and at least a 2-grade improvement (decrease) at Week 12.

The secondary efficacy endpoints are:
Percent change from Baseline in the inflammatory and non-inflammatory lesion counts at Weeks 12, 8, and 4.
IGA Treatment Success: IGA score of 0 or 1 and at least a 2-grade improvement (decrease) at Weeks 8 and 4
IGA Treatment Success: at least a 2-grade improvement (decrease) in IGA score at Week 12
FCD105 vs. 0.3% adapalene foam, and FCD105 vs. 3% minocycline foam for all co-primary endpoints (inflammatory/non-inflammatory count/IGA treatment success) at Week 12.

Safety Evaluations

The safety assessments include TEAEs (volunteered, observed, and elicited by general questioning in a non-suggestive manner), physical examinations, vital signs, and local skin tolerability assessments (including itching, stinging/burning, dryness, scaling, erythema, and hyperpigmentation).

Statistical Methods:

Primary efficacy analyses are conducted on the intent-to-treat (ITT) population using the Multiple Imputation approach. MAH is tested against vehicle at the two-sided 0.05 level of significance without adjustment for multiplicity. The absolute change from Baseline in inflammatory and non-inflammatory lesions at Visit 4 (Week 12/End of Treatment) is analyzed using an analysis of covariance (ANCOVA) model with the main effect of treatment and corresponding baseline lesion count and investigational site as covariates. IGA Treatment Success is analyzed using a Cochran-Mantel-Haenszel (CMH) test stratified by site. The co-primary efficacy analyses are:
Test for statistical superiority of MAH vs. vehicle in the absolute change from Baseline in inflammatory lesion counts at Week 12.
Test for statistical superiority of MAH vs. vehicle in the absolute change from Baseline in non-inflammatory lesion counts at Week 12.
Test for statistical superiority of MAH vs. vehicle in IGA Treatment Success (dichotomized as Yes/No) at Week 12, where success is defined as an IGA score of 0 or 1, and at least a 2-grade improvement (decrease) from Baseline.

Supportive efficacy analyses are also conducted on the Per Protocol (PP) population; all analyses using the PP population use the Observed Cases approach and there is no imputation for missing data at any time point. Sites with a small number of subjects are pooled together for all analyses stratified by site.

Secondary and other analyses continuous endpoints are analyzed using ANCOVA models. Categorical endpoints are analyzed using the CMH test stratified by site. Time to event endpoints are analyzed using Kaplan-Meier survival plots and a log-rank test stratified by site.

No statistical tests are performed for any of the safety assessments.

Inclusion Criteria

A male or female subject are considered eligible for participation in the study if all of the following inclusion criteria are satisfied prior to randomization:
1. Has completed and signed an appropriately administered Informed Consent Form (ICF) prior to any study-related procedures. Subjects less than 18 years of age (or as required by state law) must sign an Assent Form for the study and a parent or legal guardian must sign the ICF.
2. Is 12 years of age or older.
3. Has facial acne vulgaris with all of the following:
a. 20 to 50 inflammatory lesions (papules and/or pustules) on the face.
b. 25 to 100 non-inflammatory lesions (open and closed comedones) on the face.
c. IGA score of moderate (3) to severe (4).
4. No more than two active nodules on the face.
5. If a female of child-bearing potential:
a. A negative urine pregnancy test.
b. Agreement to use an effective method of contraception, if the subject is sexually active or becomes sexually active, for the entire study period.
6. If the subject is using a hormonal contraceptive, she must have been on the same type and strength over the last 3 months prior to study entry.
7. Willing to use only the supplied non-medicated cleanser and to refrain from use of any other acne medication, medicated cleanser, excessive sun exposure, and tanning booths for the duration of the study.

Exclusion Criteria

Subjects who fulfill any of the following criteria are not recruited into the study:
1. A female who is pregnant or lactating, or is planning a pregnancy during the study.
2. Acne conglobata, acne fulminans, secondary acne (chloracne, drug-induced acne), or any dermatological condition of the face that could interfere with the clinical evaluations.
3. Facial hair (e.g., beard, mustache, etc.) that could interfere with the clinical evaluations.
4. Sunburn on the face.
5. Severe systemic disease as assessed by the Investigator that might interfere with the conduct of the study or the interpretation of the results.
6. Currently participating, or has participated within 30 days prior to this study, in an investigational drug or device study.
7. Inability to fully comply with the study requirements. Participation in photography is not a study requirement.
8. Subjects who have a documented history of any of the following:
a. Allergy to tetracycline-class antibiotics or to any ingredient in the study drug.
b. Pseudomembranous colitis or antibiotic-associated colitis.
c. Hepatitis or clinically significant liver damage or clinically significant renal impairment.
d. Known or suspected premalignant or malignant disease (excluding successfully treated skin cancers).
9. Subjects who have used the following medications:
   Within 1 week prior to randomization:
Medicated facial cleansers on the face.
Topical acne treatments on the face (other than those listed below).
   Within 4 weeks prior to randomization:
Topical retinoids on the face.
   Topical anti-inflammatories and/or corticosteroids on the face.
Topical corticosteroids on body areas other than the face for more than 15 consecutive days and on more than 10% of the body surface area. In body folds, such as axillary and inguinal regions, only mild topical steroids are allowed for short term use (≤15 consecutive days).
Systemic antibiotics.
Systemic acne treatments.
   Within 12 weeks prior to randomization:
Systemic retinoids.
Systemic corticosteroids (Note: Intranasal and inhaled corticosteroids may be used throughout the study if the subject is on a stable dose).
10. Use of sauna during the 2 weeks prior to randomization.
11. Epilation of the face within 2 weeks prior to randomization.
12. Folliculitis on the face.
13. Documented drug addiction or alcohol abuse within the last 2 years. Heavy drinking levels defined by. The Substance Abuse and Mental Health Services Administration (SAMHSA) as drinking 5 or more alcoholic drinks on the same occasion on each of 5 or more days in the past 30 days.
14. Documented history of depression that is not, in the opinion of the Investigator, currently adequately controlled with medication.

A summary of study assessments and the time point at which they are to be performed during the study is presented in Table 32.

TABLE 32
Schedule of Study Assessments and Procedures, Protocol FX2016-40

| | | | | | | End of | |
|---|---|---|---|---|---|---|---|
| | | | | | | Treatment | |
| | | | | | | Week | Follow- |
| | | | | | | 12³ | Up⁸ |
| | | | Week 2 | Week 4 | Week 8 | (±4 | Week 16 |
| Activity | Screening¹ | Baseline² | Phone Call | (±4 days) | (±4 days) | days) | (±4 days) |
| Visit No | | 1 | | 2 | 3 | 4 | 5 |
| Informed Consent/Assent | X | | | | | | |
| Assign Subject | X | | | | | | |
| Identification Number | | | | | | | |
| Inclusion/Exclusion | X | X | | | | | |
| Criteria | | | | | | | |
| Demographic Data | X | | | | | | |
| Medical/Surgical | X | | | | | | |
| History and | | | | | | | |
| Medication | | | | | | | |
| (prior/concomitant)¹⁰ | | | | | | | |
| Physical | | X | | | | X | |
| Examination, incl. | | | | | | | |
| height and weight⁴ | | | | | | | |
| Blood Pressure/Heart | X | X | | X | X | X | |
| Rate⁵ | | | | | | | |
| Urine Pregnancy | X | X | | X | X | X | |
| Test⁶ | | | | | | | |
| Investigator's Global | X | X | | X | X | X | |
| Assessment | | | | | | | |
| Lesion Count | X | X | | X | X | X | |
| (inflammatory/non- | | | | | | | |
| inflammatory) | | | | | | | |
| Compliance Phone | | | X | | | | |
| Call | | | | | | | |
| Subject Satisfaction | | X⁷ | | | | X⁷ | |
| Questionnaire | | | | | | | |
| Photography | | X | | X | X | X | |
| (participating sites | | | | | | | |
| only) | | | | | | | |
| Randomization | | X | | | | | |
| Concomitant | | X | X | X | X | X | X |
| Medications | | | | | | | |
| Adverse Events | | X | X | X | X | X | X |
| Tolerability | | X | | X | X | X | |
| (erythema, dryness, | | | | | | | |
| burning/stinging, | | | | | | | |
| hyperpigmentation, | | | | | | | |
| scaling, itching) | | | | | | | |
| Dispense Study Drug | | X | | X | X | | |
| Dispense Cleanser | X⁹ | X | | X | X | | |
| (as needed) | | | | | | | |
| Collect Used Study | | | | X | X | X | |
| Drug Canisters and | | | | | | | |
| Perform Drug | | | | | | | |
| Accountability | | | | | | | |
| Distribute Study Drug | | X | | | | | |
| Application Guideline | | | | | | | |
| Confirm Next Visit | X | X | X | X | X | X | |

¹The maximum duration of Screening is up to 6 weeks. If there are medications to be discontinued it will not be less than the time indicated in exclusion criterion 9.
²Baseline and the procedures performed at this visit can be combined with Screening. Assessments scheduled for the Screening and Baseline visits do not need to be duplicated in case the Screening and Baseline visits are combined. However, study drug is not dispensed until it has been verified that all inclusion criteria and none of the exclusion criteria are met. If Screening and Baseline visits are not combined, the Baseline visit will occur within 6 weeks of Screening.
³If a subject prematurely withdraws from the study, all evaluations described under Visit 4 (Week 12/End of Treatment Visit) are performed.
⁴Measure height at Baseline. Record weight at Baseline and at Visit 4 (Week 12/End of Treatment Visit).
⁵Measure blood pressure and heart rate after the subject has been sitting for at least 5 minutes at rest.
⁶Urine samples for pregnancy testing are obtained at each study visit (including an Early Termination visit) from female subjects. Urine pregnancy testing is performed at the sites.
⁷Subject Satisfaction Questionnaire, questions 1 and 2 only should be answered following the IGA and before the lesion counts. The subject is not told their IGA rating of the acne.
⁸The Follow-Up visit is conducted for subjects that report a treatment-emergent adverse event (TEAE) at Visit 4 (Week 12/End of Treatment Visit). If the subject is unable to show up for the visit a follow-up phone call should be conducted. Adverse events that are ongoing when a subject withdraws from or completes the study are followed up until resolution or stabilization, or for 30 days, whichever is shorter.
⁹Non-medicated cleanser are dispensed at Screening to subjects that fulfill the inclusion and exclusion criteria and indicate an intent to participate in the study. After Screening it is dispensed as needed.
¹⁰Prior/Concomitant medication taken within 3 months of screening is recorded.

### Study Drug Kits and Treatments, Protocol FX2016-40

| | |
|---|---|
| Dosage form | Active kit: MAH (minocycline 3% + adapalene |
| descriptions: | 0.3%) topical foam |
| | Active control kit: Minocycline 3% topical foam |
| | Active control kit: Adapalene 0.3% topical foam |
| | (color matched) Vehicle kit: Vehicle topical foam |
| | (color matched) |
| Package | All study drug products are supplied as identical |
| description: | canisters containing 30 g foam. The product is |
| | dispensed as foam when the actuator is pressed |
| | down. |
| Daily dose: | 105.7 mg of minocycline and 10.6 mg adapalene |
| | are applied daily. |
| Cumulative | Based on the estimated upper range of foam to |
| maximal dose: | be used daily of 3523.1 mg (3.5 g), 12 weeks (84 |
| | days) of once daily treatment would provide an |
| | accumulated dose of approximately 8878 mg (8.9 |
| | g) minocycline and 887.8 mg (0.89 g) adapalene. |
| Dispensing: | One kit (i.e., 2 canisters) is dispensed to each |
| | subject at each of the following visits: Baseline |
| | (Visit 1), Visit 2 and Visit 3. A total of 3 kits (6 |
| | canisters) is dispensed to each subject during the |
| | study. In case the supplied study treatment |
| | becomes unavailable to the subject (e.g., is lost, |
| | etc.) a new kit containing the same treatment |
| | regimen may be supplied by the Investigator (or |
| | designee) and documented accordingly. |

### Efficacy Assessments

Co-Primary Efficacy Assessments

The co-primary efficacy assessments include inflammatory and non-inflammatory lesion counts and the IGA of severity of disease.

Investigator Global Assessment

Table 33 shows the IGA scale for acne vulgaris, which is used to assess the severity of a subject's acne vulgaris. The IGA is a static evaluation of global severity representing clinically meaningful graduations of the disease. The IGA is performed prior to the lesion count. The IGA is performed as specified in Table 33.

TABLE 33
IGA Scale for Acne Vulgaris, Protocol FX2016-40

| Score | Grade | Description |
|---|---|---|
| 0 | Clear | Clear skin with no papules or pustules (inflammatory lesions) or |
| | | comedones (non-inflammatory lesions). Post-inflammatory |
| | | hyperpigmentation is allowable. |
| 1 | Minimal | Few non-inflammatory lesions (comedones) are present; and up |
| | | to a few inflammatory lesions (papules/pustules) may be |
| | | present. Post-inflammatory hyperpigmentation is allowable. |
| 2 | Mild | Easily recognizable acne; less than half the face is involved; |
| | | some comedones and some papules and pustules. |
| 3 | Moderate | More than half the face is involved; many comedones, papules |
| | | and pustules; one nodule may be present. |
| 4 | Severe | Most of the face is involved, covered with comedones, numerous |
| | | papules and pustules; up to two nodules may be present. |

### Lesion Counts

Lesions are characterized as inflammatory or non-inflammatory using the following criteria:

Inflammatory lesions:
Papule—a solid, elevated lesion less than 0.5 cm in diameter.
Pustule—an elevated lesion containing pus less than 0.5 cm in diameter.
Nodule—palpable solid lesion greater or equal to 0.5 cm in diameter; has depth, not necessarily elevated.

Non-inflammatory lesions:
Open comedones (blackhead)—non-infected plugged hair follicle with dilated/open orifice; black in color.
Closed comedones (whitehead)—non-infected plugged hair follicle; small (microscopic) opening at skin surface.

Facial lesion counts are made for the forehead, left and right cheeks, nose, and chin. Lesion counts are done when specified in Table 13. Total inflammatory lesions (pustules, papules, and nodules) and non-inflammatory lesions (open and closed comedones) are counted and recorded separately.

### Subject Satisfaction Questionnaire

A subject satisfaction questionnaire is used to collect subject opinions via categorial determinants on various aspects relating to the utility of the study drug. Subjects are asked to assess their acne and how acne impacts their appearance at the Baseline.

### Safety Assessments

The safety assessments in this study are standard safety measures used in clinical studies, including physical examinations, the monitoring of vital signs, TEAEs (volunteered, observed, and elicited by general questioning in a non-suggestive manner), and local skin tolerability assessments.

### Local Skin Tolerability Assessments

Local skin tolerability (face only) is assessed based on subject-rated itching and stinging/burning, and assessments of dryness, scaling, erythema and hyperpigmentation when specified in Table 13. Dryness, scaling, erythema and hyperpigmentation are assessed by the same evaluator throughout the study whenever possible.

Adverse Event Definitions

An “Adverse Event” (AE) is any unfavorable or unintended sign, symptom, or disease that appears or worsens in a subject after the subject signs (or a subject's parent or legal guardian in the case of subjects <18 years of age or according to state law) the ICF/Assent Form for a clinical study. Treatment-Emergent AEs (TEAEs) are defined as events that emerge during treatment having been absent pre-treatment or worsen relative to the pre-treatment state. Examples of what is considered an AE include any of the following:
A new illness
An exacerbation of a sign or symptom of an underlying condition or of a concomitant illness unrelated to participation in the clinical study or an effect of the study drug or comparator drug

Serious Adverse Events (SAE)

Treatment-emergent SAEs are recorded at each visit throughout the study. Any SAEs occurring between (and including) Screening and Baseline visits is recorded, as long as treatment has not been initiated.

A SAE is any AE that:
Results in death;
Is life-threatening; (Note: The term “life-threatening” refers to any AE that, as it occurs, puts the subject at immediate risk of death. It does not refer to an AE that hypothetically might have caused death if it were more severe.)
Results in hospitalization or prolongation of current hospitalization (not including hospitalization for a pre-existing condition that has not increased in severity or frequency from the subject's underlying medical condition prior to entry into the study);
Results in persistent or significant disability/incapacity;
Is a congenital anomaly/birth defect in the offspring of a subject; or
Is another serious (important medical event) event.

Severity of the TEAE refers to the extent to which an AE affects the subject's daily activities and differs from “Serious”, which is a regulatory classification. Severity will be categorized according to the following criteria:
Mild: The symptom has a negligible effect or no impairing effect on the subject's normal function;
Moderate: The symptom impairs the subject's normal function to some extent;
Severe: The symptom has an obvious, significantly impairing effect on the subject's normal function.

### Example 21

Formulations with Alternative Waxes and Wax Ratios

Formulations containing an active agent comprising a combination of minocycline HCl and adapalene (MCH+ADP) with different waxes and different wax ratios were prepared by a holding process.

Formulations with Caranuba wax (200519N) or Glycerol behenate (200526D) instead of HCO were prepared (Table 34A, standard deviation shown below average in each cell). The formulations generated foams of excellent quality and remained shakable at 5′C and 25° C. for 30 days. Formulations with Glycerol behenate were more shakable than Caranuba wax as they remained shakable at 40′C for 30 days as well. However, phase separation was observed for both formulations after storage at 40′C for 30 days. Without being bound by any theory it may be that if the total amount of wax is increased and or the amounts and combinations/ratios of wax are adjusted it may prevent phase separation.

A formulation with Candelilla wax (2%) instead of HCO was prepared (200614N, Table 34A) and remained shakable at 5° C. and 25° C. for 30 days. After storage at 40° C. for 30 days, the product became non-shakable. Shakability may be altered by adjustment of the ratio of Candelilla wax to beeswax to oils in the formulation. See for example Table 33F below.

A formulation with HCO and candelilla wax instead of beeswax was prepared (200621N, Table 34B) at a ratio of 1.2:2 respectively. The formulation generated a foam of excellent quality and remained shakable at 5° C. and 25° C. for 30 days. After storage at 40° C. for 30 days, the product was moderately shakable and had TM4 value in a range of 68-71.4° C. at all tested conditions. Thus, beeswax may be replaceable with candelilla wax and/or HCO to provide comparable shakability results to beeswax (compare to 190505S Table 34C). Reducing wax content and/or adjusting the ratio between waxes, e.g., HCO and beeswax, or HCO and Candelilla, or beeswax and candelilla may serve to further alter shakability.

Formulations with increasing amounts of beeswax (0.4%-3%) and constant amount of HCO (1.2%) (200303D, 200308R, 200303R, 190505S, 200308N; table 34C) all resulted in foams of excellent quality after 1-month storage at 5′C and 25° C. (Table 34D). Following 30 days of storage, all formulations remained shakable at 25° C. At 40° C., formulations with beeswax amounts in the range 0.4-2% became moderately shakable, while the formulation with 3% beeswax became non-shakable. All formulations exhibited a TM4 value in the range of 67.9-71.6° C. at all tested conditions.

A formulation (200303D; Table 34C) with 0.4% beeswax and 1.2% HCO resulted in phase separation when stored at 40° C. at day 30 as observed for Formulation 191117N (Table 20b) containing even lower amount of beeswax (1.2% HCO and 0.1% beeswax). This demonstrated that a 0.4% was below the minimal amount of beeswax required to form a sufficient matrix in the composition that holds the active agent(s) homogenously dispersed within the formulation, even in the presence of 1.2% HCO. Without being bound by theory by increasing one or more other waxes and or adjusting their ratios it may prevent phase separation.

TABLE 34A

| Study | 6.3 | 6.4 | 6.5 |
|---|---|---|---|
| Formulation # | Carnauba wax | Candelilla wax | Glycerol behenate |
| | instead of HCO | instead of HCO | instead of HCO |
| | (200519N) | (200614N) | (200526N) |
| API | MCH + ADP | MCH + ADP | MCH + ADP |
| Process | Holding | Holding | Holding |
| Component | | | |
| Soybean oil | 50 | 50 | 50 |
| coconut oil | 23.6 | 23.6 | 23.6 |
| Light mineral oil* | 2.5 | 2.5 | 2.5 |
| Stearic acid | 3 | 3 | 3 |
| Docosanol | 1.1 | 1.1 | 1.1 |
| Carnauba wax | 2 | 0 | 0 |
| Candelilla wax | 0 | 2 | |
| Glycerol behenate | 0 | 0 | 2 |
| White wax (beeswax) | 2 | 2 | 2 |
| Stearyl alcohol | 1.5 | 1.5 | 1.5 |
| Cetostearyl alcohol | 3.5 | 3.5 | 3.5 |
| Myristyl alcohol | 2.5 | 2.5 | 2.5 |
| Cyclomethicone | 5 | 5 | 5 |
| Minocycline HCI ** | 3 | 3 | 3 |
| Adapalene | 0.3 | 0.3 | 0.3 |
| TOTAL | 100 | 100 | 100 |
| Foam Quality | E | E | E |

| | Temperature/ | | | | | | | | | |
|---|---|---|---|---|---|---|---|---|---|---|
| Test | Time | T0 | T15 | T30 | T0 | T15 | T30 | T0 | T15 | T30 |
| Shakability | 5° C. | S | | S | S | | S | S | | S |
| | 25° C. | S | | S | S | | S | S | | S |
| | 40° C. | S | | NA | S | | N | | | S |
| Rheology G′ Pa | 5° C. | NA | | 219.5 | NA | NA | | NA | NA | 51.6 |
| | | | | 45.8 | | | | | | 10.3 |
| | 25° C. | 24.9 | NA | 32.6 | 49.7 | NA | | 61.1 | NA | 140.8 |
| | | 21.9 | | 3.3 | 4.8 | | | 4.1 | | 39.5 |
| | 40° C. | 25775.7 | NA | NA # | 542577.0 | NA | | 6617.2. | NA | NA # |
| | | 4117.7 | | | NA** | | | 4542.9 | | |
| Rheology | 5° C. | NA | NA | 26.4 | NA | NA | 27.1 | NA | NA | 26.4 |
| Flow Point, ° C. | | | | 0.1 | | | 0.1 | | | 0.2 |
| | 25° C. | 25.3 | NA | 26.1 | 26.4 | NA | 26.5 | 26.4 | NA | 28.2 |
| | | NA* | | 0.4 | 0.4 | | 0.1 | 0.1 | | 0.7 |
| | 40° C. | 35.1 | NA | NA # | 29.7 | NA | 34.8 | 39.8 | NA | NA # |
| | | 1.2 | | | 1.3 | | 1.4 | 0.1 | | |
| Rheology | 5° C. | NA | NA | 276.5 | NA | NA | 360.6 | NA | NA | 201.0 |
| Viscosity, cP | | | | NA** | | | NA** | | | NA** |
| | 25° C. | 184.5 | NA | 200.5 | 195.5 | NA | 208.9 | 163.4 | NA | 325.9 |
| | | NA** | | NA** | NA | | NA | NA** | | NA** |
| | 40° C. | NA | NA | NA # | NA | NA | NA | NA | NA | NA # |
| DSC TM2 | 5° C. | NA | NA | 28.3 | NA | NA | 27.4 | NA | NA | 30.2 |
| | | | | 0.2 | | | 0.0 | | | 0.2 |
| | 25° C. | 28.0 | NA | 27.7 | 27.1 | NA | 27.5 | 28.8 | NA | 29.4 |
| | | 0.0 | | 0.1 | 0.1 | | 0.1 | 0.2 | | 0.4 |
| | 40° C. | 27.5 | NA | NA # | 27.8 | NA | 27.2 | 28.3 | NA | NA # |
| | | 0.1 | | | 0.2 | | 0.1 | 0.2 | | |
| DSC TM2.1 | 5° C. | NA | NA | | NA | NA | | NA | NA | |
| | 25° C. | | NA | | | NA | | | NA | |
| | 40° C. | | NA | NA # | | NA | | | NA | NA # |
| DSC TM3.1 | 5° C. | NA | NA | | NA | NA | 38.4 | NA | NA | 39.9 |
| | | | | | | | 0.1 | | | 0.5 |
| | 25° C. | | NA | 38.5 | 38.7 | NA | 39.0 | 38.8 | NA | 39.8 |
| | | | | 0.2 | 0.4 | | 0.4 | 0.2 | | 0.4 |
| | 40° C. | 38.2 | NA | NA # | 36.2 | NA | 36.2 | 38.0 | NA | NA # |
| | | 1.7 | | | 0.1 | | 0.4 | 0.2 | | |
| DSC TM3 | 5° C. | NA | NA | 47.3 | NA | NA | | NA | NA | |
| | | | | 0.3 | | | | | | |
| | 25° C. | 46.0 | NA | 46.8 | | NA | | | NA | 44.2 |
| | | 0.7 | | 0.2 | | | | | | 0.4 |
| | 40° C. | 47.8 | NA | NA # | 44.7 | NA | 45.7 | 43.6 | NA | NA # |
| | | 0.5 | | | 0.1 | | 0.2 | 0.1 | | |
| DSC TM4.2 | 5° C. | NA | NA | 54.4 | NA | NA | 52.0 | NA | NA | 50.4 |
| | | | | 0.5 | | | 0.1 | | | 0.2 |
| | 25° C. | 53.6 | NA | 54.1 | 51.8 | NA | | 50.4 | NA | 50.9 |
| | | 0.1 | | 0.3 | 0.1 | | | 0.1 | | 0.2 |
| | 40° C. | 54.2 | NA | NA # | 51.7 | NA | 51.8 | 50.6 | NA | NA # |
| | | 0.6 | | | 0.2 | | 0.1 | 0.2 | | |
| DSC TM4.1 | 5° C. | NA | NA | | NA | NA | 63.1 | NA | NA | 55.9 |
| | | | | | | | 0.2 | | | 0.5 |
| | 25° C. | 60.7 | NA | 59.0 | 62.6 | NA | 63.1 | 55.8 | NA | 56.1 |
| | | 2.4 | | 0.2 | 0.3 | | 0.5 | 0.1 | | 0.2 |
| | 40° C. | | NA | NA # | 62.9 | NA | 62.2 | 55.9 | NA | NA # |
| | | | | | 0.5 | | 0.1 | 0.4 | | |
| DSC TM4 | 5° C. | NA | NA | 72.6 | NA | NA | | NA | NA | |
| | | | | 0.2 | | | | | | |
| | 25° C. | 72.1 | NA | 72.4 | | NA | | | NA | |
| | | 0.1 | | 0.0 | | | | | | |
| | 40° C. | 72.6 | NA | NA # | | NA | | | NA | NA # |
| | | 0.1 | | | | | | | | |

# Phase separation
*one or both replicates flow point lower than 25° C.
**only one measurement made

TABLE 34B

| | |
|---|---|
| Study | 6.7 |
| Formulation # | Candelilla wax instead |
| | of beeswax (200621N) |
| API | MCH + ADP |
| Process | Holding at 54° C. |
| Component | |
| Soybean oil | 50 |
| coconut oil | 23.6 |
| Light mineral oil | 3.3 |
| Stearic acid | 3 |
| Docosanol | 1.1 |
| Hydrogenated castor oil | 1.2 |
| Candelilla wax | 2 |
| Stearyl alcohol | 1.5 |
| Cetostearyl alcohol | 3.5 |
| Myristyl alcohol | 2.5 |
| Cyclomethicone | 5 |
| Minocycline HCl | 3 |
| Adapalene | 0.3 |
| TOTAL | 100 |
| Foam Quality | E |

| | Temperature/ | | | | |
|---|---|---|---|---|---|
| Test | Time | | T0 | T15 | T30 |
| Shakability | 15° | C. | S | | 5 |
| | 25° | C. | S | | S |
| | 40° | C. | S | | M |
| Rheology G′ Pa | 5° | C. | NA | NA | |
| | 25° | C. | | NA | |
| | 40° | C. | 50658.6 | NA | 10890.2 |
| | | | 14950.5 | | 204.5 |
| Rheology | 5° | C. | NA | NA | 27.3 |
| | | | | | 0.1 |
| Flow Point, ° C. | 25° | C. | 28.4 | NA | 36.8 |
| | | | 0.2 | | 0.2 |
| | 40° | C. | 36.7 | NA | 55.7 |
| | | | NA** | | 0.6 |
| Rheology Viscosity, cP | 5° | C. | NA | NA | 387.5 |
| | | | | | NA |
| | 25° | C. | 366.1 | NA | 366.1 |
| | | | NA** | | NA |
| | 40° | C. | NA | NA | 603.4 |
| | | | | | NA |
| DSC TM2 | 15° | C. | NA | NA | 28.3 |
| | | | | | 0.1 |
| | 25° | C. | 28.1 | NA | 28.0 |
| | | | 0.1 | | 0.0 |
| | 40° | C. | 27.4 | NA | 27.5 |
| | | | 0.2 | | 0.1 |
| DSC TM2.1 | 5° | C. | NA | NA | |
| | 25° | C. | | NA | |
| | 40° | C. | 35.7 | NA | |
| | | | 0.2 | | |
| DSC TM3.1 | 5° | C. | NA | NA | 38.7 |
| | | | | | 0.6 |
| | 25° | C. | 38.8 | NA | 39.6 |
| | | | 0.0 | | 0.7 |
| | 40° | C. | 39.8 | NA | 38.5 |
| | | | 0.2 | | 0.1 |
| DSC TM3 | 5° | C. | NA | NA | 43.1 |
| | | | | | 10.2 |
| | 25° | C. | | NA | |
| | 40° | C. | 48.8 | NA | |
| | | | 0.2 | | |
| DSC TM4.2 | 5° | C. | NA | NA | |
| | 25° | C. | | NA | |
| | 40° | C. | | NA | 53.3 |
| | | | | | 0.3 |
| DSC TM4.1 | 5° | C. | NA | NA | 57.7 |
| | | | | | 10.1 |
| | 25° | C. | 58.7 | NA | 58.1 |
| | | | 0.4 | | 0.7 |
| | 40° | C. | 58.3 | NA | 60.1 |
| | | | 0.1 | | 0.3 |
| DSC TM4 | 15° | C. | NA | NA | 69.2 |
| | | | | | 0.3 |
| | 25° | C. | 71.2 | NA | 69.6 |
| | | | 1.3 | | 0.1 |
| | 140° | C. | 71.4 | NA | 68.0 |
| | | | 0.0 | | 10.0 |

**only one measurement made

TABLE 34C

| Study | 7.9 | 7.10 | 7.8 | 3.9/7.3 | 7.11 |
|---|---|---|---|---|---|
| Formulation # | 200303D | 200308R | 200303R | 190505S | 200308N |
| HCO amount | 1.2% | 1.2% | 1.2% | 1.2% | 1.2% |
| API | MCH + | MCH + | MCH + | MCH + | MCH + |
| | ADP | ADP | ADP | ADP | ADP |
| Process | Holding | Holding | Holding at | Holding | Holding |
| | at 54 ° C. | at 54 ° C. | 54 ° C. | at 54 ° C. | at 54 ° C. |
| Component | | | | | |
| Soybean oil | 50 | 50 | 50 | 50 | 50 |
| coconut oil | 23.6 | 23.6 | 23.6 | 23.6 | 23.6 |
| Light mineral oil | 4.9 | 4.5 | 4.1 | 3.3 | 2.3 |
| Stearic acid | 3 | 3 | 3 | 3 | 3 |
| Docosanol | 1.1 | 1.1 | 1.1 | 1.1 | 1.1 |
| Hydrogenated castor | 1.2 | 1.2 | 1.2 | 1.2 | 1.2 |
| oil | | | | | |
| White wax (beeswax) | 0.4 | 0.8 | 1.2 | 2 | 3 |
| Stearyl alcohol | 1.5 | 1.5 | 1.5 | 1.5 | 1.5 |
| Cetostearyl alcohol | 3.5 | 3.5 | 3.5 | 3.5 | 3.5 |
| Myristyl alcohol | 2.5 | 2.5 | 2.5 | 2.5 | 2.5 |
| Cyclomethicone | 5 | 5 | 5 | 5 | 5 |
| Minocycline HCI | 3 | 3 | 3 | 3 | 3 |
| Adapalene | 0.3 | 0.3 | 0.3 | 0.3 | 0.3 |
| TOTAL | 100 | 100 | 100 | 100 | 100 |

| | Temper- | | | | | | | | |
|---|---|---|---|---|---|---|---|---|---|
| | ature/ | | | | | | | | |
| Test | Time | T0 | T15! | T30 | T0 | T15 | T30 | T0 | T15! |
| Shakability | 5° C. | S | | S | S | S | S | S | |
| | 25° C. | S | | S | S | S | S | S | |
| | 40° C. | S | | M | S | M | M | S | |
| Rheology | 5° C. | | | 76 | NA | 24.0 | 79.5 | Na | |
| G′ Pa | | | | 24.7 | | 5.7 | 7.8 | | |
| | 25° C. | 13 | | 281 | 14.0 | 127.5 | 262.5 | 22.0 | |
| | | NA | | 29.7 | 2.8 | 64.3 | 72.8 | 2.1 | |
| | 40° C. | 1600 | | 31 | 4145.0 | 4553.0 | 4469.0 | 4044 | |
| | | 233.3 | | NA | 745.3 | 3359.2 | 862.7 | 1510 | |
| | | | | 2108810.3* | | | | | |
| Rheology | 5° C. | NA | | 26.4 | NA | 25.6 | 26.3 | Na | |
| Flow | | | | 0.92 | | 0.0 | 0.1 | | |
| Point, ° C. | 25° C. | 26.0 | | 35.7 | NA* | 32.3 | 35.3 | 24.5 | |
| | | NA | | 0.9 | | 0.4 | 0.0 | 1.3 | |
| | 40° C. | 30.3 | | NA | 31.4 | 57.9 | 60.3 | 31.1 | |
| | | 0.7 | | | 0.3 | 0.4 | 0.2 | 2.47 | |
| Rheology | 5° C. | NA | | 227.3 | NA | 221.3** | 232.5** | NA | |
| Viscosity, | | | | NA | | | | | |
| cP | 25° C. | NA | | 303.4 | 202.4** | 263.8** | 288.0** | 172.2 | |
| | 40° C. | NA | | NA | NA | NA | NA | NA | |
| DSC | 5° C. | NA | | 26.9 | NA | 27.0 | 27.0 | | |
| TM2 | | | | 0.1 | | 0.1 | 0.1 | | |
| | 25° C. | 27.5 | | 27.5 | 26.8 | 27.5 | 27.7 | 27.7 | |
| | | 0.0 | | 0.1 | 0.0 | 0.1 | 0.4 | 0.1 | |
| | 40° C. | 26.9 | | 27.6 | 26.6 | 27.3 | | 27.1 | |
| | | 0.1 | | 0.1$ | 0.0 | 0.1 | | 0.2 | |
| DSC | 5° C. | | | | NA | | | | |
| TM2.1 | 25° C. | | | | | | | | |
| | 40° C. | | | | | | 31.4 | | |
| | | | | | | | 0.1 | | |
| DSC | 5° C. | | | | | | | | |
| TM3.1 | 25° C. | | | | | | | | |
| | 40° C. | | | | | | | | |
| DSC | 5° C. | | | | NA | 37.1 | 38.3 | NA | |
| TM3 | | | | | | 0.0 | 0.1 | | |
| | 25° C. | | | | 38.5 | 38.6 | 37.8 | 41.4 | |
| | | | | | 0.3 | 0.2 | 0.1 | 0.1 | |
| | 40° C. | | | | 37.7 | 38.2 | 38.6 | 40.2 | |
| | | | | | 0.1 | 0.1 | 0.1 | 0.3 | |
| DSC | 5° C. | | | | NA | | 47.5 | | |
| TM4.2 | | | | | | | 0.6 | | |
| | 25° C. | | | | 47.4 | | | | |
| | | | | | 0.0 | | | | |
| | 40° C. | | | | 47.7 | | | | |
| | | | | | 0.1 | | | | |
| DSC | 5° C. | | | | NA | 54.9 | 55.5 | | |
| TM4.1 | | | | | | 1.0 | 0.4 | | |
| | 25° C. | | | | 55.1 | | | | |
| | | | | | 0.4 | | | | |
| | 40° C. | | | | 55.5 | 54.5 | 56.1 | | |
| | | | | | 0.6 | 0.1 | 0.6 | | |
| DSC | 5° C. | NA | | 69.6 | NA | 70.6 | 70.8 | NA | |
| TM4 | | | | 0.1 | | 0.6 | 0.4 | | |
| | 25° C. | 69.7 | | 69.5 | 70.9 | 70.8 | 71.6 | 70.0 | |
| | | 0.1 | | 0.3 | 0.6 | 0.0 | 0.6 | 0.4 | |
| | 40° C. | 69.8 | | 69.2 | 0.4 | 70.2 | 70.2 | 69.9 | |
| | | 0.6 | | 0.0$$ | 70.7 | 0.1 | 0.1 | 0.3 | |

| | Temper- | | | | | | | |
|---|---|---|---|---|---|---|---|---|
| | ature/ | | | | | | | |
| Test | Time | T30 | T0 | T15 | T30 | T0 | T15 | T30 |
| Shakability | 5° C. | S | S | S | | S | S | S |
| | 25° C. | S | S | S | S | S | S | S |
| | 40° C. | M | S | M | M | S | | N |
| Rheology | 5° C. | 43 | NA | 122.1 | 128.9 | NA | 62.3 | 144.9 |
| G′ Pa | | 24.0 | | 11.1 | 27.3 | | 25.5 | 31.7 |
| | 25° C. | 215 | 58.9 | 346.4 | 434.2 | 94.0 | 1598.2 | 762.5 |
| | | 11.3 | 1.4 | 29.5 | 10.9 | 5.2 | 60.3 | 144.5 |
| | 40° C. | 4173 | 12372.5 | 7884.6 | 5933.6 | 44641.4 | 9264.5 | 7944.2 |
| | | 1070.2 | 482.2 | 1891.3 | 584.7 | 4757.5 | 897.2 | 68.8 |
| Rheology | 5° C. | 26.4 | NA | 26.4 | 26.7 | NA | 26.2 | 26.6 |
| Flow | | 0.2 | | 0.8 | 1.1 | | 0.4 | 0.9 |
| Point, ° C. | 25° C. | 35.4 | 24.9 | 34.6 | 36.3 | 26.0 | 36.8 | 38.2 |
| | | 0.1 | 0.5 | 0.5 | 0.2 | 0.1 | 0.2 | 0.5 |
| | 40° C. | 55.6 | 32.2 | 53.65 | 54.85 | 33.4 | 53.0 | 54.3 |
| | | 0.9 | 0.0 | 0.5 | 1.3 | 0.4 | 0.4 | 0.1 |
| Rheology | 5° C. | 244.4 | NA | 244.5** | 291.6** | NA | 326.4** | 379.1** |
| Viscosity, | | | | | | | | |
| cP | 25° C. | 265.4 | 251.1** | NA | 368.9** | 266.3** | 455.6** | 455.7** |
| | 40° C. | NA | NA | NA | NA | NA | NA | NA |
| DSC | 5° C. | 27.0 | NA | 27.1 | 26.8 | NA | 27.3 | 26.9 |
| TM2 | | 0.0 | | 0.1 | 0.1 | | 0.1 | 0.1 |
| | 25° C. | 27.3 | 26.9 | 27.1 | 27.1 | 27.4 | 27.3 | 27.0 |
| | | 0.1 | 0.1 | 0.1 | 0.1 | 0.1 | 0.1 | 0.0 |
| | 40° C. | 30.0 | 26.5 | | | 26.9 | 27.1*** | |
| | | NA | 0.1 | | | 0.1 | | |
| DSC | 5° C. | | NA | | | | | |
| TM2.1 | 25° C. | | | | | | | |
| | 40° C. | | 31.9 | 29.2 | 30.3 | | | |
| | | | 0.3 | 2.0 | 0.3 | | | |
| DSC | 5° C. | | NA | | | | | |
| TM3.1 | 25° C. | | | | | | 37.1*** | 38.4 |
| | | | | | | | | 0.1 |
| | 40° C. | | 39.9 | 38.3 | 38.8 | | | 36.4 |
| | | | 0.3 | 0.1 | 0.3 | | | 0.6 |
| DSC | 5° C. | 41.3 | NA | 42.4 | 42.1 | NA | 44.8 | 44.9 |
| TM3 | | 0.1 | | 0.1 | 0.1 | | 0.1 | 0.2 |
| | 25° C. | 40.9 | 42.1 | 42.3 | 41.9 | 44.7 | 45.0 | 45.0 |
| | | 0.0 | 0.2 | 0.7 | 0.2 | 0.2 | 0.0 | 0.2 |
| | 40° C. | 40.6 | 43.5 | 43.8 | 44.1 | 44.8 | 45.6 | 45.7 |
| | | 0.0 | 0.1 | 0.1 | 0.2 | 0.0 | 0.3 | 0.2 |
| DSC | 5° C. | | NA | | | | | |
| TM4.2 | | | | | | | | |
| | 25° C. | | | | | | | |
| | 40° C. | 55.4 | | 53.7 | 56.1 | | | |
| | | 0.5 | | 0.1 | 0.5 | | | |
| DSC | 5° C. | | NA | | | | | |
| TM4.1 | | | | | | | | |
| | 25° C. | | | | | | | |
| | 40° C. | | | | | | | |
| DSC | 5° C. | 69.9 | NA | 68.5 | 68.6 | NA | 68.8 | 68.8 |
| TM4 | | 0.4 | | 0.4 | 0.2 | | 0.8 | 0.1 |
| | 25° C. | 70.7 | 69.3 | 69.2 | 68.8 | 68.8 | 69.2 | 68.4 |
| | | 0.9 | 0.9 | 0.2 | 0.5 | 0.7 | 0.0 | 0.1 |
| | 40° C. | 69.3 | 69.4 | 68.4 | 68.6 | 69.0 | 67.9 | 68.0 |
| | | 0.7 | 0.3 | 0.2 | 0.4 | 0.6 | 0.2 | 0.0 |

*Separated into two phases. Results for lower phase are presented below the results for upper phase.
$Separated into two phases. Results presented for upper phase only.
$$ Separated into two phases. Results presented for lower phase only.
*one or both replicates flow point lower than 25° C.
**Only one replicate was measured//only one measurement made
****only one measurement presented this transition
!Not measured at 15 days for these samples.

TABLE 34D

| | | Formulation # | | | |
|---|---|---|---|---|---|
| | | 200303R | 200303D | 200308R | 200308D |
| | | study 7.8 | Study 7.9 | Study 7.10 | Study 7.11 |
| T0 5° C. | Foam | E | E | E | E |
| | quality | | | | |
| | Collapse | 150 | 130 | >180 | >180 |
| | time | | | | |
| | Time to | 150 | 120 | 180 | >180 |
| | FG | | | | |
| T0 25° C. | Foam | E | E | E | E |
| | quality | | | | |
| | Collapse | 150 | 130 | >180 | >180 |
| | time | | | | |
| | Time to | 150 | 120 | 180 | >180 |
| | FG | | | | |
| 2 W 5° C. | Foam | NA | NA | E | E |
| | quality | | | | |
| | Collapse | NA | NA | >180 | >180 |
| | time | | | | |
| | Time to | NA | NA | 180 | >180 |
| | FG | | | | |
| 2 W 25° C. | Foam | NA | NA | E | E |
| | quality | | | | |
| | Collapse | NA | NA | >180 | >180 |
| | time | | | | |
| | Time to | NA | NA | 180 | >180 |
| | FG | | | | |
| 1 M 5° C. | Foam | E | E | E | E |
| | quality | | | | |
| | Collapse | >180 | 180 | 150 | >180 |
| | time | | | | |
| | Time to | 150 | 135 | 120 | 150 |
| | FG | | | | |
| 1 M 25° C. | Foam | E | E | E | E |
| | quality | | | | |
| | Collapse | >180 | >180 | 105 | 180 |
| | time | | | | |
| | Time to | 150 | 180 | 90 | 150 |
| | FG | | | | |

Formulations with varying ratios of beeswax to hydrogenated castor oil (15:4 and 1:4) are prepared (Table 34E) to determine the ratio which demonstrates shakability yet avoids phase separation. The ratio of beeswax to hydrogenated castor oil is 15:4 and 1:4 for formulation 7.12 and 7.13 respectively.

TABLE 34E

| | | |
|---|---|---|
| Study | 7.12 | 7.13 |
| Formulation # | | |
| HCO amount | 0.8% | 1.6% |
| API | MCH + ADP | MCH + ADP |
| Process | Holding at 54° C. | Holding at 54° C. |
| Component | | |
| Soybean oil | 50 | 50 |
| coconut oil | 23.6 | 23.6 |
| Light mineral oil | 2.7 | 4.5 |
| Stearic acid | 3 | 3 |
| Docosanol | 1.1 | 1.1 |
| Hydrogenated castor oil | 0.8 | 1.6 |
| White wax (beeswax) | 3 | 0.4 |
| Stearyl alcohol | 1.5 | 1.5 |
| Cetostearyl alcohol | 3.5 | 3.5 |
| Myristyl alcohol | 2.5 | 2.5 |
| Cyclomethicone | 5 | 5 |
| Minocycline HCl | 3 | 3 |
| Adapalene | 0.3 | 0.3 |
| TOTAL | 100 | 100 |

Formulations containing 0.5%, or 0.8%, or 1.2%/of Candelilla wax instead of HCO, with 3% minocycline HCl, and 0.3% adapalene (MCH+ADP), are prepared in a holding process (Table 34F). In one or more embodiments, the decrease in the amount of alternate wax like candelilla wax, can provide a better shakability of the drug product e.g., following storage at 40-0.

TABLE 34F

| Formulation # | 0.5% Candelilla wax | 0.8% Candelilla wax | 1.2% Candelilla wax |
|---|---|---|---|
| | instead of HCO | instead of HCO | instead of HCO |
| API | MCH + ADP | MCH + ADP | MCH + ADP |
| Process | Holding | Holding | Holding |
| Component | | | |
| Soybean oil | 50 | 50 | 50 |
| coconut oil | 23.6 | 23.6 | 23.6 |
| Light mineral oil | 4 | 3.7 | 3.3 |
| Stearic acid | 3 | 3 | 3 |
| Docosanol | 1.1 | 1.1 | 1.1 |
| Candelilla wax | 0.5 | 0.8 | 1.2 |
| White wax (beeswax) | 2 | 2 | 2 |
| Stearyl alcohol | 1.5 | 1.5 | 1.5 |
| Cetostearyl alcohol | 3.5 | 3.5 | 3.5 |
| Myristyl alcohol | 2.5 | 2.5 | 2.5 |
| Cyclomethicone | 5 | 5 | 5 |
| Minocycline HCl | 3 | 3 | 3 |
| Adapalene | 0.3 | 0.3 | 0.3 |
| TOTAL | 100 | 100 | 100 |

### Example 22 Alternative Emollients and Emollient Ratios

Formulations containing an active agent comprising a combination of minocycline HCl and adapalene (MCH+ADP) with different emollients and different emollient ratios were prepared by a holding process.

Formulations comprising 50% corn oil (202505D) or 50% safflower oil (202505R) instead of soybean oil were prepared (Table 35A). Both formulations generated foams of excellent quality. Following 30 days of storage at 40C, the formulation comprising 50% corn oil was moderately shakable, while the formulation comprising 50% safflower oil became non-shakable. Both formulations exhibited a TM4 value in the range 70.0-71.5° C. at all tested conditions. Safflower oil is very rich in polyunsaturated fatty acids and having about over 80%/of linoleic acid. Corn oil and soybean oil in comparison may have about 58-62% and 55% linoleic acid content. Without being bound by theory, it may be that safflower oil interacts more strongly than corn oil with the waxes and/or fatty acids and/or fatty alcohols in the tested formulations, resulting in a stronger network and which in turn can become non-shakable after 30 days of storage at 40° C. Reducing the wax content and/or changing the ratios (see e.g., Table 350) may result in an improved shakability.

TABLE 35A

| | | |
|---|---|---|
| Study | 9.4 | 9.5 |
| Formulation # | Corn oil instead of | Safflower oil instead of |
| | soybean oil | soybean oil |
| | (202505D) | (202505R) |
| HCO amount | 1.2% | 1.2% |
| API | MCH + ADP | MCH + ADP |
| Process | Holding at 54° C. | Holding at 54° C. |
| Component | | |
| Corn oil | 50 | 0 |
| Safflower | 0 | 50 |
| Soybean oil | 0 | 0 |
| coconut oil | 23.6 | 23.6 |
| Light mineral oil* | 3.3 | 3.3 |
| Stearic acid | 3 | 3 |
| Docosanol | 1.1 | 1.1 |
| Hydrogenated castor oil | 1.2 | 1.2 |
| White wax (beeswax) | 2 | 2 |
| Stearyl alcohol | 1.5 | 1.5 |
| Cetostearyl alcohol | 3.5 | 3.5 |
| Myristyl alcohol | 2.5 | 2.5 |
| Cyclomethicone | 5 | 5 |
| Minocycline HCl | 3 | 3 |
| Adapalene | 0.3 | 0.3 |
| TOTAL | 100 | 100 |
| Foam Quality | E | E |

| | Temperature/ | | | | | |
|---|---|---|---|---|---|---|
| Test | Time | | T0 | T30 | T0 | T30 |
| Shakability | 5° | C. | S | S | S | S |
| | 25° | C. | S | S | S | S |
| | 40° | C. | S | M | S | N |
| Rheology | 5° | C. | NA | 129.2* | NA | 39.5 |
| G′ Pa | | | | | | 10.6 |
| | 25° | C. | 51.7 | 109.4* | 55.5 | 238.0 |
| | | | 5.2 | | 2.1 | 33.9 |
| | 40° | C. | 22855.7 | 1843.1* | 18336.0 | 12081.5 |
| | | | 1192.3 | | 2698.3 | 2505.3 |
| Rheology | 5° | C. | NA | 26.4 | NA | 25.4 |
| Flow | | | | 0.7 | | 0.3 |
| Point, ° C. | 25° | C. | 26.3 | 29.7* | 25.4 | 32.7 |
| | | | 0.1 | | 0.7 | 0.4 |
| | 40° | C. | 33.0 | 51.2* | 32.6 | 57.4 |
| | | | 0.3 | | 0.2 | 0.5 |
| Rheology | 5° | C. | NA | 305.8* | NA | 260.0* |
| Viscosity, | 25° | C. | 231.9* | 335.7* | 257.7* | 347.0* |
| cP | 40° | C. | | | NA | NA |
| DSC TM2 | 5° | C. | NA | 27.7 | NA | 27.7 |
| | | | | 0.0 | | 0.2 |
| | 25° | C. | 28.0 | 28.0 | 27.9 | 27.6 |
| | | | 0.1 | 0.0 | 0.3 | 0.4 |
| | 40° | C. | 26.9 | 27.8 | 27.5 | 27.4 |
| | | | 0.1 | 0.1 | 0.4 | 0.2 |
| DSC TM2.1 | 5° | C. | NA | NA | NA | |
| | 25° | C. | | | | |
| | 40° | C. | | 32.6 | | |
| | | | | 0.2 | | |
| DSC TM3.1 | 5° | C. | NA | | NA | |
| | 25° | C. | | | | 38.5 |
| | | | | | | 0.2 |
| | 40° | C. | 40.1 | 38.7 | 40.1 | 39.8 |
| | | | 0.1 | 0.9 | 0.7 | 0.1 |
| DSC TM3 | 5° | C. | NA | 44.1 | NA | 43.9 |
| | | | | 0.0 | | 0.3 |
| | 25° | C. | 44.4 | 44.1 | 43.0 | 43.4 |
| | | | 0.8 | 0.0 | 0.5 | 0.0 |
| | 40° | C. | 44.1 | 45.3 | 44.1 | 45.4 |
| | | | 0.2 | 0.2 | 0.5 | 0.1 |
| DSC TM4.2 | 5° | C. | NA | | NA | |
| | 25° | C. | | | | |
| | 40° | C. | 56.1 | | | |
| | | | 0.5 | | | |
| DSC TM4.1 | 5° | C. | NA | | NA | 56.9 |
| | | | | | | 0.2 |
| | 25° | C. | | | 56.6 | 57.4 |
| | | | | | 0.1 | 0.0 |
| | 40° | C. | 61.3 | 59.6 | 57.2 | 54.6 |
| | | | 3.0 | 1.1 | 1.0 | 1.8 |
| DSC TM4 | 5° | C. | NA | 70.7 | NA | 70.6 |
| | | | | 0.4 | | 0.5 |
| | 25° | C. | 70.5 | 70.7 | 70.7 | 71.5 |
| | | | 0.8 | 0.6 | 0.4 | 0.6 |
| | 40° | C. | 70.4 | 70.0 | 71.0 | 71.0 |
| | | | 0.2 | 0.4 | 0.6 | 0.1 |

*only one measurement made

A formulation comprising 70% coconut oil and 3.6%/soybean oil is prepared as set out in Table 35B.

TABLE 35B

| | |
|---|---|
| Study | 8.4 |
| Formulation # | 70% Coconut |
| | oil; 3.6% Soybean oil |
| HCO amount | 1.2% |
| API | MCH + ADP |
| Process | Holding at 54° C. |
| Component | |
| Soybean oil | 3.6 |
| coconut oil | 70 |
| Light mineral oil* | 3.3 |
| Stearic acid | 3 |
| Docosanol | 1.1 |
| Hydrogenated castor oil | 1.2 |
| White wax (beeswax) | 2 |
| Stearyl alcohol | 1.5 |
| Cetostearyl alcohol | 3.5 |
| Myristyl alcohol | 2.5 |
| Cyclomethicone | 5 |
| Minocycline HCl** | 3 |
| Adapalene | 0.3 |
| TOTAL | 100 |

Formulations containing e.g., safflower oil instead of soybean oil and comprising a combination of minocycline HCl and adapalene (MCH+ADP) with a lower amount of wax (e.g., HCO and beeswax) are prepared by a holding process (Table 35C). The reduction in the wax may result in an improved shakability e.g., when subjected to storage at 40° C..

TABLE 35C

| Formulation # | Safflower oil | Safflower oil | Safflower oil |
|---|---|---|---|
| | with 13.5% waxes | with 11.9% waxes | with 10.5% waxes |
| HCO amount | 1.2% | 1.2% | 1.2% |
| API | MCH + ADP | MCH + ADP | MCH + ADP |
| Process | Holding at 54° C. | Holding at 54° C. | Holding at 54° C. |
| Component | | | |
| Safflower | 50 | 50 | 50 |
| Coconut oil | 23.6 | 23.6 | 23.6 |
| Light mineral oil | 4.6 | 6.2 | 7.6 |
| Stearic acid | 2.7 | 2.4 | 2.1 |
| Docosanol | 1.0 | 0.9 | 0.8 |
| Hydrogenated castor oil | 1.1 | 1.0 | 0.8 |
| White wax (beeswax) | 1.8 | 1.6 | 1.4 |
| Stearyl alcohol | 1.4 | 1.2 | 1.1 |
| Cetostearyl alcohol | 3.2 | 2.8 | 2.5 |
| Myristyl alcohol | 2.3 | 2.0 | 1.8 |
| Cyclomethicone | 5 | 5 | 5 |
| Minocycline HCl | 3 | 3 | 3 |
| Adapalene | 0.3 | 0.3 | 0.3 |
| TOTAL | 100 | 100 | 100 |

### Example 23

### Alternative Processes

Formulations containing an active agent comprising a combination of minocycline HCl and adapalene (MCH+ADP) were prepared using different processes as shown in Table 36A. The same formulation was prepared using different holding temperatures and holding periods. Formulations 4.7 and 10.5 were prepared with a holding step at 56° C. for 4 hr and 2 hr respectively. Formulation 10.6 was prepared with a holding step at 54° C. for 2 hr. Following 30 days of storage at 5° C. and 25° C. all formulations were shakable. However at 40° C. only the formulations generated by holding for 4 hr at 56° C. (formulation 4.7) or 60° C. (formulation 10.3) remained shakable, while the formulations with a shorter holding period of 2 hr either at 56° C. or 54° C. (formulations 10.5 and 10.6) became non-shakable over time. Comparison between formulation 4.7 and formulation 10.5 where the sole difference was the holding period further demonstrated that shakability was improved with a longer holding step at 56° C. (4 hr versus 2 hr) at 40° C. after 30 days and higher TM4 closer to batches with holding for 4 hr at 54° C. The lowest TM4 value range (65.5-66.2° C.) was demonstrated for the formulation held for 2 hr at 56° C., while the highest TM4 value range (71.9-72.2° C.) was demonstrated for the formulation held for 4 hr at 60° C. (shear-additional components). For the two other processes the TM4 value range was between 68.4-70.4° C. The TM4 value for 2 hr at 56C (formulation 10.6) was similar to batches with holding for 4 hr at 54° C.

Formulation 10.3 was prepared using the following process: part of the soybean oil (47.5% of total composition) and HCO were heated to 90-95° C., cooled down to 60° C. and held at 60° C. for 4 hours. In a second vessel, all other waxes and coconut oil were heated to 90-95° C. and cooled down to 60° C. This mixture was added to the soybean oil mixture and cooled down to 38° C. Minocycline HCl and cyclomethicone were added, and the mixture was homogenized for 10 minutes. The mixture was cooled down to 26° C. In a third vessel, a slurry of adapalene with the remaining soybean oil (2.5%) was prepared. The slurry was added to the rest of the bulk, and the resulting formulation was cooled down to 22° C. The formulation generated a foam of excellent quality, and remained shakable following 30 days of storage at 40° C. At all tested condition, this formulation exhibited a TM4 value in the range 71.9-72.7.

TABLE 36A

| Study | 10.3 | 4.7 | 10.5 | 10.6 |
|---|---|---|---|---|
| Formulation # | | | | |
| API | MCH + ADP | MCH + ADP | MCH + ADP | MCH + ADP |
| Process | Split waxes | Holding for 4h at | Holding for 2hr at | Holding for 2hr |
| | (200531N) | 56° C. | 56° C. | at 54° C. |
| | | (200426D) | (200421N) | (200421S) |
| Component | | | | |
| Soybean oil | 50 | 50 | 50 | 50 |
| coconut oil | 23.6 | 23.6 | 23.6 | 23.6 |
| Light mineral | 3.3 | 3.3 | 3.3 | 3.3 |
| oil* | | | | |
| Stearic acid | 3 | 3 | 3 | 3 |
| Docosanol | 1.1 | 1.1 | 1.1 | 1.1 |
| Hydrogenated | 1.2 | 1.2 | 1.2 | 1.2 |
| castor oil | | | | |
| White wax | 2 | 2 | 2 | 2 |
| (beeswax) | | | | |
| Stearyl alcohol | 1.5 | 1.5 | 1. 5 | 1.5 |
| Cetostearyl | 3.5 | 3.5 | 3.5 | 3.5 |
| alcohol | | | | |
| Myristyl alcohol | 2.5 | 2.5 | 2.5 | 2.5 |
| Cyclomethicone | 5 | 5 | 5 | 5 |
| Minocycline | 3 | 3 | 3 | 3 |
| HCI** | | | | |
| Adapalene | 0.3 | 0.3 | 0.3 | 0.3 |
| TOTAL | 100 | 100 | 100 | 100 |

| | Temper- | | | | | | | | | | | | |
|---|---|---|---|---|---|---|---|---|---|---|---|---|---|
| | ature/ | | | | | | | | | | | | |
| Test | Time | T0 | ! | T30 | T0 | ! | T30 | T0 | ! | T30 | T0 | ! | T30 |
| Shak- | 5° C. | S | | S | S | | S | S | | S | S | | S |
| ability | 25° C. | S | | S | S | | S | S | | S | S | | S |
| | 40° C. | S | | S | S | | S | S | | N | S | | N |
| Rheology | 5° C. | NA | | 424.0 | NA | | 117.7 | | NA | 702.0 | NA | NA | 130.9 |
| G′ Pa | | | | 62.2 | | | 7.7 | | | 141.4 | | | 117.6 |
| | 25° C. | 68.5 | | 507.0 | 46.3 | | 287.9 | 78.0 | NA | 3941.0 | 80.5 | NA | 1353.0 |
| | | 14.8 | | 69.3 | 2.2 | | 5.5 | 14.1 | | 256.0 | 6.4 | | 1265.7 |
| | 40° C. | 3340.5 | | 7067.0 | 8472.4 | | 10303.7 | 10998.0 | NA | 12812.0 | 8696.0 | NA | 1792.0 |
| | | 212.8 | | 516.2 | 2847.9 | | 3520.3 | 2108.6 | | 1045.1 | 441.2 | | 1861.1 |
| Rheology | 5° C. | NA | | 26.6 | NA | | 26.3** | | NA | 27.4 | NA | NA | 27.3* |
| Flow | | | | 0.1 | | | | | | 0.0 | | | |
| Point, | 25° C. | 26.9** | | 36.5 | 25.7 | | 33.4 | 26.3 | NA | 40.3 | 25.9 | NA | 36.6* |
| ° C. | | | | 0.4 | 0.2 | | 1.7 | 0.0 | | 1.0 | 0.3 | | |
| | 40° C. | 36.5 | | 56.2 | 32.5 | | 59.8 | 32.6 | NA | 59.0 | 32.2 | NA | 55.2* |
| | | 0.4 | | 0.1 | 0.6 | | 0.9 | 0.4 | | 2.8 | 0.4 | | |
| Rheology | 5° C. | NA | | 349.6* | NA | | 350.8 | | NA | 418.7* | NA | NA | 361.8* |
| Viscosity, | | | | | | | NA | | | | | | |
| cP | 25° C. | 221.0* | | 350.6* | 248.5 | | 312.9 | 319.1* | NA | 799.1* | 250.5* | NA | 774.3* |
| | | | | | NA | | NA | | | | | | |
| | 40° C. | NA | | | NA | | NA | NA | NA | NA | NA | NA | NA |
| DSC | 5° C. | NA | | 27.2 | NA | | 27.4 | | NA | 27.7 | NA | NA | 27.5 |
| TM2 | | | | 0.0 | | | 0.3 | | | 0.0 | | | 0.1 |
| | 25° C. | 27.5 | | 27.4 | 28.3 | | 27.8 | 27.7 | NA | 28.9 | 27.6 | NA | 28.3 |
| | | 0.0 | | 0.3 | 0.2 | | 0.4 | 0.1 | | 0.1 | 0.0 | | 0.4 |
| | 40° C. | 27.3 | | | 27.4 | | 27.9 | 27.2 | NA | | 27.1 | NA | 28.0 |
| | | 0.1 | | | 0.1 | | 0.3 | 0.3 | | | 0.1 | | 0.7 |
| DSC | 5° C. | NA | | | NA | | | | NA | | NA | NA | |
| TM2.1 | 25° C. | | | | | | | | NA | | | NA | |
| | 40° C. | | | 31.0 | | | | | NA | | | NA | 31.8 |
| | | | | 0.3 | | | | | | | | | 0.5 |
| DSC | 5° C. | NA | | | NA | | | | NA | | NA | NA | |
| TM3.1 | 25° C. | | | | | | | | NA | | | NA | |
| | 40° C. | | | 39.9 | | | 40.2 | | NA | 40.4 | 39.9 | NA | 40.4 |
| | | | | 0.1 | | | 0.1 | | | 0.3 | 0.4 | | 0.4 |
| DSC | 5° C. | NA | | 42.4 | NA | | 43.4 | | NA | 43.3 | NA | NA | 43.1 |
| TM3 | | | | 0.1 | | | 0.3 | | | 0.0 | | | 0.2 |
| | 25° C. | 43.7 | | 42.7 | 43.3 | | 43.8 | 44.3 | NA | 43.6 | 44.4 | NA | 43.3 |
| | | 0.1 | | 0.3 | 0.2 | | 0.3 | 0.4 | | 0.1 | 0.1 | | 0.1 |
| | 40° C. | 44.4 | | 45.3 | 43.9 | | 45.6 | 44.0 | NA | 45.0 | 43.6 | NA | 45.1 |
| | | 0.3 | | 0.0 | 0.1 | | 0.5 | 0.4 | | 0.1 | 0.1 | | 0.5 |
| DSC | 5° C. | NA | | | NA | | | | NA | | NA | NA | |
| TM4.2 | 25° C. | | | | | | | | NA | | | NA | |
| | 40° C. | | | | | | 56.1 | | NA | | | NA | |
| | | | | | | | 0.6 | | | | | | |
| DSC | 5° C. | NA | | | NA | | | | NA | | NA | NA | 59.6 |
| TM4.1 | | | | | | | | | | | | | 0.2 |
| | 25° C. | | | | | | | | NA | | 59.4 | NA | 58.4 |
| | | | | | | | | | | | 0.9 | | 0.2 |
| | 40° C. | | | | 61.3 | | 61.8 | | NA | 60.9 | 59.1 | NA | 60.7 |
| | | | | | 0.5 | | 0.0 | | | 0.3 | 0.1 | | 0.9 |
| DSC | 5° C. | NA | | 71.9 | NÅ | | 66.8 | | NA | 66.3 | NA | NA | 70.3 |
| TM4 | | | | 0.1 | | | 0.7 | | | 0.7 | | | 0.2 |
| | 25° C. | 72.6 | | 72.2 | 68.5 | | 70.0 | 66.2 | NA | 65.5 | 70.4 | NA | 70.2 |
| | | 0.3 | | 0.2 | 0.2 | | 1.5 | 0.5 | | 0.5 | 0.1 | | 0.5 |
| | 40° C. | 72.7 | | 72.4 | 68.4 | | 71.6 | 65.6 | NA | | 170.4 | NA | 70.5 |
| | | 0.1 | | 0.0 | 1.1 | | 0.4 | 0.3 | | | 0.3 | | 0.7 |

*only one measurement made
**one or both replicates flow point lower than 25° C.
!measurements not conducted at 15 day.

A formulation containing an active agent comprising a combination of minocycline HCl and adapalene (MCH+ADP) is prepared by a two-step holding process the first at 54° C. for 2 hours followed by a second at 45° C. for 2 hours (Table 36B).

TABLE 36B

| | |
|---|---|
| Study | 10.4 |
| Formulation # | |
| API | MCH + ADP |
| Process | Holding for 2 hr |
| | at 54° C. and 2 hr |
| | at 45° C. |
| Component | |
| Soybean oil | 50 |
| coconut oil | 23.6 |
| Light mineral oil* | 3.3 |
| Stearic acid | 3 |
| Docosanol | 1.1 |
| Hydrogenated castor oil | 1.2 |
| White wax (beeswax) | 2 |
| Stearyl alcohol | 1.5 |
| Cetostearyl alcohol | 3.5 |
| Myristyl alcohol | 2.5 |
| Cyclomethicone | 5 |
| Minocycline HCl** | 3 |
| Adapalene | 0.3 |
| TOTAL | 100 |

### Example 24

Oil studies in a continuous-heating cooling process.

Mixtures of different oils (soybean, coconut, isopropyl myristate, mineral oil and MCT oil) with hydrogenated castor oil (HCO) with APIs (MCH and ADP) were prepared (Table 37A) in a continuous-heating cooling process and analyzed by DSC. As can be seen in Table 37A, mixtures of HCO in isopropyl myristate, MCT oil, light mineral oil, soybean oil and coconut oil presented TM4 peak. Mixtures of HCO in soybean oil and light mineral oil showed TM4 peak at 72.3° C. and 80.3° C., respectively. Mixture of HCO in coconut oil showed TM4 at 68.2° C. Mixtures of HCO in isopropyl myristate and MCT oil showed TM4 peak at 65.1° C. and 65.3° C., respectively.

Mixtures of other oils (octyldodecanol, diisopropyl adipate, corn oil, and safflower oil) with hydrogenated castor oil (HCO) with APIs (MCH and ADP) are prepared (Table 37B) in a continuous-heating cooling process and analyzed by DSC.

TABLE 37A

| Component | | | | | | |
|---|---|---|---|---|---|---|
| Soybean oil | | 95.5 | | | | |
| Coconut oil | | | 95.5 | | | |
| Isopropyl myristate | | | | 95.5 | | |
| Light mineral oil | | | | | 95.5 | |
| MCT oil | | | | | | 95.5 |
| Hydrogenated castor oil | | 1.2 | 1.2 | 1.2 | 1.2 | 1.2 |
| MCH | | 3 | 3 | 3 | 3 | 3 |
| ADP | | 0.3 | 0.3 | 0.3 | 0.3 | 0.3 |
| Total | | 100 | 100 | 100 | 100 | 100 |
| Test | Temperature | | | | | |
| DSC TM3 | 25° C. | 44.4 | 41.5 | 40.6 | | 44.5 |
| DSC | 25° C. | | | | 52.3 | |
| TM4.2 | | | | | | |
| DSC | 25° C. | | 55.3 | 56.0 | | 57.4 |
| TM4.1 | | | | | | |
| DSC TM4 | 25° C. | 72.3 | 68.2 | 65.1 | 80.2 | 65.3 |

TABLE 37B

| Component | | | | |
|---|---|---|---|---|
| Octyldodecanol | 95.5 | | | |
| Di-isopropyl adipate | | 95.5 | | |
| Corn oil | | | 95.5 | |
| Safflower oil | | | | 95.5 |
| Hydrogenated castor oil | 1.2 | 1.2 | 1.2 | 1.2 |
| MCH | 3 | 3 | 3 | 3 |
| ADP | 0.3 | 0.3 | 0.3 | 0.3 |
| Total | 100 | 100 | 100 | 100 |

### Example 25

Effect of Adapalene Alone, MCH Alone, their Combination or a Formulation Vehicle on Expression of Endogenous Antimicrobial Peptides in a 3D Skin Model/Ex-Vivo Human Skin

Formulations containing 1.2% hydrogenated castor oil (HCO), with 3% minocycline HCl, and 0.3% adapalene (MCH+ADP), with MCH alone, with ADP alone, or without APIs are prepared by a holding process or a continuous heating-cooling process and tested for induction of anti-microbial peptides.

TABLE 38

| Study | | | | | |
|---|---|---|---|---|---|
| Formulation # | | | | | |
| HCO amount | 1.2% | 1.2% | 1.2% | 1.2% | 1.2% |
| API | MCH + ADP | MCH + ADP | MCH | ADP | Placebo |
| Process | Holding | Continuous | Holding | Holding | Holding |
| Component | | | | | |
| Soybean oil | 50 | 50 | 50 | 50 | 50 |
| coconut oil | 23.6 | 23.6 | 23.6 | 23.6 | 23.6 |
| Light mineral oil* | 3.3 | 3.3 | 3.6 | 6.3 | 6.6 |
| Stearic acid | 3 | 3 | 3 | 3 | 3 |
| Docosanol | 1.1 | 1.1 | 1.1 | 1.1 | 1.1 |
| Hydrogenated castor | 1.2 | 1.2 | 1.2 | 1.2 | 1.2 |
| oil | | | | | |
| White wax (beeswax) | 2 | 2 | 2 | 2 | 2 |
| Stearyl alcohol | 1.5 | 1.5 | 1.5 | 1.5 | 1.5 |
| Cetostearyl alcohol | 3.5 | 3.5 | 3.5 | 3.5 | 3.5 |
| Myristyl alcohol | 2.5 | 2.5 | 2.5 | 2.5 | 2.5 |
| Cyclomethicone | 5 | 5 | 5 | 5 | 5 |
| Minocycline HCI** | 3 | 3 | 3 | 0 | 0 |
| Adapalene | 0.3 | 0.3 | 0 | 0.3 | 0 |
| TOTAL | 100 | 100 | 100 | 100 | 100 |

| Test | Temperature |
|---|---|
| LL-37 | 5° C. |
| | 25° C. |
| | 40° C. |
| β defensin | 5° C. |
| | 25° C. |
| | 40° C. |
| RNase7 | 5° C. |
| | 25° C. |
| | 40° C. |

*The amount of light mineral oil in the formulation is adjusted based on the amount of minocycline hydrochloride and/or adapalene.
**The amount of minocycline hydrochloride in the formulation is adjusted by the potency of the minocycline hydrochloride.

### Example 26

### Antibiotic Resistance

Formulations containing 1.2% hydrogenated castor oil (HCO), with 3% minocycline HCl, and 0.3% adapalene (MCH+ADP), with MCH alone, or with ADP alone are prepared in a holding process or a continuous heating-cooling process and tested for antibiotic resistance

TABLE 39

| Study | | | | |
|---|---|---|---|---|
| Formulation # | | | | |
| HCO amount | 1.2% | 1.2% | 1.2% | 1.2% |
| API | MCH+ADP | MCH+ADP | MCH | ADP |
| Process | Continuous | Holding | Holding | Holding |
| Component | | | | |
| Soybean oil | 50 | 50 | 50 | 50 |
| coconut oil | 23.6 | 23.6 | 23.6 | 23.6 |
| Light mineral oil* | 3.3 | 3.3 | 3.6 | 6.3 |
| Stearic acid | 3 | 3 | 3 | 3 |
| Docosanol | 1.1 | 1.1 | 1.1 | 1.1 |
| Hydrogenated castor oil | 1.2 | 1.2 | 1.2 | 1.2 |
| White wax (beeswax) | 2 | 2 | 2 | 2 |
| Stearyl alcohol | 1.5 | 1.5 | 1.5 | 1.5 |
| Cetostearyl alcohol | 3.5 | 3.5 | 3.5 | 3.5 |
| Myristyl alcohol | 2.5 | 2.5 | 2.5 | 2.5 |
| Cyclomethicone | 5 | 5 | 5 | 5 |
| Minocycline HCl** | 3 | 3 | 3 | 0 |
| Adapalene | 0.3 | 0.3 | 0 | 0.3 |
| TOTAL | 100 | 100 | 100 | 100 |

| Test | Temperature | |
|---|---|---|
| Minimal | 5° | C. |
| inhibitory | 25° | C. |
| concentration | 40° | C. |

*The amount of light mineral oil in the formulation is adjusted based on the amount of minocycline hydrochloride and/or adapalene.
**The amount of minocycline hydrochloride in the formulation is adjusted by the potency of the minocycline hydrochloride.

### Example 27

### In-Vitro Release Testing

An In-Vitro Release Testing (IVRT) study was conducted to characterize the release rate of minocycline and adapalene from various formulations (Table 40A).

TABLE 40A
Composition of Investigated Formulations

| | FCDI05 | FCD105 | FCD105 | FCD105 | FCD105 | FCD105 | FCD105 | FCD105 | FCD105 | FCD105 | FCD 105 | FCD105 |
|---|---|---|---|---|---|---|---|---|---|---|---|---|
| | (3,0.3)- | (3,0.3)- | (3,0.3)- | (0,0.3)- | (3,0.3)- | (3,0)- | (3,0.3)- | (0,0.3)- | (3,0.3)- | (3,0.3)- | (3,0.3)- | (3,0.3)- |
| Batch No. | 191229S | 191111S | 191124R | 190317H | 190319S | 190319H | 190505S | 190403S | 190430R | 190710S | 191229R | 191216R |
| Process | Holding | Holding | Holding | Continuous | Continuous | Continuous | Holding | Holding | Holding | Holding | Holding | Holding |
| Coconut oil | x | 23.60 | 23.60 | 23.6 | 23.60 | 23.6 | 23.60 | 23.6 | 23.60 | x | 3.6 | 50 |
| Isopropyl | x | x | x | x | x | x | x | x | x | 23.60 | x | x |
| Myristate | | | | | | | | | | | | |
| MCT Oil | 23.60 | x | x | x | x | x | x | x | x | x | x | x |
| Light mineral oil | 2.72 | 3.82 | 1.92 | 6.3 | 2.72 | 3.02 | 2.72 | 6.3 | 1.92 | 2.72 | 2.72 | 2.82 |
| Soybean oil | 50.00 | 50.00 | 50.00 | 50.00 | 50.00 | 50.00 | 50.00 | 50.00 | 50.00 | 50.00 | 70.00 | 23.50 |
| Stearic acid | 3.00 | 3.00 | 3.00 | 3 | 3.00 | 3.00 | 3.00 | 3.0 | 3.00 | 3.00 | 3.00 | 3.00 |
| Docosanol | 1.10 | 1.10 | 1.10 | 1.1 | 1.10 | 1.10 | 1.10 | 1.1 | 1.10 | 1.10 | 1.10 | 1.10 |
| Hydrogenated | 1.20 | 0.10 | x | 1.2 | 1.20 | 1.20 | 1.20 | 1.2 | 2.00 | 1.2 | 1.2 | 1.2 |
| castor oil | | | | | | | | | | | | |
| Emulsifying wax | x | x | 2.00 | x | x | x | x | x | x | x | x | x |
| White wax | 2.00 | 2.00 | 2.00 | 2.00 | 2.00 | 2.00 | 2.00 | 2.00 | 2.00 | 2.00 | 2.00 | 2.00 |
| Stearyl alcohol | 1.50 | 1.50 | 1.50 | 1.5 | 1.50 | 1.50 | 1.50 | 1.5 | 1.50 | 1.50 | 1.50 | 1.50 |
| Cetostearyl | 3.50 | 3.50 | 3.50 | 3.5 | 3.50 | 3.50 | 3.50 | 3.5 | 3.50 | 3.50 | 3.50 | 3.50 |
| alcohol | | | | | | | | | | | | |
| Myristyl alcohol | 2.50 | 2.50 | 2.50 | 2.5 | 2.50 | 2.50 | 2.50 | 2.5 | 2.50 | 2.50 | 2.50 | 2.50 |
| Adapalene | 0.30 | 0.30 | 0.30 | 0.3 | 0.30 | 0.30 | 0.30 | 0.3 | 0.30 | 0.30 | 0.30 | 0.30 |
| Cyclomethicone | 5.00 | 5.00 | 5.00 | 5.00 | 5.00 | 5.00 | 5.00 | 5.00 | 5.00 | 5.00 | 5.00 | 5.00 |
| Minocycline HCI | 3.58 | 3.58 | 3.58 | x | 3.58 | 3.58 | 3.58 | x | 3.58 | 3.58 | 3.58 | 3.58 |

### Methods

IVRT was performed using a Franz-cell apparatus. Each tested formulation was placed in three replicates on suitable membranes, and a suitable receptor fluid was placed in the receptor chamber. The concentration of the active agent(s) in the receptor fluid was measured over time over the course of 6 hours. The amount of drug released is typically proportional to the square root of time; therefore, a plot of the cumulative release vs. time 0.5 yields a straight line, the slope of which is used to calculate the release rate (amount released/cm²/min^{0.5}).

### Results

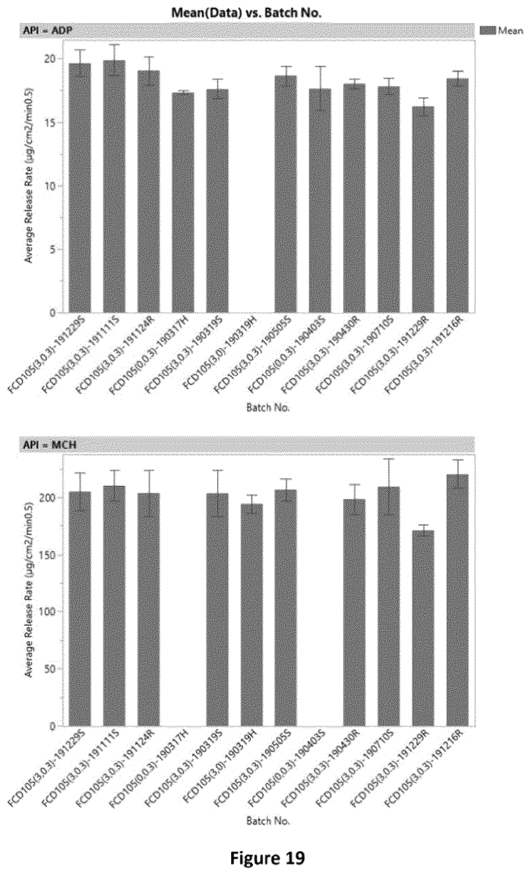

Tabulated results and histograms of the average release rate for minocycline and adapalene are presented respectively in Table 40B and FIG. 19.

TABLE 40B
Average release rate results for minocycline and adapalene

| | Average Release rate | Average Release rate |
|---|---|---|
| | Minocycline | Adapalene |
| Batch No. | (μg/cm²/min^{0.5}) | (μg/cm²/min^{0.5}) |
| FCD105(3,0.3)- | 205.16 | 19.65 |
| 191229S | | |
| FCD105(3,0.3)- | 210.52 | 19.89 |
| 191111S | | |
| FCD105(3,0.3)- | 203.71 | 19.04 |
| 191124R | | |
| FCD105(0,0.3)- | NA | 17.35 |
| 190317H | | |
| FCD105(3,0.3)- | 203.68 | 17.63 |
| 190319S | | |
| FCD105(3,0)-190319H | 194.41 | NA |
| FCD105(3,0.3)- | 206.80 | 18.67 |
| 190505S | | |
| FCD105(0,0.3)- | NA | 17.65 |
| 190403S | | |
| FCD105(3,0.3)- | 198.63 | 18.05 |
| 190430R | | |
| FCD105(3,0.3)- | 209.46 | 17.85 |
| 190710S | | |
| FCD105(3,0.3)- | 171.09 | 16.24 |
| 191229R | | |
| FCD105(3,0.3)- | 220.22 | 18.46 |
| 191216R | | |

### Discussion

The comparison of the release rate results shows that a batch made with a continuous process (FCD105(3,0.3)-190319S) had a similar release rate as a batch made with a holding process of 4 hours at 54C (FCD105(3,0.3)-190505S). No significant differences in the release rates could be noticed between batches comprising both APIs (FCD105(3,0.3)-190319S) or only one of the APIs (FCD105(0,0.3)-190317H; FCD105(3,0)-190319H). No significant differences in the release rates could be noticed when coconut oil was replaced by MCT oil (FCD105(3,0.3)-191229S) or by isopropyl myristate (FCD105(3,0.3)-19071OS), or when a different beeswax to HCO ratio was used (FCD105(3,0.3)-191111 S; FCD105(3,0.3)-190430R), or when 2% emulsifying wax was used instead of HCO (FCD105(3,0.3)-191124R).

When the concentration of coconut oil was reduced from 23.6% to 3.6% (FCD105(3,0.3)-191229R), the release rate of minocycline and adapalene was reduced. When the concentration of coconut oil was increased from 23.6% to 50% (FCD105(3,0.3)-191216R), the release rate of minocycline was increased, but not that of adapalene.

Accordingly, the concentration of coconut oil in the drug product appears to have an impact on the release rate of minocycline and to some extent of adapalene. Increased amounts of coconut oil enable a faster release of minocycline from the composition, and thus potentially an increase in the skin penetration of minocycline and the efficacy of the drug product in treating skin diseases. Without being bound by theory varying the amount of oils like coconut oil in the formulation can be used to adjust the amount of delivery of drug into the skin and release to the dermis and epidermis and thereby the efficacy of the drug.

### Example 28: Skin Penetration

Formulations comprising 1.2% HCO, with 3% minocycline HCl, and 0.3% adapalene (MCH+ADP), are prepared by a holding process or a continuous heating-cooling process. Skin penetration is measured in a flow through system and or a Franz Cell system as described above.

### Example 29A: Compatibility Studies

Formulations containing different oils, either 50% safflower oil or 50% corn oil (202505R and 202505D, in Table 35) with MCH+ADP were prepared and tested for compatibility. Formulations were stored for 10 weeks at 40° C. and observed for physical and chemical compatibility following dispensing of the foam. In both formulations containing 50% corn oil and 50% safflower oil, a foam of excellent quality having a yellow color was produced evidencing chemical stability of minocycline. This confirms the physical compatibility of the two oils within both foam formulations.

### Example 29B: Compatibility Studies

Mixtures of solid Minocycline HCl and Adapalene are added to neat soybean oil, neat corn oil, or neat safflower oil. The exact amount of Minocycline HCl and Adapalene are determined for each sample by weighing the active ingredients with an analytical balance. The mixtures are placed into glass screw cap vials, tightly closed, and exposed to 50° C. temperatures for 3 weeks, protected from light. Following 3 weeks of exposure, the mixtures are equilibrated with ambient conditions and Minocycline, Adapalene, and their degradation products are determined by HPLC, analyzing the complete samples. The extent of degradation of Minocycline and Adapalene is determined for each sample by comparison of the amounts of Minocycline HCl and Adapalene recovered in each sample with the weights of Minocycline HCl and Adapalene used in preparing the corresponding samples. The content of degradation products is determined by area percent ratio for each degradation product to the main peak of the corresponding active ingredient. The samples are evaluated for color alterations, as well.

TABLE 41

| Ingredient | Ingredient content, % w/w | | |
|---|---|---|---|
| Adapalene | 0.3 | 0.3 | 0.3 |
| Minocycline HCl* | 3.58 | 3.58 | 3.58 |
| Soybean oil | qs to 100 | | |
| Corn oil | | qs to 100 | |
| Safflower oil | | | qs to 100 |
| Bulk Total | 100 | 100 | 100 |

*3.58% of Minocycline HCl corresponds to 3.0% of Minocycline

### Example 30: Mixtures Prepared by Stepwise Addition of Formulation Components in a Holding Process

Different mixtures are created by stepwise addition of individual components in the order presented in Table 42A and in the amounts shown in Table 42B. Upon addition of each component, the mixture is first melted at 90° C. for 20 minutes, then cooled to a temperature of about 540 and is kept at this temperature for about 4 hours. The mixture is then further cooled (at 10° C./min) to 5° C.. After 2 minutes at 5° C., the mixture is heated from 5° C. up to 90° C. (at 5° C./min). In this way, the melting profile of each of the component scan be characterized. In one experiment, the above process and mixing for each of the steps is conducted outside the DSC except for the last part of each step where, after cooling to 5° C., a sample is paced in the DSC and is heated from 5° C. up to 90° C. (at 5° C./min).

TABLE 42A

| STEP | SAMPLE | TM0 | TM1 | TM2 | TM2.1 | TM3 | TM4 |
|---|---|---|---|---|---|---|---|
| 1 | Oils + | | | | | | |
| | cyclomethicone | | | | | | |
| | (CM) | | | | | | |
| 2 | +Hydrogenated | | | | | | |
| | castor oil (HCO) | | | | | | |
| 3 | +Docosanol | | | | | | |
| 4 | +Beeswax | | | | | | |
| 5 | +Stearyl alcohol | | | | | | |
| 6 | +Stearic acid | | | | | | |
| 7 | +Cetostearyl | | | | | | |
| | alcohol | | | | | | |
| 8 | +Myristyl | | | | | | |
| | alcohol | | | | | | |
| 9 | Full placebo | | | | | | |
| | formulation | | | | | | |
| | (PFPB) | | | | | | |

TABLE 42B

| Component | % w/w |
|---|---|
| Soybean oil | 50 |
| coconut oil | 23.6 |
| Light mineral oil | 5.8 |
| Stearic acid | 3 |
| Docosanol | 1.1 |
| Hydrogenated castor oil | 2 |
| White wax (beeswax) | 2 |
| Stearyl alcohol | 1.5 |
| Cetostearyl alcohol | 3.5 |
| Myristyl alcohol | 2.5 |
| Cyclomethicone | 5 |

### Example 31. Clinical Trial Results

In this example, a phase 2 clinical evaluation is reported for treatment batches prepared using continual high shear during the manufacturing process, including during the holding step. Preliminary evaluation of samples of the compositions prepared with shear suggests Tmh crystals were not visible or only visible in low numbers. Preliminary evaluation of these compositions by DSC and microscopy showed that these batches had a TM4 value in the range of 63-64° C.

Positive results were observed from a Phase 2 clinical trial evaluating the preliminary safety and efficacy of FCD105 (3% minocycline and 0.3% adapalene foam), the first ever topical minocycline-based combination product, for the treatment of moderate-to-severe acne vulgaris. Study FX2016-40 enrolled 447 patients in the United States that were randomized to either FCD105 foam, 3% minocycline foam, 0.3% adapalene foam or vehicle foam. Safety population and PP population included 446 and 445 subjects respectively. 417 (93.3%) subjects completed 12 weeks of treatment. (see Table 43A).

Subjects who were enrolled in 35 sites and were randomized (5:4:4:3) to receive to either combination foam FCD105, 3% minocycline foam, 0.3% adapalene foam or vehicle respectively once daily (1 hour before bedtime) over 12 weeks. Subjects self-apply treatment to the full face and other acne affected areas. The study medication, dosage, inclusion/exclusion criteria, and design generally followed those outlined in Example 20 above, with the inclusion criteria of healthy males or non-pregnant female aged 12 or older, having at least 20-50 inflammatory lesions, 25-100 non-inflammatory lesions, and having the Investigator's Global Assessment (IGA) scores moderate or severe with no more than 2 active nodules on the face.

The mean age of the study participants in the ITT, Safety and PP populations was 21.3 and 61% of the participants were female. The mean baseline inflammatory and non-inflammatory lesion count for all groups was 30.6 and 48.1 respectively and the IGA scores were all moderate (score 3) or severe (score 4), with 90.8% of the subjects having a moderate rating (see Table 43B-43D). 35% of the subjects ITT, Safety and PP populations received concomitant medications. The number of subjects reporting concomitant medications was similar among the treatment arms, including 44 subjects (31.0%) in the FCD105 arm, 25 subjects (30.1%) in the vehicle arm, 41 subjects (37.3%) in the minocycline 3% arm, and 47 subjects (42.0%) in the adapalene 0.3% arm. The most commonly used class of concomitant medications was sex hormones and modulators of the genital system (used in 45 subjects [10.1%] overall; 9.9% of subjects in the FCD105 arm, 7.2% of subjects in the vehicle arm, 6.4% of subjects in the minocycline 3% arm, and 16.1% of subjects in the adapalene 0.3% arm). Other commonly used concomitant medications, including other gynecologicals, psychoanaleptics, vitamins, analgesics, anti-inflammatory and antirheumatic products, and antihistamines for systemic use, were used in <5% of subjects overall, and use was generally similar among the treatment arms. Emollients and protectives were also used by 16 subjects (5 subjects [3.5%] in FCD105 arm, 0 subjects in the vehicle arm, 5 subjects [4.5%] in the minocycline 3% arm, and 6 subjects [5.4%] in the adapalene 0.3% arm).

The co-primary efficacy endpoints were the absolute change from Baseline in inflammatory and non-inflammatory lesion counts at Week 12 (first primary endpoint), and proportion of subjects (%) with IGA score of 0/1 (“Clear” or “Minimal”) at Week 12 (second primary endpoint). The secondary efficacy endpoints were (i) percent change from Baseline in the inflammatory and non-inflammatory lesion counts at Weeks 12, 8 and 4 (ii) IGA Treatment Success of IGA score of 0 or 1 and at least a 2-grade improvement (decrease) at Weeks 8 and 4. FCD105 vs. 0.3% adapalene foam, and (iii) FCD105 vs. 3% minocycline foam or 0.3% adapalene foam for all co-primary endpoints at Week 12. Safety and tolerability in the treatment of moderate to severe acne vulgaris were also evaluated. Safety and efficacy evaluations were performed at baseline and week 4, 8, and 12, with an optional additional safety follow-up visit at week 16 which included Treatment Emergent Adverse Events (TEAE), Local Skin Tolerability Assessments (TSLA) (burning/stinging, itching, dryness, scaling, erythema and hyperpigmentation), vital signs, and physical examination. Subject satisfaction was evaluated at the end of the study based on a subject satisfaction questionnaire.

TABLE 43A
Subject Enrollment and Disposition-All Subjects

| | FCD105 | Vehicle | Minocycline 3% | Adapalene 0.3% | Total |
|---|---|---|---|---|---|
| Screened | | | | | 500 |
| Population | | | | | |
| Screen Failure | | | | | 53 |
| Population ^{[1]} | | | | | |
| Randomized | 142 | 83 | 110 | 112 | 447 |
| Population | | | | | |
| ITT Population ^{[2]} | 142 | 83 | 110 | 112 | 447 |
| Safety Population ^{[3]} | 142 | 82 | 110 | 112 | 446 |
| PP Population ^{[4]} | 142 | 82 | 110 | 111 | 445 |
| Completed Study | 130 (91.5%) | 80 (96.4%) | 106 (96.4%) | 101 (90.2%) | 417 (93.3%) |
| Withdrawal from | 12 (8.5%) | 3 (3.6%) | 4 (3.6%) | 11 (9.8%) | 30 (6.7%) |
| Study | | | | | |

Primary Reason for Withdrawal

| | | | | | |
|---|---|---|---|---|---|
| Adverse Event | 1 (0.7%) | 0 | 0 | 3 (2.7%) | 4 (0.9%) |
| Lost to Follow-up | 6 (4.2%) | 1 (1.2%) | 2 (1.8%) | 2 (1.8%) | 11 (2.5%) |
| Physician Decision | 0 | 0 | 0 | 0 | 0 |
| Protocol Deviation | 0 | 0 | 0 | 0 | 0 |
| Withdrawal by | 4 (2.8%) | 1 (1.2%) | 1 (0.9%) | 6 (5.4%) | 12 (2.7%) |
| Subject | | | | | |
| Other, Specify | 1 (0.7%) | 1 (1.2%) | 1 (0.9%) | 0 | 3 (0.7%) |

^{[1]} All subjects who signed the informed consent but were not randomized to any treatment group.
^{[2]} All randomized subjects who have completed baseline assessments.
^{[3]} All randomized subjects who took at least one dose of study drug. Subjects who have no post-Baseline assessments will be included in the Safety population unless all dispensed study drug is returned unused.
^{[4]} All subjects in the ITT population without any protocol deviations that may have an impact on the efficacy assessments.
Note:
The denominator for the percentages is the number of subjects (n) in the ITT population.

TABLE 43B
Demographics and Baseline Characteristics-ITT Populations

| | | | Minocycline | Adapalene | |
|---|---|---|---|---|---|
| | FCD105 | Vehicle | 3% | 0.3% | Total |
| | (N = 142) | (N = 83) | (N = 110) | (N = 112) | (N = 447) |
| Age (years) | | | | | |
| n | 142 | 83 | 110 | 112 | 447 |
| Mean (SD) | 21.0 (7.05) | 21.4 (7.25) | 20.8 (8.28) | 22.0 (7.98) | 21.3 (7.63) |
| Median | 19.0 | 19.0 | 17.5 | 20.0 | 19.0 |
| Min, Max | 12, 43 | 12, 41 | 12, 60 | 12, 64 | 12, 64 |
| Age Groups | | | | | |
| <18 years | 64 (45.1%) | 32 (38.6%) | 55 (50.0%) | 41 (36.6%) | 192 (43.0%) |
| 18-40 years | 76 (53.5%) | 50 (60.2%) | 52 (47.3%) | 70 (62.5%) | 248 (55.5%) |
| 41-64 years | 2 (1.4%) | 1 (1.2%) | 3 (2.7%) | 1 (0.9%) | 7 (1.6%) |
| 65-75 years | 0 | 0 | 0 | 0 | 0 |
| >75 years | 0 | 0 | 0 | 0 | 0 |
| Sex | | | | | |
| Male | 62 (43.7%) | 33 (39.8%) | 43 (39.1%) | 36 (32.1%) | 174 (38.9%) |
| Female | 80 (56.3%) | 50 (60.2%) | 67 (60.9%) | 76 (67.9%) | 273 (61.1%) |
| Ethnicity | | | | | |
| Hispanic or Latino | 63 (44.4%) | 33 (39.8%) | 47 (42.7%) | 43 (38.4%) | 186 (41.6%) |
| Not Hispanic or Latino | 79 (55.6%) | 50 (60.2%) | 63 (57.3%) | 69 (61.6%) | 261 (58.4%) |
| Race | | | | | |
| American Indian or Alaska | 1 (0.7%) | 0 | 0 | 0 | 1 (0.2%) |
| Native | | | | | |
| Asian | 7 (4.9%) | 6 (7.2%) | 5 (4.5%) | 2 (1.8%) | 20 (4.5%) |
| Black or African American | 29 (20.4%) | 15 (18.1%) | 23 (20.9%) | 27 (24.1%) | 94 (21.0%) |
| Native Hawaiian or other | 0 | 1 (1.2%) | 0 | 0 | 1 (0.2%) |
| Pacific Islander | | | | | |
| White | 103 (72.5%) | 56 (67.5%) | 81 (73.6%) | 76 (67.9%) | 316 (70.7%) |
| Multiple | 0 | 4 (4.8%) | 0 | 4 (3.6%) | 8 (1.8%) |
| Not Reported | 2 (1.4%) | 1 (1.2%) | 1 (0.9%) | 3 (2.7%) | 7 (1.6%) |
| Weight (kg) | | | | | |
| n | 142 | 82 | 110 | 112 | 446 |
| Mean (SD) | 72.68 | 75.18 | 69.93 | 77.06 | 73.56 |
| | (21.322) | (21.516) | (16.194) | (24.619) | (21.232) |
| Median | 66.10 | 70.25 | 67.15 | 71.10 | 68.40 |
| Min, Max | 37.5, 138.8 | 40.8, 133.4 | 40.4, 110.7 | 43.5, 163.3 | 37.5, 163.3 |
| Height (cm) | | | | | |
| n | 142 | 82 | 110 | 112 | 446 |
| Mean (SD) | 166.87 | 167.34 | 166.05 | 166.45 | 166.65 |
| | (10.802) | (10.330) | (9.451) | (9.146) | (9.967) |
| Median | 165.10 | 165.10 | 165.00 | 167.60 | 165.10 |
| Min, Max | 129.5, 190.5 | 125.2, 193.0 | 143.0, 188.0 | 124.5, 188.0 | 124.5, 193.0 |
| BMI (kg/m2) | | | | | |
| n | 142 | 82 | 110 | 112 | 446 |
| Mean (SD) | 26.10 (7.429) | 26.81 (7.157) | 25.37 (5.654) | 27.74 (8.256) | 26.46 (7.240) |
| Median | 24.10 | 24.90 | 23.55 | 25.70 | 24.30 |
| Min, Max | 15.6, 54.2 | 17.2, 50.2 | 16.6, 44.6 | 17.6, 59.0 | 15.6, 59.0 |
| Baseline inflammatory | | | | | |
| lesion count | | | | | |
| n | 142 | 83 | 110 | 112 | 447 |
| Mean (SD) | 30.2 (7.61) | 30.0 (8.14) | 31.0 (8.71) | 31.1 (8.58) | 30.6 (8.22) |
| Median | 27.5 | 28.0 | 28.0 | 29.0 | 28.0 |
| Min, Max | 20, 50 | 20, 50 | 20, 50 | 20, 50 | 20, 50 |
| Baseline non-inflammatory | | | | | |
| lesion count | | | | | |
| n | 142 | 83 | 110 | 112 | 447 |
| Mean (SD) | 47.2 (16.74) | 49.8 (16.51) | 48.4 (19.08) | 47.8 (16.85) | 48.1 (17.30) |
| Median | 44.5 | 46.0 | 42.5 | 44.5 | 45.0 |
| Min, Max | 25, 94 | 26, 86 | 25, 99 | 25, 98 | 25, 99 |
| Baseline IGA score | | | | | |
| Moderate | 134 (94.4%) | 76 (91.6%) | 96 (87.3%) | 100 (89.3%) | 406 (90.8%) |
| Severe | 8 (5.6%) | 7 (8.4%) | 14 (12.7%) | 12 (10.7%) | 41 (9.2%) |

TABLE 43C
Demographics and Baseline Characteristics-Safety Population

| | | | Minocycline | Adapalene | |
|---|---|---|---|---|---|
| | FCD105 | Vehicle | 3% | 0.3% | Total |
| | (N = 142) | (N = 82) | (N = 110) | (N = 112) | (N = 446) |
| Age (years) | | | | | |
| n | 142 | 82 | 110 | 112 | 446 |
| Mean (SD) | 21.0 (7.05) | 21.4 (7.30) | 20.8 (8.28) | 22.0 (7.98) | 21.3 (7.64) |
| Median | 19.0 | 19.0 | 17.5 | 20.0 | 19.0 |
| Min, Max | 12, 43 | 12, 41 | 12, 60 | 12, 64 | 12, 64 |
| Age Groups | | | | | |
| <18 years | 64 (45.1%) | 32 (39.0%) | 55 (50.0%) | 41 (36.6%) | 192 (43.0%) |
| 18-40 years | 76 (53.5%) | 49 (59.8%) | 52 (47.3%) | 70 (62.5%) | 247 (55.4%) |
| 41-64 years | 2 (1.4%) | 1 (1.2%) | 3 (2.7%) | 1 (0.9%) | 7 (1.6%) |
| 65-75 years | 0 | 0 | 0 | 0 | 0 |
| >75 years | 0 | 0 | 0 | 0 | 0 |
| Sex | | | | | |
| Male | 62 (43.7%) | 33 (40.2%) | 43 (39.1%) | 36 (32.1%) | 174 (39.0%) |
| Female | 80 (56.3%) | 49 (59.8%) | 67 (60.9%) | 76 (67.9%) | 272 (61.0%) |
| Ethnicity | | | | | |
| Hispanic or Latino | 63 (44.4%) | 32 (39.0%) | 47 (42.7%) | 43 (38.4%) | 185 (41.5%) |
| Not Hispanic or Latino | 79 (55.6%) | 50 (61.0%) | 63 (57.3%) | 69 (61.6%) | 261 (58.5%) |
| Race | | | | | |
| American Indian or Alaska | 1 (0.7%) | 0 | 0 | 0 | 1 (0.2%) |
| Native | | | | | |
| Asian | 7 (4.9%) | 6 (7.3%) | 5 (4.5%) | 2 (1.8%) | 20 (4.5%) |
| Black or African American | 29 (20.4%) | 15 (18.3%) | 23 (20.9%) | 27 (24.1%) | 94 (21.1%) |
| Native Hawaiian or other | 0 | 1 (1.2%) | 0 | 0 | 1 (0.2%) |
| Pacific Islander | | | | | |
| White | 103 (72.5%) | 55 (67.1%) | 81 (73.6%) | 76 (67.9%) | 315 (70.6%) |
| Multiple | 0 | 4 (4.9%) | 0 | 4 (3.6%) | 8 (1.8%) |
| Not Reported | 2 (1.4%) | 1 (1.2%) | 1 (0.9%) | 3 (2.7%) | 7 (1.6%) |
| Weight (kg) | | | | | |
| n | 142 | 82 | 110 | 112 | 446 |
| Mean (SD) | 72.68 | 75.18 | 69.93 | 77.06 | 73.56 |
| | (21.322) | (21.516) | (16.194) | (24.619) | (21.232) |
| Median | 66.10 | 70.25 | 67.15 | 71.10 | 68.40 |
| Min, Max | 37.5, 138.8 | 40.8, 133.4 | 40.4, 110.7 | 43.5, 163.3 | 37.5, 163.3 |
| Height (cm) | | | | | |
| n | 142 | 82 | 110 | 112 | 446 |
| Mean (SD) | 166.87 | 167.34 | 166.05 | 166.45 | 166.65 |
| | (10.802) | (10.330) | (9.451) | (9.146) | (9.967) |
| Median | 165.10 | 165.10 | 165.00 | 167.60 | 165.10 |
| Min, Max | 129.5, 190.5 | 125.2, 193.0 | 143.0, 188.0 | 124.5, 188.0 | 124.5, 193.0 |
| BMI (kg/m2) | | | | | |
| n | 142 | 82 | 110 | 112 | 446 |
| Mean (SD) | 26.10 (7.429) | 26.81 (7.157) | 25.37 (5.654) | 27.74 (8.256) | 26.46 (7.240) |
| Median | 24.10 | 24.90 | 23.55 | 25.70 | 24.30 |
| Min, Max | 15.6, 54.2 | 17.2, 50.2 | 16.6, 44.6 | 17.6, 59.0 | 15.6, 59.0 |
| Baseline inflammatory | | | | | |
| lesion count | | | | | |
| n | 142 | 82 | 110 | 112 | 446 |
| Mean (SD) | 30.2 (7.61) | 30.1 (8.18) | 31.0 (8.71) | 31.1 (8.58) | 30.6 (8.23) |
| Median | 27.5 | 28.0 | 28.0 | 29.0 | 28.0 |
| Min, Max | 20, 50 | 20, 50 | 20, 50 | 20, 50 | 20, 50 |
| Baseline non-inflammatory | | | | | |
| lesion count | | | | | |
| n | 142 | 82 | 110 | 112 | 446 |
| Mean (SD) | 47.2 (16.74) | 49.9 (16.59) | 48.4 (19.08) | 47.8 (16.85) | 48.1 (17.31) |
| Median | 44.5 | 46.0 | 42.5 | 44.5 | 45.0 |
| Min, Max | 25, 94 | 26, 86 | 25, 99 | 25, 98 | 25, 99 |
| Baseline IGA score | | | | | |
| Moderate | 134 (94.4%) | 75 (91.5%) | 96 (87.3%) | 100 (89.3%) | 405 (90.8%) |
| Severe | 8 (5.6%) | 7 (8.5%) | 14 (12.7%) | 12 (10.7%) | 41 (9.2%) |

TABLE 43D
Demographics and Baseline Characteristics-Per-Protocol
Population

| | | | Minocycline | Adapalene | |
|---|---|---|---|---|---|
| | FCD105 | Vehicle | 3% | 0.3% | Total |
| | (N = 142) | (N = 82) | (N = 110) | (N = 111) | (N = 445) |
| Age (years) | | | | | |
| n | 142 | 82 | 110 | 111 | 445 |
| Mean (SD) | 21.0 (7.05) | 21.4 (7.30) | 20.8 (8.28) | 22.0 (8.01) | 21.3 (7.64) |
| Median | 19.0 | 19.0 | 17.5 | 20.0 | 19.0 |
| Min, Max | 12, 43 | 12, 41 | 12, 60 | 12, 64 | 12, 64 |
| Age Groups | | | | | |
| <18 years | 64 (45.1%) | 32 (39.0%) | 55 (50.0%) | 41 (36.9%) | 192 (43.1%) |
| 18-40 years | 76 (53.5%) | 49 (59.8%) | 52 (47.3%) | 69 (62.2%) | 246 (55.3%) |
| 41-64 years | 2 (1.4%) | 1 (1.2%) | 3 (2.7%) | 1 (0.9%) | 7 (1.6%) |
| 65-75 years | 0 | 0 | 0 | 0 | 0 |
| >75 years | 0 | 0 | 0 | 0 | 0 |
| Sex | | | | | |
| Male | 62 (43.7%) | 33 (40.2%) | 43 (39.1%) | 36 (32.4%) | 174 (39.1%) |
| Female | 80 (56.3%) | 49 (59.8%) | 67 (60.9%) | 75 (67.6%) | 271 (60.9%) |
| Ethnicity | | | | | |
| Hispanic or Latino | 63 (44.4%) | 32 (39.0%) | 47 (42.7%) | 42 (37.8%) | 184 (41.3%) |
| Not Hispanic or Latino | 79 (55.6%) | 50 (61.0%) | 63 (57.3%) | 69 (62.2%) | 261 (58.7%) |
| Race | | | | | |
| American Indian or Alaska | 1 (0.7%) | 0 | 0 | 0 | 1 (0.2%) |
| Native | | | | | |
| Asian | 7 (4.9%) | 6 (7.3%) | 5 (4.5%) | 2 (1.8%) | 20 (4.5%) |
| Black or African American | 29 (20.4%) | 15 (18.3%) | 23 (20.9%) | 27 (24.3%) | 94 (21.1%) |
| Native Hawaiian or other | 0 | 1 (1.2%) | 0 | 0 | 1 (0.2%) |
| Pacific Islander | | | | | |
| White | 103 (72.5%) | 55 (67.1%) | 81 (73.6%) | 75 (67.6%) | 314 (70.6%) |
| Multiple | 0 | 4 (4.9%) | 0 | 4 (3.6%) | 8 (1.8%) |
| Not Reported | 2 (1.4%) | 1 (1.2%) | 1 (0.9%) | 3 (2.7%) | 7 (1.6%) |
| Weight (kg) | | | | | |
| n | 142 | 82 | 110 | 111 | 445 |
| Mean (SD) | 72.68 | 75.18 | 69.93 | 77.27 | 73.61 |
| | (21.322) | (21.516) | (16.194) | (24.621) | (21.233) |
| Median | 66.10 | 70.25 | 67.15 | 71.20 | 68.50 |
| Min, Max | 37.5, 138.8 | 40.8, 133.4 | 40.4, 110.7 | 43.5, 163.3 | 37.5, 163.3 |
| Height (cm) | | | | | |
| n | 142 | 82 | 110 | 111 | 445 |
| Mean (SD) | 166.87 | 167.34 | 166.05 | 166.48 | 166.66 |
| | (10.802) | (10.330) | (9.451) | (9.182) | (9.977) |
| Median | 165.10 | 165.10 | 165.00 | 167.60 | 165.10 |
| Min, Max | 129.5, 190.5 | 125.2, 193.0 | 143.0, 188.0 | 124.5, 188.0 | 124.5, 193.0 |
| BMI (kg/m2) | | | | | |
| n | 142 | 82 | 110 | 111 | 445 |
| Mean (SD) | 26.10 (7.429) | 26.81 (7.157) | 25.37 (5.654) | 27.81 (8.259) | 26.48 (7.241) |
| Median | 24.10 | 24.90 | 23.55 | 25.90 | 24.30 |
| Min, Max | 15.6, 54.2 | 17.2, 50.2 | 16.6, 44.6 | 17.6, 59.0 | 15.6, 59.0 |
| Baseline inflammatory | | | | | |
| lesion count | | | | | |
| n | 142 | 82 | 110 | 111 | 445 |
| Mean (SD) | 30.2 (7.61) | 30.1 (8.18) | 31.0 (8.71) | 31.1 (8.62) | 30.6 (8.24) |
| Median | 27.5 | 28.0 | 28.0 | 29.0 | 28.0 |
| Min, Max | 20, 50 | 20, 50 | 20, 50 | 20, 50 | 20, 50 |
| Baseline non-inflammatory | | | | | |
| lesion count | | | | | |
| | 142 | 82 | 110 | 111 | 445 |
| Mean (SD) | 47.2 (16.74) | 49.9 (16.59) | 48.4 (19.08) | 47.7 (16.91) | 48.1 (17.33) |
| Median | 44.5 | 46.0 | 42.5 | 44.0 | 45.0 |
| Min, Max | 25, 94 | 26, 86 | 25, 99 | 25, 98 | 25, 99 |
| Baseline IGA score | | | | | |
| Moderate | 134 (94.4%) | 75 (91.5%) | 96 (87.3%) | 99 (89.2%) | 404 (90.8%) |
| Severe | 8 (5.6%) | 7 (8.5%) | 14 (12.7%) | 12 (10.8%) | 41 (9.2%) |

The mean treatment duration was 83 days and the mean study drug exposure duration was 82 days resulting in a mean study drug compliance of 98.9% in the ITT, Safety and PP populations (See Tables 44A-44C).

TABLE 44A
Study Drug Exposure and Compliance-ITT Population

| | | | Minocycline | Adapalene | |
|---|---|---|---|---|---|
| | FCD105 | Vehicle | 3% | 0.3% | Total |
| | (N = 142) | (N = 83) | (N = 110) | (N = 112) | (N = 447) |
| Treatment duration (days)^{[1]} | | | | | |
| n | 142 | 83 | 110 | 112 | 447 |
| Mean (SD) | 81.4 (16.80) | 83.9 (11.51) | 85.0 (8.07) | 82.8 (12.67) | 83.1 (13.10) |
| Median | 84.0 | 84.0 | 84.0 | 84.0 | 84.0 |
| Min, Max | 5, 122 | 0, 112 | 34, 112 | 15, 123 | 0, 123 |
| Study drug exposure (days)^{[2]} | | | | | |
| n | 142 | 83 | 110 | 112 | 447 |
| Mean (SD) | 80.8 (16.74) | 83.0 (11.38) | 83.6 (8.77) | 81.8 (12.32) | 82.2 (13.05) |
| Median | 84.0 | 84.0 | 84.0 | 84.0 | 84.0 |
| Min, Max | 5, 122 | 0, 112 | 34, 112 | 15, 111 | 0,122 |
| Study Drug Compliance (%)^{[3]} | | | | | |
| n | 140 | 81 | 107 | 108 | 436 |
| Mean (SD) | 99.26 | 98.95 | 98.23 | 99.06 | 98.90 |
| | (1.952) | (2.113) | (3.642) | (1.996) | (2.532) |
| Median | 100.00 | 100.00 | 100.00 | 100.00 | 100.00 |
| Min, Max | 86.2, 100.0 | 89.1, 100.0 | 75.0, 100.0 | 90.2, 100.0 | 75.0, 100.0 |

^{[1]}Treatment duration is defined as the date of last dose of study drug − date of first dose of study drug + 1 day. For subjects who are missing the date of last study drug application, the last known contact date will be used in the calculation of treatment duration and study drug exposure.
^{[2]}Study drug exposure is defined as treatment duration-number of days that a subject reported missing a dose (between the date of first and last dose).
^{[3]}Compliance is defined as 100 × study drug exposure (days)/treatment duration (days). Study drug compliance will not be calculated for subjects whose date of last study drug application is unknown.

TABLE 44B
Study Drug Exposure and Compliance-Safety Population

| | | | Minocycline | Adapalene | |
|---|---|---|---|---|---|
| | FCD105 | Vehicle | 3% | 0.3% | Total |
| | (N = 142) | (N = 83) | (N = 110) | (N = 112) | (N = 447) |
| Treatment duration (days)^{[1]} | | | | | |
| n | 142 | 82 | 110 | 112 | 446 |
| Mean (SD) | 81.4 (16.80) | 85.0 (6.78) | 85.0 (8.07) | 82.8 (12.67) | 83.1 (13.10) |
| Median | 84.0 | 84.0 | 84.0 | 84.0 | 84.0 |
| Min, Max | 5, 122 | 0, 112 | 34, 112 | 15, 123 | 0, 123 |
| Study drug exposure (days)^{[2]} | | | | | |
| n | 142 | 82 | 110 | 112 | 446 |
| Mean (SD) | 80.8 (16.74) | 84.1 (6.70) | 83.6 (8.77) | 81.8 (12.32) | 82.2 (12.47) |
| Median | 84.0 | 84.0 | 84.0 | 84.0 | 84.0 |
| Min, Max | 5, 122 | 50, 112 | 34, 112 | 15, 111 | 5, 122 |
| Study Drug Compliance (%)^{[3]} | | | | | |
| n | 140 | 81 | 107 | 108 | 436 |
| Mean (SD) | 99.26 (1.952) | 98.95 (2.113) | 98.23 (3.642) | 99.06 (1.996) | 98.90 (2.532) |
| Median | 100.00 | 100.00 | 100.00 | 100.00 | 100.00 |
| Min, Max | 86.2, 100.0 | 89.1, 100.0 | 75.0, 100.0 | 90.2, 100.0 | 75.0, 100.0 |

^{[1]}Treatment duration is defined as the date of last dose of study drug − date of first dose of study drug + 1 day. For subjects who are missing the date of last study drug application, the last known contact date will be used in the calculation of treatment duration and study drug exposure.
^{[2]}Study drug exposure is defined as treatment duration-number of days that a subject reported missing a dose (between the date of first and last dose).
^{[3]}Compliance is defined as 100 × study drug exposure (days)/treatment duration (days). Study drug compliance will not be calculated for subjects whose date of last study drug application is unknown.

TABLE 44C
Study Drug Exposure and Compliance-Per-Protocol Population

| | | | Minocycline | Adapalene | |
|---|---|---|---|---|---|
| | FCD105 | Vehicle | 3% | 0.3% | Total |
| | (N = 142) | (N = 82) | (N = 110) | (N = 111) | (N = 445) |
| Treatment duration (days)^{[1]} | | | | | |
| n | 142 | 82 | 110 | 111 | 445 |
| Mean (SD) | 81.4 (16.80) | 85.0 (6.78) | 85.0 (8.07) | 82.7 (12.72) | 83.3 (12.52) |
| Median | 84.0 | 84.0 | 84.0 | 84.0 | 84.0 |
| Min, Max | 5, 122 | 50, 112 | 34, 112 | 15, 123 | 5,123 |
| Study drug exposure (days)^{[2]} | | | | | |
| n | 142 | 82 | 110 | 111 | 445 |
| Mean (SD) | 80.8 (16.74) | 84.1 (6.70) | 83.6 (8.77) | 81.8 (12.36) | 82.3 (12.48) |
| Median | 84.0 | 84.0 | 84.0 | 84.0 | 84.0 |
| Min, Max | 5, 122 | 50, 112 | 34, 112 | 15, 111 | 5, 122 |
| Study Drug Compliance (%)^{[3]} | | | | | |
| n | 140 | 81 | 107 | 107 | 435 |
| Mean (SD) | 99.26 (1.952) | 98.95 (2.113) | 98.23 (3.642) | 99.05 (2.003) | 98.90 (2.534) |
| Median | 100.00 | 100.00 | 100.00 | 100.00 | 100.00 |
| Min, Max | 86.2, 100.0 | 89.1, 100.0 | 75.0, 100.0 | 90.2, 100.0 | 75.0, 100.0 |

^{[1]}Treatment duration is defined as the date of last dose of study drug − date of first dose of study drug + 1 day. For subjects who are missing the date of last study drug application, the last known contact date will be used in the calculation of treatment duration and study drug exposure.
^{[2]}Study drug exposure is defined as treatment duration-number of days that a subject reported missing a dose (between the date of first and last dose).
^{[3]}Compliance is defined as 100 × study drug exposure (days)/treatment duration (days). Study drug compliance will not be calculated for subjects whose date of last study drug application is unknown.

FCD105 was shown to be highly statistically superior to vehicle for the endpoints of (1) Investigator's Global Assessment (IGA) treatment success (IGA score “0” or “1” and two grade reduction in IGA score from baseline) and (2) absolute change from baseline in mean inflammatory counts at Week 12.

The proportion of patients achieving IGA treatment success in the FCD105 treatment group was 35.9% compared to 15.7% of patients in the vehicle treatment group at Week 12 in ITT population (p=0.0003 Multiple Imputation) and (p=0.0006 OC, LOCF and BOCF Imputation Methods) (See Table 45A and Table 45C). Statistical significance in this population was achieved as early as week 8 with 14.8% in the FCD105 treatment group compared to 8.4% of patients in the vehicle treatment group (p=0.0491 Multiple Imputation method) but was only numerically superior at week 4 (Table 45B).

The proportion of patients achieving IGA treatment success at week 12 in the FCD105 treatment group was almost identical in PP population and was 35.9% in the FCD105 treatment group compared to 15.9% of patients in the vehicle treatment group in the (p=0.0001) (See Table 45F and 45G). Statistical significance was demonstrated as early as week 8 and was 14.8% in the FCD105 treatment group compared to 8.5% of patients in the vehicle treatment group (p=0.0345) and numerically superior over vehicle treatment group at Week 4 (see Table 45G).

The proportion of patients in the ITT population achieving secondary endpoint IGA treatment success (e.g., two grade reduction in IGA score from baseline) at Week 12 was also statistically significant in the FCD105 treatment group as compared to vehicle group and was 36.6% compared to 16.9% of patients in the vehicle treatment group (p=0.0004 Multiple Imputation) (Table 45A and Table 45E) and 36.6% compared to 17.1% of patients in the vehicle treatment group in PP population (p=0.0002 Secondary—Multiple Imputation) (Table 45F and Table 451).

The trial was not powered to demonstrate a statistical difference between FCD105 and either 3% minocycline foam or 0.3% adapalene foam treatments, however FCD105 foam was statistically significant over 0.3% adapalene foam (ITT 35.9% vs 21.4% p=0.0114 and 35.9% vs 21.6% PP p=0.013) and numerically superior over 3% minocycline foam at Week 12 in both the ITT and PP populations (see Table 45D and 45H respectively).

Absolute reduction in inflammatory lesion counts at Week 12 was −19.3 (−62.9%) for the FCD105 treatment group compared to −15.4(−.49.3%) for the vehicle treatment group and was statistically significant according to different imputation methods for the ITT Population (MI p=0.0020; OC p=0.0011; LOCF p=0.0334; BOCF p=0.0145; ranked change MI p=0.0052). Statistical significance for the FCD105 treatment group compared to vehicle group at week 12 was also shown by analysis of the percent change from baseline for the ITT population (Tables 46A-D) and PP population (Tables 47A-47C).

The trial was not powered to demonstrate a statistical difference between FCD105 and either 3% minocycline foam or 0.3% adapalene foam treatments, however, a majority of these comparisons were statistically significant at Week 12. Numerical superiority was observed for all efficacy endpoints for these comparisons at Week 12.

Analysis of percent change from baseline inflammatory lesion count —multiple imputation demonstrated that FCD105 foam was statistically significant over 0.3% adapalene foam as early as week 4 (ITT p=0.0179 and PP p=0.0122) and numerically superior over 3% minocycline foam at Week 8 in both the ITT and PP populations (see Table 46E and 47D respectively).

TABLE 45A
Descriptive Summary of IGA Treatment Success-ITT Population

| | | | Minocycline | Adapalene | |
|---|---|---|---|---|---|
| | FCD105 | Vehicle | 3% | 0.3% | Total |
| | (N = 142) | (N = 83) | (N = 110) | (N = 112) | (N = 447) |
| IGA Score | | | | | |
| Baseline | | | | | |
| Moderate | 134 (94.4%) | 76 (91.6%) | 96 (87.3%) | 100 (89.3%) | 406 (90.8%) |
| Severe | 8 (5.6%) | 7 (8.4%) | 14 (12.7%) | 12 (10.7%) | 41 (9.2%) |
| Week 4 | | | | | |
| Clear | 0 | 0 | 0 | 0 | 0 |
| Minimal | 5 (3.5%) | 2 (2.4%) | 2 (1.8%) | 4 (3.6%) | 13 (2.9%) |
| Mild | 53 (37.3%) | 27 (32.5%) | 40 (36.4%) | 29 (25.9%) | 149 (33.3%) |
| Moderate | 72 (50.7%) | 49 (59.0%) | 59 (53.6%) | 67 (59.8%) | 247 (55.3%) |
| Severe | 4 (2.8%) | 4 (4.8%) | 5 (4.5%) | 4 (3.6%) | 17 (3.8%) |
| Week 8 | | | | | |
| Clear | 0 | 0 | 0 | 1 (0.9%) | 1 (0.2%) |
| Minimal | 21 (14.8%) | 7 (8.4%) | 12 (10.9%) | 12 (10.7%) | 52 (11.6%) |
| Mild | 60 (42.3%) | 36 (43.4%) | 47 (42.7%) | 37 (33.0%) | 180 (40.3%) |
| Moderate | 47 (33.1%) | 34 (41.0%) | 41 (37.3%) | 54 (48.2%) | 176 (39.4%) |
| Severe | 0 | 1 (1.2%) | 1 (0.9%) | 1 (0.9%) | 3 (0.7%) |
| Week 12 | | | | | |
| Clear | 4 (2.8%) | 1 (1.2%) | 0 | 1 (0.9%) | 6 (1.3%) |
| Minimal | 47 (33.1%) | 12 (14.5%) | 33 (30.0%) | 23 (20.5%) | 115 (25.7%) |
| Mild | 49 (34.5%) | 39 (47.0%) | 44 (40.0%) | 39 (34.8%) | 171 (38.3%) |
| Moderate | 28 (19.7%) | 22 (26.5%) | 25 (22.7%) | 35 (31.3%) | 110 (24.6%) |
| Severe | 0 | 2 (2.4%) | 2 (1.8%) | 1 (0.9%) | 5 (1.1%) |
| IGA Treatment Success | | | | | |
| Week 4 | | | | | |
| Yes | 5 (3.5%) | 2 (2.4%) | 2 (1.8%) | 4 (3.6%) | 13 (2.9%) |
| No | 129 (90.8%) | 80 (96.4%) | 104 (94.5%) | 100 (89.3%) | 413 (92.4%) |
| Week 8 | | | | | |
| Yes | 21 (14.8%) | 7 (8.4%) | 12 (10.9%) | 13 (11.6%) | 53 (11.9%) |
| No | 107 (75.4%) | 71 (85.5%) | 89 (80.9%) | 92 (82.1%) | 359 (80.3%) |
| Week 12 | | | | | |
| Yes | 51 (35.9%) | 13 (15.7%) | 33 (30.0%) | 24 (21.4%) | 121 (27.1%) |
| No | 77 (54.2%) | 63 (75.9%) | 71 (64.5%) | 75 (67.0%) | 286 (64.0%) |
| IGA Treatment Success | | | | | |
| (Secondary) at Week 12 | | | | | |
| Yes | 52 (36.6%) | 14 (16.9%) | 41 (37.3%) | 29 (25.9%) | 136 (30.4%) |
| No | 76 (53.5%) | 62 (74.7%) | 63 (57.3%) | 70 (62.5%) | 271 (60.6%) |

Note:
IGA Treatment Success is defined as an IGA score of 0 or 1, and at least a 2-grade improvement (decrease) from Baseline.
Note:
IGA Treatment Success (Secondary) is defined as at least a 2-grade improvement (decrease) from Baseline in IGA score.
Note:
IGA Treatment Success is based on observed data.

TABLE 45B
Analysis of IGA Treatment Success-Multiple Imputation (Week 8
and Week 4)-ITT Population

| Visit/ | FCD105 | Vehicle |
|---|---|---|
| Statistics | (N = 142) | (N = 83) |
| Week 8 | | |
| Number of IGA Treatment Successes (n) | 21 | 7 |
| Proportion of IGA Treatment Successes (%) | 14.8% | 8.4% |
| Risk Ratio (SE) | 2.23 (1.55) | |
| 95% CI | 0.94-5.25 | |
| P-value | 0.0491 | |
| Week 4 | | |
| Number of IGA Treatment Successes (n) | 5 | 2 |
| Proportion of IGA Treatment Successes (%) | 3.5% | 2.4% |
| Risk Ratio (SE) | 1.79 (2.27) | |
| 95% CI | 0.36-8.98 | |
| P-value | 0.4770 | |

Note:
IGA Treatment Success is defined as an IGA score of 0 or 1, and at least a 2-grade improvement (decrease) from Baseline.
Note:
Cochran-Mantel-Haenszel test stratified by analysis center. P-value is for the null hypothesis that the Risk Ratio equals 1.

TABLE 45C
Analysis of IGA Treatment Success at Week 12-by Different
Imputations-ITT Population

| Imputation | | FCD105 | Vehicle |
|---|---|---|---|
| Method | Statistics | (N = 142) | (N = 83) |
| OC | Number of IGA Treatment Successes (n) | 51 | 13 |
| | Proportion of IGA Treatment Successes | 35.9% | 15.7% |
| | (%) | | |
| | Risk Ratio (SE) | 2.47 (1.34) | |
| | 95% CI | 1.40-4.36 | |
| | P-value | 0.0006 | |
| LOCF | Number of IGA Treatment Successes (n) | 51 | 13 |
| | Proportion of IGA Treatment Successes | 35.9% | 15.7% |
| | (%) | | |
| | Risk Ratio (SE) | 2.47 (1.34) | |
| | 95% CI | 1.40-4.36 | |
| | P-value | 0.0006 | |
| BOCF | Number of IGA Treatment Successes (n) | 51 | 13 |
| | Proportion of IGA Treatment Successes | 35.9% | 15.7% |
| | (%) | | |
| | Risk Ratio (SE) | 2.47 (1.34) | |
| | 95% CI | 1.40-4.36 | |
| | P-value | 0.0006 | |
| MI | Number of IGA Treatment Successes (n) | 51 | 13 |
| | Proportion of IGA Treatment Successes | 35.9% | 15.7% |
| | (%) | | |
| | Risk Ratio (SE) | 2.63 (1.34) | |
| | 95% CI | 1.48-4.66 | |
| | P-value | 0.0003 | |

Note:
IGA Treatment Success is defined as an IGA score of 0 or 1, and at least a 2-grade improvement (decrease) from Baseline.
Note:
Cochran-Mantel-Haenszel test stratified by analysis center. P-value is for the null hypothesis that the Risk Ratio equals 1.
Note:
OC = Observed Cases;
LOCF = Last Observation Carried Forward;
BOCF = Baseline Observation Carried Forward;
MI = Multiple Imputation.

TABLE 45D
Analysis of IGA Treatment Success at Week 12-Multiple
Imputation-ITT Population

| | FCD105 | Minocycline 3% | Adapalene 0.3% |
|---|---|---|---|
| Statistics | (N = 142) | (N = 110) | (N = 112) |
| Number of IGA Treatment Successes (n) | 51 | 33 | 24 |
| Proportion of IGA Treatment Successes (%) | 35.9% | 30.0% | 21.4% |
| Risk Ratio (SE) | | 0.82 (1.19) | 0.60 (1.23) |
| 95% CI | | 0.58-1.15 | 0.40-0.90 |
| P-value | | 0.2569 | 0.0114 |

Note:
IGA Treatment Success is defined as an IGA score of 0 or 1, and at least a 2-grade improvement (decrease) from Baseline.
Note:
Cochran-Mantel-Haenszel test stratified by analysis center. P-value is for the null hypothesis that the Risk Ratio equals 1.

TABLE 45E
Analysis of IGA Treatment Success at Week 12-Secondary-
Multiple Imputation-ITT Population

| | FCD105 | Vehicle |
|---|---|---|
| Statistics | (N = 142) | (N = 83) |
| Number of IGA Treatment Successes (n) | 52 | 14 |
| Proportion of IGA Treatment Successes (%) | 36.6% | 16.9% |
| Risk Ratio (SE) | 2.46 (1.32) | |
| 95% CI | 1.42-4.25 | |
| P-value | 0.0004 | |

Note:
IGA Treatment Success (Secondary) is defined as at least a 2-grade improvement (decrease) from Baseline in IGA score.
Note:
Cochran-Mantel-Haenszel test stratified by analysis center. P-value is for the null hypothesis that the Risk Ratio equals 1.

TABLE 45F
Descriptive Summary of IGA Treatment Success-Per-Protocol
Population

| | | | Minocycline | Adapalene | |
|---|---|---|---|---|---|
| | FCD105 | Vehicle | 3% | 0.3% | Total |
| | (N = 142) | (N = 82) | (N = 110) | (N = 111) | (N = 445) |
| IGA Score | | | | | |
| Baseline | | | | | |
| Moderate | 134 (94.4%) | 75 (91.5%) | 96 (87.3%) | 99 (89.2%) | 404 (90.8%) |
| Severe | 8 (5.6%) | 7 (8.5%) | 14 (12.7%) | 12 (10.8%) | 41 (9.2%) |
| Week 4 | | | | | |
| Clear | 0 | 0 | 0 | 0 | 0 |
| Minimal | 5 (3.5%) | 2 (2.4%) | 2 (1.8%) | 4 (3.6%) | 13 (2.9%) |
| Mild | 53 (37.3%) | 27 (32.9%) | 40 (36.4%) | 29 (26.1%) | 149 (33.5%) |
| Moderate | 72 (50.7%) | 49 (59.8%) | 59 (53.6%) | 66 (59.5%) | 246 (55.3%) |
| Severe | 4 (2.8%) | 4 (4.9%) | 5 (4.5%) | 4 (3.6%) | 17 (3.8%) |
| Week 8 | | | | | |
| Clear | 0 | 0 | 0 | 1 (0.9%) | 1 (0.2%) |
| Minimal | 21 (14.8%) | 7 (8.5%) | 12 (10.9%) | 12 (10.8%) | 52 (11.7%) |
| Mild | 60 (42.3%) | 36 (43.9%) | 47 (42.7%) | 36 (32.4%) | 179 (40,2%) |
| Moderate | 47 (33.1%) | 34 (41.5%) | 41 (37.3%) | 54 (48.6%) | 176 (39.6%) |
| Severe | 0 | 1 (1.2%) | 1 (0.9%) | 1 (0.9%) | 3 (0.7%) |
| Week 12 | | | | | |
| Clear | 1 (2.8%) | 1 (1.2%) | 0 | 1 (0.9%) | 6 (1.3%) |
| Minimal | 47 (33.1%) | 12 (14.6%) | 33 (30.0%) | 23 (20.7%) | 115 (25.8%) |
| Mild | 49 (34.5%) | 39 (47.6%) | 44 (40.0%) | 38 (34.2%) | 170 (38.2%) |
| Moderate | 28 (19.7%) | 22 (26.8%) | 25 (22.7%) | 35 (31.5%) | 110 (24,7%) |
| Severe | 0 | 2 (2.4%) | 2 (1.8%) | 1 (0.9%) | 5 (1.1%) |
| IGA Treatment Success | | | | | |
| Week 4 | | | | | |
| Yes | 5 (3.5%) | 2 (2.4%) | 2 (1.8%) | 4 (3.6%) | 13 (2.9%) |
| No | 129 (90.8%) | 80 (97.6%) | 104 (94.5%) | 99 (89.2%) | 412 (92.6%) |
| Week 8 | | | | | |
| Yes | 21 (14.8%) | 7 (8.5%) | 12 (10.9%) | 13 (11.7%) | 53 (11.9%) |
| No | 107 (75.4%) | 71 (86.6%) | 89 (80.9%) | 91 (82.0%) | 358 (80.4%) |
| Week 12 | | | | | |
| Yes | 51 (35.9%) | 13 (15.9%) | 33 (30.0%) | 24 (21.6%) | 121 (27.2%) |
| No | 77 (54.2%) | 63 (76.8%) | 71 (64.5%) | 74 (66.7%) | 285 (64.0%) |
| IGA Treatment Success | | | | | |
| (Secondary) at Week 12 | | | | | |
| Yes | 52 (36.6%) | 14 (17.1%) | 41 (37.3%) | 29 (26.1%) | 136 (30.6%) |
| No | 76 (53.5%) | 62 (75.6%) | 63 (57.3%) | 69 (62.2%) | 270 (60.7%) |

Note:
IGA Treatment Success is defined as an IGA score of 0 or 1, and at least a 2-grade improvement (decrease) from Baseline.
Note:
IGA Treatment Success (Secondary) is defined as at least a 2-grade improvement (decrease) from Baseline in IGA score.
Note:
IGA Treatment Success is based on observed data.

TABLE 45G
Analysis of IGA Treatment Success at (Week 12,
Week 8 and Week 4)-Per-Protocol Population

| Visit/ | FCD105 | Vehicle |
|---|---|---|
| Statistics | (N = 142) | (N = 82) |
| Week 12 | | |
| Number of IGA Treatment Successes (n) | 51 | 13 |
| Proportion of IGA Treatment Successes (%) | 35.9% | 15.9% |
| Risk Ratio (SE) | 2.73 (1.34) | |
| 95% CI | 1.54-4.86 | |
| P-value | 0.0001 | |
| Week 8 | | |
| Number of IGA Treatment Successes (n) | 21 | 7 |
| Proportion of IGA Treatment Successes (%) | 14.8% | 8.5% |
| Risk Ratio (SE) | 2.42 (1.56) | |
| 95% CI | 1.01-5.75 | |
| P-value | 0.0345 | |
| Week 4 | | |
| Number of IGA Treatment Successes (n) | 5 | 2 |
| Proportion of IGA Treatment Successes (%) | 3.5% | 2.4% |
| Risk Ratio (SE) | 1.79 (2.27) | |
| 95% CI | 0.36-8.98 | |
| P-value | 0.4770 | |

Note:
IGA Treatment Success is defined as an IGA score of 0 or 1, and at least a 2-grade improvement (decrease) from Baseline.
Note:
Cochran-Mantel-Haenszel test stratified by analysis center. P-value is for the null hypothesis that the Risk Ratio equals 1.

TABLE 45H
Analysis of IGA Treatment Success at Week 12-Multiple
Imputation-Per-Protocol Population

| | FCD105 | Minocycline 3% | Adapalene 0.3% |
|---|---|---|---|
| Statistics | (N = 142) | (N = 110) | (N = 111) |
| Number of IGA Treatment Successes (n) | 51 | 33 | 24 |
| Proportion of IGA Treatment Successes (%) | 35.9% | 30.0% | 21.6% |
| Risk Ratio (SE) | | 0.81 (1.19) | 0.61 (1.23) |
| 95% CI | | 0.58-1.15 | 0.40-0.91 |
| P-value | | 0.2438 | 0.0130 |

Note:
IGA Treatment Success is defined as an IGA score of 0 or 1, and at least a 2-grade improvement (decrease) from Baseline.
Note:
Cochran-Mantel-Haenszel test stratified by analysis center. P-value is for the null hypothesis that the Risk Ratio equals 1.

TABLE 45I
Analysis of IGA Treatment Success at Week 12-Secondary-
Multiple Imputation-Per-Protocol Population

| | FCD105 | Vehicle |
|---|---|---|
| Statistics | (N = 142) | (N = 82) |
| Number of IGA Treatment Successes (n) | 52 | 14 |
| Proportion of IGA Treatment Successes (%) | 36.6% | 17.1% |
| Risk Ratio (SE) | 2.56 (1.32) | |
| 95% CI | 1.48-4.41 | |
| P-value | 0.0002 | |

Note:
IGA Treatment Success (Secondary) is defined as at least a 2-grade improvement (decrease) from Baseline in IGA score.
Note:
Cochran-Mantel-Haenszel test stratified by analysis center. P-value is for the null hypothesis that the Risk Ratio equals 1.

Absolute reduction in non-inflammatory lesion counts at Week 12 was also assessed with a mean lesion count reduction of 25.9 (−53.83%) for the FCD105 treatment group compared to 24.1 (−48.09%) for the vehicle treatment group. Although numerically superior this was not statistically significant, which may be attributed to outlier results affecting both FCD105 and vehicle treatment groups. Analysis of the absolute count reduction and percent change from baseline in non-inflammatory lesion count also showed numerical superiority, although this comparison was also not statistically significant (See Table 48A-48D for ITT population and Table 49A-49C for PP population). Conversely, absolute reduction in non-inflammatory lesion counts at Week 12 for FCD105 compared to either of the active comparator groups was statistically superior to each of (1) 3% minocycline foam and (2) 0.3% adapalene foam (Table 48E for ITT population and Table 49D for PP population). A reduction in non-inflammatory lesion counts was observed at week 4 and appeared to achieve a peak effect by about week 8, as a similar reduction was observed at week 12.

TABLE 46A
Descriptive Summary of Inflammatory Lesion Count-ITT
Population

| | | FCD105 | Vehicle | Minocycline 3% | Adapalene 0.3% | Total |
|---|---|---|---|---|---|---|
| Visit | Statistics | (N = 142) | (N = 83) | (N = 110) | (N = 112) | (N = 447) |
| Baseline | n | 142 | 83 | 110 | 112 | 447 |
| | Mean (SD) | 30.2 (7.61) | 30.0 (8.14) | 31.0 (8.71) | 31.1 (8.58) | 30.6 (8.22) |
| | Median | 27.5 | 28.0 | 28.0 | 29.0 | 28.0 |
| | Min, Max | 20, 50 | 20, 50 | 20, 50 | 20, 50 | 20, 50 |
| Week 4 | n | 134 | 82 | 106 | 104 | 426 |
| | Mean (SD) | 20.6 (9.53) | 22.4 (10.78) | 20.3 (9.95) | 23.7 (10.37) | 21.6 (10.15) |
| | Median | 20.0 | 21.0 | 19.5 | 23.0 | 21.0 |
| | Min, Max | 4, 54 | 2, 59 | 2, 41 | 3, 49 | 2, 59 |
| | Change from | | | | | |
| | Baseline | | | | | |
| | n | 134 | 82 | 106 | 104 | 426 |
| | Mean (SD) | 9.6 (8.88) | 7.7 (9.12) | 10.6 (9.30) | 7.5 (8.83) | 8.9 (9.08) |
| | Median | 10.5 | 7.0 | 10.5 | 6.5 | 9.0 |
| | Min, Max | −27, 30 | −19, 27 | −12, 36 | −25, 32 | −27, 36 |
| | Percent Change | | | | | |
| | from Baseline | | | | | |
| | n | 134 | 82 | 106 | 104 | 426 |
| | Mean (SD) | 30.94 (29.765) | 25.55 (29.181) | 33.69 (29.987) | 23.21 (30.333) | 28.70 (30.029) |
| | Median | 30.60 | 22.41 | 31.24 | 21.39 | 27.27 |
| | Min, Max | −112.5, 87.9 | −48.4, 91.3 | −52.2, 91.3 | −125.0, 86.2 | −125.0, 91.3 |
| Week 8 | n | 128 | 78 | 101 | 104 | 411 |
| | Mean (SD) | 15.4 (8.69) | 16.5 (10.71) | 16.6 (9.41) | 19.2 (10.50) | 16.9 (9.82) |
| | Median | 14.0 | 15.0 | 15.0 | 19.0 | 15.0 |
| | Min, Max | 0, 47 | 1, 63 | 0, 42 | 0, 51 | 0, 63 |
| | Change from | | | | | |
| | Baseline | | | | | |
| | n | 128 | 78 | 101 | 104 | 411 |
| | Mean (SD) | 14.7 (9.32) | 13.4 (10.58) | 14.4 (10.03) | 12.2 (10.54) | 13.7 (10.07) |
| | Median | 16.0 | 14.0 | 15.0 | 13.0 | 14.0 |
| | Min, Max | −14, 34 | −36, 35 | −16, 39 | −27, 38 | −36, 39 |
| | Percent Change | | | | | |
| | from Baseline | | | | | |
| | n | 128 | 78 | 101 | 104 | 411 |
| | Mean (SD) | 47.99 (29.055) | 44.58 (32.403) | 45.54 (30.889) | 37.65 (34.786) | 44.12 (31.800) |
| | Median | 52.17 | 46.83 | 52.17 | 39.23 | 48.94 |
| | Min, Max | −45.8, 100.0 | −133.3, 95.5 | −61.5, 100.0 | −135.0, 100.0 | −135.0, 100.0 |
| Week 12 | n | 128 | 76 | 104 | 99 | 407 |
| | Mean (SD) | 11.1 (8.64) | 15.0 (10.08) | 12.7 (8.74) | 15.7 (11.33) | 13.4 (9.80) |
| | Median | 9.0 | 13.0 | 12.0 | 13.0 | 11.0 |
| | Min, Max | 0, 43 | 0, 55 | 0, 47 | 1, 49 | 0, 55 |
| | Change from | | | | | |
| | Baseline | | | | | |
| | n | 128 | 76 | 104 | 99 | 407 |
| | Mean (SD) | 19.3 (9.71) | 15.4 (11.31) | 18.3 (10.84) | 15.6 (11.31) | 17.4 (10.80) |
| | Median | 19.5 | 17.0 | 19.5 | 17.0 | 18.0 |
| | Min, Max | −10, 44 | −15, 37 | −21, 46 | −15, 37 | −21, 46 |
| | Percent Change | | | | | |
| | from Baseline | | | | | |
| | n | 128 | 76 | 104 | 99 | 407 |
| | Mean (SD) | 62.95 (27.859) | 49.28 (32.518) | 57.41 (30.887) | 49.48 (34.522) | 55.71 (31.659) |
| | Median | 69.57 | 57.84 | 66.29 | 60.00 | 62.96 |
| | Min, Max | −47.6, 100.0 | −37.5, 100.0 | −80.8, 100.0 | −60.0, 97.1 | −80.8, 100.0 |

Note:
Changes from baseline lesion counts are calculated as the baseline value minus the post-baseline value.
Note:
Percent changes from baseline lesion count are calculated as the baseline value minus the post-baseline value divided by the baseline value, expressed as a percentage.
Note:
OC = Observed Cases;
LOCF = Last Observation Carried Forward;
BOCF = Baseline Observation Carried Forward.

TABLE 46B
Analysis of Change from Baseline In Inflammatory Lesion Count At
Week 12-by Different Imputation Methods of ITT Population

| Imputation | | FCD105 | Vehicle |
|---|---|---|---|
| Method | Statistics | (N = 142) | (N = 83) |
| OC | LSMean ^{[1]} | 19.79 | 15.65 |
| | Std Err LSMean | 0.93 | 1.10 |
| | 95% CI of LSMean | 17.95-21.63 | 13.48-17.81 |
| | LSMean Difference | 4.14 | |
| | Std Err Difference | 1.25 | |
| | 95% CI of LSMean Difference | 1.68-6.61 | |
| | P-value ^{[2]} | 0.0011 | |
| LOCF | LSMean ^{[1]} | 18.16 | 15.37 |
| | Std Err LSMean | 0.97 | 1.16 |
| | 95% CI of LSMean | 16.24-20.07 | 13.09-17.65 |
| | LSMean Difference | 2.79 | |
| | Std Err Difference | 1.30 | |
| | 95% CI of LSMean Difference | 0.22-5.35 | |
| | P-value ^{[2]} | 0.0334 | |
| BOCF | LSMean ^{[1]} | 17.93 | 14.56 |
| | Std Err LSMean | 1.00 | 1.20 |
| | 95% CI of LSMean | 15.96-19.90 | 12.20-16.93 |
| | LSMean Difference | 3.37 | |
| | Std Err Difference | 1.36 | |
| | 95% CI of LSMean Difference | 0.68-6.06 | |
| | P-value ^{[2]} | 0.0145 | |
| MI | LSMean ^{[1]} | 19.40 | 15.58 |
| | Std Err LSMean | 0.92 | 1.09 |
| | 95% CI of LSMean | 17.60-21.21 | 13.44-17.72 |
| | LSMean Difference | 3.82 | |
| | Std Err Difference | 1.24 | |
| | 95% CI of LSMean Difference | 1.40-6.25 | |
| | P-value ^{[2]} | 0.0020 | |

Note:
Changes from baseline lesion counts are calculated as the baseline value minus the post-baseline value.
^{[1]} Least Squares Means are defined as model-based linear combination (sum) of the estimated effects.
^{[2]} P-value obtains from ANCOVA repeated measures model with treatment and visit as main effect, baseline inflammatory lesion count as a covariate, and analysis center as a blocking factor.
Note:
OC = Observed Cases;
LOCF = Last Observation Carried Forward;
BOCF = Baseline Observation Carried Forward;
MI = Multiple Imputation.

TABLE 46C
Analysis of Ranked Change from Baseline in Inflammatory Lesion
Count at Week 12-Multiple Imputation-ITT Population

| | FCD105 | Vehicle |
|---|---|---|
| Statistics | (N = 142) | (N = 83) |
| LSMean ^{[1]} | 102.75 | 124.06 |
| Std Err LSMean | 5.72 | 6.80 |
| 95% CI of LSMean | 91.53-113.96 | 110.73-137.40 |
| LSMean Difference | −21.31 | |
| Std Err Difference | 7.63 | |
| 95% CI of LSMean Difference | −36.26-−6.37 | |
| P-value ^{[2]} | 0.0052 | |

Note:
Changes from baseline lesion counts are calculated as the baseline value minus the post-baseline value.
Note:
Least Squares Means are based on rank-transform values.
^{[1]} Least Squares Means are defined as model-based linear combination (sum) of the estimated effects.
^{[2]} P-value obtains from ANCOVA repeated measures model with treatment and visit as main effect, baseline inflammatory lesion count as a covariate, and analysis center as a blocking factor.

TABLE 46D
Analysis of Percent Change from Baseline in Inflammatory Lesion
Count-Multiple Imputation (Week 12, Week 8 and Week 4)-ITT Population

| | FCD105 | Vehicle |
|---|---|---|
| Statistics | (N = 142) | (N = 83) |
| Week 12 | | |
| LSMean ^{[1]} | 64.11 | 50.87 |
| Std Err LSMean | 3.05 | 3.63 |
| 95% CI of LSMean | 58.13-70.08 | 43.76-57.98 |
| LSMean Difference | 13.23 | |
| Std Err Difference | 4.12 | |
| 95% CI of LSMean Difference | 5.16-21.31 | |
| P-value ^{[2]} | 0.0013 | |
| Week 8 | | |
| LSMean ^{[1]} | 50.02 | 46.66 |
| Std Err LSMean | 3.09 | 3.66 |
| 95% CI of LSMean | 43.96-56.08 | 39.49-53.83 |
| LSMean Difference | 3.36 | |
| Std Err Difference | 4.13 | |
| 95% CI of LSMean Difference | −4.74-11.46 | |
| P-value ^{[2]} | 0.4161 | |
| Week 4 | | |
| LSMean ^{[1]} | 32.88 | 27.29 |
| Std Err LSMean | 2.98 | 3.46 |
| 95% CI of LSMean | 27.05-38.71 | 20.52-34.07 |
| LSMean Difference | 5.58 | |
| Std Err Difference | 3.87 | |
| 95% CI of LSMean Difference | −1.99-13.16 | |
| P-value ^{[2]} | 0.1486 | |

Note:
Percent changes from baseline lesion count are calculated as the baseline value minus the post-baseline value divided by the baseline value, expressed as a percentage.
Note:
OC = Observed Cases;
LOCF = Last Observation Carried Forward;
BOCF = Baseline Observation Carried Forward.
^{[1]} Least Squares Means are defined as model-based linear combination (sum) of the estimated effects.
^{[2]} P-value obtains from ANCOVA repeated measures model with treatment and visit as main effect, baseline inflammatory lesion count as a covariate, and analysis center as a blocking factor.

TABLE 46E
Analysis of Change from Baseline in Inflammatory Lesion Count-
Secondary-Multiple Imputation-ITT Population

| | FCD105 | Minocycline 3% | Adapalene 0.3% |
|---|---|---|---|
| Statistics | (N = 142) | (N = 110) | (N = 112) |
| Week 12 | | | |
| LSMean ^{[1]} | 19.66 | 18.67 | 15.78 |
| Std Err LSMean | 0.90 | 0.96 | 0.97 |
| 95% CI of LSMean | 17.90-21.43 | 16.80-20.55 | 13.87-17.69 |
| LSMean Difference | | −0.99 | −3.89 |
| Std Err Difference | | 1.19 | 1.20 |
| 95% CI of LSMean | | −3.32-1.34 | −6.24-−1.53 |
| Difference | | | |
| P-value ^{[2]} | | 0.4053 | 0.0012 |
| Week 8 | | | |
| LSMean ^{[1]} | 15.54 | 14.69 | 12.30 |
| Std Err LSMean | 0.84 | 0.88 | 0.90 |
| 95% CI of LSMean | 13.90-17.18 | 12.97-16.42 | 10.54-14.06 |
| LSMean Difference | | −0.85 | −3.24 |
| Std Err Difference | | 1.08 | 1.10 |
| 95% CI of LSMean | | −2.97-1.28 | −5.40-−1.09 |
| Difference | | | |
| P-value ^{[2]} | | 0.4358 | 0.0032 |
| Week 4 | | | |
| LSMean ^{[1]} | 10.28 | 10.96 | 7.79 |
| Std Err LSMean | 0.81 | 0.86 | 0.89 |
| 95% CI of LSMean | 8.69-11.87 | 9.28-12.64 | 6.05-9.52 |
| LSMean Difference | | 0.68 | −2.49 |
| Std Err Difference | | 1.04 | 1.05 |
| 95% CI of LSMean | | −1.36-2.73 | −4.56-−0.43 |
| Difference | | | |
| P-value ^{[2]} | | 0.5124 | 0.0179 |

Note:
Changes from baseline lesion counts are calculated as the baseline value minus the post-baseline value.
^{[1]} Least Squares Means are defined as model-based linear combination (sum) of the estimated effects.
^{[2]} P-value obtains from ANCOVA repeated measures model with treatment and visit as main effect, baseline inflammatory lesion count as a covariate, and analysis center as a blocking factor.

TABLE 47A
Descriptive Summary of Inflammatory Lesion Count-Per-Protocol
Population

| | | FCD105 | Vehicle | Minocycline | Adapalene | Total |
|---|---|---|---|---|---|---|
| Visit | Statistics | (N = 142) | (N = 82) | 3% (N = 110) | 0.3% (N = 111) | (N = 445) |
| Baseline | n | 142 | 82 | 110 | 111 | 445 |
| | Mean (SD) | 30.2 (7.61) | 30.1 (8.18) | 31.0 (8.71) | 31.1 (8.62) | 30.6 (8.24) |
| | Median | 27.5 | 28.0 | 28.0 | 29.0 | 28.0 |
| | Min, Max | 20, 50 | 20, 50 | 20, 50 | 20, 50 | 20, 50 |
| Week 4 | n | 134 | 82 | 106 | 103 | 425 |
| | Mean (SD) | 20.6 (9.53) | 22.4 (10.78) | 20.3 (9.95) | 23.7 (10.42) | 21.6 (10.16) |
| | Median | 20.0 | 21.0 | 19.5 | 23.0 | 21.0 |
| | Min, Max | 4, 54 | 2, 59 | 2, 41 | 3, 49 | 2, 59 |
| | Change from | | | | | |
| | Baseline | | | | | |
| | n | 134 | 82 | 106 | 103 | 425 |
| | Mean (SD) | 9.6 (8.88) | 7.7 (9.12) | 10.6 (9.30) | 7.4 (8.86) | 8.9 (9.09) |
| | Median | 10.5 | 7.0 | 10.5 | 6.0 | 9.0 |
| | Min, Max | −27, 30 | −19, 27 | −12, 36 | −25, 32 | −27,36 |
| | Percent Change | | | | | |
| | from Baseline | | | | | |
| | n | 134 | 82 | 106 | 103 | 425 |
| | Mean (SD) | 30.94 (29.765) | 25.55 (29.181) | 33.69 (29.987) | 23.05 (30.438) | 28.67 (30.060) |
| | Median | 30.60 | 22.41 | 31.24 | 20.83 | 27.27 |
| | Min, Max | −112.5, 87.9 | −48.4, 91.3 | −52.2, 91.3 | −125.0, 86.2 | −125.0, 91.3 |
| Week 8 | n | 128 | 78 | 101 | 103 | 410 |
| | Mean (SD) | 15.4 (8.69) | 16.5 (10.71) | 16.6 (9.41) | 19.3 (10.54) | 16.9 (9.83) |
| | Median | 14.0 | 15.0 | 15.0 | 19.0 | 15.5 |
| | Min, Max | 0, 47 | 1, 63 | 0, 42 | 0, 51 | 0, 63 |
| | Change from | | | | | |
| | Baseline | | | | | |
| | n | 128 | 78 | 101 | 103 | 410 |
| | Mean (SD) | 14.7 (9.32) | 13.4 (10.58) | 14.4 (10.03) | 12.1 (10.56) | 13.7 (10.08) |
| | Median | 16.0 | 14.0 | 15.0 | 13.0 | 14.0 |
| | Min, Max | −14, 34 | −36, 35 | −16, 39 | −27, 38 | −36, 39 |
| | Percent Change | | | | | |
| | from Baseline | | | | | |
| | n | 128 | 78 | 101 | 103 | 410 |
| | Mean (SD) | 47.99 (29.055) | 44.58 (32.403) | 45.54 (30.889) | 37.42 (34.882) | 44.08 (31.828) |
| | Median | 52.17 | 46.83 | 52.17 | 38.46 | 48.83 |
| | Min, Max | −45.8, 100.0 | −133.3, 95.5 | −61.5, 100.0 | −135.0, 100.0 | −135.0, 100.0 |
| Week 12 | n | 128 | 76 | 104 | 98 | 406 |
| | Mean (SD) | 11.1 (8.64) | 15.0 (10.08) | 12.7 (8.74) | 15.8 (11.38) | 13.4 (9.81) |
| | Median | 9.0 | 13.0 | 12.0 | 13.0 | 11.0 |
| | Min, Max | 0, 43 | 0, 55 | 0, 47 | 1,49 | 0, 55 |
| | Change from | | | | | |
| | Baseline | | | | | |
| | n | 128 | 76 | 104 | 98 | 406 |
| | Mean (SD) | 19.3 (9.71) | 15.4 (11.31) | 18.3 (10.84) | 15.6 (11.36) | 17.4 (10.82) |
| | Median | 19.5 | 17.0 | 19.5 | 17.0 | 18.0 |
| | Min, Max | −10, 44 | −15, 37 | −21, 46 | −15, 37 | −21, 46 |
| | Percent Change | | | | | |
| | from Baseline | | | | | |
| | n | 128 | 76 | 104 | 98 | 406 |
| | Mean (SD) | 62.95 (27.859) | 49.28 (32.518) | 57.41 (30.887) | 49.34 (34.670) | 55.69 (31.695) |
| | Median | 69.57 | 57.84 | 66.29 | 59.55 | 62.88 |
| | Min, Max | −47.6, 100.0 | −37.5, 100.0 | −80.8, 100.0 | 60.0, 97.1 | −80.8, 100.0 |

Note:
Changes from baseline lesion counts are calculated as the baseline value minus the post-baseline value.
Note:
Percent changes from baseline lesion count are calculated as the baseline value minus the post-baseline value divided by the baseline value, expressed as a percentage.
Note:
OC = Observed Cases;
LOCF = Last Observation Carried Forward;
BOCF = Baseline Observation Carried Forward.

TABLE 47B
Analysis of Change from Baseline In Inflammatory Lesion Count at
Week 12-Per-Protocol Population

| | FCD105 | Vehicle |
|---|---|---|
| Statistics | (N = 142) | (N = 82) |
| LSMean ^{[1]} | 19.79 | 15.65 |
| Std Err LSMean | 0.93 | 1.10 |
| 95% CI of LSMean | 17.95-21.63 | 13.48-17.81 |
| LSMean Difference | 4.14 | |
| Std Err Difference | 1.25 | |
| 95% CI of LSMean Difference | 1.68-6.61 | |
| P-value ^{[2]} | 0.0011 | |

Note:
Changes from baseline lesion counts are calculated as the baseline value minus the post-baseline value.
^{[1]} Least Squares Means are defined as model-based linear combination (sum) of the estimated effects.
^{[2]} P-value obtains from ANCOVA repeated measures model with treatment and visit as main effect, baseline inflammatory lesion count as a covariate, and analysis center as a blocking factor.

TABLE 47C
Analysis of Percent Change from Baseline in Inflammatory Lesion
Count (Week 12, Week 8 and Week 4)-Per-Protocol Population

| | FCD105 | Vehicle |
|---|---|---|
| Statistics | (N = 142) | (N = 82) |
| Week 12 | | |
| LSMean ^{[1]} | 65.22 | 51.02 |
| Std Err LSMean | 3.05 | 3.58 |
| 95% CI of LSMean | 59.21-71.24 | 43.96-58.08 |
| LSMean Difference | 14.21 | |
| Std Err Difference | 4.05 | |
| 95% CI of LSMean Difference | 6.20-22.21 | |
| P-value ^{[2]} | 0.0006 | |
| Week 8 | | |
| LSMean ^{[1]} | 50.96 | 46.71 |
| Std Err LSMean | 3.10 | 3.63 |
| 95% CI of LSMean | 44.85-57.07 | 39.56-53.86 |
| LSMean Difference | 4.25 | |
| Std Err Difference | 4.12 | |
| 95% CI of LSMean Difference | −3.87-12.37 | |
| P-value ^{[2]} | 0.3031 | |
| Week 4 | | |
| LSMean ^{[1]} | 33.54 | 27.11 |
| Std Err LSMean | 2.97 | 3.44 |
| 95% CI of LSMean | 27.69-39.39 | 20.33-33.89 |
| LSMean Difference | 6.43 | |
| Std Err Difference | 3.84 | |
| 95% CI of LSMean Difference | −1.15-14.02 | |
| P-value ^{[2]} | 0.0960 | |

Note:
Percent changes from baseline lesion count are calculated as the baseline value minus the post-baseline value divided by the baseline value, expressed as a percentage.
Note:
OC = Observed Cases;
LOCF = Last Observation Carried Forward;
BOCF = Baseline Observation Carried Forward.
^{[1]} Least Squares Means are defined as model-based linear combination (sum) of the estimated effects.
^{[2]} P-value obtains from ANCOVA repeated measures model with treatment and visit as main effect, baseline inflammatory lesion count as a covariate, and analysis center as a blocking factor.

TABLE 47D
Analysis of Change from Baseline in Inflammatory Lesion Count -
Secondary-Multiple Imputation-Per-Protocol Population

| | FCD105 | Minocycline 3% | Adapalene 0.3% |
|---|---|---|---|
| Statistics | (N = 142) | (N = 110) | (N = 111) |
| Week 12 | | | |
| LSMean ^{[1]} | 20.05 | 18.88 | 16.02 |
| Std Err LSMean | 0.92 | 0.97 | 1.00 |
| 95% CI of LSMean | 18.24-21.85 | 16.97-20.80 | 14.06-17.99 |
| LSMean Difference | | −1.16 | −4.03 |
| Std Err Difference | | 1.22 | 1.23 |
| 95% CI of LSMean | | −3.56-1.23 | −6.45-−1.60 |
| Difference | | | |
| P-value ^{[2]} | | 0.3409 | 0.0012 |
| Week 8 | | | |
| LSMean ^{[1]} | 15.83 | 14.89 | 12.46 |
| Std Err LSMean | 0.84 | 0.89 | 0.90 |
| 95% CI of LSMean | 14.17-17.49 | 13.14-16.64 | 10.69-14.23 |
| LSMean Difference | | −0.94 | −3.36 |
| Std Err Difference | | 1.09 | 1.09 |
| 95% CI of LSMean | | −3.09-1.21 | −5.52-−1.21 |
| Difference | | | |
| P-value ^{[2]} | | 0.3912 | 0.0023 |
| Week 4 | | | |
| LSMean ^{[1]} | 10.54 | 11.14 | 7.90 |
| Std Err LSMean | 0.81 | 0.86 | 0.88 |
| 95% CI of LSMean | 8.94-12.14 | 9.45-12.83 | 6.17-9.63 |
| LSMean Difference | | 0.60 | −2.64 |
| Std Err Difference | | 1.04 | 1.05 |
| 95% CI of LSMean | | −1.45-2.64 | −4.70-−0.58 |
| Difference | | | |
| P-value ^{[2]} | | 0.5647 | 0.0122 |

Note:
Changes from baseline lesion counts are calculated as the baseline value minus the post-baseline value.
^{[1]} Least Squares Means are defined as model-based linear combination (sum) of the estimated effects.
^{[2]} P-value obtains from ANCOVA repeated measures model with treatment and visit as main effect, baseline inflammatory lesion count as a covariate, and analysis center as a blocking factor.

TABLE 48A
Descriptive Summary of Non-Inflammatory Lesion Count-ITT
Population

| | | FCD105 | Vehicle | Minocycline 3% | Adapalene 0.3% | Total |
|---|---|---|---|---|---|---|
| Visit | Statistics | (N = 142) | (N = 83) | (N = 110) | (N = 112) | (N = 447) |
| Baseline | n | 142 | 83 | 110 | 112 | 447 |
| | Mean (SD) | 47.2 (16.74) | 49.8 (16.51) | 48.4 (19.08) | 47.8 (16.85) | 48.1 (17.30) |
| | Median | 44.5 | 46.0 | 42.5 | 44.5 | 45.0 |
| | Min, Max | 25, 94 | 26, 86 | 25, 99 | 25, 98 | 25, 99 |
| Week 4 | n | 134 | 82 | 106 | 104 | 426 |
| | Mean (SD) | 38.9 (19.67) | 41.0 (23.73) | 39.0 (18.78) | 37.8 (18.33) | 39.1 (19.95) |
| | Median | 36.0 | 35.0 | 36.5 | 35.0 | 36.0 |
| | Min, Max | 3, 96 | 1, 128 | 8, 107 | 3, 101 | 1, 128 |
| | Change from | | | | | |
| | Baseline | | | | | |
| | n | 134 | 82 | 106 | 104 | 426 |
| | Mean (SD) | 8.6 (15.30) | 8.9 (15.31) | 9.6 (14.87) | 10.2 (12.06) | 9.3 (14.43) |
| | Median | 9.5 | 11.0 | 9,0 | 10.0 | 10.0 |
| | Min, Max | −41, 47 | −48, 37 | −55, 50 | −16, 52 | −55, 52 |
| | Percent Change | | | | | |
| | from Baseline | | | | | |
| | n | 134 | 82 | 106 | 104 | 426 |
| | Mean (SD) | 17.34 (33.197) | 19.34 (31.227) | 17,19 (36.142) | 21.28 (26.625) | 18.65 (32.064) |
| | Median | 19.48 | 22.57 | 20.27 | 16.98 | 19.80 |
| | Min, Max | −141.4, 92.2 | −77.4, 97.4 | −220.0, 84.0 | −57.7, 93.5 | −220.0, 97.4 |
| Week 8 | n | 128 | 78 | 101 | 104 | 411 |
| | Mean (SD) | 29.7 (17.71) | 33.8 (18.78) | 33.5 (19.23) | 30.3 (16.98) | 31.5 (18.15) |
| | Median | 27.0 | 29.5 | 31.0 | 27.0 | 28.0 |
| | Min, Max | 1, 83 | 2, 94 | 4, 102 | 0, 75 | 0, 102 |
| | Change from | | | | | |
| | Baseline | | | | | |
| | n | 128 | 78 | 101 | 104 | 411 |
| | Mean (SD) | 17.6 (16.34) | 16.3 (15.76) | 15.3 (18.70) | 17.5 (13.95) | 16.8 (16.26) |
| | Median | 18.0 | 19.0 | 16.0 | 14.5 | 18.0 |
| | Min, Max | −24, 58 | −44, 57 | −77, 64 | −13, 72 | −77, 72 |
| | Percent Change | | | | | |
| | from Baseline | | | | | |
| | n | 128 | 78 | 101 | 104 | 411 |
| | Mean (SD) | 36.58 (32.444) | 31.51 (36.538) | 28.25 (46.676) | 37.03 (25.956) | 33.69 (35.931) |
| | Median | 41.19 | 36.08 | 36.49 | 36.66 | 37.74 |
| | Min, Max | −52.8, 96.2 | −129.4, 94.6 | −308.0, 90.5 | −34.2, 100.0 | −308.0, 100.0 |
| Week 12 | n | 128 | 76 | 104 | 99 | 407 |
| | Mean (SD) | 21.9 (16.52) | 25.8 (17.16) | 29.4 (22.41) | 27.2 (16.82) | 25.9 (18.55) |
| | Median | 18.5 | 22.0 | 26.5 | 23.0 | 22.0 |
| | Min, Max | 0, 78 | 0, 89 | 0, 119 | 1, 75 | 0, 119 |
| | Change from | | | | | |
| | Baseline | | | | | |
| | n | 128 | 76 | 104 | 99 | 407 |
| | Mean (SD) | 25.9 (17.08) | 24.1 (15.66) | 19.7 (23.05) | 21.1 (15.62) | 22.8 (18.35) |
| | Median | 24.5 | 23.0 | 21.5 | 22.0 | 23.0 |
| | Min, Max | −33, 68 | −11, 72 | −82, 73 | −21, 73 | −82, 73 |
| | Percent Change | | | | | |
| | from Baseline | | | | | |
| | n | 128 | 76 | 104 | 99 | 407 |
| | Mean (SD) | 53.83 (32.145) | 48.09 (27.988) | 38.10 (49.589) | 43.21 (30.062) | 46.15 (36.714) |
| | Median | 60.43 | 52.40 | 48.70 | 47.62 | 52.63 |
| | Min, Max | −73.3, 100.0 | −33.3, 100.0 | −248.5, 100.0 | −53.8, 97.6 | −248.5, 100.0 |

Note:
Changes from baseline lesion counts are calculated as the baseline value minus the post-baseline value.
Note:
Percent changes from baseline lesion count are calculated as the baseline value minus the post-baseline value divided by the baseline value, expressed as a percentage.
Note:
OC = Observed Cases;
LOCF = Last Observation Carried Forward;
BOCF = Baseline Observation Carried Forward.

TABLE 48B
Analysis of Change from Baseline In Non-Inflammatory Lesion
Count at Week 12-by Different Imputations of ITT Population

| Imputation | | FCD105 | Vehicle |
|---|---|---|---|
| Method | Statistics | (N = 142) | (N = 83) |
| OC | LSMean ^{[1]} | 25.53 | 23.02 |
| | Std Err LSMean | 1.66 | 1.90 |
| | 95% CI of LSMean | 22.26-28.81 | 19.26-26.77 |
| | LSMean Difference | 2.52 | |
| | Std Err Difference | 2.14 | |
| | 95% CI of LSMean Difference | −1.71-6.74 | |
| | P-value ^{[2]} | 0.2417 | |
| LOCH | LSMean ^{[1]} | 23.62 | 22.13 |
| | Std Err LSMean | 1.63 | 1.89 |
| | 95% CI of LSMean | 20.42-26.83 | 18.40-25.86 |
| | LSMean Difference | 1.50 | |
| | Std Err Difference | 2.11 | |
| | 95% CI of LSMean Difference | −2.66-5.66 | |
| | P-value ^{[2]} | 0.4789 | |
| BOCF | LSMean ^{[1]} | 23.22 | 21.05 |
| | Std Err LSMean | 1.67 | 1.95 |
| | 95% CI of LSMean | 19.93-26.50 | 17.19-24.90 |
| | LSMean Difference | 2.17 | |
| | Std Err Difference | 2.20 | |
| | 95% CIof LSMean Difference | −2.18-6.52 | |
| | P-value ^{[2]} | 0.3256 | |
| MI | LSMean ^{[1]} | 24.94 | 22.87 |
| | Std Err LSMean | 1.61 | 1.87 |
| | 95% CI of LSMean | 21.78-28.10 | 19.21-26.53 |
| | LSMean Difference | 2.07 | |
| | Std Err Difference | 2.09 | |
| | 95% CI of LSMean Difference | −2.02-6.17 | |
| | P-value ^{[2]} | 0.3210 | |

Note:
Changes from baseline lesion counts are calculated as the baseline value minus the post-baseline value.
^{[1]} Least Squares Means are defined as model-based linear combination (sum) of the estimated effects.
^{[2]} P-value obtains from ANCOVA repeated measures model with treatment and visit as main effect, baseline inflammatory lesion count as a covariate, and analysis center as a blocking factor.
Note:
OC = Observed Cases;
LOCF = Last Observation Carried Forward;
BOCF = Baseline Observation Carried Forward;
MI = Multiple Imputation.

TABLE 48C
Analysis of Ranked Change from Baseline In Non-Inflammatory
Lesion Count at Week 12-Multiple Imputation of ITT Population

| | FCD105 | Vehicle |
|---|---|---|
| Statistics | (N = 142) | (N = 83) |
| LSMean ^{[1]} | 113.88 | 123.64 |
| Std Err LSMean | 6.22 | 7.19 |
| 95% CI of LSMean | 101.69-126.08 | 109.54-137.74 |
| LSMean Difference | −9.76 | |
| Std Err Difference | 8.07 | |
| 95% CI of LSMean Difference | −25.58-6.06 | |
| P-value ^{[2]} | 0.2265 | |

Note:
Changes from baseline lesion counts are calculated as the baseline value minus the post-baseline value.
^{[1]} Least Squares Means are defined as model-based linear combination (sum) of the estimated effects.
^{[2]} P-value obtains from ANCOVA repeated measures model with treatment and visit as main effect, baseline inflammatory lesion count as a covariate, and analysis center as a blocking factor.

TABLE 48D
Analysis of Percent Change from Baseline in Non-Inflammatory
Lesion Count-Multiple Imputation (Week 12, Week 8 and Week 4)-ITT
Population

| | FCD105 | Vehicle |
|---|---|---|
| Statistics | (N = 142) | (N = 83) |
| Week 12 | | |
| LSMean ^{[1]} | 50.95 | 45.91 |
| Std Err LSMean | 3.35 | 3.87 |
| 95% CI of LSMean | 44.37-57.52 | 38.33-53.49 |
| LSMean Difference | 5.04 | |
| Std Err Difference | 4.33 | |
| 95% CI of LSMean Difference | −3.46-13.53 | |
| P-value ^{[2]} | 0.2452 | |
| Week 8 | | |
| LSMean ^{[1]} | 34.27 | 29.89 |
| Std Err LSMean | 3.59 | 4.17 |
| 95% CI of LSMean | 27.24-41.30 | 21.71-38.07 |
| LSMean Difference | 4.38 | |
| Std Err Difference | 4.76 | |
| 95% CI of LSMean Difference | −4.95-13.71 | |
| P-value ^{[2]} | 0.3573 | |
| Week 4 | | |
| LSMean ^{[1]} | 15.25 | 17.26 |
| Std Err LSMean | 3.55 | 4.06 |
| 95% CI of LSMean | 8.28-22.22 | 9.30-25.22 |
| LSMean Difference | −2.01 | |
| Std Err Difference | 4.63 | |
| 95% CI of LSMean Difference | −11.09-7.06 | |
| P-value ^{[2]} | 0.6641 | |

Note:
Percent changes from baseline lesion count are calculated as the baseline value minus the post-baseline value divided by the baseline value, expressed as a percentage.
Note:
OC = Observed Cases;
LOCF = Last Observation Carried Forward;
BOCF = Baseline Observation Carried Forward.
^{[1]} Least Squares Means are defined as model-based linear combination (sum) of the estimated effects.
^{[2]} P-value obtains from ANCOVA repeated measures model with treatment and visit as main effect, baseline non-inflammatory lesion count as a covariate, and analysis center as a blocking factor.

TABLE 48E
Analysis of Change from Baseline in Non-Inflammatory Lesion
Count-Secondary-Multiple Imputation-ITT Population

| | FCD105 | Minocycline 3% | Adapalene 0.3% |
|---|---|---|---|
| Statistics | (N = 142) | (N = 110) | (N = 112) |
| Week 12 | | | |
| LSMean ^{[1]} | 25.38 | 19.08 | 20.70 |
| Std Err LSMean | 1.61 | 1.72 | 1.76 |
| 95% CI of LSMean | 22.23-28.54 | 15.71-22.46 | 17.24-24.15 |
| LSMean Difference | | −6.30 | −4.69 |
| Std Err Difference | | 2.14 | 2.15 |
| 95% CI of LSMean | | −10.50-−2.10 | −8.90-−0.47 |
| Difference | | | |
| P-value ^{[2]} | | 0.0033 | 0.0292 |
| Week 8 | | | |
| LSMean ^{[1]} | 17.76 | 14.87 | 17.46 |
| Std Err LSMean | 1.47 | 1.56 | 1.59 |
| 95% CI of LSMean | 14.88-20.65 | 11.82-17.92 | 14.35-20.58 |
| LSMean Difference | | −2.89 | −0.30 |
| Std Err Difference | | 1.91 | 1.91 |
| 95% CI of LSMean | | −6.63-0.84 | −4.04-3.44 |
| Difference | | | |
| P-value ^{[2]} | | 0.1291 | 0.8754 |
| Week 4 | | | |
| LSMean ^{[1]} | 8.72 | 9.33 | 10.04 |
| Std Err LSMean | 1.39 | 1.45 | 1.48 |
| 95% CI of LSMean | 6.00-11.43 | 6.49-12.17 | 7.14-12.93 |
| LSMean Difference | | 0.62 | 1.32 |
| Std Err Difference | | 1.73 | 1.74 |
| 95% CI of LSMean | | −2.78-4.01 | −2.09-4.73 |
| Difference | | | |
| P-value ^{[2]} | | 0.7218 | 0.4486 |

Note:
Changes from baseline lesion counts are calculated as the baseline value minus the post-baseline value.
^{[1]} Least Squares Means are defined as model-based linear combination (sum) of the estimated effects.
^{[2]} P-value obtains from ANCOVA repeated measures model with treatment and visit as main effect, baseline non-inflammatory lesion count as a covariate, and analysis center as a blocking factor.

TABLE 49A
Descriptive Summary of Non-Inflammatory Lesion Count-Per-
Protocol Population

| | | FCD105 | Vehicle | Minocycline 3% | Adapalene 0.3% | Total |
|---|---|---|---|---|---|---|
| Visit | Statistics | (N = 142) | (N = 82) | (N = 110) | (N = 111) | (N = 445) |
| Baseline | n | 142 | 82 | 110 | 111 | 445 |
| | Mean (SD) | 47.2 (16.74) | 49.9 (16.59) | 48.4 (19.08) | 47.7 (16.91) | 48.1 (17.33) |
| | Median | 44.5 | 46.0 | 42.5 | 44.0 | 45.0 |
| | Min, Max | 25, 94 | 26, 86 | 25, 99 | 25, 98 | 25, 99 |
| Week 4 | n | 134 | 82 | 106 | 103 | 425 |
| | Mean (SD) | 38.9 (19.67) | 41.0 (23.73) | 39.0 (18.78) | 37.7 (18.41) | 39.1 (19.97) |
| | Median | 36.0 | 35.0 | 36.5 | 35.0 | 36.0 |
| | Min, Max | 3, 96 | 1, 128 | 8, 107 | 3, 101 | 1, 128 |
| | Change from | | | | | |
| | Baseline | | | | | |
| | n | 134 | 82 | 106 | 103 | 425 |
| | Mean (SD) | 8.6 (15.30) | 8.9 (15.31) | 9.6 (14.87) | 10.1 (12.11) | 9.3 (14.45) |
| | Median | 9.5 | 11.0 | 9.0 | 10.0 | 10.0 |
| | Min, Max | −41, 47 | −48, 37 | −55, 50 | −16, 52 | −55, 52 |
| | Percent Change | | | | | |
| | from Baseline | | | | | |
| | n | 134 | 82 | 106 | 103 | 425 |
| | Mean (SD) | 17.34 (33.197) | 19.34 (31.227) | 17.19 (36.142) | 21.25 (26.754) | 18.64 (32.100) |
| | Median | 19.48 | 22.57 | 20.27 | 16.22 | 19.61 |
| | Min, Max | −141.4, 92.2 | −77.4, 97.4 | −220.0, 84.0 | −57.7, 93.5 | −220.0, 97.4 |
| Week 8 | n | 128 | 78 | 101 | 103 | 410 |
| | Mean (SD) | 29.7 (17.71) | 33.8 (18.78) | 33.5 (19.23) | 30.3 (17.06) | 31.6 (18.17) |
| | Median | 27.0 | 29.5 | 31.0 | 27.0 | 28.5 |
| | Min, Max | 1, 83 | 2, 94 | 4, 102 | 0, 75 | 0, 102 |
| | Change from | | | | | |
| | Baseline | | | | | |
| | n | 128 | 78 | 101 | 103 | 410 |
| | Mean (SD) | 17.6 (16.34) | 16.3 (15.76) | 15.3 (18.70) | 17.4 (13.97) | 16.7 (16.27) |
| | Median | 18.0 | 19.0 | 16.0 | 14.0 | 17.5 |
| | Min, Max | −24, 58 | −44, 57 | −77, 64 | −13,72 | −77,72 |
| | Percent Change | | | | | |
| | from Baseline | | | | | |
| | n | 128 | 78 | 101 | 103 | 410 |
| | Mean (SD) | 36.58 (32.444) | 31.51 (36.538) | 28.25 (46.676) | 36.90 (26.046) | 33.65 (35.965) |
| | Median | 41.19 | 36.08 | 36.49 | 36.59 | 37.62 |
| | Min, Max | −52.8, 96.2 | −129.4, 94.6 | −308.0, 90.5 | −34.2, 100.0 | −308.0, 100.0 |
| Week 12 | n | 128 | 76 | 104 | 98 | 406 |
| | Mean (SD) | 21.9 (16.52) | 25.8 (17.16) | 29.4 (22.41) | 27.2 (16.91) | 25.9 (18.57) |
| | Median | 18.5 | 22.0 | 26.5 | 22.5 | 22.0 |
| | Min, Max | 0, 78 | 0, 89 | 0, 119 | 1, 75 | 0, 119 |
| | Change from | | | | | |
| | Baseline | | | | | |
| | n | 128 | 76 | 104 | 98 | 406 |
| | Mean (SD) | 25.9 (17.08) | 24.1 (15.66) | 19.7 (23.05) | 21.0 (15.68) | 22.8 (18.37) |
| | Median | 24.5 | 23.0 | 21.5 | 21.5 | 23.0 |
| | Min, Max | −33, 68 | −11, 72 | −82, 73 | −21, 73 | −82,73 |
| | Percent Change | | | | | |
| | from Baseline | | | | | |
| | n | 128 | 76 | 104 | 98 | 406 |
| | Mean (SD) | 53.83 (32.145) | 48.09 (27.988) | 38.10 (49.589) | 43.12 (30.201) | 46.14 (36.758) |
| | Median | 60.43 | 52.40 | 48.70 | 47.10 | 53.10 |
| | Min, Max | −73.3, 100.0 | −33.3, 100.0 | −248.5, 100.0 | −53.8, 97.6 | −248.5, 100.0 |

Note:
Changes from baseline lesion counts are calculated as the baseline value minus the post-baseline value.
Note:
Percent changes from baseline lesion count are calculated as the baseline value minus the post-baseline value divided by the baseline value, expressed as a percentage.
Note:
OC = Observed Cases;
LOCF = Last Observation Carried Forward;
BOCF = Baseline Observation Carried Forward.

TABLE 49B
Analysis of Change from Baseline In Non-Inflammatory Lesion
Count at Week 12-Per-Protocol Population

| | FCD105 | Vehicle |
|---|---|---|
| Statistics | (N = 142) | (N = 82) |
| LSMean ^{[1]} | 25.53 | 23.02 |
| Std Err LSMean | 1.66 | 1.90 |
| 95% CI of LSMean | 22.26-28.81 | 19.26-26.77 |
| LSMean Difference | 2.52 | |
| Std Err Difference | 2.14 | |
| 95% CI of LSMean Difference | −1.71-6.74 | |
| P-value ^{[2]} | 0.2417 | |

Note:
Changes from baseline lesion counts are calculated as the baseline value minus the post-baseline value.
Note:
Percent changes from baseline lesion count are calculated as the baseline value minus the post-baseline value divided by the baseline value, expressed as a percentage.

TABLE 49C
Analysis of Percent Change from Baseline in Non-Inflammatory
Lesion Count (Week 12, Week 8 and Week 4)-Per-Protocol Population

| | FCD105 | Vehicle |
|---|---|---|
| Statistics | (N = 142) | (N = 82) |
| Week 12 | | |
| LSMean ^{[1]} | 51.87 | 46.07 |
| Std Err LSMean | 3.39 | 3.87 |
| 95% CI of LSMean | 45.19-58.54 | 38.44-53.69 |
| LSMean Difference | 5.80 | |
| Std Err Difference | 4.33 | |
| 95% CI of LSMean Difference | −2.74-14.35 | |
| P-value ^{[2]} | 0.1820 | |
| Week 8 | | |
| LSMean ^{[1]} | 34.88 | 30.30 |
| Std Err LSMean | 3.61 | 4.16 |
| 95% CI of LSMean | 27.77-41.99 | 22.09-38.50 |
| LSMean Difference | 4.58 | |
| Std Err Difference | 4.74 | |
| 95% CI of LSMean Difference | −4.77-13.94 | |
| P-value ^{[2]} | 0.3348 | |
| Week 4 | | |
| LSMean ^{[1]} | 15.72 | 17.35 |
| Std Err LSMean | 3.52 | 4.04 |
| 95% CI of LSMean | 8.78-22.67 | 9.38-25.31 |
| LSMean Difference | −1.62 | |
| Std Err Difference | 4.57 | |
| 95% CI of LSMean Difference | −10.64-7.39 | |
| P-value ^{[2]} | 0.7229 | |

Note:
Percent changes from baseline lesion count are calculated as the baseline value minus the post-baseline value divided by the baseline value, expressed as a percentage.
Note:
OC = Observed Cases;
LOCF = Last Observation Carried Forward;
BOCF = Baseline Observation Carried Forward.
^{[1]} Least Squares Means are defined as model-based linear combination (sum) of the estimated effects.
^{[2]} P-value obtains from ANCOVA repeated measures model with treatment and visit as main effect, baseline non-inflammatory lesion count as a covariate, and analysis center as a blocking factor.

TABLE 49D
Analysis of Change from Baseline in Non-Inflammatory Lesion
Count-Secondary-Multiple Imputation-Per-Protocol Population

| | FCD105 | Minocycline 3% | Adapalene 0.3% |
|---|---|---|---|
| Statistics | (N = 142) | (N = 110) | (N = 111) |
| Week 12 | | | |
| LSMean ^{[1]} | 26.04 | 19.44 | 21.06 |
| Std Err LSMean | 1.66 | 1.75 | 1.80 |
| 95% CI of LSMean | 22.78-29.30 | 16.00-22.89 | 17.51-24.61 |
| LSMean Difference | | −6.60 | −4.98 |
| Std Err Difference | | 2.19 | 2.22 |
| 95% CI of LSMean | | −10.91-−2.29 | −9.34-−0.62 |
| Difference | | | |
| P-value ^{[2]} | | 0.0028 | 0.0252 |
| Week 8 | | | |
| LSMean ^{[1]} | 18.18 | 15.10 | 17.59 |
| Std Err LSMean | 1.50 | 1.58 | 1.60 |
| 95% CI of LSMean | 15.23-21.14 | 11.99-18.21 | 14.43-20.74 |
| LSMean Difference | | −3.08 | −0.60 |
| Std Err Difference | | 1.93 | 1.93 |
| 95% CI of LSMean | | −6.88-0.72 | −4.40-3.21 |
| Difference | | | |
| P-value ^{[2]} | | 0.1117 | 0.7572 |
| Week 4 | | | |
| LSMean ^{[1]} | 9.07 | 9.55 | 10.15 |
| Std Err LSMean | 1.39 | 1.46 | 1.49 |
| 95% CI of LSMean | 6.33-11.80 | 6.69-12.41 | 7.22-13.09 |
| LSMean Difference | | 0.48 | 1.09 |
| Std Err Difference | | 1.73 | 1.74 |
| 95% CI of LSMean | | −2.92-3.89 | −2.34-4.51 |
| Difference | | | |
| P-value ^{[2]} | | 0.7805 | 0.5321 |

Note:
Changes from baseline lesion counts are calculated as the baseline value minus the post-baseline value.
^{[1]} Least Squares Means are defined as model-based linear combination (sum) of the estimated effects.
^{[2]} P-value obtains from ANCOVA repeated measures model with treatment and visit as main effect, baseline non-inflammatory lesion count as a covariate, and analysis center as a blocking factor.

### Patients' Satisfaction

The degree of satisfaction of the patients from the treatment with the investigated products was evaluated based on patient's questionnaire results after 12 weeks of treatment (Table 50). In addition, two questions were also evaluated at baseline and after 12 weeks of treatment 1. “How would you rate your facial acne?” and 2. “I am bothered by the appearance of my facial skin?”

Although statistical analysis was not conducted, overall patients' satisfaction was high in the FCD′ 105 treatment group as compared to the other treatment groups and numerically superior for most of the questions evaluated.

Most patients had rated their acne as moderate at baseline in all treatment groups. The most pronounced numerical reduction after 12 weeks for this assessment was in patients in the FCD′105 treatment group −46% as compared to −31.3% in vehicle treatment group, −34.3% in minocycline group and 22.2% in the adapalene group. Amongst patients who rated their acne as severe, a reduction from double to single figures was seen across all the groups.

66.2% of patients in the FCD′105 treatment group, stated that they were satisfied or very satisfied with this product treating their acne, as compared to 53% in vehicle group, 51.8% in the minocycline treatment group and 39.3% in the retinoid group. 39.4% of patients in the FCD′105 treatment group stated that they would very likely recommend the product to a friend as compared to 37.3% in the minocycline group 36.1% in the vehicle group and 28.6% in the adapalene group. In the overall satisfaction FCD105 treatment group also ranked the highest with 26.8% of the patients who responded that they were very satisfied followed by minocycline foam, retinoid foam and lastly vehicle foam. When comparing the very satisfied and satisfied groups together the retinoid foam came last. Nevertheless, when adapalene is combined with minocycline in the combination product, FCD105 patients' satisfaction was not merely higher but patients indicated that the combination was the preferred product.

As for overall ease of use of the product, 85.5% of the patients in the FCD105 group said that the product was very easy or easy to use as compared to 83.6% in the minocycline group 81.3% in the adapalene group and 80.7% in the vehicle group.

TABLE 50
Descriptive Summary of Subject Satisfaction Questionnaire - ITT Population

| | FCD105 | Vehicle | Minocycline 3% | Adapalene 0.3% | Total |
|---|---|---|---|---|---|
| | (N = 142) | (N = 83) | (N = 110) | (N = 112) | (N = 447) |
| Baseline | | | | | |
| 1. How would you rate your facial | | | | | |
| acne? | | | | | |
| Clear | 0 | 1 (1.2%) | 0 | 0 | 1 (0.2%) |
| Minimal | 3 (2.1%) | 3 (3.7%) | 1 (0.9%) | 6 (5.4%) | 13 (2.9%) |
| Mild | 44 (31.0%) | 24 (29.3%) | 32 (29.1%) | 29 (25.9%) | 129 (28.9%) |
| Moderate | 83 (58.5%) | 44 (53.7%) | 58 (52.7%) | 52 (46.4%) | 237 (53.1%) |
| Severe | 12 (8.5%) | 10 (12.2%) | 19 (17.3%) | 25 (22.3%) | 66 (14.8%) |
| 2. I am bothered by the appearance | | | | | |
| of my facial skin | | | | | |
| Not at all Bothered | 10 (7.0%) | 9 (11.0%) | 5 (4.5%) | 8 (7.1%) | 32 (7.2%) |
| Somewhat Bothered | 42 (29.6%) | 25 (30.5%) | 43 (39.1%) | 27 (24.1%) | 137 (30.7%) |
| Bothered | 57 (40.1%) | 18 (22.0%) | 33 (30.0%) | 33 (29.5%) | 141 (31.6%) |
| Very Bothered | 33 (23.2%) | 30 (36.6%) | 29 (26.4%) | 44 (39.3%) | 136 (30.5%) |
| Week 12 | | | | | |
| 1. How would you rate your facial | | | | | |
| acne? | | | | | |
| Clear | 11 (8.6%) | 3 (3.9%) | 5 (4.9%) | 4 (4.0%) | 23 (5.7%) |
| Minima | 41 (32.0%) | 24 (31.6%) | 37 (35.9%) | 26 (26.3%) | 128 (31.5%) |
| Mild | 58 (45.3%) | 31 (40.8%) | 41 (39.8%) | 42 (42.4%) | 172 (42.4%) |
| Moderate | 16 (12.5%) | 17 (22.4%) | 19 (18.4%) | 24 (24.2%) | 76 (18.7%) |
| Severe | 2 (1.6%) | 1 (1.3%) | 1 (1.0%) | 3 (3.0%) | 7 (1.7%) |
| 2. I am bothered by the appearance | | | | | |
| of my facial skin | | | | | |
| Not at all Bothered | 33 (25.8%) | 15 (19.7%) | 24 (23.3%) | 23 (23.2%) | 95 (23.4%) |
| Somewhat Bothered | 67 (52.3%) | 41 (53.9%) | 61 (59.2%) | 50 (50.5%) | 219 (53.9%) |
| Bothered | 18 (14.1%) | 14 (18.4%) | 13 (12.6%) | 15 (15.2%) | 60 (14.8%) |
| Very Bothered | 10 (7.8%) | 6 (7.9%) | 5 (4.9%) | 11 (11.1%) | 32 (7.9%) |
| 3. How satisfied are you with this | | | | | |
| product in treating your acne? | | | | | |
| Very Satisfied | 30 (23.4%) | 16 (21.1%) | 27 (26.2%) | 17 (17.2%) | 90 (22.2%) |
| Satisfied | 64 (50.0%) | 28 (36.8%) | 30 (29.1%) | 27 (27.3%) | 149 (36.7%) |
| Somewhat Satisfied | 28 (21.9%) | 24 (31.6%) | 38 (36.9%) | 42 (42.4%) | 132 (32.5%) |
| Dissatisfied | 4 (3.1%) | 8 (10.5%) | 7 (6.8%) | 11 (11.1%) | 30 (7.4%) |
| Very Dissatisfied | 2 (1.6%) | 0 | 1 (1.0%) | 2 (2.0%) | 5 (1.2%) |
| 4. How satisfied are you with the | | | | | |
| odor of this product after treatment? | | | | | |
| Very Satisfied | 27 (21.1%) | 15 (19.7%) | 35 (34.0%) | 19 (19.2%) | 96 (23.6%) |
| Satisfied | 60 (46.9%) | 39 (51.3%) | 33 (32.0%) | 51 (51.5%) | 183 (45.1%) |
| Somewhat Satisfied | 29 (22.7%) | 16 (21.1%) | 21 (20.4%) | 22 (22.2%) | 88 (21.7%) |
| Dissatisfied | 11 (8.6%) | 5 (6.6%) | 13 (12.6%) | 6 (6.1%) | 35 (8.6%) |
| Very Dissatisfied | 1 (0.8%) | 1 (1.3%) | 1 (1.0%) | 1 (1.0%) | 4 (1.0%) |
| 5. How satisfied are you with the | | | | | |
| color of this product after treatment? | | | | | |
| Very Satisfied | 17 (13.3%) | 6 (7.9%) | 21 (20.4%) | 8 (8.1%) | 52 (12.8%) |
| Satisfied | 45 (35.2%) | 18 (23.7%) | 36 (35.0%) | 35 (35.4%) | 134 (33.0%) |
| Somewhat Satisfied | 27 (21.1%) | 19 (25.0%) | 23 (22.3%) | 22 (22.2%) | 91 (22.4%) |
| Dissatisfied | 28 (21.9%) | 27 (35.5%) | 15 (14.6%) | 24 (24.2%) | 94 (23.2%) |
| Very Dissatisfied | 11 (8.6%) | 6 (7.9%) | 8 (7.8%) | 10 (10.1%) | 35 (8.6%) |
| 6. How satisfied are you with how | | | | | |
| easy this product is to put on your | | | | | |
| skin? | | | | | |
| Very Satisfied | 63 (49.2%) | 32 (42.1%) | 51 (49.5%) | 37 (37.4%) | 183 (45.1%) |
| Satisfied | 49 (38.3%) | 30 (39.5%) | 35 (34.0%) | 44 (44.4%) | 158 (38.9%) |
| Somewhat Satisfied | 13 (10.2%) | 12 (15.8%) | 13 (12.6%) | 15 (15.2%) | 53 (13.1%) |
| Dissatisfied | 3 (2.3%) | 1 (1.3%) | 4 (3.9%) | 2 (2.0%) | 10 (2.5%) |
| Very Dissatisfied | 0 | 1 (1.3%) | 0 | 1 (1.0%) | 2 (0.5%) |
| 7. How moisturizing did this | | | | | |
| product feel on your skin? | | | | | |
| Very Moisturizing | 33 (25.8%) | 32 (42.1%) | 32 (31.1%) | 26 (26.3%) | 123 (30.3%) |
| Moisturizing | 43 (33.6%) | 13 (17.1%) | 28 (27.2%) | 29 (29.3%) | 113 (27.8%) |
| Neutral | 40 (31.3%) | 28 (36.8%) | 38 (36.9%) | 26 (26.3%) | 132 (32.5%) |
| Drying | 11 (8.6%) | 3 (3.9%) | 4 (3.9%) | 15 (15.2%) | 33 (8.1%) |
| Very Drying | 1 (0.8%) | 0 | 1 (1.0%) | 3 (3.0%) | 5 (1.2%) |
| 8. How satisfied are you with the | | | | | |
| feel of this product on your skin | | | | | |
| after treatment? | | | | | |
| Very Satisfied | 26 (20.3%) | 10 (13.2%) | 16 (15.5%) | 12 (12.1%) | 64 (15.8%) |
| Satisfied | 54 (42.2%) | 29 (38.2%) | 39 (37.9%) | 44 (44.4%) | 166 (40.9%) |
| Somewhat Satisfied | 29 (22.7%) | 30 (39.5%) | 29 (28.2%) | 23 (23.2%) | 111 (27.3%) |
| Dissatisfied | 14 (10.9%) | 5 (6.6%) | 15 (14.6%) | 17 (17.2%) | 51 (12.6%) |
| Very Dissatisfied | 5 (3.9%) | 2 (2.6%) | 4 (3.9%) | 3 (3.0%) | 14 (3.4%) |
| 9. Was the product easy to | | | | | |
| dispense from the canister? | | | | | |
| Very Easy | 93 (72.7%) | 53 (69.7%) | 72 (69.9%) | 68 (68.7%) | 286 (70.4%) |
| Easy | 31 (24.2%) | 20 (26.3%) | 28 (27.2%) | 28 (28.3%) | 107 (26.4%) |
| Some Difficulty | 4 (3.1%) | 3 (3.9%) | 3 (2.9%) | 3 (3.0%) | 13 (3.2%) |
| Very Hard to Dispense | 0 | 0 | 0 | 0 | 0 |
| 10. Was the amount of product | | | | | |
| dispensed at each time of use from | | | | | |
| the canister, in line with expectations? | | | | | |
| Much more than expected | 12 (9.4%) | 8 (10.5%) | 7 (6.8%) | 8 (8.1%) | 35 (8.6%) |
| More than expected | 33 (25.8%) | 18 (23.7%) | 33 (32.0%) | 17 (17.2%) | 101 (24.9%) |
| As expected | 78 (60.9%) | 47 (61.8%) | 62 (60.2%) | 69 (69.7%) | 256 (63.1%) |
| Less than expected | 5 (3.9%) | 2 (2.6%) | 1 (1.0%) | 5 (5.1%) | 13 (3.2%) |
| Much less than expected | 0 | 1 (1.3%) | 0 | 0 | 1 (0.2%) |
| 11. Overall, how easy was it to use | | | | | |
| the product? | | | | | |
| Very Easy | 82 (64.1%) | 46 (60.5%) | 67 (65.0%) | 60 (60.6%) | 255 (62.8%) |
| Easy | 39 (30.5%) | 21 (27.6%) | 25 (24.3%) | 31 (31.3%) | 116 (28.6%) |
| Neutral | 6 (4.7%) | 7 (9.2%) | 9 (8.7%) | 7 (7.1%) | 29 (7.1%) |
| Some Difficulty | 1 (0.8%) | 2 (2.6%) | 2 (1.9%) | 1 (1.0%) | 6 (1.5%) |
| Very Hard | 0 | 0 | 0 | 0 | 0 |
| 12. Overall, how satisfied are you | | | | | |
| with this product? | | | | | |
| Very Satisfied | 38 (29.7%) | 15 (19.7%) | 27 (26.2%) | 22 (22.2%) | 102 (25.1%) |
| Satisfied | 51 (39.8%) | 33 (43.4%) | 44 (42.7%) | 35 (35.4%) | 163 (40.1%) |
| Somewhat Satisfied | 31 (24.2%) | 22 (28.9%) | 25 (24.3%) | 32 (32.3%) | 110 (27.1%) |
| Dissatisfied | 7 (5.5%) | 6 (7.9%) | 6 (5.8%) | 8 (8.1%) | 27 (6.7%) |
| Very Dissatisfied | 1 (0.8%) | 0 | 1 (1.0%) | 2 (2.0%) | 4 (1.0%) |
| 13. Overall, how likely are you to | | | | | |
| recommend this product to a friend? | | | | | |
| Very likely | 56 (43.8%) | 30 (39.5%) | 41 (39.8%) | 32 (32.3%) | 159 (39.2%) |
| Somewhat likely | 48 (37.5%) | 20 (26.3%) | 32 (31.1%) | 34 (34.3%) | 134 (33.0%) |
| Neutral | 14 (10.9%) | 17 (22.4%) | 21 (20.4%) | 21 (21.2%) | 73 (18.0%) |
| Somewhat unlikely | 9 (7.0%) | 8 (10.5%) | 6 (5.8%) | 7 (7.1%) | 30 (7.4%) |
| Very unlikely | 1 (0.8%) | 1 (1.3%) | 3 (2.9%) | 5 (5.1%) | 10 (2.5%) |

Overall, treatment emergent adverse events in the safety population were few in type and frequency (e.g., group 3.5% in FCD105 treatment compared to 8.9% in the 0.3% adapalene treatment group, 1.8% in the 3% minocycline treatment group and none in the vehicle treatment group) and all of them were resolved prior to study completion. The most commonly reported treatment emergent adverse event in the trial was upper respiratory tract infection (4.9% in the vehicle treatment group) with dry skin being the most commonly reported cutaneous and subcutaneous tissue adverse event (3.6% in the 0.3% adapalene treatment group compared to 1.4% in the FCD105 treatment group). All groups received with or without drug an oil-based foam that potentially can help dry skin. As no dry skin events were reported for the vehicle and most dry skin events reported occurred with the adapalene only group, the dry skin events appear to be associated with application of adapalene. No viral upper respiratory tract infections were reported for the combination product or minocycline alone. Of the adverse events the majority were assessed as mild in severity (e.g., for FCD105 treatment group 8.5% compared to 12.5% in the adapalene treatment group). There were no treatment-emergent serious adverse events in any of the treatment groups. (see Table 51A-B).

Two (1.8% of) subjects receiving 0.3% adapalene only (corresponding to 0.4% of total subjects) experienced what was considered a severe (treatment related) TEAE which led to study drug discontinuation. No severe TEAE's were observed with the combination of adapalene and minocycline. No acne as resultant TEAE was experienced in subjects receiving 3% minocycline or the vehicle. Surprisingly only 0.7% subjects receiving FCD105 treatment experienced mild acne as a resultant TEAE. The severity and/or frequency of most different reported skin and subcutaneous tissue TEAEs experienced in subjects receiving FCD105 treatment was less than that experienced in subjects receiving 0.3% adapalene (see Table 51C). Similar results pertaining to frequency for treatment-related adverse events for skin and subcutaneous tissue disorders and eye disorders were in observed (see Table 51D). The most common treatment-related TEAEs were dry skin, rash, acne, and eye irritation, all of which occurred more frequently in the adapalene 0.3% arm than in the FCD105 or minocycline 3% arm. Without being bound by theory, combination with minocycline may have the ability to mitigate the severity and frequency of adapalene resultant skin subcutaneous tissue disorders TEAEs and this mitigation may have been assisted by the oil-based vehicle.

Subject discontinuations due to a treatment emergent adverse events were low due to dermal TEAE or dermal related TEAE—one patient (0.7%) withdrawing in FCD105 treatment group due to mild acne that was considered unrelated to study drug, compared to three patients (2.7%) in the 0.3% adapalene treatment group withdrawing due to severe acne and mild rash (see Tables 51H and 511). The same three patients (2.7%) in the 0.3% adapalene treatment group withdrawing due to acne and rash were also classified as being subjects withdrawn from study due to treatment-related TEAE (Table 51J).

Safety analysis evaluated physical examination at 12 weeks (Table 52) and vital signs (Table 53) at baseline 4, 8, 12 weeks in addition to AE's and was found to be normal and was comparable to vehicle in all active agent treatment arms.

FCD105 was comparably tolerated to vehicle in all local skin tolerability assessments with over 93% of severity scores being assessed as “none” or “mild” for burning/stinging, itching, dryness, scaling, erythema and hyperpigmentation at week 12. In general, subjects receiving 0.3% adapalene experienced lower overall tolerability (LSTA) scores compared to the other treatment groups (Table 54).

TABLE 51A
Overall Summary of Adverse Events - Safety Population

| | FCD105 | Vehicle | Minocycline 3% | Adapalene 0.3% | Total |
|---|---|---|---|---|---|
| | (N = 142) | (N = 82) | (N = 110) | (N = 112) | (N = 446) |
| AEs reported | | | | | |
| Subjects with at | 21 (14.8%) | 10 (12.2%) | 15 (13.6%) | 20 (17.9%) | 66 (14.8%) |
| least one AE | | | | | |
| Maximum Severity | | | | | |
| Mild | 12 (8.5%) | 7 (8.5%) | 13 (11.8%) | 14 (12.5%) | 46 (10.3%) |
| Moderate | 9 (6.3%) | 3 (3.7%) | 2 (1.8%) | 4 (3.6%) | 18 (4.0%) |
| Severe | 0 | 0 | 0 | 2 (1.8%) | 2 (0.4%) |
| Treatment-Related | 5 (3.5%) | 0 | (1.8%) | 10 (8.9%) | 17 (3.8%) |
| AE | | | | | |
| Serious AE | 0 | 0 | 0 | 0 | 0 |
| Subjects with any | 1 (0.7%) | 0 | 0 | 3 (2.7%) | 4 (0.9%) |
| TEAE leading to | | | | | |
| discontinuation of | | | | | |
| study drug | | | | | |
| Death | 0 | 0 | 0 | 0 | 0 |

Note:
AEs are coded using MedDRA v22.0.
Note:
AE = Adverse Event; MedDRA = Medical Dictionary for Regulatory Activities.

TABLE 51B
Treatment-Emergent Adverse Events by System Organ Class and Preferred Term - Safety Population

| System Organ Class/ | FCD105 | Vehicle | Minocycline 3% | Adapalene 0.3% | Total |
|---|---|---|---|---|---|
| Preferred Term | (N = 142) | (N = 82) | (N = 110) | (N = 112) | (N = 446) |
| Subjects reporting at least one TEAE | 21 (14.8%) | 10 (12.2%) | 15 (13.6%) | 20 (17.9%) | 66 (14.8%) |
| Infections and infestations | 7 (4.9%) | 7 (8.5%) | 9 (8.2%) | 8 (7.1%) | 31 (7.0%) |
| Upper respiratory tract infection | 2 (1.4%) | 4 (4.9%) | 3 (2.7%) | 1 (0.9%) | 10 (2.2%) |
| Nasopharyngitis | 2 (1.4%) | 0 | 3 (2.7%) | 0 | 5 (1.1%) |
| Sinusitis | 1 (0.7%) | 0 | 2 (1.8%) | 1 (0.9%) | 4 (0.9%) |
| Viral upper respiratory tract infection | 0 | 2 (2.4%) | 0 | 1 (0.9%) | 3 (0.7%) |
| Influenza | 1 (0.7%) | 0 | 0 | 1 (0.9%) | 2 (0.4%) |
| Pharyngitis streptococcal | 0 | 1 (1.2%) | 1 (0.9%) | 0 | 2 (0.4%) |
| Acute sinusitis | 0 | 0 | 0 | 1 (0.9%) | 1 (0.2%) |
| Hordeolum | 0 | 0 | 0 | 1 (0.9%) | 1 (0.2%) |
| Infectious mononucleosis | 0 | 1 (1.2%) | 0 | 0 | 1 (0.2%) |
| Otitis media acute | 1 (0.7%) | 0 | 0 | 0 | 1 (0.2%) |
| Pharyngitis | 0 | 0 | 0 | 1 (0.9%) | 1 (0.2%) |
| Staphylococcal infection | 0 | 0 | 0 | 1 (0.9%) | 1 (0.2%) |
| Skin and subcutaneous tissue disorders | 6 (4.2%) | 0 | 3 (2.7%) | 10 (8.9%) | 19 (4.3%) |
| Dry skin | 2 (1.4%) | 0 | 1 (0.9%) | 4 (3.6%) | 7 (1.6%) |
| Rash | 2 (1.4%) | 0 | 1 (0.9%) | 2 (1.8%) | 5 (1.1%) |
| Acne | 1 (0.7%) | 0 | 0 | 2 (1.8%) | 3 (0.7%) |
| Dermatitis contact | 1 (0.7%) | 0 | 0 | 0 | 1 (0.2%) |
| Eczema | 0 | 0 | 0 | 1 (0.9%) | 1 (0.2%) |
| Nail discolouration | 0 | 0 | 1 (0.9%) | 0 | 1 (0.2%) |
| Pain of skin | 1 (0.7%) | 0 | 0 | 0 | 1 (0.2%) |
| Skin discolouration | 0 | 0 | 0 | 1 (0.9%) | 1 (0.2%) |
| Skin irritation | 0 | 0 | 0 | 1 (0.9%) | 1 (0.2%) |
| Nervous system disorders | 4 (2.8%) | 3 (3.7%) | 2 (1.8%) | 2 (1.8%) | 11 (2.5%) |
| Headache | 1 (0.7%) | 3 (3.7%) | 2 (1.8%) | 2 (1.8%) | 8 (1.8%) |
| Burning sensation | 1 (0.7%) | 0 | 0 | 0 | 1 (0.2%) |
| Hyperaesthesia | 1 (0.7%) | 0 | 0 | 0 | 1 (0.2%) |
| Sciatica | 1 (0.7%) | 0 | 0 | 0 | 1 (0.2%) |
| Gastrointestinal disorders | 5 (3.5%) | 1 (1.2%) | 1 (0.9%) | 1 (0.9%) | 8 (1.8%) |
| Diarrhoea | 1 (0.7%) | 1 (1.2%) | 0 | 1 (0.9%) | 3 (0.7%) |
| Vomiting | 2 (1.4%) | 0 | 1 (0.9%) | 0 | 3 (0.7%) |
| Nausea | 1 (0.7%) | 0 | 1 (0.9%) | 0 | 2 (0.4%) |
| Abdominal pain | 1 (0.7%) | 0 | 0 | 0 | 1 (0.2%) |
| Constipation | 1 (0.7%) | 0 | 0 | 0 | 1 (0.2%) |
| Toothache | 1 (0.7%) | 0 | 0 | 0 | 1 (0.2%) |
| Respiratory, thoracic and mediastinal | 0 | 0 | 3 (2.7%) | 1 (0.9%) | 4 (0.9%) |
| disorders | | | | | |
| Cough | 0 | 0 | 0 | 1 (0.9%) | 1 (0.2%) |
| Oropharyngeal pain | 0 | 0 | 1 (0.9%) | 0 | 1 (0.2%) |
| Paranasal sinus discomfort | 0 | 0 | 1 (0.9%) | 0 | 1 (0.2%) |
| Rhinorrhoea | 0 | 0 | 1 (0.9%) | 0 | 1 (0.2%) |
| Eye disorders | 0 | 0 | 0 | 3 (2.7%) | 3 (0.7%) |
| Eye irritation | 0 | 0 | 0 | 2 (1.8%) | 2 (0.4%) |
| Erythema of eyelid | 0 | 0 | 0 | 1 (0.9%) | 1 (0.2%) |
| General disorders and administration site | 1 (0.7%) | 0 | 1 (0.9%) | 1 (0.9%) | 3 (0.7%) |
| conditions | | | | | |
| Influenza like illness | 1 (0.7%) | 0 | 0 | 1 (0.9%) | 2 (0.4%) |
| Pain | 0 | 0 | 1 (0.9%) | 0 | 1 (0.2%) |
| Injury, poisoning and procedural | 1 (0.7%) | 0 | 0 | 2 (1.8%) | 3 (0.7%) |
| complications | | | | | |
| Burns first degree | 0 | 0 | 0 | 1 (0.9%) | 1 (0.2%) |
| Facial bones fracture | 0 | 0 | 0 | 1 (0.9%) | 1 (0.2% |
| Ligament rupture | 1 (0.7%) | 0 | 0 | 0 | 1 (0.2%) |
| Ligament sprain | 0 | 0 | 0 | 1 (0.9%) | 1 (0.2%) |
| Immune system disorders | 0 | 1 (1.2%) | 0 | 0 | 1 (0.2%) |
| Seasonal allergy | 0 | 1 (1.2%) | 0 | 0 | 1 (0.2%) |
| Metabolism and nutrition disorders | 1 (0.7%) | 0 | 0 | 0 | 1 (0.2%) |
| Dehydration | 1 (0.7%) | 0 | 0 | 0 | 1 (0.2%) |
| Neoplasms benign, malignant and | 1 (0.7%) | 0 | 0 | 0 | 1 (0.2%) |
| unspecified (incl cysts and polyps) | | | | | |
| Pyogenic granuloma | 1 (0.7%) | 0 | 0 | 0 | 1 (0.2%) |

Note:
Only treatment-emergent AEs are included in the table. AEs are coded using MedDRA v22.0.
Note:
At each level of summation (overall, system organ class, preferred term), subjects reporting more than one AE are counted only once.
Note:
System organ classes are listed in descending order based on the total column; within each system organ class, preferred terms are listed in descending order based on the total column.
Note:
AE = Adverse Event; TEAE = Treatment-Emergent Adverse Event; MedDRA = Medical Dictionary for Regulatory Activities.
Note:
If the event was not coded, verbatim text was used for the preferred term.

TABLE 51C
Treatment-Emergent Adverse Events by Severity, System Organ Class, and Preferred Term-Safety

| System | FCD105 | | | Vehicle | | | Minocycline 3% | | | Adapalene 0.3% | | |
|---|---|---|---|---|---|---|---|---|---|---|---|---|
| Organ Class/ | (N = 142) | | | (N = 82) | | | (N = 110) | | | (N = 112) | | |
| Preferred Term | Mild | Moderate | Severe | Mild | Moderate | Severe | Mild | Moderate | Severe | Mild | Moderate | Severe |
| Subjects | 12 (8.5%) | 9 (6.3%) | 0 | 7 (8.5%) | 3 (3.7%) | 0 | 13 (11.8%) | 2 (1.8%) | 0 | 14 (12.5%) | 4 (3.6%) | 2 (1.8%) |
| reporting at | | | | | | | | | | | | |
| least one TEAE | | | | | | | | | | | | |
| Infections and | 5 (3.5%) | 2 (1.4%) | 0 | 4 (4.9%) | 3 (3.7%) | 0 | 8 (7.3%) | 1 (0.9%) | 0 | 5 (4.5%) | 3 (2.7%) | 0 |
| infestations | | | | | | | | | | | | |
| Upper respiratory | 1 (0.7%) | 1 (0.7%) | 0 | 3 (3.7%) | 1 (1.2%) | 0 | 3 (2.7%) | 0 | 0 | 1 (0.9%) | 0 | 0 |
| tract infection | | | | | | | | | | | | |
| Nasopharyngitis | 2 (1.4%) | 0 | 0 | 0 | 0 | 0 | 3 (2.7%) | 0 | 0 | 0 | 0 | 0 |
| Sinusitis | 1 (0.7%) | 0 | 0 | 0 | 0 | 0 | 2 (1.8%) | 0 | 0 | 1 (0.9%) | 0 | 0 |
| Viral upper | 0 | 0 | 0 | 1 (1.2%) | 1 (1.2%) | 0 | 0 | 0 | 0 | 0 | 1 (0.9%) | 0 |
| respiratory | | | | | | | | | | | | |
| tract infection | | | | | | | | | | | | |
| Influenza | 0 | 1 (0.7%) | 0 | 0 | 0 | 0 | 0 | 0 | 0 | 0 | 1 (0.9%) | 0 |
| Pharyngitis | 0 | 0 | 0 | 0 | 1 (1.2%) | 0 | 0 | 1 (0.9%) | 0 | 0 | 0 | 0 |
| streptococcal | | | | | | | | | | | | |
| Acute sinusitis | 0 | 0 | 0 | 0 | 0 | 0 | 0 | 0 | 0 | 1 (0.9%) | 0 | 0 |
| Hordeolum | 0 | 0 | 0 | 0 | 0 | 0 | 0 | 0 | 0 | 1 (0.9%) | 0 | 0 |
| Infectious | 0 | 0 | 0 | 0 | 1 (1.2%] | 0 | 0 | 0 | 0 | | 0 | 0 |
| mononucleosis | | | | | | | | | | | | |
| Otitis media acute | 1 (0.7%) | 0 | 0 | 0 | 0 | 0 | 0 | 0 | 0 | 0 | 0 | 0 |
| Pharyngitis | 0 | 0 | 0 | 0 | 0 | 0 | 0 | 0 | 0 | 1 (0.9%) | 0 | 0 |
| Staphylococcal | 0 | 0 | 0 | 0 | 0 | 0 | 0 | 0 | 0 | 0 | 1 (0.9%) | 0 |
| infection | | | | | | | | | | | | |
| Skin and | 6 (4.2%) | 0 | 0 | 0 | 0 | 0 | 3 (2.7%) | 0 | 0 | 7 (6.3%) | 1 (0.9%) | 2 (1.8%) |
| subcutaneous | | | | | | | | | | | | |
| tissue disorders | | | | | | | | | | | | |
| Dry skin | 2 (1.4%) | 0 | 0 | 0 | 0 | 0 | 1 (0.9%) | 0 | 0 | 3 (2.7%) | 1 (0.9%) | 0 |
| Rash | 2 (1.4%) | 0 | 0 | 0 | 0 | 0 | 1 (0.9%) | 0 | 0 | 2 (1.8%) | 0 | 0 |
| Acne | 1 (0.7%) | 0 | 0 | 0 | 0 | 0 | 0 | 0 | 0 | 0 | 0 | 2 (1.8%) |
| Dermatitis | 1 (0.7%) | 0 | 0 | 0 | 0 | 0 | 0 | 0 | 0 | 0 | 0 | 0 |
| contact | | | | | | | | | | | | |
| Eczema | 0 | 0 | 0 | 0 | 0 | 0 | 0 | 0 | 0 | 1 (0.9%) | 0 | 0 |
| Nail | 0 | 0 | 0 | 0 | 0 | 0 | 1 (0.9%) | 0 | 0 | 0 | 0 | 0 |
| discolouration | | | | | | | | | | | | |
| Pain of skin | 1 (0.7%) | 0 | 0 | 0 | 0 | 0 | 0 | 0 | 0 | 0 | 0 | 0 |
| Skin | 0 | 0 | 0 | 0 | 0 | 0 | 0 | 0 | 0 | 1 (0.9%) | 0 | 0 |
| discolouration | | | | | | | | | | | | |
| Skin irritation | 0 | 0 | 0 | 0 | 0 | 0 | 0 | 0 | 0 | 1 (0.9%) | 0 | 0 |
| Nervous system | 2 (1.4%) | 2 (1,4%) | 0 | 3 (3.7%) | 0 | 0 | 2 (1.8%) | 0 | 0 | 2 (1.8%) | 0 | 0 |
| disorders | | | | | | | | | | | | |
| Headache | 1 (0.7%) | 0 | 0 | 3 (3.7%) | 0 | 0 | 2 (1.8%) | 0 | 0 | 2 (1.8%) | 0 | 0 |
| Buming sensation | 0 | 1 (0.7%) | 0 | 0 | 0 | 0 | 0 | 0 | 0 | 0 | 0 | 0 |
| Hyperaesthesia | 1 (0.7%) | 0 | 0 | 0 | 0 | 0 | 0 | 0 | 0 | 0 | 0 | 0 |
| Sciatica | 0 | 1 (0.7%) | 0 | 0 | 0 | 0 | 0 | 0 | 0 | 0 | 0 | 0 |
| Gastrointestinal | 2 (1.4%) | 3 (2.1%) | 0 | 1 (1.2%) | 0 | 0 | 1 (0.9%) | 0 | 0 | 1 (0.9%) | 0 | 0 |
| disorders | | | | | | | | | | | | |
| Diarrhoea | 0 | 1 (0.7%) | 0 | 1 (1.2%) | 0 | 0 | 0 | 0 | 0 | 1 (0.9%) | 0 | 0 |
| Vomiting | 0 | 2 (1,4%) | 0 | 0 | 0 | 0 | 1 (0.9%) | 0 | 0 | 0 | 0 | 0 |
| Nausea | 0 | 1 (0.7%) | 0 | 0 | 0 | 0 | 1 (0.9%) | 0 | 0 | 0 | 0 | 0 |
| Abdominal pain | 1 (0.7%) | 0 | 0 | 0 | 0 | 0 | 0 | 0 | 0 | 0 | 0 | 0 |
| Constipation | 0 | 1 (0.7%) | 0 | 0 | 0 | 0 | 0 | 0 | 0 | 0 | 0 | 0 |
| Toothache | 1 (0.7%) | 0 | 0 | 0 | 0 | 0 | 0 | 0 | 0 | 0 | 0 | 0 |
| Respiratory, | 0 | 0 | 0 | 0 | 0 | 0 | 2 (1.8%) | 1 (0.9%) | 0 | 1 (0.9%) | 0 | 0 |
| thoracic and | | | | | | | | | | | | |
| mediastinal | | | | | | | | | | | | |
| disorders | | | | | | | | | | | | |
| Cough | 0 | 0 | 0 | 0 | 0 | 0 | 0 | 0 | 0 | 1 (0.9%) | 0 | 0 |
| Oropharyngeal | 0 | 0 | 0 | 0 | 0 | 0 | 1 (0.9%) | 0 | 0 | 0 | 0 | 0 |
| pain | | | | | | | | | | | | |
| Paranasal sinus | 0 | 0 | 0 | 0 | 0 | 0 | 0 | 1 (0.9%) | 0 | 0 | 0 | 0 |
| discomfort | | | | | | | | | | | | |
| Rhinorrhoea | 0 | 0 | 0 | 0 | 0 | 0 | 1 (0.9%) | 0 | 0 | 0 | 0 | 0 |
| Eye disorders | 0 | 0 | 0 | 0 | 0 | 0 | 0 | 0 | 0 | 3 (2.7%) | 0 | 0 |
| Eye irritation | 0 | 0 | 0 | 0 | 0 | 0 | 0 | 0 | 0 | 2 (1.8%) | 0 | 0 |
| Erythema | 0 | 0 | 0 | 0 | 0 | 0 | 0 | 0 | 0 | 1 (0.9%) | 0 | 0 |
| of eyelid | | | | | | | | | | | | |
| General | 0 | 1 (0.7%) | 0 | 0 | 0 | 0 | 1 (0.9%) | 0 | 0 | 1 (0.9%) | 0 | 0 |
| disorders and | | | | | | | | | | | | |
| administration | | | | | | | | | | | | |
| site conditions | | | | | | | | | | | | |
| Influenza like | 0 | 1 (0.7%) | 0 | 0 | 0 | 0 | 0 | 0 | 0 | 1 (0.9%) | 0 | 0 |
| illness | | | | | | | | | | | | |
| Pain | 0 | 0 | 0 | 0 | 0 | 0 | 1 (0.9%) | 0 | 0 | 0 | 0 | 0 |
| Injury, poisoning | 0 | 1 (0.7%) | 0 | 0 | 0 | 0 | 0 | 0 | 0 | 2 (1.8%) | 0 | 0 |
| and procedural | | | | | | | | | | | | |
| complications | | | | | | | | | | | | |
| Burns first | 0 | 0 | 0 | 0 | 0 | 0 | 0 | 0 | 0 | 1 (0.9%) | 0 | 0 |
| degree | | | | | | | | | | | | |
| Facial bones | 0 | 0 | 0 | 0 | 0 | 0 | 0 | 0 | 0 | 1 (0.9%) | 0 | 0 |
| fracture | | | | | | | | | | | | |
| Ligament rupture | 0 | 1 (0.7%) | 0 | 0 | 0 | 0 | 0 | 0 | 0 | 0 | 0 | 0 |
| Ligament sprain | 0 | 0 | 0 | 0 | 0 | 0 | 0 | 0 | 0 | 1 (0.9%) | 0 | 0 |
| Immune system | 0 | 0 | 0 | 1 (1.2%) | 0 | 0 | 0 | 0 | 0 | 0 | 0 | 0 |
| disorders | | | | | | | | | | | | |
| Seasonal allergy | 0 | 0 | 0 | 1 (1.2%) | 0 | 0 | 0 | 0 | 0 | 0 | 0 | 0 |
| Metabolism and | 0 | 1 (0.7%) | 0 | 0 | 0 | 0 | 0 | 0 | 0 | 0 | 0 | 0 |
| nutrition | | | | | | | | | | | | |
| disorders | | | | | | | | | | | | |
| Dehydration | 0 | 1 (0.7%) | 0 | 0 | 0 | 0 | 0 | 0 | 0 | 0 | 0 | 0 |
| Neoplasms | 1 (0.7%) | 0 | 0 | 0 | 0 | 0 | 0 | 0 | 0 | 0 | 0 | 0 |
| benign, | | | | | | | | | | | | |
| malignant | | | | | | | | | | | | |
| and unspecified | | | | | | | | | | | | |
| (incl | | | | | | | | | | | | |
| cysts and polyps) | | | | | | | | | | | | |
| Pyogenic | 1 (0.7%) | 0 | 0 | 0 | 0 | 0 | 0 | 0 | 0 | 0 | 0 | 0 |
| granuloma | | | | | | | | | | | | |

Note:
Only treatment-emergent AEs are included in the table. AEs are coded using MedDRA v22.0.
Note:
At each level of summation (overall, system organ class, preferred term), subjects reporting more than one AE are counted only once.
Note:
System organ classes are listed in descending order based on the total column; within each system organ class, preferred terms are listed in descending order based on the total column.
Note:
AE = Adverse Event;
TEAE = Treatment-Emergent Adverse Event;
MedDRA = Medical Dictionary for Regulatory Activities.
Note:
If the event was not coded, verbatim text was used for the preferred term.

TABLE 51D
Treatment-Related Adverse Events by System Organ Class and Preferred Term - Safety Population

| System Organ Class/ | FCD105 | Vehicle | Minocycline 3% | Adapalene 0.3% | Total |
|---|---|---|---|---|---|
| Preferred Term | (N = 142) | (N = 82) | (N = 110) | (N = 112) | (N = 446) |
| Subjects reporting at least one | 5 (3.5%) | 0 | 2 (1.8%) | 10 (8.9%) | 17 (3.8%) |
| treatment-related TEAE | | | | | |
| Skin and subcutaneous tissue disorders | 4 (2.8%) | 0 | 2 (1.8%) | 9 (8.0%) | 15 (3.4%) |
| Dry skin | 1 (0.7%) | 0 | 1 (0.9%) | 4 (3.6%) | 6 (1.3%) |
| Rash | 1 (0.7%) | 0 | 0 | 2 (1.8%) | 3 (0.7%) |
| Acne | 0 | 0 | 0 | 2 (1.8%) | 2 (0.4%) |
| Dermatitis contact | 1 (0.7%) | 0 | 0 | 0 | 1 (0.2%) |
| Nail discolouration | 0 | 0 | 1 (0.9%) | 0 | 1 (0.2%) |
| Pain of skin | 1 (0.7%) | 0 | 0 | 0 | 1 (0.2%) |
| Skin discolouration | 0 | 0 | 0 | 1 (0.9%) | 1 (0.2%) |
| Skin irritation | 0 | 0 | 0 | 1 (0.9%) | 1 (0.2%) |
| Eye disorders | 0 | 0 | 0 | 3 (2.7%) | 3 (0.7%) |
| Eye irritation | 0 | 0 | 0 | 2 (1.8%) | 2 (0.4%) |
| Erythema of eyelid | 0 | 0 | 0 | 1 (0.9%) | 1 (0.2%) |
| Nervous system disorders | 2 (1.4%) | 0 | 0 | 0 | 2 (0.4%) |
| Burning sensation | 1 (0.7%) | 0 | 0 | 0 | 1 (0.2%) |
| Hyperaesthesia | 1 (0.7%) | 0 | 0 | 0 | 1 (0.2%) |

Note:
Only treatment-emergent AEs are included in the table. AEs are coded using MedDRA v22.0.
Note:
At each level of summation (overall, system organ class, preferred term), subjects reporting more than one AE are counted only once.
Note:
System organ classes are listed in descending order based on the total column; within each system organ class, preferred terms are listed in descending order based on the total column.
Note:
AE = Adverse Event; TEAE = Treatment-Emergent Adverse Event; MedDRA = Medical Dictionary for Regulatory Activities.
Note:
If the event was not coded, verbatim text was used for the preferred term.

TABLE 51E
Serious Adverse Events by System Organ Class
and Preferred Term - Safety Population

No data meets criteria.

TABLE 51F
Serious Adverse Events by Severity, System Organ
Class, and Preferred Term - Safety Population

No data meets criteria.

TABLE 51G
Treatment-Related Serious Adverse Events by System
Organ Class and Preferred Term - Safety Population

No data meets criteria.

TABLE 51H
Adverse Events Leading to Withdrawal from the Study by System
Organ Class and Preferred Term - Safety Population

| System Organ Class/ | FCD105 | Vehicle | Minocycline 3% | Adapalene 0.3% | Total |
|---|---|---|---|---|---|
| Preferred Term | (N = 142) | (N = 82) | (N = 110) | (N = 112) | (N = 446) |
| Number (%) of subjects withdrawn from | 1 (0.7%) | 0 | 0 | 3 (2.7%) | 4 (0.9%) |
| study due to TEAE | | | | | |
| Skin and subcutaneous tissue disorders | 1 (0.7%) | 0 | 0 | 3 (2.7%) | 4 (0.9%) |
| Acne | 1 (0.7%) | 0 | 0 | 2 (1.8%) | 3 (0.7%) |
| Rash | 0 | 0 | 0 | 1 (0.9%) | 1 (0.2%) |

Note:
Only treatment-emergent AEs are included in the table. AEs are coded using MedDRA v22.0.
Note:
At each level of summation (overall, system organ class, preferred term), subjects reporting more than one AE are counted only once.
Note:
System organ classes are listed in descending order based on the total column; within each system organ class, preferred terms are listed in descending order based on the total column.
Note:
AE = Adverse Event; TEAE = Treatment-Emergent Adverse Event; MedDRA = Medical Dictionary for Regulatory Activities.
Note:
If the event was not coded, verbatim text was used for the preferred term.

TABLE 51I
Adverse Events Leading to Withdrawal from the Study by Severity, System Organ Class, and Preferred
Term-Safety Population

| | FCD105 | | | Vehicle | | | Minocycline 3% | | | Adapalene 0.3% | | |
|---|---|---|---|---|---|---|---|---|---|---|---|---|
| System Organ Class/ | (N = 142) | | | (N = 82) | | | (N = 110) | | | (N = 112) | | |
| Preferred Term | Mild | Moderate | Severe | Mild | Moderate | Severe | Mild | Moderate | Severe | Mild | Moderate | Severe |
| Number (%) of | 1 | 0 | 0 | 0 | 0 | 0 | 0 | 0 | 0 | 1 | 0 | 2 |
| subjects withdrawn | | | | | | | | | | | | |
| from study due to TEAE | (0.7%) | | | | | | | | | (0.9%) | | (1.8%) |
| Skin and subcutaneous | 1 | 0 | 0 | 0 | 0 | 0 | 0 | 0 | 0 | 1 | 0 | 2 |
| tissue disorders | (0.7% | | | | | | | | | (0.9%) | | (1.8%) |
| Acne | 1 | 0 | 0 | 0 | 0 | 0 | 0 | 0 | 0 | 0 | 0 | 2 |
| | (0.7%) | | | | | | | | | | | (1.8%) |
| Rash | 0 | 0 | 0 | 0 | 0 | 0 | 0 | 0 | 0 | 1 | 0 | 0 |
| | | | | | | | | | | (0.9%) | | |

Note:
Only treatment-emergent AEs are included in the table. AEs are coded using MedDRA v22.0.
Note:
At each level of summation (overall, system organ class, preferred term), subjects reporting more than one AE are counted only once.
Note:
System organ classes are listed in descending order based on the total column; within each system organ class, preferred terms are listed in descending
order based on the total column.
Note:
AE = Adverse Event; TEAE = Treatment-Emergent Adverse Event; MedDRA = Medical Dictionary for Regulatory Activities.
Note:
If the event was not coded, verbatim text was used for the preferred term.

TABLE 51J
Treatment-Related Adverse Events Leading to Withdrawal from the Study
by Systemic Organ Class, and Preferred Term - Safety Population

| System Organ Class/ | FCD105 | Vehicle | Minocycline 3% | Adapalene 0.3% | Total |
|---|---|---|---|---|---|
| Preferred Term | (N = 142) | (N = 82) | (N = 110) | (N = 112) | (N = 446) |
| Number (%) of subjects withdrawn from study | 0 | 0 | 0 | 3 (2.7%) | 3 (0.7%) |
| due to treatment-related TEAE | | | | | |
| Skin and subcutaneous tissue disorders | 0 | 0 | 0 | 3 (2.7%) | 3 (0.7%) |
| Acne | 0 | 0 | 0 | 2 (1.8%) | 2 (0.4%) |
| Rash | 0 | 0 | 0 | 1 (0.9%) | 1 (0.2%) |

Note:
Only treatment-emergent AEs are included in the table. AEs are coded using MedDRA v22.0.
Note:
At each level of summation (overall, system organ class, preferred term), subjects reporting more than one AE are counted only once.
Note:
System organ classes are listed in descending order based on the total column; within each system organ class, preferred terms are listed in descending order based on the total column.
Note:
AE = Adverse Event; TEAE = Treatment-Emergent Adverse Event; MedDRA = Medical Dictionary for Regulatory Activities.
Note:
If the event was not coded, verbatim text was used for the preferred term.

TABLE 52
Shift Table of Physical Examination Results-Safety Population

| | FCD105 | | Vehicle | | Minocydine 3% | | Adapalene 0.3% | |
|---|---|---|---|---|---|---|---|---|
| | (N = 142) | | (N = 82) | | (N = 110) | | (N = 112) | |
| | Baseline | | Baseline | | Baseline | | Baseline | |
| | Normal | Abnormal | Normal | Abnormal | Normal | Abnormal | Normal | Abnormal |
| Week 12 | | | | | | | | |
| Normal | 118 (83.1%) | 6 (4.2%) | 64 (78.0%) | 2 (2.4%) | 94 (85.5%) | 3 (2.7%) | 86 (76.8%) | 4 (3.6%) |
| Abnormal | 2 (1.4%) | 2 (1.4%) | 6 (7.3%) | 4 (4.9%) | 3 (2.7%) | 3 (2.7%) | 4 (3.6%) | 5 (4.5%) |

TABLE 53
Vital Sign Results - Safety Population

| Parameter | | FCD105 | | Vehicle | | Minocycline 3% | | Adapalene 0.3% | |
|---|---|---|---|---|---|---|---|---|---|
| Visit | Statistics | (N = 142) | | (N = 82) | | (N = 110) | | (N = 112) | |
| Heart Rate | | | | | | | | | |
| (beats/min) | | | | | | | | | |
| Baseline | n | 142 | | 82 | | 110 | | 112 | |
| | Mean (SD) | 74.8 | (10.03) | 73.4 | (8.85) | 72.4 | (10.70) | 74.1 | (10.53) |
| | Median | 74.0 | | 72.0 | | 71.5 | | 73.0 | |
| | Min, Max | 49, 99 | | 56, 92 | | 51, 106 | | 52, 100 | |
| Week 4 | | 135 | | 82 | | 106 | | 103 | |
| | Mean (SD) | 75.6 | (11.71) | 73.8 | ( 10.71) | 75.5 | (10.72) | 75.9 | (9.91) |
| | Median | 74.0 | | 73.0 | | 75.0 | | 77.0 | |
| | Min, Max | 52, 111 | | 51, 111 | | 52, 107 | | 52, 99 | |
| Week 8 | | 128 | | 78 | | 101 | | 105 | |
| | Mean (SD) | 76.8 | (9.29) | 74.2 | (9.07) | 75.0 | (10.73) | 76.1 | (9.41) |
| | Median | 77.0 | | 73.0 | | 74.0 | | 77.0 | |
| | Min, Max | 52, 102 | | 58, 96 | | 52, 100 | | 51, 99 | |
| Week 12 | n | 128 | | 76 | | 104 | | 99 | |
| | Mean (SD) | 77.4 | (9.31) | 73.9 | (9.91) | 74.1 | (9.60) | 75.7 | (9.13) |
| | Median | 77.5 | | 72.5 | | 74.0 | | 76.0 | |
| | Min, Max | 56, 106 | | 54, 98 | | 50, 106 | | 52, 96 | |
| Systolic Blood | | | | | | | | | |
| Pressure (mmHg) | | | | | | | | | |
| Baseline | n | 142 | | 82 | | 110 | | 112 | |
| | Mean (SD) | 115.1 | (11.30) | 115.1 | (10.14) | 115.9 | (11.29) | 115.4 | (11.19) |
| | Median | 115.0 | | 116.0 | | 117.0 | | 115.0 | |
| | Min, Max | 87, 140 | | 90, 139 | | 82, 159 | | 92, 150 | |
| Week 4 | | 135 | | 82 | | 106 | | 103 | |
| | Mean (SD) | 116.5 | (11.22) | 116.7 | (11.80) | 117.1 | (10.75) | 115.9 | (10.19) |
| | Median | 118.0 | | 117.0 | | 118.0 | | 116.0 | |
| | Min, Max | 92, 148 | | 83, 143 | | 95, 144 | | 96, 150 | |
| Week 8 | | 128 | | 78 | | 101 | | 105 | |
| | Mean (SD) | 116.4 | (10.19) | 116.8 | (11.03) | 116.5 | (9.91) | 116.7 | (11.26) |
| | Median | 118.0 | | 117.0 | | 118.0 | | 115.0 | |
| | Min, Max | 83, 142 | | 95, 145 | | 91, 148 | | 95, 152 | |
| Week 12 | n | 128 | | 76 | | 104 | | 99 | |
| | Mean (SD) | 116.0 | (11.64) | 117.1 | (10.94) | 116.3 | (10.62) | 116.3 | (11.05) |
| | Median | 116.0 | | 116.0 | | 116.0 | | 115.0 | |
| | Min, Max | 84, 147 | | 94, 147 | | 87, 144 | | 92, 149 | |
| Diastolic Blood | | | | | | | | | |
| Pressure (mmHg) | | | | | | | | | |
| Baseline | | 142 | | 82 | | 110 | | 112 | |
| | Mean (SD) | 72.7 | (8.87) | 72.5 | (7.98) | 73.0 | (8.38) | 73.1 | (7.90) |
| | Median | 72.0 | | 72.0 | | 73.0 | | 72.0 | |
| | Min, Max | 54, 98 | | 50, 91 | | 50, 94 | | 58, 98 | |
| Week 4 | | 135 | | 82 | | 106 | | 103 | |
| | Mean (SD) | 73.5 | (8.89) | 73.5 | (7.52) | 73.8 | (7.64) | 72.8 | (6.91) |
| | Median | 73.0 | | 74.0 | | 74.5 | | 72.0 | |
| | Min, Max | 54, 92 | | 54, 87 | | 53, 94 | | 56, 93 | |
| Week 8 | n | 128 | | 78 | | 101 | | 105 | |
| | Mean (SD) | 74.3 | (7.62) | 72.0 | (7.36) | 72.5 | (8.77) | 72.1 | (8.23) |
| | Median | 74.5 | | 71.0 | | 71.0 | | 72.0 | |
| | Min, Max | 54, 94 | | 58, 88 | | 55, 100 | | 57, 94 | |
| Week 12 | n | 128 | | 76 | | 104 | | 99 | |
| | Mean (SD) | 73.7 | (8.30) | 72.9 | (8.06) | 73.2 | (8.62) | 72.7 | (8.02) |
| | Median | 73.0 | | 71.0 | | 72.5 | | 72.0 | |
| | Min, Max | 53, 96 | | 58, 93 | | 53, 96 | | 48, 94 | |
| Weight (kg) | | | | | | | | | |
| Baseline | n | 142 | | 82 | | 110 | | 112 | |
| | Mean (SD) | 72.68 | (21.322) | 75.18 | (21.516) | 69.93 | (16.194) | 77.06 | (24.619) |
| | Median | 66.10 | | 70.25 | | 67.15 | | 71.10 | |
| | Min, Max | 37.5, 138.8 | | 40.8, 133.4 | | 40.4, 110.7 | | 43.5, 163.3 | |
| Week 12 | n | 128 | | 76 | | 103 | | 99 | |
| | Mean (SD) | 72.67 | (20.987) | 74.70 | (20.763) | 70.12 | (16.675 | 77.28 | (24.907) |

| | | | | |
|---|---|---|---|---|
| Median | 66.60 | 69.90 | 66.00 | 71.50 |
| Min, Max | 37.5, 130.2 | 40.4, 131.5 | 40.5, 110.7 | 43.5, 166.0 |

TABLE 54
Summary of Local Signs and Symptoms Assessments - Safety Population

| | | | | |
|---|---|---|---|---|
| Parameter/ | FCD105 | Vehicle | Minocycline 3% | Adapalene 0.3% |
| Visit | (N = 142) | (N = 82) | (N = 110) | (N = 112) |

Burning and Stinging

| | | | | | |
|---|---|---|---|---|---|
| Baseline | None | 136 (96.5%) | 81 (98.8%) | 104 (94.5%) | 105 (93.8%) |
| | Mild | 2 (1.4%) | 0 | 5 (4.5%) | 7 (6.3%) |
| | Moderate | 3 (2.1%) | 1 (1.2%) | 1 (0.9%) | 0 |
| | Severe | 0 | 0 | 0 | 0 |
| Week 4 | None | 115 (85.8%) | 78 (95.1%) | 99 (93.4%) | 82 (78.1%) |
| | Mild | 18 (13.4%) | 4 (4.9%) | 7 (6.6%) | 20 (19.0%) |
| | Moderate | 1 (0.7%) | 0 | 0 | 3 (2.9%) |
| | Severe | 0 | 0 | 0 | 0 |
| Week 8 | None | 116 (90.6%) | 75 (96.2%) | 95 (94.1%) | 97 (92.4%) |
| | Mild | 10 (7.8%) | 2 (2.6%) | 5 (5.0%) | 7 (6.7%) |
| | Moderate | 2 (1.6%) | 1 (1.3%) | 1 (1.0%) | 1 (1.0%) |
| | Severe | 0 | 0 | 0 | 0 |
| Week 12 | None | 124 (96.9%) | 72 (94.7%) | 99 (95.2%) | 89 (89.9%) |
| | Mild | 3 (2.3%) | 4 (5.3%) | 3 (2.9%) | 8 (8.1%) |
| | Moderate | 1 (0.8%) | 0 | 2 (1.9%) | 2 (2.0%) |
| | Severe | 0 | 0 | 0 | 0 |

Itching

| | | | | | |
|---|---|---|---|---|---|
| Baseline | None | 114 (80.9%) | 70 (85.4%) | 93 (84.5%) | 87 (77.7%) |
| | Mild | 24 (17.0%) | 11 (13.4%) | 14 (12.7%) | 22 (19.6%) |
| | Moderate | 2 (1.4%) | 1 (1.2%) | 3 (2.7%) | 2 (1.8%) |
| | Severe | 1 (0.7%) | 0 | 0 | 1 (0.9%) |
| Week 4 | None | 120 (89.6%) | 75 (91.5%) | 95 (89.6%) | 79 (75.2%) |
| | Mild | 13 (9.7%) | 7 (8.5%) | 9 (8.5%) | 22 (21.0%) |
| | Moderate | 1 (0.7%) | 0 | 2 (1.9%) | 4 (3.8%) |
| | Severe | 0 | 0 | 0 | 0 |
| Week 8 | None | 112 (87.5%) | 69 (88.5%) | 88 (87.1%) | 86 (81.9%) |
| | Mild | 15 (11.7%) | 7 (9.0%) | 12 (11.9%) | 18 (17.1%) |
| | Moderate | 1 (0.8%) | 2 (2.6%) | 1 (1.0%) | 1 (1.0%) |
| | Severe | 0 | 0 | 0 | 0 |
| Week 12 | None | 116 (90.6%) | 68 (89.5%) | 94 (90.4%) | 86 (86.9%) |
| | Mild | 10 (7.8%) | 7 (9.2%) | 9 (8.7%) | 10 (10.1%) |
| | Moderate | 2 (1.6%) | 1 (1.3%) | 1 (1.0%) | 3 (3.0%) |
| | Severe | 0 | 0 | 0 | 0 |

Dryness

| | | | | | |
|---|---|---|---|---|---|
| Baseline 1 | None | 101 (72.1%) | 53 (64.6%) | 81 (73.6%) | 82 (73.2%) |
| | Mild | 34 (24.3%) | 28 (34.1%) | 26 (23.6%) | 27 (24.1%) |
| | Moderate | 5 (3.6%) | 1 (1.2%) | 3 (2.7%) | 3 (2.7%) |
| | Severe | 0 | 0 | 0 | 0 |
| Week 4 | None | 107 (79.9%) | 59 (72.0%) | 86 (81.1%) | 75 (71.4%) |
| | Mild | 24 (17.9%) | 22 (26.8%) | 18 (17.0%) | 26 (24.8%) |
| | Moderate | 3 (2.2%) | 1 (1.2%) | 2 (1.9%) | 4 (3.8%) |
| | Severe | 0 | 0 | 0 | 0 |
| Week 8 | None | 96 (75.0%) | 59 (75.6%) | 84 (83.2%) | 77 (73.3%) |
| | Mild | 30 (23.4%) | 18 (23.1%) | 14 (13.9%) | 26 (24.8%) |
| | Moderate | 2 (1.6%) | 1 (1.3%) | 3 (3.0%) | 2 (1.9%) |
| | Severe | 0 | 0 | 0 | 0 |
| Week 12 | None | 111 (86.7%) | 59 (77.6%) | 91 (87.5%) | 81 (81.8%) |
| | Mild | 15 (11.7%) | 16 (21.1%) | 12 (11.5%) | 17 (17.2%) |
| | Moderate | 2 (1.6%) | 1 (1.3%) | 1 (1.0%) | 1 (1.0%) |
| | Severe | 0 | 0 | 0 | 0 |

Scaling

| | | | | | |
|---|---|---|---|---|---|
| Baseline | None | 120 (85.1%) | 62 (75.6%) | 91 (82.7%) | 89 (79.5%) |
| | Mild | 16 (11.3%) | 17 (20.7%) | 18 (16.4%) | 21 (18.8%) |
| | Moderate | 5 (3.5%) | 3 (3.7%) | 1 (0.9%) | 2 (1.8%) |
| | Severe | 0 | 0 | 0 | 0 |
| Meek 4 | None | 116 (86.6%) | 66 (80.5%) | 95 (89.6%) | 83 (79.0%) |
| | Mild | 17 (12.7%) | 14 (17.1%) | 9 (8.5%) | 19 (18.1%) |
| | Moderate | 1 (0.7%) | 2 (2.4%) | 2 (1.9%) | 3 (2.9%) |
| | Severe | 0 | 0 | 0 | 0 |
| Week 8 | None | 108 (84.4%) | 65 (83.3%) | 92 (91.1%) | 86 (81.9%) |
| | Mild | 18 (14.1%) | 12 (15.4%) | 9 (8.9%) | 17 (16.2%) |
| | Moderate | 2 (1.6%) | 1 (1.3%) | 0 | 2 (1.9%) |
| | Severe | 0 | 0 | 0 | 0 |
| Week 12 | None | 115 (89.8%) | 66 (86.8%) | 99 (95.2%) | 84 (84.8%) |
| | Mild | 12 (9.4%) | 9 (11.8%) | 5 (4.8%) | 12 (12.1%) |
| | Moderate | 1 (0.8%) | 1 (1.3%) | 0 | 3 (3.0%) |
| | Severe | 0 | 0 | 0 | 0 |

Erythema

| | | | | | |
|---|---|---|---|---|---|
| Baseline | None | 79 (56.0%) | 41 (50.0%) | 64 (58.2%) | 51 (45.5%) |
| | Mild | 39 (27.7%) | 26 (31.7%) | 23 (20.9%) | 36 (32.1%) |
| | Moderate | 21 (14.9%) | 15 (18.3%) | 22 (20.0%) | 24 (21.4%) |
| | Severe | 2 (1.4%) | 0 | 1 (0.9%) | 1 (0.9%) |
| Week 4 | None | 78 (58.2%) | 49 (59.8%) | 68 (64.2%) | 55 (52.4%) |
| | Mild | 39 (29.1%) | 25 (30.5%) | 22 (20.8%) | 27 (25.7%) |
| | Moderate | 15 (11.2%) | 8 (9.8%) | 16 (15.1%) | 21 (20.0%) |
| | Severe | 2 (1.5%) | 0 | 0 | 2 (1.9%) |
| Week 8 | None | 79 (61.7%) | 46 (59.0%) | 62 (61.4%) | 58 (55.2%) |
| | Mild | 37 (28.9%) | 29 (37.2%) | 32 (31.7%) | 36 (34.3%) |
| | Moderate | 10 (7.8%) | 3 (3.8%) | 7 (6.9%) | 10 (9.5%) |
| | 1 Severe | 2 (1.6%) | 0 | 0 | 1 (1.0%) |
| Week 12 | None | 89 (69.5%) | 49 (64.5%) | 70 (67.3%) | 62 (62.6%) |
| | Mild | 30 (23.4%) | 23 (30.3%) | 32 (30.8%) | 27 (27.3%) |
| | Moderate | 8 (6.3%) | 4 (5.3%) | 2 (1.9%) | 9 (9.1%) |
| | Severe | 1 (0.8%) | 0 | 0 | 1 (1.0%) |

Hyperpigmentation

| | | | | | |
|---|---|---|---|---|---|
| Baseline | None | 103 (73.0%) | 58 (70.7%) | 83 (75.5%) | 75 (67.0%) |
| | Mild | 33 (23.4%) | 16 (19.5%) | 22 (20.0%) | 31 (27.7%) |
| | Moderate | 5 (3.5%) | 8 (9.8%) | 5 (4.5%) | 6 (5.4%) |
| | Severe | 0 | 0 | 0 | 0 |
| Week 4 | None | 111 (82.8%) | 64 (78.0%) | 83 (78.3%) | 79 (75.2%) |
| | Mild | 18 (13.4%) | 12 (14.6%) | 20 (18.9%) | 22 (21.0%) |
| | Moderate | 5 (3.7%) | 6 (7.3%) | 3 (2.8%) | 4 (3.8%) |
| | Severe | 0 | 0 | 0 | 0 |
| Week 8 | None | 102 (79.7%) | 64 (82.1%) | 82 (81.2%) | 81 (77.1%) |
| | Mild | 21 (16.4%) | 9 (11.5%) | 16 (15.8%) | 20 (19.0%) |
| | Moderate | 5 (3.9%) | 5 (6.4%) | 3 (3.0%) | 4 (3.8%) |
| | Severe | 0 | 0 | 0 | 0 |
| Week 12 | None | 104 (81.3%) | 61 (80.3%) | 84 (80.8%) | 76 (76.8%) |
| | Mild | 20 (15.6%) | 10 (13.2%) | 17 (16.3%) | 19 (19.2%) |
| | Moderate | 4 (3.1%) | 5 (6.6%) | 3 (2.9%) | 4 (4.0%) |
| | Severe | 0 | 0 | 0 | 0 |

Note:
The denominator for the percentages is the number of observed cases in each treatment group.

### DISCUSSION AND OVERALL CONCLUSIONS

FCD105 was found to be significantly superior to vehicle with respect to absolute change from Baseline in inflammatory lesion counts and IGA treatment success after 12 weeks of treatment. Statistical significance was not reached for absolute change from Baseline in non-inflammatory lesion counts. These findings were supported by sensitivity analyses and the results of secondary efficacy analyses.

Secondary efficacy analyses compared FCD105 with the 2 individual ingredients, minocycline 3% and adapalene 0.3%. FCD105 was statistically superior to adapalene 0.3% in absolute change from Baseline in inflammatory and non-inflammatory lesion counts at Visit 4 (Week 12/End of Treatment). FCD105 was also statistically superior to minocycline 3% in absolute change from Baseline in non-inflammatory lesion counts at Visit 4, but this difference was not statistically significant for inflammatory lesion counts. For IGA treatment success, FCD105 was statistically superior to adapalene 0.3% but not to minocycline 3% at Visit 4.

Without being bond by any theory, these findings are in accordance with the fact that minocycline and adapalene are both effective against acne and act via different mechanisms, and thus, combination of both in FCD105 offers an advantage.

In agreement with the safety profiles available for minocycline 3% and adapalene 0.3% as individual components, FCD105 exhibited a favorable safety profile, with the majority of TEAEs being characterized as mild or moderate. Only 2 subjects experienced TEAEs that were considered severe. The primary treatment-related TEAEs included dry skin, rash, acne, and eye irritation, and only 4 subjects (1 subject in the FCD105 arm and 3 subjects in the adapalene 0.3% arm) experienced TEAEs that led to discontinuation of study drug.

### Example 32. Raman Spectroscopy Testing

### Part A

### Description

Two placebo batches of FCD105 (pre foam formulations) were evaluated by Raman spectroscopy: batch 190423 manufactured by continuous process (MCO process) and batch 191105S, manufactured by process with holding step of 4 hours at 54° C. (MAH process) (Table 55). The Raman spectra of the crystals present in these samples were collected and compared to detect any impact of different manufacturing processes.

TABLE 55
Formulations

| Formulation # | 190423 Vehicle | 1911105S Vehicle |
|---|---|---|
| HCO amount | 1.2% | 1.2% |
| API | No | No |
| Process | Continuous | Holding process |
| | heating-cooling | At 54° C. for four hours |
| Component | | |
| Soybean oil | 50.0 | 50.0 |
| Coconut oil | 23.6 | 23.6 |
| Light mineral oil | 6.6 | 6.6 |
| Stearic acid | 3.0 | 3.0 |
| Docosanol | 1.1 | 1.1 |
| Hydrogenated castor oil | 1.2 | 1.2 |
| White wax (beeswax) | 2.0 | 2.0 |
| Stearyl alcohol | 1.5 | 1.5 |
| Cetostearyl alcohol | 3.5 | 3.5 |
| Myristyl alcohol | 2.5 | 2.5 |
| Cyclomethicone | 5.0 | 5.0 |
| TOTAL | 100 | 100 |

### Methods

Stimulated Raman scattering (SRS) images and spectra were acquired on a Leica SP8 laser scanning microscope coupled to a PicoEmerald-S laser system for stimulated Raman scattering microscopy. The PicoEmerald-S outputs two pulsed 2 ps laser beams: a 1031 nm Stokes beam which is spatially and temporally overlapped with a tuneable pump beam. The Stokes beam is modulated at 20 MHz and stimulated Raman loss signals were detected using a lock-in amplifier (UHFLI, Zurich instruments) to measure modulation transfer onto the pump beam. A second channel was utilized to measure second harmonic and fluorescence emission signals. Samples were prepared by drop casting a small volume of formulation onto a coverslip and placing a second coverslip over the top, which was sealed prior to imaging. Images were acquired with a water immersion ×40 magnification lens (1.1 NA, Leica) used in conjunction with an oil immersion condenser lens (1.4 NA, Leica). Laser power was set to 30% which corresponds to approximately 10 mW for the pump beam and 30 mW for the Stokes beam at the sample. The data was acquired at ambient laboratory temperature. SRS data have been processed using Leica LAS-X software.

### Results

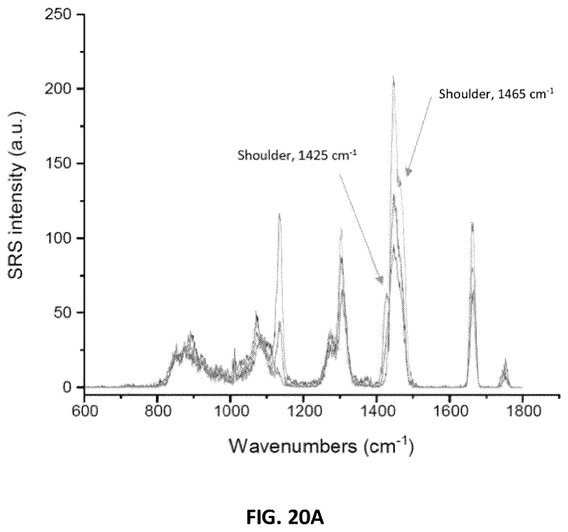

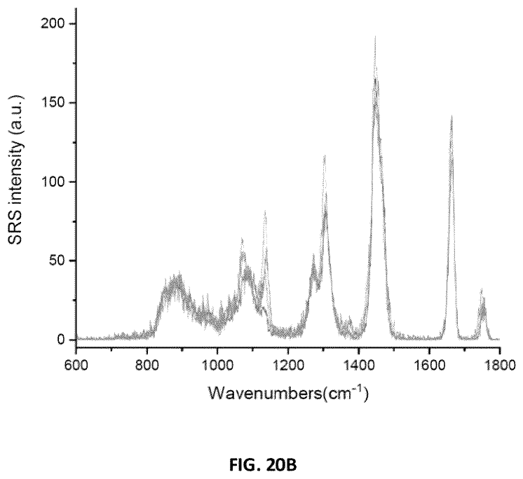

SRS spectra acquired for observed crystals (different regions of interest, or “ROI”) in the sample of batch 191105S are presented in FIG. 20A. Similarly, SRS spectra of batch 190423 are presented in FIG. 20B. In each one of FIGS. 20A and 20B, an overlay of four representative SRS spectra was presented, which corresponds to analysis of four different regions in the sample.

The Raman fingerprint detected in a formulation manufactured using a holding process (MAH process) is different to what is observed for a formulation prepared using a continuous process (MCO process). A difference was observed between samples prepared by a continuous process or a holding process for the peak at ˜1446 cm⁻¹. The peak at ˜1446 cm⁻¹ has distinct shoulder peaks (at 1425 cm⁻¹ and at ˜1465 cm⁻¹) for batch 191105S (MAH process), whereas these are absent or highly reduced for the 190423 batch (MCO process).

These results show that different crystalline structures, possessing characteristic Raman fingerprints, are detectable depending on whether the formulation is manufactured using a holding process (MAH process), or a contrasting continuous process (MCO process).

In one or more embodiments, formulations manufactured using a continuous process does not exhibit a peak having one or more shoulders in the range 1400-1500 cm⁻¹ of Raman spectrum.

### Part B

Formulations (Table 56) comprising soybean oil and HCO manufactured by continuous process (MCO process) and manufactured by process with holding step of 4 hours at 54° C. (MAH process) are evaluated by a Raman spectroscopy study.

TABLE 56
Formulations

| Formulation # | Vehicle | Vehicle |
|---|---|---|
| API | No | No |
| Process | Continuous | Holding process |
| | heating-cooling | At 54° C. for four hours |
| Component | | |
| Soybean oil | 98.2 | 98.2 |
| HCO | 1.2% | 1.2% |
| Total | 100 | 100 |

### Example 33. Effect of Shear on TMH Crystals and TM4 Temperature

### Part A

### Description

An initial exploratory study to evaluate the influence of shear on the formation and or elimination of the Tmh and other crystals was performed, and further testing is ongoing.

### Methods

Batches (see Table 57) were manufactured utilizing an ESCO 3L mixing unit (supplied by ESCO—Labor, Switzerland), into which ingredients were added. The unit was equipped with a homogenizer immersed into the ingredients. Various batches were made with alterations in the manufacturing process.

### Results

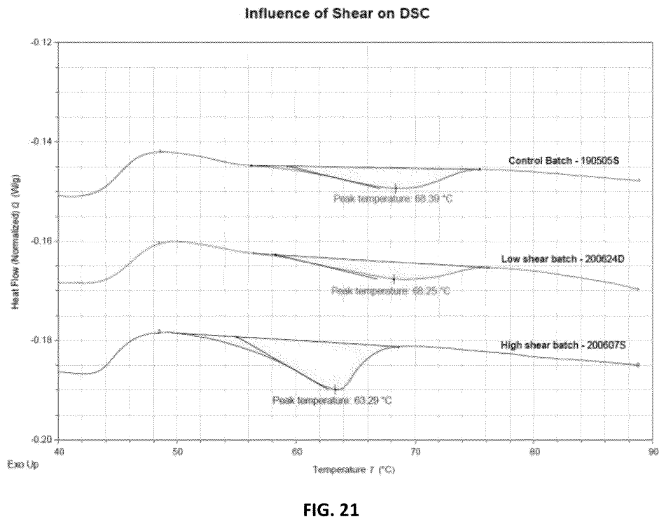

A batch was manufactured with high shear (rotor stator speed at 2000 rpm) turned on from the beginning of the holding step until the end of manufacturing process. This batch had a TM4 value of 63° C. (FIG. 21), and Tmh crystals were not visually observed. In contrast, a control batch manufactured without high shear (except for 10 minutes with a rotor stator speed at 2000 rpm after addition of minocycline), exhibited Tmh crystals and a TM4 value of 68° C. (FIG. 21). Accordingly, it appears that the use of high shear during the holding step of the manufacturing process can be detrimental to the formation of Tmh crystals and further that the prolonged use of high shear may break or eliminates part or all of the Tmh crystals or enable part all of them to form other crystals. On the other hand, following completion of the holding step the presence of high shear for a short period such as 10 mins during or after adding drug in order to facilitate rapid formation of a homogenous mixture did not appear to have a marked or detrimental effect on the Tmh crystals.

A batch was manufactured with low shear (rotor stator speed at 500 rpm) turned on from the beginning of the holding step until the end of manufacturing process. This batch exhibited TMH crystals and a TM4 value of 68° C. (FIG. 21). Accordingly, it appears the use of low shear during the holding step is not preventing the formation of TMH crystals although prolonged use of low shear might break or eliminate some Tmh crystals or enable some to form other crystals.

TABLE 57
Compositions and methods in FIG. 21

| Shear Study | Low shear | Control Batch | High Shear |
|---|---|---|---|
| | 500 rpm | | 2000 rpm |
| Formulation # | 200624D | 190505S | 200607S |
| HCO amount | 1.2% | 1.2% | 1.2% |
| API | MCH + ADP | MCH + ADP | MCH + ADP |
| Process | Holding process | Holding process | Holding process |
| | 54 C. for 4 hours | 54 C. for 4 hours | 54 C. for 4 hours |
| Component | | | |
| Soybean oil | 50 | 50 | 50 |
| coconut oil | 23.6 | 23.6 | 23.6 |
| Light mineral oil | 3.3 | 3.3 | 3.3 |
| Stearic acid | 3 | 3 | 3 |
| Docosanol | 1.1 | 1.1 | 1.1 |
| Hydrogenated castor oil | 1.2 | 1.2 | 1.2 |
| White wax (beeswax) | 2 | 2 | 2 |
| Stearyl alcohol | 1.5 | 1.5 | 1.5 |
| Cetostearyl alcohol | 3.5 | 3.5 | 3.5 |
| Myristyl alcohol | 2.5 | 2.5 | 2.5 |
| Cyclomethicone | 5 | 5 | 5 |
| Minocycline HCl | 3 | 3 | 3 |
| Adapalene | 0.3 | 0.3 | 0.3 |
| TOTAL | 100 | 100 | 100 |

### Part B

### Further Studies on the Effect of Shear

Using the same composition as control batch in Table 57, a batch is manufactured with a modified rotor-stator having a larger gap between rotor and stator. The larger gap enables a decrease in the shear applied to the product, while having the same rotational speed, compared to a smaller gap.

Using the same composition as control batch in Table 57, a batch is manufactured with an external pump connected to a loop, utilized to circulate the bulk of the batch from the bottom of the tank to the top of the tank. Utilizing of the external pump allows to achieve circulation without using a rotor-stator assembly at high speed. External pump can be a piston pump, a positive displacement pump, a peristaltic pump, or a pump of a different design which does not produce high shear.

Using the same composition as control batch in Table 57, a batch is manufactured with a rotor-stator providing high shear, but being turned on intermittently only. The rotor-stator can be turned on, for example, for 1 minute every 2 minutes, for 1 minute every 5 minutes, for 1 minute every 10 minutes, or for any other interval of time as will be appreciated by the skilled in the art.

Using the same composition as control batch in Table 57, a batch is manufactured utilizing high shear throughout the process with the exception of the holding step. During the holding step, either no high shear or low shear can be applied.

### Part C

Microscopic Examination after Continual Shear

Preliminary microscopic examination was made of a sample of a vehicle (without minocycline or adapalene) having the formulation indicated in Table 18A (placebo), prepared with 4 hours holding at about 54° C. using high shear mixing during the manufacturing process.

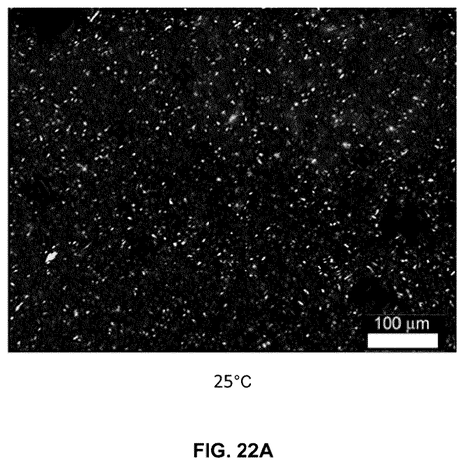

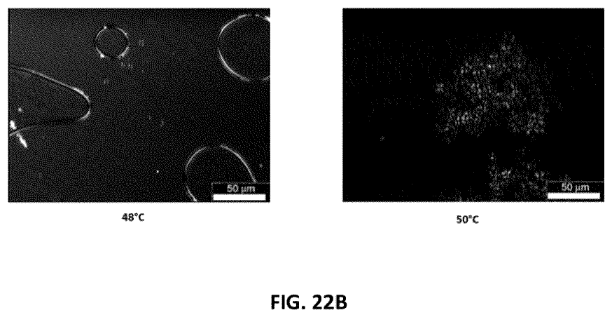

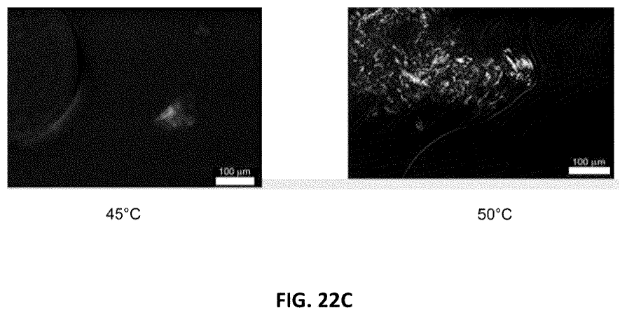

The microscopic examination at 25° C. indicated the presence of plates (FIG. 22A) At 25° C., the few Tmh and spherulites present tended to be masked by numerous plates. The microscopic examination at 45° C. indicated the presence of what seemed to be very few spherulites (FIG. 22B) and Tmh crystals (FIG. 22C). Plates were not visible (as they melted at that temperature). The spherulites and Tmh crystals were very small compared to a holding process without high shear during the manufacturing process, including the holding step.

Structures were also observed at 45° C. that did not have a defined form and may be pieces of what could have been either spherulites or Tmh crystals which were broken down due to the use of high shear mixing throughout the manufacturing process (FIG. 22C).

The sample prepared with high shear appears from a preliminary microscopic examination to have a unique crystal structure with plates and few Tmh crystals or spherulites plus structures that may be broken pieces of crystals.

### Example 34. Alternative Hydrogenated Oil Formulations

Formulations containing 1.2% of Alternative Hydrogenated Oils with 3% minocycline HCl, and 0.3% adapalene (MCH+ADP), are prepared in a holding process. Alternative Hydrogenated oils comprise Hydrogenated cottonseed oil and Hydrogenated soybean oil (Table 58).

TABLE 58
Compositions with Alternative Hydrogenated Oils

| | Hydrogenated | Hydrogenated |
|---|---|---|
| | cottonseed oil | soybean oil |
| HCO amount | 1.2% | 1.2% |
| API | MCH + ADP | MCH + ADP |
| Process | Holding process | Holding process |
| | 54° C. for 4 hours | 54° C. for 4 hours |
| Component | | |
| Soybean oil | 50 | 50 |
| coconut oil | 23.6 | 23.6 |
| Light mineral oil | 3.3 | 3.3 |
| Stearic acid | 3 | 3 |
| Docosanol | 1.1 | 1.1 |
| Hydrogenated | 1.2 | — |
| cottonseed oil | | |
| Hydrogenated | — | 1.2 |
| soybean oil | | |
| White wax (beeswax) | 2 | 2 |
| Stearyl alcohol | 1.5 | 1.5 |
| Cetostearyl alcohol | 3.5 | 3.5 |
| Myristyl alcohol | 2.5 | 2.5 |
| Cyclomethicone | 5 | 5 |
| Minocycline HCl | 3 | 3 |
| Adapalene | 0.3 | 0.3 |
| TOTAL | 100 | 100 |

### Exemplary Embodiments

1. A composition comprising: a tetracycline antibiotic and/or a retinoid, and a hydrogenated castor oil, wherein the composition comprises crystals having a fingerprint comprising one or more of:
a) Tmh crystals having a cross-sectional area of about 40-150 μm² on average (e.g., about 50-130 μm² on average, e.g., about 55-70 μm² on average, e.g., about 61-63 μm² on average, e.g. about 100-122 μm² on average);
b) a larger percentage by area of Tmh crystals as compared to the percentage of spherulite or plate crystals in the composition (e.g., at least about 25% Tmh crystals, e.g., at least about 30%, e.g., at least about 33%, e.g., about 30-33% Tmh crystals and less than about 20-23% spherulites and about 6-10% plates);
c) a larger percentage of Tmh crystals in a sample as compared to the percentage of spherulites in the composition (e.g., at least about 60% Tmh crystals, e.g., at least about 80%, e.g. at least about 90%, e.g. about 100% Tmh crystals);
d) a melting temperature above 50° C. as measured by differential scanning calorimetry (DSC), e.g., a melting temperature above 60° C., e.g., a melting temperature of about 50-80° C.;
e) a melting temperature higher than observed for a composition lacking Tmh crystals or a composition having a smaller percentage by area of Tmh crystals as compared to the percentage of spherulite or plate crystals in the composition;
f) a higher enthalpy (e.g., at least about 10% more, at least about 35% more, or at least about 100% more, or about 110% or more) than observed for a composition lacking Tmh crystals or a composition having a smaller percentage by area of Tmh crystals as compared to the percentage of spherulite or plate crystals in the composition;
g) a stronger hydrogen bond between crystals (e.g., a shift to a lower frequency and/or higher intensity waveband, as measured by FTIR) than observed for a composition lacking Tmh crystals or having a smaller percentage by area of Tmh crystals as compared to the percentage of spherulite or plate crystals in the composition;
h) Tmh crystals having stronger interactions between unit cells, wherein the unit cells are tangled fibers;
i) a crystal pattern substantially similar to a crystal pattern for a holding process shown in any subpart of FIG. 2; and/or
j) an average intensity by small angle X-ray scattering higher (e.g. at least 50% more, at least about 100% or at least about 200%) than that observed for a composition lacking Tmh crystals or a composition having a smaller percentage by area of Tmh crystals as compared to the percentage of spherulite or plate crystals in the composition; and or
k) a Raman spectra. with a peak in the range 1400-1500 cm⁻¹ having one or two shoulders, wherein the peak is at about 1446 cm⁻¹ and has a shoulder at about 1465 cm⁻¹ and/or has a shoulder at about 1425 cm⁻¹.
   2. The composition of embodiment 1, wherein the composition comprises Tmh crystals and plates, wherein the percentage of Tmh crystals exceeds the percentage of plates, as measured by area in a sample of the composition.
   3. The composition of embodiment 1, wherein the composition comprises Tmh crystals and spherulites, wherein the percentage of Tmh crystals exceeds the percentage of spherulites, as measured by area in a sample of the composition.
   4. The composition of embodiment 3, wherein the composition comprises at least 90% Tmh crystals, e.g. at least 95% Tmh crystals, e.g., at least 98% Tmh crystals, e.g. about 100% Tmh crystals, as measured by area in a sample of the composition.
   5. The composition of embodiment 1, wherein the composition comprises Tmh crystals and spherulites, wherein the number of Tmh crystals exceeds the number of spherulites, as counted in a sample of the composition.
   6. The composition of embodiment 5, wherein the composition comprises at least 85% Tmh crystals, e.g., at least 90% Tmh crystals, e.g. at least 95% Tmh crystals, e.g. 100% Tmh crystals, as measured by a count of crystals in a sample of the composition.
   7. The composition of embodiment 1, wherein the tetracycline antibiotic is one or more of tetracycline, oxytetracycline, demeclocycline, doxycycline hyclate, lymecycline, meclocycline, methacycline, minocycline, minocycline hydrochloride, rolitetracycline, chlorotetracycline, or tigecycline.
   8. The composition of any preceding embodiment, wherein the composition is a gel, ointment, liquid, semi-solid liquid, foamable composition, or foam.
   9. The composition of any preceding embodiment, wherein the composition is prepared by a process comprising:
a) formulating a mixture comprising at least one emollient, at least one foam adjuvant, and hydrogenated castor oil;
b) heating the mixture to a temperature sufficient to completely melt the at least one emollient, the at least one foam adjuvant, and the hydrogenated castor oil;
c) cooling the mixture to a temperature of about 48-60° C., e.g., about 48-56° C., e.g., about 50-58° C., e.g., about 48-54° C., e.g., 52-56° C., e.g., about 54° C., and maintaining the mixture at that temperature for about 1-2 or about 2-72 hours, e.g., about 2-16 hours, e.g., about 2-12 hours, e.g., about 2-6 hours, e.g., about 4 hours; and
d) cooling the mixture while mixing and optionally adding one or more agents.
   10. The composition of embodiment 9, wherein cooling the mixture of step (d) comprises one or more of:
a) cooling to a temperature of about 35° C. to about 40° C.;
b) cooling to a temperature of about 24° C. to about 28° C.; and/or
c) cooling to a temperature of about 22° C. to about 28° C.;
   11. The composition of any of embodiments 9 or 10, wherein the tetracycline antibiotic is added during step (d).
   12. The composition of any of embodiments 9 or 10, wherein cyclomethicone is added during step (d).
   13. The composition of any of embodiments 9 or 10, wherein the retinoid is added during step (d).
   14. The composition of any preceding embodiment, further comprising after step (d), stirring the mixture for up to 24 hours at a temperature of about 20C. to about 26° C.
   15. The composition of embodiment 9, wherein the formulation has increased stability over a composition prepared without step (c) after one or more weeks at 25 QC.
   16. The composition of embodiment 9, wherein the formulation has increased shakability and/or flowability over a composition prepared without step (c) after one or more weeks at 25° C.
   17. The composition of embodiment 9, wherein the formulation has stronger hydrogen bonds as compared to a composition prepared without step (c), e.g., as measured by FTIR.
   18. The composition of any preceding embodiment, wherein the tetracycline antibiotic is present in the composition at a concentration of about 0.5% to about 10% by weight, e.g., a concentration of about 1% to about 4% by weight, e.g., a concentration of about 1.5% by weight, e.g., a concentration of about 3% by weight.
   19. The composition of any preceding embodiment, wherein the tetracycline antibiotic is a minocycline and wherein the minocycline is present in the composition at a concentration of about 1.5% or about 3.0% by weight or between about 1.5% and about 3% by weight.
   20. The composition of embodiment 19, wherein the minocycline is minocycline hydrochloride.
   21. The composition of any preceding embodiment, wherein the composition comprises a tetracycline antibiotic and a retinoid.
   22. The composition of embodiment 21, wherein the retinoid is adapalene or tazarotene.
   23. The composition of embodiment 21, wherein the retinoid is adapalene.
   24. The composition of any preceding embodiment, wherein the retinoid is present in the composition at a concentration of about 0.1% to about 1% by weight, e.g., about 0.1% to about 0.5% by weight.
   25. The composition of any preceding embodiment, wherein the retinoid is present in the composition at a concentration of about 0.2% by weight.
   26. The composition of any preceding embodiment, wherein the retinoid is present in the composition at a concentration of about 0.3% by weight.
   27. The composition of any preceding embodiment, wherein the retinoid is present in the composition at a concentration of about 0.4% by weight.
   28. The composition of any preceding embodiment, further comprising a wax that is solid at about 36° C. or at about 49° C..
   29. The composition of any preceding embodiment, wherein the retinoid is adapalene and wherein the adapalene is present in the composition at a concentration of about 0.1% or about 0.5% by weight or between about 0.1% about 0.3% by weight.
   30. The composition of any preceding embodiment, wherein the composition comprises about 1-3%, about 1-2%, or about 1.2%, hydrogenated castor oil.
   31. The composition of any preceding embodiment, wherein the composition is a foam composition.
   32. The composition of embodiment 31, wherein the composition further comprises a propellant.
   33. The composition of embodiments 31 or 32, further comprises one or more foam adjuvants and/or a wax capable of forming at foam.
   34. The composition of any one of embodiments 1-33, wherein the composition further comprises (a) about 60% to about 95% by weight of at least one emollient and (b) about 5% to about 25% by weight at least one foam adjuvant, or a combination thereof.
   35. The composition of any one of embodiments 1-34, wherein the composition comprises: a) about 40% to about 60% by weight of soybean oil; b) about 20% to about 25% by weight of coconut oil; c) about 2% to about 8% by weight of light mineral oil; d) about 2% to about 4% by weight of stearic acid; e) about 0.6% to about 1.6% by weight of docosanol; f) about 1% to about 2% by weight of hydrogenated castor oil; g) about 1% to about 3% by weight of white wax (such as beeswax); h) about 1% to about 2% by weight of stearyl alcohol; i) about 2.0% to about 5.0% by weight of cetostearyl alcohol; j) about 1.8% to about 3.3% by weight of myristyl alcohol; k) about 3.0% to about 7.0% by weight of cyclomethicone; I) about 1% to about 5% (such as 1%, 1.5%, 2%, 2.5%, 3%, 3.5%, 4%, 4.5%, or 5%) by weight of minocycline hydrochloride and m) about 0.1% to about 0.5% (such as 0.1%, 0.2%, 0.3%, 0.4%, or 0.5%) by weight of adapalene.
   36. The composition of any one of embodiments 1-35, wherein the composition comprises: a) about 40% to about 60% by weight of soybean oil; b) about 20% to about 25% by weight of coconut oil; c) about 2% to about 8% by weight of light mineral oil; d) about 2% to about 4% by weight of stearic acid; e) about 0.6% to about 1.6% by weight of docosanol; f) about 0.1% to about 2% by weight of hydrogenated castor oil; g) about 0.1% to about 3% by weight of a white wax (such as beeswax); h) about 1% to about 2% by weight of stearyl alcohol; i) about 2.0% to about 5.0% by weight of cetostearyl alcohol; j) about 1.8% to about 3.3% by weight of myristyl alcohol; k) about 3.0% to about 7.0% by weight of cyclomethicone; I) about 1% to about 5% (such as 1%, 1.5%, 2%, 2.5%, 3%, 3.5%, 4%, 4.5%, or 5%) by weight of minocycline hydrochloride and m) about 0.1% to about 0.5% (such as 0.1%, 0.2%, 0.3%, 0.4%, or 0.5%) by weight of adapalene.
   37. The composition of any one of embodiments 1-36, wherein the composition comprises:
i) about 50% by weight of soybean oil;
ii) about 23.6% by weight of coconut oil;
iii) about 3.3% to about 6.6% by weight of light mineral oil;
iv) about 3% by weight of stearic acid;
v) about 1.1% by weight of docosanol;
vi) about 1.2% by weight of hydrogenated castor oil;
vii) about 3.5% by weight of cetostearyl alcohol;
viii) about 2% by weight of white wax (such as beeswax);
ix) about 1.5% by weight of stearyl alcohol;
x) about 2.5% by weight of myristyl alcohol;
xi) about 5% by weight of cyclomethicone;
xii) about 3% by weight of minocycline; and
xiii) about 0.3% by weight of adapalene.
   38. The composition of embodiment 37, wherein the minocycline is minocycline hydrochloride.
   39. The composition of embodiment 34, wherein the composition has a phase transition temperature TM4 of about 50-80° C., e.g., 68-69° C. or about 68-72° C., as measured by DSC.
   40. The composition of embodiment 34, wherein the composition has a wavenumber band of about 3301-3312 cm⁻¹ when stored at 25° C., or about 3320-3324 cm⁻¹ when stored at 50° C. as measured by FTIR.
   41. The composition of any preceding embodiment, wherein the composition is characterized by showing an average intensity by small angle X-ray scattering higher than that observed for a composition having a lower percentage by area of Tmh crystals as compared to the percentage of spherulite or plate crystals in the composition.
   42. The composition of embodiment 41, wherein the composition shows an average intensity by small angle X-ray scattering at least about 50% more, at least about 100% more, or at least about 200% more than that observed for a composition having a lower percentage by area of Tmh crystals than the percentage of spherulite or plate crystals in the composition.
   43. A composition comprising: a tetracycline antibiotic and/or a retinoid and/or hydrogenated castor oil, wherein the foam composition comprises crystals, wherein the percentage of Tmh crystals, e.g., crystals comprising nonuniform shapes, exceeds the percentage of other types of crystals, such as microcrystals, needles, plates, rods, or a combination thereof.
   44. The composition of embodiment 43, wherein the composition comprises about 1-3%, e.g., about 1-2%, e.g., about 1.2%, of hydrogenated castor oil.
   45. The composition of embodiments 43 or 44, further comprising at least one emollient and at least one foam adjuvant.
   46. The composition of any one of embodiments 43-45, prepared by a process comprising:
a) formulating a mixture comprising the at least one emollient, the at least one foam adjuvant, and the hydrogenated castor oil;
b) heating the mixture to a temperature sufficient to completely melt the at least one emollient, the at least one foam adjuvant, and the hydrogenated castor oil;
c) cooling the mixture to a temperature of about 50-60° C.. e.g., about 54° C., and maintaining the mixture at the temperature for about 1-72 hours, e.g., about 1-12 or 2-16 hours, e.g., about 2-6 hours, e.g., about 4 hours;
d) cooling the mixture while mixing and optionally adding one or more additional agents.
   47. The composition of embodiment 46, wherein cooling the mixture of step (d) comprises one or more of:
a) cooling to a temperature of about 35° C. to about 40° C.;
b) cooling to a temperature of about 24° C. to about 28° C.; and/or
c) cooling to a temperature of about 22° C. to about 28° C.;
   48. The composition of any of embodiments 46 or 47, wherein the tetracycline antibiotic is added during step (d).
   49. The composition of any of embodiments 46 or 47, wherein cyclomethicone is added during step (d).
   50. The composition of any of embodiments 46 or 47, wherein the retinoid is added during step (d).
   51. The composition of any one of embodiments 46-50, further comprising after step (d), stirring the mixture for up to 24 hours at a temperature of about 20° C. to about 26° C..
   52. The composition of any one of embodiments 1-51, wherein the composition is packaged in an aerosol container and pressurized with a propellant, and the composition produces a foam upon release from the container.
   53. A foam produced from the composition released from the aerosol container of embodiment 52.
   54. The composition or foam of any one of embodiments 1-53, wherein the crystals in the foam or composition comprises Tmh crystals.
   55. The composition or foam of any one of embodiments 1-54, wherein a majority of the crystals in the foam or composition by area comprises Tmh crystals.
   56. The foam or composition of any one of embodiments 1-55, wherein the foam or composition is lacking spherulites and comprises by area less than a majority of plates.
   57. The foam or composition of any one of embodiments 1-56, wherein the hydrogenated castor oil forms Tmh crystals in the foam composition.
   58. The foam or composition of any one of embodiments 1-57, wherein the hydrogenated castor oil forms plates in the foam composition.
   59. The foam or composition of any one of embodiments 1-58, wherein the hydrogenated castor oil does not form spherulites in the foam composition.
   60. The composition of any one of embodiments 1-59, wherein the composition has an improved shakability compared to a composition prepared without a hold step (e.g., without step (c) of embodiment 10).
   61. The composition of embodiment 60, wherein the composition is shakable for at least 1 day at 5° C.
   62. The composition of embodiment 60, wherein the composition is shakable for at least 14 days at 5° C.
   63. The composition of embodiment 60, wherein the composition is shakable for at least 15 days at 5° C.
   64. The composition of embodiment 60, wherein the composition is shakable for at least 30 days at 5° C.
   65. The composition of embodiment 60, wherein the composition is shakable for at least 60 days at 5° C.
   66. The composition of embodiment 60, wherein the composition is shakable for at least 90 days at 5° C.
   67. The composition of embodiment 60, wherein the composition is shakable for at least 180 days at 5° C.
   68. The composition of embodiment 60, wherein the composition is shakable for at least 1 day at 25° C.
   69. The composition of embodiment 60, wherein the composition is shakable for at least 14 days at 25° C.
   70. The composition of embodiment 60, wherein the composition is shakable for at least 15 days at 25° C.
   71. The composition of embodiment 60, wherein the composition is shakable for at least 30 days at 25° C.
   72. The composition of embodiment 60, wherein the composition is shakable for at least 60 days at 25° C.
   73. The composition of embodiment 60, wherein the composition is shakable for at least 90 days at 25° C.
   74. The composition of embodiment 60, wherein at 25° C. the composition is shakable for at least 180 days, or for at least 12 months, or for at least 18 months, or for at least 24 months.
   75. The composition of embodiment 60, wherein the composition is shakable for at least 1 day at 40° C.
   76. The composition of embodiment 60, wherein the composition is shakable for at least 14 days at 40° C.
   77. The composition of embodiment 60, wherein the composition is shakable for at least 15 days at 40° C.
   78. The composition of embodiment 60, wherein the composition is shakable for at least 30 days at 40° C.
   79. The composition of embodiment 60, wherein the composition is shakable for at least 60 days at 40° C.
   80. The composition of embodiment 60, wherein the composition is shakable for at least 90 days at 40° C.
   81. The composition of embodiment 60, wherein the composition is shakable for at least 180 days at 40° C.
   82. The composition of any one of embodiments 1-81, wherein the composition does not block a foam valve of the aerosol container for at least 1 day.
   83. The composition of embodiment 82, wherein the composition does not block a foam valve of the aerosol container for at least 10 days.
   84. The composition of embodiment 82, wherein the composition does not block a foam valve of the aerosol container for at least 15 days.
   85. The composition of embodiment 82, wherein the composition does not block a foam valve of the aerosol container for at least 30 days.
   86. The composition of embodiment 82, wherein the composition does not block a foam valve of the aerosol container for at least 60 days.
   87. The composition of embodiment 82, wherein the composition does not block a foam valve of the aerosol container for at least 90 days.
   88. The composition of embodiment 82, wherein the composition does not block a foam valve of the aerosol container for at least 180 days.
   89. The composition of any one of embodiments 1-88, wherein the composition has a G′ value between about 20 Pa and about 50 Pa after 1 day at 25° C.
   90. The composition of any one of embodiments 1-88, wherein the composition has a G′ value between about 50 Pa and about 100 Pa after 1 day at 25° C.
   91. The composition of embodiment 89, wherein the composition has a G′ value between about 100 Pa and about 200 Pa after 1 day at 25° C.
   92. The composition of embodiment 89, wherein the composition has a G′ value between about 200 Pa and about 300 Pa after 1 day at 25° C.
   93. This composition of embodiment 89, wherein the composition has a G′ value between about 300 Pa and about 400 Pa after 1 day at 25° C.
   94. The composition of embodiment 89, wherein the composition has a G′ value between about 100 Pa and about 150 Pa after 15 days at 25° C.
   95. The composition of embodiment 89, wherein the composition has a G′ value between about 100 Pa and about 200 Pa after 15 days at 25° C.
   96. The composition of embodiment 89, wherein the composition has a G′ value between about 150 Pa and about 200 Pa after 15 days at 25° C.
   97. The composition of embodiment 89, wherein the composition has a G′ value between about 200 Pa and about 300 Pa after 15 days at 25° C.
   98. The composition of embodiment 89, wherein the composition has a G′ value between about 200 Pa and about 250 Pa after 15 days at 25° C.
   99. The composition of embodiment 89, wherein the composition has a G′ value between about 250 Pa and about 300 Pa after 15 days at 25° C.
   100. The composition of embodiment 89, wherein the composition has a G′ value between about 300 Pa and about 400 Pa after 15 days at 25° C.
   101. The composition of embodiment 89, wherein the composition has a G′ value between about 50 Pa and about 100 Pa after 30 days at 25° C.
   102. The composition of embodiment 89, wherein the composition has a G′ value between about 100 Pa and about 200 Pa after 30 days at 25° C.
   103. The composition of embodiment 89, wherein the composition has a G′ value between about 200 Pa and about 300 Pa after 30 days at 25° C.
   104. The composition of embodiment 89, wherein the composition has a G′ value between about 300 Pa and about 400 Pa after 30 days at 25° C.
   105. The composition of embodiment 89, wherein the composition has a G′ value between about 400 Pa and about 500 Pa after 30 days at 25° C.
   106. The composition of embodiment 89, wherein the composition has a G′ value between about 500 Pa and about 600 Pa after 30 days at 25° C.
   107. The composition of embodiment 89, wherein the composition has a G′ value between about 500 Pa and about 550 Pa after 30 days at 25° C.
   108. The composition of embodiment 89, wherein the composition has a G′ value between about 550 Pa and about 600 Pa after 30 days at 25° C.
   109. The composition of embodiment 89, wherein the composition has a G′ value between about 2000 Pa and about 4000 Pa after 1 day at 40° C.
   110. The composition of embodiment 89, wherein the composition has a G′ value between about 4000 Pa and about 5000 Pa after 1 day at 40° C.
   111. The composition of embodiment 89, wherein the composition has a G′ value between about 4000 Pa and about 4500 Pa after 1 day at 40° C.
   112. The composition of embodiment 89, wherein the composition has a G′ value between about 4500 Pa and about 5000 Pa after 1 day at 40° C.
   113. The composition of embodiment 89, wherein the composition has a G′ value between about 5000 Pa and about 6000 Pa after 1 day at 40° C.
   114. The composition of embodiment 89, wherein the composition has a G′ value between about 6000 Pa and about 7000 Pa after 1 day at 40° C.
   115. The composition of embodiment 89, wherein the composition has a G′ value between about 7000 Pa and about 8000 Pa after 1 day at 40° C.
   116. The composition of embodiment 89, wherein the composition has a G′ value between about 8000 Pa and about 9000 Pa after 1 day at 40° C.
   117. The composition of embodiment 89, wherein the composition has a G′ value between about 9000 Pa and about 10000 Pa after 1 day at 40° C.
   118. The composition of embodiment 89, wherein the composition has a G′ value between about 10000 Pa and about 11000 Pa after 1 day at 40° C.
   119. The composition of embodiment 89, wherein the composition has a G′ value between about 11000 Pa and about 12000 Pa after 1 day at 40° C.
   120. The composition of embodiment 89, wherein the composition has a G′ value between about 12000 Pa and about 13000 Pa after 1 day at 40° C.
   121. The composition of embodiment 89, wherein the composition has a G′ value between about 13000 Pa and about 14000 Pa after 1 day at 40° C.
   122. The composition of embodiment 89, wherein the composition has a G′ value between about 14000 Pa and about 15000 Pa after 1 day at 40° C.
   123. The composition of embodiment 89, wherein the composition has a G′ value between about 15000 Pa and about 16000 Pa after 1 day at 40° C.
   124. The composition of embodiment 89, wherein the composition has a G′ value between about 16000 Pa and about 17000 Pa after 1 day at 40° C.
   125. The composition of embodiment 89, wherein the composition has a G′ value between about 2000 Pa and about 5000 Pa after 15 days at 40° C.
   126. The composition of embodiment 89, wherein the composition has a G′ value between about 5000 Pa and about 6000 Pa after 15 days at 40° C.
   127. The composition of embodiment 89, wherein the composition has a G′ value between about 6000 Pa and about 7000 Pa after 15 days at 40° C.
   128. The composition of embodiment 89, wherein the composition has a G′ value between about 7000 Pa and about 8000 Pa after 15 days at 40° C.
   129. The composition of embodiment 89, wherein the composition has a G′ value between about 8000 Pa and about 9000 Pa after 15 days at 40° C.
   130. The composition of embodiment 89, wherein the composition has a G′ value between about 9000 Pa and about 10000 Pa after 15 days at 40° C.
   131. The composition of embodiment 89, wherein the composition has a G′ value between about 10000 Pa and about 11000 Pa after 15 days at 40° C.
   132. The composition of embodiment 89, wherein the composition has a G′ value between about 11000 Pa and about 12000 Pa after 15 days at 40° C.
   133. The composition of embodiment 89, wherein the composition has a G′ value between about 12000 Pa and about 13000 Pa after 15 days at 40° C.
   134. The composition of embodiment 89, wherein the composition has a G′ value between about 13000 Pa and about 14000 Pa after 15 days at 40° C.
   135. The composition of embodiment 89, wherein the composition has a G′ value between about 14000 Pa and about 15000 Pa after 15 days at 40° C.
   136. The composition of embodiment 89, wherein the composition has a G′ value between about 15000 Pa and about 16000 Pa after 15 days at 40° C.
   137. The composition of embodiment 89, wherein the composition has a G′ value between about 16000 Pa and about 17000 Pa after 15 days at 40° C.
   138. The composition of embodiment 89, wherein the composition has a G′ value between about 17000 Pa and about 18000 Pa after 15 days at 40° C.
   139. The composition of embodiment 89, wherein the composition has a G′ value between about 18000 Pa and about 19000 Pa after 15 days at 40° C.
   140. The composition of embodiment 89, wherein the composition has a G′ value between about 2000 Pa and about 5000 Pa after 30 days at 40° C.
   141. The composition of embodiment 89, wherein the composition has a G′ value between about 5000 Pa and about 6000 Pa after 30 days at 40° C.
   142. The composition of embodiment 89, wherein the composition has a G′ value between about 6000 Pa and about 7000 Pa after 30 days at 40° C.
   143. The composition of embodiment 89, wherein the composition has a G′ value between about 7000 Pa and about 8000 Pa after 30 days at 40° C.
   144. The composition of embodiment 89, wherein the composition has a G′ value between about 8000 Pa and about 9000 Pa after 30 days at 40° C.
   145. The composition of embodiment 89, wherein the composition has a G′ value between about 9000 Pa and about 10000 Pa after 30 days at 40° C.
   146. The composition of embodiment 89, wherein the composition has a G′ value between about 10000 Pa and about 11000 Pa after 30 days at 40° C.
   147. The composition of embodiment 89, wherein the composition has a G′ value between about 11000 Pa and about 12000 Pa after 30 days at 40° C.
   148. The composition of embodiment 89, wherein the composition has a G′ value between about 12000 Pa and about 13000 Pa after 30 days at 40° C.
   149. The composition of embodiment 89, wherein the composition has a G′ value between about 13000 Pa and about 14000 Pa after 30 days at 40° C.
   150. The composition of embodiment 89, wherein the composition has a G′ value between about 14000 Pa and about 15000 Pa after 30 days at 40° C.
   151. The composition of embodiment 89, wherein the composition has a G′ value between about 15000 Pa and about 16000 Pa after 30 days at 40° C.
   152. The composition of embodiment 89, wherein the composition has a G′ value between about 16000 Pa and about 17000 Pa after 30 days at 40° C.
   153. The composition of embodiment 89, wherein the composition has a G′ value between about 17000 Pa and about 18000 Pa after 30 days at 40° C.
   154. The composition of embodiment 89, wherein the composition has a G′ value between about 18000 Pa and about 19000 Pa after 30 days at 40° C.
   155. The foam or composition of any one of embodiments 1-154, wherein the foam or composition is capable of softening sebum.
   156. The foam or composition of embodiment 155, wherein the melting temperature of a 1:1 mixture of sebum and the foam or composition is 10% lower than the melting temperature of sebum.
   157. The foam or composition of embodiment 155, wherein the melting temperature of a 1:1 mixture of sebum and the foam or composition is 20% lower than the melting temperature of sebum.
   158. The foam or composition of embodiment 155, wherein the melting temperature of a 1:1 mixture of sebum and the foam or composition is 30% lower than the melting temperature of sebum.
   159. The foam or composition of embodiment 155, wherein the melting temperature of a 1:1 mixture of sebum and the foam or composition is 40% lower than the melting temperature of sebum.
   160. The foam or composition of embodiment 155, wherein the melting temperature of a 1:1 mixture of sebum and the foam or composition is 50% lower than the melting temperature of sebum.
   161. The foam or composition of embodiment 155, wherein the melting temperature of a 1:1 mixture of sebum and the foam or composition is 60% lower than the melting temperature of sebum.
   162. The foam or composition of embodiment 155, wherein the melting temperature of a 1:1 mixture of sebum the foam or composition is 70% lower than the melting temperature of sebum.
   163. The foam or composition of embodiment 155, wherein the melting temperature of a 1:1 mixture of sebum and the foam or composition is 80% lower than the melting temperature of sebum.
   164. The foam or composition of embodiment 155, wherein the melting temperature of a 1:1 mixture of sebum and the foam or composition is 90% lower than the melting temperature of sebum.
   165. The foam or composition of embodiment 155, wherein the melting temperature of a 1:1 mixture of sebum and the foam or composition is 100% lower than the melting temperature of sebum.
   166. The foam or composition of any one of embodiments 155-165, wherein the melting point of the 1:1 mixture of sebum and the foam or composition is between about 30.0° C. and 31.0° C.
   167. The foam or composition of any one of embodiments 155-165, wherein the melting point of the 1:1 mixture of sebum and the foam or composition is between about 31.0° C. and 32.0° C.
   168. The foam or composition of any one of embodiments 155-165, wherein the melting point of the 1:1 mixture of sebum and the foam or composition is between about 32.0° C. and 33.0° C.
   169. The foam or composition of any one of embodiments 155-165, wherein the melting point of the 1:1 mixture of sebum and the foam or composition is between about 33.0° C. and 34.0° C.
   170. The foam or composition of any one of embodiments 155-165, wherein the melting point of the 1:1 mixture of sebum and the foam or composition is between about 34.0° C. and 35.0° C.
   171. The foam or composition of any one of embodiments 155-165, wherein the melting point of the 1:1 mixture of sebum and foam formulation is between about 35.0° C. and 36.0° C.
   172. The foam or composition of any one of embodiments 155-165, wherein the melting point of the 1:1 mixture of sebum and the foam or composition is between about 36.0° C. and 37.0° C.
   173. The foam or composition of any one of embodiments 155-165, wherein the melting point of the 1:1 mixture of sebum and the foam or composition is between about 37.0° C. and 38.0° C.
   174. The foam or composition of any one of embodiments 155-165, wherein the melting point of the 1:1 mixture of sebum and the foam or composition is between about 38.0° C. and 39.0° C.
   175. The foam or composition of any one of embodiments 155-165, wherein the melting point of the 1:1 mixture of sebum and the foam or composition is between about 39.0° C. and 40.0° C.
   176. The foam or composition of any one of embodiments 155-165, wherein the melting point of the 1:1 mixture of sebum and the foam or composition is between about 40.0° C. and 41.0° C.
   177. The foam or composition of any one of embodiments 155-165, wherein the melting point of the 1:1 mixture of sebum and the foam or composition is between about 41.0° C. and 42.0° C.
   178. The foam or composition of any one of embodiments 155-165, wherein the melting point of the 1:1 mixture of sebum and the foam or composition is between about 42.0° C. and 43.0° C.
   179. The composition of embodiment 54, wherein the crystals in the composition comprises Tmh crystals at a greater degree as compared to a comparable composition produced by a process that does not comprise a step of cooling the mixture to a temperature of about 50-60° C. and maintaining the mixture at the temperature for about 1-72, e.g., about 1-16, e.g., 1-2 or 2-16 hours.
   180. A composition comprising at least one tetracycline antibiotic, at least one retinoid, at least one emollient, at least one foam adjuvant, and hydrogenated castor oil, prepared by a process comprising:
a) formulating a mixture comprising the at least one emollient, the at least one foam adjuvant, the hydrogenated castor oil;
b) heating the mixture to a temperature sufficient to completely melt the at least one emollient, the at least one foam adjuvant, the hydrogenated castor oil;
c) cooling the mixture to a temperature of about 50-60° C., e.g., about 54° C., and maintaining the mixture at the temperature for about 1-72, e.g., about 1-16, e.g., 1-2 or 2-16 hours;
d) cooling the mixture while mixing and optionally adding one or more additional agents.
   181. The composition of embodiment 180, wherein cooling the mixture of step (d) comprises one or more of:
a) cooling to a temperature of about 35° C. to about 40° C.;
b) cooling to a temperature of about 24° C. to about 28° C.; and/or
c) cooling to a temperature of about 22° C. to about 28° C.;
   182. The composition of any of embodiments 180 or 181, wherein the tetracycline antibiotic is added during step (d).
   183. The composition of any of embodiments 180 or 181, wherein cyclomethicone is added during step (d).
   184. The composition of any of embodiments 180 or 181, wherein the retinoid is added during step (d).
   185. The composition of any one of embodiments 180-184, further comprising after step (d), stirring the mixture for up to 24 hours at a temperature of about 20° C. to about 26° C..
   186. The composition of any one of embodiments 180-185, wherein when the composition is packaged in an aerosol container and pressurized with a propellant, the composition produces a foam upon release from the container.
   187. The composition of any one of embodiments 180-186, wherein the composition comprises crystals.
   188. The composition of any one of embodiments 180-187, wherein a majority by area of the crystals comprise Tmh crystals, e.g., those having a nonuniform shape.
   189. The composition of embodiment 188, wherein a minority by area of the crystals comprises microcrystals, needles, plates, rods, or a combination thereof.
   190. The composition of embodiments 188 or 189, wherein the composition comprises plates.
   191. The composition of embodiments 188 or 189, wherein the composition lacks spherulites and comprises by area less than a majority of plates.
   192. The composition of embodiments 188-191, wherein the crystals in the composition comprises Tmh crystals at a greater degree as compared to a comparable composition produced by a process that does not comprise a step of cooling the mixture to a temperature of about 50-60° C. and maintaining the mixture at the temperature for about 1-72, e.g., about 1-16, e.g., 1-2 or 2-16 hours.
   193. The composition of any one of embodiments 188-192, wherein the composition comprises (a) about 60% to about 95% by weight of at least one emollient and (b) about 5% to about 25% by weight at least one foam adjuvant, or a combination thereof.
   194. The composition of any one of embodiments 188-193, wherein the composition comprises about 40% to about 60% by weight of soybean oil.
   195. The composition of any one of embodiments 188-194, wherein the composition comprises about 20% to about 25% by weight of coconut oil.
   196. The composition of any one of embodiments 188-195, wherein the composition comprises about 2% to about 8% by weight of light mineral oil.
   197. The composition of any one of embodiments 188-196, wherein the composition comprises about 3.5% to about 6.5% by weight of cyclomethicone.
   198. The composition of any one of embodiments 188-197, wherein the composition comprises about 2% to about 4% by weight of stearic acid.
   199. The composition of any one of embodiments 188-198, wherein the composition comprises about 0.6% to about 1.6% by weight of docosanol.
   200. The composition of any one of embodiments 188-199, wherein the composition comprises about 1% to about 2% by weight of hydrogenated castor oil.
   201. The composition of any one of embodiments 188-200, wherein the composition comprises about 1% to about 3% by weight of white wax (e.g., beeswax).
   202. The composition of any one of embodiments 188-201, wherein the composition comprises about 1% to about 2% by weight of stearyl alcohol.
   203. The composition of any one of embodiments 188-202, wherein the composition comprises about 2.0% to about 5.0% by weight of cetostearyl alcohol.
   204. The composition of any one of embodiments 188-203, wherein the composition comprises about 1.8% to about 3.3% by weight of myristyl alcohol.
   205. The composition of any one of embodiments 188-204, wherein the composition comprises about 3.0% to about 7.0% by weight of cyclomethicone.
   206. The composition of any one of embodiments 188-205, wherein the composition comprises about 1% to about 5% (such as 1%, 1.5%, 2%, 2.5%, 3%, 3.5%, 4%, 4.5%, or 5%) by weight of minocycline hydrochloride.
   207. The composition of any one of embodiments 188-206, wherein the composition comprises about 0.1% to about 0.5% (such as 0.1%, 0.2%, 0.3%, 0.4%, or 0.5%) by weight of adapalene.
   208. The composition of any one of embodiments 1-207, wherein the composition further comprises a surfactant.
   209. The composition of any one of embodiments 1-207, wherein the composition comprises less than about 10% of a surfactant.
   210. The composition of any one of embodiments 1-207, wherein the composition comprises less than about 5% of a surfactant.
   211. The composition of any one of embodiments 1-207, wherein the composition comprises less than about 3% of a surfactant.
   212. The composition of any one of embodiments 1-207, wherein the composition comprises less than about 2% of a surfactant.
   213. The composition of any one of embodiments 1-207, wherein the composition comprises less than about 1% of a surfactant.
   214. The composition of any one of embodiments 1-207, wherein the composition comprises less than about 0.1% of a surfactant.
   215. The composition of any one of embodiments 1-207, wherein the composition is free of water and/or surfactant.
   216. The composition of any one of embodiments 1-207, wherein the composition comprises less than about 10% of water.
   217. The composition of any one of embodiments 1-207, wherein the composition comprises less than about 5% of water.
   218. The composition of any one of embodiments 1-207, wherein the composition comprises less than about 3% of water.
   219. The composition of any one of embodiments 1-207, wherein the composition comprises less than about 2% of water.
   220. The composition of any one of embodiments 1-207, wherein the composition comprises less than about 1% of water.
   221. The composition of any one of embodiments 1-207, wherein the composition comprises less than about 0.1% of water.
   222. The composition of any one of embodiments 1-221 for use in a method of treating acne.
   223. The composition of embodiment 222, wherein the acne is acne vulgaris and/or acne conglobata.
   224. The composition of embodiment 223, wherein the acne vulgaris is a mild acne vulgaris.
   225. The composition of embodiment 223, wherein the acne vulgaris is a moderate acne vulgaris.
   226. The composition of embodiment 223, wherein the acne vulgaris is a severe acne vulgaris.
   227. The composition of embodiment 223, wherein the severity of vulgaris and/or acne conglobata, based on investigator's global assessment scale, ranges from 1 to 4.
   228. The composition of embodiment 223, wherein the severity of vulgaris and/or acne conglobata, based on investigator's global assessment scale, ranges from 3 to 4.
   229. The composition of embodiment 223, wherein the acne vulgaris and/or acne conglobata is characterized by inflammatory lesions.
   230. The composition of embodiment 223 wherein the acne vulgaris and/or acne conglobata is characterized by non-inflammatory lesions.
   231. The composition of embodiment 223, wherein the acne vulgaris and/or acne conglobata is characterized by papules and pustules.
   232. The composition of embodiment 231, wherein the papules and pustules are present in nose of the subject.
   233. The composition of embodiment 231, wherein the papules and pustules are present in cheek of the subject.
   234. The composition of embodiment 231, wherein the papules and pustules are present in chin of the subject.
   235. The composition of embodiment 231, wherein the papules and pustules are present in forehead of the subject.
   236. Use of the composition of any one of embodiments 1-235 in the manufacture of a medicament for treating acne.
   237. A method of treating a subject suffering from acne comprising administering the composition of any one of embodiments 1-236.
   238. A method of ameliorating, treating or preventing one or more symptoms associated with acne and/or rosacea in a subject, comprising administering the composition of any one of embodiments 1-237.
   239. The method of embodiment 238, wherein the one or more symptoms include telangiectasia, rhinophyma, and/or ocular rosacea.
   240. A method of reducing a risk of infection associated with acne or rosacea, comprising administering the composition of any preceding embodiment to a subject in need thereof.
   241. A method of treating a subject for acne or rosacea, comprising diagnosing the subject with acne or rosacea and administering the composition of any preceding embodiment to the subject diagnosed with acne or rosacea.
   242. A method of treating a subject for acne or rosacea, comprising diagnosing a subject having acne or rosacea as at risk for tissue damage and administering the composition of any preceding embodiment to the subject at risk for tissue damage.
   243. The method of embodiment 242, wherein the composition comprises: a) about 40% to about 60% by weight of soybean oil; b) about 20% to about 25% by weight of coconut oil; c) about 2% to about 8%/by weight of light mineral oil; d) about 3.5% to about 6.5% by weight of cyclomethicone; d) about 2% to about 4% by weight of stearic acid; e) about 0.6% to about 1.6% by weight of docosanol; f) about 1.2% hydrogenated castor oil; g) about 1% to about 3% by weight of beeswax; h) about 1% to about 2% by weight of stearyl alcohol; i) about 2.0% to about 5.0% by weight of cetostearyl alcohol; j) about 1.8% to about 3.3% by weight of myristyl alcohol; k) about 3.0% to about 7.0% by weight of cyclomethicone; I) about 1% to about 5% (such as 1%, 1.5%, 2%, 2.5%, 3%, 3.5%, 4%, 4.5%, or 5%) by weight of minocycline hydrochloride and m) about 0.1% to about 0.5% (such as 0.1%, 0.2%, 0.3%, 0.4%, or 0.5%) by weight of adapalene.
   244. A method of improving wax crystal stability, comprising:
a) formulating a mixture comprising at least one emollient, at least one foam adjuvant, and a hydrogenated castor oil;
b) heating the mixture to a first temperature sufficient to completely melt the at least one emollient, the at least one foam adjuvant, and the hydrogenated castor oil;
c) cooling the mixture; and
d) interposing one or more disruptive steps during the cooling;
   wherein the one or more disruptive steps comprises maintaining the composition for a holding period at one or more second temperatures that are below the first temperature; and wherein the second temperature is above about 37° C.
   245. The method of embodiment 244, wherein the holding period is about 2-8 hours, e.g., about 3-6 hours, e.g., about 4-5 hours.
   246. The method of embodiment 244, wherein the first temperature is about 10-90° C. higher than the second temperature.
   247. The method of embodiment 244 or 245, wherein the one or more second temperature is in a range from about 45-70° C., e.g. above 48° C., e.g., about 54° C.
   248. The method of embodiment 244, wherein the holding at the second temperature is for at least about 20 minutes to about 12 hours, e.g., about 2 hours, about 4 hours, about 6 hours, about 8 hours, about 10 hours, about 12 hours.
   249. A method of preparing a composition comprising at least one emollient and at least one wax, the method comprising;
a) mixing the at least one emollient and the at least one wax, and heating the mixture to a temperature sufficient to completely melt the mixture; and
b) cooling the mixture to a temperature of about 54° C., and holding at that temperature for about 4 hours;
c) cooling the mixture while mixing and optionally adding one or more agents.
   250. The method of embodiment 249, wherein the wax further comprises hydrogenated castor oil.
   251. A method of preparing a foamable composition comprising at least one emollient, at least one foam adjuvant, and hydrogenated castor oil, the method comprising;
a) mixing the at least one emollient, the at least one foam adjuvant, and hydrogenated castor oil, and heating the mixture to a temperature sufficient to completely melt the mixture;
b) cooling the mixture to a temperature of about 54° C., and holding at that temperature for about 4 hours;
c) cooling the mixture while mixing and optionally adding one or more additional agents.
   252. A method of preparing a foamable composition comprising a tetracycline antibiotic, at least one emollient, at least one foam adjuvant, and hydrogenated castor oil, the method comprising:
a) mixing the at least one emollient and the at least one foam adjuvant, and heating the mixture to a temperature sufficient to completely melt the mixture; and
b) cooling the mixture to a temperature of about 54° C., and holding at that temperature for about 4 hours;
c) cooling the mixture while mixing and adding a tetracyline antibiotic and optionally adding one or more additional agents.
   253. A method of preparing a foamable composition comprising a tetracycline antibiotic, a retinoid, at least one emollient, at least one foam adjuvant, and hydrogenated castor oil, the method comprising;
a) mixing the at least one emollient and the at least one foam adjuvant, and heating the mixture to a temperature sufficient to completely melt the mixture; and
b) cooling the mixture to a temperature of about 54° C., and holding at that temperature for about 4 hours;
c) cooling the mixture while mixing and adding a tetracycline antibiotic and a retinoid and optionally adding one or more additional agents.
   254. The method of any one of embodiments 249-253, wherein cooling the mixture of step (c) comprises one or more of:
a) cooling to a temperature of about 35° C. to about 40° C.;
b) cooling to a temperature of about 24° C. to about 28° C.; and/or
c) cooling to a temperature of about 22° C. to about 28° C.;
   255. The composition of any of embodiments 249-254, wherein a tetracycline antibiotic is added during step (c).
   256. The composition of any of embodiments 249-255, wherein a retinoid is added during step (c).
   257. The composition of any of embodiments 249-256, further comprising after step (c), stirring the mixture for up to 24 hours at a temperature of about 20° C. to about 26° C..
   258. The method of any one of embodiments 249-257, wherein the at least one foam adjuvant comprises a mixture of wax and non-wax foam adjuvants.
   259. The method of embodiment 258, wherein the at least one foam adjuvant is stearic acid, docosaol, stearyl alcohol, cetostearyl alcohol, myristyl alcohol, white wax, emulsified wax, or paraffin.
   260. The method of any one of embodiments embodiment 249-259, wherein the at least one emollient is coconut oil, light mineral oil, isopropyl myristate, soybean oil, or cyclomethicone.
   261. The method of any one of embodiments 249-260, wherein the tetracycline antibiotic is tetracycline, oxytetracycline, demeclocycline, doxycycline hyclate, lymecycline, meclocycline, methacycline, minocycline hydrochloride, rolitetracycline, chlorotetracycline, or tigecycline.
   262. The method of any one of embodiments 249-261, wherein the tetracycline antibiotic is minocycline hydrochloride.
   263. The method of any one of embodiments 249-262, wherein the emollient is coconut oil, light mineral oil, isopropyl myristate, or soybean oil.
   264. The method of any one of embodiments 249-263, further comprising a step of pressurizing the mixture with a propellant, e.g., Propellant AP-70, in an aerosol container.
   265. The method of any one of embodiments 249-264, wherein the foamable composition forms a foam that comprises by area more crystals with nonuniform shapes than crystals with regular shapes, e.g., spherulites, or lacks spherulites as measured by transmission electron microscopy.
   266. The method of any one of embodiments 249-265, wherein the foamable composition is shakable for at least 1 day at 5° C.
   267. The method of any one of embodiments 249-265, wherein the foamable composition is shakable for at least 14 days at 5° C.
   268. The method of any one of embodiments 249-265, wherein the foamable composition is shakable for at least 15 days at 5° C.
   269. The method of any one of embodiments 249-265, wherein the foamable composition is shakable for at least 30 days at 5° C.
   270. The method of any one of embodiments 249-265, wherein the foamable composition is shakable for at least 60 days at 5° C.
   271. The method of any one of embodiments 249-265, wherein the foamable composition is shakable for at least 90 days at 5° C.
   272. The method of any one of embodiments 249-265, wherein the foamable composition is shakable for at least 180 days at 5° C.
   273. The method of any one of embodiments 249-265, wherein the foamable composition is shakable for at least 1 day at 25° C.
   274. The method of any one of embodiments 249-265, wherein the foamable composition is shakable for at least 14 days at 25° C.
   275. The method of any one of embodiments 249-265, wherein the foamable composition is shakable for at least 15 days at 25° C.
   276. The method of any one of embodiments 249-265, wherein the foamable composition is shakable for at least 30 days at 25° C.
   277. The method of any one of embodiments 249-265, wherein the foamable composition is shakable for at least 60 days at 25° C.
   278. The method of any one of embodiments 249-265, wherein the foamable composition is shakable for at least 90 days at 25° C.
   279. The method of any one of embodiments 249-265, wherein the foamable composition is shakable for at least 180 days at 25° C., e.g. for at least 10 months, e.g., for at least 12 months, or e.g., for at least 24 months.
   280. The method of any one of embodiments 249-265, wherein the foamable composition is shakable for at least 1 day at 40° C.
   281. The method of any one of embodiments 249-265, wherein the foamable composition is shakable for at least 14 days at 40° C.
   282. The method of any one of embodiments 249-265, wherein the foamable composition is shakable for at least 15 days at 40° C.
   283. The method of any one of embodiments 249-265, wherein the foamable composition is shakable for at least 30 days at 40° C.
   284. The method of any one of embodiments 249-265, wherein the foamable composition is shakable for at least 60 days at 40° C.
   285. The method of any one of embodiments 249-265, wherein the foamable composition is shakable for at least 90 days at 40° C.
   286. The method of any one of embodiments 249-265, wherein the foamable composition is shakable for at least 180 days at 40° C.
   287. A foam produced from a foamable composition prepared by the method of any one of embodiments 249-286, wherein the foam comprises by area more crystals with Tmh crystals than crystals with regular shapes, e.g., spherulites, as measured by transmission electron microscopy.
   288. The foamable composition of embodiment 287, wherein the foam comprises by area more crystals with Tmh crystals than crystals with regular shapes, e.g., spherulites, as compared to a comparable foamable composition produced by a process that does not comprise a step of cooling the mixture to a temperature of about 54° C., and holding at that temperature for about 4 hours as measured by transmission electron microscopy.
   289. A foam produced from a foamable composition prepared by the method of any one of embodiments 249-286, wherein a sample of the foam comprises more Tmh crystals (e.g., total count or average density) than crystals with regular shapes, e.g., spherulites.
   290. The foamble composition of embodiment 289, wherein the foam comprises more Tmh crystals (e.g., total count or average density) than crystals with regular shapes, e.g., spherulites, as compared to a comparable foamable composition produced by a process that does not comprise a hold step, e.g., a step of cooling the mixture to a temperature of about 54° C., and holding at that temperature for about 4 hours.
   291. The foamable composition of embodiment 289 or 290, wherein the composition has a melting temperature about 70° C. or above.
   292. The foamable composition of any one of embodiments 289-291, wherein the composition has a melting temperature higher than a comparable foamable composition produced by a process that does not comprise a step of cooling the mixture to a temperature of about 54° C., and holding at that temperature for about 4 hours.
   293. The foamable composition of any one of embodiments 289-292, wherein the tetracycline antibiotic is minocycline and wherein the retinoid is adapalene.
   294. The foamable composition of embodiment 293, wherein minocycline is present at about 3% by weight of the composition.
   295. The foamable composition of embodiment 293 or 294, wherein adapalene is present at about 0.3% by weight of the composition.
   296. A foam produced from the foamable composition of any one of embodiments 292-295.
   297. A foamable composition comprising: a tetracycline antibiotic and/or a retinoid, and a hydrogenated castor oil, wherein the composition comprises crystals having a fingerprint providing a foam having a G′ value between about 50 Pa and about 100 Pa at 25° C.
   298. A composition comprising:
a) a wax, a foam adjuvant, or mixtures thereof;
b) a wax comprising a hydrogenated castor oil, a foam adjuvant, or mixtures thereof;
c) a wax, a foam adjuvant, or mixtures thereof and one or more therapeutic agents; or
d) a wax comprising a hydrogenated castor oil, a foam adjuvant, or mixtures thereof and one or more therapeutic agents;
   wherein the composition comprises crystals having a fingerprint comprising one or more of:
a. Tmh crystals having a cross-sectional area of about 40-150 μm² on average (e.g., about 50-130 μm² on average, e.g., about 55-70 μm² on average, e.g., about 61-63 μm² on average, e.g. about 100-122 μm² on average);
b. a larger percentage by area of Tmh crystals as compared to the percentage of spherulite or plate crystals in the composition (e.g., at least about 25% Tmh crystals, e.g., at least about 30%, e.g., at least about 33%, e.g., about 30-33% Tmh crystals and less than about 20-23% spherulites and about 6-10% plates);
c. a larger percentage of Tmh crystals in a sample of the composition as compared to the percentage of spherulites in the composition (e.g., at least about 60% Tmh crystals, e.g., at least about 80%, e.g. at least about 90%, e.g. about 100% Tmh crystals);
d. a melting temperature above 50° C. as measured by differential scanning calorimetry (DSC), e.g., a melting temperature above 60° C., e.g., a melting temperature of about 50-80° C.;
e. a melting temperature higher than observed for a composition lacking Tmh crystals or a composition having a smaller percentage by area of Tmh crystals as compared to the percentage of spherulite or plate crystals in the composition;
f. a higher enthalpy (e.g., at least about 10% more, at least about 35% more, or at least about 100% more) than observed for a composition lacking Tmh crystals or a composition having a smaller percentage by area of Tmh crystals as compared to the percentage of spherulite or plate crystals in the composition;
g. a stronger hydrogen bond between crystals (e.g., a shift to a lower frequency and/or higher intensity waveband, as measured by FTIR) than observed for a composition lacking Tmh crystals or having a smaller percentage by area of Tmh crystals as compared to the percentage of spherulite or plate crystals in the composition;
h. Tmh crystals having stronger interactions between unit cells, wherein the unit cells are tangled fibers;
i. a crystal pattern substantially similar to a crystal pattern for a holding process shown in any subpart of FIG. 2; and/or
j. an average intensity by small angle X-ray scattering higher (e.g. at least 50% more, at least about 100% or at least about 200%) than that observed for a composition lacking Tmh crystals or a composition having a smaller percentage by area of Tmh crystals as compared to the percentage of spherulite or plate crystals in the composition.
   299. The composition of embodiment 298, wherein the composition is a foamable composition.
   300. The composition of embodiment 298 or 299, wherein the composition comprises about 1.2% hydrogenated castor oil.
   301. The composition of any one of embodiments 298-300, wherein the composition is prepared by a process comprising:
a) formulating a mixture comprising the wax, the foam adjuvant, and if present, the hydrogenated castor oil;
b) heating the mixture to a temperature sufficient to completely melt the mixture;
c) cooling the mixture to a temperature of about 50-60° C., e.g., about 54° C., and maintaining the mixture at that temperature for about 1-2 or 2-16 hours, e.g., about 2-6 hours, e.g., about 4 hours;
d) cooling the mixture to a temperature of about 35° C. to about 40° C.;
e) adding an active agent, if present;
f) cooling the mixture to a temperature of about 24° C. to about 28° C.;
g) optionally adding a second or further active agent to the mixture;
h) cooling the mixture to a temperature of about 22° C. to about 28° C.; and
i) stirring the mixture for up to 24 hours at a temperature of about 20° C. to about 26° C..
   302. A composition prepared by a process comprising:
a) formulating a mixture comprising at least one emollient, at least one foam adjuvant, and hydrogenated castor oil;
b) heating the mixture to a temperature sufficient to completely melt the at least one emollient, the at least one foam adjuvant, and the hydrogenated castor oil;
c) cooling the mixture to a temperature of about 50-60° C., e.g., about 54° C., and maintaining the mixture at that temperature for about 1-2 or 2-16 hours, e.g., about 2-6 hours, e.g., about 4 hours;
d) cooling the mixture to a temperature of about 35° C. to about 40° C.;
e) adding a tetracycline antibiotic, and cyclomethicone;
f) cooling the mixture to a temperature of about 24° C. to about 28° C.;
g) adding a retinoid to the mixture;
h) cooling the mixture to a temperature of about 22° C. to about 28⁹C; and
i) stirring the mixture for up to 24 hours at a temperature of about 20° C. to about 24° C.
   303. The composition of embodiment 302, wherein the composition comprises:
i) about 50% by weight of soybean oil;
ii) about 23.6% by weight of coconut oil;
iii) about 3.3% to about 6.6% by weight of light mineral oil;
iv) about 3% by weight of stearic acid;
v) about 1.1% by weight of docosanol;
vi) about 1.2% by weight of hydrogenated castor oil;
vii) about 3.5% by weight of cetostearyl alcohol;
viii) about 2% by weight of white wax (such as beeswax);
ix) about 1.5% by weight of stearyl alcohol;
x) about 2.5% by weight of myristyl alcohol;
xi) about 5% by weight of cyclomethicone;
xii) about 3% by weight of a minocycline; and
xiii) about 0.3% by weight of adapalene.
   304. The composition of embodiment 303, wherein the minocycline is minocycline hydrochloride.
   305. The composition of embodiment 302, wherein the composition has a phase transition temperature TM4 of about 50-80° C., e.g., about 68-69° C. or about 68-72° C., as measured by DSC.
   306. A composition comprising:
i) about 45-55% (e.g., about 50%) by weight of soybean oil;
ii) about 20-30% (e.g., about 23.6%) by weight of coconut oil;
iii) about 2-10/6 (e.g., about 3.3% to about 6.6%) by weight of light mineral oil;
iv) about 1-5% (e.g., about 3%) by weight of stearic acid;
v) about 1-3% (e.g., about 1.1%) by weight of docosanol;
vi) about 1-3% (e.g., about 1.2%) by weight of hydrogenated castor oil;
vii) about 1-5% (e.g., about 3.5%) by weight of cetostearyl alcohol;
viii) about 1-5% (e.g., about 2%) by weight of white wax (such as beeswax);
ix) about 1-5% (e.g., about 1.5%) by weight of stearyl alcohol;
x) about 1-5% (e.g., about 2.5%) by weight of myristyl alcohol;
xi) about 1-10% (e.g., about 5%) by weight of cyclomethicone;
wherein the composition has a phase transition temperature T_{M4} of about 50-80° C., e.g., 68-69° C. or about 68-72° C., as measured by DSC; or a wavenumber band of about 3301-3312 cm⁻¹ when stored at 25° C., or a wavenumber band of about 3320-3324 cm⁻¹ when stored at 50° C. as measured by FTIR.
   307. The composition of any one of embodiments 299-302, wherein the composition further comprises about 3% by weight of a minocycline; and about 0.3% by weight of adapalene.
   308. The composition of embodiment 307, wherein the minocycline is minocycline hydrochloride.
   309. The composition of embodiment 307 or 308, wherein the composition comprises crystals having a fingerprint comprising one or more of:
a) Tmh crystals having a cross-sectional area of about 40-150 μm² on average (e.g., about 50-130 μm² on average, e.g., about 55-70 μm² on average, e.g., about 61-63 μm² on average, e.g. about 100-122 μm² on average);
b) a larger percentage by area of Tmh crystals as compared to the percentage of spherulite or plate crystals in the composition (e.g., at least about 25% Tmh crystals, e.g., at least about 30%, e.g., at least about 33%, e.g., about 30-33% Tmh crystals and less than about 20-23% spherulites and about 6-10% plates);
c) a larger percentage of Tmh crystals in a sample as compared to the percentage of spherulites in the composition (e.g., at least about 60% Tmh crystals, e.g., at least about 80%, e.g. at least about 90%, e.g. about 100% Tmh crystals);
d) a melting temperature above 50° C. as measured by differential scanning calorimetry (DSC), e.g., a melting temperature above 60° C., e.g., a melting temperature of about 50-80° C.;
e) a melting temperature higher than observed for a composition lacking Tmh crystals or having a smaller percentage by area of Tmh crystals as compared to the percentage of spherulite or plate crystals in the composition;
f) a higher enthalpy (e.g., at least about 10% more, at least about 35% more, or at least about 100% more, or about 110% or more) than observed for a composition lacking Tmh crystals or having a smaller percentage by area of Tmh crystals as compared to the percentage of spherulite or plate crystals in the composition;
g) a stronger hydrogen bond between crystals (e.g., a shift to a lower frequency and/or higher intensity waveband, as measured by FTIR) than observed for a composition lacking Tmh crystals or having a smaller percentage by area of Tmh crystals as compared to the percentage of spherulite or plate crystals in the composition;
h) Tmh crystals having stronger interactions between unit cells, wherein the unit cells are tangled fibers;
i) a crystal pattern substantially similar to a crystal pattern for a holding process shown in any subpart of FIG. 2; and/or
j) an average intensity by small angle X-ray scattering higher (e.g. at least 50% more, at least about 100% or at least about 200%) than that observed for a composition lacking Tmh crystals or having a smaller percentage by area of Tmh crystals as compared to the percentage of spherulite or plate crystals in the composition.
   310. The composition of any one of embodiments 301-303, wherein the composition is prepared by a method comprising heating and mixing some or all components of the composition, cooling the mixture to a temperature of about 50-60° C., e.g., about 54° C., and maintaining the mixture at that temperature for about 2-16 hours, e.g., about 2-6 hours, e.g., about 4 hours.
   311. The composition of any preceding embodiment wherein the crystals have a Tm4 above 50° C. as measured by differential scanning calorimetry (DSC), e.g., a Tm4 above 60° C., or above 64° C., e.g., Tm4 of about 50-80° C. or about 64-72° C.
   312. The composition of any preceding embodiment, wherein when the composition is kept at 5° C. for 15 days it has crystals of a Tm4 above 67° C. as measured by differential scanning calorimetry (DSC), e.g., a Tm4 above 68° C., e.g., a Tm4 of about 67-80° C.
   313. The composition of any preceding embodiment wherein when the composition is kept at 5° C. for 30 days it has crystals of a Tm4 above 67° C. as measured by differential scanning calorimetry (DSC), e.g., a Tm4 above 68° C., e.g., a Tm4 of about 67-80° C.
   314. The composition of any preceding embodiment wherein when the composition is kept at 5° C. for 15 days it has crystals of a Tm4 that is higher than that of crystals in a composition lacking Tmh crystals or a composition having a smaller percentage by area of Tmh crystals as compared to the percentage of spherulite or plate crystals in the composition.
   315. The composition of any preceding embodiment wherein when the composition is kept at 5° C. for 30 days it has crystals of a Tm4 that is higher than that of crystals in a composition lacking Tmh crystals or a composition having a smaller percentage by area of Tmh crystals as compared to the percentage of spherulite or plate crystals in the composition.
   316. The composition of any preceding embodiment wherein when the composition is kept at 25° C. on day 0 it has crystals of a Tm4 above 67° C. as measured by differential scanning calorimetry (DSC), e.g., a Tm4 above 69° C., e.g., a Tm4 of about 67-80° C.
   317. The composition of any preceding embodiment wherein when kept at 25° C. for 15 days has crystals of a Tm4 above 67° C. as measured by differential scanning calorimetry (DSC), e.g., a Tm4 above 69° C., e.g., a Tm4 of about 67-80° C.
   318. The composition of any preceding embodiment wherein when kept at 25° C. for 30 days has crystals of a Tm4 above 67° C. as measured by differential scanning calorimetry (DSC), e.g., a Tm4 above 68° C., e.g., a Tm4 of about 67-80° C.
   319. The composition of any preceding embodiment wherein when kept at 25° C. on day 0 has crystals of a Tm4 that is higher than that of crystals in a composition lacking Tmh crystals or a composition having a smaller percentage by area of Tmh crystals as compared to the percentage of spherulite or plate crystals in the composition.
   320. The composition of any preceding embodiment wherein when kept at 25° C. for 15 days has crystals of a Tm4 that is higher than that of crystals in a composition lacking Tmh crystals or a composition having a smaller percentage by area of Tmh crystals as compared to the percentage of spherulite or plate crystals in the composition.
   321. The composition of any preceding embodiment wherein when kept at 25° C. for 30 days has crystals of a Tm4 that is higher than that of crystals in a composition lacking Tmh crystals or a composition having a smaller percentage by area of Tmh crystals as compared to the percentage of spherulite or plate crystals in the composition.
   322. The composition of any preceding embodiment wherein when kept at 25° C. has no TM4.1 and/or TM4.2.
   323. The composition of any preceding embodiment wherein the crystals have a higher enthalpy (e.g. 1.2 times higher, 1.5 times higher, 2 times higher, 2.5 times higher, 3 times higher, 3.5 times higher, 4 times higher, or 5 times higher, or 10 times higher) than that of crystals in a composition lacking Tmh crystals or that of a composition having a smaller percentage by area of Tmh crystals as compared to the percentage of spherulite or plate crystals in the composition.
   324. The composition of any preceding embodiment wherein the composition has a wavenumber band of about 3301-3312 cm⁻¹ when stored at 25° C. as measured by FTIR.
   325. The composition of any preceding embodiment wherein the composition has a wavenumber band of about 3320-3324 cm⁻¹ when stored at 50° C. as measured by FTIR.
   326. The composition of any preceding embodiment wherein the composition has a band of lower frequency as measured by FTIR than that of a composition lacking Tmh crystals or a composition having a smaller percentage by area of Tmh crystals as compared to the percentage of spherulite or plate crystals in the composition.
   327. The composition of any preceding embodiment wherein the composition has a band of higher intensity as measured by FTIR than that of a composition lacking Tmh crystals or a composition having a smaller percentage by area of Tmh crystals as compared to the percentage of spherulite or plate crystals in the composition.
   328. The composition of any preceding embodiment wherein the composition has higher hydrogen bonds between crystals as measured by FTIR than that of a composition lacking Tmh crystals or a composition having a smaller percentage by area of Tmh crystals as compared to the percentage of spherulite or plate crystals in the composition.
   329. The composition of any preceding embodiment wherein the composition has a crystal pattern substantially similar to a crystal pattern for a holding process shown in any subpart of FIG. 2A.
   330. The composition of any preceding embodiment wherein the composition has a crystal pattern substantially similar to a crystal pattern for a holding process shown in any subpart of FIG. 2B.
   331. The composition of any preceding embodiment wherein the composition has a crystal pattern substantially similar to a crystal pattern for a holding process shown in any subpart of FIG. 2D.
   332. The composition of any preceding embodiment wherein the composition has a crystal pattern substantially similar to a crystal pattern for a holding process shown in any subpart of FIG. 2E.
   333. The composition of any preceding embodiment wherein the composition has a crystal pattern substantially similar to a crystal pattern for a holding process shown in any subpart of FIG. 2F.
   334. The composition of any preceding embodiment wherein the composition has a crystal pattern substantially similar to a crystal pattern for a holding process shown in any subpart of FIG. 2G.
   335. The composition of any preceding embodiment wherein the composition has an average intensity of 0.033 cm⁻¹ at 29=0.85 as measured by small x-ray scattering.
   336. The composition of any preceding embodiment wherein the composition has an average intensity of 0.031 cm⁻¹ at 20=0.7-1.2 as measured by small x-ray scattering.
   337. The composition of any preceding embodiment wherein the composition has an average intensity as measured by small x-ray scattering that is at least twofold higher than a composition lacking Tmh crystals or a composition having a smaller percentage by area of Tmh crystals as compared to the percentage of spherulite or plate crystals in the composition.
   338. The composition of embodiment 1, wherein the composition comprises Tmh crystals and plates, wherein the average percentage by area of Tmh crystals exceeds the average percentage by area of plates, as measured in a sample of the composition.
   339. The composition of embodiment 1, wherein the composition comprises Tmh crystals and spherulites, wherein the average percentage by area of Tmh crystals exceeds the average percentage by area of spherulites, as measured in a sample of the composition.
   340. The composition of embodiment 1, wherein the composition comprises Tmh crystals and plates, wherein the average percentage by area of Tmh crystals is about 30%, as measured in a sample of the composition.
   341. The composition of embodiment 1, wherein the composition comprises Tmh crystals and plates, wherein the average percentage by area of plate crystals is about 6%, as measured in a sample of the composition.
   342. The composition of embodiment 1, wherein the composition comprises Tmh crystals and spherulites, wherein the average percentage by area of Tmh crystals is about 4%-17%, as measured in a sample of the composition.
   343. The composition of embodiment 1, wherein the composition comprises Tmh crystals and spherulites, wherein the average percentage by area of spherulites is about 0-0.6%, as measured in in a sample of the composition.
   344. The composition of embodiment 1, wherein the composition comprises Tmh crystals and spherulites, wherein the average percentage of Tmh crystals is about 85%-100% of the total crystals in the sample, as measured in in a sample of the composition.
   345. The composition of embodiment 1, wherein the composition comprises Tmh crystals and spherulites, wherein the average percentage of spherulites is about 0-15% of the total crystals in the sample, as measured in a sample of the composition.
   346. The composition of embodiment 1, wherein the composition comprises Tmh crystals and plates, wherein the average cross-sectional size of Tmh crystals is about 40-80 μm², as measured in a sample of the composition.
   347. The composition of embodiment 1, wherein the composition comprises Tmh crystals and plates, wherein the average cross-sectional size of plate crystals is about 10-20 μm², as measured in a sample of the composition.
   348. The composition of embodiment 1, wherein the composition comprises spherulites, wherein the average cross-sectional size of spherulites is about 20-30 μm², as measured in a sample of the composition.
   349. The composition or method of any proceeding embodiment, wherein the fingerprint is measured at room temperature (e.g., at about 22-24° C. or about 25° C.) or at or about 40° C..
   350. The composition or method of embodiment 349, wherein the fingerprint is measured at room temperature (e.g., at about 22-24° C. or about 25° C.).
   351. The composition or method of embodiment 349, wherein the fingerprint is measured at or about 40° C..

### Embodiments Relating to Shear

1. In one or more embodiments, a manufacturing process described herein comprises mixing. In some embodiments mixing is without shear. In some embodiments mixing is with shear. In some embodiments one or more heating steps are with mixing. In some embodiments one or more cooling steps are with mixing. In some embodiments the holding process is with mixing.
2. In one or more embodiments mixing is with a mixing means, such as a rotating means e.g., one or more rotating blades or propellers or paddles or spindles, as would be appreciated by one skilled in the art. In some embodiments all the steps are with mixing.
3. In some embodiments mixing may be through a pumping means, for example a mechanical or electrical pump which pumps process materials at or near the bottom of the manufacturing container and recirculates them back at or towards the top of the container. In some embodiments pumping is through a loop outside the container. In some embodiments pumping is through a loop inside the container. In some embodiments the loop is at about the same temperature of the container. In some embodiments the external loop is at a lower temperature than that in the container, for example, if the container is at about 55° C. the external loop may be at room temperature e.g. 25° C. In some embodiments the external loop is at higher temperature than that in the container, for example, if the container is at about 22° C. the loop may be at room temperature e.g. 25° C. In some embodiments the external loop may be at a controlled temperature, which can be higher, lower or about the same as that in the container. In some embodiments there is no loop. In some embodiments all the steps are with pumping.
4. In some embodiments the homogenization is used for mixing and in others it is not. In some embodiments the mixing may be by homogenization involving shear (e.g., a high shear, a medium shear or a low shear). In some embodiments the homogenization may be used to drive the circulation of process materials. In some embodiments, for example, through a loop instead of a pump. In some embodiments there is no loop and the homogenization can facilitate circulation in the container. In some embodiments the homogenization is used during or after adding an active ingredient e.g., for a short period to facilitate rapid distribution of an active so that it is homogenous in the formulation. In some embodiments there may be one, two or more periods of homogenizations. In some embodiments the homogenization is for a short period, such as about 1 to 15 minutes, e.g., about 2.5 minutes, about 5 minutes, about 7.5 minutes, about 10 minutes, about 12.5 minutes, or about 15 minutes. In some embodiments the homogenization is for a medium period such as about 16 to 60 minutes, e.g., about 20 minutes, about 30 minutes, about 40 minutes, about 50 minutes, or about 60 minutes. In some embodiments the homogenization is for a longer period, such as about more than an hour to about 16 hours e.g., about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 16 hours, or about 16 or more hours. In some embodiments the homogenizer is in a vertical position. In some embodiments the homogenizer is in a horizontal position. In some embodiments the homogenizer is at an angle, for example, about 5 degrees, about 10 degrees, about 15 degrees about 20 degrees, about 25 degrees, about 30 degrees or about 35 degrees from vertical. In some embodiments the homogenization is at a high speed. In some embodiments the homogenization is at a medium speed. In some embodiments the homogenization is at a moderate or at a lower speed. In some embodiments when homogenization is at high speed it is for a short period. In some embodiments when homogenization is at a medium speed it is for a medium period. In some embodiments when homogenization is at a medium speed it is for a short period. In some embodiments when homogenization is at a moderate or lower speed it is for a short period. In some embodiments when homogenization is at a moderate or lower speed it is for a medium period. In some embodiments when homogenization is at a moderate or lower speed it is for a longer period. In one or more embodiments the homogenizer acts as a pump and provides shear. In some embodiments one or more or all the steps are with homogenization. In some embodiments, homogenization is not used. In one or more embodiments, high shear comprises applying a rotation of about 500-2000 rpm, e.g., generated utilizing a rotor-stator having a gap of 0.5 mm, a diameter of 20 mm and a speed of 1000 rpm or 2000 rpm. In one or more embodiments, high shear is generated utilizing a rotor-stator having a gap of 4 mm, a diameter of 160 mm and a speed of 600 rpm or 1000 rpm. In one or more embodiments, high shear is generated utilizing a rotor-stator having a gap of 4 mm, a diameter of 180 mm and a speed of 600 rpm or 1000 rpm. In one or more embodiments, high shear is generated utilizing a rotor-stator having a gap of 4 mm, a diameter of 240 mm and a speed of 600 rpm or 1000 rpm.
5. In some embodiments mixing may be by a combination of rotation and pumping. In some embodiments mixing may be by a combination of rotation and homogenization. In some embodiments mixing may be by a combination of pumping and homogenization. In some embodiments mixing may be by a combination of rotation and pumping and homogenization.
6. In one or more embodiments, the manufacturing process does not include mixing the product at high shear or energy in one or more manufacturing steps. In one or more embodiments, the manufacturing process does not include mixing the product at high shear or energy during one or more holding steps. In one or more embodiments, the manufacturing process does not include mixing the product at high shear or energy after one or more holding steps. In one or more embodiments, the manufacturing process does not include mixing the product at high shear or energy during one or more cooling steps. In one or more embodiments, the manufacturing process does not include mixing the product at high shear during one or more mixing steps.
7. In one or more embodiments, the manufacturing process does not include mixing the product at medium shear or energy in one or more manufacturing steps. In one or more embodiments, the manufacturing process does not include mixing the product at medium shear or energy during one or more holding steps. In one or more embodiments, the manufacturing process does not include mixing the product at medium shear or energy after one or more holding steps. In one or more embodiments, the manufacturing process does not include mixing the product at medium shear or energy during one or more cooling steps. In one or more embodiments, the manufacturing process does not include mixing the product at medium shear during one or more mixing steps.
8. In some embodiments mixing includes a low shear. By low shear is meant a level of shear which does not prevent or substantially reduce the formation of TMH crystals during the holding process and or does not over time eliminate or break part or all of the TMH crystals or does not over time enable part or all of them to form other crystals during one or more of the manufacturing steps, such as a cooling step or a mixing step. By high shear is meant a level of shear which over time can prevent part or all of the formation of TMH crystals during the holding process and or over time can eliminate or break part or all of the TMH crystals or can enable part or all of them to form other crystals during one or more of the manufacturing steps, such as a cooling step or a mixing step. By medium shear is meant a level of shear which can have an effect in respect of TMH crystals which is similar to that for high shear (e.g., when medium shear is applied for a longer period than high shear) or to a lesser degree than that for high shear (e.g., when medium shear is applied for the same period of time).
9. In one or more embodiments, the manufacturing process does not include mixing the product at low shear or energy in one or more manufacturing steps. In one or more embodiments. the manufacturing process does not include mixing the product at low shear or energy during one or more holding steps. In one or more embodiments, the manufacturing process does not include mixing the product at low shear or energy after one or more holding steps. In one or more embodiments, the manufacturing process does not include mixing the product at low shear or energy during one or more cooling steps. In one or more embodiments, the manufacturing process does not include mixing the product at low shear during one or more mixing steps.
10. In one or more embodiments, said shear is produced by a rotor-stator. In one or more embodiments, the rotor-stator is used in one or more steps of the manufacturing process as a pump at low speed and does not provide high shear to the product. In one or more embodiments, the use of the rotor-stator at high shear is limited to the dispersion of powders or solids (e.g. APIs) in the product.
11. In one or more embodiments, the level of shear does not prevent or substantially reduce the formation of TMH crystals during the holding process.
12. In or more embodiments, shear with mixing over time prevents part or all of the formation of TMH crystals during the holding process. In or more embodiments, shear with mixing over time breaks or eliminates part or all of the TMH crystals or enables part all of them to form other crystals during one or more of the manufacturing steps, such as a cooling step or a mixing step. In one or more embodiments the high or medium shear is applied for a period (e.g., a short period) that is insufficient to prevent formation of a majority of the TMH crystals and or to break or eliminate a majority of the TMH crystals or is insufficient to enable a majority of them to form other crystals. In one or more embodiments the mixing does not involve a high or a medium shear or energy.
13. In one or more embodiments, during the manufacturing process, mixing with high shear is applied during one or more steps after the completion of the holding. In some embodiments it is applied from after completion of the holding until the end of the manufacturing process. In one or more embodiments mixing with shear post holding is not significantly detrimental to and or does not significantly prevent the formation of Tmh crystals.
14. In one or more embodiments, during the manufacturing process, mixing with high shear is applied during one or more steps after the addition of an active ingredient. In some embodiments it is applied from after the addition of an active ingredient until the end of the manufacturing process, In one or more embodiments mixing with shear post holding after addition of one or more active ingredients is not significantly detrimental to and or does not significantly prevent the formation of Tmh crystals.
15. In one or more embodiments, Tmh crystals are modified dependent on (i) the level of shear (e.g. high, medium or low) (ii) the duration of time that shear (e.g. the more time shear is applied the greater the alteration to the fingerprint or Tmh crystals) and/or (iii) the point of time in which shear is applied (e.g before, during or after holding process).

### Sebum Dissolution Embodiments

1. In one or more embodiments the oil/wax/adjuvant vehicle of any proceeding embodiment with or without one or more drugs (e.g. a tetracycline and or a retinoid) can dissolve sebum and provide oils to the skin and thereby contribute to the treatment and a reduction of non-inflammatory lesions. In one or more further embodiments of the proceeding embodiment the vehicle with or without one or more drugs contributes to the treatment and reduction of non-inflammatory and inflammatory lesions. In one or more further embodiments the crystal fingerprint of any proceeding embodiments of the vehicle with or without one or more drugs contributes to the treatment and reduction of non-inflammatory lesions and or inflammatory lesions.

### Method of Treatment or Clinical Trial Embodiments

1. In one or more embodiments there is provided by itself or in accordance with any proceeding embodiment a method for treating a skin disorder, e.g., acne, comprising administering at least once daily to a subject in need thereof a composition or a foam composition comprising a hydrogenated oil e.g., a hydrogenated castor oil, and an emollient; and optionally one or more foam adjuvants and/or a wax capable of forming at foam.
2. There is provided the method according to embodiment 1, wherein the composition comprises one or more foam adjuvants.
3. There is provided the method according to any preceding embodiment, wherein the composition comprises a wax in addition to the hydrogenated oil.
4. There is provided the method according to any preceding embodiment, wherein the composition further comprising a tetracycline antibiotic (e.g., about 1% to about 5% e.g., about 3% by weight of a minocycline (e.g., minocycline hydrochloride)), and or a retinoid (e.g., about 0.1% to about 0.5% e.g., about 0.3% by weight of adapalene).
5. There is provided the method according to any preceding embodiment, wherein when the composition is packaged in an aerosol container and pressurized with an effective amount of liquefied or compressed gas propellant (e.g., AP-70), the composition affords upon release from the container a foam that breaks upon application of a shear force.
6. There is provided the method according to any preceding embodiment, wherein the composition comprises about 1 to about 3%, about 1 to about 2%, or about 1.2%, hydrogenated castor oil.
7. There is provided the method according to any preceding embodiment, wherein. the emollient is about 60% to 95% by weight of the composition.
8. There is provided the method according to any preceding embodiment, wherein and the foam adjuvant is about 5% to about 25% by weight of the composition.
9. There is provided the method according to any preceding embodiment, wherein the emollient comprises soybean oil (e.g., about 5% to about 90%) and or coconut oil (e.g., about 5% to about 55%).
10. There is provided the method according to any preceding embodiment, wherein the composition comprises an emollient, a foam adjuvant and a wax, and wherein the emollient comprises one or more of a soybean oil, a coconut oil, a mineral oil, and a silicone, the foam adjuvant comprises a fatty alcohol and or a fatty acid.
11. There is provided the method according to any preceding embodiment, wherein the fatty acid comprises stearic acid, and wherein the fatty alcohol comprises one or more of docosanol, stearyl alcohol, cetostearyl alcohol, and myristyl alcohol.
12. There is provided the method according to any preceding embodiment, wherein the wax comprises a white wax (such as beeswax).
13. There is provided the method according to any preceding embodiment, wherein the silicone comprises a cyclomethicone.
14. There is provided the method according to any preceding embodiment, wherein the composition comprises one or more of a) about 40% to about 60% by weight of soybean oil; b) about 20% to about 25% by weight of coconut oil; c) about 2% to about 8% by weight of light mineral oil; d) about 2% to about 4% by weight of stearic acid; e) about 0.6% to about 1.6% by weight of docosanol; f) about 1% to about 2% by weight of hydrogenated castor oil; g) about 1% to about 3% by weight of white wax (such as beeswax); h) about 1% to about 2% by weight of stearyl alcohol; i) about 2.0% to about 5.0% by weight of cetostearyl alcohol; j) about 1.8% to about 3.3% by weight of myristyl alcohol; k) about 3.0% to about 7.0% by weight of cyclomethicone; I) and about 1% to about 5% by weight of a minocycline; and m) about 0.1% to about 0.5% by weight of adapalene.
15. There is provided the method according to the preceding embodiment, wherein the composition comprises a) to l).
16. There is provided the method according to any preceding embodiment, wherein the treatment with the composition results in a reduction of non-inflammatory lesion count from baseline (e.g., ranging from about 15 to about 35), after twelve weeks of treatment.
17. There is provided the method according to the preceding embodiment, wherein the reduction is superior relative to treatment with the composition free of minocycline and or adapalene after twelve weeks of treatment.
18. There is provided the method according to any preceding embodiment, wherein the treatment with the composition results in more than about 50% reduction in non-inflammatory lesions after twelve weeks of treatment.
19. There is provided the method according to any preceding embodiment, wherein the composition results in more than about 20% reduction in non-inflammatory lesions, after about twelve weeks of treatment e.g., about a 20% to about a 60% reduction in non-inflammatory lesions, after about twelve weeks.
20. There is provided the method according to any preceding embodiment, wherein the treatment with the composition results in at least about 20%, about 25%, about 30%, about 35%, about 40%, about 45%, about 50%, about 51%, about 52%, about 53%, about 54%, about 55% or about 60% reduction in non-inflammatory lesions after about twelve weeks of treatment.
21. There is provided the method according to any preceding embodiment, wherein the treatment with the composition results in a reduction which is superior relative to treatment with the composition free of minocycline and adapalene.
22. There is provided the method according to any preceding embodiment, wherein the treatment with the composition results in a reduction of non-inflammatory lesions and wherein the reduction in non-inflammatory lesion count from baseline resultant from treatment with the same or similar composition free of both minocycline and adapalene is superior relative to a reduction with the same or similar composition free of adapalene after eight weeks or less than eight weeks of treatment.
23. There is provided the method according to any preceding embodiment, wherein the treatment with the composition results in a reduction of non-inflammatory lesions and wherein reduction in non-inflammatory lesion count from baseline resultant from treatment with the composition without minocycline is superior relative to treatment with the same or similar composition free of both minocycline and adapalene, after eight weeks or less than eight weeks of treatment.
24. There is provided the method according to any preceding embodiment, wherein the treatment with the composition results in a reduction of non-inflammatory lesions and wherein the reduction in non-inflammatory lesion count from baseline resultant from treatment with the composition is statistically significant relative to the same or similar composition free of minocycline, after twelve weeks of treatment.
25. There is provided the method according to any preceding embodiment, wherein the treatment with the composition results in a reduction of non-inflammatory lesions and wherein the reduction in non-inflammatory lesion count from baseline resultant from treatment with the composition is statistically significant relative to the same or similar composition free of adapalene after twelve weeks of treatment.
26. There is provided the method according to any preceding embodiment, wherein the treatment with the composition results in a reduction of non-inflammatory lesions and wherein the reduction of the number non-inflammatory lesions resultant from the same or similar composition free of both minocycline and adapalene is at least about 40% after twelve weeks of treatment e.g., is at least about 45% after twelve weeks of treatment.
27. There is provided the method according to any preceding embodiment, wherein the treatment with the composition results in a reduction of non-inflammatory lesions and wherein the reduction in number of non-inflammatory lesions of the subject is by about 15%, about 20%, about 25%, about 30%, about 35%, about 40%, about 45%, about 46%, about 47%, about 48%, about 49% or about 50% or more than with the same or similar composition free of minocycline and adapalene after twelve weeks of treatment.
28. There is provided the method according to any preceding embodiment, wherein the composition is safe and well tolerated when administered for a period of about twelve weeks.
29. There is provided the method according to any preceding embodiment, wherein the composition is sufficient to improve the Investigator's Global Assessment (IGA) score of the subject from baseline by at least one, two, or more grades as compared to treatment with the composition free of minocycline and adapalene and/or wherein the IGA score of the subject after treatment is clear “0” or almost clear “1”.
30. There is provided the method according to any of embodiments 1 to 21, wherein the treatment with composition results in a reduction of inflammatory lesion count from baseline (e.g., ranging from about 5 to about 15), after twelve weeks of treatment.
31. There is provided the method according to embodiment 30, wherein treatment with the composition results in reduction of inflammatory lesion count from baseline ranging from about 10 to about 30 after twelve weeks of treatment.
32. There is provided the method according to embodiment 30 or 31, wherein the reduction of inflammatory lesions is at least about 55%, e.g. about at least 60% after twelve weeks of treatment with the composition.
33. There is provided the method according to any of embodiments 30 to 32, wherein treatment with the composition results in more than about 30% reduction, e.g., about 30% to about 70%, in inflammatory lesions after twelve weeks of treatment.
34. There is provided the method according to any of embodiments 30 to 33, wherein treatment with the composition results in reduction by about 20%, about 25%, about 30%, about 35%, about 40%, about 45%, about 50%, about 55%, about 60%, about 61%, about 62%, about 63%, about 64%, or about 65% in inflammatory lesions, after twelve weeks of treatment.
35. There is provided the method according to any of embodiments 30 to 34, wherein treatment with the composition results in reduction of inflammatory lesion count from baseline is superior relative to the same or similar composition free of adapalene and minocycline after twelve weeks or less than twelve weeks of treatment.
36. There is provided the method according to any of embodiments 30 to 35, wherein treatment with the composition results reduction in inflammatory lesion that is statistically significant relative to the composition free of minocycline and adapalene after twelve weeks of treatment with composition.
37. There is provided the method according to any of embodiments 30 to 36, wherein the composition has a similar efficacy in reducing inflammatory lesions than the same or similar composition free of minocycline after twelve weeks of treatment.
38. There is provided the method according to any of embodiments 30 to 37, wherein reduction in inflammatory lesion count from baseline resultant from treatment with the composition is superior to treatment with the same or similar composition free of minocycline and/or adapalene after eight weeks of treatment.
39. There is provided the method according to any of embodiments 30 to 38, wherein reduction in inflammatory lesion count from baseline resultant from treatment with the composition is superior to treatment with (i) the same or similar composition free of minocycline and adapalene or (ii) the same or similar composition free of minocycline after four weeks or less than four weeks of treatment.
40. There is provided the method according to any of embodiments 30 to 39, wherein the reduction of inflammatory lesion count with composition is statistically significant relative to the same or similar composition free of minocycline after twelve weeks of treatment.
41. There is provided the method according to any of embodiments 30 to 40, wherein the reduction in inflammatory lesion count from baseline resultant from treatment with the composition is statistically significant to treatment with the same or similar composition free of minocycline after eight weeks of treatment.
42. There is provided the method according to any of embodiments 30 to 41, wherein the reduction in inflammatory lesion count from baseline resultant from treatment with the composition is treatment is statistically significant to treatment with the same or similar composition free of minocycline after four weeks of treatment.
43. There is provided the method according to any of embodiments 30 to 42, wherein the reduction a number of inflammatory lesions of the subject is by about 20%, about 25%, about 30%, about 35%, about 40%, about 45%, about 46%, about 47%, about 48%, about 49% or about 50% or more after twelve weeks of treatment with the same or similar composition free of minocycline and adapalene.
44. There is provided the method according to any of embodiments 30 to 43, wherein the reduction of the number of inflammatory lesions is by at least about 40%, e.g., about 45%, after twelve weeks of treatment with the same or similar composition free of minocycline and adapalene.
45. There is provided the method according to any of embodiments 30 to 44, wherein following a period of treatment wherein the composition is administered at least once daily it is administered as a maintenance dose of about every other day.
46. There is provided the method according to any of embodiments 30 to 44, wherein following a period of treatment wherein the composition is administered at least once daily it is administered as a maintenance dose of about every three days.
47. There is provided the method according to any of embodiments 30 to 45, wherein following a period of treatment wherein the composition is administered at least once daily, it is administered as a maintenance dose at a frequency including alternative days, every two days, three times a week, twice a week, once a week, once in two weeks, once in three weeks, once a month, once in two months, and alternate weeks.
48. There is provided the method according to any of embodiments 30 to 45, wherein in between a period of treatment the composition is administered as a maintenance dose at a frequency including alternative days, every two days, three times a week, twice a week, once a week, once in two weeks, once in three weeks, once a month, once in two months, and alternate weeks.
49. There is provided the method according to any of embodiments 30 to 48, wherein the maintenance dose is discontinued after a period of about one to twelve months, e.g. after three to six months.
50. There is provided the method according to any of the preceding embodiments, wherein the step of administering includes releasing the foam composition from the container and applying it onto the target area by collapsing and/or spreading it on the target area resulting in the foam's absorption onto the target area.
51. There is provided the method according to any of the preceding embodiments, wherein the composition further comprises Tmh crystals.
52. The method of any proceeding embodiment, wherein the skin disorder, is one or more of acne, rosacea, impetigo, atopic dermatitis, and psoriasis.
53. The method of embodiment 52, wherein the skin disorder, is acne.
54. The method of embodiment 52, wherein the skin disorder, is rosacea.
55. The method of embodiment 52, wherein the skin disorder, is impetigo.
56. There is provided the method according to any of embodiments 51 to 55, wherein the Tmh crystals have a fingerprint comprising one or more of:
a) a cross-sectional area of about 40-150 μm2 on average (e.g., about 50-130 μm2 on average, e.g., about 55-70 μm2 on average, e.g., about 61-63 μm2 on average, e.g. about 100-122 μm2 on average);
b) a larger percentage by area of Tmh crystals as compared to the percentage of spherulite or plate crystals in the composition (e.g., at least about 25% Tmh crystals, e.g., at least about 30%, e.g., at least about 33%, e.g., about 30-33% Tmh crystals and less than about 20-23% spherulites and about 6-10% plates);
c) a larger percentage of Tmh crystals in a sample as compared to the percentage of spherulites in the composition (e.g., at least about 60% Tmh crystals, e.g., at least about 80%, e.g. at least about 90%, e.g. about 100% Tmh crystals);
d) a melting temperature above 50° C. as measured by differential scanning calorimetry (DSC), e.g., a melting temperature above 60° C., e.g., a melting temperature of about 50-80° C.;
e) Tmh crystals having stronger interactions between unit cells, wherein the unit cells are tangled fibers;
f) a crystal pattern substantially similar to a crystal pattern for a holding process shown in any subpart of FIG. 2;
g) an average intensity by small angle X-ray scattering higher (e.g. at least 50% more, at least about 100% or at least about 200%) than that observed for a composition lacking Tmh crystals or a composition having a smaller percentage by area of Tmh crystals as compared to the percentage of spherulite or plate crystals in the composition; and or
   57. h) a raman spectra with a peak in the range 1400-1500 cm−1 having one or two shoulders, wherein the peak is at about 1446 cm−1 and has a shoulder at about 1465 cm−1 and/or has a shoulder at about 1425 cm−1.
   58. There is provided the method according to any of the embodiments of 55, 56, or 57, wherein the composition with the Tmh crystals has a lower flow point temperature (e.g., about 2.5% lower, about 5% lower, about 7.5% lower or about 10% lower or about 2° C. lower, about 3° C. lower, about 4° C. lower, about 5° C. lower or about 6° C. lower) after about 15 to 30 days of storage at 4° C. at which G″ becomes higher than G′ than that observed for the same or similar composition lacking Tmh crystals.
   59. There is provided the method according to any of the embodiments of 51 to 58, wherein the fingerprint is measured at about 25° C..
   60. There is provided the method according to any of the embodiments of 51 to 59, wherein the composition comprises about 3% by weight of a minocycline (e.g., minocycline hydrochloride), and about 0.3% by weight of a retinoid (e.g., adapalene).
   61. In one or more embodiments instead of reading the method of treatment the embodiment is alternatively read as a composition for use in any of the preceding methods.
   62. In one or more embodiments instead of reading the method of treatment the embodiment is alternatively read as use a composition in any of the preceding methods.
   63. In one or more embodiments instead of reading the method of treatment the embodiment is alternatively read as use a composition in the manufacture of a medicament having an effect on a skin disorder in any of the preceding methods.
   64. The composition of any of embodiments of the preceding methods prepared by a process comprising:
a. formulating a mixture comprising at least one emollient, at least one foam adjuvant, and a hydrogenated oil, e.g., hydrogenated castor oil;
b. heating the mixture to a temperature sufficient to completely melt the mixture;
c. cooling the mixture to a temperature of about 48-60° C., e.g., about 48-56° C., e.g., about 50-58° C., e.g., about 48-54° C., e.g., 52-56° C., e.g., about 54° C., and maintaining the mixture at that temperature for about 1-2 or about 2-72 hours, e.g., about 2-16 hours, e.g., about 2-12 hours, e.g., about 2-6 hours, e.g., about 4 hours; and
d. cooling the mixture and optionally adding one or more additional agents.
   65. The composition of embodiment 64, prepared by a process comprising:
a. formulating a mixture comprising the at least one emollient, the at least one foam adjuvant, and the hydrogenated castor oil;
b. heating the mixture to a temperature sufficient to completely melt the at least one emollient, the at least one foam adjuvant, and the hydrogenated castor oil;
c. cooling the mixture to a temperature of about 50-60° C., e.g., about 54° C., and maintaining the mixture at the temperature for about 1-72 hours, e.g., about 1-12 or 2-16 hours, e.g., about 1-2 hours or about 2-6 hours, e.g., about 4 hours;
d. cooling the mixture and optionally adding one or more additional agents.
   66. The composition of embodiments 64 or 65, wherein cooling the mixture of step (d) comprises one or more of:
a. cooling to a temperature of about 35° C. to about 40° C.;
b. cooling to a temperature of about 24° C. to about 28° C.; and/or
c. cooling to a temperature of about 22° C. to about 28° C. (e.g., about 22° C. to about 26° C.)
   67. The composition of any of embodiments 64 to 66, wherein a tetracycline antibiotic is added during step (d).
   68. The composition of any embodiments 64 to 67, wherein cyclomethicone is added during step (d).
   69. The composition of any of embodiments 64 to 68, wherein a retinoid is added during step (d).
   70. The composition of any of embodiments 64 to 69, further comprising after step (d), stirring the mixture for up to 24 hours at a temperature of about 20° C. to about 24° C.
   71. The composition of any of embodiments 64 to 70, wherein the formulation has increased stability over a composition prepared without step (c) after one or more weeks at 25° C.
   72. The composition of any of embodiments 64 to 71, wherein one or more cooling steps are with mixing.
   73. The composition of any of embodiments 64 to 71, wherein one or more holding steps are with mixing.
   74. The composition of embodiment 72 or 73, wherein the mixing is either not at high shear or energy during or after the holding process.
   75. The composition of embodiment 72 or 73, wherein mixing comprises high energy mixing or shear for one or more short periods or at reduced speeds during or after the holding process such that the Tmh crystals are substantially preserved.

### General Embodiments

1. In one or more embodiments any one or more of the aforesaid embodiments or groups of embodiments may be combined.
2. In some embodiments, one or more of the shear embodiments are applied to and combined with the exemplary embodiments.
3. In some embodiments, one or more of the sebum dissolution embodiments are applied to and combined with the exemplary embodiments.
4. In some embodiments, one or more of the methods of treatment or clinical embodiments are applied to and combined with the exemplary embodiments.
5. In some embodiments, one or more of the shear embodiments are applied to and combined with the exemplary embodiments and then sebum dissolution embodiments.
6. In some embodiments, one or more of the shear embodiments are applied to and combined with the exemplary embodiments and then sebum dissolution embodiments and then method of treatment or clinical embodiments.
7. In some embodiments, one or more of the sebum dissolution embodiments are applied to and combined with the exemplary embodiments and then shear embodiments.
8. In some embodiments, one or more of the sebum dissolution embodiments are applied to and combined with the exemplary embodiments and then shear embodiments and then method of treatment embodiments.
9. In some embodiments, one or more of the shear embodiments are applied to and combined with the exemplary embodiments and then the method of treatment or clinical embodiments.
10. In some embodiments, one or more of the shear embodiments are applied to and combined with and the exemplary embodiments and then the method of treatment or clinical embodiments and then the sebum embodiments.
11. In some embodiments, one or more of the sebum embodiments are applied to and combined with the exemplary embodiments and then the method of treatment or clinical embodiments.
12. In some embodiments, one or more of the sebum embodiments are applied to and combined with the exemplary embodiments and then the method of treatment or clinical embodiments and then the shear embodiments.
13. In some embodiments the method of treatment or clinical trial embodiments are applied to and combined with the exemplary embodiments and then the shear embodiments.
14. In some embodiments the method of treatment or clinical trial embodiments are applied to and combined with the exemplary embodiments and then the shear embodiments and then the sebum embodiments.
15. In some embodiments the method of treatment or clinical trial embodiments are applied to and combined with the exemplary embodiments and then the sebum embodiments.
16. In some embodiments the method of treatment or clinical trial embodiments are applied to and combined with the exemplary embodiments and then the sebum embodiments and then the shear embodiments.
17. In one or more embodiments there is provided a composition of any of the Examples.
18. In one or more embodiments there is provided a method of any of the Examples.

### Embodiments on TMH Crystals

1. In one or more embodiments, the TMH crystals are pure crystals. In one or more embodiments, the TMH crystals are pure crystals comprising only hydrogenated castor oil. In one or more embodiments, the TMH crystals are cocrystals. In one or more embodiments, the TMH crystals are cocrystals comprising hydrogenated castor oil. In one or more embodiments, the TMH crystals are cocrystals comprising hydrogenated castor oil and one or more waxes selected from beeswax, docosanol, stearyl alcohol, stearic acid, cetostearyl alcohol, myristyl alcohol, and mixtures thereof. In one or more embodiments, the TMH crystals are cocrystals comprising hydrogenated castor oil and one or more waxes selected from a fatty acid, a fatty alcohol and mixtures thereof. In one or more embodiments, the TMH crystals comprise a mixture of different pure crystals. In one or more embodiments, the TMH crystals comprise a mixture of different pure crystals and cocrystals.

What is claimed is:

1. A composition comprising: a hydrogenated castor oil, one or more foam adjuvant and/or a wax capable of forming a foam, and an emollient, wherein the composition comprises Tmh crystals.

2. The composition of claim 1, wherein the composition comprises
a) a phase transition temperature TM4 as measured by DSC of about 68° C. to about 73° C., e.g., about 68° C., about 69° C., about 70° C., about 71° C., or about 72° C.;
b) a raman spectra in the range of about 1400-1500 cm⁻¹ with a peak at about 1446 cm⁻¹ and having one or two shoulders at about 1465 cm⁻¹ and/or at about 1425 cm⁻¹; and/or
c) a wavenumber band of about 3301-3312 cm⁻¹ when stored measured 25° C., or about 3320-3324 cm⁻¹ when measured at 50° C. as measured by FTIR.

3. The composition of claim 1 or 2, wherein the composition comprises
a) Tmh crystals having a cross-sectional area of about 40 to about 150 μm² on average (e.g., about 50 to about 130 μm² on average);
b) a larger percentage by area of Tmh crystals as compared to the percentage of spherulite or plate crystals in the composition (e.g., a majority by area of Tmh crystals); and or
c) a larger percentage by number of Tmh crystals in a sample as compared to the percentage of spherulites in the composition (e.g., at least about 60% Tmh crystals, e.g., at least about 80%, e.g. at least about 90%, e.g. about 100% Tmh crystals).

4. The composition of any of claims 1 to 3, wherein the composition comprises one or more of:
a) a stronger hydrogen bond between crystals (e.g., a shift to a lower frequency and/or higher intensity waveband, as measured by FTIR) than observed for the same or similar composition lacking Tmh crystals or having a smaller percentage by area of Tmh crystals as compared to the percentage of spherulite or plate crystals in the composition;
b) a melting temperature higher than observed for the same or similar composition lacking Tmh crystals or the same or similar composition having a smaller percentage by area of Tmh crystals as compared to the percentage of spherulite or plate crystals in the composition;
c) a higher enthalpy than observed for the same or similar composition lacking Tmh crystals or the same or similar composition having a smaller percentage by area of Tmh crystals as compared to the percentage of spherulite or plate crystals in the composition; and/or
d) a higher average intensity by small angle X-ray scattering (e.g. at least 50% more, at least about 100% or at least about 200%) than in a composition lacking Tmh crystals or a composition having a smaller percentage by area of Tmh crystals as compared to the percentage of spherulite or plate crystals in the composition.

5. The composition of any proceeding claim, wherein the composition has a lower flow point temperature after about 15 days to about 30 days of storage at 40° C. than exhibited by the same or similar composition lacking Tmh crystals.

6. The composition of any preceding claim, wherein:
the foam adjuvant is selected from a fatty alcohol, a fatty acid and/or a wax,
the wax is selected from a plant wax, a white wax, an emulsifying wax, a carnauba wax, a candelilla wax, a cerasine/ozokerite wax, a Japan wax, a castor wax, a microcrystalline wax, a montan wax, a peat wax, an ouricury wax, a sugarcane wax, a retamo wax, a jojoba oil, an animal wax (e.g. lanolin, spermaceti wax, and wool fat), beeswax, a shellac wax, a hydrogenated oil (e.g., a soybean oil, or a hydrogenated cotton seed oil), a petroleum derived wax, a paraffin wax, polyethylene wax, and derivatives thereof, and
the emollient is selected from coconut oil, light mineral oil, isopropyl myristate, soybean oil, and cyclomethicone.

7. The composition of any preceding claim, further comprising at least one active agent.

8. The composition of claim 7, wherein the at least one active agent comprises one or more of a tetracycline antibiotic (e.g., about 3% by weight of a minocycline (e.g., minocycline hydrochloride)), and a retinoid (e.g., about 0.3% by weight of adapalene).

9. The composition of any preceding claim, wherein the composition comprises about 1-3%, about 1-2%, or about 1.2%, hydrogenated castor oil.

10. The composition of any preceding claim, wherein the composition comprises (a) about 60% to about 95% by weight of at least one emollient and (b) about 5% to about 25% by weight at least one foam adjuvant, or a combination thereof.

11. The composition of claim 10, wherein the emollient comprises soybean oil and/or coconut oil.

12. The composition of any preceding claim, wherein the composition further comprises a surfactant and or water (e.g., less than about 10%, or about 7.5%, or about 5%, or about 2.5%, or about 1%, or about 0.5%, or about 0.1% of each).

13. The composition of any preceding claim, wherein the composition is free of water and/or surfactant.

14. The composition of any preceding claim, wherein when packaged in an aerosol container and pressurized with an effective amount of liquefied or compressed gas propellant (e.g., AP-70), the composition affords upon release from the container a foam that breaks upon application of a shear force.

15. The composition of any preceding claim, wherein the composition has a phase transition temperature TM4 of above 65° C. as measured by DSC.

16. The composition of any preceding claim, wherein the composition has a phase transition temperature TM4 of above 67° C. as measured by DSC.

17. The composition any one of claims 1-16, having one or more of the following properties:
a) the Tmh crystals increase flowability or shakability of the composition, such that the composition is shakable at 25° C. for at least 180 days, or for at least 12 months, or for at least 18 months, or for at least 24 months;
b) the composition is capable of softening and/or reducing the melting temperature of sebum;
c) the composition has a melting temperature of about 70° C. or above;
d) the composition has no TM4.1 and/or TM4.2 when kept at 25° C.;
e) the composition has a at least about 10% more, at least about 35% more, or at least about 100% more, or about 110% or more higher enthalpy than that of a composition lacking Tmh crystals or that of a composition having a smaller percentage by area of Tmh crystals as compared to the percentage of spherulite or plate crystals in the composition;
f) the composition has an average intensity of 0.033 cm⁻¹ at 2θ=0.85 and or of 0.031 cm⁻¹ at 2θ=0.7−1.2 as measured by small x-ray scattering;
g) the composition has an average intensity as measured by small x-ray scattering that is at least twofold higher than a composition lacking Tmh crystals or a composition having a smaller percentage by area of Tmh crystals as compared to the percentage of spherulite or plate crystals in the composition;
h) the composition comprises Tmh crystals and plates, wherein the average percentage by area of Tmh crystals (e.g., about 30%) exceeds the average percentage by area of plates (e.g., about 6%) and or spherulites, as measured in a sample of the composition;
i) the composition comprises Tmh crystals and spherulites, wherein the average percentage by area of Tmh crystals is about 4%-17% and or the average percentage by area of spherulites is about 0-0.6%;
j) the composition comprises Tmh crystals and spherulites, wherein the average percentage of Tmh crystals is about 85%-100% and or the average percentage of spherulites is about 0-15% of the total crystals in the sample, as measured in a sample of the composition;
k) the composition comprises Tmh crystals and plates, wherein the average cross-sectional size of Tmh crystals is about 40-150 μm² (e.g., about 50-80 μm²), as measured in a sample of the composition;
l) the composition comprises Tmh crystals and plates, wherein the average cross-sectional size of plate crystals is about 10-20 μm², as measured in a sample of the composition; and/or
m) the composition comprises spherulites, wherein the average cross-sectional size of spherulites is about 20-30 μm², as measured in a sample of the composition.

18. The composition of any one of claims 1-17, wherein the composition comprises:
a) about 40% to about 60% by weight of a soybean oil; b) about 20% to about 25% by weight of a coconut oil; c) about 5% to about 15% by weight of a liquid oil; d) about 2% to about 4% by weight of fatty acid; e) about 5.4% to about 11.9% by weight of a fatty alcohol; f) about 1% to about 5% by weight of one or more waxes comprising hydrogenated castor oil

19. The composition of any one of claims 1-17, wherein the composition comprises:
a) about 40% to about 60% by weight of a soybean oil; b) about 20% to about 25% by weight of a coconut oil; c) about 2% to about 8% by weight of a mineral oil; d) about 2% to about 4% by weight of stearic acid; e) about 0.6% to about 1.6% by weight of docosanol; f) about 1% to about 2% by weight of a hydrogenated castor oil; g) about 1% to about 3% by weight of white wax (such as beeswax); h) about 1% to about 2% by weight of stearyl alcohol; i) about 2.0% to about 5.0% by weight of cetostearyl alcohol; j) about 1.8% to about 3.3% by weight of myristyl alcohol and k) about 3.0% to about 7.0% by weight of cyclomethicone.

20. The composition of claim 19, wherein the composition comprises about 1% to about 5% (such as 1%, 1.5%, 2%, 2.5%, 3%, 3.5%, 4%, 4.5%, or 5%) by weight of minocycline hydrochloride.

21. The composition of claim 20, wherein the composition comprises about 0.1% to about 0.5% (such as 0.1%, 0.2%, 0.3%, 0.4%, or 0.5%) by weight of adapalene.

22. The composition of any one of claims 1-21, for use in a method of treating or ameliorating a dermatological disorder.

23. The composition of any one of claims 1-21 in the manufacture of a medicament for treating or ameliorating a dermatological disorder.

24. The composition of claim 22 or 23, wherein the disorder is one or more of acne, rosacea, dermatological pain, dermatological inflammation, dermatitis, bacterial skin infections, fungal skin infections, viral skin infections, impetigo, pruritis, cellulitis, folliculitis, rashes, trauma or injury to the skin, post-operative or post-surgical skin conditions, eczemas, actinic keratosis, psoriasis, dermatitis, contact dermatitis, atopic dermatitis, and skin scarring.

25. A method of ameliorating, treating or preventing one or more symptoms associated with a dermatological disorder in a patient in need thereof, comprising administering the composition of any one of claims 1-21 to the patient.

26. The method of claim 25, wherein the disorder is one or more of acne, rosacea, dermatological pain, dermatological inflammation, dermatitis, bacterial skin infections, fungal skin infections, viral skin infections, impetigo, pruritis, cellulitis, folliculitis, rashes, trauma or injury to the skin, post-operative or post-surgical skin conditions, eczemas, actinic keratosis, psoriasis, dermatitis, contact dermatitis, atopic dermatitis, and skin scarring.

27. A method of treating acne or rosacea, comprising administering the composition of any one of claims 1-21 to a patient having acne or rosacea, optionally wherein the composition comprises a phase transition temperature TM4 of about 68-72° C., or about above 65° C. or 67° C. as measured by DSC.

28. The method of claim 27, wherein the one or more rosacea symptoms comprises tissue damage, telangiectasia, rhinophyma, and/or ocular rosacea, and/or the one or more acne symptoms include scars, wounds, post-inflammatory hyperpigmentation, and/or excoriated acne.

29. The method of claim 28, wherein the patient is diagnosed as at risk for tissue damage.

30. The method of any one of claims 25-29, wherein the composition comprises:
about 50% by weight of soybean oil;
about 23.6% by weight of coconut oil;
about 3.3% to about 6.6% by weight of light mineral oil;
about 3% by weight of stearic acid;
about 1.1% by weight of docosanol;
about 1.2% by weight of hydrogenated castor oil;
about 3.5% by weight of cetostearyl alcohol;
about 2% by weight of white wax (such as beeswax);
about 1.5% by weight of stearyl alcohol;
about 2.5% by weight of myristyl alcohol;
about 5% by weight of cyclomethicone;
about 3% by weight of a minocycline; and
about 0.3% by weight of adapalene.

31. A method of treating acne, comprising topically administering a foamable composition at least once daily for at least 12 weeks to a target area on the patient having acne, wherein said administration results in a reduction of lesion count from baseline or a reduction in the percent change of number of lesions from baseline, and wherein the foamable composition comprises
about 50% by weight of soybean oil;
about 23.6% by weight of coconut oil;
about 3.3% to about 6.6% by weight of light mineral oil;
about 3% by weight of stearic acid;
about 1.1% by weight of docosanol;
about 1.2% by weight of hydrogenated castor oil;
about 3.5% by weight of cetostearyl alcohol;
about 2% by weight of white wax (such as beeswax);
about 1.5% by weight of stearyl alcohol;
about 2.5% by weight of myristyl alcohol;
about 5% by weight of cyclomethicone;
about 3% by weight of a minocycline; and
about 0.3% by weight of adapalene.

32. The method of claim 31, wherein the composition comprises plates.

33. The method of claim 31 or 32, wherein the composition comprises broken Tmh crystals and/or broken spherulites

34. The method of claim 31 or 32, wherein the composition does not comprise a Tm4 above about 65° C. or above about 67° C..

35. The method of any of claims 31 to 34, wherein the minocycline comprises about 3% by weight of minocycline hydrochloride or minocycline hydrochloride in amount comparable to that of about 3% by weight of minocycline, and wherein the reduction is in inflammatory lesions and or non-inflammatory lesions and is superior to that in the absence of the minocycline at about 12 weeks of administration or earlier.

36. The method of any of claim 31 to 35, wherein the administered composition comprises minocycline and adapalene, and the treatment results in a reduction of non-inflammatory lesion count from baseline (e.g., ranging from about 15 to about 35), after twelve weeks of treatment.

37. The method of claim 36, wherein the reduction is superior to treatment with the composition free of minocycline and or adapalene.

38. The method of any one of claims 36-37, wherein treatment with the composition comprising minocycline and adapalene results in more than about 50% reduction in non-inflammatory lesions after twelve weeks of treatment.

39. The method of any one of claims 36-38, wherein the composition comprising minocycline and adapalene is administered in an amount sufficient to improve the Investigator's Global Assessment (IGA) score of the patient from baseline by at least one, two, or more grades as compared to treatment with the composition free of minocycline and adapalene, and/or wherein the composition comprising minocycline and adapalene is administered in an amount sufficient to obtain an IGA score after treatment is clear “0” or almost clear “1”.

40. The method of any one of claims 36-39, wherein treatment with the composition results in a reduction of an inflammatory lesion count from baseline ranging from about 10 to about 30, after twelve weeks of treatment.

41. The method of claim 40, wherein the reduction is superior to the reduction with a composition free of adapalene and minocycline.

42. The method of any one of claims 36-41, wherein reduction in inflammatory lesion count from baseline after treatment with the composition is superior to treatment with the composition free of minocycline and/or adapalene after eight weeks of treatment.

43. The method of claim 42, wherein the reduction in inflammatory lesion count from baseline after treatment with the composition is superior to treatment with (i) the composition free of minocycline and adapalene or (ii) the composition free of minocycline after four weeks or less than four weeks of treatment.

44. The method of any one of claims 42-43, wherein the reduction is statistically significant.

45. The method of any one of claims 40-44, wherein the reduction of inflammatory lesions is at least 55% after twelve weeks of treatment.

46. The method of any one of claims 36-45, wherein the step of administering includes releasing a foam composition from a container and applying the foam to a target area of skin on the patient by collapsing and/or spreading the foam on the target area.

47. The method of any one of claims 36-46, wherein a maintenance dose is applied following the at least 12 week treatment period at a frequency selected from:
alternative days, every two days, three times a week, twice a week, once a week, once in two weeks, once in three weeks, once a month, once in two months, and alternate weeks.

48. The method of claim 47, wherein a maintenance dose is discontinued after a period of three to six months.

49. A method of preparing a composition comprising at least one emollient and at least one wax, the method comprising;
a) mixing the at least one emollient and the at least one wax, and heating the mixture to a temperature sufficient to completely melt the mixture; and
b) cooling the mixture to a temperature of about 48° C. to about 66° C. e.g., about 54° C. or about 56° C., and holding at that temperature for about 4 hours;
c) cooling the mixture and optionally adding one or more additional agents.

50. The method of claim 49, wherein the wax comprises a hydrogenated oil (e.g., hydrogenated castor oil).

51. A method of improving crystal stability, comprising:
a) formulating a mixture comprising at least one emollient, at least one foam adjuvant, and a wax, e.g., a hydrogenated castor oil;
b) heating the mixture to a first temperature sufficient to completely melt the mixture;
c) cooling the mixture; and
d) interposing one or more disruptive steps during the cooling;
wherein the one or more disruptive steps comprises a holding period at one or more second temperatures that are below the first temperature; and wherein the second temperature is above about 37° C.

52. The method of claim 49 wherein the holding period is about 2-8 hours (e.g., about 3-6 hours, e.g., about 4-5 hours) or is for at least about 20 minutes to about 12 hours (e.g., about 2 hours, about 4 hours, about 6 hours, about 8 hours, about 10 hours, about 12 hours).

53. The method of claim 51 or 52, wherein the first temperature is about 10-90° C. higher than the second temperature.

54. The method of any one of claims 51 to 53, wherein the one or more second temperature is in a range from about 45-70° C., e.g. above 48° C., e.g., about 54° C..

55. The method of any of claims 51 to 54 further cooling the mixture and optionally adding one or more additional agents.

56. A method of preparing a foamable composition comprising at least one emollient, at least one foam adjuvant, and a hydrogenated castor oil, the method comprising;
a) mixing the at least one emollient, the at least one foam adjuvant, and hydrogenated castor oil, and heating the mixture to a temperature sufficient to completely melt the mixture;
b) cooling the mixture to a temperature of about 54° C., and holding at that temperature for about 4 hours;
c) cooling the mixture and optionally adding one or more additional agents.

57. The method of claim 56, wherein cooling the mixture of step (c) comprises one or more of:
i. cooling to a temperature of about 35° C. to about 40° C.;
ii. cooling to a temperature of about 24° C. to about 28° C.; and/or
iii. cooling to a temperature of about 22° C. to about 28° C. (e.g., 22° C. to about 28° C.).

58. The method of claim 56 or 57, wherein a tetracycline antibiotic is added during step (c).

59. The method of any of claims 56 to 58, wherein a retinoid is added during step (c).

60. A method of preparing a foamable composition comprising a tetracycline antibiotic, at least one emollient, at least one foam adjuvant, and a hydrogenated castor oil, the method comprising:
a) mixing the at least one emollient and the at least one foam adjuvant, and heating the mixture to a temperature sufficient to completely melt the mixture; and
b) cooling the mixture to a temperature of about 54° C., and holding at that temperature for about 4 hours;
c) cooling the mixture and adding a tetracyline antibiotic and optionally adding one or more additional agents.

61. A method of preparing a foamable composition comprising a retinoid, at least one emollient, at least one foam adjuvant, and a hydrogenated castor oil, the method comprising:
a) mixing the at least one emollient and the at least one foam adjuvant, and heating the mixture to a temperature sufficient to completely melt the mixture; and
b) cooling the mixture to a temperature of about 54° C., and holding at that temperature for about 4 hours;
c) cooling the mixture and adding a retinoid and optionally adding one or more additional agents.

62. A method of preparing a foamable composition comprising a tetracycline antibiotic, a retinoid, at least one emollient, at least one foam adjuvant, and a hydrogenated castor oil, the method comprising;
a) mixing the at least one emollient and the at least one foam adjuvant, and heating the mixture to a temperature sufficient to completely melt the mixture; and
b) cooling the mixture to a temperature of about 54° C., and holding at that temperature for about 4 hours;
c) cooling the mixture while mixing and adding a tetracycline antibiotic and a retinoid and optionally adding one or more additional agents.

63. The method of any of claims 60 to 62, wherein cooling the mixture of step (c) comprises one or more of:
i. cooling to a temperature of about 35° C. to about 40° C.;
ii. cooling to a temperature of about 24° C. to about 28° C.; and/or
iii. cooling to a temperature of about 22° C. to about 28° C. (e.g., about 22° C. to about 28° C.);

64. The method of any of claims 60 to 63, wherein the tetracycline antibiotic added during step (c) is a minocycline.

65. The method of any of claims 60 to 64, wherein the retinoid added during step (c) is adapalene.

66. The method of any of claims 60 to 65, further comprising after step (c), stirring the mixture for up to 24 hours (e.g., about 3 to about 16 hours) at a temperature of about 20° C. to about 24° C..

67. The method of any one of claims 60 to 66, comprising mixing during the one or more cooling steps.

68. The method of any one of claims 60 to 67, wherein the mixing is either not at high shear or energy during or after the holding process.

69. The method of any one of claims 60 to 67, wherein the mixing is at high energy or shear for short periods during and/or after the holding process.

70. The method of any one of claims 60 to 69, wherein the composition is packaged in an aerosol container and pressurized with a propellant, and the composition produces a foam upon release from the container.

71. The method of any one of claims 60 to 70, wherein the composition comprises:
a) about 50% by weight of soybean oil;
b) about 23.6% by weight of coconut oil;
c) about 3.3% to about 6.6% by weight of light mineral oil;
d) about 3% by weight of stearic acid;
e) about 1.1% by weight of docosanol;
f) about 1.2% by weight of hydrogenated castor oil;
g) about 3.5% by weight of cetostearyl alcohol;
h) about 2% by weight of white wax (such as beeswax);
i) about 1.5% by weight of stearyl alcohol;
j) about 2.5% by weight of myristyl alcohol;
k) about 5% by weight of cyclomethicone;
I) about 3% by weight of a minocycline; and
m) about 0.3% by weight of adapalene.

72. A foamable composition prepared by a process comprising:
a) formulating a mixture comprising at least one emollient, at least one foam adjuvant, and a hydrogenated castor oil;
b) heating the mixture to a temperature sufficient to completely melt the mixture;
c) cooling the mixture to a temperature of about 48-60° C., e.g., about 48-56° C., e.g., about 50-58° C., e.g., about 48-54° C., e.g., 52-56° C., e.g., about 54° C., and maintaining the mixture at that temperature for about 1-2 or about 2-72 hours, e.g., about 2-16 hours, e.g., about 2-12 hours, e.g., about 2-6 hours, e.g., about 4 hours; and
d) cooling the mixture and optionally adding one or more additional agents.

73. A foamable composition prepared by a process comprising:
a) formulating a mixture comprising the at least one emollient, the at least one foam adjuvant, and the hydrogenated castor oil;
b) heating the mixture to a temperature sufficient to completely melt the at least one emollient, the at least one foam adjuvant, and the hydrogenated castor oil;
c) cooling the mixture to a temperature of about 50-60° C., e.g., about 54° C., and maintaining the mixture at the temperature for about 1-72 hours, e.g., about 1-12 or 2-16 hours, e.g., about 1-2 hours or about 2-6 hours, e.g., about 4 hours;
d) cooling the mixture and optionally adding one or more additional agents.

74. The composition of claim 72 or 73, wherein cooling the mixture of step (d) comprises one or more of:
a) cooling to a temperature of about 35° C. to about 40° C.;
b) cooling to a temperature of about 24° C. to about 28° C.; and/or
c) cooling to a temperature of about 22° C. to about 28° C. (e.g., about 22° C. to about 26° C.);

75. The composition of any of claims 72 to 74, wherein a tetracycline antibiotic is added during step (d).

76. The composition of any of claims 72 to 75, wherein cyclomethicone is added during step (d).

77. The composition of any of claims 72 to 76, wherein a retinoid is added during step (d).

78. The composition of any of claims 72 to 77, further comprising after step (d), stirring the mixture for up to 24 hours at a temperature of about 20° C. to about 24 QC.

79. The composition of any of claims 72 to 78, wherein the formulation has increased stability over a composition prepared without step (c) after one or more weeks at 25° C..

80. The composition of any of claims 72 to 79, wherein the formulation has increased shakability and/or flowability over a composition prepared without step (c) after one or more weeks at 25° C..

81. The composition of any of claims 72 to 80, wherein the formulation has stronger hydrogen bonds as compared to a composition prepared without step (c), e.g., as measured by FTIR.

82. The composition of any of claims 72 to 81, wherein the composition comprises more Tmh crystals by area as compared to a comparable composition produced by a process that does not comprise a step of cooling the mixture to a temperature of about 50-60° C. and maintaining the mixture at the temperature for about 1-72, e.g., about 1-16, e.g., 1-2 or 2-16 hours.

83. The composition of any of claims 72 to 82, wherein the composition comprises more Tmh crystals by number as compared to a comparable composition produced by a process that does not comprise a step of cooling the mixture to a temperature of about 50-60° C. and maintaining the mixture at the temperature for about 1-72, e.g., about 1-16, e.g., 1-2 or 2-16 hours.

84. The composition of any one of claims 72 to 82, wherein one or more cooling steps are with mixing.

85. The composition of claim 84, wherein the mixing is either not at high shear or energy during or after the holding process.

86. The composition of claim 85, wherein high energy mixing or shear is for short periods or at reduced speeds during or after the holding process such that the Tmh crystals are substantially preserved.

87. The composition of any one of claims 72 to 86, wherein the composition is packaged in an aerosol container and pressurized with a propellant, and the composition produces a foam upon release from the container.

88. A composition prepared by a process comprising:
a) formulating a mixture comprising at least one emollient, at least one foam adjuvant, and a wax (e.g., a hydrogenated oil, e.g., a hydrogenated castor oil);
b) heating the mixture to a temperature sufficient to completely melt the mixture;
c) cooling the mixture to a temperature of about 50-60° C., e.g., about 54° C., and maintaining the mixture at that temperature for about 1-2 or 2-16 hours, e.g., about 2-6 hours, e.g., about 4 hours;
d) cooling the mixture to a temperature of about 35° C. to about 40° C.;
e) adding a tetracycline antibiotic, and cyclomethicone;
f) cooling the mixture to a temperature of about 24° C. to about 28° C.;
g) adding a retinoid to the mixture;
h) cooling the mixture to a temperature of about 20° C. to about 26° C.; and
i) stirring the mixture for up to 24 hours at a temperature of about 22° C. to about 24° C..

89. The composition of claim 88, wherein the composition comprises (a) about 60% to about 95% by weight of at least one emollient and (b) about 5% to about 25% by weight at least one foam adjuvant.

90. The composition of claims 88 or 89, wherein the composition comprises: a) about 40% to about 60% by weight of a soybean oil; b) about 20% to about 25% by weight of a coconut oil; c) about 2% to about 8% by weight of a mineral oil; d) about 2% to about 4% by weight of stearic acid; e) about 0.6% to about 1.6% by weight of docosanol; f) about 1% to about 2% by weight of a hydrogenated castor oil; g) about 1% to about 3% by weight of white wax (such as beeswax); h) about 1% to about 2% by weight of stearyl alcohol; i) about 2.0% to about 5.0% by weight of cetostearyl alcohol; j) about 1.8% to about 3.3% by weight of myristyl alcohol; k) about 3.0% to about 7.0% by weight of cyclomethicone; l) about 1% to about 5% (such as 1%, 1.5%, 2%, 2.5%, 3%, 3.5%, 4%, 4.5%, or 5%) by weight of minocycline hydrochloride and m) about 0.1% to about 0.5% (such as 0.1%, 0.2%, 0.3%, 0.4%, or 0.5%) by weight of adapalene.

91. The composition of any of claims 88 to 90, wherein the composition comprises:
a) about 50% by weight of soybean oil;
b) about 23.6% by weight of coconut oil;
c) about 3.3% to about 6.6% by weight of light mineral oil;
d) about 3% by weight of stearic acid;
e) about 1.1% by weight of docosanol;
f) about 1.2% by weight of hydrogenated castor oil;
g) about 3.5% by weight of cetostearyl alcohol;
h) about 2% by weight of white wax (such as beeswax);
i) about 1.5% by weight of stearyl alcohol;
j) about 2.5% by weight of myristyl alcohol;
k) about 5% by weight of cyclomethicone;
I) about 3% by weight of a minocycline; and
m) about 0.3% by weight of adapalene.

92. The composition of claim 91, wherein the minocycline is minocycline hydrochloride.

93. The composition of claims 88 to 92, wherein the one or more cooling steps are with mixing.

94. The composition of claim 93, wherein the mixing is either not at high shear or energy during or after the holding process.

95. The composition of claim 94, wherein high energy mixing or shear is for short periods or at reduced speeds during or after the holding process such that the Tmh crystals are substantially preserved

96. The composition of any of claims 88 to 95, wherein the composition has a phase transition temperature TM4 of about 64-67° C. or about 68-73° C., or about above 65° C. or about above 67° C. as measured by DSC.

97. The composition of any of claims 88 to 96, wherein the composition has a raman spectra in the range of about 1400-1500 cm⁻¹, with a peak at about 1446 cm⁻¹ and having one or two shoulders at about 1465 cm⁻¹ and/or at about 1425 cm⁻¹.

98. The composition of any of claims 88 to 97, wherein the composition has a wavenumber band of about 3301-3312 cm⁻¹ when measured at 25° C., or about 3320-3324 cm⁻¹ when measured at 50° C. as measured by FTIR.

99. The composition of any of claims 88 to 98, wherein the composition has a melting temperature higher than a comparable foamable composition produced by a process that does not comprise a step of cooling the mixture to a temperature of about 54° C., and holding at that temperature for about 4 hours.

100. The composition of any of claims 88 to 99, wherein the composition is packaged in an aerosol container and pressurized with a propellant, and the composition produces a foam upon release from the container.

101. A foam produced from a foamable composition of claim 100, wherein the foam comprises by area more Tmh crystals than crystals with regular shapes, e.g., spherulites, as measured by transmission electron microscopy.

102. The foam of claim 101, wherein the foamable composition comprises by area more Tmh crystals than spherulites, as compared to a comparable foamable composition produced by a process that does not comprise a step of cooling the mixture to a temperature of about 54° C., and holding at that temperature for about 4 hours as measured by transmission electron microscopy.

103. The foam of claim 101 or 102, wherein a sample of the foam comprises more Tmh crystals (e.g., total count or average density) than spherulites.

104. The foam of any of claims 101 to 103, wherein the foamable composition comprises more Tmh crystals (e.g., total count or average density) than spherulites, as compared to a comparable foamable composition produced by a process that does not comprise a hold step, e.g., a step of cooling the mixture to a temperature of about 54° C., and holding at that temperature for about 4 hours.

105. The composition of claim 93, wherein the manufacturing process is with mixing with high shear during holding.

106. The composition of claim 105, wherein the manufacturing process is with mixing with high shear (e.g., 500-2000 rpm) after holding.

107. The composition of claim 105, wherein the manufacturing process is with mixing with high shear during and after holding.

108. The composition of claim 105, wherein the manufacturing process is with mixing with high shear before, during, and after holding.

109. The composition of any of claims 105 to 108, wherein the composition comprises plates.

110. The composition of claim 109, wherein the composition comprises broken Tmh crystals and broken spherulites.

111. The composition of any of claims 105 to 110, wherein the composition does not comprise a Tm4 above about 65° C. or above about 67° C..

112. A method of treating acne or rosacea, comprising administering the composition of any one of claims 72-100 and 105-111 or the foam of any of claims 101-104 to a patient in need thereof.
